(19) United States
(12) Patent Application Publication
(10) Pub. No.: US 2012/0128713 A1
(43) Pub. Date: May 24, 2012

(54) Replication-Defective Flavivirus Vaccine Vectors Against Respiratory Syncytial Virus

(75) Inventors: Konstantin V. Pugachev, Natick, MA (US); Alexander A. Rumyantsev, Somerville, MA (US); Maryann Giel-Moloney, Brighton, MA (US); Mark Parrington, Bradford (CA); Linong Zhang, Maple (CA)

(73) Assignees: Sanofi Pasteur Limited, Toronto, ON (CA); Sanofi Pasteur Biologics Co., Cambridge, MA (US)

(21) Appl. No.: 13/256,565

(22) PCT Filed: Mar. 16, 2010
(86) PCT No.: PCT/US10/27552

§ 371 Date: Jan. 30, 2012

Related U.S. Application Data
(60) Provisional application No. 61/210,305, filed on Mar. 16, 2009.

(51) Int. Cl. C12N 7/00; C12N 15/45; A61K 39/295

(52) U.S. Cl. 424/199.1; 435/235.1; 536/23.72

(57) ABSTRACT

Replication-defective vaccine vectors against respiratory syncytial virus (RSV) are disclosed. Corresponding compositions and methods employing the vaccine vectors are also disclosed.

### CROSS-REFERENCE TO RELATED APPLICATIONS

This application claims benefit of U.S. provisional application No. 61/210,305, filed Mar. 16, 2009, the contents of which are incorporated herein by reference.

### FIELD OF THE INVENTION

This invention relates to replication-defective flavivirus vaccine vectors against respiratory syncytial virus (RSV), and corresponding compositions and methods.

### BACKGROUND OF THE INVENTION

Flaviviruses are distributed worldwide and represent a global public health problem. Flaviviruses also have a significant impact as veterinary pathogens. Flavivirus pathogens include yellow fever (YF), dengue types 1-4 (DEN1-4), Japanese encephalitis (JE), West Nile (WN), tick-borne encephalitis (TBE), and other viruses from the TBE serocomplex, such as Kyasanur Forest disease (KFD) and Omsk hemorrhagic fever (OHF) viruses. Vaccines against YF [live attenuated vaccine (LAV) strain 17D], JE [inactivated vaccines (INV) and LAV], and TBE (INV) are available. No licensed human vaccines are currently available against DEN and WN. Veterinary vaccines have been in use including, for example, vaccines against WN in horses (INV, recombinant and live chimeric vaccines), JE (INV and LAV) to prevent encephalitis in horses and stillbirth in pigs in Asia, louping ill flavivirus (NV) to prevent neurologic disease in sheep in the UK, and TBE (NV) used in farm animals in Czech Republic (NV) (Monath and Heinz, Flaviviruses, in Fields et al. Eds., Fields Virology, 3rd Edition, Philadelphia, New York, Lippincott-Raven Publishers, 1996, pp. 961-1034).

Flaviviruses are small, enveloped, plus-strand RNA viruses transmitted primarily by arthropod vectors (mosquitoes or ticks) to natural hosts, which are primarily vertebrate animals, such as various mammals, including humans, and birds. The flavivirus genomic RNA molecule is about 11,000 nucleotides (nt) in length and encompasses a long open reading frame (ORF) flanked by 5′ and 3′ untranslated terminal regions (UTRs) of about 120 and 500 nucleotides in length, respectively. The ORF encodes a polyprotein precursor that is cleaved co- and post-translationally to generate individual viral proteins. The proteins are encoded in the order: C-prM/M-E-NS1-NS2A/2B-NS3-NS4A/4B-NS5, where C (core/capsid), prM/M (pre-membrane/membrane), and E (envelope) are the structural proteins, i.e., the components of viral particles, and the NS proteins are non-structural proteins, which are involved in intracellular virus replication. Flavivirus replication occurs in the cytoplasm. Upon infection of cells and translation of genomic RNA, processing of the polyprotein starts with translocation of the prM portion of the polyprotein into the lumen of endoplasmic reticulum (ER) of infected cells, followed by translocation of E and NS1 portions, as directed by the hydrophobic signals for the prM, E, and NS1 proteins Amino-termini of prM, E, and NS1 proteins are generated by cleavage with cellular signalase, which is located on the luminal side of the ER membrane, and the resulting individual proteins remain carboxy-terminally anchored in the membrane. Most of the remaining cleavages, in the nonstructural region, are carried out by the viral NS2B/NS3 serine protease. The viral protease is also responsible for generating the C-terminus of the mature C protein found in progeny virions. Newly synthesized genomic RNA molecules and the C protein form a dense spherical nucleocapsid, which becomes surrounded by cellular membrane in which the E and prM proteins are embedded. The mature M protein is produced by cleavage of prM shortly prior to virus release by cellular furin or a similar protease. E, the major protein of the envelope, is the principal target for neutralizing antibodies, the main correlate of immunity against flavivirus infection. Virus-specific cytotoxic T-lymphocyte (CTL) response is the other key attribute of immunity. Multiple CD8+ and CD4+ CTL epitopes have been characterized in various flavivirus structural and non-structural proteins. In addition, innate immune responses contribute to both virus clearance and regulating the development of adaptive immune responses and immunologic memory.

In addition to the inactivated (INV) and live-attenuated (LAV) vaccines against flaviviruses discussed above, other vaccine platforms have been developed. One example is based on chimeric flaviviruses that include yellow fever virus capsid and non-structural sequences and prM-E proteins from other flaviviruses, to which immunity is sought. This technology has been used to develop vaccine candidates against dengue (DEN), Japanese encephalitis (JE), West Nile (WN), and St. Louis encephalitis (SLE) viruses (see, e.g., U.S. Pat. Nos. 6,962,708 and 6,696,281). Yellow fever virus-based chimeric flaviviruses have yielded highly promising results in clinical trials.

Another flavivirus vaccine platform is based on the use of pseudoinfectious virus (PIV) technology (Mason et al., Virology 351:432-443, 2006; Shustov et al., J. Virol. 21:11737-11748, 2007; Widman et al., Adv. Virus. Res. 72:77-126, 2008; Suzuki et al., J. Virol. 82:6942-6951, 2008; Suzuki et al., J. Virol. 83:1870-1880, 2009; Ishikawa et al., Vaccine 26:2772-2781, 2008; Widman et al., Vaccine 26:2762-2771, 2008). PIVs are replication-defective viruses attenuated by a deletion(s). Unlike live flavivirus vaccines, they undergo a single round replication in vivo (or optionally limited rounds, for two-component constructs; see below), which may provide benefits with respect to safety. PIVs also do not induce viremia and systemic infection. Further, unlike inactivated vaccines, PIVs mimic whole virus infection, which can result in increased efficacy due to the induction of robust B- and T-cell responses, higher durability of immunity, and decreased dose requirements. Similar to whole viruses, PIV vaccines target antigen-presenting cells, such as dendritic cells, stimulate toll-like receptors (TLRs), and induce balanced Th1/Th2 immunity. In addition, PIV constructs have been shown to grow to high titers in substrate cells, with little or no cytopathic effect (CPE), allowing for high-yield manufacture, optionally employing multiple harvests and/or expansion of infected substrate cells.

The principles of the PIV technology are illustrated in FIGS. 1 and 2. There are two variations of the technology. In the first variation, a single-component pseudoinfectious virus (s-PIV) is constructed with a large deletion in the capsid protein (C), rendering mutant virus unable to form infectious viral particles in normal cells (FIG. 1). The deletion does not remove the first −20 codons of the C protein, which contain an RNA cyclization sequence, and a similar number of codons at the end of C, which encode a viral protease cleavage site and the signal peptide for prM. The s-PIV can be propagated, e.g., during manufacture, in substrate (helper) cell cultures in which the C protein is supplied in trans, e.g., in stably transfected cells producing the C protein (or a larger helper cassette including C protein), or in cells containing an alphavirus replicon [e.g., a Venezuelan equine encephalitis virus (VEE) replicon] expressing the C protein or another intracellular expression vector expressing the C protein. Following inoculation in vivo, e.g., after immunization, the PIV undergoes a single round of replication in infected cells in the absence of trans-complementation of the deletion, without spread to surrounding cells. The infected cells produce empty virus-like particles (VLPs), which are the product of the prM-E genes in the PIV, resulting in the induction of neutralizing antibody response. A T-cell response should also be induced via MHCl presentation of viral epitopes. This approach has been applied to YF 17D virus and WN viruses and WN/JE and WN/DEN2 chimeric viruses (Mason et al., Virology 351:432-443, 2006; Suzuki et al., J. Virol. 83:1870-1880, 2009; Ishikawa et al., Vaccine 26:2772-2781, 2008; Widman et al., Vaccine 26:2762-2771, 2008; WO 2007/098267; WO 2008/137163).

In the second variation, a two-component PIV (d-PIV) is constructed (FIG. 2). Substrate cells are transfected with two defective viral RNAs, one with a deletion in the C gene and another lacking the prM-E envelope protein genes. The two defective genomes complement each other, resulting in accumulation of two types of PIVs in the cell culture medium (Shustov et al., J. Virol. 21:11737-11748, 2007; Suzuki et al., J. Virol. 82:6942-6951, 2008). Optionally, the two PIVs can be manufactured separately in appropriate helper cell lines and then mixed in a two-component formulation. The latter may offer an advantage of adjusting relative concentrations of the two components, increasing immunogenicity and efficacy. This type of PIV vaccine should be able to undergo a limited spread in vivo due to coinfection of some cells at the site of inoculation with both components. The spread is expected to be self-limiting as there are more cells in tissues than viral particles produced by initially coinfected cells. In addition, a relatively high MOI is necessary for efficient co-infection, and cells outside of the inoculation site are not expected to be efficiently coinfected (e.g., in draining lymph nodes). Cells infected with the AC PIV alone produce the highly immunogenic VLPs. Coinfected cells produce the two types of packaged defective viral particles, which also stimulate neutralizing antibodies. The limited infection is expected to result in a stronger neutralizing antibody response and T-cell response compared to s-PIVs. To decrease chances of recombination during manufacture or in vivo, including with circulating flaviviruses, viral sequences can be modified in both s-PIVs and d-PIVs using, e.g., synonymous codon replacements, to reduce nucleotide sequence homologies, and mutating the complementary cyclization 5′ and 3′ elements.

Respiratory syncytial virus (RSV) is a negative-sense, single-stranded RNA virus of the family Paramyxoviridae. Its name is based on the activity of the RSV fusion or F glycoprotein, which is on the surface of the virus and causes cell membranes of infected cells to merge, resulting in the formation of syncytia. RSV infects the respiratory tract, and is the major cause of lower respiratory tract infections (including pneumonia) and hospital visits during infancy and childhood. For example, in the United States, 60% of infants are infected during their first RSV season, and nearly all children will have been infected by 2-3 years of age (Glezen et al., Am. J. Dis. Child. 140(6):543-546, 1986). Of those infected, 2-3% will develop bronchiolitis, or inflammation of the small airways in the lung, and require hospitalization (Hall et al., N. Engl. J. Med. 360(6):588-598, 2009). Further, RSV infection is increasingly being found as an infection of the elderly. Current treatment is generally focused on supportive care, including administration of fluids and oxygen.

### SUMMARY OF THE INVENTION

The invention provides replication-deficient pseudoinfectious flaviviruses that each include a flavivirus genome including (i) one or more deletions or mutations in nucleotide sequences encoding one or more proteins selected from the group consisting of capsid (C), pre-membrane (prM), envelope (E), non-structural protein 1 (NS1), non-structural protein 3 (NS3), and non-structural protein 5 (NS5), and (ii) a sequence encoding a respiratory syncytial virus (RSV) peptide or protein, or a fragment or analog thereof. As described elsewhere herein, the vectors of the invention are replication deficient due to the one or more deletions or mutations, and can be complemented in trans (see below for details). Any of the deletions/mutations described herein, as well as other deletions/mutations resulting in replication deficiency, can be used in the vectors of the invention.

In one embodiment, the respiratory syncytial virus (RSV) protein is the RSV F protein, or a fragment or analog thereof. In various examples, the RSV F protein lacks a trans-membrane domain, e.g., it is truncated so that it is produced in secreted form. In other examples, the respiratory syncytial virus (RSV) protein is the RSV G protein, or a fragment or analog thereof.

In various embodiments, the one or more deletions or mutations is within capsid (C) sequences of the flavivirus genome; is within pre-membrane (prM) and/or envelope (E) sequences of the flavivirus genome; is within capsid (C), pre-membrane (prM), and envelope (E) sequences of the flavivirus genome; and/or is within non-structural protein 1 (NS1) sequences of the flavivirus genome. In other examples, the flavivirus genome includes sequences encoding a pre-membrane (prM) and/or envelope (E) protein.

The flavivirus genome of the replication-deficient pseudoinfectious flaviviruses can be, for example, selected from that of yellow fever virus, West Nile virus, tick-borne encephalitis virus, Langat virus, Japanese encephalitis virus, dengue virus (1-4), and St. Louis encephalitis virus sequences, and chimeras thereof (also see below). In certain examples, the chimeras include pre-membrane (prM) and envelope (E) sequences of a first flavivirus, and capsid (C) and non-structural sequences of a second, different flavivirus. In other examples, the genome is packaged in a particle including pre-membrane (prM) and envelope (E) sequences from a flavivirus that is the same or different from that of the genome. Further, sequences encoding the RSV protein can be inserted in the place of or in combination with the one or more deletions or mutations of the one or more proteins.

In certain examples, sequences encoding the respiratory syncytial virus peptide or protein, or a fragment or analog thereof, are inserted in the flavivirus genome within sequences encoding the envelope (E) protein, within sequences encoding the non-structural 1 (NS1) protein, within sequences encoding the pre-membrane (prM) protein, intergenically between sequences encoding the envelope (E) protein and non-structural protein 1 (NS1), intergenically between non-structural protein 2B (NS2B) and non-structural protein 3 (NS3), or as a bicistronic insertion in the 3′ untranslated region of the flavivirus genome.

The invention also includes pharmaceutical compositions including one or more of the replication-deficient pseudoinfectious flaviviruses described above and elsewhere herein. Compositions of the invention can also a pharmaceutically acceptable carrier or diluent, and, optionally, an adjuvant.

Other compositions of the invention include a first replication-deficient pseudoinfectious flavivirus, such as one of those described above and elsewhere herein, and a second, different replication-deficient pseudoinfectious flavivirus including a genome having one or more deletions or mutations in nucleotide sequences encoding one or more proteins selected from the group consisting of capsid (C), pre-membrane (prM), envelope (E), non-structural protein 1 (NS1), non-structural protein 3 (NS3), and non-structural protein 5 (NS5), wherein the one or more proteins encoded by the sequences in which the one or more deletion(s) or mutation(s) occur in the second, different replication-deficient pseudoinfectious flavivirus are different from the one or more proteins encoded by the sequences in which the one or more deletion(s) or mutation(s) occur in the first replication-deficient pseudoinfectious flavivirus.

The invention also provides methods of inducing an immune response to respiratory syncytial virus (RSV) in a subject, involving administering to the subject one or more replication-deficient pseudoinfectious flaviviruses or a composition as described above and elsewhere herein. The subject may be at risk of but not have an infection by respiratory syncytial virus (RSV), or the subject may have an infection by respiratory syncytial virus (RSV). In certain examples, the subject is an infant, young child, or elderly person. The methods of the invention can be for inducing an immune response against a protein encoded by the flavivirus genome, in addition to RSV. In such methods, the subject may be at risk of but does not have an infection by the flavivirus corresponding to the genome of the pseudoinfectious flavivirus, which includes sequences encoding a flavivirus pre-membrane and/or envelope protein. In another example, the subject has an infection by the flavivirus corresponding to the genome of the pseudoinfectious flavivirus, which includes sequences encoding a flavivirus pre-membrane and/or envelope protein.

Also included in the invention are nucleic acid molecules corresponding to the genomes of pseudoinfectious flaviviruses as described herein and complements thereof.

The invention also provides methods of making a replication-deficient pseudoinfectious flavivirus as described herein. These methods involve introducing a nucleic acid molecule as described above into a cell that expresses the protein corresponding to any sequences deleted from the flavivirus genome of the replication-deficient pseudoinfectious flavivirus. The protein can be expressed in the cell from, for example, the genome of a second, different, replication-deficient pseudoinfectious flavivirus. In various examples, the protein is expressed from a replicon (e.g., an alphavirus replicon, such as a Venezuelan Equine Encephalitis virus replicon).

By “replication-deficient pseudoinfectious flavivirus” or “PIV” is meant a flavivirus that is replication-deficient due to a deletion or mutation in the flavivirus genome. The deletion or mutation can be, for example, a deletion of a large sequence, such as most of the capsid protein, as described herein (with the cyclization sequence remaining; see below). In other examples, sequences encoding different proteins (e.g., prM, E, NS1, NS3, and/or NS5; see below) or combinations of proteins (e.g., prM-E or C-prM-E) are deleted. This type of deletion may be advantageous for use of the PIV as a vector to deliver a heterologous immunogen, as the deletion can permit insertion of sequences that may be, for example, at least up to the size of the deleted sequence. In other examples, the mutation can be, for example, a point mutation, provided that it results in replication deficiency, as discussed above. Because of the deletion or mutation, the genome does not encode all proteins necessary to produce a full flavivirus particle. The missing sequences can be provided in trans by a complementing cell line that is engineered to express the missing sequence (e.g., by use of a replicon; s-PIV; see below), or by co-expression of two replication-deficient genomes in the same cell, where the two replication-deficient genomes, when considered together, encode all proteins necessary for production (d-PIV system; see below).

Upon introduction into cells that do not express complementing proteins, the genomes replicate and, in some instances, generate “virus-like particles,” which are released from the cells and are able to leave the cells and be immunogenic, but cannot infect other cells and lead to the generation of further particles. For example, in the case of a PIV including a deletion in capsid protein encoding sequences, after infection of cells that do not express capsid, VLPs including prM-E proteins are released from the cells. Because of the lack of capsid protein, the VLPs lack capsid and a nucleic acid genome. In the case of the d-PIV approach, production of further PIVs is possible in cells that are infected with two PIVs that complement each other with respect to the production of all required proteins (see below).

The invention provides several advantages. For example, the PIV vectors and PIVs of the invention are highly attenuated and highly efficacious after one-to-two doses, providing durable immunity. Further, unlike inactivated vaccines, PIVs mimic whole virus infection, which can result in increased efficacy due to the induction of robust B- and T-cell responses, higher durability of immunity, and decreased dose requirements. In addition, similar to whole viruses, PIV vaccines target antigen-presenting cells, such as dendritic cells, stimulate toll-like receptors (TLRs), and induce balanced Th1/Th2 immunity. PIV constructs have also been shown to grow to high titers in substrate cells, with little or no CPE, allowing for high-yield manufacture, optionally employing multiple harvests and/or expansion of infected substrate cells. Further, the PIV vectors of the invention provide an option for developing vaccines against non-flavivirus pathogens, such as RSV, for which no vaccines are currently available.

Other features and advantages of the invention will be apparent from the following detailed description, the drawings, and the claims.

### BRIEF DESCRIPTION OF THE DRAWINGS

FIG. 1 is a schematic illustration of single component PIV (s-PIV) technology.

FIG. 2 is a schematic illustration of two-component PIV (d-PIV) technology.

FIG. 3 is a schematic illustration of a general experimental design for testing immunogenicity and efficacy of PIVs in mice.

FIG. 4 is a graph comparing the humoral immune response induced by PIV-WN(RV-WN) with that of YF/WN LAV (CV-WN) in mice.

FIG. 5 is a series of graphs showing the results of challenging hamsters immunized with PIV-YF (RV-YF), YF17D, PIV-WN(RV-WN), and YF/WN LAV (CVWN) with hamster-adapted Asibi (PIV-YF and YF 17D vaccinees) and wild type WN-NY99 (PIV-WN and YF/WN LAV vaccinees).

FIG. 6 is a table showing YF/TBE and YF/LGT virus titers and plaque morphology obtained with the indicated chimeric flaviviruses.

FIG. 7 is a table showing WN/TBE PIV titers and examples of immunofluorescence of cells containing the indicated PIVs.

FIG. 8 is a set of graphs showing the replication kinetics of YF/TBE LAV and PIV-WN/TBE in Vero and BHK cell lines (CV-Hypr=YF/Hypr LAV; CV-LGT=YF/LGT LAV; RV-WN/TBEV=PIV-WN/TBEV).

FIG. 9 is a series of graphs showing survival of mice inoculated IC with PIV-TBE and YF/TBE LAV constructs in a neurovirulence test (3.5 week old ICR mice; RV-WN/Hypr=PIV-WN/TBE(Hypr); CV-Hypr=YF/TBE(Hypr) LAV; CV-LGT=YF/LGT LAV).

FIG. 10 is a graph showing survival of mice inoculated IP with PIV-WN/TBE(Hypr) (RV-WN/Hypr), YF/TBE(Hypr) LAV (CV-Hypr), and YF/LGT LAV (CV-LGT) constructs and YF17D in a neuroinvasiveness test (3.5 week old ICR mice).

FIG. 11 is a series of graphs illustrating morbidity in mice measured by dynamics of body weight loss after TBE virus challenge, for groups immunized with s-PIV-TBE candidates (upper left panel), YF/TBE and YF/LGT chimeric viruses (upper right panel), and controls (YF 17D, human killed TBE vaccine, and mock; bottom panel).

FIG. 12 is a schematic representation of PIV constructs expressing rabies virus G protein, as well as illustration of packaging of the constructs to make pseudoinfectious virus and immunization.

FIG. 13 is a schematic representation of insertion designs resulting in viable/expressing constructs (exemplified by rabies G).

FIG. 14 is series of images showing immunofluorescence analysis and graphs showing growth curves of cells transfected with the indicated PIV-WN constructs (ΔC-Rabies G, ΔPrM-E-Rabies G, and ΔC-PrM-E-Rabies G).

FIG. 15 is a series of images showing immunofluorescence analysis of RabG expressed on the plasma membranes of Vero cells transfected with the indicated PIV constructs (ΔC-Rabies G, ΔPrM-E-Rabies G, and ΔC-PrM-E-Rabies G).

FIG. 16 is a schematic illustration of a PIV-WN-rabies G construct and a series of images showing that this construct spreads in helper cells, but not in naïve cells.

FIG. 17 is a series of graphs showing stability of the rabies G protein gene in PIV-WN vectors.

FIG. 18 is a set of images showing a comparison of spread of single-component vs. two-component PIV-WN-rabies G variants in Vero cells.

FIG. 19 is a set of immunofluorescence images showing expression of full-length RSV F protein (strain A2) by the AprM-E component of d-PIV-WN in helper cells after transfection.

FIG. 20 is a schematic representation of wild-type RSV F and RSV trF.

FIG. 21 is a schematic representation of three PIV(WN)-RSVtrF (A1 strain) constructs: ΔC-RSVtrF sPIV, ΔprME-RSVtrF dPIV helper, and ΔCprME-RSVtrF. Immunofluorescence of helper cells after transfection (Day 4) is also shown.

FIG. 22 is a series of images showing titration of WNAC-RSV trF PIV in Vero cells visualized by immunostaining.

FIG. 23 is an image showing a Western blot analysis of two ΔprME-RSVtrF stocks, 2 days post infection.

FIG. 24 is an image showing a Western blot analysis of Vero cells infected with the indicated amounts of VP2400, vFP2403, and PIV-F.

FIG. 25 is a set of graphs showing endpoint titers obtained using the indicated constructs and routes of administration in two sets of experiments (RSVi27 and RSVi32) indicating the anti-RSV-F IgG antibody titres obtained by ELISA. “F” represents vector with the F insert (truncated), while “e” represents the empty vector alone. “FI_RSV” is a formalin inactivated RSV virus, while “RSV” is a live RSV virus preparation.

FIG. 26 is a set of graphs showing serum neutralization titers obtained using the indicated constructs and routes of administration in two sets of experiments (RSVi27 and RSVi32). “F” represents vector with the F insert (truncated), while “e” represents the empty vector alone. “FI_RSV” is a formalin inactivated RSV virus, while “RSV” is a live RSV virus preparation (see FIG. 25).

### DETAILED DESCRIPTION OF THE INVENTION

The invention provides replication-defective or deficient pseudoinfectious virus (PIV) vectors including flavivirus sequences, which can be used in methods for inducing immunity against heterologous immunogens inserted into the vectors as well as, optionally, the vectors themselves. The invention also includes compositions including combinations of PIVs and/or Ply vectors, as described herein, and methods of using such compositions to induce immune responses against inserted immunogen sequences and/or sequences of the PIVs themselves. The focus of the invention is PIV vectors containing respiratory syncytial virus (RSV) immunogens, such as F or G protein immunogens, in one embodiment (see, e.g., truncated F protein, below). These vectors can be used in methods to prevent or treat RSV infection, and also in combination methods involving use of, for example, any of the other vectors described herein (such as vectors including immunogens of other pathogens and/or cancer, and/or allergy-related immunogens). The vectors, compositions, and methods of the invention are described further below.

### PIV Vectors

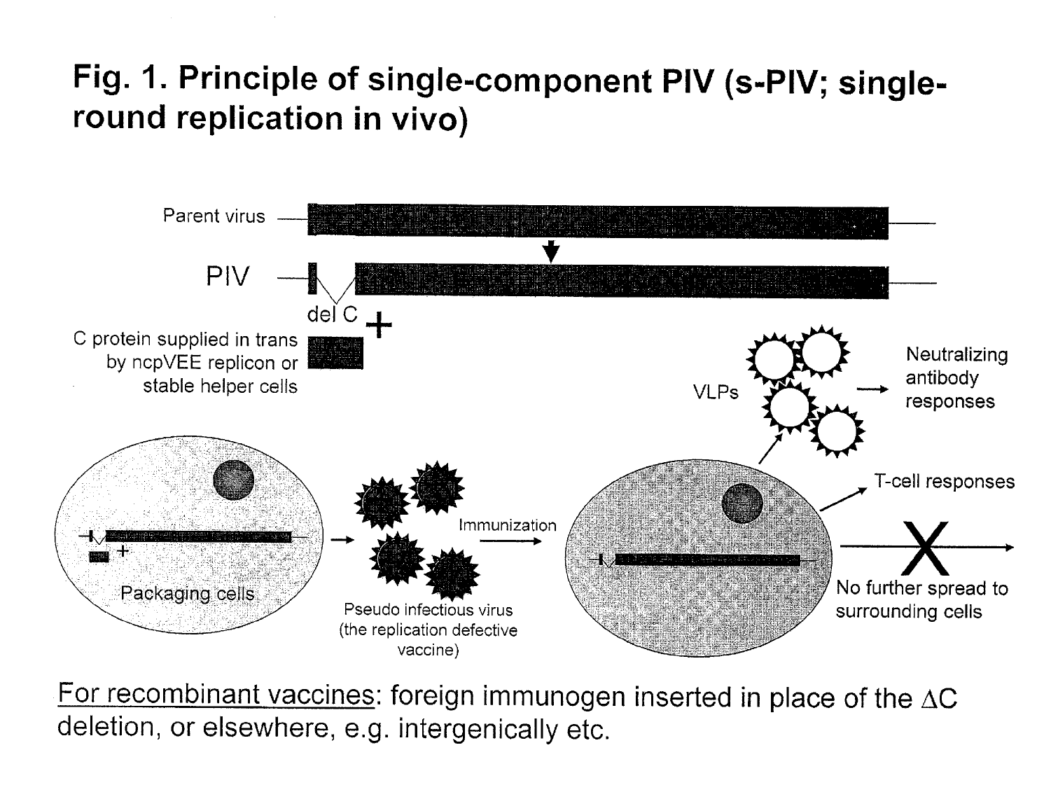

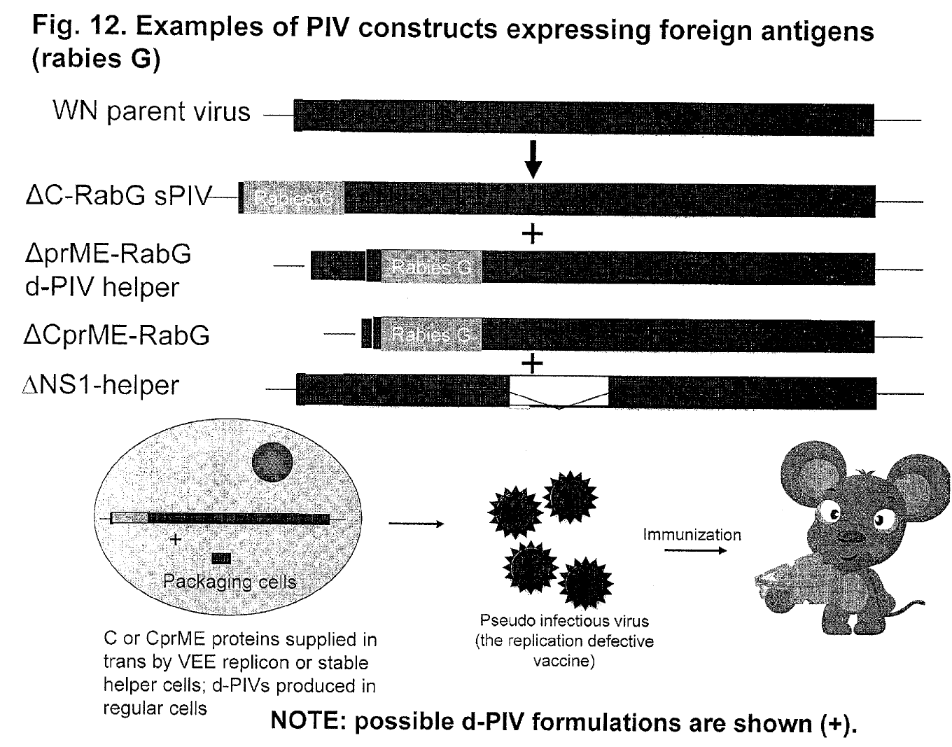

The PIV vectors of the invention can be based on the single- or two-component PIVs described above (also see WO 2007/098267 and WO 2008/137163). Thus, for example, in the case of single component PIVs, the PIV vectors and PIVs can include a genome including a large deletion in capsid protein encoding sequences and be produced in a complementing cell line that produces capsid protein in trans (single component; FIG. 1 and FIG. 12). According to this approach, most of the capsid-encoding region is deleted, which prevents the PIV genome from producing infectious progeny in normal cell lines (i.e., cell lines not expressing capsid sequences) and vaccinated subjects. The capsid deletion typically does not disrupt RNA sequences required for genome cyclization (i.e., the sequence encoding amino acids in the region of positions 1-26), and/or the prM sequence required for maturation of prM to M. In specific examples, the deleted sequences correspond to those encoding amino acids 26-100, 26-93, 31-100, or 31-93 of the C protein.

Single component PIV vectors and PIVs can be propagated in cell lines that express either C or a C-prM-E cassette, where they replicate to high levels. Exemplary cell lines that can be used for expression of single component PIV vectors and PIVs include BHK-21 (e.g., ATCC CCL-10), Vero (e.g., ATCC CCL-81), C7/10, and other cells of vertebrate or mosquito origin. The C or C-prM-E cassette can be expressed in such cells by use of a viral vector-derived replicon, such as an alphavirus replicon (e.g., a replicon based on Venezuelan Equine Encephalitis virus (VEEV), Sindbis virus, Semliki Forest virus (SFV), Eastern Equine Encephalitis virus (EEEV), Western Equine Encephalitis virus (WEEV), or Ross River virus). To decrease the possibility of productive recombination between the PIV vectors/PIVs and complementing sequences, the sequences in the replicons (encoding C, prM, and/or E) can include nucleotide mutations. For example, sequences encoding a complementing C protein can include an unnatural cyclization sequence. The mutations can result from codon optimization, which can provide an additional benefit with respect to PIV yield. Further, in the case of complementing cells expressing C protein sequences (and not a C-prM-E cassette), it may be beneficial to include an anchoring sequence at the carboxy terminus of the C protein including, for example, about 20 amino acids of prM (see, e.g., WO 2007/098267).

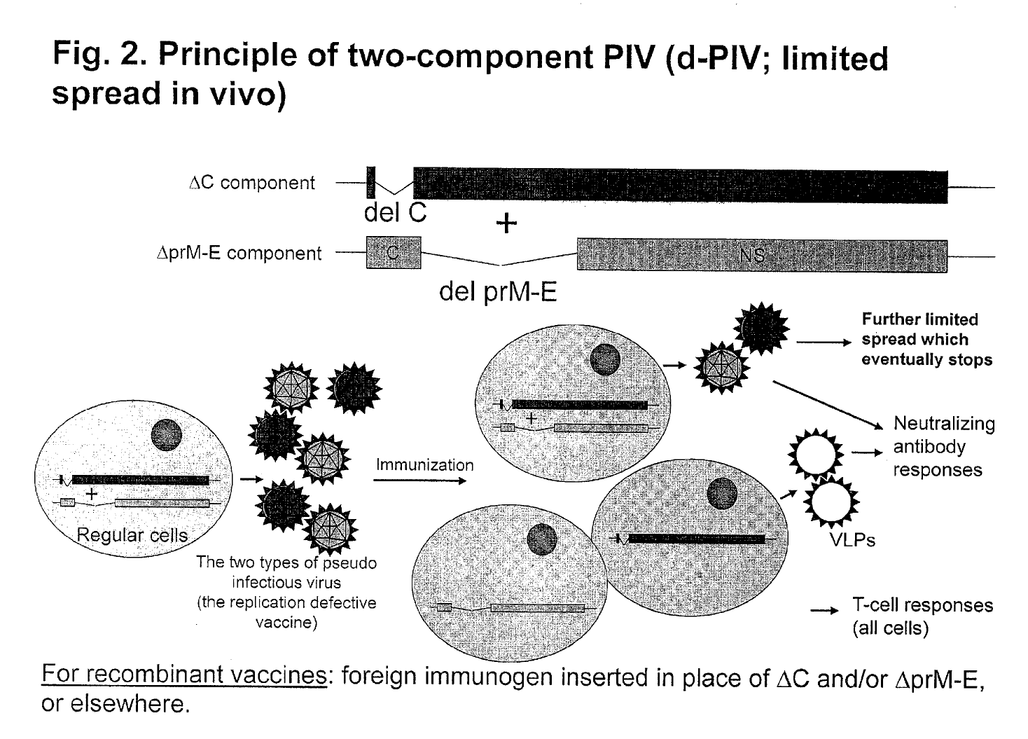

The PIV vectors and PIVs of the invention can also be based on the two-component genome technology described above. This technology employs two partial genome constructs, each of which is deficient in expression of at least one protein required for productive replication (capsid or prM/E) but, when present in the same cell, result in the production of all components necessary to make a PIV. Thus, in one example of the two-component genome technology, the first component includes a large deletion of C, as described above in reference to single component PIVs, and the second component includes a deletion of prM and E (FIG. 2 and FIG. 12). In another example, the first component includes a deletion of C, prM, and E, and the second component includes a deletion of NS1 (FIG. 12). Both components can include cis-acting promoter elements required for RNA replication and a complete set of non-structural proteins, which form the replicative enzyme complex. Thus, both defective genomes can include a 5′-untranslated region and at least about 60 nucleotides (Element 1) of the following, natural protein-coding sequence, which comprises an amino-terminal fragment of the capsid protein. This sequence can be followed by a protease cleavage sequence such as, for example, a ubiquitine or foot-and-mouth disease virus (FAMDV)-specific 2A protease sequence, which can be fused with either capsid or envelope (prM-E) coding sequences. Further, artificial, codon optimized sequences can be used to exclude the possibility of recombination between the two defective viral genomes, which could lead to formation of replication-competent viruses (see, e.g., WO 2008/137163). Use of the two-component genome approach does not require the development of cell lines expressing complementing genomes, such as the cells transformed with replicons, as discussed above in reference to the single component PIV approach. Exemplary cell lines that can be used in the two-component genome approach include Vero (e.g., ATCC CCL-81), BHK-21 (e.g., ATCC CCL-10), C7/10, and other cells of vertebrate or mosquito origin.

Additional examples of d-PIV approaches that can be used in the invention are based on use of complementing genomes including deletions in NS3 or NS5 sequences. A deletion in, e.g., NS1, NS3, or NS5 proteins can be used as long as several hundred amino acids in the ORF, removing the entire chosen protein sequence, or as short as 1 amino acid inactivating protein enzymatic activity (e.g., NS5 RNA polymerase activity, NS3 helicase activity, etc.). Alternatively, point amino acid changes (as few as 1 amino acid mutation, or optionally more mutations) can be introduced into any NS protein, inactivating enzymatic activity. In addition, several ΔNS deletions can be combined in one helper molecule. The same heterologous gene (such as an RSV F or G protein (e.g., truncated RSV F protein) gene), i.e., expressed by the first d-PIV component, can be expressed in place or in combination with the NS deletion(s) in the second component, increasing the amount of expressed immunogen. Notably, the insertion capacity of the helper will increase proportionally to the size of NS deletion(s). Alternatively, a different foreign immunogen(s) can be inserted in place of deletion(s) of the helper to produce multivalent vaccines.

Further, additional approaches that can be used in making PIV vectors and PIVs for use in the present invention are described, for example, in WO 99/28487, WO 03/046189, WO 2004/108936, US 2004/0265338, US 2007/0249032, and U.S. Pat. No. 7,332,322.

The PIV vectors of the invention can be comprised of sequences from a single flavivirus type (e.g., West Nile, tick-borne encephalitis (TBE, e.g., strain Hypr), Langat (LGT), yellow fever (e.g., YF17D), Japanese encephalitis, dengue (serotype 1-4), St. Louis encephalitis, Kunjin, Rocio encephalitis, Ilheus, Central European encephalitis, Siberian encephalitis, Russian Spring-Summer encephalitis, Kyasanur Forest Disease, Omsk Hemorrhagic fever, Louping ill, Powassan, Negishi, Absettarov, Hansalova, and Apoi viruses), or can comprise sequences from two or more different flaviviruses. Sequences of some strains of these viruses are readily available from generally accessible sequence databases; sequences of other strains can be easily determined by methods well known in the art. In the case of PIV vectors and PIVs including sequences of more than one flavivirus, the sequences can be those of a chimeric flavivirus, as described above (also see, e.g., U.S. Pat. No. 6,962,708; U.S. Pat. No. 6,696,281; and U.S. Pat. No. 6,184,024). In certain examples, the chimeras include pre-membrane and envelope sequences from one flavivirus (such as a flavivirus to which immunity may be desired), and capsid and non-structural sequences from a second, different flavivirus. In one specific example, the second flavivirus is a yellow fever virus, such as the vaccine strain YF17D. Other examples include the YF/WN, YF/TBE, YF/LGT, WN/TBE, and WN/LGT chimeras described below. Another example is an LGT/TBE chimera based on LGT virus backbone containing TBE virus prM-E proteins. A PIV vaccine based on this genetic background would have an advantage, because LGT replicates very efficiently in vitro and is highly attenuated and immunogenic for humans. Thus, a chimeric LGT/TBE PIV vaccine is expected to provide a robust specific immune response in humans against TBE, particularly due to inclusion of TBE prM-E genes.

Vectors of the invention can be based on PIV constructs or live, attenuated chimeric flaviviruses as described herein (in particular, YF/TBE, YF/LGT, WN/TBE, and WN/LGT; see below). Use of PIV constructs as vectors provides particular advantages in certain circumstances, because these constructs by necessity include large deletions, which render the constructs amenable to accommodation of insertions that are at least up to the size of the deleted sequences, without there being a loss in replication efficiency. Thus, PIV vectors in general can comprise very small insertions (e.g., in the range 6-10, 11-20, 21-100, 101-500, or more amino acid residues combined with the AC deletion or other deletions), as well as relatively large insertions or insertions of intermediate size (e.g., in the range 501-1000, 1001-1700, 1701-3000, or 3001-4000 or more residues). In contrast, in certain examples, it may be advantageous to express relatively short sequences in live attenuated viruses, particularly if the insertions are made in the absence of a corresponding deletion. Additional information concerning insertion sites that can be used in the invention is provided below. In addition, as discussed further below, expression of non-flavivirus immunogens in PIVs and chimeric flaviviruses of the invention can result in dual vaccines that elicit protective immunity against both a flavivirus vector virus pathogen and a target heterologous immunogen (e.g., RSV immunogens, such as those described herein).

As discussed above, the PIV vectors and PIVs of the invention can comprise sequences of chimeric flaviviruses, for example, chimeric flaviviruses including pre-membrane and envelope sequences of a first flavivirus (e.g., a flavivirus to which immunity is sought), and capsid and non-structural sequences of a second, different flavivirus, such as a yellow fever virus (e.g., YF17D; see above and also U.S. Pat. No. 6,962,708; U.S. Pat. No. 6,696,281; and U.S. Pat. No. 6,184,024). Further, chimeric flaviviruses (as well as non-chimeric flaviviruses, e.g., West Nile virus) used in the invention, used as a source for constructing PIVs, can optionally include one or more specific attenuating mutations (e.g., E protein mutations, prM protein mutations, deletions in the C protein, and/or deletions in the 3′UTR), such as any of those described in WO 2006/116182. For example, the C protein or 3′UTR deletions can be directly applied to YF/WN, YF/TBE, or YF/LGT chimeras Similar deletions can be designed and introduced in other chimeric LAV candidates such as based on LGT/TBE, WN/TBE, and WN/LGT genomes. With respect to E protein mutations, attenuating mutations similar to those described for YF/WN chimera in WO 2006/116182 can be designed, e.g., based on the knowledge of crystal structure of the E protein (Rey et al., Nature 375(6529):291-298, 1995), and employed. Further, additional examples of attenuating E protein mutations described for TBE virus and other flaviviruses are provided in Table 9. These can be similarly introduced into chimeric vaccine candidates.

The invention also provides new, particular chimeric flaviviruses, which can be used as a basis for the design of PIV vectors and PIVs, and as live attenuated chimeric flavivirus vectors. These chimeras include tick-borne encephalitis (TBE) virus or related prM-E sequences. Thus, the chimeras can include prM-E sequences from, for example, the Hypr strain of TBE or Langat (LGT) virus. Capsid and non-structural proteins of the chimeras can include those from yellow fever virus (e.g., YF17D) or West Nile virus (e.g., NY99).

A central feature of these exemplary YF/TBE, YF/LGT, WN/TBE, and WN/LGT chimeras is the signal sequence between the capsid and prM proteins. As is shown in the Examples, below, we have found that, in the case of YF-based PIV chimeras, it is advantageous to use a signal sequence comprising yellow fever and TBE sequences (see below). In one example, the signal sequence includes yellow fever sequences in the amino terminal region (e.g., SHDVLTVQFLIL) and TBE sequences in the carboxy terminal region (e.g., GMLGMTIA), resulting in the sequence SHDVLTVQFLILGMLGMTIA. We have also found that, in the case of WN-based PIV chimeras, it is advantageous to use a signal sequence comprising TBE sequences (e.g., GGTDWMSWLLVIGMLGMTIA). The invention thus includes YF/TBE, YF/LGT, WN/TBE, and WN/LGT chimeras, both PIVs and LAVs, which include the above-noted signal sequences, or variants thereof having, e.g., 1-8, 2-7, 3-6, or 4-5 amino acid substitutions, deletions, or insertions, which do not substantially interfere with processing at the signal sequence. In various examples, the substitutions are “conservative substitutions,” which are characterized by replacement of one amino acid residue with another, biologically similar residue. Examples of conservative substitutions include the substitution of one hydrophobic residue such as isoleucine, valine, leucine, or methionine for another, or the substitution of one polar residue for another, such as between arginine and lysine, between glutamic and aspartic acids, or between glutamine and asparagine and the like. Additional information concerning these chimeras is provided below, in the Examples.

### Insertion Sites

Sequences encoding immunogens can be inserted at one or more different sites within the vectors of the invention. Relatively short peptides can be delivered on the surface of PIV or LAV glycoproteins (e.g., prM, E, and/or NS1 proteins) and/or in the context of other proteins (to induce predominantly B-cell and T-cell responses, respectively). Other inserts, including larger portions of foreign proteins (e.g., certain RSV F or G protein sequences, as described herein), as well as complete proteins, can be expressed intergenically, at the N- and C-termini of the polyprotein, or bicistronically (e.g., within the ORF under an IRES or in the 3′UTR under an IRES; see, e.g., WO 02/102828, WO 2008/036146, WO 2008/094674, WO 2008/100464, WO 2008/115314, and below for further details). In PIV constructs, there is an additional option of inserting a foreign amino acid sequence directly in place of introduced deletion(s). Insertions can be made in, for example, AC, AprM-E, AC-prM-E, ANSI, ANS3, and ANS5. Thus, in one example, in the case of s-PIVs and the AC component of d-PIVs, immunogen-encoding sequences can be inserted in place of deleted capsid sequences. Immunogen-encoding sequences can also, optionally, be inserted in place of deleted prM-E sequences in the AprM-E component of d-PIVs. In another example, the sequences are inserted in place of or combined with deleted sequences in AC-prM-E constructs. Examples of such insertions are provided in the Examples section, below.

In the case of making insertions into PIV deletions, the insertions can be made with a few (e.g., 1, 2, 3, 4, or 5) additional vector-specific residues at the N- and/or C-termini of the foreign immunogen, if the sequence is simply fused in-frame (e.g., ˜20 first a.a. and a few last residues of the C protein if the sequence replaces the AC deletion), or without, if the foreign immunogen is flanked by appropriate elements well known in the field (e.g., viral protease cleavage sites; cellular protease cleavage sites, such as signalase, furin, etc.; autoprotease; termination codon; and/or IRES elements).

If a protein is expressed outside of the continuous viral open reading frame (ORF), e.g., if vector and non-vector sequences are separated by an internal ribosome entry site (IRES), cytoplasmic expression of the product can be achieved or the product can be directed towards the secretory pathway by using appropriate signal/anchor segments, as desired. If the protein is expressed within the vector ORF, important considerations include cleavage of the foreign protein from the nascent polyprotein sequence, and maintaining correct topology of the foreign protein and all viral proteins (to ensure vector viability) relative to the ER membrane, e.g., translocation of secreted proteins into the ER lumen, or keeping cytoplasmic proteins or membrane-associated proteins in the cytoplasm/in association with the ER membrane.

In more detail, the above-described approaches to making insertions can employ the use of, for instance, appropriate vector-derived, insert-derived, or unrelated signal and anchor sequencess included at the N and C termini of glycoprotein inserts. Standard autoproteases, such as FMDV 2A autoprotease (˜20 amino acids) or ubiquitin (gene ˜500 nt), or flanking viral NS2B/NS3 protease cleavage sites can be used to direct cleavage of an expressed product from a growing polypeptide chain, to release a foreign protein from a vector polyprotein, and to ensure viability of the construct. Optionally, growth of the polyprotein chain can be terminated by using a termination codon, e.g., following a foreign gene insert, and synthesis of the remaining proteins in the constructs can be re-initiated by incorporation of an IRES element, e.g., the encephalomyocarditis virus (EMCV) IRES commonly used in the field of RNA virus vectors. Viable recombinants can be recovered from helper cells (or regular cells for d-PIV versions). Optionally, backbone PIV sequences can be rearranged, e.g., if the latter results in more efficient expression of a foreign gene. For example, a gene rearrangement has been applied to TBE virus, in which the prM-E genes were moved to the 3′ end of the genome under the control of an IRES (Orlinger et al., J. Virol. 80:12197-12208, 2006). Translocation of prM-E or any other genes can be applied to PIV flavivirus vaccine candidates and expression vectors, according to the invention.

Additional details concerning different insertion sites that can be used in the invention are as follows (also see WO 02/102828, WO 2008/036146, WO 2008/094674, WO 2008/100464, WO 2008/115314, as noted above). Peptide sequences can be inserted within the envelope protein, which is the principle target for neutralizing antibodies. The sequences can be inserted into the envelope in, for example, positions corresponding to amino acid positions 59, 207, 231, 277, 287, 340, and/or 436 of the Japanese encephalitis virus envelope protein (see, e.g., WO 2008/115314 and WO 02/102828). To identify the corresponding loci in different flaviviruses, the flavivirus sequences are aligned with that of Japanese encephalitis virus. As there may not be an exact match, it should be understood that, in non-JE viruses, the site of insertion may vary by, for example, 1, 2, 3, 4, or 5 amino acids, in either direction. Further, given the identification of such sites as being permissive in JE, they can also vary in JE by, for example, 1, 2, 3, 4, or 5 amino acids, in either direction. Additional permissive sites can be identified using methods such as transposon mutagenesis (see, e.g., WO 02/102828 and WO 2008/036146). The insertions can be made at the indicated amino acids by insertion just C-terminal to the indicated amino acids (i.e., between amino acids 51-52, 207-208, 231-232, 277-278, 287-288, 340-341, and 436-437), or in place of short deletions (e.g., deletions of 1, 2, 3, 4, 5, 6, 7, or 8 amino acids) beginning at the indicated amino acids (or within 1-5 positions thereof, in either direction).

In addition to the envelope protein, insertions can be made into other virus proteins including, for example, the membrane/pre-membrane protein and NS1 (see, e.g., WO 2008/036146). For example, insertions can be made into a sequence preceding the capsid/pre-membrane cleavage site (at, e.g., −4, −2, or −1) or within the first 50 amino acids of the pre-membrane protein (e.g., at position 26), and/or between amino acids 236 and 237 of NS1 (or in regions surrounding the indicated sequences, as described above). In other examples, insertions can be made intergenically. For example, an insertion can be made between E and NS1 proteins and/or between NS2B and NS3 proteins (see, e.g., WO 2008/100464). In one example of an intergenic insertion, the inserted sequence can be fused with the C-terminus of the E protein of the vector, after the C-terminal signal/anchor sequence of the E protein, and the insertion can include a C-terminal anchor/signal sequence, which is fused with vector NS1 sequences. In another example of an intergenic insertion, the inserted sequences, with flanking protease cleavage sites (e.g., YF 17D cleavage sites), can be inserted into a unique restriction site introduced at the NS2B/NS3 junction (WO 2008/100464).

In other examples, a sequence can be inserted in the context of an internal ribosome entry site (IRES, e.g., an IRES derived from encephalomyocarditis virus; EMCV), as noted above, such as inserted in the 3′-untranslated region (WO 2008/094674). In one example of such a vector, employing, for example, yellow fever virus sequences, an IRES-immunogen cassette can be inserted into a multiple cloning site engineered into the 3′-untranslated region of the vector, e.g., in a deletion (e.g., a 136 nucleotide deletion in the case of a yellow fever virus-based example) after the polyprotein stop codon (WO 2008/094674).

Details concerning the insertion of rabies virus G protein and respiratory syncytial virus (RSV) F protein (including truncated F) into s-PIV and d-PIV vectors of the invention are provided below in Example 3. The information provided in Example 3 can be applied in the context of other vectors and immunogens described herein.

### Immunogens

PIVs (s-PIVs and d-PIVs) based on flavivirus sequences and live, attenuated chimeric flaviviruses (e.g., YF/WN, YF/TBE, YF/LGT, WN/TBE, and WN/LGT), as described above, can be used in the invention to deliver foreign (e.g., non-flavivirus) pathogen immunogens. The focus of the invention is the delivery of RSV immunogens, such as RSV fusion or F protein (or RSV G) immunogens (e.g., truncated F proteins; see below, for example the truncated F protein sequence in Example 3). PIVs and chimeric flavivirus vectors delivering a particular RSV immunogen can, optionally, be delivered with vectors delivering one or more other RSV immunogens, or one or more immunogens from another pathogen (e.g., viral, bacterial, fungal, and parasitic pathogens), one or more immunogens from cancer, and/or allergy-related immunogens. Specific, non-limiting examples of immunogens that can be delivered according to the invention are provided as follows.

As noted above, a central focus of the invention is delivery of the RSV proteins such as, in one embodiment, the RSV fusion or F glycoprotein and, in particular, truncated forms of this protein. The RSV F glycoprotein is one of the major immunogenic proteins of the virus. It is an envelope glycoprotein that mediates both fusion of the virus to the host cell membrane, and cell-to-cell spread of the virus. The amino acid sequence of the F protein is highly conserved among RSV subgroups A and B and is a cross-protective antigen.

RSV F protein comprises an extracellular region, a trans-membrane region, and a cytoplasmic tail region. A truncated protein delivered according to the invention can be, for example, one in which the trans-membrane and cytoplasmic tail regions of the F protein are absent (see, e.g., Example 3, below). Lack of expression of the trans-membrane region results in a secreted form of the RSV protein.

RSV F protein, as used herein, includes both full-length and truncated RSV fusion proteins, which may have the sequences described herein, or have variations in their amino acid sequences including naturally occurring in various strains of RSV and those introduced by PCR amplification of the encoding gene while retaining the immunogenic properties, a secreted form of the RSV F protein lacking a trans-membrane anchor and cytoplasmic tail, as well as fragments capable of generating antibodies which specifically react with RSV F protein and functional analogs. A first protein is a functional analog of a second protein if the first protein is immunologically related to and/or has the same function as the second protein. It may be for example, a fragment of the protein, or a substitution, addition, or deletion mutant thereof. The RSV F glycoprotein can be from, e.g., subgroup A or B (Wertz et al., Biotechnology 20:151-176, 1992).

In a further embodiment of the present invention, RSV G glycoprotein can be delivered. The G protein is a approximately 33 kDa protein and is heavily O-glycosylated, giving rise to a glycoprotein having a molecular weight of about 90 kDa (Levine, S., Kleiber-France, R., and Paradiso, P. R. (1987) J. Gen. Virol. 69, 2521-2524). The 298 amino acid residue RSV G protein belongs to the type II glycoproteins with the transmembrane domain (TM) located near the N-terminus (putative location: residues 38 to 66 underlined in Sequence Appendix 7. The RSV F and G proteins, or fragments or analogs thereof, can be from, for example, group A (e.g., A1 or A2) or B RSV.

Other examples of immunogens that can be delivered according to the invention are protective immunogens of the causative agent of Lyme disease (tick-borne spirochete Borrelia burgdorferi). In one example, PIVs including TBE/LGT sequences, as well as chimeric flaviviruses including TBE sequences (e.g., YF/TBE, YF/LGT, WN/TBE, LGT/TBE, and WN/LGT; in all instances where “TBE” is indicated, this includes the option of using the Hypr strain), can be used as vectors to deliver these immunogens. This combination, targeting both infectious agents (TBE and B. burgdorferi) is advantageous, because TBE and Lyme disease are both tick-borne diseases. The PIV approaches can be applied to chimeras (e.g., YF/TBE, YF/LGT, WN/TBE, or WN/LGT), according to the invention, as well as to non-chimeric TBE and LGT viruses. An exemplary immunogen from B. burgdorferi that can be used in the invention is OspA (Gipson et al., Vaccine 21:3875-3884, 2003). Optionally, to increase safety and/or immunogenicity, OspA can be mutated to reduce chances of autoimmune responses and/or to eliminate sites for unwanted post-translational modification in vertebrate animal cells, such as N-linked glycosylation, which may affect immunogenicity of the expression product. Mutations that decrease autoimmunity can include, e.g., those described by Willett et al., Proc. Natl. Acad. Sci. U.S.A. 101:1303-1308, 2004. In one example, FTK-OspA, a putative cross-reactive T cell epitope, Bb OspA₁₆₅₋₁₇₃ (YVLEGTLTA) is altered to resemble the corresponding peptide sequence of Borrelia afzelli (FTLEGKVAN). In FTK-OspA, the corresponding sequence is FTLEGKLTA.

The sequence of OspA is as follows:

| | |
|---|---|
| 1 | mkkyllgigl ilaliackqn vssldeknsv svdlpgemkv |
| | lvskeknkdg kydliatvdk |
| 61 | lelkgtsdkn ngsgvlegvk adkskvklti sddlgqttle |
| | vfkedgktlv skkvtskdks |
| 121 | steekfnekg evsekiitra dgtrleytgi ksdgsgkake |
| | vlkgyvlegt ltaekttlvv |
| 181 | kegtvtlskn isksgevsve lndtdssaat kktaawnsgt |
| | stltitvnsk ktkdlvftke |
| 241 | ntitvqqyds ngtklegsav eitkldeikn alk |

The full-length sequence and/or immunogenic fragments of the full-length sequence can be used. Exemplary fragments can include one or more of domains 1 (amino acids 34-41), 2 (amino acids 65-75), 3 (amino acids 190-220), and 4 (amino acids 250-270) (Jiang et al., Clin. Diag. Lab. Immun. 1(4):406-412, 1994). Thus, for example, a peptide comprising any one (or more) of the following sequences (which include sequence variations that can be included in the sequence listed above, in any combination) can be delivered: LPGE/GM/IK/T/GVL; GTSDKN/S/DNGSGV/T; N/H/EIS/P/L/A/SK/NSGEV/IS/TV/AE/ALN/DDT/SD/NS/TS/TA/Q/RATKKTA/GA/K/TWN/DS/AG/N/KT; SN/AGTK/NLEGS/N/K/TAVEIT/KK/TLD/KEI/LKN.

In addition to B. burgdorferi immunogens, tick saliva proteins, such as 64TRP, Isac, and Salp20, can be expressed, e.g., to generate a vaccine candidate of trivalent-specificity (TBE+Lyme disease+ticks). Alternatively, tick saliva proteins can be expressed instead of B. burgdorferi immunogens in TBE sequence-containing vectors. In addition, there are many other candidate tick saliva proteins that can be used for tick vector vaccine development according to the invention (Francischetti et al., Insect Biochem. Mol. Biol. 35:1142-1161, 2005). One or more of these immunogens can be expressed in s-PIV-TBE. However, d-PIV-TBE may also be selected, because of its large insertion capacity. In addition to PIV-TBE, other PIV vaccines can be used as vectors, e.g., to protect from Lyme disease and another flavivirus disease, such as West Nile virus. Expression of these immunogens can be evaluated in cell culture, and immunogenicity/protection examined in available animal models (e.g., as described in Gipson et al., Vaccine 21:3875-3884, 2003; Labuda et al., Pathog. 2(e27):0251-0259, 2006). Immunogens of other pathogens can be similarly expressed, in addition to Lyme disease and tick immunogens, with the purpose of making multivalent vaccine candidates. Exemplary tick saliva immunogens that can be used in the invention include the following:

64TRP (AF469170)
MKAFFVLSLL STAALTNAAR AGRLGSDLDT FGRVHGNLYA
GIERAGPRGY PGLTASIGGE VGARLGGRAG VGVSSYGYGY
PSWGYPYGGY GGYGGYGGYG GYDQGFGSAY GGYPGYYGYY
YPSGYGGGYG GSYGGSYGGS YTYPNVRASA GAAA
Isac (AF270496)
MRTAFTCALL AISFLGSPCS SSEDGLEQDT IVETTTQNLY
ERHYRNHSGL CGAQYRNSSH AEAVYNCTLN HLPPVVNATW
EGIRHRINKT IPQFVKLICN FTVAMPQEFY LVYMGSDGNS
DFEEDKESTG TDEDSNTGSS AAAKVTEALI IEAEENCTAH
ITGWTTETPT TLEPTTESQF EAIP
Salp20 (EU008559)
MRTALTCALL AISFLGSPCS SSEGGLEKDS RVETTTQNLY
ERYYRKHPGL CGAQYRNSSH AEAVYNCTLS LLPLSVNTTW
EGIRHRINKT IPEFVNLICN FTVAMPDQFY LVYMGSNGNS
YSEEDEDGKT GSSAAVQVTE QLIIQAEENC TAHITGWTTE
APTTLEPTTE TQFEAIS

Additional details concerning the TBE-related PIVs and LAVs are provided in Example 2, below.

Other PIV and LAV-vectored vaccines against other non-flavivirus pathogens, including vaccines having dual action, eliciting protective immunity against both flavivirus (as specified by the vector envelope proteins) and non-flavivirus pathogens (as specified by expressed immunologic determinant(s)) can also be used. These are similar to the example of PIV-TBE-Lyme disease-tick vector vaccines described above. As mentioned above, such dual-action vaccines can be developed against a broad range of pathogens by expression of immunogens from, for example, viral, bacterial, fungal, and parasitic pathogens, and immunogens associated with cancer and allergy. As specific non-limiting examples, we describe herein the design and biological properties of PIV vectored-rabies and -respiratory syncytial virus (RSV) vaccine candidates constructed by expression of rabies virus G protein or RSV F protein in place of or in combination with various deletions in one- and two-component PIV vectors (see Example 3, below).

As is demonstrated in the Examples, below, s-PIV constructs may be advantageously used to stably deliver relatively short foreign immunogens (similar to Lyme disease agent OspA protein and tick saliva proteins), because insertions are combined with a relatively short AC deletion. Two-component PIV vectors may be advantageously used to stably express relatively large immunogens, such as rabies G protein and RSV F, as the insertions in such vectors are combined with, for example, large AprM-E, AC-prM-E, and/or ANS1 deletions. Some of the d-PIV components can be manufactured and used as vaccines individually, for instance, the PIV-RSV F construct described below containing a AC-prM-E deletion. In this case, the vaccine induces an immune response (e.g., neutralizing antibodies) predominantly against the expressed protein, but not against the flavivirus vector virus pathogen. In other examples of the invention, dual immunity is obtained by having immunity induced both to vector and insert components. Additionally, because of the large insertion capacity of PIV vectors, and the option of using two-component genomes, PIV vectors offer the opportunity to target several non-flavivirus pathogens simultaneously, e.g., by expressing foreign immunogens from two different non-flavivirus pathogens in the two components of a d-PIV.

In addition to the RSV F or G protein, rabies G protein, Lyme disease protective immunogens, and tick saliva proteins, as examples of foreign immunogens described above, other foreign immunogens can be expressed to target respective diseases including, for example, influenza virus type A and B immunogens. In these examples, a few short epitopes and/or whole genes of viral particle proteins can be used, such as the M2, HA, and NA genes of influenza A, and/or the NB or BM2 genes of influenza B. Shorter fragments of M2, NB, and BM2, corresponding for instance to M2e, the extracellular fragment of M2, can also be used. In addition, fragments of the HA gene, including epitopes identified as HA0 (23 amino acids in length, corresponding to the cleavage site in HA) can be used. Specific examples of influenza-related sequences that can be used in the invention include PAKLLKERGFFGAIAGFLE (HA0), PAKLLKERGFFGAIAGFLEGSGC(HA0), NNATFNYTNVNPISHIRGS (NBe), MSLLTEVETPIRNEWGCRCNDSSD (M2e), MSLLTEVETPTRNEWECRCSDSSD (M2e), MSLLTEVETLTRNGWGCRCSDSSD (M2e), EVETPTRN (M2e), SLLTEVETPIRNEWGCRCNDSSD (M2e), and SLLTEVETPIRNEWGCR (M2e). Additional M2e sequences that can be used in the invention include sequences from the extracellular domain of BM2 protein of influenza B (consensus MLEPFQ, e.g., LEPFQILSISGC), and the M2e peptide from the H5N1 avian flu (MSLLTEVETLTRNGWGCRCSDSSD).

Other examples of pathogen immunogens that can be delivered in the vectors of the invention include codon-optimized SIV or HIV gag (55 kDa), gp120, gp160, SIV mac239-rev/tat/nef genes or analogs from HIV, and other HIV immunogens; immunogens from HPV viruses, such as HPV16, HPV18, etc., e.g., the capsid protein L1 which self-assembles into HPV-like particles, the capsid protein L2 or its immunodominant portions (e.g., amino acids 1-200, 1-88, or 17-36), the E6 and E7 proteins which are involved in transforming and immortalizing mammalian cells fused together and appropriately mutated (fusion of the two genes creates a fusion protein, referred to as E6E7Rb⁻, that is about 10-fold less capable of transforming fibroblasts, and mutations of the E7 component at 2 residues renders the resulting fusion protein mutant incapable of inducing transformation (Boursnell et al., Vaccine 14:1485-1494, 1996). Other immunogens include protective immunogens from HCV, CMV, HSV2, viruses, malaria parasite, Mycobacterium tuberculosis causing tuberculosis, C. difficile, and other nosocomial infections, that are known in the art, as well as fungal pathogens, cancer immunogens, and proteins associated with allergy that can be used as vaccine targets.

Foreign immunogen inserts of the invention, such as RSV immunogens as described herein, can be modified in various ways. For instance, codon optimization is used to increase the level of expression and eliminate long repeats in nucleotide sequences to increase insert stability in the RNA genome of PIV vectors. Further, the genes can be truncated at N- and/or C-termini, or by internal deletion(s), or modified by specific amino acid changes to increase visibility to the immune system and immunogenicity. Immunogenicity can be increased by chimerization of proteins with immunostimulatory moieties well known in the art, such as TLR agonists, stimulatory cytokines, components of complement, heat-shock proteins, etc. (e.g., reviewed in “Immunopotentiators in Modern Vaccines,” Schijns and O'Hagan Eds., 2006, Elsevier Academic Press: Amsterdam, Boston).

With respect to construction of dual vaccines against rabies and other flavivirus diseases, other combinations, such as TBE+rabies, YF+rabies, etc., can be of interest both for human and veterinary use in corresponding geographical regions, and thus can be similarly generated. Possible designs of expression constructs are not limited to those described herein. For example deletions and insertions can be modified, genetic elements can be rearranged, or other genetic elements (e.g. non-flavivirus, non-rabies signals for secretion, intracellular transport determinants, inclusion of or fusion with immunostimulatory moieties such as cytokines, TLR agonists such as flagellin, multimerization components such as leucine zipper, and peptides that increase the period of protein circulation in the blood) can be used to facilitate antigen presentation and increase immunogenicity. Further, such designs can be applied to s-PIV and d-PIV vaccine candidates based on vector genomes of other flaviviruses, and expressing immunogens of other pathogens, e.g., including but not limited to pathogens described in elsewhere herein.

Other examples of PIV and LAV vectors of the invention including combination vaccines such as DEN+Chikungunya virus (CHIKV) and YF+CHIKV. CHIKV, an alphavirus, is endemic in Africa, South East Asia, Indian subcontinent and the Islands, and the Pacific Islands and shares ecological/geographical niches with YF and DEN1-4. It causes serious disease primarily associated with severe pain (arthritis, other symptoms similar to DEN) and long-lasting sequelae in the majority of patients (Simon et al., Med. Clin. North Am. 92:1323-1343, 2008; Seneviratne et al., J. Travel Med. 14:320-325, 2007). Other examples of PIV and LAV vectors of the invention include YF+Ebola or DEN+Ebola, which co-circulate in Africa.

Immunogens for the above-noted non-flavivirus pathogens, sequences of which are well known in the art, may include glycoprotein B or a pp 65/1E1 fusion protein of CMV (Reap et al., Vaccine 25(42):7441-7449, 2007; and references therein), several TB proteins (reviewed in Skeiky et al., Nat. Rev. Microbiol. 4(6):469-476, 2006), malaria parasite antigens such as RTS,S (a pre-erythrocytic circumsporozoite protein, CSP) and others (e.g., reviewed in Li et al., Vaccine 25(14):2567-2574, 2007), CHIKV envelope proteins E1 and E2 (or the C-E2-E1, E2-E1 cassettes), HCV structural proteins C-E1-E2 forming VLPs (Ezelle et al., J. Virol. 76(23):12325-12334, 2002) or other proteins to induce T=cell responses, Ebola virus glycoprotein GP (Yang et al., Virology 377(2):255-264, 2008).

In addition to the immunogens described above, the vectors described herein may include one or more immunogen(s) derived from or that direct an immune response against one or more viruses (e.g., viral target antigen(s)) including, for example, a dsDNA virus (e.g., adenovirus, herpesvirus, epstein-barr virus, herpes simplex type 1, herpes simplex type 2, human herpes virus simplex type 8, human cytomegalovirus, varicella-zoster virus, poxvirus); ssDNA virus (e.g., parvovirus, papillomavirus (e.g., E1, E2, E3, E4, E5, E6, E7, E8, BPV1, BPV2, BPV3, BPV4, BPV5, and BPV6 (In Papillomavirus and Human Cancer, edited by H. Pfister (CRC Press, Inc. 1990)); Lancaster et al., Cancer Metast. Rev. pp. 6653-6664, 1987; Pfister et al., Adv. Cancer Res. 48:113-147, 1987)); dsRNA viruses (e.g., reovirus); (+)ssRNA viruses (e.g., picornavirus, coxsackie virus, hepatitis A virus, poliovirus, togavirus, rubella virus, flavivirus, hepatitis C virus, yellow fever virus, dengue virus, west Nile virus); (−)ssRNA viruses (e.g., orthomyxovirus, influenza virus, rhabdovirus, paramyxovirus, measles virus, mumps virus, parainfluenza virus, rhabdovirus, rabies virus); ssRNA-RT viruses (e.g., retrovirus, human immunodeficiency virus (HIV)); and dsDNA-RT viruses (e.g. hepadnavirus, hepatitis B). Immunogens may also be derived from other viruses not listed above but available to those of skill in the art.

With respect to HIV, immunogens may be selected from any HIV isolate. As is well-known in the art, HIV isolates are now classified into discrete genetic subtypes. HIV-1 is known to comprise at least ten subtypes (A, B, C, D, E, F, G, H, J, and K). HIV-2 is known to include at least five subtypes (A, B, C, D, and E). Subtype B has been associated with the HIV epidemic in homosexual men and intravenous drug users worldwide. Most HIV-1 immunogens, laboratory adapted isolates, reagents and mapped epitopes belong to subtype B. In sub-Saharan Africa, India, and China, areas where the incidence of new HIV infections is high, HIV-1 subtype B accounts for only a small minority of infections, and subtype HIV-1 C appears to be the most common infecting subtype. Thus, in certain embodiments, it may be desirable to select immunogens from HIV-1 subtypes B and/or C. It may be desirable to include immunogens from multiple HIV subtypes (e.g., HIV-1 subtypes B and C, HIV-2 subtypes A and B, or a combination of and HIV-2 subtypes) in a single immunological composition. Suitable HIV immunogens include ENV, GAG, POL, NEF, as well as variants, derivatives, and fusion proteins thereof, for example.

Immunogens may also be derived from or direct an immune response against one or more bacterial species (spp.) (e.g., bacterial target antigen(s)) including, for example, Bacillus spp. (e.g., Bacillus anthracis), Bordetella spp. (e.g., Bordetella pertussis), Borrelia spp. (e.g., Borrelia burgdorferi), Brucella spp. (e.g., Brucella abortus, Brucella canis, Brucella melitensis, Brucella suis), Campylobacter spp. (e.g., Campylobacter jejuni), Chlamydia spp. (e.g., Chlamydia pneumoniae, Chlamydia psittaci, Chlamydia trachomatis), Clostridium spp. (e.g., Clostridium botulinum, Clostridium difficile, Clostridium perfringens, Clostridium tetani), Corynebacterium spp. (e.g., Corynebacterium diptheriae), Enterococcus spp. (e.g., Enterococcus faecalis, enterococcus faecum), Escherichia spp. (e.g., Escherichia coli), Francisella spp. (e.g., Francisella tularensis), Haemophilus spp. (e.g., Haemophilus influenza), Helicobacter spp. (e.g., Helicobacter pylori), Legionella spp. (e.g., Legionella pneumophila), Leptospira spp. (e.g., Leptospira interrogans), Listeria spp. (e.g., Listeria monocytogenes), Mycobacterium spp. (e.g., Mycobacterium leprae, Mycobacterium tuberculosis), Mycoplasma spp. (e.g., Mycoplasma pneumoniae), Neisseria spp. (e.g., Neisseria gonorrhea, Neisseria meningitidis), Pseudomonas spp. (e.g., Pseudomonas aeruginosa), Rickettsia spp. (e.g., Rickettsia rickettsii), Salmonella spp. (e.g., Salmonella typhi, Salmonella typhinurium), Shigella spp. (e.g., Shigella sonnei), Staphylococcus spp. (e.g., Staphylococcus aureus, Staphylococcus epidermidis, Staphylococcus saprophyticus, coagulase negative staphylococcus (e.g., U.S. Pat. No. 7,473,762)), Streptococcus spp. (e.g., Streptococcus agalactiae, Streptococcus pneumoniae, Streptococcus pyrogenes), Treponema spp. (e.g., Treponema pallidum), Vibrio spp. (e.g., Vibrio cholerae), and Yersinia spp. (Yersinia pestis). Immunogens may also be derived from or direct the immune response against other bacterial species not listed above but available to those of skill in the art.

Immunogens may also be derived from or direct an immune response against one or more parasitic organisms (spp.) (e.g., parasite target antigen(s)) including, for example, Ancylostoma spp. (e.g., A. duodenale), Anisakis spp., Ascaris lumbricoides, Balantidium coli, Cestoda spp., Cimicidae spp., Clonorchis sinensis, Dicrocoelium dendriticum, Dicrocoelium hospes, Diphyllobothrium latum, Dracunculus spp., Echinococcus spp. (e.g., E. granulosus, E. multilocularis), Entamoeba histolytica, Enterobius vermicularis, Fasciola spp. (e.g., F. hepatica, F. magna, F. gigantica, F. jacksoni), Fasciolopsis buski, Giardia spp. (Giardia lamblia), Gnathostoma spp., Hymenolepis spp. (e.g., H. nana, H. diminuta), Leishmania spp., Loa boa, Metorchis spp. (M. conjunctus, M. albidus), Necator americanus, Oestroidea spp. (e.g., botfly), Onchocercidae spp., Opisthorchis spp. (e.g., O. viverrini, O. felineus, O. guayaquilensis, and O. noverca), Plasmodium spp. (e.g., P. falciparum), Protofasciola robusta, Parafasciolopsis fasciomorphae, Paragonimus westermani, Schistosoma spp. (e.g., S. mansoni, S. japonicum, S. mekongi, S. haematobium), Spirometra erinaceieuropaei, Strongyloides stercoralis, Taenia spp. (e.g., T. saginata, T. solium), Toxocara spp. (e.g., T. canis, T. cati), Toxoplasma spp. (e.g., T. gondii), Trichobilharzia regenti, Trichinella spiralis, Trichuris trichiura, Trombiculidae spp., Trypanosoma spp., Tunga penetrans, and/or Wuchereria bancrofti. Immunogens may also be derived from or direct the immune response against other parasitic organisms not listed above but available to those of skill in the art.

Immunogens may be derived from or direct the immune response against tumor target antigens (e.g., tumor target antigens). The term tumor target antigen (TA) may include both tumor-associated antigens (TAAs) and tumor-specific antigens (TSAs), where a cancerous cell is the source of the antigen. A TA may be an antigen that is expressed on the surface of a tumor cell in higher amounts than is observed on normal cells or an antigen that is expressed on normal cells during fetal development. A TSA is typically an antigen that is unique to tumor cells and is not expressed on normal cells. TAs are typically classified into five categories according to their expression pattern, function, or genetic origin: cancer-testis (CT) antigens (i.e., MAGE, NY-ESO-1); melanocyte differentiation antigens (e.g., Melan A/MART-1, tyrosinase, gp100); mutational antigens (e.g., MUM-1, p53, CDK-4); overexpressed ‘self’ antigens (e.g., HER-2/neu, p53); and viral antigens (e.g., HPV, EBV). Suitable TAs include, for example, gp100 (Cox et al., Science 264:716-719, 1994), MART-1/Melan A (Kawakami et al., J. Exp. Med., 180:347-352, 1994), gp75 (TRP-1) (Wang et al., J. Exp. Med., 186:1131-1140, 1996), tyrosinase (Wolfel et al., Eur. J. Immunol., 24:759-764, 1994), NY-ESO-1 (WO 98/14464; WO 99/18206), melanoma proteoglycan (Hellstrom et al., J. Immunol., 130:1467-1472, 1983), MAGE family antigens (e.gl, MAGE-1, 2, 3, 4, 6, and 12; Van der Bruggen et al., Science 254:1643-1647, 1991; U.S. Pat. No. 6,235,525), BAGE family antigens (Boel et al., Immunity 2:167-175, 1995), GAGE family antigens (e.g., GAGE-1,2; Van den Eynde et al., J. Exp. Med. 182:689-698, 1995; U.S. Pat. No. 6,013,765), RAGE family antigens (e.g., RAGE-1; Gaugler et al., Immunogenetics 44:323-330, 1996; U.S. Pat. No. 5,939,526), N-acetylglucosaminyltransferase-V (Guilloux et al., J. Exp. Med. 183:1173-1183, 1996), p15 (Robbins et al., J. Immunol. 154:5944-5950, 1995), β-catenin (Robbins et al., J. Exp. Med., 183:1185-1192, 1996), MUM-1 (Coulie et al., Proc. Natl. Acad. Sci. U.S.A. 92:7976-7980, 1995), cyclin dependent kinase-4 (CDK4) (Wolfel et al., Science 269:1281-1284, 1995), p21-ras (Fossum et al., Int. J. Cancer 56:40-45, 1994), BCR-abd (Bocchia et al., Blood 85:2680-2684, 1995), p53 (Theobald et al., Proc. Natl. Acad. Sci. U.S.A. 92:11993-11997, 1995), p185 HER2/neu (erb-B1; Fisk et al., J. Exp. Med., 181:2109-2117, 1995), epidermal growth factor receptor (EGFR) (Harris et al., Breast Cancer Res. Treat, 29:1-2, 1994), carcinoembryonic antigens (CEA) (Kwong et al., J. Natl. Cancer Inst., 85:982-990, 1995) U.S. Pat. Nos. 5,756,103; 5,274,087; 5,571,710; 6,071,716; 5,698,530; 6,045,802; EP 263933; EP 346710; and EP 784483; carcinoma-associated mutated mucins (e.g., MUC-1 gene products; Jerome et al., J. Immunol., 151:1654-1662, 1993); EBNA gene products of EBV (e.g., EBNA-1; Rickinson et al., Cancer Surveys 13:53-80, 1992); E7, E6 proteins of human papillomavirus (Ressing et al., J. Immunol. 154:5934-5943, 1995); prostate specific antigen (PSA; Xue et al., The Prostate 30:73-78, 1997); prostate specific membrane antigen (PSMA; Israeli et al., Cancer Res. 54:1807-1811, 1994); idiotypic epitopes or antigens, for example, immunoglobulin idiotypes or T cell receptor idiotypes (Chen et al., J. Immunol. 153:4775-4787, 1994); KSA (U.S. Pat. No. 5,348,887), kinesin 2 (Dietz, et al., Biochem. Biophys. Res. Commun. 275(3):731-738, 2000), HIP-55, TGFβ-1 anti-apoptotic factor (Toomey et al., Br. J. Biomed. Sci. 58(3):177-183, 2001), tumor protein D52 (Bryne et al., Genomics 35:523-532, 1996), H1FT, NY-BR-1 (WO 01/47959), NY-BR-62, NY-BR-75, NY-BR-85, NY-BR-87, and NY-BR-96 (Scanlan, M. Serologic and Bioinformatic Approaches to the Identification of Human Tumor Antigens, in Cancer Vaccines 2000, Cancer Research Institute, New York, N.Y.), and/or pancreatic cancer antigens (e.g., SEQ ID NOs: 1-288 of U.S. Pat. No. 7,473,531). Immunogens may also be derived from or direct the immune response against include TAs not listed above but available to one of skill in the art.

In addition to the specific immunogen sequences listed above, the invention also includes the use of analogs of the sequences. Such analogs include sequences that are, for example, at least 80%, 90%, 95%, or 99% identical to the reference sequences, or fragments thereof. The analogs can include one or more substitutions or deletions, e.g., substitutions of conservative amino acids as described herein. The analogs also include fragments of the reference sequences that include, for example, one or more immunogenic epitopes of the sequences. Further, the analogs include truncations or expansions of the sequences (e.g., insertion of additionaUrepeat immunodominant/helper epitopes) by, e.g., 1, 2, 3, 4, 5, 6, 7, 8, 9, 10, 11-20, etc., amino acids on either or both ends. Truncation may remove immunologically unimportant or interfering sequences, e.g., within known structural/immunologic domains, or between domains; or whole undesired domains can be deleted; such modifications can be in the ranges 21-30, 31-50, 51-100, 101-400, etc. amino acids. The ranges also include, e.g., 20-400, 30-100, and 50-100 amino acids.

### Cocktails

The invention also includes compositions including mixtures of two or more PIVs and/or PIV vectors, as described herein. As discussed above, use of such mixtures or cocktails may be particularly advantageous when induction of immunity to more than one immunogen and/or pathogen is desired. This may be useful, for example, in vaccination against different flaviviruses that may be endemic to the region in which the vaccine recipient resides. This may also be useful in the context of administration of multiple immunogens against the same target.

Non-limiting examples of PIV cocktails included in the invention are those including PIV-JE+PIV-DEN, and PIV-YF+PIV-DEN. In both of these examples, the PIVs for either or both components can be single or dual component PIVs, as described above. In addition, in the case of the Ply-DEN, the PIV can include sequences of just one dengue serotype selected from the group consisting of dengue serotypes 1-4, or the cocktail can include PIVs expressing sequences from two, three, or all four of the serotypes. Further, the TBE/Borrelia burgdorferi/tick saliva protein (e.g., 64TRP, Isac, Salp20) vaccines described herein can be based on including the different immunogens within a single PIV or live attenuated flavivirus, or can be based on mixtures of PIVs (or LAVs), which each include one or more of the immunogens. The cocktails of the invention can be formulated as such or can be mixed just prior to administration.

### Use, Formulation, and Administration

The invention includes the PIV and LAV vectors, as well as corresponding nucleic acid molecules, pharmaceutical or vaccine compositions, and methods of their use and preparation. The PIV and LAV vectors of the invention can be used, for example, in vaccination methods to induce an immune response to RSV and/or the flavivirus vector, and/or another expressed immunogen, as described herein. These methods can be prophylactic, in which case they are carried out on subjects (e.g., human subjects or other mammalian subjects) not having, but at risk of developing infection or disease caused by RSV or flavivirus and/or a pathogen from which another expressed immunogen is derived. Such methods include instances in which a subject becomes infected by RSV, but is able to ward off the infection and significant symptomatic disease, because of the treatment according to the invention. The methods can also be therapeutic, in which they are carried out on subjects already having an infection by one or more of the relevant pathogens, such as RSV. Such methods include the amelioration of one or more symptoms of the infection, whether partial or complete. Further, the viruses and vectors can be used individually or in combination with one another or other vaccines. The subjects treated according to the methods of the invention include humans, as well as non-human mammals (e.g., livestock, such as, cattle, pigs, horses, sheep, and goats, and domestic animals, including dogs and cats). Of particular interest with respect to vaccination against RSV are infants and young children, including pre-mature infants, as well as middle aged and elderly people. Thus, for example, human patients age 1 day to five years (e.g., 2 months to 3 years, or 4 months to two years), or age 50 to 65 and above.

Formulation of the PIV and LAV vectors of the invention can be carried out using methods that are standard in the art. Numerous pharmaceutically acceptable solutions for use in vaccine preparation are well known and can readily be adapted for use in the present invention by those of skill in this art (see, e.g., Remington's Pharmaceutical Sciences (18^{th} edition), ed. A. Gennaro, 1990, Mack Publishing Co., Easton, Pa.). In two specific examples, the PIV vectors, PIVs, LAV vectors, and LAVs are formulated in Minimum Essential Medium Earle's Salt (MEME) containing 7.5% lactose and 2.5% human serum albumin or MEME containing 10% sorbitol. However, the PIV and LAV vectors can simply be diluted in a physiologically acceptable solution, such as sterile saline or sterile buffered saline.

The PIV and LAV vectors of the invention can be administered using methods that are well known in the art, and appropriate amounts of the viruses and vectors to be administered can readily be determined by those of skill in the art. What is determined to be an appropriate amount of virus to administer can be determined by consideration of factors such as, e.g., the size and general health of the subject to whom the virus is to be administered. For example, in the case of live, attenuated viruses of the invention, the viruses can be formulated as sterile aqueous solutions containing between 10² and 10⁸, e.g., 10³ to 10⁷, infectious units (e.g., plaque-forming units or tissue culture infectious doses) in a dose volume of 0.1 to 1.0 ml. PIVs can be administered at similar doses and in similar volumes; PIV titers however are usually measured in, e.g., focus-forming units determined by immunostaining of foci, as these defective constructs tend not to form virus-like plaques. Doses can range between 10² and 10⁸ FFU and administered in volumes of 0.1 to 1.0 ml.

All viruses and vectors of the invention can be administered by, for example, intradermal, subcutaneous, intramuscular, intraperitoneal, intranasal (e.g., by inhalation or nose drops), intravenous, or oral routes. In specific examples, dendritic cells are targeted by intradermal or transcutaneous administration, by use of, for example, microneedles or microabrasion devices. Further, the vaccines of the invention can be administered in a single dose or, optionally, administration can involve the use of a priming dose followed by a booster dose that is administered, e.g., 2-6 months later, as determined to be appropriate by those of skill in the art. Optionally, PIV vaccines can be administered via DNA or RNA immunization using methods known to those skilled in the art (Chang et al., Nat. Biotechnol. 26:571-577, 2008; Kofler et al., Proc. Natl. Acad. Sci. U.S.A. 101:1951-1956, 2004).

Optionally, adjuvants that are known to those skilled in the art can be used in the administration of the viruses and vectors of the invention. Adjuvants that can be used to enhance the immunogenicity of the viruses include, for example, liposomal formulations, synthetic adjuvants, such as (e.g., QS21), muramyl dipeptide, monophosphoryl lipid A, polyphosphazine, CpG oligonucleotides, or other molecules that appear to work by activating Toll-like Receptor (TLR) molecules on the surface of cells or on nuclear membranes within cells. Although these adjuvants are typically used to enhance immune responses to inactivated vaccines, they can also be used with live or replication-defective vaccines. Both agonists of TLRs or antagonists may be useful in the case of live or replication-defective vaccines. The vaccine candidates can be designed to express TLR agonists. In the case of a virus delivered via a mucosal route, for example, orally, mucosal adjuvants such as the heat-labile toxin of E. coli (LT) or mutant derivations of LT can be used as adjuvants. In addition, genes encoding cytokines that have adjuvant activities can be inserted into the vaccine candidates. Thus, genes encoding desired cytokines, such as GM-CSF, IL-2, IL-12, IL-13, IL-5, etc., can be inserted together with foreign immunogen genes to produce a vaccine that results in enhanced immune responses, or to modulate immunity directed more specifically towards cellular, humoral, or mucosal responses (e.g., reviewed in “Immunopotentiators in Modem Vaccines”, Schijns and O'Hagan Eds., 2006, Elsevier Academic Press: Amsterdam, Boston, etc.). Optionally, a patch containing a layer of an appropriate toxin-derived adjuvant, can be applied over the injection site. Toxin promotes local inflammation attracting lymphocytes, which leads to a more robust immune response.

### EXAMPLES

Additional details concerning the invention are provided in the Examples, below. In the Examples, experiments are described in which PIVs based on WN, JE, and YF viruses (see, e.g., WO 2007/098267 and WO 2008/137163) were tested. Firstly, we demonstrated that the constructs are significantly more attenuated in a sensitive suckling mouse neurovirulence model (zero mortality at all tested doses) as compared to available LAV controls (YF 17D, YF/JE LAV, and YF/WN LAV). We demonstrated for the first time that d-PIV constructs were avirulent in this model and thus that two-component PIVs do not undergo uncontrolled (unlimited) spread in vivo and cannot cause clinical signs. Secondly, we performed comparisons of the immunogenicity and efficacy of the PIVs and the LAVs, and demonstrated that PIV vaccines can induce immune response comparable to LAVs and be equally efficacious (e.g., as observed for PIV-WN and YF/WN LAV pair of vaccines). In one pair examined, YF 17D LAV was significantly more immunogenic than PIV-YF. Thus, production of VLPs can vary between different, similarly designed PIV constructs. Specifically, we propose that PIV-YF does not generate a large amount of YF VLPs compared to PIV-WN (WN VLPs), and that increased production of VLPs can be achieved by genetic modifications at the C/prM junction in suboptimal PIV constructs. Specifically, the C/prM junction is an important location in the flavivirus polyprotein orchestrating the formation of viral envelope and synthesis of viral proteins (Yamshchikov and Compans, Virology 192:38-51, 1993; Amberg and Rice, J. Virol. 73:8083-8094, 1999; Stocks and Lobigs, J. Virol. 72:2141-2149, 1998). We propose that secretion of VLPs in PIV infected cells (in contrast to production of viral particles in whole viruses) can be increased by uncoupling of the viral protease and signalase cleavages at the junction, or use of a strong heterologous signal peptide (tPA, etc.) in place of the signal for prM, or by mutagenesis of the signal for prM. The efficiency of signalase cleavage at the C/prM junction of flaviviruses is low (Stocks and Lobigs, J. Virol. 72:2141-2149, 1998), e.g., as predicted by SignalP 3.0 on-line program. It is expected that more efficient cleavage efficiency can be achieved by analysis of specific amino acid substitutions near the cleavage site with SignalP 3.0 (e.g., as described in application WO 2008/100464), followed by incorporation of chosen mutation(s) into PIV genomes, recovery of PIV progeny and measuring VLP secretion. Non-flavivirus signals are inserted by methods standard in the art. Uncoupling between the viral protease and signalase cleavages can be achieved by ablating the viral cleavage site by any non-conservative mutation (e.g., RRS in YF17D C to RRA or GRS or RSS, etc.), or deletion of the entire site or some of its 3 residues. If necessary, formation of free N-terminus of the signal of foreign protein can be achieved by using such elements as autoprotease, or termination codon followed by an IRES. Alternatively, the native AUG initiation codon of C can be ablated (in constructs where C protein sequence is unnecessary, e.g., AC PIV) and AUG placed in front of foreign gene. Optimization of vector signal can be performed by random mutagenesis, e.g., by insertion of synthetic randomized sequence followed by identification of viable PIV variants with increased VLP secretion.

We also discovered that PIV constructs were substantially more immunogenic in hamsters when administered by the IP route, as compared to the subcutaneous route. We concluded that this was most likely due to better targeting of antigen presenting cells in lymphoid tissues, which are abundant in the abdomen, but not abundant in tissues underlying the skin. Based on these observations, we concluded that efficient targeting of PIVs to dendritic cells, abundant in the skin, can be achieved by cutaneous inoculation, e.g., via skin microabrasion or intradermal injection using microneedles (Dean et al., Hum Vaccin. 1:106-111, 2005).

Further, we have carried out experiments to show the feasibility of administering mixtures, or cocktails, of different PIVs, such as those described herein (e.g., JE+DEN and YF+DEN). In order to administer cocktails, it is important to verify that there is no interference between co-administered components, and that a balanced immune response is induced. Several PIV mixtures were used to immunize rodents and immune responses were compared to PIV constructs administered individually. No interference was observed in mixtures, and thus cocktail PIV vaccines are feasible. Such formulations may be of particular significance in geographical regions where different flaviviruses co-circulate. This could be also used to simultaneously administer several PIV-based vaccines against non-flavivirus pathogens.

Further, we have demonstrated that no neutralizing antibody response is induced against packaging envelope after at least two doses of PIV (and thus antibodies are elicited against VLPs secreted from infected cells). This was demonstrated using the helper (AprM-E) component of a d-PIV (see in FIG. 2) packaged individually, or by measuring neutralizing antibodies to heterologous packaging envelopes (e.g., to the WN envelope used to package PIV-JE in helper cells providing WN-specific C-prM-E proteins in trans). The latter observations support sequential use of different PIV vaccines manufactured in a universal helper packaging cells line, and sequential use of different recombinant PIV-vectored vaccines in the same individual, as discussed above. In addition, we confirmed previous observations that PIV constructs can be stably propagated to high yields in vitro, and that no recombination restoring whole virus occurs after prolonged passaging in substrate cells (Mason et al., Virology 351:432-443, 2006; Shustov et al., J. Virol. 21:11737-11748, 2007).

These and other aspects of the invention are further described in the Examples, below.

### Example 1

### Pseudoinfectious Virus Platform Development Studies

### Attenuation in Suckling Mouse Neurovirulence (NV) Model

Materials used in the studies described below are described in Table 1 and the references cited therein. These include s-PIV-WN (based on wt WN virus strain NY99 sequences), s-PIV-JE, s-PIV-WN/JE (based on wt WN virus backbone and prM-E genes from wt JE virus Nakayama strain), s-PIV-YF/WN(YF 17D backbone and prM-E genes from WN virus), and s-PIV-YF (based on YF 17D sequences). Additional materials include d-PIV-YF (YF d-PIV, grown in regular BHK cells (Shustov et al., J. Virol. 21:11737-11748, 2007), and two-component d-PIV-WN (grown in regular Vero cells; Suzuki et al., J. Virol. 82:6942-6951, 2008).

Attenuation of these PIV prototypes was compared to LAVs YF 17D, a chimeric YF/JE virus, and a chimeric YF/WN virus in suckling mouse NV test (IC inoculation) using highly susceptible 5-day old ICR mice (the chimeric viruses include yellow fever capsid and non-structural sequences, and JE or WN prM-E sequences). None of the animals that received PIV constructs showed clinical signs or died, while mortality was observed in animals inoculated with LAVs (Table 2). The YF 17D virus is neurovirulent for mice of all ages, while the chimeric vaccines are not neurovirulent for adult mice, but can cause dose-dependent mortality in more sensitive suckling mice (Guirakhoo et al., Virology 257:363-372, 1999; Arroyo et al., J. Virol. 78:12497-12507, 2004). Accordingly, 90%-100% of suckling mice that received doses as low as 1 PFU of YF 17D died. YF/JE and YF/WN LAVs caused partial mortality at much higher doses (>2 log₁₀ PFU and 3 log₁₀ PFU, respectively), with longer average survival time (AST) of animals that died, as expected. Thus, PIV constructs are completely avirulent in this sensitive model (at least 20,000-200,000 times less neurovirulent than the licensed YF 17D vaccine).

The YF d-PIV and WN d-PIV caused no mortality or clinical signs. Thus, the two-component PIV variants that theoretically could spread within brain tissue from cells co-infected by both of their components did not cause disease. Moreover, we tried to detect the d-PIVs in the brains of additional animals in this experiment, sacrificed on day 6 post-inoculation by titration, and detected none (brain tissues from 10 and 11 mice that received 4 logjo FFU of YF d-PIV and WN d-PIV, respectively, were homogenized and used for titration). Thus, the d-PIVs did not cause spreading infection characteristic of whole virus. YF/JE LAV has been shown to replicate in the brain of adult ICR mice inoculated by the IC route with a peak titer of 6 log₁₀ PFU/g on day 6, albeit without clinical signs (Guirakhoo et al., Virology 257:363-372, 1999). Co-infection of cells with components of a d-PIV is clearly a less efficient process than infection with whole virus. The data show that d-PIV replication in vivo is quickly brought under control by innate immune responses (and adaptive responses in older animals).

### Immunogenicity/efficacy in Mice and Hamsters

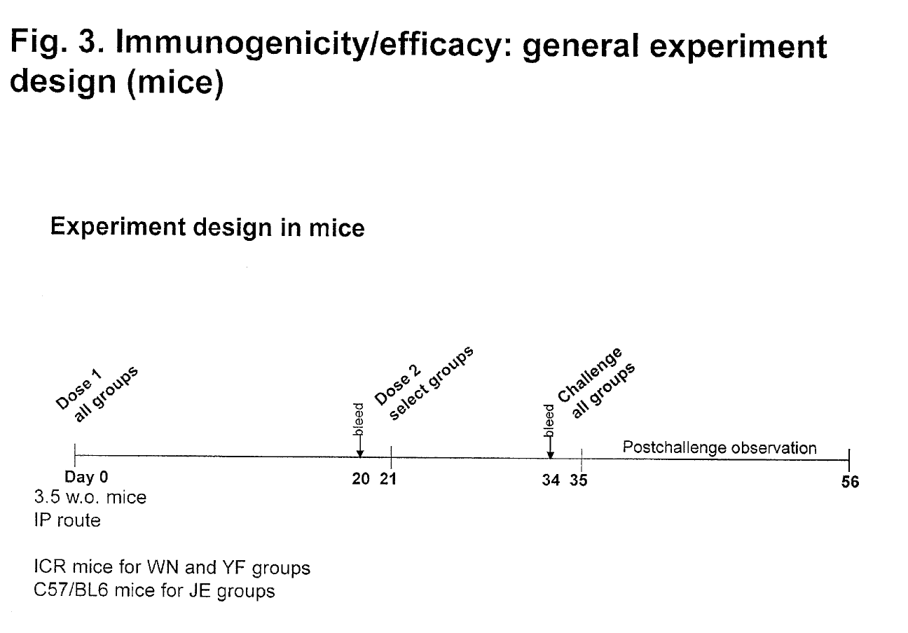

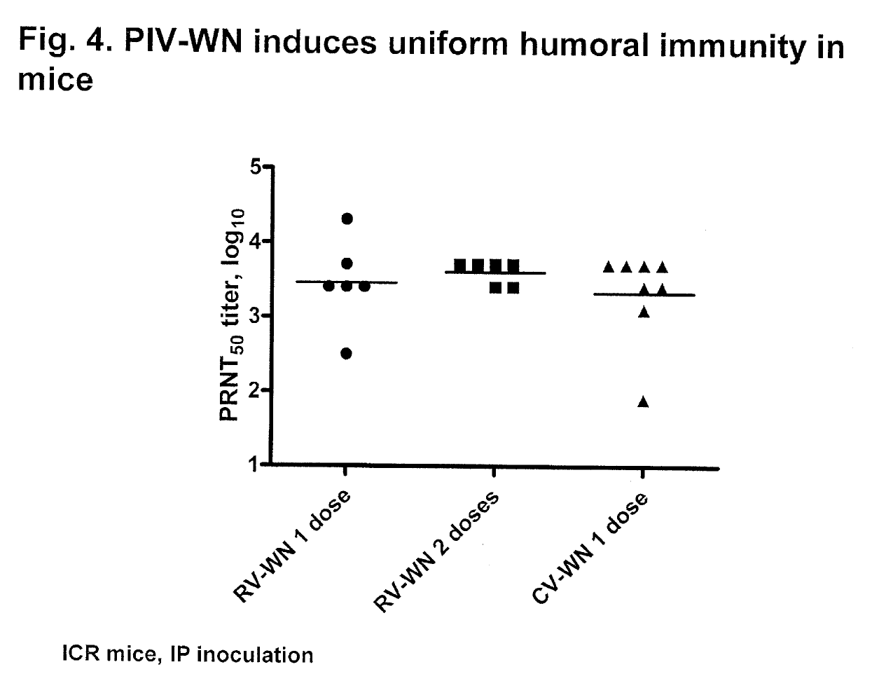

Immunogenicity/efficacy of the PIV prototypes described above was compared to that of chimeric LAV counterparts and YF 17D in mice and Syrian hamsters. The general experiment design is illustrated in FIG. 3 (mice, IP immunization). Experiments in hamsters were performed similarly (plus-minus a few days, SC or IP inoculation with doses indicated below). 3.5-week old ICR mice (for s-PIV-WN and -YF, YF/WN LAV, and YF 17D groups) or C57/BL6 mice (for s-PIV-JE and YF/JE LAV groups) were immunized IP with graded doses of PIV constructs (4-6 log₁₀ FFU/dose) or chimeric LAV and YF 17D LAV controls (4 log₁₀ PFU). Select PIV-WN, -JE and -YF groups were boosted on day 21 with 5 log₁₀ FFU of corresponding constructs (Table 3). Neutralizing antibody responses were determined in animal sera by standard PRNT₅₀ against YF/WN or /JE LAVs, or YF 17D viruses. PIV-WN induced very high WN-specific neutralizing antibody responses in all groups, with or without boost, as evidenced by PRNT₅₀ titers determined in pools of sera from immunized animals on days 20 and 34, which was comparable to that in the YF/WN LAV control group. Accordingly, animals immunized with both PIV-WN and YF/WN LAV were protected from lethal challenge on day 35 with wt WN virus (IP, 270 LD₅₀), but not mock-immunized animals (Table 3). When WN neutralizing antibodies were measured in sera from individual mice, high uniformity of immune responses was observed (FIG. 4). Thus, single-round PIV vaccines can be as immunogenic and efficacious as corresponding LAVs. PIV-JE was also highly immunogenic (black mice), while immunogenicity of PIV-YF was significantly lower compared to the YF 17D control (ICR mice). Yet, dose-dependent protection of PIV-YF immunized animals (but not mock-immunized animals) was observed following a severe lethal IC challenge with wt YF strain Asibi virus (500 LD₅₀) (Table 3), which is in agreement with the knowledge that neutralizing antibody titers as low 1:10 are protective against flavivirus infections.

The YF 17D control virus was highly immunogenic (e.g., PRNT₅₀ titer 1:1,280 on day 34), and thus it is able to infect cells and replicate efficiently in vivo, and its envelope is a strong immunogen. Therefore, it is unlikely that low immunogenicity of PIV-YE was due to its inability to infect cells or replicate efficiently in infected cells in vivo. We believe that the low immunogenicity of PIV-YF (e.g., compared to PIV-WN) was most likely due to a low-level production of YF-specific VLPs in PIV-YF infected cells (while VLP secretion is high in PIV-WN infected cells). As discussed above, we propose that immunogenicity of PIV-YF can be significantly increased, e.g., by appropriate modifications at the C/prM junction, e.g., by uncoupling the two protease cleavages that occur at this junction (viral protease and signalase cleavages), and/or by using a strong heterologous signal [e.g., rabies virus G protein signal, or eukaryotic tissue plasminogen activator (tPA) signal (Malin et al., Microbes and Infection, 2:1677-1685, 2000), etc.] in place of the YF signal for prM.

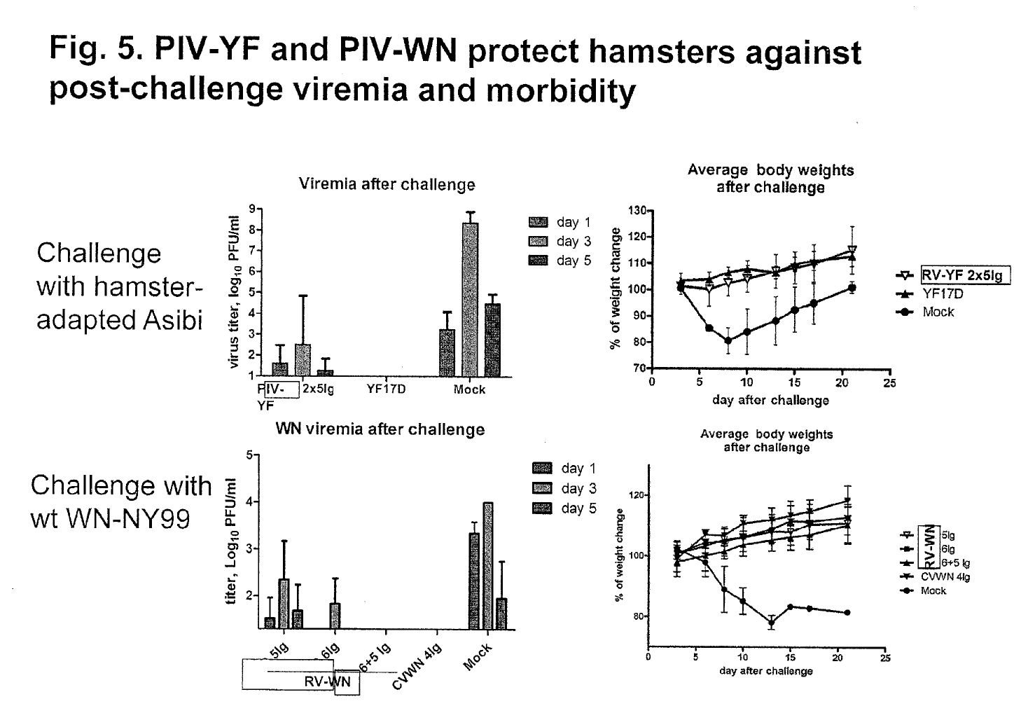

A similar experiment was performed in ˜4.5-week old Syrian hamsters, to compare immunogenicity of PIV constructs to LAV controls in this model. Animals were immunized SC with graded doses of the test articles (Table 4). PIV-WN was highly immunogenic, e.g., WN-specific PRNT₅₀ titers on day 38 (pre-challenge) were 1:320, 1:640, and 1:1280 in groups that received 5, 6, and 6 (prime)+5 (boost) log₁₀ FFU doses, respectively. This was somewhat lower compared to YF/WN LAV 4 log₁₀ PFU control (≧1:2560). PIV-JE and -YF induced detectable specific neutralizing antibody responses, albeit with lower titers compared to YF/JE LAV and YF 17D controls. All animals immunized with PIV-WN and YF/WN were solidly protected from lethal challenge with wt WN virus as evidenced by the absence of mortality and morbidity (e.g., loss of body weight after challenge), as well as absence or a significant reduction of postchallenge WN virus viremia. Mock-immunized animals were not protected (Table 4). PIV-JE and -WN protected animals from respective challenge in dose-dependent fashion. Protective efficacy in this experiment is additionally illustrated in FIG. 5. For example, high post-challenge YF virus (hamster adapted Asibi strain) viremia was observed in mock immunized animals, peaking on day 3 at a titer of >8 log₁₀ PFU/ml (upper left panel); all of the animals lost weight, and 1 out of 4 died (upper right panel). In contrast, viremia was significantly reduced or absent in hamsters immunized with PIV-YF (two doses; despite relatively low neutralizing titers) or YF 17D; none of these animals lost weight. Similarly, animals immunized with PIV-WN or YF/WN LAV were significantly or completely protected in terms of post-challenge WN virus viremia and body weigh loss/mortality, in contrast to mock controls (compare in bottom panels). Thus, high immunogenicity/efficacy of PIV was demonstrated in a second animal model.

In another hamster experiment, animals were immunized with PIV constructs by the IP route, with two doses. Table 5 compares neutralizing immune responses (specific for each vaccine) determined in pooled sera of hamsters in the above-described experiment (SC inoculation) to those after IP immunization, for PIV-WN, -YF/WN, -WN/JE, and -YF after the first dose (days 20-21) and second dose (days 34-38). A clear effect of the immunization route was observed both after the 1^{st} and 2^{nd} doses. For instance, for PIV-WN after 1^{st} dose, SC immunization resulted in WN-specific PRNT50 titer of 1:40, while IP inoculation resulted in much higher titer 1:320 (and after the 2^{nd} dose, titers were similar). A more pronounced effect was observed for other constructs after both the 1^{st} and 2^{nd} doses. Interestingly, PIV-YF/WN was very highly immunogenic by IP route (titer 1:320 after 1^{st} IP dose vs. 1:20 by SC, and 1:1,280 after 2^{nd} dose vs. 1:160 by SC). Similarly, immunogenicity of PIV-JE was significantly increased (e.g., JE-specific titer of 1:640 after two IP poses). Thus, better targeting of lymphoid cells, specifically antigen-presenting cells (which are more abundant in the abdomen as opposed to tissues under the skin), is an important consideration for use of PIV vaccines. In humans, efficient targeting of dendritic cells of the skin, increasing the magnitude of immune response, can be achieved by intradermal delivery, which we thus propose for a route for PIV immunization of humans.

In the above-described experiments, we also determined whether a neutralizing antibody response was induced against packaging envelopes (as opposed to response to VLPs encoded by PIV constructs and secreted by infected cells). No WN-specific neutralizing antibodies were detected by PRNT₅₀ in animals immunized with 5 log₁₀FFU of the second component of WN d-PIV, containing the ΔC-prM-E deletion and thus not encoding VLPs, but packaged into the WN envelope in BHK-CprME(WN) helper cells, and no YF-specific neutralizing activity was found in sera from animals immunized with 4 log₁₀ FFU of the second component of YF d-PIV packaged in YF envelope. No YF-specific neutralizing response was induced by two doses of PIV-YF/WN packaged into YF envelope, and similarly, no WN-specific response was induced by two doses of PIV-JE packaged into WN envelope. The absence of neutralizing response against packaging envelopes permits manufacturing different PIV vaccines in one (universal) manufacturing helper cell line, or immunization of one individual with different recombinant vaccines based on the same vector, according to the present invention.

### PIV Cocktails

Because PIVs undergo a single (optionally several, but limited) round(s) of replication in vivo, we considered that mixtures of different PIV vaccines can be administered without interference between individual constructs in the mixture (cocktail). To elucidate whether PIV vaccines can be used in cocktail formulations, immune responses in mice and hamsters to several PIV constructs given as mixtures were compared to the same constructs given individually. Similar results were obtained in both animal models. Results of mouse experiments are shown in Table 6. Similar anti-JE neutralizing antibody titers were observed in pools of sera from animals that were given one or two doses of either PIV-JE+PIV-WN mixture or PIV-JE alone (1:20 vs. 1:80 and 1:640 vs. 1:160, for one and two doses, respectively). Similarly, WN-specific titers against PIV-JE+PIV-WN mixture and PIV-WN alone were similar (1:320 vs. 1:640 and 1:5,120 vs. 1:5,120 for one and 2 doses, respectively). No or little cross-specific response was induced by either PIV-JE or -WN. The result was also confirmed by measuring PRNT₅₀ titers in sera from individual animals. Thus, it is clear that PIV vaccines can be efficiently administered as cocktails, inducing immunity against two or more flavivirus pathogens. In addition, as discussed above, various cocktails can be made between non-flavivirus PIV vaccines, or between any of flavivirus and non-flavivirus PIV vaccines.

### In Vitro Studies

Different PIV prototypes were serially passaged up to 10 times in helper BHK cells, for s-PIVs, or in regular Vero cells, for d-PIVs. Samples harvested after each passage were titrated in Vero cells by immunostaining. Constructs grew to high titers, and no recombination restoring whole virus was observed. For instance, PIV-WN consistently grew to titers 7-8 log₁₀ FFU/ml in BHK-CprME(WN) helper cells (containing a VEE replicon expressing the WN virus C-prM-E proteins), and WN d-PIV grew to titers exceeding 8 log₁₀ FFU/ml in Vero cells, without recombination.

### Example 2

### PIV-TBE

PIV-TBE vaccine candidates can be assembled based entirely on sequences from wt TBE virus or the closely serologically related Langat (LGT) virus (naturally attenuated virus, e.g., wt strain TP-21 or its empirically attenuated variant, strain E5), or based on chimeric sequences containing the backbone (capsid and non-structural sequences) from YF 17D or other flaviviruses, such as WN virus, and the prM-E envelope protein genes from TBE, LGT, or other serologically related flaviviruses from the TBE serocomplex. YF/TBE LAV candidates are constructed based on the backbone from YF 17D and the prM-E genes from TBE or related viruses (e.g., the E5 strain of LGT), similar to other chimeric LAV vaccines.

Construction of PIV-TBE and YF/TBE LAV vaccine prototypes was performed by cloning of appropriate genetic elements into plasmids for PIV-WN (Mason et al., Virology 351:432-443, 2006; Suzuki et al., J. Virol. 82:6942-6951, 2008), or plasmids for chimeric LAVs (e.g., pBSA-AR1, a single-plasmid version of infectious clone of YF/JE LAV; WO 2008/036146), respectively, using standard methods in the art of reverse genetics. The prM-E sequences of TBE virus strain Hypr (GenBank accession number U39292) and LGT strain E5 (GenBank accession number AF253420) were first computer codon-optimized to conform to the preferential codon usage in the human genome, and to eliminate nucleotide sequence repeats longer than 8 nt to ensure high genetic stability of inserts (if determined to be necessary, further shortening of nt sequence repeats can be performed). The genes were chemically synthesized and cloned into plasmids for PIV-WN and YF/JE LAV, in place of corresponding prM-E genes. Resulting plasmids were in vitro transcribed and appropriate cells (Vero for chimeric viruses, and helper BHK cells for PIV) were transfected with RNA transcripts to generate virus/PIV samples.

### YF/TBE LAV Constructs

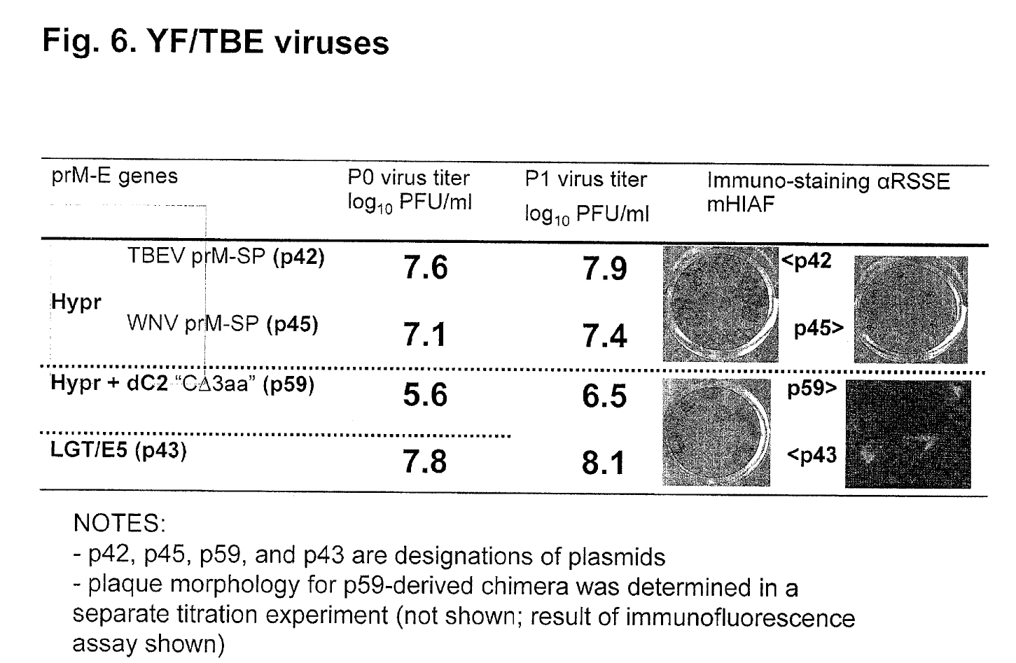

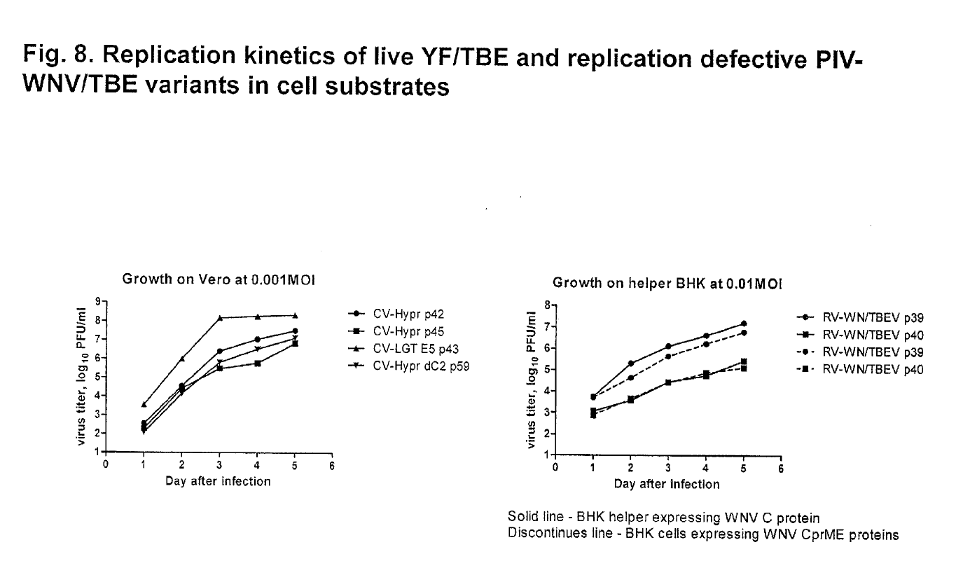

In YF/TBE constructs containing either the TBE Hypr (plasmids p42, p45, and p59) or LGT E5 (plasmid P43) prM-E genes, two different types of the C/prM junction were first examined (see in FIG. 6; C/prM junctions only are shown in Sequence Appendix 1, and complete 5′-terminal sequences covering the 5′UTR-C-prM-E-beginning of NS1 region are shown in Sequence Appendix 2). The p42-derived YF17D/Hypr chimera contained a hybrid YF17D/Hypr signal peptide for the prM protein, while the p45-derived YF17D/Hypr chimera contained a hybrid YF17D/WN signal peptide for prM (Sequence Appendix 1). The former chimeric virus produced very high titers at both P0 (immediately after transfection) and P1 (the next passage in Vero cells), up to 7.9 log₁₀PFU/ml, which were 0.5 log₁₀ times higher, compared to the latter virus; in addition it formed significantly larger plaques in Vero cells (FIG. 6). Thus, use of TBE-specific residues in the signal peptide for prM conferred a significant growth advantage over the signal containing WN-specific residues. The p43-derived YF17D/LGT chimera had the same prM signal as the p42-derived virus; it also produced very high titers at P0 and P1 passages (up to 8.1 log₁₀ PFU/ml) and formed large plaques. A derivative of the p42-derived virus was also produced from plasmid p59, which contained a strong attenuating mutation characterized previously in the context of a YF/WN LAV vaccine virus, specifically, a 3-a.a. deletion in the YF17D-specific C protein (PSR, residues 40-42 in the beginning of α-Helix I; WO 2006/116182). As expected, the p59 virus grew to lower titers (5.6 and 6.5 log₁₀ PFU/ml at P0 and P1, respectively), and formed small plaques (determined in a separate titration experiment and thus not shown in FIG. 6), compared to the parent p42-derived chimera. These initial observations of growth properties of YF/TBE LAV prototypes, and correlation of replication in vitro with plaque morphologies, have been confirmed in growth curve experiments (FIG. 8).

### PIV-TBE Constructs

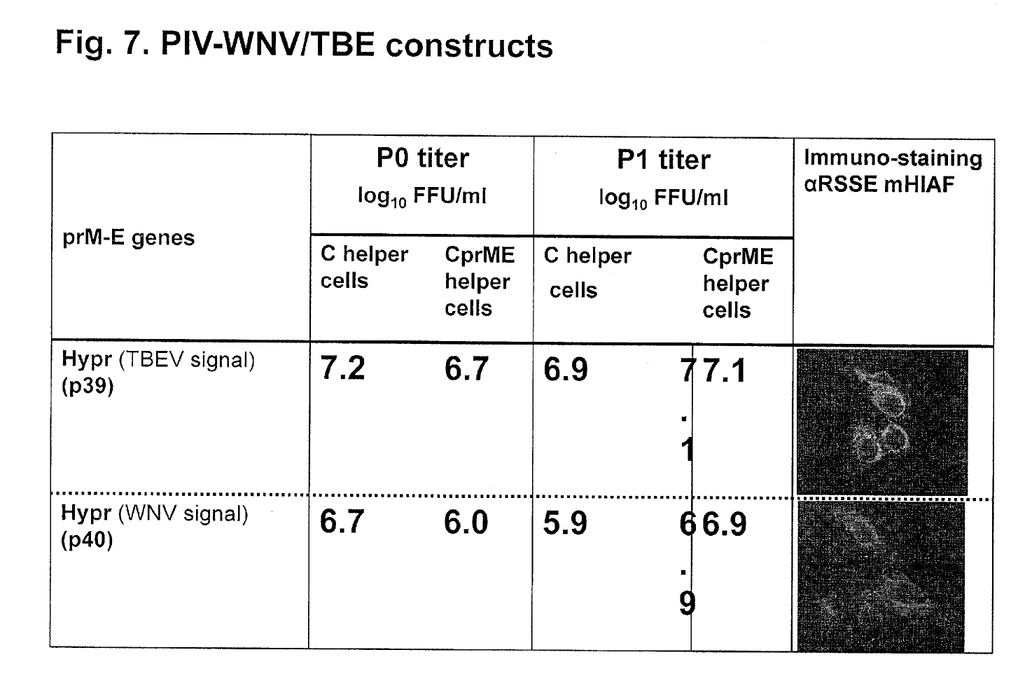

PIV-WN/TBE variants were constructed, and packaged PIV samples were derived from plasmids p39 and p40 (FIG. 7; Sequence Appendix 1 for C/prM junction sequences, and Sequence Appendix 3 for complete 5′UTR-ΔC-prM-E-beginning of NS1 sequences). These contained complete Hypr or WN prM signals, respectively. Both PIVs were successfully recovered and propagated in BHK-CprME(WN) or BHK-C(WN) helper cells (Mason et al., Virology 351:432-443, 2006; Widman et al., Vaccine 26:2762-2771, 2008). The P0 and P1 sample titers of the p39 variant were 0.2-1.0 log₁₀ times, higher than p40 variant. In addition, Vero cells infected with p39 variant were stained brighter in immunofluorescence assay using a polyclonal TBE-specific antibody, compared to p40, indicative of more efficient replication (FIG. 7). The higher rate of replication of the p39 candidate than p40 candidate was confirmed in a growth curve experiment (FIG. 8). In the latter experiment, both candidates appeared to grow better in the BHK-C(WN) helper cells compared to BHK-CprME(WN), with the p39 variant reaching titer of ˜7 log₁₀ PFU/ml on day 5 (note that peak titers have not been reached). The discovery of the effect of prM signal on replication rates of both PIV and chimeric LAV vaccine candidates, and head-to-head comparison of different signals to generate the most efficiently replicating and immunogenic (see above) construct, are a distinguishing feature of our approach. As discussed above, the invention also includes the use of other flavivirus signals, including with appropriate mutations, the uncoupling the viral protease and signalase cleavages at the C/prM junction, e.g., by mutating or deleting the viral protease cleavage site at the C-terminus of C preceding the prM signal, the use of strong non-flavivirus signals (e.g., tPA signal, etc.) in place of prM signal, as well as optimization of sequences downstream from the signalase cleavage site.

Other PIV-TBE variants based entirely on wt TBE (Hypr strain) and LGT virus (TP21 wild type strain or attenuated E5 strain), and chimeric YF 17D backbone/prM-E (TBE or LGT) sequences are also included in the invention. Helper cells providing appropriate C, C-prM-E, etc., proteins (e.g., TBE-specific) for trans-complementation can be constructed by means of stable DNA transfection or through the use of an appropriate vector, e.g., an alphavirus replicon, such as based on VEE strain TC-83, with antibiotic selection of replicon-containing cells. Vero and BHK21 cells can be used in practice of the invention. The former are an approved substrate for human vaccine manufacture; any other cell line acceptable for human and/or veterinary vaccine manufacturing can be also used. In addition to s-PIV constructs, d-PIV constructs can also be assembled. To additionally ascertain safety for vaccinees and the environment, appropriate modifications can be employed, including the use of degenerate codons and complementary mutations in the 5′ and 3′ CS elements, to minimize chances of recombination that theoretically could result in viable virus. Following construction, all vaccine candidates can be evaluated in vitro for manufacturability/stability, and in vivo for attenuation and immunogenicity/efficacy, in available pre-clinical animal models, such as those used in development and quality control of TBE and YF vaccines.

### Neurovirulence and Neuroinvasiveness in Mice of PIV-TBE and YF/TBE LAV Constructs

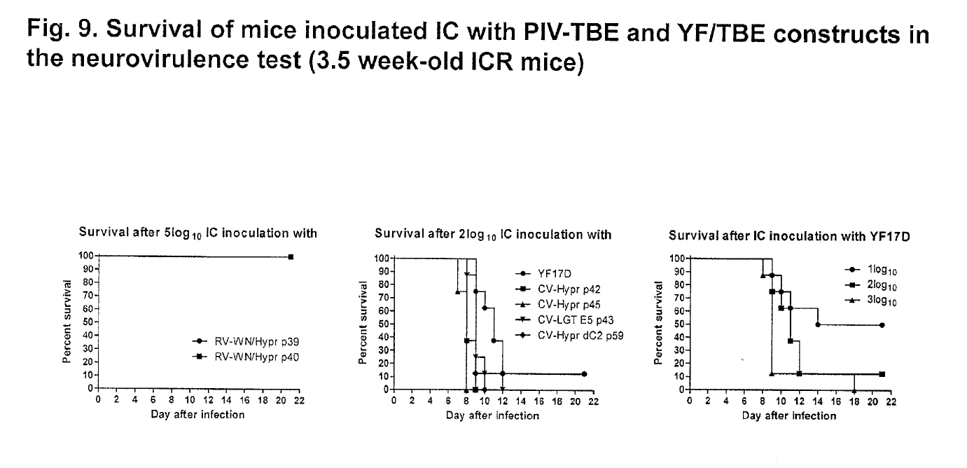

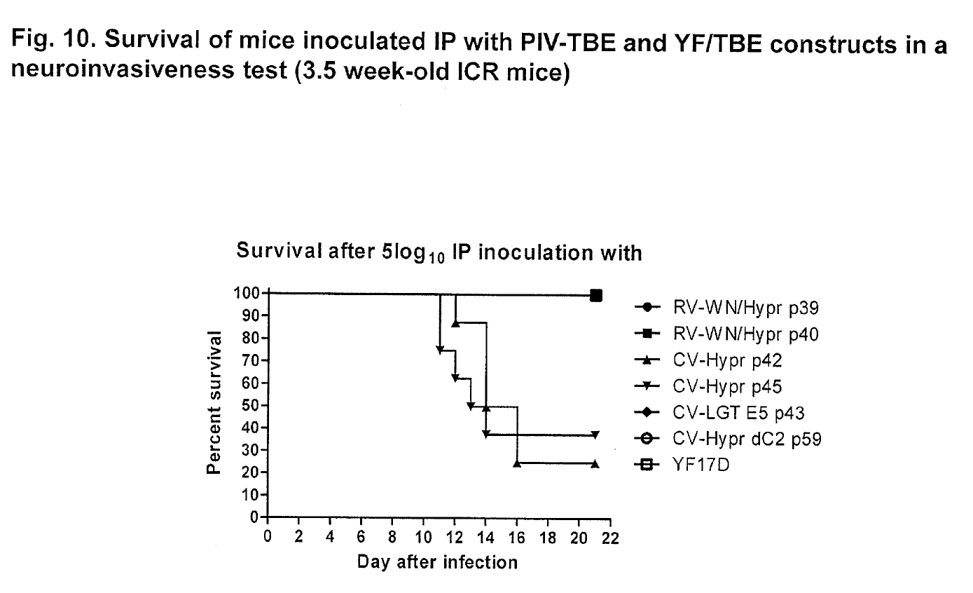

Young adult ICR mice (˜3.5 week-old), were inoculated with graded doses of PIV-TBE and YF/TBE LAV candidates by the IC route to measure neurovirulence, or IP route to measure neuroinvasiveness (and later immunogenicity/efficacy). Animals that received 5 log₁₀ FFU of PIV-Hypr (p39 and p40) variants by both routes survived and showed no signs of sickness, similar to mock-inoculated animals (Table 7), and thus PIV-TBE vaccines are completely avirulent. Mice inoculated IC with YF 17D control (1-3 log₁₀ PFU) showed dose-dependent mortality, while all animals inoculated IP (5 log₁₀ PFU) survived, in accord with the knowledge that YF 17D virus is not neuroinvasive. All animals that received graded IC doses (2-4 log₁₀ PFU) of YF/TBE LAV prototypes p42, p45, p43, and p59 died (moribund animals were humanely euthanized). These variants appear to be less attenuated than YF 17D, e.g., as evidenced by complete mortality and shorter AST at the 2 log₁₀ PFU dose, the lowest dose tested for YF/TBE LAV candidates. The non-neurovirulent phenotype of PIV-TBE, virulent phenotype of YF/TBE LAV and intermediate-virulence phenotype of YF 17D are also illustrated in FIG. 9, showing survival curves of mice after IC inoculation. It should be noted that the p43 (LGT prM-E genes) and p59 (the dC2 deletion variant of YF/Hypr LAV) were less neurovirulent than p42 and p45 YF/Hypr LAV constructs as evidenced by larger AST values for corresponding doses (Table 7). In addition, p43 and p59 candidates were non-neuroinvasive, while p42 and p45 caused partial mortality after IP inoculation (5 log₁₀ PFU/dose) (Table 7; FIG. 10). It should be noted however that all the YF/TBE LAV constructs were significantly attenuated as compared to wt TBE viruses, e.g., compared to wt TBE Hypr virus, which is uniformly highly virulent for mice, both at very low IC (LD₅₀˜0.1 PFU) and IP (LD₅₀≦10 PFU) doses (Wallner et al., J. Gen. Virol. 77:1035-1042, 1996; Mandl et al., J. Virol. 72:2132-2140, 1998; Mandl et al., J. Gen. Virol. 78:1049-1057, 1997

### Immunogenicity/efficacy of PIV-TBE and YF/TBE LAV Constructs in Mice

TBE-specific neutralizing antibody responses in mice immunized IP with one or two doses of the PIV-TBE or YF/TBE LAV variants described above, or a human formalin-inactivated TBE vaccine control (1:30 of human dose) are being measured. Animals have been challenged with a high IP dose (500 PFU) of wt Hypr TBE virus; morbidity (e.g., weight loss), and mortality after challenge are monitored.

### Immunogenicity/efficacy of PIV-TBE and YF/TBE LAV Constructs in Mice

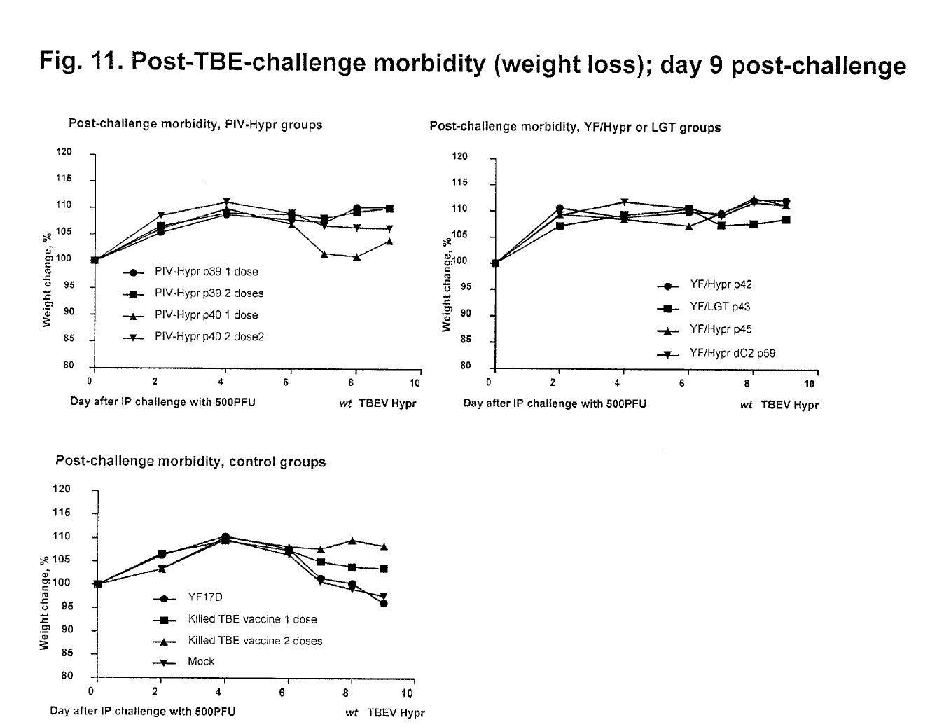

TBE-specific neutralizing antibody responses in mice immunized IP with one or two doses of the PIV-TBE or YF/TBE LAV variants described above (from experiment in Table 7), or a human formalin-inactivated TBE vaccine control (1:20 of human dose; one or two doses), or YF 17D and mock controls, were measured on day 20 by PRNT₅₀ against wt TBE Hypr virus (Table 8; second dose of indicated test articles was given on day 14). [Titers were determined in individual sera, or pooled sera from two animals in most cases, or pooled sera from 4 animals for the YF 17D and Mock negative controls]. Titers in individual test samples as well as GMTs for each group are provided in Table 8. Titers in test samples were similar within each group, e.g., in groups immunized with PIVs, indicating high uniformity of immune response in animals. As expected, no TBE-specific neutralizing antibodies were detected in negative control groups (YF 17D and Mock; GMTs <1:10); accordingly, animals in these groups were not protected from challenge on day 21 post-immunization with a high IP dose (500 PFU) of wt Hypr TBE virus. Mortalities from partial observation (on day 9 post-challenge; observation being continued) are provided in Table 8, and dynamics of average post-challenge body weights indicative of morbidity are shown in FIG. 11. Neutralizing antibodies were detected in killed vaccine controls, which were particularly high after two doses (GMT 1:1,496); animals in the 2-dose group were completely protected in that there was no mortality or body weight loss (but not animals in the 1-dose group). Animals that received both one and two doses of PIV-Hypr p39 had very high antibody titers (GMTs 1:665 and 1:10,584) and were solidly protected, demonstrating that robust protective immunity can be induced by s-PIV-TBE defective vaccine. The two animals that survived immunization with YF/Hypr p42 chimera (see in Table 7) also had high antibody titers (GMT 1:6,085) and were protected (Table 8; FIG. 11). Interestingly, PIV-Hypr p40 and YF/Hypr p45 were poorly immunogenic (GMTs 1:15 and 1:153 for one and two doses, and 1:68, respectively). As discussed above, these contained WN-specific sequences in the signal for prM, while the highly immunogenic PIV-Hypr p39 and YF/Hypr p42 constructs contained TBE-specific signal sequences. In agreement with discussion above, this result demonstrates the importance of choosing the right prM signal, e.g., the TBE-specific signal, to achieve high-level replication/VLP secretion, which in this experiment in vivo resulted in drastically different immune responses. Immunogenicity of YF/LGT p43 and YF/Hypr dC2 p59 chimeras was relatively low which could be expected, because of the use of a heterologous envelope (LGT, different from challenge TBE virus) and high attenuating effect of the dC2 deletion, respectively.

### Example 3

### Foreign Gene Expression

In the examples of recombinant PIV constructs described below, genes of interest were codon optimized (e.g., for efficient expression in a target vaccination host) and to eliminate long nt sequence repeats to increase insert stability (≧8 nt long; additional shortening of repeats can be performed if necessary), and then chemically synthesized. The genes were cloned into PIV-WN vector plasmids using standard methods of molecular biology well known in the art, and packaged PIVs were recovered following in vitro transcription and transfection of appropriate helper (for s-PIVs) or regular (for d-PIVs) cells.

Expression of Rabies Virus G Protein in WN s-PIV and d-PIV

Rabies virus, Rhabdoviridae family, is a significant human and veterinary pathogen. Despite the availability of several (killed) vaccines, improved vaccines are still needed for both veterinary and human use (e.g. as an inexpensive pre-exposure prophylactic vaccines). Rabies virus glycoprotein G mediates entry of the virus into cells and is the main immunogen. It has been expressed in other vectors with the purpose of developing veterinary vaccines (e.g., Pastoret and Brochier, Epidemio. Infect. 116:235-240, 1996; Li et al., Virology 356:147-154, 2006).

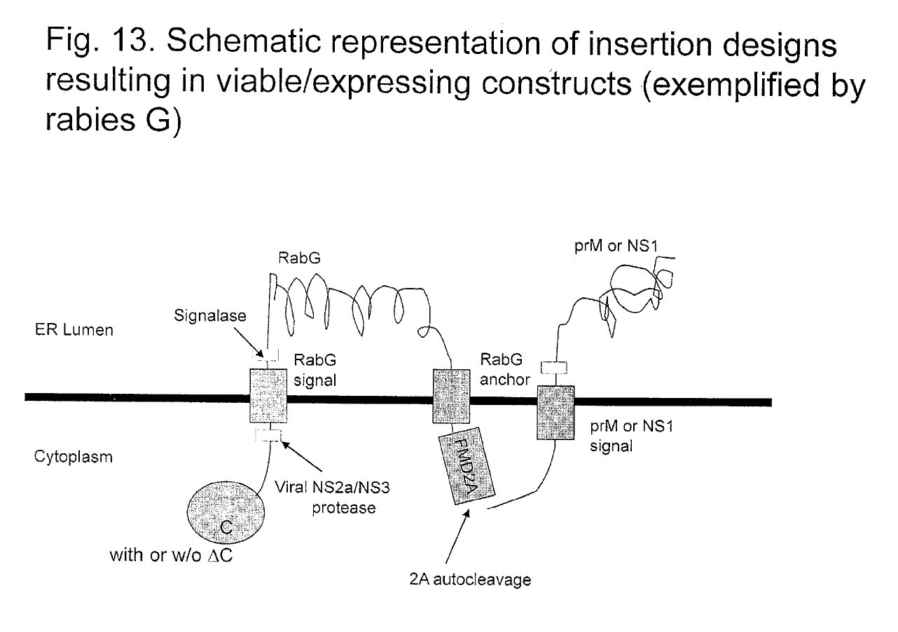

Full length rabies virus G protein (original Pasteur virus isolate, GenBank accession number NC_{—}001542) was codon-optimized, chemically synthesized, and inserted adjacent to the ΔC, ΔprM-E and ΔC-prM-E deletions in PIV-WN vectors (FIG. 12). The sequences of constructs are provided in Sequence Appendix 4. General designs of the constructs are illustrated in FIG. 13. The entire G protein containing its own signal peptide was inserted in-frame downstream from the WN C protein either with the ΔC deletion (ΔC and ΔC-prM-E constricts) or without (ΔprM-E) and a few residues from the prM signal. Foot and mouth disease virus (FMDV) 2A autoprotease was placed downstream from the transmembrane C-terminal anchor of G to provide cleavage of C-terminus of G from the viral polyprotein during translation. The FMDV 2A element is followed by WN-specific signal for prM and prM-E-NS1-5 genes in the ΔC construct, or signal for NS1 and NS1-5 genes in ΔprM-E and ΔC-prM-E constructs.

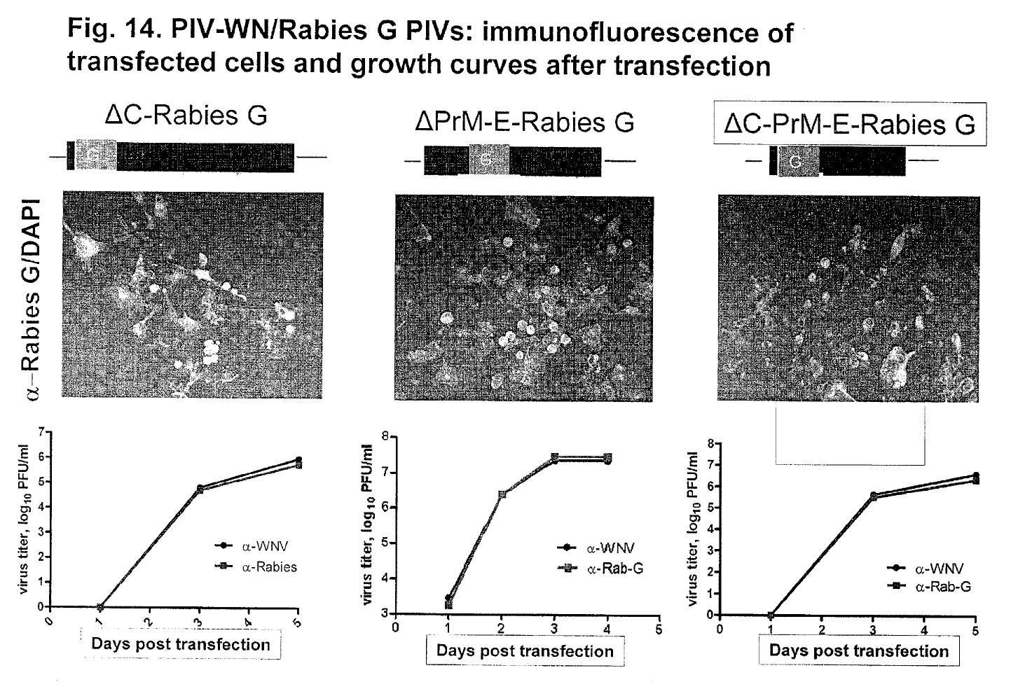

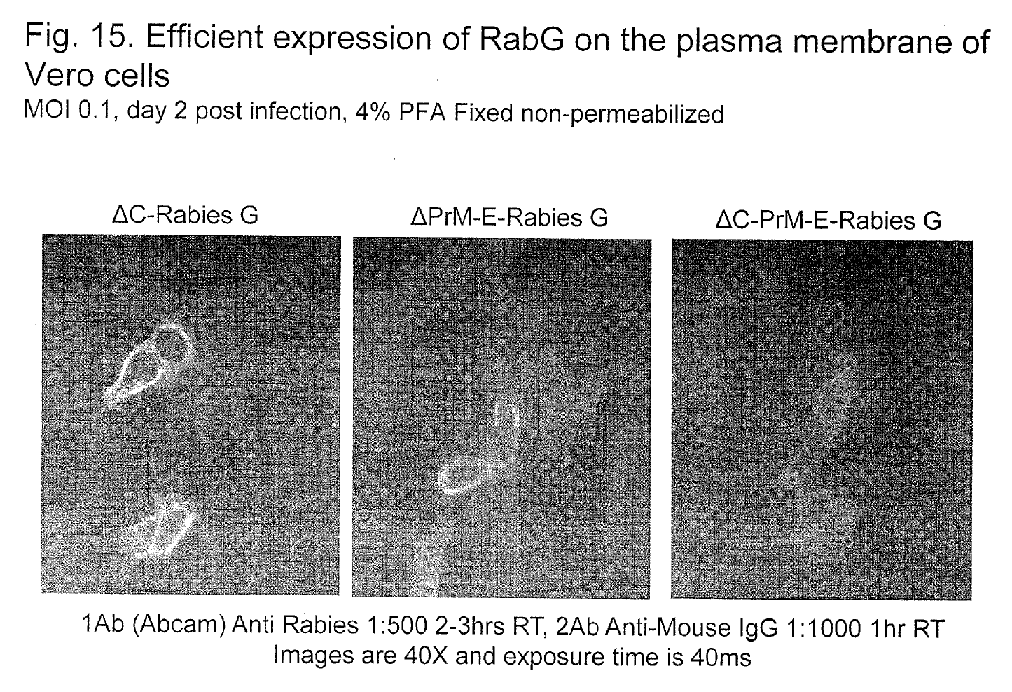

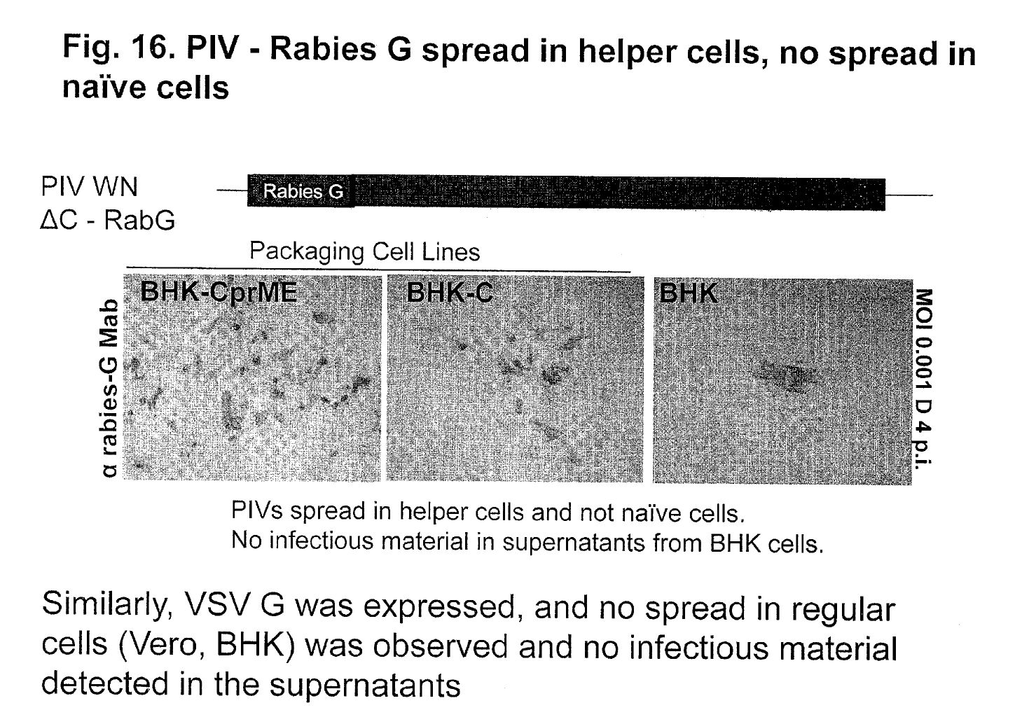

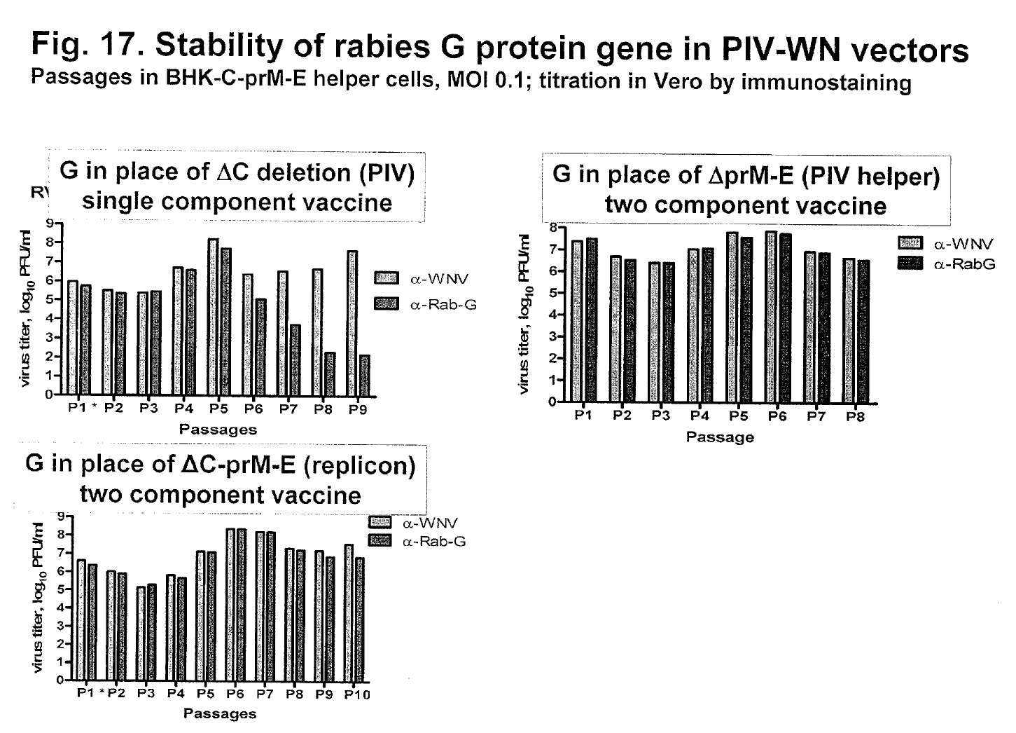

Packaged WN(ΔC)-rabiesG, WN(ΔprME)-rabiesG, and WN(ΔCprME)-rabiesG PIVs were produced by transfection of helper BHK cells complementing the PIV vector deletion [containing a Venezuelan equine encephalitis virus (strain TC-83) replicon expressing WN virus structural proteins for trans-complementation]. Efficient replication and expression of rabies G protein was demonstrated for the three constructs by transfection/infection of BHK-C(WN) and/or BHK-C-prM-E(WN) helper cells, as well as regular BHK cells, by immunostaining and immunofluorescence assay (IFA) using anti-Rabies G monoclonal antibody (RabG-Mab) (FIG. 14). Titers were determined in Vero cells by immunostaining with the Mab or an anti-WN virus polyclonal antibody. Growth curves of the constructs in BHK-CprME(WN) cells after transfection with in vitro RNA transcripts are shown in FIG. 14, bottom panels. The PIVs grew efficiently to titers ˜6 to >7 log₁₀ FFU/ml. Importantly, nearly identical titers were detected by both RabG-Mab and WN-antibody staining, which was the first evidence of genetic stability of the insert. In PIV-infected Vero cells, which were fixed but not permeabilized, strong membrane staining was observed by RabG-Mab staining, demonstrating that the product was efficiently delivered to the cell surface (FIG. 15). The latter is known to be the main prerequisite for high immunogenicity of expressed G. Individual packaged PIVs can spread following infection of helper BHK cells, but cannot spread in regular cells as illustrated for WN(ΔC)-rabiesG PIV in FIG. 16. The fact that there is no spread in naïve BHK cells demonstrates that the recombinant RNA genomes cannot be non-specifically packaged into membrane vesicles containing the G protein, if produced by PIV infected cells. An identical result was obtained with the G protein of another rhabdovirus, Vesicular stomatitis virus (VSV), contrary to previous observations of non-specific packaging of Semliki Forest virus (SFV) replicon expressing VSV G protein (Rolls et al., Cell 79:497-506, 1994). The latter is a desired safety feature. [Alternatively, some non-specific packaging could result in a limited spread of PIV in vivo, potentially enhancing anti-rabies immune response. The latter could be also a beneficial feature, given that such PIV is demonstrated to be safe]. The stability of the rabies G insert in the three PIVs was demonstrated by serial passages in helper BHK-CprME(WN) cells at high or low MOI (0.1 or 0.001 FFU/cell). At each passage, cell supernatants were harvested and titrated in regular cells (e.g., Vero cells) using immunostaining with an anti-WN polyclonal antibody to determine total PIV titer, or anti-rabies G monoclonal antibody to determine titer of particles containing the G gene (illustrated for MOI 0.1 in FIG. 17; similar results were obtained at MOI 0.001). The WN(ΔC)-rabiesG PIV was stable for 5 passages, while the titer of insert-containing PIV started declining at passage 6, indicative of insert instability. This could be expected, because in this construct, large G gene insert (˜1500 nt) is combined with a small AC deletion (˜200 nt), significantly increasing the overall size of the recombinant RNA genome. In contrast, in WN(ΔprME)-rabiesG, and WN(ΔCprME)-rabiesG PIVs, the insert is combined with a much larger deletion (˜2000 nt). Therefore, these constructs stably maintained the insert for all 10 passages examined (FIG. 17). Further, it can be seen in FIG. 17 that at some passages, titers as high as 8 log₁₀ FFU/ml, or higher, were attained for all three PIVs, additionally demonstrating that PIVs can be easily propagated to high yields.

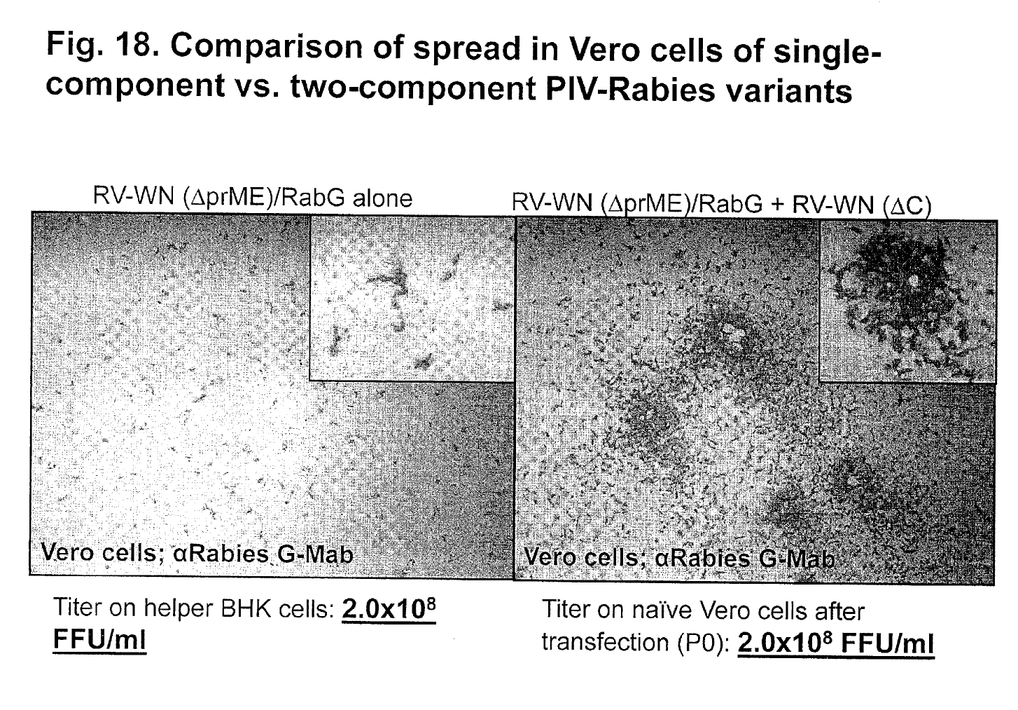

Following inoculation in vivo individually, the WN(ΔC)-rabiesG s-PIV is expected to induce strong neutralizing antibody immune responses against both rabies and WN viruses, as well as T-cell responses. The WN(ΔprME)-rabiesG and WN(ΔCprME)-rabiesG PIVs will induce humoral immune response only against rabies because they do not encode the WN prM-E genes. WN(ΔC)-rabiesG s-PIV construct can be also co-inoculated with WN(ΔprME)-rabiesG construct in a d-PIV formulation (see in FIG. 12), increasing the dose of expressed G protein, and with enhanced immunity against both pathogens due to limited spread. As an example of spread, titration results in Vero cells of a s-PIV sample, WN(ΔprME)-rabiesG, and a d-PIV sample, WN(ΔprME)-rabiesG+WN(ΔC) PIV (the latter did not encode rabies G protein), are shown in FIG. 18. Infection of naïve Vero cells with s-PIV gave only individual cells stainable with RabG-Mab (or small clusters formed due to division of cells). In contrast, large foci were observed following infection with the d-PIV sample (FIG. 18, right panel) that were products of coinfection with the two PIV types.

The WN(ΔCprME)-rabiesG construct can be also used in a d-PIV formulation, if it is co-inoculated with a helper genome providing C-prM-E in trans (see in FIG. 12). For example it can be a WN virus genome containing a deletion of one of the NS proteins, e.g., NS1, NS3, or NS5, which are known to be trans-complementable (Khromykh et al., J. Virol. 73:10272-10280, 1999; Khromykh et al., J. Virol. 74:3253-3263, 2000). We have constructed a WN-ΔNS1 genome (sequence provided in Sequence Appendix 4) and obtained evidence of co-infection with WN(ΔprME)-rabiesG or WN(ΔCprME)-rabiesG constructs, and spread in vitro, by immunostaining. In the case of such d-PIVs, rabies G protein can be also inserted and expressed in helper genome, e.g., WN-ΔNS1 genome, to increase the amount of expressed rabies G protein resulting in an increased anti-rabies immune response. As with any dPIV versions, one immunogen can be from one pathogen (e.g., rabies G) and the other from a second pathogen, resulting in three antigenic specificities of vaccine. As discussed above, ΔNS1 deletions can be replaced with or used in combination with ΔNS3 and/or ΔNS5 deletions/mutations, in other examples.

Expression of RSV F Protein in WN s-PIV and d-PIV

Respiratory syncytial virus (RSV), member of Paramyxoviridae family, is the leading cause of severe respiratory tract disease in young children worldwide (Collins and Crowe, Respiratory Syncytial Virus and Metapneumovirus, In: Knipe et al. Eds., Fields Virology, 5^{th} ed., Philadelphia: Wolters Kluwer/Lippincott Williams and Wilkins, 2007:1601-1646). Fusion protein F of the virus is a lead viral antigen for developing a safe and effective vaccine. To avoid post-vaccination exacerbation of RSV infection observed previously with a formalin-inactivated vaccine candidate, a balanced Th1/Th2 response to F is required which can be achieved by better TLR stimulation, a prerequisite for induction of high-affinity antibodies (Delgado et al., Nat. Med. 15:34-41, 2009), which should be achievable through delivering F in a robust virus-based vector. We have previously demonstrated the capacity of yellow fever virus-based chimeric LAV vectors to induce a strong, balanced Th1/Th2 response in vivo against an influenza antigen (WO 2008/036146). In the present invention, both yellow fever virus-based chimeric LAVs and PIV vectors are used for delivering RSV F to induce optimal immune response profile. Other LAVs and PIV vectors described herein can also be used for this purpose.

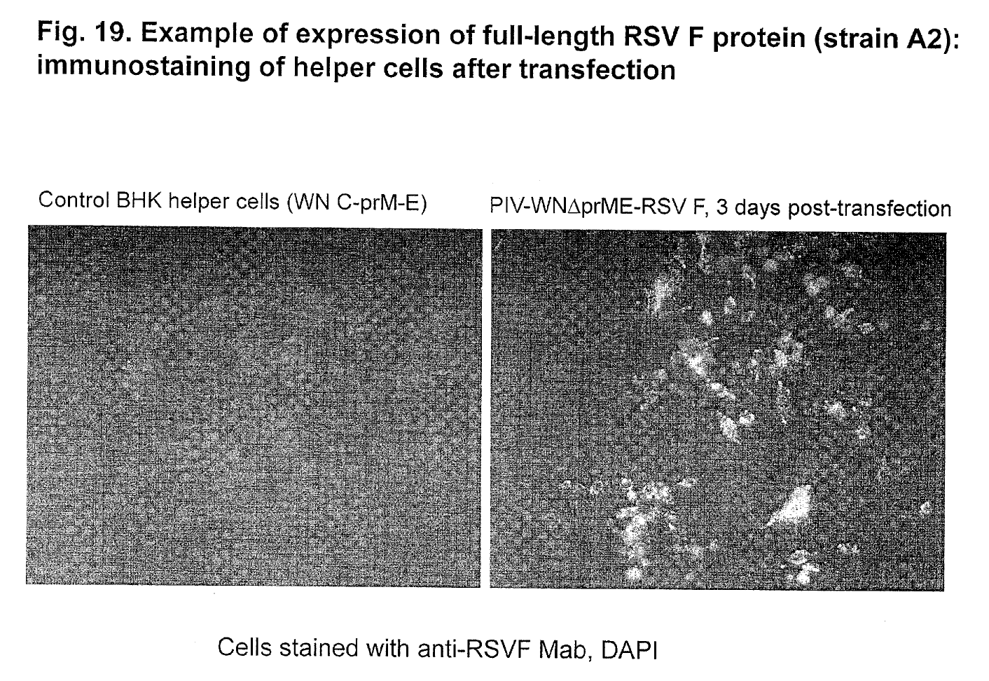

Full-length RSV F protein of A2 strain of the virus (GenBank accession number P03420) was codon optimized as described above, synthesized, and cloned into plasmids for PIV-WN s-PIV and d-PIV, using the insertion schemes shown in FIGS. 12 and 13 for rabies G protein, by applying standard methods of molecular biology. Exact sequences of the insertions and surrounding genetic elements are provided in Sequence Appendix 5. In vitro RNA transcripts of resulting WN(ΔC)-RSV F, WN(ΔprME)-RSV F, and WN(ΔCprME)-RSV F PIV constructs were used to transfect helper BHK-CprME(WN) cells. Efficient replication and expression of RSV F protein was first demonstrated by immunostaining of transfected cells with an anti-RSV F Mab, as illustrated for the WN(ΔprME)-RSV F construct in FIG. 19. The presence of packaged PIVs in the supernatants from transfected cells (titer as high as 7 log10 FFU/ml) was determined by titration in Vero cells with immunostaining. Additionally, similar constructs can be used that contain a modified F protein gene. Specifically, the N-terminal native signal peptide of F is replaced in modified F protein with the one from rabies virus G protein. The modification is intended to elucidate whether the use of a heterologous signal can increase the rate of F protein synthesis and/or replication of PIVs.

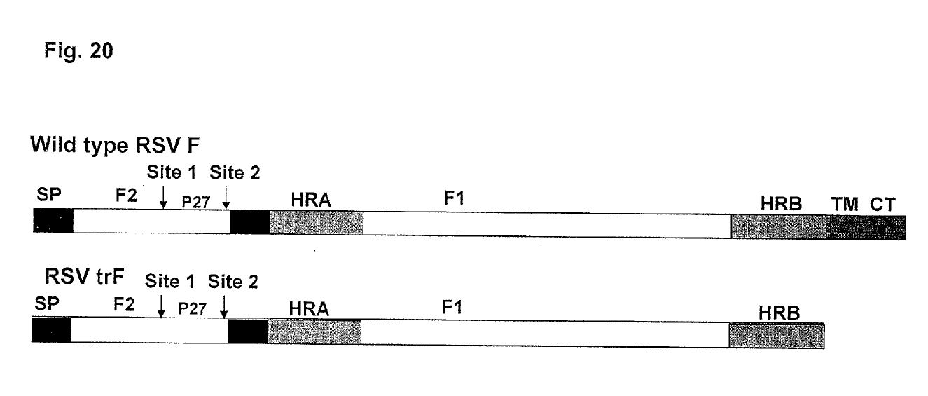

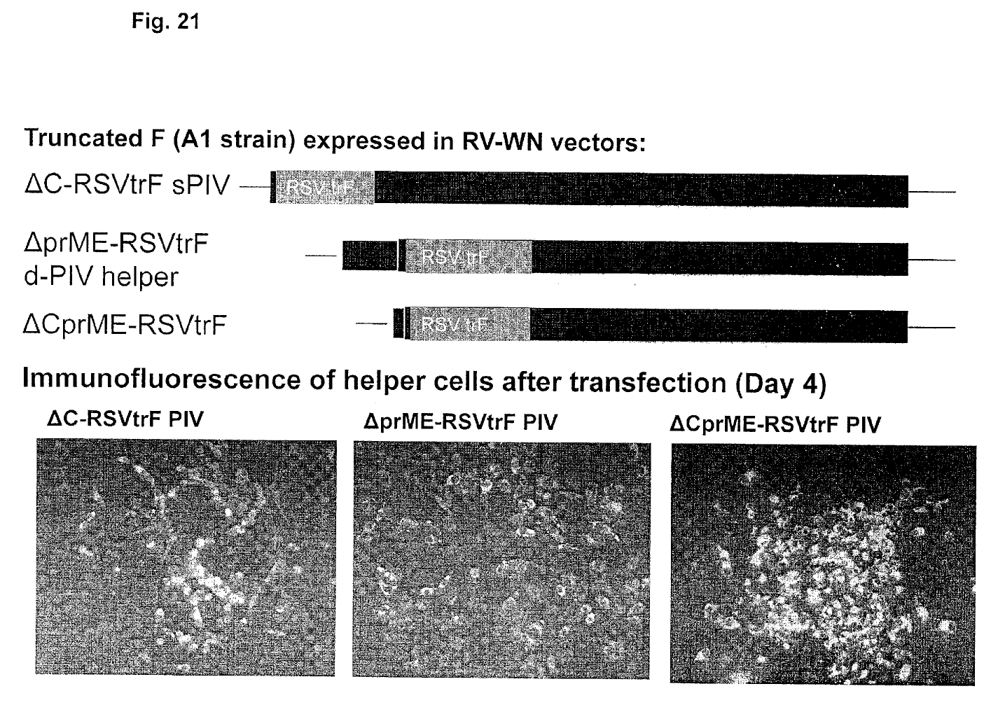

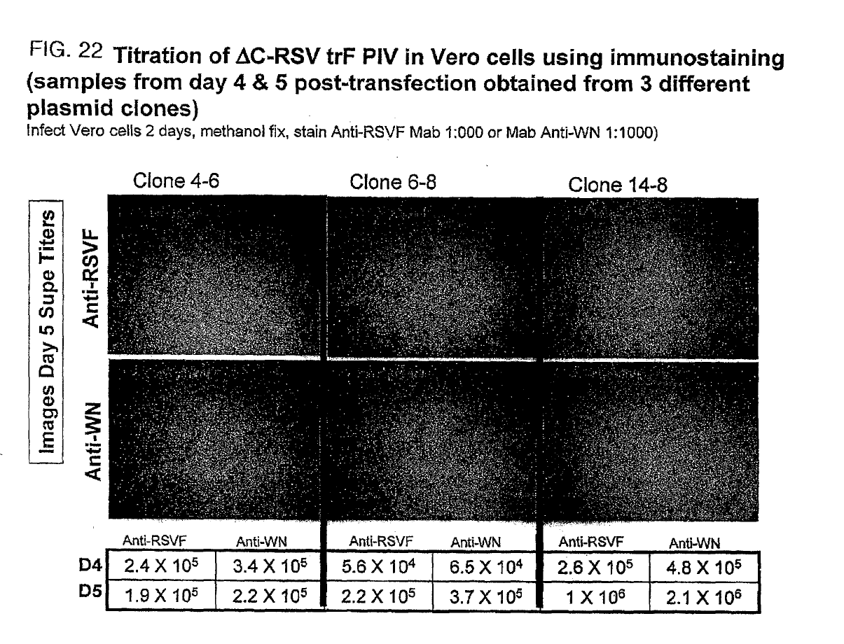

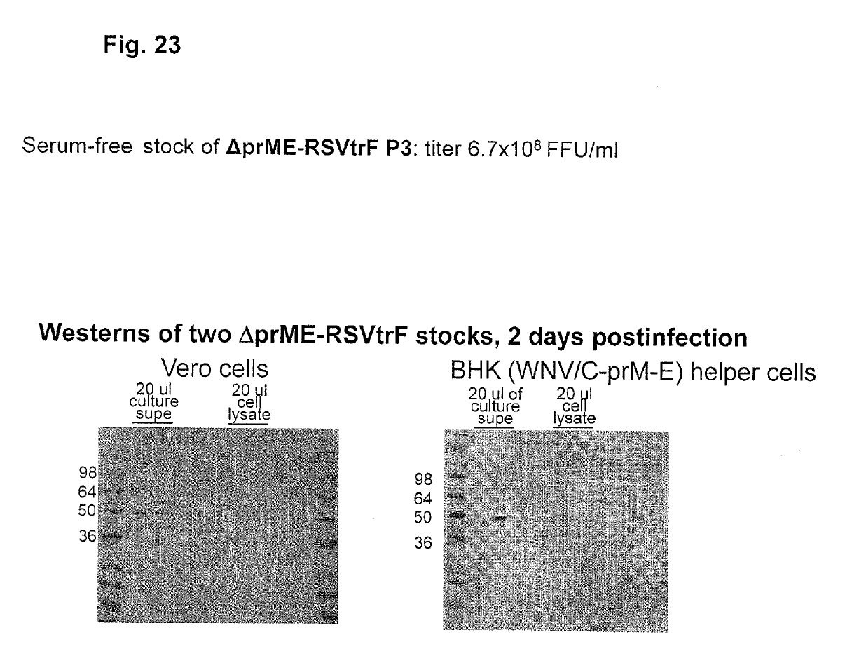

It has been demonstrated that a C-terminally truncated, secreted form of RSV F could be more immunogenic than full-length protein (Li et al., J. Exp. Med. 188:681-688, 1998). Therefore, we also cloned the available truncated RSV F gene (see FIG. 20) into the WN PIV vectors. Insertion designs were as in FIG. 13, with the only exception that the gene did not contain the sequence encoding C-terminal F protein anchor, to produce soluble form of truncated F (trF) in the lumen of the ER, which should be efficiently secreted from cells. Resulting WN(ΔC)-RSV trF, WN(ΔprME)-RSV trF, and WN(ΔCprME)-RSV trF PIVs (see Sequence Appendix 6 for the sequences of these constructs) were recovered in helper BHK-CprME(WN) cells. Results of IFA for transfected cells, performed with anti-RSV F Mab, are shown in FIG. 21. Efficient expression of trF product was observed, also demonstrating that all defective recombinant viruses were viable. Titers of PIVs as high as 2×10⁶ FFU/ml were observed in cell supernatants immediately after transfection; these are expected to further increase with passages. Importantly, similar numbers of foci were detected by both anti-RSVF and anti-WN antibodies in titration experiments in Vero cells (FIG. 22), and intensities of staining with both antibodies were comparable indicative of high-level expression of trF product. Western analysis of two ΔprME-RSVtrF stocks, two days post-infection of Vero and BHK (WNV/C-prM-E) helper cells, is shown in FIG. 23. These PIVs can be evaluated further for immunogenicity/efficacy in available animal models for RSV disease, in both s-PIV and d-PIV formulations (see below for further evaluation of ΔprME-RSVtrF).

Set forth below is the RSV amino acid sequence of the truncated construct. The chimeric West Nile/RSV-F signal peptide (ggktgiavi/melpiikanaittiliavtfcfass) is designed to be cleaved by signal protease after “ . . . fass”, releasing N-terminus of F2 “qnitee . . . ”. At the C-terminus is the sequence of autoprotease FMDV 2A fused to RSVF (nfdllklagdvesnpg). This sequence and/or only the RSV F protein portion thereof can be used in any of the vectors described herein. Further, this sequence (and/or only the RSV F protein portion thereof) can be the basis for derivation of analogs and fragments for use in the invention. Thus, sequences having percentage identities to this sequence, as described above, or fragments, as described above, can be used in the invention.

[symbol]

qniteefyqstc
savskgylsalrtgwytsvitielsnikenkcngtdakvklikqeldkyk
navtelqllmqstpaannrarrelprfmnytlnnakktnvtlskkrkrrf
lgfllgvgsaiasgiavskvlhlegevnkiksallstnkavvslsngvsv
ltskvldlknyidkqllpivnkqscsisnietviefqqknnrlleitref
svnagvttpvstymltnsellslindmpitndqkklmsnnvqivrqqsys
imsiikeevlayvvqlplygvidtpcwklhtsplcttntkegsnicltrt
drgwycnnagsvsffpladtckvqsnrvfcdtmnsltlpsevnlcnidif
npkydckimtsktdvsssvitslgaivscygktkctasnknrgiiktfsn
gcdyvsnkgvdtvsvgntlyyvnkqegkslyvkgepiinfydplvfpsde
fdasisqvnekinqslafirksdellhnvnagksttnim
nfdllklagdvesnpg

### Recombinant Poxviruses Expressing RSV F

Based on the premise that protection can be obtained using a very limited, but focused antibody response, we have shown that a live vector expressing a codon optimized anchorless RSV F confers protection against RSV infection in an appropriate animal model and thus can be suitable for an infant vaccine.

NYVAC is a highly attenuated vaccinia strain with a series of deletion of virulence-associated or host-range genes of the Copenhagen strain (Tartaglia et al., Dev. Biol. Stand. 84:159-163, 1995). It has been used in a variety of pre-clinical and clinical studies and shown to be promising. Therefore, NYVAC has been included as a delivery vehicle for a comparative vaccine evaluation.

To generate the recombinant NYVAC expressing codon-optimized anchorless RSV F, IVR (in vitro recombination) was performed with CEF cells infected by parental NYVAC at M.O.I. of 10 and transfected with donor plasmid pLNZ16. Subsequently, IVR reaction products were serially diluted ten-fold from 1:10³ to 1:10⁶, and plated on CEF cells in 100-mm plates overlaid with medium-agarose without Blue-Gal. Three days after the first overlay, a second overlay containing Blue-Gal and Neutral Red was added. Blue plaques were picked and plaque purification continued until a white plaque was available for amplification. The isolated plaque went through three amplification steps, i.e., P1, P2, and P3.

The recombinant was fully characterized at P2 to confirm identity and purity. The NYVAC recombinant was designated vP2400.

Fowlpox is a member of the avipoxvirus genus and can cause disease in chickens and turkeys. Transmission of fowlpox virus is limited to avian species, with replication in mammalian cells resulting in abortive replication. The inability of fowlpox to produce infectious virus in mammalian cells renders fowlpox a very attractive vector for human vaccine development. The safety and efficacy of fowlpox-based vaccines have been investigated in a number of clinical trials for diseases such as cancer, HIV, and malaria. Preliminary results indicate that fowlpox vaccines are safe and well tolerated, and have demonstrated both immune and clinical efficacy. This vector was also used to compare delivery systems that express the RSV F gene product, and to allow a thorough evaluation of both immune efficacy and safety in relevant animal model systems.

To generate recombinant fowlpox expressing codon-optimized anchorless RSV F, IVR was performed with CEF cells infected by a parental fowlpox at M.O.I. of 10 and transfected with the donor plasmid pLNZ15 (Paoletti, Proc. Natl. Acad. Sci. U.S.A. 93:11349-11353, 1996). The rest of the steps are the same as above. The fowlpox recombinant was designated vFP2403.

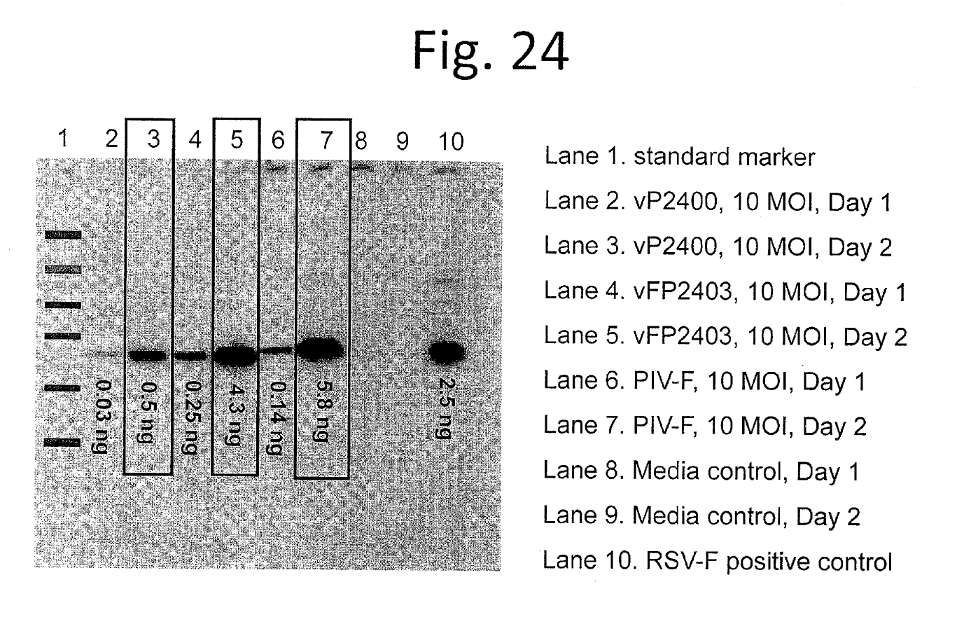

### Western Blot Analysis of PIV-mediated Expression of RSV F (See FIG. 24)

Vero cells (˜1.5×10⁶) were infected at an MOI of 10 with: Lanes 2 and 3, vP2400 (NYVAC-RSV F); Lanes 4 and 5, vFP2403 (fowlpox-RSV F); Lanes 6 and 7, PIV-F (ΔprME-RSVtrF); and Lanes 8 and 9, mock infected cells. All recombinant viruses express a codon optimized anchorless RSV F. Cell supernatants were harvested at 24 (Lanes 2, 4, 6, and 8) and 48 (Lanes 3, 5, 7, and 9) hours after infection. Equal amounts of the supernatant samples were analyzed by SDS-PAGE and the amount of RSV F present in each sample was determined using primary antibody, i.e., a mouse anti-RSV F (5353C75), followed by a goat anti-mouse IgG-horseradish peroxidase (HRP) conjugate as secondary antibody. The level of RSV F present in each sample was measured by comparison to a purified preparation of protein F from RSV-infected cells (2.5 ng, Lane 10) measured using a Kodak Imager Station 4000MM Pro. The results demonstrate that the amount of RSV F expressed in PIV-F infected Vero cells was significantly greater than that expressed by NYVAC and similar to that expressed by fowlpox.

### Immunization Studies Using PIV-F

For intramuscular immunization, Balb/c (6-8 weeks old) were injected bilaterally with 2×50 μl of PBS solution containing viral vectors expressing RSV F protein at two doses—either 10⁶ or 10⁷ PFU. Animals were boosted 4 weeks later with the same dose of the vaccine. Mice in control groups were immunized intranasally with 10⁶ PFU RSV-Long strain or intramuscularly with an FI-RSV vaccine (100 μl) prepared according to the procedures used for the 1960's trials. Four weeks after boost, mice were challenged intranasally with either 2.2×10⁶ PFU RSV-A2 (for RSVi27) or 10⁷ PFU RSV-A2 (for RSVi32).

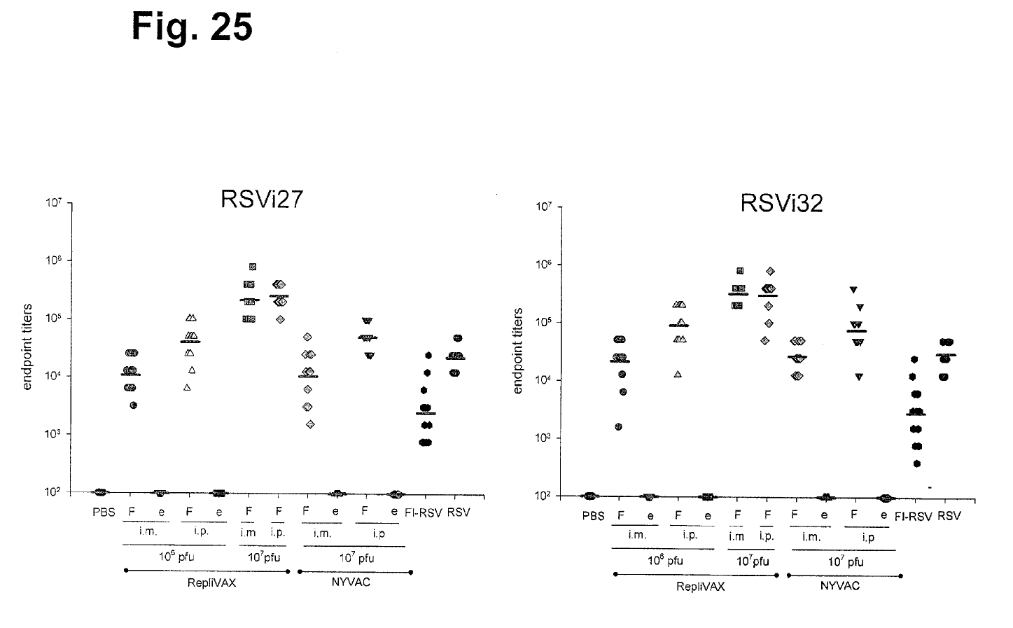

ELISA Analysis of Sera Derived from Vaccinated Mice (See FIG. 25)

Immune sera were analyzed for anti-RSV-F IgG antibody titers using ELISA, which was performed with an immunoaffinity-purified full-length RSV protein (50 ng/ml) by two-fold dilutions of immune sera. Goat anti-mouse F(ab)2 IgG (H+L) conjugated to horseradish peroxidase was used as secondary antibody. The titer is a reciprocal of the last dilution at which the OD450 was greater than 0.1 and at least twice that of a control, to which no sample was added. It can be seen from FIG. 25 that both i.m. and i.p. immunization with PIV-F generated the highest titers of IgG of the vectors tested.

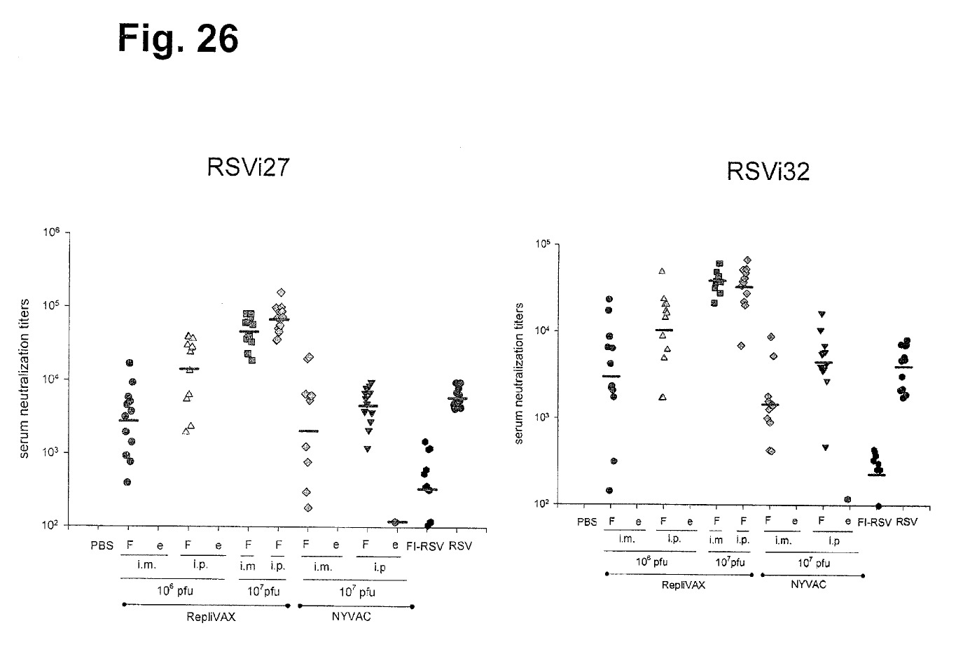

### Neutralization Assay (See FIG. 26)

Vero cells were seeded onto 24-well plates (1.5×10⁵ per well), incubated at 37° C. for two days. The neutralization reaction mixtures (serial diluted sera+virus+complement) were prepared in DMEM and incubated for 1 hour in a 37° C. shaker. The neutralization mixtures were added to the Vero cells. After a 2 hour incubation in a 37° C. shaker, the mixtures were removed and overlay media (methyl cellulose/DMEM) was added to each well. The infected Vero cells were incubated for 4 days at 37° C., then fixed with 80% methanol and stained with a primary antibody, i.e., a mouse anti-RSV F antibody (5353C75), followed by a goat anti-mouse IgG-horseradish peroxidase (HRP) conjugate as secondary antibody. The plaques were counted by eye and neutralizing titers were expressed as the dilution that caused 60% plaque reduction.

It can be seen from FIG. 26 that both i.m. and i.p. immunization with PIV-F showed the highest neutralization titers of all vectors tested.

TABLE 1
PIV prototype constructs used in platform development studies

| Construct | Genetic composition | Packaged in |
|---|---|---|
| PIV-WN | wt NY99 WN virus | WN envelope; BHK-CprME(WN) or BHK-C(WN) |
| | | helper cells (Mason et al., Virology 2006, 351: 432-43; |
| | | Widman et al., Vaccine 2008, 26: 2762-71) |
| PIV-YF/WN | Envelope (VLP): wt WN NY99 | YF 17D envelope; BHK-CprME(YF) helper cells |
| | Backbone: YF 17D | (Widman et al., Adv Virus Res. 2008, 72: 77-126) |
| PIV-WN/JE | Envelope (VLP): wt JE Nakayama | JE or WN envelope; BHK-C(WN) or BHK- |
| | Backbone: wt WN NY99 | CprME(WN) helper cells (Ishikawa et al., Vaccine |
| | | 2008, 26: 2772-8) |
| PIV-YF | YF 17D | YF 17D envelope; BHK-CprME(YF) or BHK-C(YF) |
| | | helper cells (Mason et al., Virology 2006, 351: 432-43) |

TABLE 2
Safety: Suckling mouse neurovirulence¹

| | Doses | | | |
|---|---|---|---|---|
| Construct | (log₁₀) | Mortality (%) | | AST (days)² |
| PIV-YF | 1-4 | 0/10 | (0%) | na |
| PIV-WN | 2-5 | 0/10 | (0%) | na |
| PIV-WN/JE | 1-4 | 0/11 | (0%) | na |
| PIV-YF/WN | 1-4 | 0/10-11 | (0%) | na |
| WN d-PIV | 1-4 | 0/10-11 | (0%) | na |
| YF d-PIV | 1-4 | 0/10 | (0%) | na |
| YF17D | 2 | 10/10 | (100%) | 7.6 |
| | 1 | 10/10 | (100%) | 9.3 |
| | 0 | 9/10 | (90%) | 9.9 |
| | −1 | 3/10 | (30%) | 9.6 |
| YF/JE | 4 | 9/11 | (82%) | 9.7 |
| | 3 | 7/10 | (70%) | 12.3 |
| | 2 | 3/11 | (27%) | 12 |
| | 1 | 0/11 | (0%) | na |
| YF/WN | 3 | 2/11 | (18%) | 12.5 |
| | 0-2 | 0/10-11 | (0%) | na |

¹Single dose, IC inoculation, ICR 5-day old mice, graded log doses administered.
²AST for mice that died; na, not applicable.

TABLE 3
PIV highly immunogenic and efficacious in mice¹

| | | | PRNT | PRNT | Post-challenge |
|---|---|---|---|---|---|
| Group | | Dose | Day 20 | Day 34 | mortality (%) |
| PIV-WN | | 10⁵ | 640 | 1280 | 0/8 (0%) |
| | | 10⁶ | 1280 | 2560 | 1/8 (12.5%) |
| | | 10⁶ + 10⁵ | 2560 | 2560 | 0/6 (0%) |
| YF/WN | | 10⁴ | 1280 | 2560 | 1/8 (12.5%) |
| control | | | | | |
| PIV-WN/JE | | 10⁴ | 10 | 20 | N/D |
| | | 10⁵ | 20 | 20 | N/D |
| | | 10⁵ + 10⁵ | 20 | 160 | N/D |
| YF/JE | | 10⁴ | 160 | 320 | N/D |
| control | | | | | |
| PIV-YF | | 10⁴ | <10 | <10 | 8/8 (100%) |
| | | 10⁵ | <10 | <10 | 5/7 (71%) |
| | | 10⁵ + 10⁵ | 10 | 10 | 2/5 (40%) |
| YF17D | | 10⁴ | 640 | 1280 | 0/7 (0%) |
| control | | | | | |
| Mock | WN | Diluent | N/D | 0 | 7/7 (100%) |
| control | challenge | | | | |
| | YF | Diluent | N/D | 0 | 8/8 (100%) |
| | challenge | | | | |

¹IP immunization (d0 prime, and d21 boost in select groups); challenge on d35: wt WN NY99, 3 log₁₀ PFU IP, 270 LD50; wt YF Asibi, 3 log₁₀ PFU IC, 500 LD50; N/D, not determined.

TABLE 4
PIV are immunogenic in hamsters and protect against challenge¹

| | | | | POST-CHALLENGE | | |
|---|---|---|---|---|---|---|
| | | | PRNT | | | Peak viremia |
| Group | | Dose(s) | Day 38 | Mortality | Morbidity | (log) |
| PIV-WN | | 10⁵ | 320 | 0/5 (0%) | 0/5 (0%) | 2.3 |
| | | 10⁶ | 640 | 0/5 (0%) | 0/5 (0%) | 1.8 |
| | | 10⁶ + 10⁵ | 1280 | 0/5 (0%) | 0/5 (0%) | <1.3 |
| YF/WN control | | 10⁴ | ≧2560 | 0/5 (0%) | 0/5 (0%) | <1.3 |
| PIV-WN/JE | | 10⁴ | 20 | 2/5 (40%) | 2/5 (40%) | 2.2 |
| | | 10⁵ + 10⁵ | 40 | 0/5 (0%) | 0/5 (0%) | <1.3 |
| YF/JE control | | 10⁴ | 2560 | 0/5 (0%) | 0/5 (0%) | 1.3 |
| PIV-YF | | 10⁴ | <10 | 1/3 (33%) | 3/3 (100%) | 8.3 |
| | | 10⁵ | <10 | 1/5 (20%) | 4/5 (80%) | 8.3 |
| | | 10⁵ + 10⁵ | 20 | 0/4 (0%) | 0/4 (0%) | 2.5 |
| YF17D control | | 10⁴ | ≧2560 | 0/4 (0%) | 0/4 (0%) | <1.3 |
| Mock control | WN challenge | Diluent | <10 | 3/4 (75%) | 4/4 (100%) | 4.0 |
| | YF challenge | Diluent | <10 | 1/4 (25%) | 4/4 (100%) | 8.4 |
| | JE challenge | Diluent | <10 | 2/5 (40%) | 2/5 (40%) | 3.0 |

¹Syrian hamsters, SC inoculation (d0, and d21 in select groups); challenge (d39): wt WN NY385/99 6 log₁₀ PFU IP, wt JE Nakayama 5.8 log₁₀ PFU IC, or hamster-adapted YF Asibi 7 log₁₀ PFU IP (McArthur et al., J. Virol. 77: 1462-1468, 2003; McArthur et al., Virus Res. 110: 65-71, 2005).

TABLE 5
Immunization of hamsters with PIV: comparison of SC and IP routes

| | | |
|---|---|---|
| PRNT | | PRNT |
| Day 20-21 | Boost | Day 34-38 |

| Inoculums | SC | IP | (log₁₀) | SC | IP |
|---|---|---|---|---|---|
| PIV-WN | 40 | 320 | 5 | 1280 | 1280 |
| PIV-YF/WN | 10 | 320 | 5 | 160 | 1280 |
| PIV-WN/JE | 10 | 80 | 5 | 40 | 640 |
| PIV-YF | <10 | 10 | 5 | 20 | 80 |

TABLE 6
Immune responses to PIV cocktails (mice)¹

| | | PRNT Day 20 | | PRNT Day 34 | |
|---|---|---|---|---|---|
| Group | Dose | Anti-JE | Anti-WN | Anti-JE | Anti-WN |
| PIV-WN/JE + | 10⁵ + 10⁵ | 20 | 320 | 640 | 5120 |
| RV-WN | | | | | |
| PIV-WN/JE alone | 10⁵ | 80 | <10 | 160 | 20 |
| PIV-WN alone | 10⁵ | <10 | 640 | <10 | 5120 |
| Mock | — | <10 | <10 | <10 | <10 |

¹C57/BL6 mice, IP inoculations on days 0 and 21; pooled serum PRNT titers.

TABLE 7
Neurovirulence (IC inoculation) and neuroinvasiveness (IP inoculation)
of PIV-TBE and YF/TBE vaccine constructs in adult ICR mice

| | Neurovirulence (IC route) | | | | Neuroinvasiveness (IP route) | | |
|---|---|---|---|---|---|---|---|
| | Dose(s) | Mortality | | AST, | Dose(s) | Mortality | AST, |
| Construct | (log₁₀) | (%) | | days¹ | (log₁₀) | (%) | days¹ |
| PIV-Hypr p39 | 5 | 0/7 | (0%) | na | 5 | 0/16 (0%) | na |
| PIV-Hypr p40 | 5 | 0/6 | (0%) | na | 5 | 0/16 (0%) | na |
| YF/Hypr p42 | 4 | 8/8 | (100%) | 6.3 | 5 | 6/8 (75%) | 13.3 |
| | 3 | 8/8 | (100%) | 6.4 | | | |
| | 2 | 8/8 | (100%) | 7.4 | | | |
| YF/LGT p43 | 4 | 8/8 | (100%) | 7.9 | 5 | 0/8 (0%) | na |
| | 3 | 8/8 | (100%) | 7.6 | | | |
| | 2 | 8/8 | (100%) | 8.4 | | | |
| YF/Hypr p45 | 4 | 8/8 | (100%) | 6.1 | 5 | 5/8 (62.5%) | 11.2 |
| | 3 | 8/8 | (100%) | 6.6 | | | |
| | 2 | 8/8 | (100%) | 6.8 | | | |
| YF/Hypr dC2 p59 | 4 | 8/8 | (100%) | 6.6 | 5 | 0/8 (0%) | na |
| | 3 | 8/8 | (100%) | 7.4 | | | |
| | 2 | 8/8 | (100%) | 8.1 | | | |
| YF 17D | 3 | 8/8 | (100%) | 9 | 5 | 0/8 (0%) | na |
| | 2 | 7/8 | (87.5%) | 9.6 | | | |
| | 1 | 4/8 | (50%) | 10 | | | |
| Mock (diluent) | none | 0/8 | (0%) | na | none | 0/8 (0%) | na |

¹AST for mice that died.

TABLE 8
Neutralizing antibody titers (PRNT₅₀) in mice immunized IP
(determined against wt TBE virus Hypr), and protection from
challenge (postchallenge observation, day 9)

| | | PRNT₅₀ titer, | | | Postchallenge |
|---|---|---|---|---|---|
| | Dose(s), | individ. | | PRNT₅₀ | mortality (%) |
| Immunogen | log₁₀ | samples¹ | | GMT | on day 9² |
| PIV-Hypr p39, | 5 | 1746 | (2) | 665 | 0/8 (0%) |
| 1 dose | | 1187 | (2) | | |
| | | 164 | (2) | | |
| | | 574 | (2) | | |
| PIV-Hypr p39, | 5 + 5 | 16229 | (2) | 10,584 | 0/8 (0%) |
| 2 doses | | 12928 | (2) | | |
| | | 12927 | (2) | | |
| | | 4627 | (2) | | |
| PIV-Hypr p40, | 5 | <10 | (2) | 15 | 6/8 (75%) |
| 1 dose | | <10 | (2) | | |
| | | 18 | (2) | | |
| | | 33 | (2) | | |
| PIV-Hypr p40, | 5 + 5 | 169 | (2) | 153 | 1/8 (12.5%) |
| 2 doses | | 638 | (2) | | |
| | | 26 | (2) | | |
| | | 192 | (2) | | |
| YF/Hypr p42 | 5 | 9210 | (1) | 6,085 | 0/2 (0%) |
| | | 4020 | (1) | | |
| YF/LGT p43 | 5 | 123 | (2) | 64 | 1/8 (12.5%) |
| | | 32 | (2) | | |
| | | 96 | (2) | | |
| | | 45 | (2) | | |
| YF/Hypr p45 | 5 | 292 | (2) | 68 | 0/3 (0%) |
| | | 16 | (1) | | |
| YF/Hypr dC2 p59 | 5 | 194 | (2) | 68 | 0/8 (0%) |
| | | 93 | (2) | | |
| | | 45 | (2) | | |
| | | 26 | (2) | | |
| Killed human TBE | 1/20 | 19 | (2) | 12 | 1/8 (12.5%) |
| vaccine, 1 | | <10 | (2) | | |
| dose (at 1/20 of | | 13 | (2) | | |
| human dose) | | <10 | (2) | | |
| Killed human TBE | 1/20 + | 3435 | (2) | 1,496 | 0/6 (0%) |
| vaccine, 2 doses | 1/20 | 1267 | (2) | | |
| (each at | | 770 | (2) | | |
| 1/20 of human dose) | | | | | |
| YF 17D control | 5 | <10 | (4) | <10 | 5/8 (62.5%) |
| | | 11 | (4) | | |
| Mock | none | <10 | (4) | <10 | 4/8 (50%) |
| | | <10 | (4) | | |

¹Numbers in parenthesis correspond to number of mice in each pooled serum sample tested.
²Mortalities on day 9 are shown.

TABLE 9
Examples of published attenuating E protein mutations that can be used for
attenuation of chimeric TBE LAV candidates

| Residue | Domain | Comments | Attenuation in | Reference |
|---|---|---|---|---|
| N52R | II | DI-DII hinge, possibly involved in hinge | JE, YF | Hasegawa et al, 1992, |
| | | motion required for fusion activation | | Schlesinger et al, 1996 |
| E84K | II | conserved, E in TBE, K/R in others, | TBE | Labuda et al, 1994 |
| | | attenuated by passage in ixodes ricinus | | |
| | | ticks, DII contains flavivirus cross reactive | | |
| | | epitopes | | |
| E85K | II | conserved, E in TBE, K/R in others, | JE | Wu et al, 1997 |
| | | attenuation obtained as plaque variants in | | |
| | | Vero cells, DII contains flavivirus cross | | |
| | | reactive epitopes | | |
| H104K | II | within highly conserved fusion peptide (aa | TBE | Rey et al, 1995 |
| | | 98-113), H in TBE, G in others | | |
| L107F | II | within highly conserved fusion peptide (aa | TBE, JE, WN | Rey et al, 1995, Arroyo |
| | | 98-113), L in all flaviviruses, F in | | et al, 1999, 2004 |
| | | attenuated JE | | |
| T123K | II | DI-DII hinge, T in TBE, A in KFD | TBE | Holzmann et al, 1997 |
| K126E | II | DI-DII hinge, K in TBE, E in D-2 | DEN2 | Bray, 98 |
| K136E | II | DI-DII hinge, K in TBE and JE, E in D-2 | JE | |
| N154L(Y) | I | glycosylation site, packed with conserved | DEN2, DEN4, YF | Guirakhoo et al, 1993, Pletnev et |
| | | H 104, involved in fusion. | | al, 1993, Kawano et al, 1993, |
| | | | | Jennings et al, 1994 |
| K171E | I | external edge of DI, involved in fusion | TBE | Mandl, 1989, Holzmann, 1997 |
| I173T | | external edge of DI, involved in fusion | YF | Chambers and Nickells 2001 |
| D181Y | | DI-DII hinge | TBE | Holzmann et al, 1997 |
| K204R | | Lining Hydrophobic pocket, involve in | DEN1, DEN3 | Guirakhoo et al, 2004 |
| | | fusion | | |
| P272S | II | highly conserved, junction of one the of 2 | JE | Cecilia et al, 1991 |
| | | alpha helices | | |
| G308N | III | cell attachment, DKT in TBE, EGS in KFD, | LI | Jiang et al, 1993, Gao et al, 1994 |
| | | T-X in others, change to N produced | | |
| | | glycosylation site in LI and reduced | | |
| | | virulence, N-X-T/S glycosylation motif | | |
| S310K | III | putative cell attachment, change from E to | JE | Jiang et al, 1993, Gao et al, |
| | | G in JE reduced virulence | | 1994, Wu et al, 1997 |
| K311E | III | highly conserved, putative cell attachment | TBE, YF | Rey et al, 1995, Jennings, 1994 |
| T333L | III | putative cell attachment | YF, LGT | Raynman et al, 1998 |
| G334K | III | putative cell attachment | YF | Chambers and Nickells, 2001 |
| S335K | III | putative cell attachment | JE | Wu et al, 1997 |
| K336D | III | putative cell attachment | JE | Cecilia and Gould, 1991 |
| P337D | III | putative cell attachment | JE | Cecilia and Gould, 1991 |
| G368R | III | putative cell attachment | TBE, JE | Holzman et al 1997, Hasegawa et |
| | | | | al 1992 |
| Y384H | III | change to H attenuated TBE, putative cell | TBE | Holzmann et al, 1990 |
| | | attachment, −3 position to deleted RGD in | | |
| | | TBE | | |
| V385R | III | conserved, −2 position to deleted RGD in | D2 | Hiramatsu et al, 1996, Lobigs, 90 |
| | | TBE, putative cell attachment | | |
| G386R | III | highly conserved, −1 position to deleted | D2, MVE | Hiramatsu, 96, Lobigs et al, 1990 |
| | | RGD in TBE, putative cell attachment | | |
| E387R | III | conserved, +2 position to deleted RGD in | D2, MVE | Hiramatsu, 1996, Lobigs et al, |
| | | TBE, putative cell attachment | | 1990 |
| F403K | none | highly conserved, C-terminal region not | D-2, D-4 | Kawano et al, 1993, Bray et al, |
| | | included in crystal structure sE | | 1998 |
| H438Y | None | highly conserved, C-terminal region not | LGT | Campbell and Pletnev 2000 |
| | | included in crystal structure sE | | |
| H496R | none | highly conserved, C-terminal region not | TBE | Gritsun et al, 2001 |
| | | included in crystal structure sE | | |

References:
Hasegawa et al., Virology 191(1): 158-165;
Schlesinger et al., J. Gen. Virol. 1996, 77 (Pt 6): 1277-1285, 1996;
Labuda et al., Virus Res. 31(3): 305-315, 1994;
Wu et al., Virus Res. 51(2): 173-181, 1997;
Holzmann et al., J. Gen. Virol. 78 (Pt 1): 31-37, 1997;
Bray et al., J. Virol. 72(2): 1647-1651, 1998;
Guirakhoo et al., Virology 194(1): 219-223, 1993;
Pletnev et al., J. Virol. 67(8): 4956-4963, 1993;
Kawano et al., J. Virol. 67(11): 6567-6575, 1993;
Jennings et al., J. Infect. Dis. 169(3): 512-518, 1994;
Mandl et al., J. Virol. 63(2): 564-571, 1989;
Chambers et al., J. Virol. 75(22): 10912-10922, 2001;
Cecilia et al., Virology 181(1): 70-77, 1991;
Jiang et al., J. Gen. Virol. 74 (Pt 5): 931-935, 1993;
Gao et al., J. Gen. Virol. 75 (Pt 3): 609-614, 1994;
Holzmann et al., J. Virol. 64(10): 5156-5159, 1990;
Hiramatsu et al., Virology 224(2): 437-445, 1996;
Lobigs et al., Virology 176(2): 587-595, 1990;
Campbell et al., Virology 269(1): 225-237, 2000;
Gritsun et al., J. Gen. Virol. 82(Pt 7): 1667-1675, 2001.

### Other Embodiments

All publications, patent applications, and patents mentioned in this specification are incorporated herein by reference in their entirety as if each individual publication, patent application, or patent were specifically and individually indicated to be incorporated by reference.

Various modifications and variations of the described viruses, vectors, compositions, and methods of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the fields of medicine, pharmacology, or related fields are intended to be within the scope of the invention. Use of singular forms herein, such as “a” and “the,” does not exclude indication of the corresponding plural form, unless the context indicates to the contrary. Similarly, use of plural terms does not exclude indication of a corresponding singular form. Other embodiments are within the scope of the following claims.

What is claimed is:

SEQUENCE APPENDIX 1
CV-TBEV Hypr or CV-LGT E5 with YFV/TBEV chimeric signal (p42, p59, and p43 constructs)

| | |
|---|---|
| | YF17D partial signal |
| | --------------------------------------- |
| | TBEV partial signal |
| | --------------------------- |
| | Hypr or LGT |
| | C protein YFLTD BS prM protein |
| | -------------- ------------- |
| | R K R K S H D V L T V Q F L I L G M L G M T I A A T V R |
| 401 | A GGAAACGCCG TTCCCATGAT GTTCTGACTG TGCAATTCCT AATTTTGGGC ATGCTGGGCA TGACAATCGC AGCTACGGTT CGC |
| | T CCTTTGCGGC AAGGGTACTA CAAGACTGAC ACGTTAAGGA TTAAAACCCG TACGACCCGT ACTGTTAGCG TCGATGCCAA GCG |

CV-TBEV Hypr with YFV/WNV chimeric signal (p45)

| | |
|---|---|
| | C protein YF17D WNV partial signal |
| | -------------- -------------------------- |
| | YF 17D partial signal Hypr prM protein |
| | ---------------------------------------- -------------- |
| | R K R R S H D V L T V Q F L I L G M L A C V G A A T V R |
| 401 | A GGAAACGCCG TTCCCATGAT GTTCTGACTG TGCAATTCCT AATTTTGGGC ATGCTGGCTT GTGTCGGAGC AGCTACCGTG CGA |
| | T CCTTTGCGGC AAGGGTACTA CAAGACTGAC ACGTTAAGGA TTAAAACCCG TACGACCGAA CACAGCCTCG TCGATGGCAC GCT |

RV-WNV/TBEV Hypr with TBEV signal (p39)

| | |
|---|---|
| | TBEV signal |
| | ------------------------------------------------------------------ |
| | WNV C protein Hypr prM protein |
| | -------------- ------------- |
| | Q K K R G G T D W M S W L L V I G M L G M T I A A T V R |
| 201 | CAAAAGAAA CGGGGGGGAA CAGACTGGAT GAGCTGGCTG CTCGTAATCG GCATGCTGGG CATGACAATC GCAGCTACGG TTCGC |
| | GTTTTCTTT GCCCCCCCTT GTCTGACCTA CTCGACCGAC GAGCATTAGC CGTACGACCC GTACTGTTAG CGTCGATGCC AAGCG |

RV-WNV/TBEV Hypr with WNV signal (p40)

| | |
|---|---|
| | WNV signal |
| | ----------------------------------------------------------- |
| | WNV C protein Hypr prM protein |
| | ------------- ------------- |
| | Q K K R G G K T G I A V M I G M L A C V G A A T V R |
| 201 | CAAAAGAAA CGCGGGGGAA AGACAGGCAT AGCTGTGATG ATAGGCATGC TGGCTTGTGT CGGAGCAGCT ACCGTGCGA |
| | GTTTTCTTT GCGCCCCCTT TCTGTCCGTA TCGACACTAC TATCCGTACG ACCGAACACA GCCTCGTCGA TGGCACGCT |

SEQUENCE APPENDIX 2
CV-TBEV Hypr with YFV/TBEV chimeric signal (p42)

| | |
|---|---|
| | 5′ UTR |
| | ---------------------------------------------------------------------------------------- |
| 1 | AGTAAATCCT GTGTGCTAAT TGAGGTGCAT TGGTCTGCAA ATCGAGTTGC TAGGCAATAA ACACATTTGG ATTAATTTTA |
| | TCATTTAGGA CACACGATTA ACTCCACGTA ACCAGACGTT TAGCTCAACG ATCCGTTATT TGTGTAAACC TAATTAAAAT |
| | 5′ UTR |
| | --------------------- |
| | ATCGTTCGTT GAGCGATTAG |
| | TAGCAAGCAA CTCGCTAATC |
| | 5′ UTR |
| | ------------------- |
| | C protein |
| | --------------------------------------------------------------------- |
| | M S G R K A Q G K T L G V N M V R R G V R |
| 101 | CAGAGAACTG ACCAGAACAT GTCTGGTCGT AAAGCTCAGG GAAAAACCCT GGGCGTCAAT ATGGTACGAC GAGGAGTTCG |
| | GTCTCTTGAC TGGTCTTGTA CAGACCAGCA TTTCGAGTCC CTTTTTGGGA CCCGCAGTTA TACCATGCTG CTCCTCAAGC |
| | C protein |
| | --------------------- |
| | S L S N K I K • |
| | CTCCTTGTCA AACAAAATAA |
| | GAGGAACAGT TTGTTTTATT |
| | C protein |
| | ---------------------------------------------------------------------------------------- |
| | • Q K T K Q I G N R P G P S R G V Q G F I F F F L F N |
| 201 | AACAAAAAAC AAAACAAATT GGAAACAGAC CTGGACCTTC AAGAGGTGTT CAAGGATTTA TCTTTTTCTT TTTGTTCAAC |
| | TTGTTTTTTG TTTTGTTTAA CCTTTGTCTG GACCTGGAAG TTCTCCACAA GTTCCTAAAT AGAAAAAGAA AAACAAGTTG |
| | C protein |
| | --------------------- |
| | I L T G K K I • |
| | ATTTTGACTG GAAAAAAGAT |
| | TAAAACTGAC CTTTTTTCTA |
| | C protein |
| | ---------------------------------------------------------------------------------------- |
| | • T A H L K R L W K M L D P R Q G L A V L R K V K R V V |
| 301 | CACAGCCCAC CTAAAGAGGT TGTGGAAAAT GCTGGACCCA AGACAAGGCT TGGCTGTTCT AAGGAAAGTC AAGAGAGTGG |
| | GTGTCGGGTG GATTTCTCCA ACACCTTTTA CGACCTGGGT TCTGTTCCGA ACCGACAAGA TTCCTTTCAG TTCTCTCACC |
| | C protein |
| | --------------------- |
| | A S L M R G |
| | TGGCCAGTTT GATGAGAGGA |
| | ACCGGTCAAA CTACTCTCCT |
| | YF17D partial signal |
| | --------------------------------------- |
| | TBEV partial signal |
| | --------------------------- |
| | C protein |
| | ---------------------------- |
| | L S S R K R R S H D V L T V Q F L I L G M L G M T I A |
| 401 | TTGTCCTCAA GGAAACGCCG TTCCCATGAT GTTCTGACTG TGCAATTCCT AATTTTGGGC ATGCTGGGCA TGACAATCGC A |
| | AACAGGAGTT CCTTTGCGGC AAGGGTACTA CAAGACTGAC ACGTTAAGGA TTAAAACCCG TACGACCCGT ACTGTTAGCG T |
| | prM protein |
| | -------------------- |
| | A T V R K E R • |
| | GCTACGGTT CGCAAGGAAA |
| | CGATGCCAA GCGTTCCTTT |
| | prM protein |
| | ---------------------------------------------------------------------------------------- |
| | • D G S T V I R A E G K D A A T Q V R V E N G T C V I |
| 501 | GAGACGGCAG TACGGTCATA CGCGCGGAAG GTAAGGATGC CGCTACCCAA GTGAGAGTGG AAAATGGTAC CTGCGTCATT |
| | CTCTGCCGTC ATGCCAGTAT GCGCGCCTTC CATTCCTACG GCGATGGGTT CACTCTCACC TTTTACCATG GACGCAGTAA |
| | prM protein |
| | --------------------- |
| | L A T D M G S • |
| | CTGGCCACCG ACATGGGCTC |
| | GACCGGTGGC TGTACCCGAG |
| | prM protein |
| | ---------------------------------------------------------------------------------------- |
| | • W C D D S L S Y E C V T I D Q G E E P V D V D C F C R |
| 601 | TTGGTGTGAT GATAGCCTTT CTTATGAGTG CGTAACCATA GATCAAGGTG AGGAACCTGT TGACGTTGAT TGCTTCTGCC |
| | AACCACACTA CTATCGGAAA GAATACTCAC GCATTGGTAT CTAGTTCCAC TCCTTGGACA ACTGCAACTA ACGAAGACGG |
| | prM protein |
| | --------------------- |
| | N V D G V Y |
| | GAAACGTGGA TGGGGTGTAT |
| | CTTTGCACCT ACCCCACATA |
| | prM protein |
| | ---------------------------------------------------------------------------------------- |
| | L E Y G R C G K Q E G S R T R R S V L I P S H A Q G E |
| 701 | CTCGAATATG GACGGTGTGG TAAACAAGAA GGAAGCAGAA CCAGACGCTC AGTGCTTATA CCCTCCCACG CTCAAGGAGA |
| | GAGCTTATAC CTGCCACACC ATTTGTTCTT CCTTCGTCTT GGTCTGCGAG TCACGAATAT GGGAGGGTGC GAGTTCCTCT |
| | prM protein |
| | --------------------- |
| | L T G R G H K • |
| | GCTGACCGGA CGGGGACATA |
| | CGACTGGCCT GCCCCTGTAT |
| | prM protein |
| | ---------------------------------------------------------------------------------------- |
| | • W L E G D S L R T H L T R V E G W V W K N R L L A L |
| 801 | AATGGTTGGA GGGCGACTCA CTCCGAACAC ATTTGACCCG CGTCGAGGGC TGGGTCTGGA AAAATCGGCT GTTGGCCCTC |
| | TTACCAACCT CCCGCTGAGT GAGGCTTGTG TAAACTGGGC GCAGCTCCCG ACCCAGACCT TTTTAGCCGA CAACCGGGAG |
| | prM protein |
| | --------------------- |
| | A M V T V V W • |
| | GCTATGGTGA CAGTCGTTTG |
| | CGATACCACT GTCAGCAAAC |
| | Hypr E |
| | Protein |
| | -------- |
| | prM protein |
| | -------------------------------------------------------------------------------- |
| | • L T L E S V V T R V A V L V V L L C L A P V Y A S R C |
| 901 | GCTCACGCTG GAGTCTGTGG TTACTCGCGT GGCAGTGCTG GTGGTGCTCC TCTGTCTTGC CCCTGTCTAC GCGTCCAGGT |
| | CGAGTGCGAC CTCAGACACC AATGAGCGCA CCGTCACGAC CACCACGAGG AGACAGAACG GGGACAGATG CGCAGGTCCA |
| | Hypr E protein |
| | --------------------- |
| | T H L E N R |
| | GTACTCATTT GGAAAACAGA |
| | CATGAGTAAA CCTTTTGTCT |
| | Hypr E protein |
| | ---------------------------------------------------------------------------------------- |
| | D F V T G T Q G T T R V T L V L E L G G C V T I T A E |
| 1001 | GATTTTGTCA CCGGCACCCA GGGGACGACT CGGGTAACCC TGGTGCTTGA ACTGGGTGGT TGCGTTACTA TTACCGCTGA |
| | CTAAAACAGT GGCCGTGGGT CCCCTGCTGA GCCCATTGGG ACCACGAACT TGACCCACCA ACGCAATGAT AATGGCGACT |
| | Hypr E protein |
| | --------------------- |
| | G K P S M D V • |
| | GGGCAAACCC TCTATGGATG |
| | CCCGTTTGGG AGATACCTAC |
| | Hypr E protein |
| | ---------------------------------------------------------------------------------------- |
| | • W L D A I Y Q E N P A Q T R E Y C L H A K L S D T K |
| 1101 | TGTGGCTGGA TGCAATCTAT CAGGAGAATC CCGCACAAAC CAGGGAATAT TGCCTTCACG CAAAGCTGTC CGATACAAAG |
| | ACACCGACCT ACGTTAGATA GTCCTCTTAG GGCGTGTTTG GTCCCTTATA ACGGAAGTGC GTTTCGACAG GCTATGTTTC |
| | Hypr E protein |
| | --------------------- |
| | V A A R C P T • |
| | GTCGCGGCTA GGTGCCCAAC |
| | CAGCGCCGAT CCACGGGTTG |
| | Hypr E protein |
| | ---------------------------------------------------------------------------------------- |
| | • M G P A T L A E E H Q G G T V C K R D Q S D R G W G N |
| 1201 | AATGGGACCG GCCACCCTGG CGGAGGAACA TCAGGGAGGT ACAGTGTGCA AACGGGACCA GAGTGATAGA GGCTGGGGTA |
| | TTACCCTGGC CGGTGGGACC GCCTCCTTGT AGTCCCTCCA TGTCACACGT TTGCCCTGGT CTCACTATCT CCGACCCCAT |
| | Hypr E protein |
| | --------------------- |
| | H C G L F G |
| | ATCACTGCGG CCTGTTCGGC |
| | TAGTGACGCC GGACAAGCCG |
| | Hypr E protein |
| | ---------------------------------------------------------------------------------------- |
| | K G S I V A C V K A A C E A K K K A T G H V Y D A N K |
| 1301 | AAAGGAAGTA TTGTCGCTTG CGTCAAGGCA GCCTGTGAGG CCAAAAAGAA GGCTACTGGG CACGTCTATG ACGCCAACAA |
| | TTTCCTTCAT AACAGCGAAC GCAGTTCCGT CGGACACTCC GGTTTTTCTT CCGATGACCC GTGCAGATAC TGCGGTTGTT |
| | Hypr E protein |
| | --------------------- |
| | I V Y T V K V • |
| | GATCGTTTAT ACAGTGAAAG |
| | CTAGCAAATA TGTCACTTTC |
| | Hypr E protein |
| | ---------------------------------------------------------------------------------------- |
| | • E P H T G D Y V A A N E T H S G R K T A S F T V S S |
| 1401 | TGGAACCACA CACAGGGGAT TACGTGGCGG CCAACGAGAC TCATTCCGGT CGCAAAACGG CCAGCTTCAC CGTGTCATCC |
| | ACCTTGGTGT GTGTCCCCTA ATGCACCGCC GGTTGCTCTG AGTAAGGCCA GCGTTTTGCC GGTCGAAGTG GCACAGTAGG |
| | Hypr E protein |
| | --------------------- |
| | E K T I L T M • |
| | GAAAAGACCA TCCTCACTAT |
| | CTTTTCTGGT AGGAGTGATA |
| | Hypr E protein |
| | ---------------------------------------------------------------------------------------- |
| | • G E Y G D V S L L C R V A S G V D L A Q T V I L E L D |
| 1501 | GGGGGAGTAT GGCGACGTTT CTCTGCTCTG CCGGGTGGCT AGCGGAGTCG ACCTGGCCCA GACAGTCATC CTGGAACTGG |
| | CCCCCTCATA CCGCTGCAAA GAGACGAGAC GGCCCACCGA TCGCCTCAGC TGGACCGGGT CTGTCAGTAG GACCTTGACC |
| | Hypr E protein |
| | --------------------- |
| | K T V E H L |
| | ATAAAACAGT TGAGCATCTG |
| | TATTTTGTCA ACTCGTAGAC |
| | Hypr E protein |
| | ---------------------------------------------------------------------------------------- |
| | P T A W Q V H R D W F N D L A L P W K H E G A R N W N |
| 1601 | CCTACCGCTT GGCAGGTGCA CAGGGATTGG TTTAACGACC TTGCCCTGCC ATGGAAACAT GAAGGAGCGA GAAACTGGAA |
| | GGATGGCGAA CCGTCCACGT GTCCCTAACC AAATTGCTGG AACGGGACGG TACCTTTGTA CTTCCTCGCT CTTTGACCTT |
| | Hypr E protein |
| | --------------------- |
| | N A E R L V E • |
| | TAATGCAGAG CGACTCGTAG |
| | ATTACGTCTC GCTGAGCATC |
| | Hypr E protein |
| | ---------------------------------------------------------------------------------------- |
| | • F G A P H A V K M D V Y N L G D Q T G V L L K A L A |
| 1701 | AATTCGGTGC CCCTCATGCC GTGAAGATGG ACGTCTACAA TCTGGGTGAT CAGACCGGCG TTCTCCTTAA AGCTCTCGCT |
| | TTAAGCCACG GGGAGTACGG CACTTCTACC TGCAGATGTT AGACCCACTA GTCTGGCCGC AAGAGGAATT TCGAGAGCGA |
| | Hypr E protein |
| | --------------------- |
| | G V P V A H I • |
| | GGCGTACCAG TTGCCCACAT |
| | CCGCATGGTC AACGGGTGTA |
| | Hypr E protein |
| | ---------------------------------------------------------------------------------------- |
| | • E G T K Y H L K S G H V T C E V G L E K L K M K G L T |
| 1801 | CGAAGGAACG AAGTACCACC TGAAGTCAGG CCATGTAACT TGCGAGGTGG GCCTGGAGAA GTTGAAAATG AAAGGTCTTA |
| | GCTTCCTTGC TTCATGGTGG ACTTCAGTCC GGTACATTGA ACGCTCCACC CGGACCTCTT CAACTTTTAC TTTCCAGAAT |
| | Hypr E protein |
| | --------------------- |
| | Y T M C D K |
| | CGTACACAAT GTGTGACAAG |
| | GCATGTGTTA CACACTGTTC |
| | Hypr E protein |
| | ---------------------------------------------------------------------------------------- |
| | T K F T W K R A P T D S G H D T V V M E V T F S G T K |
| 1901 | ACCAAGTTCA CATGGAAGAG GGCCCCCACA GATAGCGGCC ACGATACTGT GGTGATGGAG GTGACCTTTT CTGGAACAAA |
| | TGGTTCAAGT GTACCTTCTC CCGGGGGTGT CTATCGCCGG TGCTATGACA CCACTACCTC CACTGGAAAA GACCTTGTTT |
| | Hypr E protein |
| | --------------------- |
| | P C R I P V R • |
| | ACCCTGCAGA ATACCCGTGC |
| | TGGGACGTCT TATGGGCACG |
| | Hypr E protein |
| | ---------------------------------------------------------------------------------------- |
| | • A V A H G S P D V N V A M L I T P N P T I E N N G G |
| 2001 | GGGCTGTAGC TCACGGATCT CCCGATGTCA ATGTTGCTAT GCTGATTACA CCTAACCCTA CCATCGAGAA TAACGGTGGT |
| | CCCGACATCG AGTGCCTAGA GGGCTACAGT TACAACGATA CGACTAATGT GGATTGGGAT GGTAGCTCTT ATTGCCACCA |
| | Hypr E protein |
| | --------------------- |
| | G F I E M Q L • |
| | GGTTTTATTG AGATGCAGCT |
| | CCAAAATAAC TCTACGTCGA |
| | Hypr E protein |
| | ---------------------------------------------------------------------------------------- |
| | • P P G D N I I Y V G E L S Y Q W F Q K G S S I G R V F |
| 2101 | TCCGCCAGGC GATAACATCA TCTACGTGGG CGAACTCTCT TACCAGTGGT TTCAGAAAGG GAGTTCAATT GGGCGGGTCT |
| | AGGCGGTCCG CTATTGTAGT AGATGCACCC GCTTGAGAGA ATGGTCACCA AAGTCTTTCC CTCAAGTTAA CCCGCCCAGA |
| | Hypr E protein |
| | --------------------- |
| | Q K T K K G |
| | TCCAAAAAAC GAAGAAGGGA |
| | AGGTTTTTTG CTTCTTCCCT |
| | Hypr E protein |
| | ---------------------------------------------------------------------------------------- |
| | I E R L T V I G E H A W D F G S A G G F L S S I G K A |
| 2201 | ATCGAACGAT TGACGGTTAT CGGCGAGCAC GCATGGGATT TTGGTTCCGC AGGGGGATTC CTGTCTTCTA TTGGTAAGGC |
| | TAGCTTGCTA ACTGCCAATA GCCGCTCGTG CGTACCCTAA AACCAAGGCG TCCCCCTAAG GACAGAAGAT AACCATTCCG |
| | Hypr E protein |
| | --------------------- |
| | L H T V L G G • |
| | ACTGCATACC GTGCTGGGGG |
| | TGACGTATGG CACGACCCCC |
| | Hypr E protein |
| | ---------------------------------------------------------------------------------------- |
| | • A F N S I F G G V G F L P K L L L G V A L A W L G L |
| 2301 | GCGCATTCAA TTCTATTTTC GGGGGCGTGG GGTTCCTGCC TAAACTCCTG CTGGGAGTAG CCCTGGCCTG GTTGGGACTG |
| | CGCGTAAGTT AAGATAAAAG CCCCCGCACC CCAAGGACGG ATTTGAGGAC GACCCTCATC GGGACCGGAC CAACCCTGAC |
| | Hypr E protein |
| | --------------------- |
| | N M R N P T M • |
| | AATATGCGGA ATCCGACGAT |
| | TTATACGCCT TAGGCTGCTA |
| | Hypr E protein |
| | ------------------------------------------------------------------- |
| | NS1 gene of YF17D |
| | --------------------- |
| | • S M S F L L A G V L V L A M T L G V G A D Q G C A I N |
| 2401 | GTCCATGTCA TTCCTCTTGG CCGGCGTGCT TGTACTGGCC ATGACACTGG GCGTTGGCGC CGATCAAGGA TGCGCCATCA |
| | CAGGTACAGT AAGGAGAACC GGCCGCACGA ACATGACCGG TACTGTGACC CGCAACCGCG GCTAGTTCCT ACGCGGTAGT |
| | NS1 gene of YF17D |
| | --------------------- |
| | F G K R E L |
| | ACTTTGGCAA GAGAGAGCTC |
| | TGAAACCGTT CTCTCTCGAG |

CV-TBEV Hypr with YFV/WNV chimeric signal (p45)

| | |
|---|---|
| | 5′ UTR |
| | ---------------------------------------------------------------------------------------- |
| 1 | AGTAAATCCT GTGTGCTAAT TGAGGTGCAT TGGTCTGCAA ATCGAGTTGC TAGGCAATAA ACACATTTGG ATTAATTTTA |
| | TCATTTAGGA CACACGATTA ACTCCACGTA ACCAGACGTT TAGCTCAACG ATCCGTTATT TGTGTAAACC TAATTAAAAT |
| | 5′ UTR |
| | --------------------- |
| | ATCGTTCGTT GAGCGATTAG |
| | TAGCAAGCAA CTCGCTAATC |
| | 5′ UTR |
| | ------------------- |
| | C protein YF17D |
| | ---------------------------------------------------------------------- |
| | M S G R K A Q G K T L G V N M V R R G V R |
| 101 | CAGAGAACTG ACCAGAACAT GTCTGGTCGT AAAGCTCAGG GAAAAACCCT GGGCGTCAAT ATGGTACGAC GAGGAGTTCG |
| | GTCTCTTGAC TGGTCTTGTA CAGACCAGCA TTTCGAGTCC CTTTTTGGGA CCCGCAGTTA TACCATGCTG CTCCTCAAGC |
| | C protein YF17D |
| | ------------------- |
| | S L S N K I K • |
| | CTCCTTGTCA AACAAAATAA |
| | GAGGAACAGT TTGTTTTATT |
| | C protein YF17D |
| | ---------------------------------------------------------------------------------------- |
| | • Q K T K Q I G N R P G P S R G V Q G F I F F F L F N |
| 201 | AACAAAAAAC AAAACAAATT GGAAACAGAC CTGGACCTTC AAGAGGTGTT CAAGGATTTA TCTTTTTCTT TTTGTTCAAC |
| | TTGTTTTTTG TTTTGTTTAA CCTTTGTCTG GACCTGGAAG TTCTCCACAA GTTCCTAAAT AGAAAAAGAA AAACAAGTTG |
| | C protein YF17D |
| | ------------------- |
| | I L T G K K I • |
| | ATTTTGACTG GAAAAAAGAT |
| | TAAAACTGAC CTTTTTTCTA |
| | C protein YF17D |
| | ---------------------------------------------------------------------------------------- |
| | • T A H L K R L W K M L D P R Q G L A V L R K V K R V V |
| 301 | CACAGCCCAC CTAAAGAGGT TGTGGAAAAT GCTGGACCCA AGACAAGGCT TGGCTGTTCT AAGGAAAGTC AAGAGAGTGG |
| | GTGTCGGGTG GATTTCTCCA ACACCTTTTA CGACCTGGGT TCTGTTCCGA ACCGACAAGA TTCCTTTCAG TTCTCTCACC |
| | C protein YF17D |
| | ----------------------- |
| | A S L M R G |
| | TGGCCAGTTT GATGAGAGGA |
| | ACCGGTCAAA CTACTCTCCT |
| | C protein YF17D WNV partial signal |
| | ----------------------- -------------------------- |
| | YF 17D partial signal |
| | ---------------------------------------- |
| | L S S R K R R S H D V L T V Q F L I L G M L A C V G A |
| 401 | TTGTCCTCAA GGAAACGCCG TTCCCATGAT GTTCTGACTG TGCAATTCCT AATTTTGGGC ATGCTGGCTT GTGTCGGAGC A |
| | AACAGGAGTT CCTTTGCGGC AAGGGTACTA CAAGACTGAC ACGTTAAGGA TTAAAACCCG TACGACCGAA CACAGCCTCG T |
| | Hypr prM protein |
| | -------------------- |
| | A T V R K E R • |
| | GCTACCGTG CGAAAAGAAC |
| | CGATGGCAC GCTTTTCTTG |
| | Hypr prM protein |
| | ---------------------------------------------------------------------------------------- |
| | • D G S T V I R A E G K D A A T Q V R V E N G T C V I |
| 501 | GCGACGGAAG CACCGTGATA AGGGCTGAGG GTAAGGATGC GGCTACGCAG GTGAGAGTAG AGAATGGCAC TTGCGTAATA |
| | CGCTGCCTTC GTGGCACTAT TCCCGACTCC CATTCCTACG CCGATGCGTC CACTCTCATC TCTTACCGTG AACGCATTAT |
| | Hypr prM protein |
| | --------------------- |
| | L A T D M G S • |
| | CTCGCGACTG ATATGGGATC |
| | GAGCGCTGAC TATACCCTAG |
| | Hypr prM protein |
| | ---------------------------------------------------------------------------------------- |
| | • W C D D S L S Y E C V T I D Q G E E P V D V D C F C R |
| 601 | CTGGTGTGAC GATAGCCTCA GTTATGAATG CGTAACAATA GACCAGGGCG AAGAACCTGT GGACGTTGAC TGTTTCTGTA |
| | GACCACACTG CTATCGGAGT CAATACTTAC GCATTGTTAT CTGGTCCCGC TTCTTGGACA CCTGCAACTG ACAAAGACAT |
| | Hypr prM protein |
| | --------------------- |
| | N V D G V Y |
| | GAAATGTGGA TGGCGTTTAT |
| | CTTTACACCT ACCGCAAATA |
| | Hypr prM protein |
| | ---------------------------------------------------------------------------------------- |
| | L E Y G R C G K Q E G S R T R R S V L I P S H A Q G E |
| 701 | CTGGAGTACG GCCGCTGTGG AAAACAGGAG GGCTCACGAA CTCGAAGATC TGTGCTGATT CCAAGTCACG CGCAAGGAGA |
| | GACCTCATGC CGGCGACACC TTTTGTCCTC CCGAGTGCTT GAGCTTCTAG ACACGACTAA GGTTCAGTGC GCGTTCCTCT |
| | Hypr prM protein |
| | --------------------- |
| | L T G R G H K • |
| | GTTGACCGGT AGAGGCCACA |
| | CAACTGGCCA TCTCCGGTGT |
| | Hypr prM protein |
| | ---------------------------------------------------------------------------------------- |
| | • W L E G D S L R T H L T R V E G W V W K N R L L A L |
| 801 | AGTGGCTTGA AGGGGACTCA TTGAGGACCC ACCTGACTAG GGTGGAGGGT TGGGTTTGGA AGAATCGGTT GCTCGCGCTC |
| | TCACCGAACT TCCCCTGAGT AACTCCTGGG TGGACTGATC CCACCTCCCA ACCCAAACCT TCTTAGCCAA CGAGCGCGAG |
| | Hypr prM protein |
| | --------------------- |
| | A M V T V V W • |
| | GCTATGGTCA CCGTCGTGTG |
| | CGATACCAGT GGCAGCACAC |
| | Hypr prM protein |
| | -------------------------------------------------------------------------------- |
| | Hypr E |
| | protein |
| | -------- |
| | • L T L H S V V T R V A V L V V L L C L A P V Y A S R C |
| 901 | GCTGACACTG GAGAGTGTCG TGACTCGGGT TGCTGTGTTG GTTGTCCTCC TCTGTTTGGC CCCAGTGTAC GCGTCCAGGT |
| | CGACTGTGAC CTCTCACAGC ACTGAGCCCA ACGACACAAC CAACAGGAGG AGACAAACCG GGGTCACATG CGCAGGTCCA |
| | Hypr E protein |
| | --------------------- |
| | T H L E N R |
| | GTACTCATTT GGAAAACAGA |
| | CATGAGTAAA CCTTTTGTCT |
| | Hypr E protein |
| | ---------------------------------------------------------------------------------------- |
| | D F V T G T Q G T T R V T L V L E L G G C V T I T A E |
| 1001 | GATTTTGTCA CCGGCACCCA GGGGACGACT CGGGTAACCC TGGTGCTTGA ACTGGGTGGT TGCGTTACTA TTACCGCTGA |
| | CTAAAACAGT GGCCGTGGGT CCCCTGCTGA GCCCATTGGG ACCACGAACT TGACCCACCA ACGCAATGAT AATGGCGACT |
| | Hypr E protein |
| | --------------------- |
| | G K P S M D V • |
| | GGGCAAACCC TCTATGGATG |
| | CCCGTTTGGG AGATACCTAC |
| | Hypr E protein |
| | ---------------------------------------------------------------------------------------- |
| | • W L D A I Y Q E N P A Q T R E Y C L H A K L S D T K |
| 1101 | TGTGGCTGGA TGCAATCTAT CAGGAGAATC CCGCACAAAC CAGGGAATAT TGCCTTCACG CAAAGCTGTC CGATACAAAG |
| | ACACCGACCT ACGTTAGATA GTCCTCTTAG GGCGTGTTTG GTCCCTTATA ACGGAAGTGC GTTTCGACAG GCTATGTTTC |
| | Hypr E protein |
| | --------------------- |
| | V A A R C P T • |
| | GTCGCGGCTA GGTGCCCAAC |
| | CAGCGCCGAT CCACGGGTTG |
| | Hypr E protein |
| | ---------------------------------------------------------------------------------------- |
| | • M G P A T L A E E H Q G G T V C K R D Q S D R G W G N |
| 1201 | AATGGGACCG GCCACCCTGG CGGAGGAACA TCAGGGAGGT ACAGTGTGCA AACGGGACCA GAGTGATAGA GGCTGGGGTA |
| | TTACCCTGGC CGGTGGGACC GCCTCCTTGT AGTCCCTCCA TGTCACACGT TTGCCCTGGT CTCACTATCT CCGACCCCAT |
| | Hypr E protein |
| | --------------------- |
| | H C G L F G |
| | ATCACTGCGG CCTGTTCGGC |
| | TAGTGACGCC GGACAAGCCG |
| | Hypr E protein |
| | ---------------------------------------------------------------------------------------- |
| | K G S I V A C V K A A C E A K K K A T G H V Y D A N K |
| 1301 | AAAGGAAGTA TTGTCGCTTG CGTCAAGGCA GCCTGTGAGG CCAAAAAGAA GGCTACTGGG CACGTCTATG ACGCCAACAA |
| | TTTCCTTCAT AACAGCGAAC GCAGTTCCGT CGGACACTCC GGTTTTTCTT CCGATGACCC GTGCAGATAC TGCGGTTGTT |
| | Hypr E protein |
| | --------------------- |
| | I V Y T V K V • |
| | GATCGTTTAT ACAGTGAAAG |
| | CTAGCAAATA TGTCACTTTC |
| | Hypr E protein |
| | ---------------------------------------------------------------------------------------- |
| | • E P H T G D Y V A A N E T H S G R K T A S F T V S S |
| 1401 | TGGAACCACA CACAGGGGAT TACGTGGCGG CCAACGAGAC TCATTCCGGT CGCAAAACGG CCAGCTTCAC CGTGTCATCC |
| | ACCTTGGTGT GTGTCCCCTA ATGCACCGCC GGTTGCTCTG AGTAAGGCCA GCGTTTTGCC GGTCGAAGTG GCACAGTAGG |
| | Hypr E protein |
| | --------------------- |
| | E K T I L T M • |
| | GAAAAGACCA TCCTCACTAT |
| | CTTTTCTGGT AGGAGTGATA |
| | Hypr E protein |
| | ---------------------------------------------------------------------------------------- |
| | • G E Y G D V S L L C R V A S G V D L A Q T V I L E L D |
| 1501 | GGGGGAGTAT GGCGACGTTT CTCTGCTCTG CCGGGTGGCT AGCGGAGTCG ACCTGGCCCA GACAGTCATC CTGGAACTGG |
| | CCCCCTCATA CCGCTGCAAA GAGACGAGAC GGCCCACCGA TCGCCTCAGC TGGACCGGGT CTGTCAGTAG GACCTTGACC |
| | Hypr E protein |
| | --------------------- |
| | K T V E H L |
| | ATAAAACAGT TGAGCATCTG |
| | TATTTTGTCA ACTCGTAGAC |
| | Hypr E protein |
| | ---------------------------------------------------------------------------------------- |
| | P T A W Q V H R D W F N D L A L P W K H E G A R N W N |
| 1601 | CCTACCGCTT GGCAGGTGCA CAGGGATTGG TTTAACGACC TTGCCCTGCC ATGGAAACAT GAAGGAGCGA GAAACTGGAA |
| | GGATGGCGAA CCGTCCACGT GTCCCTAACC AAATTGCTGG AACGGGACGG TACCTTTGTA CTTCCTCGCT CTTTGACCTT |
| | Hypr E protein |
| | --------------------- |
| | N A E R L V E • |
| | TAATGCAGAG CGACTCGTAG |
| | ATTACGTCTC GCTGAGCATC |
| | Hypr E protein |
| | ---------------------------------------------------------------------------------------- |
| | • F G A P H A V K M D V Y N L G D Q T G V L L K A L A |
| 1701 | AATTCGGTGC CCCTCATGCC GTGAAGATGG ACGTCTACAA TCTGGGTGAT CAGACCGGCG TTCTCCTTAA AGCTCTCGCT |
| | TTAAGCCACG GGGAGTACGG CACTTCTACC TGCAGATGTT AGACCCACTA GTCTGGCCGC AAGAGGAATT TCGAGAGCGA |
| | Hypr E protein |
| | --------------------- |
| | G V P V A H I • |
| | GGCGTACCAG TTGCCCACAT |
| | CCGCATGGTC AACGGGTGTA |
| | Hypr E protein |
| | ---------------------------------------------------------------------------------------- |
| | • E G T K Y H L K S G H V T C E V G L E K L K M K G L T |
| 1801 | CGAAGGAACG AAGTACCACC TGAAGTCAGG CCATGTAACT TGCGAGGTGG GCCTGGAGAA GTTGAAAATG AAAGGTCTTA |
| | GCTTCCTTGC TTCATGGTGG ACTTCAGTCC GGTACATTGA ACGCTCCACC CGGACCTCTT CAACTTTTAC TTTCCAGAAT |
| | Hypr E protein |
| | --------------------- |
| | Y T M C D K |
| | CGTACACAAT GTGTGACAAG |
| | GCATGTGTTA CACACTGTTC |
| | Hypr E protein |
| | ---------------------------------------------------------------------------------------- |
| | T K F T W K R A P T D S G H D T V V M E V T F S G T K |
| 1901 | ACCAAGTTCA CATGGAAGAG GGCCCCCACA GATAGCGGCC ACGATACTGT GGTGATGGAG GTGACCTTTT CTGGAACAAA |
| | TGGTTCAAGT GTACCTTCTC CCGGGGGTGT CTATCGCCGG TGCTATGACA CCACTACCTC CACTGGAAAA GACCTTGTTT |
| | Hypr E protein |
| | --------------------- |
| | P C R I P V R • |
| | ACCCTGCAGA ATACCCGTGC |
| | TGGGACGTCT TATGGGCACG |
| | Hypr E protein |
| | ---------------------------------------------------------------------------------------- |
| | • A V A H G S P D V N V A M L I T P N P T I E N N G G |
| 2001 | GGGCTGTAGC TCACGGATCT CCCGATGTCA ATGTTGCTAT GCTGATTACA CCTAACCCTA CCATCGAGAA TAACGGTGGT |
| | CCCGACATCG AGTGCCTAGA GGGCTACAGT TACAACGATA CGACTAATGT GGATTGGGAT GGTAGCTCTT ATTGCCACCA |
| | Hypr E protein |
| | --------------------- |
| | G F I E M Q L • |
| | GGTTTTATTG AGATGCAGCT |
| | CCAAAATAAC TCTACGTCGA |
| | Hypr E protein |
| | ---------------------------------------------------------------------------------------- |
| | • P P G D N I I Y V G E L S Y Q W F Q K G S S I G R V F |
| 2101 | TCCGCCAGGC GATAACATCA TCTACGTGGG CGAACTCTCT TACCAGTGGT TTCAGAAAGG GAGTTCAATT GGGCGGGTCT |
| | AGGCGGTCCG CTATTGTAGT AGATGCACCC GCTTGAGAGA ATGGTCACCA AAGTCTTTCC CTCAAGTTAA CCCGCCCAGA |
| | Hypr E protein |
| | --------------------- |
| | Q K T K K G |
| | TCCAAAAAAC GAAGAAGGGA |
| | AGGTTTTTTG CTTCTTCCCT |
| | Hypr E protein |
| | ---------------------------------------------------------------------------------------- |
| | I E R L T V I G E H A W D F G S A G G F L S S I G K A |
| 2201 | ATCGAACGAT TGACGGTTAT CGGCGAGCAC GCATGGGATT TTGGTTCCGC AGGGGGATTC CTGTCTTCTA TTGGTAAGGC |
| | TAGCTTGCTA ACTGCCAATA GCCGCTCGTG CGTACCCTAA AACCAAGGCG TCCCCCTAAG GACAGAAGAT AACCATTCCG |
| | Hypr E protein |
| | --------------------- |
| | L H T V L G G • |
| | ACTGCATACC GTGCTGGGGG |
| | TGACGTATGG CACGACCCCC |
| | Hypr E protein |
| | ---------------------------------------------------------------------------------------- |
| | • A F N S I F G G V G F L P K L L L G V A L A W L G L |
| 2301 | GCGCATTCAA TTCTATTTTC GGGGGCGTGG GGTTCCTGCC TAAACTCCTG CTGGGAGTAG CCCTGGCCTG GTTGGGACTG |
| | CGCGTAAGTT AAGATAAAAG CCCCCGCACC CCAAGGACGG ATTTGAGGAC GACCCTCATC GGGACCGGAC CAACCCTGAC |
| | Hypr E protein |
| | --------------------- |
| | N M R N P T M • |
| | AATATGCGGA ATCCGACGAT |
| | TTATACGCCT TAGGCTGCTA |
| | Hypr E protein |
| | ------------------------------------------------------------------- |
| | NS1 gene of YF17D |
| | --------------------- |
| | • S M S F L L A G V L V L A M T L G V G A D Q G C A I N |
| 2401 | GTCCATGTCA TTCCTCTTGG CCGGCGTGCT TGTACTGGCC ATGACACTGG GCGTTGGCGC CGATCAAGGA TGCGCCATCA |
| | CAGGTACAGT AAGGAGAACC GGCCGCACGA ACATGACCGG TACTGTGACC CGCAACCGCG GCTAGTTCCT ACGCGGTAGT |
| | NS1 gene of YF17D |
| | --------------------- |
| | F G K R E L |
| | ACTTTGGCAA GAGAGAGCTC |
| | TGAAACCGTT CTCTCTCGAG |

CV-LGTV E5 with YFV/TBEV chimeric signal (p43)

| | |
|---|---|
| | 5′ UTR |
| | ---------------------------------------------------------------------------------------- |
| 1 | AGTAAATCCT GTGTGCTAAT TGAGGTGCAT TGGTCTGCAA ATCGAGTTGC TAGGCAATAA ACACATTTGG ATTAATTTTA |
| | TCATTTAGGA CACACGATTA ACTCCACGTA ACCAGACGTT TAGCTCAACG ATCCGTTATT TGTGTAAACC TAATTAAAAT |
| | 5′ UTR |
| | --------------------- |
| | ATCGTTCGTT GAGCGATTAG |
| | TAGCAAGCAA CTCGCTAATC |
| | 5′ UTR |
| | ------------------- |
| | C protein YF17D |
| | --------------------------------------------------------------------- |
| | M S G R K A Q G K T L G V N M V R R G V R |
| 101 | CAGAGAACTG ACCAGAACAT GTCTGGTCGT AAAGCTCAGG GAAAAACCCT GGGCGTCAAT ATGGTACGAC GAGGAGTTCG |
| | GTCTCTTGAC TGGTCTTGTA CAGACCAGCA TTTCGAGTCC CTTTTTGGGA CCCGCAGTTA TACCATGCTG CTCCTCAAGC |
| | C protein YF17D |
| | --------------------- |
| | S L S N K I K • |
| | CTCCTTGTCA AACAAAATAA |
| | GAGGAACAGT TTGTTTTATT |
| | C protein YF17D |
| | ---------------------------------------------------------------------------------------- |
| | • Q K T K Q I G N R P G P S R G V Q G F I F F F L F N |
| 201 | AACAAAAAAC AAAACAAATT GGAAACAGAC CTGGACCTTC AAGAGGTGTT CAAGGATTTA TCTTTTTCTT TTTGTTCAAC |
| | TTGTTTTTTG TTTTGTTTAA CCTTTGTCTG GACCTGGAAG TTCTCCACAA GTTCCTAAAT AGAAAAAGAA AAACAAGTTG |
| | C protein YF17D |
| | --------------------- |
| | I L T G K K I • |
| | ATTTTGACTG GAAAAAAGAT |
| | TAAAACTGAC CTTTTTTCTA |
| | C protein YF17D |
| | ---------------------------------------------------------------------------------------- |
| | • T A H L K R L W K M L D P R Q G L A V L R K V K R V V |
| 301 | CACAGCCCAC CTAAAGAGGT TGTGGAAAAT GCTGGACCCA AGACAAGGCT TGGCTGTTCT AAGGAAAGTC AAGAGAGTGG |
| | GTGTCGGGTG GATTTCTCCA ACACCTTTTA CGACCTGGGT TCTGTTCCGA ACCGACAAGA TTCCTTTCAG TTCTCTCACC |
| | C protein YF17D |
| | --------------------- |
| | A S L M R G |
| | TGGCCAGTTT GATGAGAGGA |
| | ACCGGTCAAA CTACTCTCCT |
| | C protein YF17D TBEV partial signal |
| | ----------------------- -------------------------- |
| | YF 17D partial signal |
| | ---------------------------------------- |
| | L S S R K R R S H D V L T V Q F L I L G M L G M T I A |
| 401 | TTGTCCTCAA GGAAACGCCG TTCCCATGAT GTTCTGACTG TGCAATTCCT AATTTTGGGC ATGCTGGGGA TGACGATCGC A |
| | AACAGGAGTT CCTTTGCGGC AAGGGTACTA CAAGACTGAC ACGTTAAGGA TTAAAACCCG TACGACCCCT ACTGCTAGCG T |
| | prM protein Langat E5 |
| | -------------------- |
| | A T V R R E R • |
| | GCTACTGTG CGAAGGGAGA |
| | CGATGACAC GCTTCCCTCT |
| | prM protein Langat E5 |
| | ---------------------------------------------------------------------------------------- |
| | • D G S M V I R A E G R D A A T Q V R V E N G T C V I |
| 501 | GAGACGGCTC TATGGTGATC AGAGCCGAAG GTAGGGACGC TGCGACCCAG GTGAGGGTCG AAAATGGCAC CTGTGTTATT |
| | CTCTGCCGAG ATACCACTAG TCTCGGCTTC CATCCCTGCG ACGCTGGGTC CACTCCCAGC TTTTACCGTG GACACAATAA |
| | prM protein Langat E5 |
| | --------------------- |
| | L A T D M G S • |
| | CTGGCGACCG ACATGGGCTC |
| | GACCGCTGGC TGTACCCGAG |
| | prM protein Langat E5 |
| | ---------------------------------------------------------------------------------------- |
| | • W C D D S L A Y E C V T I D Q G E E P V D V D C F C R |
| 601 | CTGGTGTGAT GATTCTCTGG CTTATGAATG TGTTACTATT GATCAGGGTG AAGAGCCTGT GGACGTGGAC TGTTTCTGTA |
| | GACCACACTA CTAAGAGACC GAATACTTAC ACAATGATAA CTAGTCCCAC TTCTCGGACA CCTGCACCTG ACAAAGACAT |
| | prM protein Langat E5 |
| | --------------------- |
| | G V E K V T |
| | GAGGCGTCGA GAAAGTGACC |
| | CTCCGGAGCT CTTTCACTGG |
| | prM protein Langat E5 |
| | ---------------------------------------------------------------------------------------- |
| | L E Y G R C G R R E G S R S R R S V L I P S H A Q R D |
| 701 | CTGGAATATG GACGATGTGG CCGGCGAGAA GGCTCCAGGA GTCGGAGATC CGTGTTGATC CCTTCACATG CGCAGCGCGA |
| | GACCTTATAC CTGCTACACC GGCCGCTCTT CCGAGGTCCT CAGCCTCTAG GCACAACTAG GGAAGTGTAC GCGTCGCGCT |
| | prM protein Langat E5 |
| | --------------------- |
| | L T G R G H Q • |
| | TCTGACAGGG AGGGGTCACC |
| | AGACTGTCCC TCCCCAGTGG |
| | prM protein Langat E5 |
| | ---------------------------------------------------------------------------------------- |
| | • W L E G E A V K A H L T R V E G W V W K N K L F T L |
| 801 | AGTGGCTCGA AGGCGAAGCA GTCAAGGCCC ATCTGACTCG CGTTGAAGGC TGGGTGTGGA AAAACAAACT CTTTACCCTT |
| | TCACCGAGCT TCCGCTTCGT CAGTTCCGGG TAGACTGAGC GCAACTTCCG ACCCACACCT TTTTGTTTGA GAAATGGGAA |
| | prM protein Langat E5 |
| | --------------------- |
| | S L V M V A W • |
| | AGCCTGGTGA TGGTCGCGTG |
| | TCGGACCACT ACCAGCGCAC |
| | prM protein Langat E5 |
| | -------------------------------------------------------------------------------- |
| | E protein |
| | Langat E5 |
| | -------- |
| | • L M V D G L L P R I L I V V V A L A L A P A Y A S R C |
| 901 | GCTGATGGTA GACGGACTCC TTCCCCGCAT TCTCATTGTT GTGGTGGCTC TCGCGCTCGC CCCTGCATAC GCGTCCAGGT |
| | CGACTACCAT CTGCCTGAGG AAGGGGCGTA AGAGTAACAA CACCACCGAG AGCGCGAGCG GGGACGTATG CGCAGGTCCA |
| | E protein Langat E5 |
| | --------------------- |
| | T H L E N R |
| | GTACGCACCT CGAAAATCGA |
| | CATGCGTGGA GCTTTTAGCT |
| | E protein Langat E5 |
| | ---------------------------------------------------------------------------------------- |
| | D F V T G V Q G T T R L T L V L E L G G C V T V T A D |
| 1001 | GATTTCGTCA CAGGCGTCCA AGGTACTACC CGGCTCACCC TCGTGCTGGA GCTGGGAGGC TGTGTCACTG TTACAGCCGA |
| | CTAAAGCAGT GTCCGCAGGT TCCATGATGG GCCGAGTGGG AGCACGACCT CGACCCTCCG ACACAGTGAC AATGTCGGCT |
| | E protein Langat E5 |
| | --------------------- |
| | G K P S L D V • |
| | CGGAAAACCT AGTCTGGATG |
| | GCCTTTTGGA TCAGACCTAC |
| | E protein Langat E5 |
| | ---------------------------------------------------------------------------------------- |
| | • W L D S I Y Q E S P A Q T R E Y C L H A K L T G T K |
| 1101 | TGTGGCTGGA CTCCATCTAT CAGGAGAGCC CGGCACAGAC CAGGGAGTAC TGCCTCCACG CTAAGCTGAC TGGGACAAAG |
| | ACACCGACCT GAGGTAGATA GTCCTCTCGG GCCGTGTCTG GTCCCTCATG ACGGAGGTGC GATTCGACTG ACCCTGTTTC |
| | E protein Langat E5 |
| | --------------------- |
| | V A A R C P T • |
| | GTAGCCGCAA GATGTCCCAC |
| | CATCGGCGTT CTACAGGGTG |
| | E protein Langat E5 |
| | ---------------------------------------------------------------------------------------- |
| | • M G P A T L P E E H Q S G T V C K R D Q S D R G W G N |
| 1201 | AATGGGGCCT GCCACCTTGC CCGAGGAACA CCAATCCGGT ACGGTATGCA AGCGAGATCA GTCTGATCGC GGATGGGGGA |
| | TTACCCCGGA CGGTGGAACG GGCTCCTTGT GGTTAGGCCA TGCCATACGT TCGCTCTAGT CAGACTAGCG CCTACCCCCT |
| | E protein Langat E5 |
| | --------------------- |
| | H C G L F G |
| | ATCATTGCGG CCTCTTCGGT |
| | TAGTAACGCC GGAGAAGCCA |
| | E protein Langat E5 |
| | ---------------------------------------------------------------------------------------- |
| | K G S I V T C V K V T C E D K K K A T G H V Y D V N K |
| 1301 | AAAGGCAGCA TTGTCACTTG CGTGAAGGTG ACATGCGAGG ACAAGAAGAA GGCCACAGGT CATGTATATG ATGTGAACAA |
| | TTTCCGTCGT AACAGTGAAC GCACTTCCAC TGTACGCTCC TGTTCTTCTT CCGGTGTCCA GTACATATAC TACACTTGTT |
| | E protein Langat E5 |
| | --------------------- |
| | I T Y T I K V • |
| | AATCACATAT ACCATTAAGG |
| | TTAGTGTATA TGGTAATTCC |
| | E protein Langat E5 |
| | ---------------------------------------------------------------------------------------- |
| | • E P H T G E F V A A N E T H S G R K S A S F T V S S |
| 1401 | TAGAACCACA TACAGGGGAA TTCGTGGCAG CAAACGAGAC TCATAGCGGA CGAAAGTCCG CCTCCTTCAC CGTCTCCTCC |
| | ATCTTGGTGT ATGTCCCCTT AAGCACCGTC GTTTGCTCTG AGTATCGCCT GCTTTCAGGC GGAGGAAGTG GCAGAGGAGG |
| | E protein Langat E5 |
| | --------------------- |
| | E K T I L T L • |
| | GAGAAAACAA TCCTGACCCT |
| | CTCTTTTGTT AGGACTGGGA |
| | E protein Langat E5 |
| | ---------------------------------------------------------------------------------------- |
| | • G D Y G D V S L L C R V A S G V D L A Q T V V L A L D |
| 1501 | CGGAGACTAC GGCGACGTAT CTTTGCTGTG CAGGGTGGCC AGCGGCGTGG ACCTTGCTCA GACAGTCGTG TTGGCCCTGG |
| | GCCTCTGATG CCGCTGCATA GAAACGACAC GTCCCACCGG TCGCCGCACC TGGAACGAGT CTGTCAGCAC AACCGGGACC |
| | E protein Langat E5 |
| | --------------------- |
| | K T H E H L |
| | ACAAGACACA TGAGCACTTG |
| | TGTTCTGTGT ACTCGTGAAC |
| | E protein Langat E5 |
| | ---------------------------------------------------------------------------------------- |
| | P T A W Q V H R D W F N D L A L P W K H D G A E A W N |
| 1601 | CCAACAGCCT GGCAGGTGCA CAGGGACTGG TTTAACGACC TGGCGCTCCC GTGGAAACAT GACGGCGCTG AAGCATGGAA |
| | GGTTGTCGGA CCGTCCACGT GTCCCTGACC AAATTGCTGG ACCGCGAGGG CACCTTTGTA CTGCCGCGAC TTCGTACCTT |
| | E protein Langat E5 |
| | --------------------- |
| | E A G R L V E • |
| | TGAGGCAGGG AGACTGGTGG |
| | ACTCCGTCCC TCTGACCACC |
| | E protein Langat E5 |
| | ---------------------------------------------------------------------------------------- |
| | • F G T P H A V K M D V F N L G D Q T G V L L K S L A |
| 1701 | AATTTGGAAC CCCACACGCC GTAAAGATGG ACGTTTTCAA TCTTGGTGAC CAGACAGGGG TGCTCCTGAA ATCACTGGCG |
| | TTAAACCTTG GGGTGTGCGG CATTTCTACC TGCAAAAGTT AGAACCACTG GTCTGTCCCC ACGAGGACTT TAGTGACCGC |
| | E protein Langat E5 |
| | --------------------- |
| | G V P V A S I • |
| | GGCGTGCCTG TAGCCAGCAT |
| | CCGCACGGAC ATCGGTCGTA |
| | E protein Langat E5 |
| | ---------------------------------------------------------------------------------------- |
| | • E G T K Y H L K S G H V T C E V G L S K L K M K G L T |
| 1801 | CGAGGGCACA AAGTATCACC TGAAGTCTGG GCATGTAACC TGCGAAGTGG GCCTGGAAAA GCTGAAGATG AAAGGACTTA |
| | GCTCCCGTGT TTCATAGTGG ACTTCAGACC CGTACATTGG ACGCTTCACC CGGACCTTTT CGACTTCTAC TTTCCTGAAT |
| | E protein Langat E5 |
| | --------------------- |
| | Y T V C D K |
| | CGTACACTGT TTGTGATAAG |
| | GCATGTGACA AACACTATTC |
| | E protein Langat E5 |
| | ---------------------------------------------------------------------------------------- |
| | T K F T W K R A P T D S G H D T V V M E V G F S G T R |
| 1901 | ACCAAGTTTA CATGGAAGCG AGCCCCAACG GATTCCGGCC ATGATACCGT CGTGATGGAG GTTGGTTTCT CCGGCACCAG |
| | TGGTTCAAAT GTACCTTCGC TCGGGGTTGC CTAAGGCCGG TACTATGGCA GCACTACCTC CAACCAAAGA GGCCGTGGTC |
| | E protein Langat E5 |
| | --------------------- |
| | P C R I P V R • |
| | ACCATGTAGA ATACCAGTGA |
| | TGGTACATCT TATGGTCACT |
| | E protein Langat E5 |
| | ---------------------------------------------------------------------------------------- |
| | • A V A H G V P E V N V A M L I T P N P T N E N N G G |
| 2001 | GAGCTGTCGC CCACGGTGTA CCCGAGGTAA ACGTGGCCAT GCTGATTACA CCGAATCCCA CTATGGAGAA CAATGGCGGA |
| | CTCGACAGCG GGTGCCACAT GGGCTCCATT TGCACCGGTA CGACTAATGT GGCTTAGGGT GATACCTCTT GTTACCGCCT |
| | E protein Langat E5 |
| | --------------------- |
| | G F I E M Q L • |
| | GGGTTCATCG AAATGCAGCT |
| | CCCAAGTAGC TTTACGTCGA |
| | E protein Langat E5 |
| | ---------------------------------------------------------------------------------------- |
| | • P P G D N I I Y V G D L D H Q W F Q K G S S I G R V L |
| 2101 | GCCGCCTGGA GACAACATCA TTTATGTCGG CGACCTCGAT CATCAATGGT TCCAGAAAGG GTCTTCCATC GGCCGCGTCC |
| | CGGCGGACCT CTGTTGTAGT AAATACAGCC GCTGGAGCTA GTAGTTACCA AGGTCTTTCC CAGAAGGTAG CCGGCGCAGG |
| | E protein Langat E5 |
| | --------------------- |
| | Q K T R K G |
| | TTCAGAAGAC ACGAAAAGGC |
| | AAGTCTTCTG TGCTTTTCCG |
| | E protein Langat E5 |
| | ---------------------------------------------------------------------------------------- |
| | I E R L T V L G E H A W D F G S V G G V M T S I G R A |
| 2201 | ATTGAAAGAC TTACAGTCCT GGGCGAACAT GCCTGGGACT TCGGGTCAGT TGGCGGGGTA ATGACAAGCA TAGGCAGAGC |
| | TAACTTTCTG AATGTCAGGA CCCGCTTGTA CGGACCCTGA AGCCCAGTCA ACCGCCCCAT TACTGTTCGT ATCCGTCTCG |
| | E protein Langat E5 |
| | --------------------- |
| | M H T V L G G • |
| | TATGCACACC GTTCTCGGTG |
| | ATACGTGTGG CAAGAGCCAC |
| | E protein Langat E5 |
| | ---------------------------------------------------------------------------------------- |
| | • A F N T L L G G V G F L P K I L L G V A M A W L G L |
| 2301 | GGGCATTTAA TACTCTGTTG GGTGGCGTGG GTTTTCTTCC GAAAATCCTG CTCGGTGTCG CAATGGCCTG GCTTGGACTG |
| | CCCGTAAATT ATGAGACAAC CCACCGCACC CAAAAGAAGG CTTTTAGGAC GAGCCACAGC GTTACCGGAC CGAACCTGAC |
| | E protein Langat E5 |
| | --------------------- |
| | N M R N P T L • |
| | AATATGCGCA ATCCTACACT |
| | TTATACGCGT TAGGATGTGA |
| | E protein Langat E5 |
| | ------------------------------------------------------------------- |
| | NS1 gene of YF17D |
| | --------------------- |
| | • S M G F L L S G G L V L A M T L G V G A D Q G C A I N |
| 2401 | GAGTATGGGG TTTCTTCTGT CAGGAGGCCT GGTCCTGGCA ATGACTCTGG GAGTGGGCGC CGATCAAGGA TGCGCCATCA |
| | CTCATACCCC AAAGAAGACA GTCCTCCGGA CCAGGACCGT TACTGAGACC CTCACCCGCG GCTAGTTCCT ACGCGGTAGT |
| | NS1 gene of YF17D |
| | --------------------- |
| | F G K R E L |
| | ACTTTGGCAA GAGAGAGCTC |
| | TGAAACCGTT CTCTCTCGAG |

CV-TBEV Hypr with YFV/TBEV chimeric signal and dC2 deletion in C protein (p59)

| | |
|---|---|
| | 5′ UTR |
| | ---------------------------------------------------------------------------------------- |
| 1 | AGTAAATCCT GTGTGCTAAT TGAGGTGCAT TGGTCTGCAA ATCGAGTTGC TAGGCAATAA ACACATTTGG ATTAATTTTA |
| | TCATTTAGGA CACACGATTA ACTCCACGTA ACCAGACGTT TAGCTCAACG ATCCGTTATT TGTGTAAACC TAATTAAAAT |
| | 5′ UTR |
| | --------------------- |
| | ATCGTTCGTT GAGCGATTAG |
| | TAGCAAGCAA CTCGCTAATC |
| | 5′ UTR |
| | ------------------- |
| | C protein |
| | --------------------------------------------------------------------- |
| | M S G R K A Q G K T L G V N M V R R G V R |
| 101 | CAGAGAACTG ACCAGAACAT GTCTGGTCGT AAAGCTCAGG GAAAAACCCT GGGCGTCAAT ATGGTACGAC GAGGAGTTCG |
| | GTCTCTTGAC TGGTCTTGTA CAGACCAGCA TTTCGAGTCC CTTTTTGGGA CCCGCAGTTA TACCATGCTG CTCCTCAAGC |
| | C protein |
| | --------------------- |
| | S L S N K I K • |
| | CTCCTTGTCA AACAAAATAA |
| | GAGGAACAGT TTGTTTTATT |
| | dC2 deletion (PSR) |
| | - |
| | C protein |
| | ---------------------------------------------------------------------------------------- |
| | • Q K T K Q I G N R P G G V Q G F I F F F L F N I L T |
| 201 | AACAAAAAAC AAAACAAATT GGAAACAGAC CTGGAGGTGT TCAAGGATTT ATCTTTTTCT TTTTGTTCAA CATTTTGACT |
| | TTGTTTTTTG TTTTGTTTAA CCTTTGTCTG GACCTCCACA AGTTCCTAAA TAGAAAAAGA AAAACAAGTT GTAAAACTGA |
| | C protein |
| | --------------------- |
| | G K K I T A H • |
| | GGAAAAAAGA TCACAGCCCA |
| | CCTTTTTTCT AGTGTCGGGT |
| | C protein |
| | ---------------------------------------------------------------------------------------- |
| | • L K R L W K M L D P R Q G L A V L R K V K R V V A S L |
| 301 | CCTAAAGAGG TTGTGGAAAA TGCTGGACCC AAGACAAGGC TTGGCTGTTC TAAGGAAAGT CAAGAGAGTG GTGGCCAGTT |
| | GGATTTCTCC AACACCTTTT ACGACCTGGG TTCTGTTCCG AACCGACAAG ATTCCTTTCA GTTCTCTCAC CACCGGTCAA |
| | C protein |
| | --------------------- |
| | M R G L S S |
| | TGATGAGAGG ATTGTCCTCA |
| | ACTACTCTCC TAACAGGAGT |
| | YE17D partial signal |
| | --------------------------------------- |
| | TBEV partial signal |
| | --------------------------- |
| | C protein Hypr prM |
| | protein |
| | ------------- --------- |
| | R K R R S H D V L T V Q F L I L G M L G M T I A A T V |
| 401 | AGGAAACGCC GTTCCCATGA TGTTCTGACT GTGCAATTCC TAATTTTGGG CATGCTGGGC ATGACAATCG CAGCTACGGT |
| | TCCTTTGCGG CAAGGGTACT ACAAGACTGA CACGTTAAGG ATTAAAACCC GTACGACCCG TACTGTTAGC GTCGATGCCA |
| | Hypr prM protein |
| | --------------------- |
| | R K E R D G S • |
| | TCGCAAGGAA AGAGACGGCA |
| | AGCGTTCCTT TCTCTGCCGT |
| | Hypr prM protein |
| | ---------------------------------------------------------------------------------------- |
| | • T V I R A E G K D A A T Q V R V E N G T C V I L A T |
| 501 | GTACGGTCAT ACGCGCGGAA GGTAAGGATG CCGCTACCCA AGTGAGAGTG GAAAATGGTA CCTGCGTCAT TCTGGCCACC |
| | CATGCCAGTA TGCGCGCCTT CCATTCCTAC GGCGATGGGT TCACTCTCAC CTTTTACCAT GGACGCAGTA AGACCGGTGG |
| | Hypr prM protein |
| | --------------------- |
| | D M G S W C D • |
| | GACATGGGCT CTTGGTGTGA |
| | CTGTACCCGA GAACCACACT |
| | Hypr prM protein |
| | ---------------------------------------------------------------------------------------- |
| | • D S L S Y E C V T I D Q G E E P V D V D C F C R N V D |
| 601 | TGATAGCCTT TCTTATGAGT GCGTAACCAT AGATCAAGGT GAGGAACCTG TTGACGTTGA TTGCTTCTGC CGAAACGTGG |
| | ACTATCGGAA AGAATACTCA CGCATTGGTA TCTAGTTCCA CTCCTTGGAC AACTGCAACT AACGAAGACG GCTTTGCACC |
| | Hypr prM protein |
| | --------------------- |
| | G V Y L E Y |
| | ATGGGGTGTA TCTCGAATAT |
| | TACCCCACAT AGAGCTTATA |
| | Hypr prM protein |
| | ---------------------------------------------------------------------------------------- |
| | G R C G K Q E G S R T R R S V L I P S H A Q G E L T G |
| 701 | GGACGGTGTG GTAAACAAGA AGGAAGCAGA ACCAGACGCT CAGTGCTTAT ACCCTCCCAC GCTCAAGGAG AGCTGACCGG |
| | CCTGCCACAC CATTTGTTCT TCGTTCGTCT TGGTCTGCGA GTCACGAATA TGGGAGGGTG CGAGTTCCTC TCGACTGGCC |
| | Hypr prM protein |
| | --------------------- |
| | R G H K W L E • |
| | ACGGGGACAT AAATGGTTGG |
| | TGCCCCTGTA TTTACCAACC |
| | Hypr prM protein |
| | ---------------------------------------------------------------------------------------- |
| | • G D S L R T H L T R V E G W V N K N R L L A L A M V |
| 801 | AGGGCGACTC ACTCCGAACA CATTTGACCC GCGTCGAGGG CTGGGTCTGG AAAAATCGGC TGTTGGCCCT CGCTATGGTG |
| | TCCCGCTGAG TGAGGCTTGT GTAAACTGGG CGCAGCTCCC GACCCAGACC TTTTTAGCCG ACAACCGGGA GCGATACCAC |
| | Hypr prM protein |
| | --------------------- |
| | T V V W L T L • |
| | ACAGTCGTTT GGCTCACGCT |
| | TGTCAGCAAA CCGAGTGCGA |
| | Hypr E |
| | protein |
| | ------------------ |
| | Hypr prM protein |
| | ---------------------------------------------------------------------- |
| | • E S V V T R V A V L V V L L C L A P V Y A S R C T H L |
| 901 | GGAGTCTGTG GTTACTCGCG TGGCAGTGCT GGTGGTGCTC CTCTGTCTTG CCCCTGTCTA CGCGTCCAGG TGTACTCATT |
| | CCTCAGACAC CAATGAGCGC ACCGTCACGA CCACCACCAG GAGACAGAAC GGGGACAGAT GCGCAGGTCC ACATGAGTAA |
| | Hypr E protein |
| | --------------------- |
| | E N R D F V |
| | TGGAAAACAG AGATTTTGTC |
| | ACCTTTTGTC TCTAAAACAG |
| | Hypr E protein |
| | ---------------------------------------------------------------------------------------- |
| | T G T Q G T T K V T L V L E L G G C V T I T A E G K P |
| 1001 | ACCGGCACCC AGGGGACGAC TCGGGTAACC CTGGTGCTTG AACTGGGTGG TTGCGTTACT ATTACCGCTG AGGGCAAACC |
| | TGGCCTGGG TCCCCTGCTG AGCCCATTGG GACCACGAAC TTGACCCACC AACGCAATGA TAATGGCGAC TCCCGTTTGG |
| | Hypr E protein |
| | --------------------- |
| | S M D V W L D • |
| | CTCTATGGAT GTGTGGCTGG |
| | GAGATACCTA CACACCGACC |
| | Hypr E protein |
| | ---------------------------------------------------------------------------------------- |
| | • A I Y Q E N P A Q T R E Y C L H A K L S D T K V A A |
| 1101 | ATGCAATCTA TCAGGAGAAT CCCGCACAAA CCAGGGAATA TTGCCTTCAC GCAAAGCTGT CCGATACAAA GGTCGCGGCT |
| | TACGTTAGAT AGTCCTCTTA GGGCGTGTTT GGTCCCTTAT AACGGAAGTG CGTTTCGACA GGCTATGTTT CCAGCGCCGA |
| | Hypr E protein |
| | --------------------- |
| | R C P T M G P • |
| | AGGTGCCCAA CAATGGGACC |
| | TCCACGGGTT GTTACCCTGG |
| | Hypr E protein |
| | ---------------------------------------------------------------------------------------- |
| | • A T L A E E H Q G G T V C K R D Q S D R G W G N H C G |
| 1201 | GGCCACCCTG GCGGAGGAAC ATCAGGGAGG TACAGTGTGC AAACGGGACC AGAGTGATAG AGGCTGGGGT AATCACTGCG |
| | CCGGTGGGAC CGCCTCCTTG TAGTCCCTCC ATGTCACACG TTTGCCCTGG TCTCACTATC TCCGACCCCA TTAGTGACGC |
| | Hypr E protein |
| | --------------------- |
| | L F G K G S |
| | GCCTGTTCGG CAAAGGAAGT |
| | CGGACAAGCC GTTTCCTTCA |
| | Hypr E protein |
| | ---------------------------------------------------------------------------------------- |
| | I V A C V K A A C E A K K K A T G H V Y D A N K I V Y |
| 1301 | ATTGTCGCTT GCGTCAAGGC AGCCTGTGAG GCCAAAAAGA AGGCTACTGG GCACGTCTAT GACGCCAACA AGATCGTTTA |
| | TAACAGCGAA CGCAGTTCCG TCGGACACTC CGGTTTTTCT TCCGATGACC CGTGCAGATA CTGCGGTTGT TCTAGCAAAT |
| | Hypr E protein |
| | --------------------- |
| | T V K V E P H • |
| | TACAGTGAAA GTGGAACCAC |
| | ATGTCACTTT CACCTTGGTG |
| | Hypr E protein |
| | ---------------------------------------------------------------------------------------- |
| | • T G D Y V A A N E T H S G R K T A S F T V S S E K T |
| 1401 | ACACAGGGGA TTACGTGGCG GCCAACGAGA CTCATTCCGG TCGCAAAACG GCCAGCTTCA CCGTGTCATC CGAAAAGACC |
| | TGTGTCCCCT AATGCACCGC CGGTTGCTCT GAGTAAGGCC AGCGTTTTGC CGGTCGAAGT GGCACAGTAG GCTTTTCTGG |
| | Hypr E protein |
| | --------------------- |
| | I L T M G E Y • |
| | ATCCTCACTA TGGGGGAGTA |
| | TAGGAGTGAT ACCCCCTCAT |
| | Hypr E protein |
| | ---------------------------------------------------------------------------------------- |
| | • G D V S L L C R V A S G V D L A Q T V I L E L D K T V |
| 1501 | TGGCGACGTT TCTCTGCTCT GCCGGGTGGC TAGCGGAGTC GACCTGGCCC AGACAGTCAT CCTGGAACTG GATAAAACAG |
| | ACCGCTGCAA AGAGACGAGA CGGCCCACCG ATCGCCTCAG CTGGACCGGG TCTGTCAGTA GGACCTTGAC CTATTTTGTC |
| | Hypr E protein |
| | --------------------- |
| | E H L P T A |
| | TTGAGCATCT GCCTACCGCT |
| | AACTCGTAGA CGGATGGCGA |
| | Hypr E protein |
| | ---------------------------------------------------------------------------------------- |
| | W Q V H R D W F N D L A L P W K H E G A R N W N N A E |
| 1601 | TGGCAGGTGC ACAGGGATTG GTTTAACGAC CTTGCCCTGC CATGGAAACA TGAAGGAGCG AGAAACTGGA ATAATGCAGA |
| | ACCGTCCACG TGTCCCTAAC CAAATTGCTG GAACGGGACG GTACCTTTGT ACTTCCTCGC TCTTTGACCT TATTACGTCT |
| | Hypr E protein |
| | --------------------- |
| | R L V E F G A • |
| | GCGACTCGTA GAATTCGGTG |
| | CGCTGAGCAT CTTAAGCCAC |
| | Hypr E protein |
| | ---------------------------------------------------------------------------------------- |
| | • P H A V K M D V Y N L G D Q T G V L L K A L A G V P |
| 1701 | CCCCCTCATGC CGTGAAGATG GACGTCTACA ATCTGGGTGA TCAGACCGGC GTTCTCCTTA AAGCTCTCGC TGGCGTACCA |
| | GGGGAGTACG GCACTTCTAC CTGCAGATGT TAGACCCACT AGTCTGGCCG CAAGAGGAAT TTCGAGAGCG ACCGCATGGT |
| | Hypr E protein |
| | --------------------- |
| | V A H I E G T • |
| | GTTGCCCACA TCGAAGGAAC |
| | CAACGGGTGT AGCTTCCTTG |
| | Hypr E protein |
| | ---------------------------------------------------------------------------------------- |
| | • K Y H L K S G H V T C E V G L E K L K M K G L T Y T M |
| 1801 | CAAGTACCAC CTGAAGTCAG GCCATGTAAC TTGCGAGGTG GGCCTGGAGA AGTTGAAAT GAAAGGTCTT ACGTACACAA |
| | CTTCATGGTG GACTTCAGTC CGGTACATTG AACGCTCCAC CCGGACCTCT TCAACTTTTA CTTTCCAGAA TGCATGTGTT |
| | Hypr E protein |
| | --------------------- |
| | C D K T K F |
| | TGTGTGACAA GACCAAGTTC |
| | ACACACTGTT CTGGTTCAAG |
| | Hypr E protein |
| | ---------------------------------------------------------------------------------------- |
| | T W K R A P T D S G H D T V V M E V T F S G T K P C R |
| 1901 | ACATGGAAGA GGGCCCCCAC AGATAGCGGC CACGATACTG TGGTGATGGA GGTGACCTTT TCTGGAACAA AACCCTGCAG |
| | TGTACCTTCT CCCGGGGGTG TCTATCGCCG GTGCTATGAC ACCACTACCT CCACTGGAAA AGACCTTGTT TTGGGACGTC |
| | Hypr E protein |
| | --------------------- |
| | I P V R A V A • |
| | AATACCCGTG CGGGCTGTAG |
| | TTATGGGCAC GCCCGACATC |
| | Hypr E protein |
| | ---------------------------------------------------------------------------------------- |
| | • H G S P D V N V A M L I T P N P T I E N N G G G F I |
| 2001 | CTCACGGATC TCCCGATGTC AATGTTGCTA TGCTGATTAC ACCTAACCCT ACCATCGAGA ATAACGGTGG TGGTTTTATT |
| | GAGTGCCTAG AGGGCTACAG TTACAACGAT ACGACTAATG TGGATTGGGA TGGTAGCTCT TATTGCCACC ACCAAATAA |
| | Hypr E protein |
| | --------------------- |
| | E M Q L P P G • |
| | GAGATGCAGC TTCCGCCAGG |
| | CTCTACGTCG AAGGCGGTCC |
| | Hypr E protein |
| | ---------------------------------------------------------------------------------------- |
| | • D N I I Y V G E L S Y Q W F Q K G S S I G R V F Q K T |
| 2101 | CGATAACATC ATCTACGTGG GCGAACTCTC TTACCAGTGG TTTCAGAAAG GGAGTTCAAT TGGGCGGGTC TTCCAAAAAA |
| | GCTATTGTAG TAGATGCACC CGCTTGAGAG AATGGTCACC AAAGTCTTTC CCTCAAGTTA ACCCGCCCAG AAGGTTTTTT |
| | Hypr E protein |
| | --------------------- |
| | K K G I E R |
| | CGAAGAAGGG AATCGAACGA |
| | GCTTCTTCCC TTAGCTTGCT |
| | Hypr E protein |
| | ---------------------------------------------------------------------------------------- |
| | L T V I G E H A W D F G S A G G F L S S I G K A L H T |
| 2201 | TTGACGGTTA TCGGCGAGCA CGCATGGCAT TTTGGTTCCG CAGGGGGATT CCTGTCTTCT ATTGGTAAGG CACTGCATAC |
| | AACTGCCAAT AGCCGCTCGT GCGTACCCTA AAACCAAGGC GTCCCCCTAA GGACAGAAGA TAACCATTCC GTGACGTATG |
| | Hypr E protein |
| | --------------------- |
| | V L G G A F N • |
| | CGTGCTGGGG GGCGCATTCA |
| | GCACGACCCC CCGCGTAAGT |
| | Hypr E protein |
| | ---------------------------------------------------------------------------------------- |
| | • S I F G G V G F L P K L L L G V A L A W L G L N M R |
| 2301 | ATTCTATTTT CGGGGGCGTG GGGTTCCTGC CTAAACTCCT GCTGGGAGTA GCCCTGGCCT GGTTGGGACT GAATATGCGG |
| | TAAGATAAAA GCCCCCGCAC CCCAAGGACG GATTTGAGGA CGACCCTCAT CGGACCGGA CCAACCCTGA CTTATACGCC |
| | Hypr E protein |
| | --------------------- |
| | N P T M S M S • |
| | AATCCGACGA TGTCCATGTC |
| | TTAGGCTGCT ACAGGTACAG |
| | Hypr E protein |
| | -------------------------------------------------------- |
| | NS1 gene of YF17D |
| | ------------------------------- |
| | • F L L A G V L V L A M T L G V G A D Q G C A I N F G K |
| 2401 | ATTCCTCTTG GCCGGCGTGC TTGTACTGGC CATGACACTG GGCGTTGGCG CCGATCAAGG ATGCGCCATC AACTTTGGCA |
| | TAAGGAGAAC CGGCCGCACG AACATGACCG GTACTGTGAC CCGCAACCGC GGCTAGTTCC TACGCGGTAG TTGAAACCGT |
| | NS1 gene of YF17D |
| | ------------ |
| | R E L |
| | AGAGAGAGCT C |
| | TCTCTCTCGA G |

SEQUENCE APPENDIX 3
PIV-WN/TBEV Hypr with TBEV signal (p39)

| | |
|---|---|
| | 5′ UTR |
| | ---------------------------------------------------------------------------------------- |
| 1 | AGTAGTTCGC CTGTGTGAGC TGACAAACTT AGTAGTGTT GTGAGGATTA ACAACAATTA ACACAGTGCG AGCTGTTTCT |
| | TCATCAAGCG GACACACTCG ACTGTTTGAA TCATCACAAA CACTCCTAAT TGTTGTTAAT TGTGTCACGC TCGACAAAGA |
| | deleted C |
| | ---- |
| | 5′ UTR |
| | ----------------- |
| | M S • |
| | TAGCACGAAG ATCTCGATGT |
| | ATCGTGCTTC TAGAGCTACA |
| | WNV deleted C protein |
| | ---------------------------------------------------------------------------------------- |
| | • K K P G G P G K S R A V Y L L K R G M P R V L S L I |
| 101 | CTAAGAAACC AGGAGGGCCC GGCAAGAGCC GGGCTGTCTA TTTGCTAAAA CGCGGAATGC CCCGCGTGTT GTCCTTGATT |
| | GATTCTTTGG TCCTCCCGGG CCGTTCTCGG CCCGACAGAT AAACGATTTT GCGCCTTACG GGGCGCACAA CAGGAACTAA |
| | WNV deleted |
| | C protein |
| | --------------------- |
| | G L K R S S K • |
| | GGACTTAAGC GGAGCTCCAA |
| | CCTGAATTCG CCTCGAGGTT |
| | TSEV signal |
| | ----------------------------------------------------------------- |
| | deleted C prM Hypr |
| | -------------- --------- |
| | • Q K K R G G T D W M S W L L V I G M L G M T I A A T V |
| 201 | ACAAAAGAAA CGGGGGGGAA CAGACTGGAT GAGCTGGCTG CTCGTAATCG GCATGCTGGG CATGACAATC GCAGCTACGG |
| | TGTTTTCTTT GCCCCCCCTT GTCTGACCTA CTCGACCGAC GAGCATTAGC CGTACGACCC GTACTGTTAG CGTCGATGCC |
| | prM Hypr |
| | ---------------------- |
| | R K E R D G |
| | TTCGCAAGGA AAGAGACGGC |
| | AAGCGTTCCT TTCTCTGCCG |
| | prM Hypr |
| | -------------------------------------------------------------------------------------- |
| | S T V I R A E G K D A A T Q V R V E N G T C V I L A T |
| 301 | AGTACGGTCA TACGCGCGGA AGGTAAGGAT GCCGCTACCC AAGTGAGAGT GGAAAATGGT ACCTGCGTCA TTCTGGCCAC |
| | TCATGCCAGT ATGCGCGCCT TCCATTCCTA CGGCGATGGG TTCACTCTCA CCTTTTACCA TGGACGCAGT AAGACCGGTG |
| | prM Hypr |
| | --------------------- |
| | D M G S W C D • |
| | CGACATGGGC TCTTGGTGTG |
| | GCTGTACCCG AGAACCACAC |
| | prM Hypr |
| | ---------------------------------------------------------------------------------------- |
| | • D S L S Y E C V T I D Q G E E P V D V D C F C R N V |
| 401 | ATGATAGCCT TTCTTATGAG TGCGTAACCA TAGATCAAGG TGAGGAACCT GTTGACGTTG ATTGCTTCTG CCGAAACGTG |
| | TACTATCGGA AAGAATACTC ACGCATTGGT ATCTAGTTCC ACTCCTTGGA CAACTGCAAC TAACGAAGAC GGCTTTGCAC |
| | prM Hypr |
| | --------------------- |
| | D G V Y L E Y • |
| | GATGGGGTGT ATCTCGAATA |
| | CTACCCCACA TAGAGCTTAT |
| | prM Hypr |
| | ---------------------------------------------------------------------------------------- |
| | • G R C G K Q E G S R T R R S V L I P S H A Q G E L T G |
| 501 | TGGACGGTGT GGTAAACAAG AAGGAAGCAG AACCAGACGC TCAGTGCTTA TACCCTCCCA CGCTCAAGGA GAGCTGACCG |
| | ACCTGCCACA CCATTTGTTC TTCCTTCGTC TTGGTCTGCG AGTCACGAAT ATGGGAGGGT GCGAGTTCCT CTCGACTGGC |
| | prM Hypr |
| | --------------------- |
| | R G H K W L |
| | GACGGGGACA TAAATGGTTG |
| | CTGCCCCTGT ATTTACCAAC |
| | prM Hypr |
| | ---------------------------------------------------------------------------------------- |
| | E G D S L R T H L T R V E G W V W K N R L L A L A M V |
| 601 | GAGGGCGACT CACTCCGAAC ACATTTGACC CGCGTCGAGG GCTGGGTCTG GAAAAATCGG CTGTTGGCCC TCGCTATGGT |
| | CTCCCGCTGA GTGAGGCTTG TGTAAACTGG GCGCAGCTCC CGACCCAGAC CTTTTTAGCC GACAACCGGG AGCGATACCA |
| | prM Hypr |
| | --------------------- |
| | T V V W L T L • |
| | GACAGTCGTT TGGCTCACGC |
| | CTGTCAGCAA ACCGAGTGCG |
| | E Hypr |
| | ----------------- |
| | prM Hypr |
| | ----------------------------------------------------------------------- |
| | • E S V V T R V A V L V V L L C L A P V Y A S R C T H |
| 701 | TGGAGTCTGT GGTTACTCGC GTGGCAGTGC TGGTGGTGCT CCTCTGTCTT GCCCCTGTCT ACGCGTCCAG GTGTACTCAT |
| | ACCTCAGACA CCAATGAGCG CACCGTCACG ACCACCACGA GGAGACAGAA CGGGGACAGA TGCGCAGGTC CACATGAGTA |
| | E Hypr |
| | --------------------- |
| | L E N R D F V • |
| | TTGGAAAACA GAGATTTTGT |
| | AACCTTTTGT CTCTAAAACA |
| | E Hypr |
| | ---------------------------------------------------------------------------------------- |
| | • T G T Q G T T R V T L V L E L G G C V T I T A E G K P |
| 801 | CACCGGCACC CAGGGGACGA CTCGGGTAAC CCTGGTGCTT GAACTGGGTG GTTGCGTTAC TATTACCGCT GAGGGCAAAC |
| | GTGGCCGTGG GTCCCCTGCT GAGCCCATTG GGACCACGAA CTTGACCCAC CAACGCAATG ATAATGGCGA CTCCCGTTTG |
| | E Hypr |
| | --------------------- |
| | S M D V W L |
| | CCTCTATGGA TGTGTGGCTG |
| | GGAGATACCT ACACACCGAC |
| | E Hypr |
| | ---------------------------------------------------------------------------------------- |
| | D A I Y Q E N P A Q T R E Y C L H A K L S D T K V A K |
| 901 | GATGCAATCT ATCAGGAGAA TCCCGCACAA ACCAGGGAAT ATTGCCTTCA CGCAAAGCTG TCCGATACAA AGGTCGCGGC |
| | CTACGTTAGA TAGTCCTCTT AGGGCGTGTT TGGTCCCTTA TAACGGAAGT GCGTTTCGAC AGGCTATGTT TCCAGCGCCG |
| | E Hypr |
| | --------------------- |
| | R C P T M G P • |
| | TAGGTGCCCA ACAATGGGAC |
| | ATCCACGGGT TGTTACCCTG |
| | E Hypr |
| | ---------------------------------------------------------------------------------------- |
| | • A T L A E E H Q G G T V C K R D Q S D R G W G N H C |
| 1001 | CGGCCACCCT GGCGGAGGAA CATCAGGGAG GTACAGTGTG CAAACGGGAC CAGAGTGATA GAGGCTGGGG TAATCACTGC |
| | GCCGGTGGGA CCGCCTCCTT GTAGTCCCTC CATGTCACAC GTTTGCCCTG GTCTCACTAT CTCCGACCCC ATTAGTGACG |
| | E Hypr |
| | --------------------- |
| | G L F G K G S • |
| | GGCCTGTTCG GCAAAGGAAG |
| | CCGGACAAGC CGTTTCCTTC |
| | E Hypr |
| | ---------------------------------------------------------------------------------------- |
| | • I V A C V K A A C E A K K K A T G H V Y D A N K I V Y |
| 1101 | TATTGTCGCT TGCGTCAAGG CAGCCTGTGA GGCCAAAAAG AAGGCTACTG GGCACGTCTA TGACGCCAAC AAGATCGTTT |
| | ATAACAGCGA ACGCAGTTCC GTCGGACACT CCGGTTTTTC TTCCGATGAC CCGTGCAGAT ACTGCGGTTG TTCTAGCAAA |
| | E Hypr |
| | --------------------- |
| | T V K V E P |
| | ATACAGTGAA AGTGGAACCA |
| | TATGTCACTT TCACCTTGGT |
| | E Hypr |
| | ---------------------------------------------------------------------------------------- |
| | H T G D Y V A A N E T H S G R K T A S F T V S S E K T |
| 1201 | CACACAGGGG ATTACGTGGC GGCCAACGAG ACTCATTCCG GTCGCAAAAC GGCCAGCTTC ACCGTGTCAT CCGAAAAGAC |
| | GTGTGTCCCC TAATGCACCG CCGGTTGCTC TGAGTAAGGC CAGCGTTTTG CCGGTCGAAG TGGCACAGTA GGCTTTTCTG |
| | E Hypr |
| | --------------------- |
| | I L T M G E Y • |
| | CATCCTCACT ATGGGGGAGT |
| | GTAGGAGTGA TACCCCCTCA |
| | E Hypr |
| | ---------------------------------------------------------------------------------------- |
| | • G D V S L L C R V A S G V D L A Q T V I L E L D K T |
| 1301 | ATGGCGACGT TTCTCTGCTC TGCCGGGTGG CTAGCGGAGT CGACCTGGCC CAGACAGTCA TCCTGGAACT GGATAAAACA |
| | TACCGCTGCA AAGAGACGAG ACGGCCCACC GATCGCCTCA GCTGGACCGG GTCTGTCAGT AGGACCTTGA CCTATTTTGT |
| | E Hypr |
| | --------------------- |
| | V E H L P T A • |
| | GTTGAGCATC TGCCTACCGC |
| | CAACTCGTAG ACGGATGGCG |
| | E Hypr |
| | ---------------------------------------------------------------------------------------- |
| | • W Q V H R D W F N D L A L P W K H E G A R N W N N A E |
| 1401 | TTGGCAGGTG CACAGGGATT GGTTTAACGA CCTTGCCCTG CCATGGAAAC ATGAAGGAGC GAGAAACTGG AATAATGCAG |
| | AACCGTCCAC GTGTCCCTAA CCAAATTGCT GGAACGGGAC GGTACCTTTG TACTTCCTCG CTCTTTGACC TTATTACGTC |
| | E Hypr |
| | --------------------- |
| | R L V E F G |
| | AGCGACTCGT AGAATTCGGT |
| | TCGCTGAGCA TCTTAAGCCA |
| | E Hypr |
| | ---------------------------------------------------------------------------------------- |
| | A P H A V K M D V Y N L G D Q T G V L L K A L A G V P |
| 1501 | GCCCCTCATG CCGTGAAGAT GGACGTCTAC AATCTGGGTG ATCAGACCGG CGTTCTCCTT AAAGCTCTCG CTGGCGTACC |
| | CGGGGAGTAC GGCACTTCTA CCTGCAGATG TTAGACCCAC TAGTCTGGCC GCAAGAGGAA TTTCGAGAGC GACCGCATGG |
| | E Hypr |
| | --------------------- |
| | V A H I E G T • |
| | AGTTGCCCAC ATCGAAGGAA |
| | TCAACGGGTG TAGCTTCCTT |
| | E Hypr |
| | ---------------------------------------------------------------------------------------- |
| | • K Y H L K S G H V T C E V G L E K L K M K G L T Y T |
| 1601 | CGAAGTACCA CCTGAAGTCA GGCCATGTAA CTTGCGAGGT GGGCCTGGAG AAGTTGAAAA TGAAAGGTCT TACGTACACA |
| | GCTTCATGGT GGACTTCAGT CCGGTACATT GAACGCTCCA CCCGGACCTC TTCAACTTTT ACTTTCCAGA ATGCATGTGT |
| | E Hypr |
| | --------------------- |
| | M C D K T K F • |
| | ATGTGTGACA AGACCAAGTT |
| | TACACACTGT TCTGGTTCAA |
| | E Hypr |
| | ---------------------------------------------------------------------------------------- |
| | • T W K R A P T D S G H D T V V M E V T F S G T K P C R |
| 1701 | CACATGGAAG AGGGCCCCCA CAGATAGCGG CCACGATACT GTGGTGATGG AGGTGACCTT TTCTGGAACA AAACCCTGCA |
| | GTGTACCTTC TCCCGGGGGT GTCTATCGCC GGTGCTATGA CACCACTACC TCCACTGGAA AAGACCTTGT TTTGGGACGT |
| | E Hypr |
| | --------------------- |
| | I P V R A V |
| | GAATACCCGT GCGGGCTGTA |
| | CTTATGGGCA CGCCCGACAT |
| | E Hypr |
| | ---------------------------------------------------------------------------------------- |
| | A H G S P D V N V A M L I T P N P T I E N N G G G F I |
| 1801 | GCTCACGGAT CTCCCGATGT CAATGTTGCT ATGCTGATTA CACCTAACCC TACCATCGAG AATAACGGTG GTGGTTTTAT |
| | CGAGTGCCTA GAGGGCTACA GTTACAACGA TACGACTAAT GTGGATTGGG ATGGTAGCTC TTATTGCCAC CACCAAAATA |
| | E Hypr |
| | --------------------- |
| | E M Q L P P G • |
| | TGAGATGCAG CTTCCGCCAG |
| | ACTCTACGTC GAAGGCGGTC |
| | E Hypr |
| | ---------------------------------------------------------------------------------------- |
| | • D N I I Y V G E L S Y Q W F Q K G S S I G R V F Q K |
| 1901 | GCGATAACAT CATCTACGTG GGCGAACTCT CTTACCAGTG GTTTCAGAAA GGGAGTTCAA TTGGGCGGGT CTTCCAAAAA |
| | CGCTATTGTA GTAGATGCAC CCGCTTGAGA GAATGGTCAC CAAAGTCTTT CCCTCAAGTT AACCCGCCCA GAAGGTTTTT |
| | E Hypr |
| | --------------------- |
| | T K K G I E R • |
| | ACGAAGAAGG GAATCGAACG |
| | TGCTTCTTCC CTTAGCTTGC |
| | E Hypr |
| | ---------------------------------------------------------------------------------------- |
| | • L T V I G E H A W D F G S A G G F L S S I G K A L H T |
| 2001 | ATTGACGGTT ATCGGCGAGC ACGCATGGGA TTTTGGTTCC GCAGGGGGAT TCCTGTCTTC TATTGGTAAG GCACTGCATA |
| | TAACTGCCAA TAGCCGCTCG TGCGTACCCT AAAACCAAGG CGTCCCCCTA AGGACAGAAG ATAACCATTC CGTGACGTAT |
| | E Hypr |
| | --------------------- |
| | V L G G A F |
| | CCGTGCTGGG GGGCGCATTC |
| | GGCACGACCC CCCGCGTAAG |
| | E Hypr |
| | ---------------------------------------------------------------------------------------- |
| | N S I F G G V G F L P K L L L G V A L A W L G L N M R |
| 2101 | AATTCTATTT TCGGGGGCGT GGGGTTCCTG CCTAAACTCC TGCTGGGAGT AGCCCTGGCC TGGTTGGGAC TGAATATGCG |
| | TTAAGATAAA AGCCCCCGCA CCCCAAGGAC GGATTTGAGG ACGACCCTCA TCGGGACCGG ACCAACCCTG ACTTATACGC |
| | E Hypr |
| | --------------------- |
| | N P T M S M S • |
| | GAATCCGACG ATGTCCATGT |
| | CTTAGGCTGC TACAGGTACA |
| | E Hypr |
| | ---------------------------------------------------------- |
| | WNV NS1 protein |
| | ------------------------------ |
| | • F L L A G V L V L A M T L G V G A D T G C A I D I S |
| 2201 | CATTCCTCTT GGCCGGCGTG CTTGTACTGG CCATGACACT GGGCGTTGGC GCCGACACTG GGTGTGCCAT AGACATCAGC |
| | GTAAGGAGAA CCGGCCGCAC GAACATGACC GGTACTGTGA CCCGCAACCG CGGCTGTGAC CCACACGGTA TCTGTAGTCG |
| | WNV NS1 |
| | protein |
| | ------ |
| | R Q |
| | CGGCAA |
| | GCCGTT |

PIV-WN/TBEV Hypr with WNV signal (p40)

| | |
|---|---|
| | 5′ UTR |
| | ---------------------------------------------------------------------------------------- |
| 1 | AGTAGTTCGC CTGTGTGAGC TGACAAACTT AGTAGTGTTT GTGAGGATTA ACAACAATTA ACACAGTGCG AGCTGTTTCT |
| | TCATCAAGCG GACACACTCG ACTGTTTGAA TCATCACAAA CACTCCTAAT TGTTGTTAAT TGTGTCACGC TCGACAAAGA |
| | deleted C |
| | ---- |
| | 5′ UTR |
| | ----------------- |
| | M S • |
| | TAGCACGAAG ATCTCGATGT |
| | ATCGTGCTTC TAGAGCTACA |
| | WNV deleted C |
| | ---------------------------------------------------------------------------------------- |
| | • K K P G G P G K S R A V Y L L K R G M P R V L S L I |
| 101 | CTAAGAAACC AGGAGGGCCC GGCAAGAGCC GGGCTGTCTA TTTGCTAAAA CGCGGAATGC CCCGCGTGTT GTCCTTGATT |
| | GATTCTTTGG TCCTCCCGGG CCGTTCTCGG CCCGACAGAT AAACGATTTT GCGCCTTACG GGGCGCACAA CAGGAACTAA |
| | WNV deleted C |
| | --------------------- |
| | G L K R S S K • |
| | GGACTTAAGC GGAGCTCCAA |
| | CCTGAATTCG CCTCGAGGTT |
| | WNV signal |
| | ----------------------------------------------------------- |
| | WNV deleted C prM Hypr |
| | -------------- --------------- |
| | • Q K K R G G K T G I A V M I G M L A C V G A A T V R X |
| 201 | GCAAAAGAAA CGCGGGGGAA AGACAGGCAT AGCTGTGATG ATAGGCATGC TGGCTTGTGT CGGAGCAGCT ACCGTGCGAA |
| | CGTTTTCTTT GCGCCCCCTT TCTGTCCGTA TCGACACTAC TATCCGTACG ACCGAACACA GCCTCGTCGA TGGCACGCTT |
| | prM Hypr |
| | -------------------- |
| | E R D G S T |
| | AAGAACGCGA CGGAAGCACC |
| | TTCTTGCGCT GCCTTCGTGG |
| | prM Hypr |
| | ---------------------------------------------------------------------------------------- |
| | V I R A E G K D A A T Q V R V E N G T C V I L A T D M |
| 301 | GTGATAAGGG CTGAGGGTAA GGATGCGGCT ACGCAGGTGA GAGTAGAGAA TGGCACTTGC GTAATACTCG CGACTGATAT |
| | CACTATTCCC GACTCCCATT CCTACGCCGA TGCGTCCACT CTCATCTCTT ACCGTGAACG CATTATGAGC GCTGACTATA |
| | prM Hypr |
| | --------------------- |
| | G S W C D D S • |
| | GGGATCCTGG TGTGACGATA |
| | CCCTAGGACC ACACTGCTAT |
| | prM Hypr |
| | ---------------------------------------------------------------------------------------- |
| | • L S Y E C V T I D Q G E E P V D V D C F C R N V D G |
| 401 | GCCTCAGTTA TGAATGCGTA ACAATAGACC AGGGCGAAGA ACCTGTGGAC GTTGACTGTT TCTGTAGAAA TGTGGATGGC |
| | CGGAGTCAAT ACTTACGCAT TGTTATCTGG TCCCGCTTCT TGGACACCTG CAACTGACAA AGACATCTTT ACACCTACCG |
| | prM Hypr |
| | --------------------- |
| | V Y L E Y G R • |
| | GTTTATCTGG AGTACGGCCG |
| | CAAATAGACC TCATGCCGGC |
| | prM Hypr |
| | ---------------------------------------------------------------------------------------- |
| | • C G K Q E G S R T R R S V L I P S H A Q G E L T G R G |
| 501 | CTGTGGAAAA CAGGAGGGCT CACGAACTCG AAGATCTGTG CTGATTCCAA GTCACGCGCA AGGAGAGTTG ACCGGTAGAG |
| | GACACCTTTT GTCCTCCCGA GTGCTTGAGC TTCTAGACAC GACTAAGGTT CAGTGCGCGT TCCTCTCAAC TGGCCATCTC |
| | prM Hypr |
| | --------------------- |
| | H K W L E G |
| | GCCACAAGTG GCTTGAAGGG |
| | CGGTGTTCAC CGAACTTCCC |
| | prM Hypr |
| | ---------------------------------------------------------------------------------------- |
| | D S L R T H L T R V E G W V W K N R L L A L A M V T V |
| 601 | GACTCATTGA GGACCCACCT GACTAGGGTG GAGGGTTGGG TTTGGAAGAA TCGGTTGCTC GCGCTCGCTA TGGTCACCGT |
| | CTGAGTAACT CCTGGGTGGA CTGATCCCAC CTCCCAACCC AAACCTTCTT AGCCAACGAG CGCGAGCGAT ACCAGTGGCA |
| | prM Hypr |
| | --------------------- |
| | V W L T L E S • |
| | CGTGTGGCTG ACACTGGAGA |
| | GCACACCGAC TGTGACCTCT |
| | E Hypr |
| | ------------------------ |
| | prM Hypr |
| | ---------------------------------------------------------------- |
| | • V V T R V A V L V V L L C L A P V Y A S R C T H L E |
| 701 | GTGTCGTGAC TCGGGTTGCT GTGTTGGTTG TCCTCCTCTG TTTGGCCCCA GTGTACGCGT CCAGGTGTAC TCATTTGGAA |
| | CACAGCACTG AGCCCAACGA CACAACCAAC AGGAGGAGAC AAACCGGGGT CACATGCGCA GGTCCACATG AGTAAACCTT |
| | E Hypr |
| | --------------------- |
| | N R D F V T G • |
| | AACAGAGATT TTGTCACCGG |
| | TTGTCTCTAA AACAGTGGCC |
| | E Hypr |
| | ---------------------------------------------------------------------------------------- |
| | • T Q G T T R V T L V L E L G G C V T I T A E G K P S M |
| 801 | CACCCAGGGG ACGACTCGGG TAACCCTGGT GCTTGAACTG GGTGGTTGCG TTACTATTAC CGCTGAGGGC AAACCCTCTA |
| | GTGGGTCCCC TGCTGAGCCC ATTGGGACCA CGAACTTGAC CCACCAACGC AATGATAATG GCGACTCCCG TTTGGGAGAT |
| | E Hypr |
| | --------------------- |
| | D V W L D A |
| | TGGATGTGTG GCTGGATGCA |
| | ACCTACACAC CGACCTACGT |
| | E Hypr |
| | ---------------------------------------------------------------------------------------- |
| | I Y Q E N P A Q T R E Y C L H A K L S D T K V A A R C |
| 901 | ATCTATCAGG AGAATCCCGC ACAAACCAGG GAATATTGCC TTCACGCAAA GCTGTCCGAT ACAAAGGTCG CGGCTAGGTG |
| | TAGATAGTCC TCTTAGGGCG TGTTTGGTCC CTTATAACGG AAGTGCGTTT CGACAGGCTA TGTTTCCAGC GCCGATCCAC |
| | E Hypr |
| | --------------------- |
| | P T M G P A T • |
| | CCCAACAATG GGACCGGCCA |
| | GGGTTGTTAC CCTGGCCGGT |
| | E Hypr |
| | ---------------------------------------------------------------------------------------- |
| | • L A E E H Q G G T V C K R D Q S D R G W G N H C G L |
| 1001 | CCCTGGCGGA GGAACATCAG GGAGGTACAG TGTGCAAACG GGACCAGAGT GATAGAGGCT GGGGTAATCA CTGCGGCCTG |
| | GGGACCGCCT CCTTGTAGTC CCTCCATGTC ACACGTTTGC CCTGGTCTCA CTATCTCCGA CCCCATTAGT GACGCCGGAC |
| | E Hypr |
| | --------------------- |
| | F G K G S I V • |
| | TTCGGCAAAG GAAGTATTGT |
| | AAGCCGTTTC CTTCATAACA |
| | E Hypr |
| | ---------------------------------------------------------------------------------------- |
| | • A C V K A A C E A K K K A T G H V Y D A N K I V Y T V |
| 1101 | CGCTTGCGTC AAGGCAGCCT GTGAGGCCAA AAAGAAGGCT ACTGGGCACG TCTATGACGC CAACAAGATC GTTTATACAG |
| | GCGAACGCAG TTCCGTCGGA CACTCCGGTT TTTCTTCCGA TGACCCGTGC AGATACTGCG GTTGTTCTAG CAAATATGTC |
| | E Hypr |
| | --------------------- |
| | K V E P H T |
| | TGAAAGTGGA ACCACACACA |
| | ACTTTCACCT TGGTGTGTGT |
| | E Hypr |
| | ---------------------------------------------------------------------------------------- |
| | G D Y V A A N E T H S G R K T A S F T V S S E K T I L |
| 1201 | GGGGATTACG TGGCGGCCAA CGAGACTCAT TCCGGTCGCA AAACGGCCAG CTTCACCGTG TCATCCGAAA AGACCATCCT |
| | CCCCTAATGC ACCGCCGGTT GCTCTGAGTA AGGCCAGCGT TTTGCCGGTC GAAGTGGCAC AGTAGGCTTT TCTGGTAGGA |
| | E Hypr |
| | --------------------- |
| | T M G E Y G D • |
| | CACTATGGGG GAGTATGGCG |
| | GTGATACCCC CTCATACCGC |
| | E Hypr |
| | ---------------------------------------------------------------------------------------- |
| | • V S L L C R V A S G V D L A Q T V I L E L D K T V E |
| 1301 | ACGTTTCTCT GCTCTGCCGG GTGGCTAGCG GAGTCGACCT GGCCCAGACA GTCATCCTGG AACTGGATAA AACAGTTGAG |
| | TGCAAAGAGA CGAGACGGCC CACCGATCGC CTCAGCTGGA CCGGGTCTGT CAGTAGGACC TTGACCTATT TTGTCAACTC |
| | E Hypr |
| | --------------------- |
| | H L P T A W Q • |
| | CATCTGCCTA CCGCTTGGCA |
| | GTAGACGGAT GGCGAACCGT |
| | E Hypr |
| | ---------------------------------------------------------------------------------------- |
| | • V H R D W F N D L A L P W K H E G A R N W N N A E R L |
| 1401 | GGTGCACAGG GATTGGTTTA ACGACCTTGC CCTGCCATGG AAACATGAAG GAGCGAGAAA CTGGAATAAT GCAGAGCGAC |
| | CCACGTGTCC CTAACCAAAT TGCTGGAACG GGACGGTACC TTTGTACTTC CTCGCTCTTT GACCTTATTA CGTCTCGCTG |
| | E Hypr |
| | --------------------- |
| | V E F G A P |
| | TCGTAGAATT CGGTGCCCCT |
| | AGCATCTTAA GCCACGGGGA |
| | E Hypr |
| | ---------------------------------------------------------------------------------------- |
| | H A V K M D V Y N L G D Q T G V L L K A L A G V P V A |
| 1501 | CATGCCGTGA AGATGGACGT CTACAATCTG GGTGATCAGA CCGGCGTTCT CCTTAAAGCT CTCGCTGGCG TACCAGTTGC |
| | GTACGGCACT TCTACCTGCA GATGTTAGAC CCACTAGTCT GGCCGCAAGA GGAATTTCGA GAGCGACCGC ATGGTCAACG |
| | E Hypr |
| | --------------------- |
| | H I E G T K Y • |
| | CCACATCGAA GGAACGAAGT |
| | GGTGTAGCTT CCTTGCTTCA |
| | E Hypr |
| | ---------------------------------------------------------------------------------------- |
| | • H L K S G H V T C E V G L E K L K M K G L T Y T M C |
| 1601 | ACCACCTGAA GTCAGGCCAT GTAACTTGCG AGGTGGGCCT GGAGAAGTTG AAAATGAAAG GTCTTACGTA CACAATGTGT |
| | TGGTGGACTT CAGTCCGGTA CATTGAACGC TCCACCCGGA CCTCTTCAAC TTTTACTTTC CAGAATGCAT GTGTTACACA |
| | E Hypr |
| | --------------------- |
| | D K T K F T W • |
| | GACAAGACCA AGTTCACATG |
| | CTGTTCTGGT TCAAGTGTAC |
| | E Hypr |
| | ---------------------------------------------------------------------------------------- |
| | • K R A P T D S G H D T V V M E V T F S G T K P C R I P |
| 1701 | GAAGAGGGCC CCCACAGATA GCGGCCACGA TACTGTGGTG ATGGAGGTGA CCTTTTCTGG AACAAAACCC TGCAGAATAC |
| | CTTCTCCCGG GGGTGTCTAT CGCCGGTGCT ATGACACCAC TACCTCCACT GGAAAAGACC TTGTTTTGGG ACGTCTTATG |
| | E Hypr |
| | --------------------- |
| | V R A V A H |
| | CCGTGCGGGC TGTAGCTCAC |
| | GGCACGCCCG ACATCGAGTG |
| | E Hypr |
| | ---------------------------------------------------------------------------------------- |
| | G S P D V N V A M L I T P N P T I E N N G G G F I E M |
| 1801 | GGATCTCCCG ATGTCAATGT TGCTATGCTG ATTACACCTA ACCCTACCAT CGAGAATAAC GGTGGTGGTT TTATTGAGAT |
| | CCTAGAGGGC TACAGTTACA ACGATACGAC TAATGTGGAT TGGGATGGTA GCTCTTATTG CCACCACCAA AATAACTCTA |
| | E Hypr |
| | --------------------- |
| | Q L P P G D N • |
| | GCAGCTTCCG CCAGGCGATA |
| | CGTCGAAGGC GGTCCGCTAT |
| | E Hypr |
| | ---------------------------------------------------------------------------------------- |
| | • I I Y V G E L S Y Q W F Q K G S S I G R V F Q K T K |
| 1901 | ACATCATCTA CGTGGGCGAA CTCTCTTACC AGTGGTTTCA GAAAGGGAGT TCAATTGGGC GGGTCTTCCA AAAAACGAAG |
| | TGTAGTAGAT GCACCCGCTT GAGAGAATGG TCACCAAAGT CTTTCCCTCA AGTTAACCCG CCCAGAAGGT TTTTTGCTTC |
| | E Hypr |
| | --------------------- |
| | K G I E R L T • |
| | AAGGGAATCG AACGATTGAC |
| | TTCCCTTAGC TTGCTAACTG |
| | E Hypr |
| | ---------------------------------------------------------------------------------------- |
| | • V I G E H A W D F G S A G G F L S S I G K A L H T V L |
| 2001 | CGTTATCGGC GAGCACGCAT GGGATTTTGG TTCCGCAGGG GGATTCCTGT CTTCTATTGG TAAGGCACTG CATACCGTGC |
| | CCAATAGCCG CTCGTGCGTA CCCTAAAACC AAGGCGTCCC CCTAAGGACA GAAGATAACC ATTCCGTGAC GTATGGCACG |
| | E Hypr |
| | --------------------- |
| | G G A F N S |
| | TGGGGGGCGC ATTCAATTCT |
| | ACCCCCCGCG TAAGTTAAGA |
| | E Hypr |
| | ---------------------------------------------------------------------------------------- |
| | I F G G V G F L P K L L L G V A L A W L G L N M R N P |
| 2101 | ATTTTCGGGG GCGTGGGGTT CCTGCCTAAA CTCCTGCTGG GAGTAGCCCT GGCCTGGTTG GGACTGAATA TGCGGAATCC |
| | TAAAAGCCCC CGCACCCCAA GGACGGATTT GAGGACGACC CTCATCGGGA CCGGACCAAC CCTGACTTAT ACGCCTTAGG |
| | E Hypr |
| | --------------------- |
| | T M S M S F L • |
| | GACGATGTCC ATGTCATTCC |
| | CTGCTACAGG TACAGTAAGG |
| | E Hypr |
| | --------------------------------------------------- |
| | WNV NS1 protein |
| | ------------------------------------- |
| | • L A G V L V L A M T L G V G A D T G C A I D I S R Q |
| 2201 | TCTTGGCCGG CGTGCTTGTA CTGGCCATGA CACTGGGCGT TGGCGCCGAC ACTGGGTGTG CCATAGACAT CAGCCGGCAA |
| | AGAACCGGCC GCACGAACAT GACCGGTACT GTGACCCGCA ACCGCGGCTG TGACCCACAC GGTATCTGTA GTCGGCCGTT |

SEQUENCE APPENDIX 4
WN PIV constructs expressing rabies virus G protein.
WN (ΔCprME)-Rabies PIV sequence (partial)

| | |
|---|---|
| | 5′ UTR |
| | ---------------------------------------------------------------------------------------- |
| 1 | AGTAGTTCGC CTGTGTGAGC TGACAAACTT AGTAGTGTTT GTGAGGATTA ACAACAATTA ACACAGTGCG AGCTGTTTCT |
| | TCATCAAGCG GACACACTCG ACTGTTTGAA TCATCACAAA CACTCCTAAT TGTTGTTAAT TGTGTCACGC TCGACAAAGA |
| | N-terminus of C |
| | ---- |
| | 5′ UTR |
| | ----------------- |
| | M S • |
| | ---- |
| | TAGCACGAAG ATCTCGATGT |
| | ATCGTGCTTC TAGAGCTACA |
| | N-terminus of C |
| | ---------------------------------------------------------------------------------------- |
| | • K K P G G P G K S R A V Y L L K R G M P R V L S L I |
| | ---------------------------------------------------------------------------------------- |
| 101 | CTAAGAAACC AGGAGGGCCC GGCAAGAGCC GGGCTGTCTA TTTGCTAAAA CGCGGAATGC CCCGCGTGTT GTCCTTGATT |
| | GATTCTTTGG TCCTCCCGGG CCGTTCTCGG CCCGACAGAT AAACGATTTT GCGCCTTACG GGGCGCACAA CAGGAACTAA |
| | N-terminus of C |
| | --------------------- |
| | G L K Q K K R • |
| | --------------------- |
| | GGACTTAAGC AAAAGAAGCG |
| | CCTGAATTCG TTTTCTTCGC |
| | N-terminus of C Rabies-G signal |
| | -- --------------------------------------------------------- |
| | partial C signal |
| | ---------------------------- |
| | • G G K T G I A V I V P Q A L L F V P L L V F P L C F G |
| | ---------------------------------------------------------------------------------------- |
| 201 | AGGGGGCAAG ACTGGTATAG CTGTGATCGT TCCTCAGGCT CTTTTGTTTG TACCCTTGCT GGTATTTCCC CTTTGCTTTG |
| | TCCCCCGTTC TGACCATATC GACACTAGCA AGGAGTCCGA GAAAACAAAC ATGGGAACGA CCATAAAGGG GAAACGAAAC |
| | Rabies-G signal |
| | -- |
| | Rabies-G protein |
| | ------------------- |
| | K F P I Y T |
| | --------------------- |
| | GTAAATTTCC TATCTATACC |
| | CATTTAAAGG ATAGATATGG |
| | Rabies-G protein |
| | ---------------------------------------------------------------------------------------- |
| | I P D K L G P W S P I D I H H L S C P N N L V V E D E |
| | ---------------------------------------------------------------------------------------- |
| 301 | ATCCCTGATA AGCTCGGGCC TTGGAGTCCC ATTGATATTC ACCATTTGAG CTGCCCAAAC AACCTCGTCG TTGAGGATGA |
| | TAGGGACTAT TCGAGCCCGG AACCTCAGGG TAACTATAAG TGGTAAACTC GACGGGTTTG TTGGAGCAGC AACTCCTACT |
| | Rabies-G protein |
| | --------------------- |
| | G C T N L S G • |
| | --------------------- |
| | AGGGTGCACT AATCTTTCTG |
| | TCCCACGTGA TTAGAAAGAC |
| | Rabies-G protein |
| | ---------------------------------------------------------------------------------------- |
| | • F S Y M E L K V G Y I S A I K M N G F T C T G V V T |
| | ---------------------------------------------------------------------------------------- |
| 401 | GATTTTCCTA CATGGAGTTG AAAGTGGGCT ATATTTCAGC CATTAAGATG AACGGCTTTA CTTGTACAGG AGTCGTGACC |
| | CTAAAAGGAT GTACCTCAAC TTTCACCCGA TATAAAGTCG GTAATTCTAC TTGCCGAAAT GAACATGTCC TCAGCACTGG |
| | Rabies-G protein |
| | --------------------- |
| | E A E T Y T N • |
| | --------------------- |
| | GAAGCCGAGA CATATACAAA |
| | CTTCGGCTCT GTATATGTTT |
| | Rabies-G protein |
| | ---------------------------------------------------------------------------------------- |
| | • F V G Y V T T T F K R K H F R P T P D A C R A A Y N W |
| | ---------------------------------------------------------------------------------------- |
| 501 | TTTCGTGGGA TACGTCACCA CCACCTTCAA GAGAAAACAC TTCCGCCCAA CGCCTGACGC TTGTCGGGCC GCTTACAACT |
| | AAAGCACCCT ATGCAGTGGT GGTGGAAGTT CTCTTTTGTG AAGGCGGGTT GCGGACTGCG AACAGCCCGG CGAATGTTGA |
| | Rabies-G protein |
| | --------------------- |
| | K M A G D P |
| | --------------------- |
| | GGAAGATGGC AGGAGATCCT |
| | CCTTCTACCG TCCTCTAGGA |
| | Rabies-G protein |
| | ---------------------------------------------------------------------------------------- |
| | R Y E E S L H N P Y P D Y H W L R T V K T T K E S L V |
| | ---------------------------------------------------------------------------------------- |
| 601 | CGATATGAAG AATCTCTGCA CAACCCGTAT CCTGATTACC ATTGGCTGCG GACAGTCAAG ACTACCAAGG AGAGTCTGGT |
| | GCTATACTTC TTAGAGACGT GTTGGGCATA GGACTAATGG TAACCGACGC CTGTCAGTTC TGATGGTTCC TCTCAGACCA |
| | Rabies-G protein |
| | --------------------- |
| | X I S P S V A • |
| | --------------------- |
| | CATTATATCA CCAAGCGTGG |
| | GTAATATAGT GGTTCGCACC |
| | Rabies-G protein |
| | ---------------------------------------------------------------------------------------- |
| | • D L D P Y D R S L H S R V F P G G N C S G V A V S S |
| | ---------------------------------------------------------------------------------------- |
| 701 | CCGATCTTGA TCCTTATGAT AGATCCCTGC ACAGTAGGGT TTTTCCTGGC GGGAATTGTA GCGGTGTTGC AGTATCAAGT |
| | GGCTAGAACT AGGAATACTA TCTAGGGACG TGTCATCCCA AAAAGGACCG CCCTTAACAT CGCCACAACG TCATAGTTCA |
| | Rabies-G protein |
| | --------------------- |
| | T Y C S T N H • |
| | --------------------- |
| | ACCTACTGCT CCACTAACCA |
| | TGGATGACGA GGTGATTGGT |
| | Rabies-G protein |
| | ---------------------------------------------------------------------------------------- |
| | • D Y T I W M P E N P R L G M S C D I F T N S R G K R A |
| | ---------------------------------------------------------------------------------------- |
| 801 | CGACTACACT ATATGGATGC CTGAGAACCC TCGACTCGGT ATGAGTTGCG ACATTTTTAC GAACTCACGG GGCAAGCGGG |
| | GCTGATGTGA TATACCTACG GACTCTTGGG AGCTGAGCCA TACTCAACGC TGTAAAAATG CTTGAGTGCC CCGTTCGCCC |
| | Rabies-G protein |
| | --------------------- |
| | S K G S E T |
| | --------------------- |
| | CATCTAAGGG GTCTGAAACA |
| | GTAGATTCCC CAGACTTTGT |
| | Rabies-G protein |
| | ---------------------------------------------------------------------------------------- |
| | C G F V D E R G L Y K S L K G A C K L K L C G V L G L |
| | ---------------------------------------------------------------------------------------- |
| 901 | TGCGGGTTTG TTGATGAGCG GGGGTTGTAT AAATCTCTTA AAGGCGCCTG TAAGCTGAAA CTCTGTGGCG TACTGGGGCT |
| | ACGCCCAAAC AACTACTCGC CCCCAACATA TTTAGAGAAT TTCCGCGGAC ATTCGACTTT GAGACACCGC ATGACCCCGA |
| | Rabies-G protein |
| | --------------------- |
| | R L M D G T W • |
| | --------------------- |
| | GCGCCTGATG GACGGCACAT |
| | CGCGGACTAC CTGCCGTGTA |
| | Rabies-G protein |
| | ---------------------------------------------------------------------------------------- |
| | • V A M Q T S N E T K W C P P G Q L V N L H D F R S D |
| | ---------------------------------------------------------------------------------------- |
| 1001 | GGGTGGCTAT GCAGACAAGC AATGAAACAA AGTGGTGTCC CCCTGGTCAG CTGGTTAATC TGCACGACTT TAGGTCTGAC |
| | CCCACCGATA CGTCTGTTCG TTACTTTGTT TCACCACAGG GGGACCAGTC GACCAATTAG ACGTGCTGAA ATCCAGACTG |
| | Rabies-G protein |
| | --------------------- |
| | E I E H L V V • |
| | --------------------- |
| | GAAATCGAGC ACCTTGTGGT |
| | CTTTAGCTCG TGGAACACCA |
| | Rabies-G protein |
| | ---------------------------------------------------------------------------------------- |
| | • E E L V K K R E E C L D A L E S I M T T K S V S F R R |
| | ---------------------------------------------------------------------------------------- |
| 1101 | GGAGGAACTG GTGAAGAAAC GCGAAGAGTG CCTGGACGCA CTTGAGAGTA TTATGACCAC CAAATCCGTT TCCTTCAGAA |
| | CCTCCTTGAC CACTTCTTTG CGCTTCTCAC GGACCTGCGT GAACTCTCAT AATACTGGTG GTTTAGGCAA AGGAAGTCTT |
| | Rabies-G protein |
| | --------------------- |
| | L S H L R K |
| | --------------------- |
| | GACTGAGCCA CCTGCGAAAG |
| | CTGACTCGGT GGACGCTTTC |
| | Rabies-G protein |
| | ---------------------------------------------------------------------------------------- |
| | L V P G F G K A Y T I F N K T L M E A D A H Y K S V R |
| | ---------------------------------------------------------------------------------------- |
| 1201 | CTGGTGCCAG GGTTCGGGAA GGCTTATACT ATTTTCAACA AGACTCTTAT GGAGGCGGAT GCCCATTATA AGTCAGTTAG |
| | GACCACGGTC CCAAGCCCTT CCGAATATGA TAAAAGTTGT TCTGAGAATA CCTCCGCCTA CGGGTAATAT TCAGTCAATC |
| | Rabies-G protein |
| | --------------------- |
| | T W N E I I P • |
| | --------------------- |
| | GACTTGGAAT GAGATAATTC |
| | CTGAACCTTA CTCTATTAAG |
| | Rabies-G protein |
| | ---------------------------------------------------------------------------------------- |
| | • S K G C L R V G G R C H P H V N G V F F N G I I L G |
| | ---------------------------------------------------------------------------------------- |
| 1301 | CCTCCAAAGG ATGTCTGAGA GTCGGTGGGA GATGCCACCC CCATGTCAAT GGGGTGTTCT TTAACGGAAT CATCCTGGGA |
| | GGAGGTTTCC TACAGACTCT CAGCCACCCT CTACGGTGGG GGTACAGTTA CCCCACAAGA AATTGCCTTA GTAGGACCCT |
| | Rabies-G protein |
| | --------------------- |
| | P D G N V L I • |
| | --------------------- |
| | CCTGACGGGA ACGTGCTGAT |
| | GGACTGCCCT TGCACGACTA |
| | Rabies-G protein |
| | ---------------------------------------------------------------------------------------- |
| | • P E M Q S S L L Q Q H M E L L V S S V I P L M H P L A |
| | ---------------------------------------------------------------------------------------- |
| 1401 | TCCCGAGATG CAATCTTCCC TTCTGCAGCA ACACATGGAA CTCCTGGTGT CTTCAGTGAT ACCCCTGATG CACCCACTGG |
| | AGGGCTCTAC GTTAGAAGGG AAGACGTCGT TGTGTACCTT GAGGACCACA GAAGTCACTA TGGGGACTAC GTGGGTGACC |
| | Rabies-G protein |
| | --------------------- |
| | D P S T V F |
| | --------------------- |
| | CCGACCCCAG CACTGTGTTC |
| | GGCTGGGGTC GTGACACAAG |
| | Rabies-G protein |
| | ---------------------------------------------------------------------------------------- |
| | K N G D E A E D F V E V H L P D V H E R I S G V D L G |
| | ---------------------------------------------------------------------------------------- |
| 1501 | AAAAATGGCG ATGAGGCCGA AGACTTTGTG GAAGTTCACC TGCCCGATGT ACACGAAAGG ATATCTGGAG TAGACCTGGG |
| | TTTTTACCGC TACTCCGGCT TCTGAAACAC CTTCAAGTGG ACGGGCTACA TGTGCTTTCC TATAGACCTC ATCTGGACCC |
| | Rabies-G protein |
| | --------------------- |
| | L P N W G K Y • |
| | --------------------- |
| | CCTTCCTAAT TGGGGTAAGT |
| | GGAAGGATTA ACCCCATTCA |
| | Rabies-G protein |
| | ---------------------------------------------------------------------------------------- |
| | • V L L S A G A L T A L M L I I F L M T C W R R V N R |
| | ---------------------------------------------------------------------------------------- |
| 1601 | ACGTGCTCCT GAGTGCGGGT GCCTTGACCG CTTTGATGCT GATCATTTTT CTGATGACCT GCTGGCGGAG GGTGAATCGC |
| | TGCACGAGGA CTCACGCCCA CGGAACTGGC GAAACTACGA CTAGTAAAAA GACTACTGGA CGACCGCCTC CCACTTAGCG |
| | Rabies-G protein |
| | --------------------- |
| | S E P T Q H N • |
| | --------------------- |
| | TCCGAGCCGA CACAGCACAA |
| | AGGCTCGGCT GTGTCGTGTT |
| | Rabies-G protein |
| | ---------------------------------------------------------------------------------------- |
| | • L R G T G R E V S V T P Q S G K I I S S W E S Y K S G |
| | ---------------------------------------------------------------------------------------- |
| 1701 | TCTCAGAGGG ACAGGCCGGG AAGTAAGTGT GACTCCGCAA TCTGGCAAGA TTATTAGTAG TTGGGAGAGT TACAAGTCTG |
| | AGAGTCTCCC TGTCCGGCCC TTCATTCACA CTGAGGCGTT AGACCGTTCT AATAATCATC AACCCTCTCA ATGTTCAGAC |
| | Rabies-G protein |
| | ------------------ |
| | FMDV 2A |
| | --- |
| | G E T G L N |
| | --------------------- |
| | GAGGAGAGAC TGGGTTGAAT |
| | CTCCTCTCTG ACCCAACTTA |
| | preNS1 signal |
| | ---------- |
| | FMDV 2A NS1 signal |
| | ---------------------------------------------------- -------------------------- |
| | F D L L K L A G D V E S N P G P A R D R S I A L T F L |
| | ---------------------------------------------------------------------------------------- |
| 1801 | TTTGATCTGC TCAAACTTGC AGGCGATGTA GAATCAAATC CTGGACCCGC CCGGGACAGG TCCATAGCTC TCACGTTTCT |
| | AAACTAGACG AGTTTGAACG TCCGCTACAT CTTAGTTTAG GACCTGGGCG GGCCCTGTCC AGGTATCGAG AGTGCAAAGA |
| | NS1 signal |
| | --------------------- |
| | A V G G V L L • |
| | --------------------- |
| | CGCAGTTGGA GGAGTTCTGC |
| | GCGTCAACCT CCTCAAGACG |
| | NS1 signal |
| | ---------------------------- |
| | NS1 |
| | ------------------------------------------------------------ |
| | • F L S V N V H A D T G C A I D I S R Q E L R C G S G |
| | ---------------------------------------------------------------------------------------- |
| 1901 | TCTTCCTCTC CGTGAACGTG CACGCTGACA CTGGGTGTGC CATAGACATC AGCCGGCAAG AGCTGAGATG TGGAAGTGGA |
| | AGAAGGAGAG GCACTTGCAC GTGCGACTGT GACCCACACG GTATCTGTAG TCGGCCGTTC TCGACTCTAC ACCTTCACCT |
| | NS1 |
| | --------------------- |
| | V F I H N D V • |
| | --------------------- |
| | GTGTTCATAC ACAATGATGT |
| | CACAAGTATG TGTTACTACA |

WN (ΔC)-Rabies G PIV sequence (partial).

| | |
|---|---|
| | 5′UTR |
| | ---------------------------------------------------------------------------------------- |
| 1 | AGTAGTTCGC CTGTGTGAGC TGACAAACTT AGTAGTGTTT GTGAGGATTA ACAACAATTA ACACAGTGCG AGCTGTTTCT |
| | TCATCAAGCG GACACACTCG ACTGTTTGAA TCATCACAAA CACTCCTAAT TGTTGTTAAT TGTGTCACGC TCGACAAAGA |
| | 5′UTR |
| | ----------------- |
| | N- |
| | terminus of C |
| | ---- |
| | M S • |
| | ---- |
| | TAGCACGAAG ATCTCGATGT |
| | ATCGTGCTTC TAGAGCTACA |
| | N-terminus of C |
| | ---------------------------------------------------------------------------------------- |
| | • K K P G G P G K S R A V N M L K R G M P R V L S L I |
| | ---------------------------------------------------------------------------------------- |
| 101 | CTAAGAAACC AGGAGGGCCC GGCAAGAGCC GGGCTGTCAA TATGCTAAAA CGCGGAATGC CCCGCGTGTT GTCCTTGATT |
| | GATTCTTTGG TCCTCCCGGG CCGTTCTCGG CCCGACAGTT ATACGATTTT GCGCCTTACG GGGCGCACAA CAGGAACTAA |
| | N-terminus of C |
| | --------------------- |
| | G L K Q K K R • |
| | --------------------- |
| | GGACTTAAGC AAAAGAAGCG |
| | CCTGAATTCG TTTTCTTCGC |
| | N-terminus of C |
| | -- |
| | partial C signal RAbies-G signal |
| | ---------------------------- ------------------------------------------------------------ |
| | • G G K T G I A V I V P Q A L L F V P L L V F P L C F G |
| | ------------------------------------------------------------------------------------------- |
| 201 | AGGGGGCAAG ACTGGTATAG CTGTGATCGT TCCTCAGGCT CTTTTGTTTG TACCCTTGCT GGTATTTCCC CTTTGCTTTG GT |
| | TCCCCCGTTC TGACCATATC GACACTAGCA AGGAGTCCGA GAAAACAAAC ATGGGAACGA CCATAAAGGG GAAAACAAAC CA |
| | Rabies-G protein |
| | ------------------- |
| | K F P I Y T |
| | ------------------- |
| | AAATTTCC TATCTATACC |
| | TTTAAAGG ATAGATATGG |
| | Rabies-G protein |
| | ---------------------------------------------------------------------------------------- |
| | I P D K L G P W S P I D I H H L S C P N N L V V E D E |
| | ---------------------------------------------------------------------------------------- |
| 301 | ATCCCTGATA AGCTCGGGCC TTGGAGTCCC ATTGATATTC ACCATTTGAG CTGCCCAAAC AACCTCGTCG TTGAGGATGA |
| | TAGGGACTAT TCGAGCCCGG AACCTCAGGG TAACTATAAG TGGTAAACTC GACGGGTTTG TTGGAGCAGC AACTCCTACT |
| | Rabies-G protein |
| | --------------------- |
| | G C T N L S G • |
| | --------------------- |
| | AGGGTGCACT AATCTTTCTG |
| | TCCCACGTGA TTAGAAAGAC |
| | Rabies-G protein |
| | ---------------------------------------------------------------------------------------- |
| | • F S Y M E L K V G Y I S A I K M N G F T C T G V V T |
| | ---------------------------------------------------------------------------------------- |
| 401 | GATTTTCCTA CATGGAGTTG AAAGTGGGCT ATATTTCAGC CATTAAGATG AACGGCTTTA CTTGTACAGG AGTCGTGACC |
| | CTAAAAGGAT GTACCTCAAC TTTCACCCGA TATAAAGTCG GTAATTCTAC TTGCCGAAAT GAACATGTCC TCAGCACTGG |
| | Rabies-G protein |
| | --------------------- |
| | E A E T Y T N • |
| | --------------------- |
| | GAAGCCGAGA CATATACAAA |
| | CTTCGGCTCT GTATATGTTT |
| | Rabies-G protein |
| | ---------------------------------------------------------------------------------------- |
| | • F V G Y V T T T F K R K H F R P T P D A C R A A Y N W |
| | ---------------------------------------------------------------------------------------- |
| 501 | TTTCGTGGGA TACGTCACCA CCACCTTCAA GAGAAAACAC TTCCGCCCAA CGCCTGACGC TTGTCGGGCC GCTTACAACT |
| | AAAGCACCCT ATGCAGTGGT GGTGGAAGTT CTCTTTTGTG AAGGCGGGTT GCGGACTGCG AACAGCCCGG CGAATGTTGA |
| | Rabies-G protein |
| | --------------------- |
| | K M A G D P |
| | --------------------- |
| | GGAAGATGGC AGGAGATCCT |
| | CCTTCTACCG TCCTCTAGGA |
| | Rabies-G protein |
| | ---------------------------------------------------------------------------------------- |
| | R Y E E S L H N P Y P D Y H W L R T V K T T K E S L V |
| | ---------------------------------------------------------------------------------------- |
| 601 | CGATATGAAG AATCTCTGCA CAACCCGTAT CCTGATTACC ATTGGCTGCG GACAGTCAAG ACTACCAAGG AGAGTCTGGT |
| | GCTATACTTC TTAGAGACGT GTTGGGCATA GGACTAATGG TAACCGACGC CTGTCAGTTC TGATGGTTCC TCTCAGACCA |
| | Rabies-G protein |
| | --------------------- |
| | I I S P S V A • |
| | --------------------- |
| | CATTATATCA CCAAGCGTGG |
| | GTAATATAGT GGTTCGCACC |
| | Rabies-G protein |
| | ---------------------------------------------------------------------------------------- |
| | • D L D P Y D R S L H S R V F P G G N C S G V A V S S |
| | ---------------------------------------------------------------------------------------- |
| 701 | CCGATCTTGA TCCTTATGAT AGATCCCTGC ACAGTAGGGT TTTTCCTGGC GGGAATTGTA GCGGTGTTGC AGTATCAAGT |
| | GGCTAGAACT AGGAATACTA TCTAGGGACG TGTCATCCCA AAAAGGACCG CCCTTAACAT CGCCACAACG TCATAGTTCA |
| | Rabies-G protein |
| | --------------------- |
| | T Y C S T N H • |
| | --------------------- |
| | ACCTACTGCT CCACTAACCA |
| | TGGATGACGA GGTGATTGGT |
| | Rabies-G protein |
| | ---------------------------------------------------------------------------------------- |
| | • D Y T I W M P E N P R L G M S C D I F T N S R G K R A |
| | ---------------------------------------------------------------------------------------- |
| 801 | CGACTACACT ATATGGATGC CTGAGAACCC TCGACTCGGT ATGAGTTGCG ACATTTTTAC GAACTCACGG GGCAAGCGGG |
| | GCTGATGTGA TATACCTACG GACTCTTGGG AGCTGAGCCA TACTCAACGC TGTAAAAATG CTTGAGTGCC CCGTTCGCCC |
| | Rabies-G protein |
| | --------------------- |
| | S K G S E T |
| | --------------------- |
| | CATCTAAGGG GTCTGAAACA |
| | GTAGATTCCC CAGACTTTGT |
| | Rabies-G protein |
| | ---------------------------------------------------------------------------------------- |
| | C G F V D E R G L Y K S L K G A C K L K L C G V L G L |
| | ---------------------------------------------------------------------------------------- |
| 901 | TGCGGGTTTG TTGATGAGCG GGGGTTGTAT AAATCTCTTA AAGGCGCCTG TAAGCTGAAA CTCTGTGGCG TACTGGGGCT |
| | ACGCCCAAAC AACTACTCGC CCCCAACATA TTTAGAGAAT TTCCGCGGAC ATTCGACTTT GAGACACCGC ATGACCCCGA |
| | Rabies-G protein |
| | --------------------- |
| | R L M D G T W • |
| | --------------------- |
| | GCGCCTGATG GACGGCACAT |
| | CGCGGACTAC CTGCCGTGTA |
| | Rabies-G protein |
| | ---------------------------------------------------------------------------------------- |
| | • V A M Q T S N E T K W C P P G Q L V N L H D F R S D |
| | ---------------------------------------------------------------------------------------- |
| 1001 | GGGTGGCTAT GCAGACAAGC AATGAAACAA AGTGGTGTCC CCCTGGTCAG CTGGTTAATC TGCACGACTT TAGGTCTGAC |
| | CCCACCGATA CGTCTGTTCG TTACTTTGTT TCACCACAGG GGGACCAGTC GACCAATTAG ACGTGCTGAA ATCCAGACTG |
| | Rabies-G protein |
| | --------------------- |
| | E I E H L V V • |
| | --------------------- |
| | GAAATCGAGC ACCTTGTGGT |
| | CTTTAGCTCG TGGAACACCA |
| | Rabies-G protein |
| | ---------------------------------------------------------------------------------------- |
| | • E E L V K K R E E C L D A L E S T M T T K S V S F R R |
| | ---------------------------------------------------------------------------------------- |
| 1101 | GGAGGAACTG GTGAAGAAAC GCGAAGAGTG CCTGGACGCA CTTGAGAGTA TTATGACCAC CAAATCCGTT TCCTTCAGAA |
| | CCTCCTTGAC CACTTCTTTG CGCTTCTCAC GGACCTGCGT GAACTCTCAT AATACTGGTG GTTTAGGCAA AGGAAGTCTT |
| | Rabies-G protein |
| | --------------------- |
| | L S H L R K |
| | --------------------- |
| | GACTGAGCCA CCTGCGAAAG |
| | CTGACTCGGT GGACGCTTTC |
| | Rabies-G protein |
| | ---------------------------------------------------------------------------------------- |
| | L V P G F G K A Y T I F N K T L M E A D A H Y K S V R |
| | ---------------------------------------------------------------------------------------- |
| 1201 | CTGGTGCCAG GGTTCGGGAA GGCTTATACT ATTTTCAACA AGACTCFTAT GGAGGCGGAT GCCCATTATA AGTCAGTTAG |
| | GACCACGGTC CCAAGCCCTT CCGAATATGA TAAAAGTTGT TCTGAGAATA CCTCCGCCTA CGGGTAATAT TCAGTCAATC |
| | Rabies-G protein |
| | --------------------- |
| | T W N E I I P • |
| | --------------------- |
| | GACTTGGAAT GAGATAATTC |
| | CTGAACCTTA CTCTATTAAG |
| | Rabies-G protein |
| | ---------------------------------------------------------------------------------------- |
| | • S K G C L R V G G R C H P H V N G V F F N G I I L G |
| | ---------------------------------------------------------------------------------------- |
| 1301 | CCTCCAAAGG ATGTCTGAGA GTCGGTGGGA GATGCCACCC CCATGTCAAT GGGGTGTTCT TTAACGGAAT CATCCFGGGA |
| | GGAGGTTTCC TACAGACTCT CAGCCACCCT CTACGGTGGG GGTACAGTTA CCCCACAAGA AATTGCCTTA GTAGGACCCT |
| | Rabies-G protein |
| | --------------------- |
| | P D G N V L I • |
| | --------------------- |
| | CCTGACGGGA ACGTGCTGAT |
| | GGACTGCCCT TGCACGACTA |
| | Rabies-G protein |
| | ---------------------------------------------------------------------------------------- |
| | • P E M Q S S L L Q Q H M E L L V S S V I P L M H P L A |
| | ---------------------------------------------------------------------------------------- |
| 1401 | TCCCGAGATG CAATCTTCCC TTCTGCAGCA ACACATGGAA CTCCTGGTGT CTTCAGTGAT ACCCCTGATG CACCCACTGG |
| | AGGGCTCTAC GTTAGAAGGG AAGACGTCGT TGTGTACCTT GAGGACCACA GAAGTCACTA TGGGGACTAC GTGGGTGACC |
| | Rabies-G protein |
| | --------------------- |
| | D P S T V F |
| | --------------------- |
| | CCGACCCCAG CACTGTGTTC |
| | GGCTGGGGTC GTGACACAAG |
| | Rabies-G protein |
| | ---------------------------------------------------------------------------------------- |
| | K N G D E A E D F V E V H L P D V H E R I S G V D L G |
| | ---------------------------------------------------------------------------------------- |
| 1501 | AAAAATGGCG ATGAGGCCGA AGACTTTGTG GAAGTTCACC TGCCCGATGT ACACGAAAGG ATATCTGGAG TAGACCTGGG |
| | TTTTACCGC TACTCCGGCT TCTGAAACAC CTTCAAGTGG ACGGGCTACA TGTGCTTTCC TATAGACCTC ATCTGGACCC |
| | Rabies-G protein |
| | --------------------- |
| | L P N W G K Y • |
| | --------------------- |
| | CCTTCCTAAT TGGGGTAAGT |
| | GGAAGGATTA ACCCCATTCA |
| | Rabies-G protein |
| | ---------------------------------------------------------------------------------------- |
| | • V L L S A G A L T A L M L I I F L M T C W R R V N R |
| | ---------------------------------------------------------------------------------------- |
| 1601 | ACGTGCTCCT GAGTGCGGGT GCCTTGACCG CTTTGATGCT GATCATTTTT CTGATGACCT GCTGGCGGAG GGTGAATCGC |
| | TGCACGAGGA CTCACGCCCA CGGAACTGGC GAAACTACGA CTAGTAAAAA GACTACTGGA CGACCGCCTC CCACTTAGCG |
| | Rabies-G protein |
| | --------------------- |
| | S E P T Q H N • |
| | --------------------- |
| | TCCGAGCCGA CACAGCACAA |
| | AGGCTCGGCT GTGTCGTGTT |
| | Rabies-G protein |
| | ---------------------------------------------------------------------------------------- |
| | • L R G T G R E V S V T P Q S G K I I S S W E S Y K S G |
| | ---------------------------------------------------------------------------------------- |
| 1701 | TCTCAGAGGG ACAGGCCGGG AAGTAAGTGT GACTCCGCAA TCTGGCAAGA TTATTAGTAG TTGGGAGAGT TACAAGTCTG |
| | AGAGTCTCCC TGTCCGGCCC TTCATTCACA CTGAGGCGTT AGACCGTTCT AATAATCATC AACCCTCTCA ATGTTCAGAC |
| | FMDV 2A |
| | --- |
| | Rabies-G protein |
| | ------------------ |
| | G E T G L N |
| | --------------------- |
| | GAGGAGAGAC TGGGTTGAAT |
| | CTCCTCTCTG ACCCAACTTA |
| | C/prM singal |
| | ------------------------------------ |
| | FMDV 2A |
| | ---------------------------------------------------- |
| | F D L L K L A G D V E S N P G P G G K T G I A V M I G |
| | ---------------------------------------------------------------------------------------- |
| 1801 | TTTGATCTGC TCAAACTTGC AGGCGATGTA GAATCAAATC CTGGACCCGG AGGAAAGACC GGTATTGCAG TCATGATTGG |
| | AAACTAGACG AGTTTGAACG TCCGCTACAT CTTAGTTTAG GACCTGGGCC TCCTTTCTGG CCATAACGTC AGTACTAACC |
| | C/prM signal |
| | --------------------- |
| | L I A C V G A • |
| | --------------------- |
| | CCTGATCGCC TGCGTAGGAG |
| | GGACTAGCGG ACGCATCCTC |
| | C/prM signal |
| | -- |
| | prM |
| | -------------------------------------------------------------------------------------- |
| | • V T L S N F Q G K V M M T V N A T D V T D V I T I P |
| | ---------------------------------------------------------------------------------------- |
| 1901 | CAGTTACCCT CTCTAACTTC CAAGGGAAGG TGATGATGAC GGTAAATGCT ACTGACGTCA CAGATGTCAT CACGATTCCA |
| | GTCAATGGGA GAGATTGAAG GTTCCCTTCC ACTACTACTG CCATTTACGA TGACTGCAGT GTCTACAGTA GTGCTAAGGT |
| | prM |
| | --------------------- |
| | T A A G K N L • |
| | --------------------- |
| | ACAGCTGCTG GAAAGAACCT |
| | TGTCGACGAC CTTTCTTGGA |
| | prM |
| | ---------------------------------------------------------------------------------------- |
| | • C I V R A M D V G Y M C D D T I T Y E C P V L S A G N |
| | ---------------------------------------------------------------------------------------- |
| 2001 | ATGCATTGTC AGAGCAATGG ATGTGGGATA CATGTGCGAT GATACTATCA CTTATGAATG CCCAGTGCTG TCGGCTGGTA |
| | TACGTAACAG TCTCGTTACC TACACCCTAT GTACACGCTA CTATGATAGT GAATACTTAC GGGTCACGAC AGCCGACCAT |
| | prM |
| | --------------------- |
| | D P E D I D |
| | --------------------- |
| | ATGATCCAGA AGACATCGAC |
| | TACTAGGTCT TCTGTAGCTG |
| | prM |
| | ---------------------------------------------------------------------------------------- |
| | C W C T K S A V Y V R Y G R C T K T R H S R R S R R S |
| | ---------------------------------------------------------------------------------------- |
| 2101 | TGTTGGTGCA CAAAGTCAGC AGTCTACGTC AGGTATGGAA GATGCACCAA GACACGCCAC TCAAGACGCA GTCGGAGGTC |
| | ACAACCACGT GTTTCAGTCG TCAGATGCAG TCCATACCTT CTACGTGGTT CTGTGCGGTG AGTTCTGCGT CAGCCTCCAG |
| | prM |
| | --------------------- |
| | L T V Q T H G • |
| | --------------------- |
| | ACTGACAGTG CAGACACACG |
| | TGACTGTCAC GTCTGTGTGC |
| | prM |
| | ---------------------------------------------------------------------------------------- |
| | • E S T L A N K K G A W M D S T K A T R Y L V K T E S |
| | ---------------------------------------------------------------------------------------- |
| 2201 | GAGAAAGCAC TCTAGCGAAC AAGAAGGGGG CTTGGATGGA CAGCACCAAG GCCACAAGGT ATTTGGTAAA AACAGAATCA |
| | CTCTTTCGTG AGATCGCTTG TTCTTCCCCC GAACCTACCT GTCGTGGTTC CGGTGTTCCA TAAACCATTT TTGTCTTAGT |
| | prM |
| | --------------------- |
| | W I L R N P G • |
| | --------------------- |
| | TGGATCTTGA GGAACCCTGG |
| | ACCTAGAACT CCTTGGGACC |
| | prM |
| | ---------------------------------------------------------------------------------------- |
| | • Y A L V A A V I G W M L G S N T M Q R V V F V V L L L |
| | ---------------------------------------------------------------------------------------- |
| 2301 | ATATGCCCTG GTGGCAGCCG TCATTGGTTG GATGCTTGGG AGCAACACCA TGCAGAGAGT TGTGTTTGTC GTGCTATTGC |
| | TATACGGGAC CACCGTCGGC AGTAACCAAC CTACGAACCC TCGTTGTGGT ACGTCTCTCA ACACAAACAG CACGATAACG |
| | prM |
| | --------------------- |
| | L V A P A Y |
| | --------------------- |
| | TTTTGGTGGC CCCAGCTTAC |
| | AAAACCACCG GGGTCGAATG |
| | E |
| | ------------------------------------------------------------------------------------- |
| | prM |
| | --- |
| | S F N C L G M S N R D F L E G V S G A T W V D L V L E |
| | ---------------------------------------------------------------------------------------- |
| 2401 | AGCTTTAACT GCCTTGGAAT GAGCAACAGA GACTTCTTGG AAGGAGTGTC TGGAGCAACA TGGGTGGATT TGGTTCTCGA |
| | TCGAAATTGA CGGAACCTTA CTCGTTGTCT CTGAAGAACC TTCCTCACAG ACCTCGTTGT ACCCACCTAA ACCAAGAGCT |
| | E |
| | --------------------- |
| | G D S C V T I • |
| | --------------------- |
| | AGGCGACAGC TGCGTGACTA |
| | TCCGCTGTCG ACGCACTGAT |
| | E |
| | ---------------------------------------------------------------------------------------- |
| | • M S K D K P T I D V K M M N M E A A N L A K V R S Y |
| | ---------------------------------------------------------------------------------------- |
| 2501 | TCATGTCTAA GGACAAGCCT ACCATCGATG TGAAGATGAT GAATATGGAG GCGGCCAACC TGGCAGAGGT CCGCAGTTAT |
| | AGTACAGATT CCTGTTCGGA TGGTAGCTAC ACTTCTACTA CTTATACCTC CGCCGGTTGG ACCGTCTCCA GGCGTCAATA |
| | E |
| | --------------------- |
| | C Y L A T V S • |
| | --------------------- |
| | TGCTATTTGG CTACCGTCAG |
| | ACGATAAACC GATGGCAGTC |
| | E |
| | ---------------------------------------------------------------------------------------- |
| | • D L S T K A A C P A M G E A H N D K R A D P A F V C R |
| | ---------------------------------------------------------------------------------------- |
| 2601 | CGATCTCTCC ACCAAAGCTG CGTGCCCGGC CATGGGAGAA GCTCACAATG ACAAACGTGC TGACCCAGCT TTTGTGTGCA |
| | GCTAGAGAGG TGGTTTCGAC GCACGGGCCG GTACCCTCTT CGAGTGTTAC TGTTTGCACG ACTGGGTCGA AAACACACGT |
| | E |
| | --------------------- |
| | Q G V V D R |
| | --------------------- |
| | GACAAGGAGT GGTGGACAGG |
| | CTGTTCCTCA CCACCTGTCC |
| | E |
| | ---------------------------------------------------------------------------------------- |
| | G W G N G C G L F G K G S I D T C A K F A C S T K A I |
| | ---------------------------------------------------------------------------------------- |
| 2701 | GGCTGGGGCA ACGGCTGCGG ACTATTTGGC AAAGGAAGCA TTGACACATG CGCCAAATTT GCCTGCTCTA CCAAGGCAAT |
| | CCGACCCCGT TGCCGACGCC TGATAAACCG TTTCCTTCGT AACTGTGTAC GCGGTTTAAA CGGACGAGAT GGTTCCGTTA |
| | E |
| | --------------------- |
| | G R T I L K E • |
| | --------------------- |
| | AGGAAGAACC ATTTTGAAAG |
| | TCCTTCTTGG TAAAACTTTC |
| | E |
| | ---------------------------------------------------------------------------------------- |
| | • N I K Y K V A I F V H G P T T V E S H G N Y S T Q V |
| | ---------------------------------------------------------------------------------------- |
| 2801 | AGAATATCAA GTACGAAGTG GCCATTTTTG TCCATGGACC AACTACTGTG GAGTCGCACG GAAACTACTC CACACAGGTT |
| | TCTTATAGTT CATGCTTCAC CGGTAAAAAC AGGTACCTGG TTGATGACAC CTCAGCGTGC CTTTGATGAG GTGTGTCCAA |
| | E |
| | --------------------- |
| | G A T Q A G R • |
| | --------------------- |
| | GGAGCCACTC AGGCAGGGAG |
| | CCTCGGTGAG TCCGTCCCTC |
| | E |
| | ---------------------------------------------------------------------------------------- |
| | • F S I T P A A P S Y T L K L G E Y G E V T V D C E P R |
| | ---------------------------------------------------------------------------------------- |
| 2901 | ATTCAGCATC ACTCCTGCGG CGCCTTCATA CACACTAAAG CTTGGAGAAT ATGGAGAGGT GACAGTGGAC TGTGAACCAC |
| | TAAGTCGTAG TGAGGACGCC GCGGAAGTAT GTGTGATTTC GAACCTCTTA TACCTCTCCA CTGTCACCTG ACACTTGGTG |
| | E |
| | --------------------- |
| | S G I D T N |
| | --------------------- |
| | GGTCAGGGAT TGACACCAAT |
| | CCAGTCCCTA ACTGTGGTTA |
| | E |
| | ---------------------------------------------------------------------------------------- |
| | A Y Y V M T V G T K T F L V H R E W F M D L N L P W S |
| | ---------------------------------------------------------------------------------------- |
| 3001 | GCATACTACG TGATGACTGT TGGAACAAAG ACGTTCTTGG TCCATCGTGA GTGGTTCATG GACCTCAACC TCCCTTGGAG |
| | CGTATGATGC ACTACTGACA ACCTTGTTTC TGCAAGAACC AGGTAGCACT CACCAAGTAC CTGGAGTTGG AGGGAACCTC |
| | E |
| | --------------------- |
| | S A G S T V W • |
| | --------------------- |
| | CAGTGCTGGA AGTACTGTGT |
| | GTCACGACCT TCATGACACA |
| | E |
| | ---------------------------------------------------------------------------------------- |
| | • R N R E T L M E F E K P H A T K Q S V I A L G S Q K |
| | ---------------------------------------------------------------------------------------- |
| 3101 | GGAGGAACAG AGAGACGTTA ATGGAGTTTG AGGAACCACA CGCCACGAAG CAGTCTGTGA TAGCATTGGG CTCACAAGAG |
| | CCTCCTTGTC TCTCTGCAAT TACCTCAAAC TCCTTGGTGT GCGGTGCTTC GTCAGACACT ATCGTAACCC GAGTGTTCTC |
| | E |
| | --------------------- |
| | G A L H Q A L • |
| | --------------------- |
| | GGAGCTCTGC ATCAAGCTTT |
| | CCTCGAGACG TAGTTCGAAA |
| | E |
| | ---------------------------------------------------------------------------------------- |
| | • A G A I P V E F S S N T V K L T S G H L K C R V K M E |
| | ---------------------------------------------------------------------------------------- |
| 3201 | GGCTGGAGCC ATTCCTGTGG AATTTTCAAG CAACACTGTC AAGTTGACGT CGGGTCATTT GAAGTGTAGA GTGAAGATGG |
| | CCGACCTCGG TAAGGACACC TTAAAAGTTC GTTGTGACAG TTCAACTGCA GCCCAGTAAA CTTCACATCT CACTTCTACC |
| | E |
| | --------------------- |
| | K L Q L K G |
| | --------------------- |
| | AAAAATTGCA GTTGAAGGGA |
| | TTTTTAACGT CAACTTCCCT |
| | E |
| | ---------------------------------------------------------------------------------------- |
| | T T Y G V C S K A F K F L G T P A D T G H G T V V L H |
| | ---------------------------------------------------------------------------------------- |
| 3301 | ACAACCTATG GCGTCTGTTC AAAGGCTTTC AAGTTTCTTG GGACTCCCGC AGACACAGGT CACGGCACTG TGGTGTTGGA |
| | TGTTGGATAC CGCAGACAAG TTTCCGAAAG TTCAAAGAAC CCTGAGGGCG TCTGTGTCCA GTGCCGTGAC ACCACAACCT |
| | E |
| | --------------------- |
| | L Q Y T G T D • |
| | --------------------- |
| | ATTGCAGTAC ACTGGCACGG |
| | TAACGTCATG TGACCGTGCC |
| | E |
| | ---------------------------------------------------------------------------------------- |
| | • G P C K V P J S S V A S L N D L T P V G R L V T V N |
| | ---------------------------------------------------------------------------------------- |
| 3401 | ATGGACCTTG CAAAGTTCCT ATCTCGTCAG TGGCTTCATT GAACGACCTA ACGCCAGTGG GCAGATTGGT CACTGTCAAC |
| | TACCTGGAAC GTTTCAAGGA TAGAGCAGTC ACCGAAGTAA CTTGCTGGAT TGCGGTCACC CGTCTAACCA GTGACAGTTG |
| | E |
| | --------------------- |
| | P F V S V A T • |
| | --------------------- |
| | CCTTTTGTTT CAGTGGCCAC |
| | GGAAAACAAA GTCACCGGTG |
| | E |
| | ---------------------------------------------------------------------------------------- |
| | • A N A K V L I E L E P P F G D S Y I V V G R G E Q Q I |
| | ---------------------------------------------------------------------------------------- |
| 3501 | GGCCAACGCT AAGGTCCTGA TTGAATTGGA ACCACCCTTT GGAGACTCAT ACATAGTGGT GGGCAGAGGA GAACAACAGA |
| | CCGGTTGCGA TTCCAGGACT AACTTAACCT TGGTGGGAAA CCTCTGAGTA TGTATCACCA CCCGTCTCCT CTTGTTGTCT |
| | E |
| | --------------------- |
| | N H H W H K |
| | --------------------- |
| | TCAATCACCA CTGGCACAAG |
| | AGTTAGTGGT GACCGTGTTC |
| | E |
| | ---------------------------------------------------------------------------------------- |
| | S G S S I G K A F T T T L K G A Q R L A A L G D T A W |
| | ---------------------------------------------------------------------------------------- |
| 3601 | TCTGGAAGCA GCATTGGCAA AGCCTTTACA ACCACCCTCA AAGGAGCGCA GAGACTAGCC GCTCTAGGAG ACACAGCTTG |
| | AGACCTTCGT CGTAACCGTT TCGGAAATGT TGGTGGGAGT TTCCTCGCGT CTCTGATCGG CGAGATCCTC TGTGTCGAAC |
| | E |
| | --------------------- |
| | D F G S V G G • |
| | --------------------- |
| | GGACTTTGGA TCAGTTGGAG |
| | CCTGAAACCT AGTCAACCTC |
| | E |
| | ---------------------------------------------------------------------------------------- |
| | • V F T S V G K A V H Q V F G G A F R S L F G G M S W |
| | ---------------------------------------------------------------------------------------- |
| 3701 | GGGTGTTCAC CTCAGTTGGG AAGGCTGTCC ATCAAGTGTT CGGAGGAGCA TTCCGCTCAC TGTTCGGAGG CATGTCCTGG |
| | CCCACAAGTG GAGTCAACCC TTCCGACAGG TAGTTCACAA GCCTCCTCGT AAGGCGAGTG ACAAGCCTCC GTACAGGACC |
| | E |
| | --------------------- |
| | I T Q G L L G • |
| | --------------------- |
| | ATAACGCAAG GATTGCTGGG |
| | TATTGCGTTC CTAACGACCC |
| | E |
| | ---------------------------------------------------------------------------------------- |
| | • A L L L W M G I N A R D R S I A L T F L A V G G V L L |
| | ---------------------------------------------------------------------------------------- |
| 3801 | GGCTCTCCTG TTGTGGATGG GCATCAATGC TCGTGACAGG TCCATAGCTC TCACGTTTCT CGCAGTTGGA GGAGTTCTGC |
| | CCGAGAGGAC AACACCTACC CGTAGTTACG AGCACTGTCC AGGTATCGAG AGTGCAAAGA GCGTCAACCT CCTCAAGACG |
| | E |
| | --------------------- |
| | F L S V N V |
| | --------------------- |
| | TCTTCCTCTC CGTGAACGTG |
| | AGAAGGAGAG GCACTTGCAC |
| | E |
| | ------ |
| | NS1 |
| | ---------------------------------------------------------------------------------- |
| | H A D T G C A I D I S R Q E L R C G S G V F I H N D V |
| | ---------------------------------------------------------------------------------------- |
| 3901 | CACGCTGACA CTGGGTGTGC CATAGACATC AGCCGGCAAG AGCTGAGATG TGGAAGTGGA GTGTTCATAC ACAATGATGT |
| | GTGCGACTGT GACCCACACG GTATCTGTAG TCCCCCGTTC TCGACTCTAC ACCTTCACCT CACAAGTATG TGTTACTACA |
| | NS1 |
| | --------------------- |
| | E A W M D R Y • |
| | --------------------- |
| | GGAGGCTTGG ATGGACCGGT |
| | CCTCCGAACC TACCTGGCCA |

WN (ΔprME)-Rabies G 20/45 sequence (partial)

| | |
|---|---|
| | 5′ UTR |
| | ---------------------------------------------------------------------------------------- |
| 1 | AGTAGTTCGC CTGTGTGAGC TGACAAACTT AGTAGTGTTT GTGAGGATTA ACAACAATTA ACACAGTGCG AGCTGTTTCT |
| | TCATCAAGCG GACACACTCG ACTGTTTGAA TCATCACAAA CACTCCTAAT TGTTGTTAAT TGTGTCACGC TCGACAAAGA |
| | C protein |
| | ---- |
| | 5′ UTR |
| | ----------------- |
| | M S • |
| | ---- |
| | TAGCACGAAG ATCTCGATGT |
| | ATCGTGCTTC TAGAGCTACA |
| | C protein |
| | ---------------------------------------------------------------------------------------- |
| | • K K P G G P G K S R A V Y L L K R G M P R V L S L I |
| | ---------------------------------------------------------------------------------------- |
| 101 | CTAAGAAACC AGGAGGGCCC GGCAAGAGCC GGGCTGTCTA TTTGCTAAAA CGCGGAATGC CCCGCGTGTT GTCCTTGATT |
| | GATTCTTTGG TCCTCCCGGG CCGTTCTCGG CCCGACAGAT AAACGATTTT GCGCCTTACG GGGCGCACAA CAGGAACTAA |
| | C protein |
| | --------------------- |
| | G L K R A M L • |
| | --------------------- |
| | GGACTTAAGA GGGCTATGTT |
| | CCTGAATTCT CCCGATACAA |
| | C protein |
| | ---------------------------------------------------------------------------------------- |
| | • S L I D G K G P I R F V L A L L A F F R F T A I A P T |
| | ---------------------------------------------------------------------------------------- |
| 201 | GAGCCTGATC GACGGCAAGG GGCCAATACG ATTTGTGTTG GCTCTCTTGG CGTTCTTCAG GTTCACAGCA ATTGCTCCGA |
| | CTCGGACTAG CTGCCGTTCC CCGGTTATGC TAAACACAAC CGAGAGAACC GCAAGAAGTC CAAGTGTCGT TAACGAGGCT |
| | C protein |
| | --------------------- |
| | R A V L D R |
| | --------------------- |
| | CCCGAGCAGT GCTGGATCGA |
| | GGGCTCGTCA CGACCTAGCT |
| | C protein |
| | ---------------------------------------------------------------------------------------- |
| | W R G V N K Q T A M K H L L S F K K E L G T L T S A I |
| | ---------------------------------------------------------------------------------------- |
| 301 | TGGAGAGGTG TGAACAAACA AACAGCGATG AAACACCTTC TGAGTTTCAA GAAGGAACTA GGGACCTTGA CCAGTGCTAT |
| | ACCTCTCCAC ACTTGTTTGT TTGTCGCTAC TTTGTGGAAG ACTCAAAGTT CTTCCTTGAT CCCTGGAACT GGTCACGATA |
| | C protein |
| | --------------------- |
| | N R R S S K Q • |
| | --------------------- |
| | CAATCGGCGG AGCTCAAAGC |
| | GTTAGCCGCC TCGAGTTTCG |
| | Rabies-G signal |
| | ----------------------------------------------- |
| | C protein partial C signal |
| | ------------ ---------------------------- |
| | • K K R G G K T G I A V I V P Q A L L F V P L L V F P |
| | ---------------------------------------------------------------------------------------- |
| 401 | AAAAGAAGCG AGGGGGCAAG ACTGGTATAG CTGTGATCGT TCCTCAGGCT CTTTTGTTTG TACCCTTGCT GGTATTTCCC |
| | TTTTCTTCGC TCCCCCGTTC TGACCATATC GACACTAGCA AGGAGTCCGA GAAAACAAAC ATGGGAACGA CCATAAAGGG |
| | Rabies-G signal |
| | ------------- |
| | RAbies-G protein |
| | -------- |
| | L C F G K F P • |
| | --------------------- |
| | CTTTGCTTTG GTAAATTTCC |
| | GAAACGAAAC CATTTAAAGG |
| | RAbies-G protein |
| | ---------------------------------------------------------------------------------------- |
| | • I Y T I P D K L G P W S P I D I H H L S C P N N L V V |
| | ---------------------------------------------------------------------------------------- |
| 501 | TATCTATACC ATCCCTGATA AGCTCGGGCC TTGGAGTCCC ATTGATATTC ACCATTTGAG CTGCCCAAAC AACCTCGTCG |
| | ATAGATATGG TAGGGACTAT TCGAGCCCGG AACCTCAGGG TAACTATAAG TGGTAAACTC GACGGGTTTG TTGGAGCAGC |
| | RAbies-G protein |
| | --------------------- |
| | E D P G C T |
| | --------------------- |
| | TTGAGGATGA AGGGTGCACT |
| | AACTCCTACT TCCCACGTGA |
| | RAbies-G protein |
| | ---------------------------------------------------------------------------------------- |
| | N L S G F S Y M E L K V G Y I S A I K M N G F T C T G |
| | ---------------------------------------------------------------------------------------- |
| 601 | AATCTTTCTG GATTTTCCTA CATGGAGTTG AAAGTGGGCT ATATTTCAGC CATTAAGATG AACGGCTTTA CTTGTACAGG |
| | TTAGAAAGAC CTAAAAGGAT GTACCTCAAC TTTCACCCGA TATAAAGTCG GTAATTCTAC TTGCCGAAAT GAACATGTCC |
| | RAbies-G protein |
| | --------------------- |
| | V V T E A E T • |
| | --------------------- |
| | AGTCGTGACC GAAGCCGAGA |
| | TCAGCACTGG CTTCGGCTCT |
| | RAbies-G protein |
| | ---------------------------------------------------------------------------------------- |
| | • Y T N F V G Y V T T T F K R K H F R P T P D A C R A |
| | ---------------------------------------------------------------------------------------- |
| 701 | CATATACAAA TTTCGTGGGA TACGTCACCA CCACCTTCAA GAGAAAACAC TTCCGCCCAA CGCCTGACGC TTGTCGGGCC |
| | GTATATGTTT AAAGCACCCT ATGCAGTGGT GGTGGAAGTT CTCTTTTGTG AAGGCGGGTT GCGGACTGCG AACAGCCCGG |
| | RAbies-G protein |
| | --------------------- |
| | A Y N W K M A • |
| | --------------------- |
| | GCTTACAACT GGAAGATGGC |
| | CGAATGTTGA CCTTCTACCG |
| | RAbies-G protein |
| | ---------------------------------------------------------------------------------------- |
| | • G D P R Y E E S L H N P Y P D Y H W L R T V K T T K E |
| | ---------------------------------------------------------------------------------------- |
| 801 | AGGAGATCCT CGATATGAAG AATCTCTGCA CAACCCGTAT CCTGATTACC ATTGGCTGCG GACAGTCAAG ACTACCAAGG |
| | TCCTCTAGGA GCTATACTTC TTAGAGACGT GTTGGGCATA GGACTAATGG TAACCGACGC CTGTCAGTTC TGATGGTTCC |
| | RAbies-G protein |
| | --------------------- |
| | S L V I I S |
| | --------------------- |
| | AGAGTCTGGT CATTATATCA |
| | TCTCAGACCA GTAATATAGT |
| | RAbies-G protein |
| | ---------------------------------------------------------------------------------------- |
| | P S V A D L D P Y D R S L H S R V F P G G N C S G V A |
| | ---------------------------------------------------------------------------------------- |
| 901 | CCAAGCGTGG CCGATCTTGA TCCTTATGAT AGATCCCTGC ACAGTAGGGT TTTTCCTGGC GGGAATTGTA GCGGTGTTGC |
| | GGTTCGCACC GGCTAGAACT AGGAATACTA TCTAGGGACG TGTCATCCCA AAAAGGACCG CCCTTAACAT CGCCACAACG |
| | RAbies-G protein |
| | --------------------- |
| | V S S T Y C S • |
| | --------------------- |
| | AGTATCAAGT ACCTACTGCT |
| | TCATAGTTCA TGGATGACGA |
| | RAbies-G protein |
| | ---------------------------------------------------------------------------------------- |
| | • T N H D Y T I W M P E N P R L G M S C D I F T N S R |
| | ---------------------------------------------------------------------------------------- |
| 1001 | CCACTAACCA CGACTACACT ATATGGATGC CTGAGAACCC TCGACTCGGT ATGAGTTGCG ACATTTTTAC GAACTCACGG |
| | GGTGATTGGT GCTGATGTGA TATACCTACG GACTCTTGGG AGCTGAGCCA TACTCAACGC TGTAAAAATG CTTGAGTGCC |
| | RAbies-G protein |
| | --------------------- |
| | G K R A S K G • |
| | --------------------- |
| | GGCAAGCGGG CATCTAAGGG |
| | CCGTTCGCCC GTAGATTCCC |
| | RAbies-G protein |
| | ---------------------------------------------------------------------------------------- |
| | • S E T C G F V D E R G L Y K S L K G A C K L K L C G V |
| | ---------------------------------------------------------------------------------------- |
| 1101 | GTCTGAAACA TGCGGGTTTG TTGATGAGCG GGGGTTGTAT AAATCTCTTA AAGGCGCCTG TAAGCTGAAA CTCTGTGGCG |
| | CAGACTTTGT ACGCCCAAAC AACTACTCGC CCCCAACATA TTTAGAGAAT TTCCGCGGAC ATTCGACTTT GAGACACCGC |
| | RAbies-G protein |
| | --------------------- |
| | L G L R L M |
| | --------------------- |
| | TACTGGGGCT GCGCCTGATG |
| | ATGACCCCGA CGCGGACTAC |
| | RAbies-G protein |
| | ---------------------------------------------------------------------------------------- |
| | D G T W V A M Q T S N E T K W C P P G Q L V N L H D F |
| | ---------------------------------------------------------------------------------------- |
| 1201 | GACGGCACAT GGGTGGCTAT GCAGACAAGC AATGAAACAA AGTGGTGTCC CCCTGGTCAG CTGGTTAATC TGCACGACTT |
| | CTGCCGTGTA CCCACCGATA CGTCTGTTCG TTACTTTGTT TCACCACAGG GGGACCAGTC GACCAATTAG ACGTGCTGAA |
| | RAbies-G protein |
| | --------------------- |
| | R S D E I E H • |
| | --------------------- |
| | TAGGTCTGAC GAAATCGAGC |
| | ATCCAGACTG CTTTAGCTCG |
| | RAbies-G protein |
| | ---------------------------------------------------------------------------------------- |
| | • L V V E E L V K K R E E C L D A L E S I M T T K S V |
| | ---------------------------------------------------------------------------------------- |
| 1301 | ACCTTGTGGT GGAGGAACTG GTGAAGAAAC GCGAAGAGTG CCTGGACGCA CTTGAGAGTA TTATGACCAC CAAATCCGTT |
| | TGGAACACCA CCTCCTTGAC CACTTCTTTG CGCTTCTCAC GGACCTGCGT GAACTCTCAT AATACTGGTG GTTTAGGCAA |
| | RAbies-G protein |
| | --------------------- |
| | S F R R L S H • |
| | --------------------- |
| | TCCTTCAGAA GACTGAGCCA |
| | AGGAAGTCTT CTGACTCGGT |
| | RAbies-G protein |
| | ---------------------------------------------------------------------------------------- |
| | • L R K L V P G F G K A Y T I F N K T L M E A D A H Y K |
| | ---------------------------------------------------------------------------------------- |
| 1401 | CCTGCGAAAG CTGGTGCCAG GGTTCGGGAA GGCTTATACT ATTTTCAACA AGACTCTTAT GGAGGCGGAT GCCCATTATA |
| | GGACGCTTTC GACCACGGTC CCAAGCCCTT CCGAATATGA TAAAAGTTGT TCTGAGAATA CCTCCGCCTA CGGGTAATAT |
| | RAbies-G protein |
| | --------------------- |
| | S V R T W N |
| | --------------------- |
| | AGTCAGTTAG GACTTGGAAT |
| | TCAGTCAATC CTGAACCTTA |
| | RAbies-G protein |
| | ---------------------------------------------------------------------------------------- |
| | E I I P S K G C L R V G G R C H P H V N G V F F N G I |
| | ---------------------------------------------------------------------------------------- |
| 1501 | GAGATAATTC CCTCCAAAGG ATGTCTGAGA GTCGGTGGGA GATGCCACCC CCATGTCAAT GGGGTGTTCT TTAACGGAAT |
| | CTCTATTAAG GGAGGTTTCC TACAGACTCT CAGCCACCCT CTACGGTGGG GGTACAGTTA CCCCACAAGA AATTGCCTTA |
| | RAbies-G protein |
| | --------------------- |
| | I L G P D G N • |
| | --------------------- |
| | CATCCTGGGA CCTGACGGGA |
| | GTAGGACCCT GGACTGCCCT |
| | RAbies-G protein |
| | ---------------------------------------------------------------------------------------- |
| | • V L I P E M Q S S L L Q Q H M E L L V S S V I P L M |
| | ---------------------------------------------------------------------------------------- |
| 1601 | ACGTGCTGAT TCCCGAGATG CAATCTTCCC TTCTGCAGCA ACACATGGAA CTCCTGGTGT CTTCAGTGAT ACCCCTGATG |
| | TGCACGACTA AGGGCTCTAC GTTAGAAGGG AAGACGTCGT TGTGTACCTT GAGGACCACA GAAGTCACTA TGGGGACTAC |
| | RAbies-G protein |
| | --------------------- |
| | H P L A D P S • |
| | --------------------- |
| | CACCCACTGG CCGACCCCAG |
| | GTGGGTGACC GGCTGGGGTC |
| | RAbies-G protein |
| | ---------------------------------------------------------------------------------------- |
| | • T V F K N G D E A E D F V E V H L P D V H E R I S G V |
| | ---------------------------------------------------------------------------------------- |
| 1701 | CACTGTGTTC AAAAATGGCG ATGAGGCCGA AGACTTTGTG GAAGTTCACC TGCCCGATGT ACACGAAAGG ATATCTGGAG |
| | GTGACACAAG TTTTTACCGC TACTCCGGCT TCTGAAACAC CTTCAAGTGG ACGGGCTACA TGTGCTTTCC TATAGACCTC |
| | RAbies-G protein |
| | --------------------- |
| | D L G L P N |
| | --------------------- |
| | TAGACCTGGG CCTTCCTAAT |
| | ATCTGGACCC GGAAGGATTA |
| | RAbies-G protein |
| | ---------------------------------------------------------------------------------------- |
| | W G K Y V L L S A G A L T A L M L I I F L M T C W R R |
| | ---------------------------------------------------------------------------------------- |
| 1801 | TGGGGTAAGT ACGTGCTCCT GAGTGCGGGT GCCTTGACCG CTTTGATGCT GATCATTTTT CTGATGACCT GCTGGCGGAG |
| | ACCCCATTCA TGCACGAGGA CTCACGCCCA CGGAACTGGC GAAACTACGA CTAGTAAAAA GACTACTGGA CGACCGCCTC |
| | RAbies-G protein |
| | --------------------- |
| | V N R S E P T • |
| | --------------------- |
| | GGTGAATCGC TCCGAGCCGA |
| | CCACTTAGCG AGGCTCGGCT |
| | RAbies-G protein |
| | ---------------------------------------------------------------------------------------- |
| | • Q H N L R G T G R E V S V T P Q S G K I I S S W E S |
| | ---------------------------------------------------------------------------------------- |
| 1901 | CACAGCACAA TCTCAGAGGG ACAGGCCGGG AAGTAAGTGT GACTCCGCAA TCTGGCAAGA TTATTAGTAG TTGGGAGAGT |
| | GTGTCGTGTT AGAGTCTCCC TGTCCGGCCC TTCATTCACA CTGAGGCGTT AGACCGTTCT AATAATCATC AACCCTCTCA |
| | RAbies-G protein |
| | --------------------- |
| | Y K S G G E T • |
| | --------------------- |
| | TACAAGTCTG GAGGAGAGAC |
| | ATGTTCAGAC CTCCTCTCTG |
| | FMDV 2A NS1 signal |
| | --------------------------------------------------------- --------------- |
| | RAbies-G protein preNS1 signal |
| | ------- ---------- |
| | • G L N F D L L K L A G D V E S N P G P A R D R S I A L |
| | ---------------------------------------------------------------------------------------- |
| 2001 | TGGGTTGAAT TTTGATCTGC TCAAACTTGC AGGCGATGTA GAATCAAATC CTGGACCCGC CCGGGACAGG TCCATAGCTC |
| | ACCCAACTTA AAACTAGACG AGTTTGAACG TCCGCTACAT CTTAGTTTAG GACCTGGGCG GGCCCTGTCC AGGTATCGAG |
| | NS1 signal |
| | --------------------- |
| | T F L A V G |
| | --------------------- |
| | TCACGTTTCT CGCAGTTGGA |
| | AGTGCAAAGA GCGTCAACCT |
| | NS1 |
| | ------------------------------------------------- |
| | NS1 signal |
| | --------------------------------------- |
| | G V L L F L S V N V H A D T G C A I D I S R Q E L R C |
| | ---------------------------------------------------------------------------------------- |
| 2101 | GGAGTTCTGC TCTTCCTCTC CGTGAACGTG CACGCTGACA CTGGGTGTGC CATAGACATC AGCCGGCAAG AGCTGAGATG |
| | CCTCAAGACG AGAAGGAGAG GCACTTGCAC GTGCGACTGT GACCCACACG GTATCTGTAG TCGGCCGTTC TCGACTCTAC |
| | NS1 |
| | --------------------- |
| | G S G V F I H • |
| | --------------------- |
| | TGGAAGTGGA GTGTTCATAC |
| | ACCTTCACCT CACAAGTATG |

PIV-WNV helper ΔNS1

| | |
|---|---|
| | 5′ UTR |
| | ---------------------------------------------------------------------------------------- |
| 1 | AGTAGTTCGC CTGTGTGAGC TGACAAACTT AGTAGTGTTT GTGAGGATTA ACAACAATTA ACACAGTGCG AGCTGTTTCT |
| | TCATCAAGCG GACACACTCG ACTGTTTGAA TCATCACAAA CACTCCTAAT TGTTGTTAAT TGTGTCACGC TCGACAAAGA |
| | C |
| | ---- |
| | 5′ UTR |
| | ----------------- |
| | M S • |
| | TAGCACGAAG ATCTCGATGT |
| | ATCGTGCTTC TAGAGCTACA |
| | C |
| | ---------------------------------------------------------------------------------------- |
| | • K K P G G P G K S R A V N M L K R G M P R V L S L I |
| 101 | CTAAGAAACC AGGAGGGCCC GGCAAGAGCC GGGCTGTCAA TATGCTAAAA CGCGGAATGC CCCGCGTGTT GTCCTTGATT |
| | GATTCTTTGG TCCTCCCGGG CCGTTCTCGG CCCGACAGTT ATACGATTTT GCGCCTTACG GGGCGCACAA CAGGAACTAA |
| | C |
| | --------------------- |
| | G L K R A M L • |
| | GGACTTAAGA GGGCTATGTT |
| | CCTGAATTCT CCCGATACAA |
| | C |
| | ---------------------------------------------------------------------------------------- |
| | • S L I D G K G P I R F V L A L L A F F R F T A I A P T |
| 201 | GAGCCTGATC GACGGCAAGG GGCCAATACG ATTTGTGTTG GCTCTCTTGG CGTTCTTCAG GTTCACAGCA ATTGCTCCGA |
| | CTCGGACTAG CTGCCGTTCC CCGGTTATGC TAAACACAAC CGAGAGAACC GCAAGAAGTC CAAGTGTCGT TAACGAGGCT |
| | C |
| | --------------------- |
| | R A V L D R |
| | CCCGAGCAGT GCTGGATCGA |
| | GGGCTCGTCA CGACCTAGCT |
| | C |
| | ---------------------------------------------------------------------------------------- |
| | W R G V N K Q T A M K H L L S F K K E L G T L T S A I |
| 301 | TGGAGAGGTG TGAACAAACA AACAGCGATG AAACACCTTC TGAGTTTCAA GAAGGAACTA GGGACCTTGA CCAGTGCTAT |
| | ACCTCTCCAC ACTTGTTTGT TTGTCGCTAC TTTGTGGAAG ACTCAAAGTT CTTCCTTGAT CCCTGGAACT GGTCACGATA |
| | C |
| | --------------------- |
| | N R R S S K Q • |
| | CAATCGGCGG AGCTCAAAAC |
| | GTTAGCCGCC TCGAGTTTTG |
| | Signal peptide |
| | ----------------------------------------------------------- |
| | C prM |
| | ------------ ---------------- |
| | • K K R G G K T G I A V M I G L I A S V G A V T L S N |
| 401 | AAAAGAAAAG AGGAGGAAAG ACCGGAATTG CAGTCATGAT TGGCCTGATC GCCAGCGTAG GAGCAGTTAC CCTCTCTAAC |
| | TTTTCTTTTC TCCTCCTTTC TGGCCTTAAC GTCAGTACTA ACCGGACTAG CGGTCGCATC CTCGTCAATG GGAGAGATTG |
| | prM |
| | --------------------- |
| | F Q G K V M M • |
| | TTCCAAGGGA AGGTGATGAT |
| | AAGGTTCCCT TCCACTACTA |
| | prM |
| | ---------------------------------------------------------------------------------------- |
| | • T V N A T D V T D V I T I P T A A G K N L C I V R A M |
| 501 | GACGGTAAAT GCTACTGACG TCACAGATGT CATCACGATT CCAACAGCTG CTGGAAAGAA CCTATGCATT GTCAGAGCAA |
| | CTGCCATTTA CGATGACTGC AGTGTCTACA GTAGTGCTAA GGTTGTCGAC GACCTTTCTT GGATACGTAA CAGTCTCGTT |
| | prM |
| | --------------------- |
| | D V G Y M C |
| | TGGATGTGGG ATACATGTGC |
| | ACCTACACCC TATGTACACG |
| | prM |
| | ---------------------------------------------------------------------------------------- |
| | D D T I T Y E C P V L S A G N D P E D I D C W C T K S |
| 601 | GATGATACTA TCACTTATGA ATGCCCAGTG CTGTCGGCTG GTAATGATCC AGAAGACATC GACTGTTGGT GCACAAAGTC |
| | CTACTATGAT AGTGAATACT TACGGGTCAC GACAGCCGAC CATTACTAGG TCTTCTGTAG CTGACAACCA CGTGTTTCAG |
| | prM |
| | --------------------- |
| | A V Y V R Y G • |
| | AGCAGTCTAC GTCAGGTATG |
| | TCGTCAGATG CAGTCCATAC |
| | prM |
| | ---------------------------------------------------------------------------------------- |
| | • R C T K T R H S R R S R R S L T V Q T H G E S T L A |
| 701 | GAAGATGCAC CAAGACACGC CACTCAAGAC GCAGTCGGAG GTCACTGACA GTGCAGACAC ACGGAGAAAG CACTCTAGCG |
| | CTTCTACGTG GTTCTGTGCG GTGAGTTCTG CGTCAGCCTC CAGTGACTGT CACGTCTGTG TGCCTCTTTC GTGAGATCGC |
| | prM |
| | --------------------- |
| | N K K G A W M • |
| | AACAAGAAGG GGGCTTGGAT |
| | TTGTTCTTCC CCCGAACCTA |
| | prM |
| | ---------------------------------------------------------------------------------------- |
| | • D S T K A T R Y L V K T E S W I L R N P G Y A L V A A |
| 801 | GGACAGCACC AAGGCCACAA GGTATTTGGT AAAAACAGAA TCATGGATCT TGAGGAACCC TGGATATGCC CTGGTGGCAG |
| | CCTGTCGTGG TTCCGGTGTT CCATAAACCA TTTTTGTCTT AGTACCTAGA ACTCCTTGGG ACCTATACGG GACCACCGTC |
| | prM |
| | --------------------- |
| | V I G W M L |
| | CCGTCATTGG TTGGATGCTT |
| | GGCAGTAACC AACCTACGAA |
| | E |
| | ---------------- |
| | prM |
| | ------------------------------------------------------------------------ |
| | G S N T M Q R V V F V V L L L L V A P A Y S F N C L G |
| 901 | GGGAGCAACA CCATGCAGAG AGTTGTGTTT GTCGTGCTAT TGCTTTTGGT GGCCCCAGCT TACAGCTTTA ACTGCCTTGG |
| | CCCTCGTTGT GGTACGTCTC TCAACACAAA CAGCACGATA ACGAAAACCA CCGGGGTCGA ATGTCGAAAT TGACGGAACC |
| | E |
| | --------------------- |
| | M S N R D F L • |
| | AATGAGCAAC AGAGACTTCT |
| | TTACTCGTTG TCTCTGAAGA |
| | E |
| | ---------------------------------------------------------------------------------------- |
| | • E G V S G A T W V D L V L E G D S C V T I M S K D K |
| 1001 | TGGAAGGAGT GTCTGGAGCA ACATGGGTGG ATTTGGTTCT CGAAGGCGAC AGCTGCGTGA CTATCATGTC TAAGGACAAG |
| | ACCTTCCTCA CAGACCTCGT TGTACCCACC TAAACCAAGA GCTTCCGCTG TCGACGCACT GATAGTACAG ATTCCTGTTC |
| | E |
| | --------------------- |
| | P T I D V K M • |
| | CCTACCATCG ATGTGAAGAT |
| | GGATGGTAGC TACACTTCTA |
| | E |
| | ---------------------------------------------------------------------------------------- |
| | • M N M E A A N L A E V R S Y C Y L A T V S D L S T K A |
| 1101 | GATGAATATG GAGGCGGCCA ACCTGGCAGA GGTCCGCAGT TATTGCTATT TGGCTACCGT CAGCGATCTC TCCACCAAAG |
| | CTACTTATAC CTCCGCCGGT TGGACCGTCT CCAGGCGTCA ATAACGATAA ACCGATGGCA GTCGCTAGAG AGGTGGTTTC |
| | E |
| | --------------------- |
| | A C P A M G |
| | CTGCGTGCCC GGCCATGGGA |
| | GACGCACGGG CCGGTACCCT |
| | E |
| | ---------------------------------------------------------------------------------------- |
| | E A H N D K R A D P A F V C R Q G V V D R G W G N G C |
| 1201 | GAAGCTCACA ATGACAAACG TGCTGACCCA GCTTTTGTGT GCAGACAAGG AGTGGTGGAC AGGGGCTGGG GCAACGGCTG |
| | CTTCGAGTGT TACTGTTTGC ACGACTGGGT CGAAAACACA CGTCTGTTCC TCACCACCTG TCCCCGACCC CGTTGCCGAC |
| | E |
| | --------------------- |
| | G L F G K G S • |
| | CGGACTATTT GGCAAAGGAA |
| | GCCTGATAAA CCGTTTCCTT |
| | E |
| | ---------------------------------------------------------------------------------------- |
| | • I D T C A K F A C S T K A I G R T I L K E N I K Y E |
| 1301 | GCATTGACAC ATGCGCCAAA TTTGCCTGCT CTACCAAGGC AATAGGAAGA ACCATTTTGA AAGAGAATAT CAAGTACGAA |
| | CGTAACTGTG TACGCGGTTT AAACGGACGA GATGGTTCCG TTATCCTTCT TGGTAAAACT TTCTCTTATA GTTCATGCTT |
| | E |
| | --------------------- |
| | V A I F V H G • |
| | GTGGCCATTT TTGTCCATGG |
| | CACCGGTAAA AACAGGTACC |
| | E |
| | ---------------------------------------------------------------------------------------- |
| | • P T T V E S H G N Y S T Q V G A T Q A G R F S I T P A |
| 1401 | ACCAACTACT GTGGAGTCGC ACGGAAACTA CTCCACACAG GTTGGAGCCA CTCAGGCAGG GAGATTCAGC ATCACTCCTG |
| | TGGTTGATGA CACCTCAGCG TGCCTTTGAT GAGGTGTGTC CAACCTCGGT GAGTCCGTCC CTCTAAGTCG TAGTGAGGAC |
| | E |
| | --------------------- |
| | A P S Y T L |
| | CGGCGCCTTC ATACACACTA |
| | GCCGCGGAAG TATGTGTGAT |
| | E |
| | ---------------------------------------------------------------------------------------- |
| | K L G E Y G E V T V D C E P R S G I D T N A Y Y V M T |
| 1501 | AAGCTTGGAG AATATGGAGA GGTGACAGTG GACTGTGAAC CACGGTCAGG GATTGACACC AATGCATACT ACGTGATGAC |
| | TTCGAACCTC TTATACCTCT CCACTGTCAC CTGACACTTG GTGCCAGTCC CTAACTGTGG TTACGTATGA TGCACTACTG |
| | E |
| | --------------------- |
| | V G T K T F L • |
| | TGTTGGAACA AAGACGTTCT |
| | ACAACCTTGT TTCTGCAAGA |
| | E |
| | ---------------------------------------------------------------------------------------- |
| | • V H R E W F M D L N L P W S S A G S T V W R N R E T |
| 1601 | TGGTCCATCG TGAGTGGTTC ATGGACCTCA ACCTCCCTTG GAGCAGTGCT GGAAGTACTG TGTGGAGGAA CAGAGAGACG |
| | ACCAGGTAGC ACTCACCAAG TACCTGGAGT TGGAGGGAAC CTCGTCACGA CCTTCATGAC ACACCTCCTT GTCTCTCTGC |
| | E |
| | --------------------- |
| | L M E F E E P • |
| | TTAATGGAGT TTGAGGAACC |
| | AATTACCTCA AACTCCTTGG |
| | E |
| | ---------------------------------------------------------------------------------------- |
| | • H A T K Q S V I A L G S Q E G A L H Q A L A G A I P V |
| 1701 | ACACGCCACG AAGCAGTCTG TGATAGCATT GGGCTCACAA GAGGGAGCTC TGCATCAAGC TTTGGCTGGA GCCATTCCTG |
| | TGTGCGGTGC TTCGTCAGAC ACTATCGTAA CCCGAGTGTT CTCCCTCGAG ACGTAGTTCG AAACCGACCT CGGTAAGGAC |
| | E |
| | --------------------- |
| | E F S S N T |
| | TGGAATTTTC AAGCAACACT |
| | ACCTTAAAAG TTCGTTGTGA |
| | E |
| | ---------------------------------------------------------------------------------------- |
| | V K L T S G H L K C R V K M E K L Q L K G T T Y G V C |
| 1801 | GTCAAGTTGA CGTCGGGTCA TTTGAAGTGT AGAGTGAAGA TGGAAAAATT GCAGTTGAAG GGAACAACCT ATGGCGTCTG |
| | CAGTTCAACT GCAGCCCAGT AAACTTCACA TCTCACTTCT ACCTTTTTAA CGTCAACTTC CCTTGTTGGA TACCGCAGAC |
| | E |
| | --------------------- |
| | S K A F K F L • |
| | TTCAAAGGCT TTCAAGTTTC |
| | AAGTTTCCGA AAGTTCAAAG |
| | E |
| | ---------------------------------------------------------------------------------------- |
| | • G T P A D T G H G T V V L E L Q Y T G T D G P C K V |
| 1901 | TTGGGACTCC CGCAGACACA GGTCACGGCA CTGTGGTGTT GGAATTGCAG TACACTGGCA CGGATGGACC TTGCAAAGTT |
| | AACCCTGAGG GCGTCTGTGT CCAGTGCCGT GACACCACAA CCTTAACGTC ATGTGACCGT GCCTACCTGG AACGTTTCAA |
| | E |
| | --------------------- |
| | P I S S V A S • |
| | CCTATCTCGT CAGTGGCTTC |
| | GGATAGAGCA GTCACCGAAG |
| | E |
| | ---------------------------------------------------------------------------------------- |
| | • L N D L T P V G R L V T V N P F V S V A T A N A K V L |
| 2001 | ATTGAACGAC CTAACGCCAG TGGGCAGATT GGTCACTGTC AACCCTTTTG TTTCAGTGGC CACGGCCAAC GCTAAGGTCC |
| | TAACTTGCTG GATTGCGGTC ACCCGTCTAA CCAGTGACAG TTGGGAAAAC AAAGTCACCG GTGCCGGTTG CGATTCCAGG |
| | E |
| | --------------------- |
| | I E L E P P |
| | TGATTGAATT GGAACCACCC |
| | ACTAACTTAA CCTTGGTGGG |
| | E |
| | ---------------------------------------------------------------------------------------- |
| | F G D S Y I V V G R G E Q Q I N H H W H K S G S S I G |
| 2101 | TTTGGAGACT CATACATAGT GGTGGGCAGA GGAGAACAAC AGATCAATCA CCACTGGCAC AAGTCTGGAA GCAGCATTGG |
| | AAACCTCTGA GTATGTATCA CCACCCGTCT CCTCTTGTTG TCTAGTTAGT GGTGACCGTG TTCAGACCTT CGTCGTAACC |
| | E |
| | --------------------- |
| | K A F T T T L • |
| | CAAAGCCTTT ACAACCACCC |
| | GTTTCGGAAA TGTTGGTGGG |
| | E |
| | ---------------------------------------------------------------------------------------- |
| | • K G A Q R L A A L G D T A W D F G S V G G V F T S V |
| 2201 | TCAAAGGAGC GCAGAGACTA GCCGCTCTAG GAGACACAGC TTGGGACTTT GGATCAGTTG GAGGGGTGTT CACCTCAGTT |
| | AGTTTCCTCG CGTCTCTGAT CGGCGAGATC CTCTGTGTCG AACCCTGAAA CCTAGTCAAC CTCCCCACAA GTGGAGTCAA |
| | E |
| | --------------------- |
| | G K A V H Q V • |
| | GGGAAGGCTG TCCATCAAGT |
| | CCCTTCCGAC AGGTAGTTCA |
| | E |
| | ---------------------------------------------------------------------------------------- |
| | • F G G A F R S L F G G M S W I T Q G L L G A L L L W M |
| 2301 | GTTCGGAGGA GCATTCCGCT CACTGTTCGG AGGCATGTCC TGGATAACGC AAGGATTGCT GGGGGCTCTC CTGTTGTGGA |
| | CAAGCCTCCT CGTAAGGCGA GTGACAAGCC TCCGTACAGG ACCTATTGCG TTCCTAACGA CCCCCGAGAG GACAACACCT |
| | E |
| | --------------------- |
| | G I N A R D |
| | TGGGCATCAA TGCTCGTGAC |
| | ACCCGTAGTT ACGAGCACTG |
| | deleted |
| | NS1 |
| | ------------- |
| | E |
| | --------------------------------------------------------------------------- |
| | R S I A L T F L A V G G V L L F L S V N V H A D T G I |
| 2401 | AGGTCCATAG CTCTCACGTT TCTCGCAGTT GGAGGAGTTC TGCTCTTCCT CTCCGTGAAC GTGCACGCTG ACACTGGGAT |
| | TCCAGGTATC GAGAGTGCAA AGAGCGTCAA CCTCCTCAAG ACGAGAAGGA GAGGCACTTG CACGTGCGAC TGTGACCCTA |
| | deleted NS1 |
| | --------------------- |
| | H R G P A T R • |
| | CCACCGTGGA CCTGCCACTC |
| | GGTGGCACCT GGACGGTGAG |
| | deleted NS1 |
| | ---------------------------------------------------------------------------------------- |
| | • T T T E S G K L I T D W C C R S C T L P P L R Y Q T |
| 2501 | GCACCACCAC AGAGAGCGGA AAGTTGATAA CAGATTGGTG CTGCAGGAGC TGCACCTTAC CACCACTGCG CTACCAAACT |
| | CGTGGTGGTG TCTCTCGCCT TTCAACTATT GTCTAACCAC GACGTCCTCG ACGTGGAATG GTGGTGACGC GATGGTTTGA |
| | deleted NS1 |
| | --------------------- |
| | D S G C W Y G • |
| | GACAGCGGCT GTTGGTATGG |
| | CTGTCGCCGA CAACCATACC |
| | deleted NS1 |
| | ------------------------------------------------------------------- |
| | NS2A |
| | -------------------- |
| | • M E I R P Q R H D E K T L V Q S Q V N A Y N A D M I D |
| 2601 | TATGGAGATC AGACCACAGA GACATGATGA AAAGACCCTC GTGCAGTCAC AAGTGAATGC TTATAATGCT GATATGATTG |
| | ATACCTCTAG TCTGGTGTCT CTGTACTACT TTTCTGGGAG CACGTCAGTG TTCACTTACG AATATTACGA CTATACTAAC |
| | NS2A |
| | --------------------- |
| | P F Q L G L |
| | ACCCTTTTCA GTTGGGCCTT |
| | TGGGAAAAGT CAACCCGGAA |

SEQUENCE APPENDIX 5
PIV-WNV(ΔprME)/RSV-F

| | |
|---|---|
| | 5′ UTR |
| | ---------------------------------------------------------------------------------------- |
| 1 | AGTAGTTCGC CTGTGTGAGC TGACAAACTT AGTAGTGTTT GTGAGGATTA ACAACAATTA ACACAGTGCG AGCTGTTTCT |
| | TCATCAAGCG GACACACTCG ACTGTTTGAA TCATCACAAA CACTCCTAAT TGTTGTTAAT TGTGTCACGC TCGACAAAGA |
| | C protein |
| | ---- |
| | 5′ UTR |
| | ----------------- |
| | M S • |
| | TAGCACGAAG ATCTCGATGT |
| | ATCGTGCTTC TAGAGCTACA |
| | C protein |
| | ---------------------------------------------------------------------------------------- |
| | • K K P G G P G K S R A V Y L L K R G M P R V L S L I |
| 101 | CTAAGAAACC AGGAGGGCCC GGCAAGAGCC GGGCTGTCTA TTTGCTAAAA CGCGGAATGC CCCGCGTGTT GTCCTTGATT |
| | GATTCTTTGG TCCTCCCGGG CCGTTCTCGG CCCGACAGAT AAACGATTTT GCGCCTTACG GGGCGCACAA CAGGAACTAA |
| | C protein |
| | --------------------- |
| | G L K R A M L • |
| | GGACTTAAGA GGGCTATGTT |
| | CCTGAATTCT CCCGATACAA |
| | C protein |
| | ---------------------------------------------------------------------------------------- |
| | • S L I D G K G P I R F V L A L L A F F R F T A I A P T |
| 201 | GAGCCTGATC GACGGCAAGG GGCCAATACG ATTTGTGTTG GCTCTCTTGG CGTTCTTCAG GTTCACAGCA ATTGCTCCGA |
| | CTCGGACTAG CTGCCGTTCC CCGGTTATGC TAAACACAAC CGAGAGAACC GCAAGAAGTC CAAGTGTCGT TAACGAGGCT |
| | C protein |
| | --------------------- |
| | R A V L D R |
| | CCCGAGCAGT GCTGGATCGA |
| | GGGCTCGTCA CGACCTAGCT |
| | C protein |
| | ---------------------------------------------------------------------------------------- |
| | W R G V N K Q T A M K H L L S F K K E L G T L T S A I |
| 301 | TGGAGAGGTG TGAACAAACA AACAGCGATG AAACACCTTC TGAGTTTCAA GAAGGAACTA GGGACCTTGA CCAGTGCTAT |
| | ACCTCTCCAC ACTTGTTTGT TTGTCGCTAC TTTGTGGAAG ACTCAAAGTT CTTCCTTGAT CCCTGGAACT GGTCACGATA |
| | NS3 cleavage |
| | ---- |
| | C protein |
| | ----------------- |
| | N R R S S K Q • |
| | CAATCGGCGG AGCTCAAAGC |
| | GTTAGCCGCC TCGAGTTTCG |
| | F signal |
| | ----------------------------------------------------------------------- |
| | NS3 cleavage |
| | ------------ |
| | • K K R G G E L L I L K A N A I T T I L T A V T F C F |
| 401 | AAAAGAAGCG AGGGGGCGAG TTGCTAATCC TCAAAGCAAA TGCAATTACC ACAATCCTCA CTGCAGTCAC ATTTTGTTTT |
| | TTTTCTTCGC TCCCCCGCTC AACGATTAGG AGTTTCGTTT ACGTTAATGG TGTTAGGAGT GACGTCAGTG TAAAACAAAA |
| | F1 |
| | --------------------- |
| | A S G Q N I T • |
| | GCTTCTGGTC AAAACATCAC |
| | CGAAGACCAG TTTTGTAGTG |
| | F1 |
| | ---------------------------------------------------------------------------------------- |
| | • E E F Y Q S T C S A V S K G Y L S A L R T G W Y T S V |
| 501 | TGAAGAATTT TATCAATCAA CATGCAGTGC AGTTAGCAAA GGCTATCTTA GTGCTCTGAG AACTGGTTGG TATACCAGTG |
| | ACTTCTTAAA ATAGTTAGTT GTACGTCACG TCAATCGTTT CCGATAGAAT CACGAGACTC TTGACCAACC ATATGGTCAC |
| | F1 |
| | --------------------- |
| | I T I E L S |
| | TTATAACTAT AGAATTAAGT |
| | AATATTGATA TCTTAATTCA |
| | F1 |
| | ---------------------------------------------------------------------------------------- |
| | N I K E N K C N G T D A K V K L I K Q E L D K Y K N A |
| 601 | AATATCAAGG AAAATAAGTG TAATGGAACA GATGCTAAGG TAAAATTGAT AAAACAAGAA TTAGATAAAT ATAAAAATGC |
| | TTATAGTTCC TTTTATTCAC ATTACCTTGT CTACGATTCC ATTTTAACTA TTTTGTTCTT AATCTATTTA TATTTTTACG |
| | F1 |
| | --------------------- |
| | V T E L Q L L • |
| | TGTAACAGAA TTGCAGTTGC |
| | ACATTGTCTT AACGTCAACG |
| | F1 |
| | ---------------------------------------------------------------------------------------- |
| | • M Q S T P P T N N R A R R E L P R F M N Y T L N N A |
| 701 | TCATGCAAAG CACACCACCA ACAAACAATC GAGCCAGAAG AGAACTACCA AGGTTTATGA ATTATACACT CAACAATGCC |
| | AGTACGTTTC GTGTGGTGGT TGTTTGTTAG CTCGGTCTTC TCTTGATGGT TCCAAATACT TAATATGTGA GTTGTTACGG |
| | F1 |
| | --------------------- |
| | K K T N V T L • |
| | AAAAAAACCA ATGTAACATT |
| | TTTTTTTGGT TACATTGTAA |
| | F1 |
| | ------------------------ |
| | F2 |
| | ---------------------------------------------------------------- |
| | • S K K R K R R F L G F L L G V G S A I A S G V A V S K |
| 801 | AAGCAAGAAA AGGAAAAGAA GATTTCTTGG TTTTTTGTTA GGTGTTGGAT CTGCAATCGC CAGTGGCGTT GCTGTATCTA |
| | TTCGTTCTTT TCCTTTTCTT CTAAAGAACC AAAAAACAAT CCACAACCTA GACGTTAGCG GTCACCGCAA CGACATAGAT |
| | F2 |
| | --------------------- |
| | V L H L E G |
| | AGGTCCTGCA CCTAGAAGGG |
| | TCCAGGACGT GGATCTTCCC |
| | F2 |
| | ---------------------------------------------------------------------------------------- |
| | E V N K I K S A L L S T N K A V V S L S N G V S V L T |
| 901 | GAAGTGAACA AGATCAAAAG TGCTCTACTA TCCACAAACA AGGCTGTAGT CAGCTTATCA AATGGAGTTA GTGTCTTAAC |
| | CTTCACTTGT TCTAGTTTTC ACGAGATGAT AGGTGTTTGT TCCGACATCA GTCGAATAGT TTACCTCAAT CACAGAATTG |
| | F2 |
| | --------------------- |
| | S K V L D L K • |
| | CAGCAAAGTG TTAGACCTCA |
| | GTCGTTTCAC AATCTGGAGT |
| | F2 |
| | ---------------------------------------------------------------------------------------- |
| | • N Y I D K Q L L P I V N K Q S C S I S N I E T V I E |
| 1001 | AAAACTATAT AGATAAACAA TTGTTACCTA TTGTGAACAA GCAAAGCTGC AGCATATCAA ATATAGAAAC TGTGATAGAG |
| | TTTTGATATA TCTATTTGTT AACAATGGAT AACACTTGTT CGTTTCGACG TCGTATAGTT TATATCTTTG ACACTATCTC |
| | F2 |
| | --------------------- |
| | F Q Q K N N R • |
| | TTCCAACAAA AGAACAACAG |
| | AAGGTTGTTT TCTTGTTGTC |
| | F2 |
| | ---------------------------------------------------------------------------------------- |
| | • L L E I T R E F S V N A G V T T P V S T Y M L T N S E |
| 1101 | ACTACTAGAG ATTACCAGGG AATTTAGTGT TAATGCAGGT GTAACTACAC CTGTAAGCAC TTACATGTTA ACTAATAGTG |
| | TGATGATCTC TAATGGTCCC TTAAATCACA ATTACGTCCA CATTGATGTG GACATTCGTG AATGTACAAT TGATTATCAC |
| | F2 |
| | --------------------- |
| | L L S L I N |
| | AATTATTGTC ATTAATCAAT |
| | TTAATAACAG TAATTAGTTA |
| | F2 |
| | ---------------------------------------------------------------------------------------- |
| | D M P I T N D Q K K L M S N N V Q I V R Q Q S Y S I M |
| 1201 | GATATGCCTA TAACAAATGA TCAGAAAAAG TTAATGTCCA ACAATGTTCA AATAGTTAGA CAGCAAAGTT ACTCTATCAT |
| | CTATACGGAT ATTGTTTACT AGTCTTTTTC AATTACAGGT TGTTACAAGT TTATCAATCT GTCGTTTCAA TGAGATAGTA |
| | F2 |
| | --------------------- |
| | S I I K E E V • |
| | GTCCATAATA AAAGAGGAAG |
| | CAGGTATTAT TTTCTCCTTC |
| | F2 |
| | ---------------------------------------------------------------------------------------- |
| | • L A Y V V Q L P L Y G V I D T P C W K L H T S P L C |
| 1301 | TCTTAGCATA TGTAGTACAA TTACCACTAT ATGGTGTTAT AGATACACCC TGTTGGAAAC TACACACATC CCCTCTATGT |
| | AGAATCGTAT ACATCATGTT AATGGTGATA TACCACAATA TCTATGTGGG ACAACCTTTG ATGTGTGTAG GGGAGATACA |
| | F2 |
| | --------------------- |
| | T T N T K E G • |
| | ACAACCAACA CAAAAGAAGG |
| | TGTTGGTTGT GTTTTCTTCC |
| | F2 |
| | ---------------------------------------------------------------------------------------- |
| | • S N I C L T R T D R G W Y C D N A G S V S F F P Q A E |
| 1401 | GTCCAACATC TGTTTAACAA GAACTGACAG AGGATGGTAC TGTGACAATG CAGGATCAGT ATCTTTCTTC CCACAAGCTG |
| | CAGGTTGTAG ACAAATTGTT CTTGACTGTC TCCTACCATG ACACTGTTAC GTCCTAGTCA TAGAAAGAAG GGTGTTCGAC |
| | F2 |
| | --------------------- |
| | T C K V Q S |
| | AAACATGTAA AGTTCAATCA |
| | TTTGTACATT TCAAGTTAGT |
| | F2 |
| | ---------------------------------------------------------------------------------------- |
| | N R V F C D T M N S L T L P S E I N L C N V D I F N P |
| 1501 | AATCGAGTAT TTTGTGACAC AATGAACAGT TTAACATTAC CAAGTGAAAT AAATCTCTGC AATGTTGACA TATTCAACCC |
| | TTAGCTCATA AAACACTGTG TTACTTGTCA AATTGTAATG GTTCACTTTA TTTAGAGACG TTACAACTGT ATAAGTTGGG |
| | F2 |
| | --------------------- |
| | K Y D C K I M • |
| | CAAATATGAT TGTAAAATTA |
| | GTTTATACTA ACATTTTAAT |
| | F2 |
| | ---------------------------------------------------------------------------------------- |
| | • T S K T D V S S S V I T S L G A I V S C Y G K T K C |
| 1601 | TGACTTCAAA AACAGATGTA AGCAGCTCCG TTATCACATC TCTAGGAGCC ATTGTGTCAT GCTATGGCAA AACTAAATGT |
| | ACTGAAGTTT TTGTCTACAT TCGTCGAGGC AATAGTGTAG AGATCCTCGG TAACACAGTA CGATACCGTT TTGATTTACA |
| | F2 |
| | --------------------- |
| | T A S N K N R • |
| | ACAGCATCCA ATAAAAATCG |
| | TGTCGTAGGT TATTTTTAGC |
| | F2 |
| | ---------------------------------------------------------------------------------------- |
| | • G I I K T F S N G C D Y V S N K G M D T V S V G N T L |
| 1701 | TGGAATCATA AAGACATTTT CTAACGGGTG CGATTATGTA TCAAATAAAG GGATGGACAC TGTGTCTGTA GGTAACACAT |
| | ACCTTAGTAT TTCTGTAAAA GATTGCCCAC GCTAATACAT AGTTTATTTC CCTACCTGTG ACACAGACAT CCATTGTGTA |
| | F2 |
| | --------------------- |
| | Y Y V N K Q |
| | TATATTATGT AAATAAGCAA |
| | ATATAATACA TTTATTCGTT |
| | F2 |
| | ---------------------------------------------------------------------------------------- |
| | E G K S L Y V K G E P I I N F Y D P L V F P S D E F D |
| 1801 | GAAGGTAAAA GTCTCTATGT AAAAGGTGAA CCAATAATAA ATTTCTATGA CCCATTAGTA TTCCCCTCTG ATGAATTTGA |
| | CTTCCATTTT CAGAGATACA TTTTCCACTT GGTTATTATT TAAAGATACT GGGTAATCAT AAGGGGAGAC TACTTAAACT |
| | F2 |
| | --------------------- |
| | A S I S Q V N • |
| | TGCATCAATA TCTCAAGTCA |
| | ACGTAGTTAT AGAGTTCAGT |
| | F2 |
| | ---------------------------------------------------------------------------------------- |
| | • E K I N Q S L A F I R K S D E L L H N V N A G K S T |
| 1901 | ACGAGAAGAT TAACCAGAGC CTAGCATTTA TTCGTAAATC CGATGAATTA TTACATAATG TAAATGCTGG TAAATCCACC |
| | TGCTCTTCTA ATTGGTCTCG GATCGTAAAT AAGCATTTAG GCTACTTAAT AATGTATTAC ATTTACGACC ATTTAGGTGG |
| | F2 |
| | ------ |
| | TM Domain |
| | --------------- |
| | T N I M I T T • |
| | ACAAATATCA TGATAACTAC |
| | TGTTTATAGT ACTATTGATG |
| | TM Domain |
| | ------------------------------------------------------------ |
| | Cytoplasmic Tail |
| | ---------------------------- |
| | • I I I V I I V I L L S L I A V G L L L Y C K A R S T P |
| 2001 | TATAATTATA GTGATTATAG TAATATTGTT ATCATTAATT GCTGTTGGAC TGCTCTTATA CTGTAAGGCC AGAAGCACAC |
| | ATATTAATAT CACTAATATC ATTATAACAA TAGTAATTAA CGACAACCTG ACGAGAATAT GACATTCCGG TCTTCGTGTG |
| | Cytoplasmic Tail |
| | --------------------- |
| | V T L S K D |
| | CAGTCACACT AAGCAAAGAT |
| | GTCAGTGTGA TTCGTTTCTA |
| | FMDV 2A |
| | ------------------------------------------------- |
| | Cytoplasmic Tail |
| | --------------------------------------- |
| | Q L S G I N N I A F S N N F D L L K L A G D V E S N P |
| 2101 | CAACTGAGTG GTATAAATAA TATTGCATTT AGTAACAATT TTGATCTGCT CAAACTTGCA GGCGATGTAG AATCAAATCC |
| | GTTGACTCAC CATATTTATT ATAACGTAAA TCATTGTTAA AACTAGACGA GTTTGAACGT CCGCTACATC TTAGTTTAGG |
| | FMDV 2A |
| | ------- |
| | Transmembrane domain of WNV E (split) |
| | ---- |
| | pre E/NS1 signal |
| | ---------- |
| | G P A R D R S • |
| | TGGACCCGCC CGGGACAGGT |
| | ACCTGGGCGG GCCCTGTCCA |
| | NS1 |
| | ----------------- |
| | Transmembrane domain of WNV E (split) |
| | ----------------------------------------------------------------------- |
| | • I A L T F L A V G G V L L F L S V N V H A D T G C A |
| 2201 | CCATAGCTCT CACGTTTCTC GCAGTTGGAG GAGTTCTGCT CTTCCTCTCC GTGAACGTGC ACGCTGACAC TGGGTGTGCC |
| | GGTATCGAGA GTGCAAAGAG CGTCAACCTC CTCAAGACGA GAAGGAGAGG CACTTGCACG TGCGACTGTG ACCCACACGG |
| | NS1 |
| | ------------------- |
| | I D I S R Q |
| | ATAGACATCA GCCGGCAA |
| | TATCTGTAGT CGGCCGTT |

PIV-WNV(ΔCprME)/RSV-F

| | |
|---|---|
| | 5′ UTR |
| | ---------------------------------------------------------------------------------------- |
| 1 | AGTAGTTCGC CTGTGTGAGC TGACAAACTT AGTAGTGTTT GTGAGGATTA ACAACAATTA ACACAGTGCG AGCTGTTTCT |
| | TCATCAAGCG GACACACTCG ACTGTTTGAA TCATCACAAA CACTCCTAAT TGTTGTTAAT TGTGTCACGC TCGACAAAGA |
| | deleted C protein |
| | ---- |
| | 5′ UTR |
| | ----------------- |
| | M S • |
| | TAGCACGAAG ATCTCGATGT |
| | ATCGTGCTTC TAGAGCTACA |
| | deleted C protein |
| | ---------------------------------------------------------------------------------------- |
| | • K K P G G P G K S R A V N M L K R G M P R V L S L I |
| 101 | CTAAGAAACC AGGAGGGCCC GGCAAGAGCC GGGCTGTCAA TATGCTAAAA CGCGGAATGC CCCGCGTGTT GTCCTTGATT |
| | GATTCTTTGG TCCTCCCGGG CCGTTCTCGG CCCGACAGTT ATACGATTTT GCGCCTTACG GGGCGCACAA CAGGAACTAA |
| | deleted C protein |
| | ------ |
| | NS3 cleavage |
| | --------------- |
| | G L K Q K K R • |
| | GGACTTAAGC AAAAGAAGCG |
| | CCTGAATTCG TTTTCTTCGC |
| | F signal |
| | ---------------------------------------------------------------------------- |
| | NS3 cleavage F1 |
| | - ----------- |
| | • G G E L L I L K A N A I T T I L T A V T F C F A S G Q |
| 201 | AGGGGGCGAG TTGCTAATCC TCAAAGCAAA TGCAATTACC ACAATCCTCA CTGCAGTCAC ATTTTGTTTT GCTTCTGGTC |
| | TCCCCCGCTC AACGATTAGG AGTTTCGTTT ACGTTAATGG TGTTAGGAGT GACGTCAGTG TAAAACAAAA CGAAGACCAG |
| | F1 |
| | ---------------------- |
| | N I T E E F |
| | AAAACATCAC TGAAGAATTT |
| | TTTTGTAGTG ACTTCTTAAA |
| | F1 |
| | ---------------------------------------------------------------------------------------- |
| | Y Q S T C S A V S K G Y L S A L R T G W Y T S V I T I |
| 301 | TATCAATCAA CATGCAGTGC AGTTAGCAAA GGCTATCTTA GTGCTCTGAG AACTGGTTGG TATACCAGTG TTATAACTAT |
| | ATAGTTAGTT GTACGTCACG TCAATCGTTT CCGATAGAAT CACGAGACTC TTGACCAACC ATATGGTCAC AATATTGATA |
| | F1 |
| | --------------------- |
| | E L S N I K E • |
| | AGAATTAAGT AATATCAAGG |
| | TCTTAATTCA TTATAGTTCC |
| | F1 |
| | ---------------------------------------------------------------------------------------- |
| | • N K C N G T D A K V K L I K Q E L D K Y K N A V T E |
| 401 | AAAATAAGTG TAATGGAACA GATGCTAAGG TAAAATTGAT AAAACAAGAA TTAGATAAAT ATAAAAATGC TGTAACAGAA |
| | TTTTATTCAC ATTACCTTGT CTACGATTCC ATTTTAACTA TTTTGTTCTT AATCTATTTA TATTTTTACG ACATTGTCTT |
| | F1 |
| | --------------------- |
| | L Q L L M Q S • |
| | TTGCAGTTGC TCATGCAAAG |
| | AACGTCAACG AGTACGTTTC |
| | F1 |
| | ---------------------------------------------------------------------------------------- |
| | • T P P T N N R A R R E L P R F M N Y T L N N A K K T N |
| 501 | CACACCACCA ACAAACAATC GAGCCAGAAG AGAACTACCA AGGTTTATGA ATTATACACT CAACAATGCC AAAAAAACCA |
| | GTGTGGTGGT TGTTTGTTAG CTCGGTCTTC TCTTGATGGT TCCAAATACT TAATATGTGA GTTGTTACGG TTTTTTTGGT |
| | F1 |
| | --------------------- |
| | V T L S K K |
| | ATGTAACATT AAGCAAGAAA |
| | TACATTGTAA TTCGTTCTTT |
| | F1 |
| | ------------- |
| | F2 |
| | --------------------------------------------------------------------------- |
| | R K R R F L G F L L G V G S A I A S G V A V S K V L H |
| 601 | AGGAAAAGAA GATTTCTTGG TTTTTTGTTA GGTGTTGGAT CTGCAATCGC CAGTGGCGTT GCTGTATCTA AGGTCCTGCA |
| | TCCTTTTCTT CTAAAGAACC AAAAAACAAT CCACAACCTA GACGTTAGCG GTCACCGCAA CGACATAGAT TCCAGGACGT |
| | F2 |
| | --------------------- |
| | L E G E V N K • |
| | CCTAGAAGGG GAAGTGAACA |
| | GGATCTTCCC CTTCACTTGT |
| | F2 |
| | ---------------------------------------------------------------------------------------- |
| | • I K S A L L S T N K A V V S L S N G V S V L T S K V |
| 701 | AGATCAAAAG TGCTCTACTA TCCACAAACA AGGCTGTAGT CAGCTTATCA AATGGAGTTA GTGTCTTAAC CAGCAAAGTG |
| | TCTAGTTTTC ACGAGATGAT AGGTGTTTGT TCCGACATCA GTCGAATAGT TTACCTCAAT CACAGAATTG GTCGTTTCAC |
| | F2 |
| | --------------------- |
| | L D L K N Y I • |
| | TTAGACCTCA AAAACTATAT |
| | AATCTGGAGT TTTTGATATA |
| | F2 |
| | ---------------------------------------------------------------------------------------- |
| | • D K Q L L P I V N K Q S C S I S N I E T V I E F Q Q K |
| 801 | AGATAAACAA TTGTTACCTA TTGTGAACAA GCAAAGCTGC AGCATATCAA ATATAGAAAC TGTGATAGAG TTCCAACAAA |
| | TCTATTTGTT AACAATGGAT AACACTTGTT CGTTTCGACG TCGTATAGTT TATATCTTTG ACACTATCTC AAGGTTGTTT |
| | F2 |
| | --------------------- |
| | N N R L L E |
| | AGAACAACAG ACTACTAGAG |
| | TCTTGTTGTC TGATGATCTC |
| | F2 |
| | ---------------------------------------------------------------------------------------- |
| | I T R E F S V N A G V T T P V S T Y M L T N S E L L S |
| 901 | ATTACCAGGG AATTTAGTGT TAATGCAGGT GTAACTACAC CTGTAAGCAC TTACATGTTA ACTAATAGTG AATTATTGTC |
| | TAATGGTCCC TTAAATCACA ATTACGTCCA CATTGATGTG GACATTCGTG AATGTACAAT TGATTATCAC TTAATAACAG |
| | F2 |
| | --------------------- |
| | L I N D M P I • |
| | ATTAATCAAT GATATGCCTA |
| | TAATTAGTTA CTATACGGAT |
| | F2 |
| | ---------------------------------------------------------------------------------------- |
| | • T N D Q K K L M S N N V Q I V R Q Q S Y S I M S I I |
| 1001 | TAACAAATGA TCAGAAAAAG TTAATGTCCA ACAATGTTCA AATAGTTAGA CAGCAAAGTT ACTCTATCAT GTCCATAATA |
| | ATTGTTTACT AGTCTTTTTC AATTACAGGT TGTTACAAGT TTATCAATCT GTCGTTTCAA TGAGATAGTA CAGGTATTAT |
| | F2 |
| | --------------------- |
| | K E E V L A Y • |
| | AAAGAGGAAG TCTTAGCATA |
| | TTTCTCCTTC AGAATCGTAT |
| | F2 |
| | ---------------------------------------------------------------------------------------- |
| | • V V Q L P L Y G V I D T P C W K L H T S P L C T T N T |
| 1101 | TGTAGTACAA TTACCACTAT ATGGTGTTAT AGATACACCC TGTTGGAAAC TACACACATC CCCTCTATGT ACAACCAACA |
| | ACATCATGTT AATGGTGATA TACCACAATA TCTATGTGGG ACAACCTTTG ATGTGTGTAG GGGAGATACA TGTTGGTTGT |
| | F2 |
| | --------------------- |
| | K E G S N I |
| | CAAAAGAAGG GTCCAACATC |
| | GTTTTCTTCC CAGGTTGTAG |
| | F2 |
| | ---------------------------------------------------------------------------------------- |
| | C L T R T D R G W Y C D N A G S V S F F P Q A E T C K |
| 1201 | TGTTTAACAA GAACTGACAG AGGATGGTAC TGTGACAATG CAGGATCAGT ATCTTTCTTC CCACAAGCTG AAACATGTAA |
| | ACAAATTGTT CTTGACTGTC TCCTACCATG ACACTGTTAC GTCCTAGTCA TAGAAAGAAG GGTGTTCGAC TTTGTACATT |
| | F2 |
| | --------------------- |
| | V Q S N R V F • |
| | AGTTCAATCA AATCGAGTAT |
| | TCAAGTTAGT TTAGCTCATA |
| | F2 |
| | ---------------------------------------------------------------------------------------- |
| | • C D T M N S L T L P S E I N L C N V D I F N P K Y D |
| 1301 | TTTGTGACAC AATGAACAGT TTAACATTAC CAAGTGAAAT AAATCTCTGC AATGTTGACA TATTCAACCC CAAATATGAT |
| | AAACACTGTG TTACTTGTCA AATTGTAATG GTTCACTTTA TTTAGAGACG TTACAACTGT ATAAGTTGGG GTTTATACTA |
| | F2 |
| | --------------------- |
| | C K I M T S K • |
| | TGTAAAATTA TGACTTCAAA |
| | ACATTTTAAT ACTGAAGTTT |
| | F2 |
| | ---------------------------------------------------------------------------------------- |
| | • T D V S S S V I T S L G A I V S C Y G K T K C T A S N |
| 1401 | AACAGATGTA AGCAGCTCCG TTATCACATC TCTAGGAGCC ATTGTGTCAT GCTATGGCAA AACTAAATGT ACAGCATCCA |
| | TTGTCTACAT TCGTCGAGGC AATAGTGTAG AGATCCTCGG TAACACAGTA CGATACCGTT TTGATTTACA TGTCGTAGGT |
| | F2 |
| | --------------------- |
| | K N R G I I |
| | ATAAAAATCG TGGAATCATA |
| | TATTTTTAGC ACCTTAGTAT |
| | F2 |
| | ---------------------------------------------------------------------------------------- |
| | K T F S N G C D Y V S N K G M D T V S V G N T L Y Y V |
| 1501 | AAGACATTTT CTAACGGGTG CGATTATGTA TCAAATAAAG GGATGGACAC TGTGTCTGTA GGTAACACAT TATATTATGT |
| | TTCTGTAAAA GATTGCCCAC GCTAATACAT AGTTTATTTC CCTACCTGTG ACACAGACAT CCATTGTGTA ATATAATACA |
| | F2 |
| | --------------------- |
| | N K Q E G K S • |
| | AAATAAGCAA GAAGGTAAAA |
| | TTTATTCGTT CTTCCATTTT |
| | F2 |
| | ---------------------------------------------------------------------------------------- |
| | • L Y V K G E P I I N F Y D P L V F P S D E F D A S I |
| 1601 | GTCTCTATGT AAAAGGTGAA CCAATAATAA ATTTCTATGA CCCATTAGTA TTCCCCTCTG ATGAATTTGA TGCATCAATA |
| | CAGAGATACA TTTTCCACTT GGTTATTATT TAAAGATACT GGGTAATCAT AAGGGGAGAC TACTTAAACT ACGTAGTTAT |
| | F2 |
| | --------------------- |
| | S Q V N E K I • |
| | TCTCAAGTCA ACGAGAAGAT |
| | AGAGTTCAGT TGCTCTTCTA |
| | F2 |
| | ------------------------------------------------------------------------------------ |
| | TM |
| | Domain |
| | ----- |
| | • N Q S L A F I R K S D E L L H N V N A G K S T T N I M |
| 1701 | TAACCAGAGC CTAGCATTTA TTCGTAAATC CGATGAATTA TTACATAATG TAAATGCTGG TAAATCCACC ACAAATATCA |
| | ATTGGTCTCG GATCGTAAAT AAGCATTTAG GCTACTTAAT AATGTATTAC ATTTACGACC ATTTAGGTGG TGTTTATAGT |
| | TM Domain |
| | --------------------- |
| | I T T I I I |
| | TGATAACTAC TATAATTATA |
| | ACTATTGATG ATATTAATAT |
| | TM Domain |
| | ------------------------------------------------- |
| | Cytoplasmic Tail |
| | --------------------------------------- |
| | V I I V I L L S L I A V G L L L Y C K A R S T P V T L |
| 1801 | GTGATTATAG TAATATTGTT ATCATTAATT GCTGTTGGAC TGCTCTTATA CTGTAAGGCC AGAAGCACAC CAGTCACACT |
| | CACTAATATC ATTATAACAA TAGTAATTAA CGACAACCTG ACGAGAATAT GACATTCCGG TCTTCGTGTG GTCAGTGTGA |
| | Cytoplasmic Tail |
| | --------------------- |
| | S K D Q L S G • |
| | AAGCAAAGAT CAACTGAGTG |
| | TTCGTTTCTA GTTGACTCAC |
| | FMDV 2A |
| | -------------------------------------------------------- |
| | pre E/NS1 |
| | Cytoplasmic Tail signal |
| | ---------------------------- ---- |
| | • I N N I A F S N N F D L L K L A G D V E S N P G P A |
| 1901 | GTATAAATAA TATTGCATTT AGTAACAATT TTGATCTGCT CAAACTTGCA GGCGATGTAG AATCAAATCC TGGACCCGCC |
| | CATATTTATT ATAACGTAAA TCATTGTTAA AACTAGACGA GTTTGAACGT CCGCTACATC TTAGTTTAGG ACCTGGGCGG |
| | membrane domain of WNV E (split) |
| | --------------- |
| | pre E/NS1 signal |
| | ------ |
| | R D R S I A L • |
| | CGGGACAGGT CCATAGCTCT |
| | GCCCTGTCCA GGTATCGAGA |
| | Transmembrane domain of WNV E (split) |
| | ------------------------------------------------------------ |
| | NS1 |
| | ---------------------------- |
| | • T F L A V G G V L L F L S V N V H A D T G C A I D I S |
| 2001 | CACGTTTCTC GCAGTTGGAG GAGTTCTGCT CTTCCTCTCC GTGAACGTGC ACGCTGACAC TGGGTGTGCC ATAGACATCA |
| | GTGCAAAGAG CGTCAACCTC CTCAAGACGA GAAGGAGAGG CACTTGCACG TGCGACTGTG ACCCACACGG TATCTGTAGT |
| | NS1 |
| | ----------------- |
| | R Q E L R |
| | GCCGGCAAGA GCTGAGA |
| | CGGCCGTTCT CGACTCT |

PIV-WNV(ΔC)/RSV-F

| | |
|---|---|
| 1 | GATCCTAATA CGACTCACTA TAGAGTAGTT CGCCTGTGTG AGCTGACAAA CTTAGTAGTG TTTGTGAGGA TTAACAACAA |
| | CTAGGATTAT GCTGAGTGAT ATCTCATCAA GCGGACACAC TCGACTGTTT GAATCATCAC AAACACTCCT AATTGTTGTT |
| | TTAACACAGT GCGAGCTGTT |
| | AATTGTGTCA CGCTCGACAA |
| | N-terminus of C |
| | -------------------------------------------------------------------- |
| | M S K K P G G P G K S R A V N M L K R G M |
| 101 | TCTTAGCACG AAGATCTCGA TGTCTAAGAA ACCAGGAGGG CCCGGCAAGA GCCGGGCTGT CAATATGCTA AAACGCGGAA |
| | AGAATCGTGC TTCTAGAGCT ACAGATTCTT TGGTCCTCCC GGGCCGTTCT CGGCCCGACA GTTATACGAT TTTGCGCCTT |
| | N-terminus of C |
| | --------------------- |
| | P R V L S L |
| | TGCCCCGCGT GTTGTCCTTG |
| | ACGGGGCGCA CAACAGGAAC |
| | N-terminus of C F signal |
| | --------- -------------------------------------------------------------- |
| | NS3 cleavage |
| | ----------------- |
| | I G L K Q K K R G G E L L I L K A N A I T T I L T A V |
| 201 | ATTGGACTTA AGCAAAAGAA GCGAGGGGGC GAGTTGCTAA TCCTCAAAGC AAATGCAATT ACCACAATCC TCACTGCAGT |
| | TAACCTGAAT TCGTTTTCTT CGCTCCCCCG CTCAACGATT AGGAGTTTCG TTTACGTTAA TGGTGTTAGG AGTGACGTCA |
| | F signal |
| | -------------- |
| | F1 |
| | ------- |
| | T F C F A S G • |
| | CACATTTTGT TTTGCTTCTG |
| | GTGTAAAACA AAACGAAGAC |
| | F1 |
| | ---------------------------------------------------------------------------------------- |
| | • Q N I T E E F Y Q S T C S A V S K G Y L S A L R T G |
| 301 | GTCAAAACAT CACTGAAGAA TTTTATCAAT CAACATGCAG TGCAGTTAGC AAAGGCTATC TTAGTGCTCT GAGAACTGGT |
| | CAGTTTTGTA GTGACTTCTT AAAATAGTTA GTTGTACGTC ACGTCAATCG TTTCCGATAG AATCACGAGA CTCTTGACCA |
| | F1 |
| | --------------------- |
| | W Y T S V I T • |
| | TGGTATACCA GTGTTATAAC |
| | ACCATATGGT CACAATATTG |
| | F1 |
| | ---------------------------------------------------------------------------------------- |
| | • I E L S N I K E N K C N G T D A K V K L I K Q E L D K |
| 401 | TATAGAATTA AGTAATATCA AGGAAAATAA GTGTAATGGA ACAGATGCTA AGGTAAAATT GATAAAACAA GAATTAGATA |
| | ATATCTTAAT TCATTATAGT TCCTTTTATT CACATTACCT TGTCTACGAT TCCATTTTAA CTATTTTGTT CTTAATCTAT |
| | F1 |
| | --------------------- |
| | Y K N A V T |
| | AATATAAAAA TGCTGTAACA |
| | TTATATTTTT ACGACATTGT |
| | F1 |
| | ---------------------------------------------------------------------------------------- |
| | E L Q L L M Q S T P P T N N R A R R E L P R F M N Y T |
| 501 | GAATTGCAGT TGCTCATGCA AAGCACACCA CCAACAAACA ATCGAGCCAG AAGAGAACTA CCAAGGTTTA TGAATTATAC |
| | CTTAACGTCA ACGAGTACGT TTCGTGTGGT GGTTGTTTGT TAGCTCGGTC TTCTCTTGAT GGTTCCAAAT ACTTAATATG |
| | F1 |
| | --------------------- |
| | L N N A K K T • |
| | ACTCAACAAT GCCAAAAAAA |
| | TGAGTTGTTA CGGTTTTTTT |
| | F2 |
| | -------------------------------------------------- |
| | F1 |
| | -------------------------------------- |
| | • N V T L S K K R K R R F L G F L L G V G S A I A S G |
| 601 | CCAATGTAAC ATTAAGCAAG AAAAGGAAAA GAAGATTTCT TGGTTTTTTG TTAGGTGTTG GATCTGCAAT CGCCAGTGGC |
| | GGTTACATTG TAATTCGTTC TTTTCCTTTT CTTCTAAAGA ACCAAAAAAC AATCCACAAC CTAGACGTTA GCGGTCACCG |
| | F2 |
| | --------------------- |
| | V A V S K V L • |
| | GTTGCTGTAT CTAAGGTCCT |
| | CAACGACATA GATTCCAGGA |
| | F2 |
| | ---------------------------------------------------------------------------------------- |
| | • H L E G E V N K I K S A L L S T N K A V V S L S N G V |
| 701 | GCACCTAGAA GGGGAAGTGA ACAAGATCAA AAGTGCTCTA CTATCCACAA ACAAGGCTGT AGTCAGCTTA TCAAATGGAG |
| | CGTGGATCTT CCCCTTCACT TGTTCTAGTT TTCACGAGAT GATAGGTGTT TGTTCCGACA TCAGTCGAAT AGTTTACCTC |
| | F2 |
| | --------------------- |
| | S V L T S K |
| | TTAGTGTCTT AACCAGCAAA |
| | AATCACAGAA TTGGTCGTTT |
| | F2 |
| | ---------------------------------------------------------------------------------------- |
| | V L D L K N Y I D K Q L L P I V N K Q S C S I S N I E |
| 801 | GTGTTAGACC TCAAAAACTA TATAGATAAA CAATTGTTAC CTATTGTGAA CAAGCAAAGC TGCAGCATAT CAAATATAGA |
| | CACAATCTGG AGTTTTTGAT ATATCTATTT GTTAACAATG GATAACACTT GTTCGTTTCG ACGTCGTATA GTTTATATCT |
| | F2 |
| | --------------------- |
| | T V I E F Q Q • |
| | AACTGTGATA GAGTTCCAAC |
| | TTGACACTAT CTCAAGGTTG |
| | F2 |
| | ---------------------------------------------------------------------------------------- |
| | • K N N R L L E I T R E F S V N A G V T T P V S T Y M |
| 901 | AAAAGAACAA CAGACTACTA GAGATTACCA GGGAATTTAG TGTTAATGCA GGTGTAACTA CACCTGTAAG CACTTACATG |
| | TTTTCTTGTT GTCTGATGAT CTCTAATGGT CCCTTAAATC ACAATTACGT CCACATTGAT GTGGACATTC GTGAATGTAC |
| | F2 |
| | --------------------- |
| | L T N S E L L • |
| | TTAACTAATA GTGAATTATT |
| | AATTGATTAT CACTTAATAA |
| | F2 |
| | ---------------------------------------------------------------------------------------- |
| | • S L I N D M P I T N D Q K K L M S N N V Q I V R Q Q S |
| 1001 | GTCATTAATC AATGATATGC CTATAACAAA TGATCAGAAA AAGTTAATGT CCAACAATGT TCAAATAGTT AGACAGCAAA |
| | CAGTAATTAG TTACTATACG GATATTGTTT ACTAGTCTTT TTCAATTACA GGTTGTTACA AGTTTATCAA TCTGTCGTTT |
| | F2 |
| | --------------------- |
| | Y S I M S I |
| | GTTACTCTAT CATGTCCATA |
| | CAATGAGATA GTACAGGTAT |
| | F2 |
| | ---------------------------------------------------------------------------------------- |
| | I K E E V L A Y V V Q L P L Y G V I D T P C W K L H T |
| 1101 | ATAAAAGAGG AAGTCTTAGC ATATGTAGTA CAATTACCAC TATATGGTGT TATAGATACA CCCTGTTGGA AACTACACAC |
| | TATTTTCTCC TTCAGAATCG TATACATCAT GTTAATGGTG ATATACCACA ATATCTATGT GGGACAACCT TTGATGTGTG |
| | F2 |
| | --------------------- |
| | S P L C T T N • |
| | ATCCCCTCTA TGTACAACCA |
| | TAGGGGAGAT ACATGTTGGT |
| | F2 |
| | ---------------------------------------------------------------------------------------- |
| | • T K E G S N I C L T R T D R G W Y C D N A G S V S F |
| 1201 | ACACAAAAGA AGGGTCCAAC ATCTGTTTAA CAAGAACTGA CAGAGGATGG TACTGTGACA ATGCAGGATC AGTATCTTTC |
| | TGTGTTTTCT TCCCAGGTTG TAGACAAATT GTTCTTGACT GTCTCCTACC ATGACACTGT TACGTCCTAG TCATAGAAAG |
| | F2 |
| | --------------------- |
| | F P Q A E T C • |
| | TTCCCACAAG CTGAAACATG |
| | AAGGGTGTTC GACTTTGTAC |
| | F2 |
| | ---------------------------------------------------------------------------------------- |
| | • K V Q S N R V F C D T M N S L T L P S E I N L C N V D |
| 1301 | TAAAGTTCAA TCAAATCGAG TATTTTGTGA CACAATGAAC AGTTTAACAT TACCAAGTGA AATAAATCTC TGCAATGTTG |
| | ATTTCAAGTT AGTTTAGCTC ATAAAACACT GTGTTACTTG TCAAATTGTA ATGGTTCACT TTATTTAGAG ACGTTACAAC |
| | F2 |
| | --------------------- |
| | I F N P K Y |
| | ACATATTCAA CCCCAAATAT |
| | TGTATAAGTT GGGGTTTATA |
| | F2 |
| | ---------------------------------------------------------------------------------------- |
| | D C K I M T S K T D V S S S V I T S L G A I V S C Y G |
| 1401 | GATTGTAAAA TTATGACTTC AAAAACAGAT GTAAGCAGCT CCGTTATCAC ATCTCTAGGA GCCATTGTGT CATGCTATGG |
| | CTAACATTTT AATACTGAAG TTTTTGTCTA CATTCGTCGA GGCAATAGTG TAGAGATCCT CGGTAACACA GTACGATACC |
| | F2 |
| | --------------------- |
| | K T K C T A S • |
| | CAAAACTAAA TGTACAGCAT |
| | GTTTTGATTT ACATGTCGTA |
| | F2 |
| | ---------------------------------------------------------------------------------------- |
| | • N K N R G I I K T F S N G C D Y V S N K G M D T V S |
| 1501 | CCAATAAAAA TCGTGGAATC ATAAAGACAT TTTCTAACGG GTGCGATTAT GTATCAAATA AAGGGATGGA CACTGTGTCT |
| | GGTTATTTTT AGCACCTTAG TATTTCTGTA AAAGATTGCC CACGCTAATA CATAGTTTAT TTCCCTACCT GTGACACAGA |
| | F2 |
| | --------------------- |
| | V G N T L Y Y • |
| | GTAGGTAACA CATTATATTA |
| | CATCCATTGT GTAATATAAT |
| | F2 |
| | ---------------------------------------------------------------------------------------- |
| | • V N K Q E G K S L Y V K G E P I I N F Y D P L V F P S |
| 1601 | TGTAAATAAG CAAGAAGGTA AAAGTCTCTA TGTAAAAGGT GAACCAATAA TAAATTTCTA TGACCCATTA GTATTCCCCT |
| | ACATTTATTC GTTCTTCCAT TTTCAGAGAT ACATTTTCCA CTTGGTTATT ATTTAAAGAT ACTGGGTAAT CATAAGGGGA |
| | F2 |
| | --------------------- |
| | D E F D A S |
| | CTGATGAATT TGATGCATCA |
| | GACTACTTAA ACTACGTAGT |
| | F2 |
| | ---------------------------------------------------------------------------------------- |
| | I S Q V N S K I N Q S L A F I R K S D E L L H N V N A |
| 1701 | ATATCTCAAG TCAACGAGAA GATTAACCAG AGCCTAGCAT TTATTCGTAA ATCCGATGAA TTATTACATA ATGTAAATGC |
| | TATAGAGTTC AGTTGCTCTT CTAATTGGTC TCGGATCGTA AATAAGCATT TAGGCTACTT AATAATGTAT TACATTTACG |
| | F2 |
| | -------------------- |
| | TM Domain |
| | - |
| | G K S T T N I • |
| | TGGTAAATCC ACCACAAATA |
| | ACCATTTAGG TGGTGTTTAT |
| | TM Domain |
| | -------------------------------------------------------------------------- |
| | Cytoplasmic |
| | Tail |
| | -------------- |
| | • M I T T I I I V I I V I L L S L I A V G L L L Y C K |
| 1801 | TCATGATAAC TACTATAATT ATAGTGATTA TAGTAATATT GTTATCATTA ATTGCTGTTG GACTGCTCTT ATACTGTAAG |
| | AGTACTATTG ATGATATTAA TATCACTAAT ATCATTATAA CAATAGTAAT TAACGACAAC CTGACGAGAA TATGACATTC |
| | Cytoplasmic Tail |
| | --------------------- |
| | A R S T P V T • |
| | GCCAGAAGCA CACCAGTCAC |
| | CGGTCTTCGT GTGGTCAGTG |
| | FMDV 2A |
| | ----------------------------------- |
| | Cytoplasmic Tail |
| | ----------------------------------------------------- |
| | • L S K D Q L S G I N N I A F S N N F D L L K L A G D V |
| 1901 | ACTAAGCAAA GATCAACTGA GTGGTATAAA TAATATTGCA TTTAGTAACA ATTTTGATCT GCTCAAACTT GCAGGCGATG |
| | TGATTCGTTT CTAGTTGACT CACCATATTT ATTATAACGT AAATCATTGT TAAAACTAGA CGAGTTTGAA CGTCCGCTAC |
| | FMDV 2A |
| | --------------------- |
| | E S N P G P |
| | TAGAATCAAA TCCTGGACCC |
| | ATCTTAGTTT AGGACCTGGG |
| | prM |
| | ----------------------------- |
| | C/prM signal |
| | ----------------------------------------------------------- |
| | G G K T G I A V M I G L I A C V G A V T L S N F Q G K |
| 2001 | GGAGGAAAGA CCGGTATTGC AGTCATGATT GGCCTGATCG CCTGCGTAGG AGCAGTTACC CTCTCTAACT TCCAAGGGAA |
| | CCTCCTTTCT GGCCATAACG TCAGTACTAA CCGGACTAGC GGACGCATCC TCGTCAATGG GAGAGATTGA AGGTTCCCTT |
| | prM |
| | --------------------- |
| | V M M T V N A • |
| | GGTGATGATG ACGGTAAATG |
| | CCACTACTAC TGCCATTTAC |
| | prM |
| | ---------------------------------------------------------------------------------------- |
| | • T D V T D V I T I P T A A G K N L C I V R A M D V G |
| 2101 | CTACTGACGT CACAGATGTC ATCACGATTC CAACAGCTGC TGGAAAGAAC CTATGCATTG TCAGAGCAAT GGATGTGGGA |
| | GATGACTGCA GTGTCTACAG TAGTGCTAAG GTTGTCGACG ACCTTTCTTG GATACGTAAC AGTCTCGTTA CCTACACCCT |
| | prM |
| | --------------------- |
| | Y M C D D T I • |
| | TACATGTGCG ATGATACTAT |
| | ATGTACACGC TACTATGATA |
| | prM |
| | ---------------------------------------------------------------------------------------- |
| | • T Y E C P V L S A G N D P E D I D C W C T K S A V Y V |
| 2201 | CACTTATGAA TGCCCAGTGC TGTCGGCTGG TAATGATCCA GAAGACATCG ACTGTTGGTG CACAAAGTCA GCAGTCTACG |
| | GTGAATACTT ACGGGTCACG ACAGCCGACC ATTACTAGGT CTTCTGTAGC TGACAACCAC GTGTTTCAGT CGTCAGATGC |
| | prM |
| | --------------------- |
| | R Y G R C T |
| | TCAGGTATGG AAGATGCACC |
| | AGTCCATACC TTCTACGTGG |
| | prM |
| | ---------------------------------------------------------------------------------------- |
| | K T R H S R R S R R S L T V Q T H G E S T L A N K K G |
| 2301 | AAGACACGCC ACTCAAGACG CAGTCGGAGG TCACTGACAG TGCAGACACA CGGAGAAAGC ACTCTAGCGA ACAAGAAGGG |
| | TTCTGTGCGG TGAGTTCTGC GTCAGCCTCC AGTGACTGTC ACGTCTGTGT GCCTCTTTCG TGAGATCGCT TGTTCTTCCC |
| | prM |
| | --------------------- |
| | A W M D S T K • |
| | GGCTTGGATG GACAGCACCA |
| | CCGAACCTAC CTGTCGTGGT |
| | prM |
| | ---------------------------------------------------------------------------------------- |
| | • A T R Y L V K T E S W I L R N P G Y A L V A A V I G |
| 2401 | AGGCCACAAG GTATTTGGTA AAAACAGAAT CATGGATCTT GAGGAACCCT GGATATGCCC TGGTGGCAGC CGTCATTGGT |
| | TCCGGTGTTC CATAAACCAT TTTTGTCTTA GTACCTAGAA CTCCTTGGGA CCTATACGGG ACCACCGTCG GCAGTAACCA |
| | prM |
| | --------------------- |
| | W M L G S N T • |
| | TGGATGCTTG GGAGCAACAC |
| | ACCTACGAAC CCTCGTTGTG |
| | prM |
| | ---------------------------------------------------------------------------------------- |
| | • M Q R V V F V V L L L L V A P A Y S F N C L G M S N R |
| 2501 | CATGCAGAGA GTTGTGTTTG TCGTGCTATT GCTTTTGGTG GCCCCAGCTT ACAGCTTTAA CTGCCTTGGA ATGAGCAACA |
| | GTACGTCTCT CAACACAAAC AGCACGATAA CGAAAACCAC CGGGGTCGAA TGTCGAAATT GACGGAACCT TACTCGTTGT |
| | prM |
| | --------------------- |
| | D F L E G V |
| | GAGACTTCTT GGAAGGAGTG |
| | CTCTGAAGAA CCTTCCTCAC |
| | prM |
| | ------------------------------------------------------------------------------------- |
| | E |
| | --- |
| | S G A T W V D L V L E G D S C V T I M S K D K P T I D |
| 2601 | TCTGGAGCAA CATGGGTGGA TTTGGTTCTC GAAGGCGACA GCTGCGTGAC TATCATGTCT AAGGACAAGC CTACCATCGA |
| | AGACCTCGTT GTACCCACCT AAACCAAGAG CTTCCGCTGT CGACGCACTG ATAGTACAGA TTCCTGTTCG GATGGTAGCT |
| | E |
| | --------------------- |
| | V K M M N M E • |
| | TGTGAAGATG ATGAATATGG |
| | ACACTTCTAC TACTTATACC |
| | E |
| | ---------------------------------------------------------------------------------------- |
| | • A A N L A E V R S Y C Y L A T V S D L S T K A A C P |
| 2701 | AGGCGGCCAA CCTGGCAGAG GTCCGCAGTT ATTGCTATTT GGCTACCGTC AGCGATCTCT CCACCAAAGC TGCGTGCCCG |
| | TCCGCCGGTT GGACCGTCTC CAGGCGTCAA TAACGATAAA CCGATGGCAG TCGCTAGAGA GGTGGTTTCG ACGCACGGGC |
| | E |
| | --------------------- |
| | A M G E A H N • |
| | GCCATGGGAG AAGCTCACAA |
| | CGGTACCCTC TTCGAGTGTT |
| | E |
| | ---------------------------------------------------------------------------------------- |
| | • D K R A D P A F V C R Q G V V D R G W G N G C G L F G |
| 2801 | TGACAAACGT GCTGACCCAG CTTTTGTGTG CAGACAAGGA GTGGTGGACA GGGGCTGGGG CAACGGCTGC GGACTATTTG |
| | ACTGTTTGCA CGACTGGGTC GAAAACACAC GTCTGTTCCT CACCACCTGT CCCCGACCCC GTTGCCGACG CCTGATAAAC |
| | E |
| | --------------------- |
| | K G S I D T |
| | GCAAAGGAAG CATTGACACA |
| | CGTTTCCTTC GTAACTGTGT |
| | E |
| | ---------------------------------------------------------------------------------------- |
| | C A K F A C S T K A I G R T I L K E N I K Y E V A I F |
| 2901 | TGCGCCAAAT TTGCCTGCTC TACCAAGGCA ATAGGAAGAA CCATTTTGAA AGAGAATATC AAGTACGAAG TGGCCATTTT |
| | ACGCGGTTTA AACGGACGAG ATGGTTCCGT TATCCTTCTT GGTAAAACTT TCTCTTATAG TTCATGCTTC ACCGGTAAAA |
| | E |
| | --------------------- |
| | V H G P T T V • |
| | TGTCCATGGA CCAACTACTG |
| | ACAGGTACCT GGTTGATGAC |
| | E |
| | ---------------------------------------------------------------------------------------- |
| | • E S H G N Y S T Q V G A T Q A G R F S I T P A A P S |
| 3001 | TGGAGTCGCA CGGAAACTAC TCCACACAGG TTGGAGCCAC TCAGGCAGGG AGATTCAGCA TCACTCCTGC GGCGCCTTCA |
| | ACCTCAGCGT GCCTTTGATG AGGTGTGTCC AACCTCGGTG AGTCCGTCCC TCTAAGTCGT AGTGAGGACG CCGCGGAAGT |
| | E |
| | --------------------- |
| | Y T L K L G E • |
| | TACACACTAA AGCTTGGAGA |
| | ATGTGTGATT TCGAACCTCT |
| | E |
| | ---------------------------------------------------------------------------------------- |
| | • Y G E V T V D C E P R S G I D T N A Y Y V M T V G T K |
| 3101 | ATATGGAGAG GTGACAGTGG ACTGTGAACC ACGGTCAGGG ATTGACACCA ATGCATACTA CGTGATGACT GTTGGAACAA |
| | TATACCTCTC CACTGTCACC TGACACTTGG TGCCAGTCCC TAACTGTGGT TACGTATGAT GCACTACTGA CAACCTTGTT |
| | E |
| | --------------------- |
| | T F L V H R |
| | AGACGTTCTT GGTCCATCGT |
| | TCTGCAAGAA CCAGGTAGCA |
| | E |
| | ---------------------------------------------------------------------------------------- |
| | E W F M D L N L P W S S A G S T V W R N R E T L M E F |
| 3201 | GAGTGGTTCA TGGACCTCAA CCTCCCTTGG AGCAGTGCTG GAAGTACTGT GTGGAGGAAC AGAGAGACGT TAATGGAGTT |
| | CTCACCAAGT ACCTGGAGTT GGAGGGAACC TCGTCACGAC CTTCATGACA CACCTCCTTG TCTCTCTGCA ATTACCTCAA |
| | E |
| | --------------------- |
| | E E P H A T K • |
| | TGAGGAACCA CACGCCACGA |
| | ACTCCTTGGT GTGCGGTGCT |
| | E |
| | ---------------------------------------------------------------------------------------- |
| | • Q S V I A L G S Q E G A L H Q A L A G A I P V E F S |
| 3301 | AGCAGTCTGT GATAGCATTG GGCTCACAAG AGGGAGCTCT GCATCAAGCT TTGGCTGGAG CCATTCCTGT GGAATTTTCA |
| | TCGTCAGACA CTATCGTAAC CCGAGTGTTC TCCCTCGAGA CGTAGTTCGA AACCGACCTC GGTAAGGACA CCTTAAAAGT |
| | E |
| | -------------------- |
| | S N T V K L T • |
| | AGCAACACTG TCAAGTTGAC |
| | TCGTTGTGAC AGTTCAACTG |
| | E |
| | ---------------------------------------------------------------------------------------- |
| | • S G H L K C R V K M E K L Q L K G T T Y G V C S K A F |
| 3401 | GTCGGGTCAT TTGAAGTGTA GAGTGAAGAT GGAAAAATTG CAGTTGAAGG GAACAACCTA TGGCGTCTGT TCAAAGGCTT |
| | CAGCCCAGTA AACTTCACAT CTCACTTCTA CCTTTTTAAC GTCAACTTCC CTTGTTGGAT ACCGCAGACA AGTTTCCGAA |
| | E |
| | --------------------- |
| | K F L G T P |
| | TCAAGTTTCT TGGGACTCCC |
| | AGTTCAAAGA ACCCTGAGGG |
| | E |
| | ---------------------------------------------------------------------------------------- |
| | A D T G H G T V V L E L Q Y T G T D G P C K V P I S S |
| 3501 | GCAGACACAG GTCACGGCAC TGTGGTGTTG GAATTGCAGT ACACTGGCAC GGATGGACCT TGCAAAGTTC CTATCTCGTC |
| | CGTCTGTGTC CAGTGCCGTG ACACCACAAC CTTAACGTCA TGTGACCGTG CCTACCTGGA ACGTTTCAAG GATAGAGCAG |
| | E |
| | --------------------- |
| | V A S L N D L • |
| | AGTGGCTTCA TTGAACGACC |
| | TCACCGAAGT AACTTGCTGG |
| | E |
| | ---------------------------------------------------------------------------------------- |
| | • T P V G R L V T V N P F V S V A T A N A K V L I E L |
| 3601 | TAACGCCAGT GGGCAGATTG GTCACTGTCA ACCCTTTTGT TTCAGTGGCC ACGGCCAACG CTAAGGTCCT GATTGAATTG |
| | ATTGCGGTCA CCCGTCTAAC CAGTGACAGT TGGGAAAACA AAGTCACCGG TGCCGGTTGC GATTCCAGGA CTAACTTAAC |
| | E |
| | --------------------- |
| | E P P F G D S • |
| | GAACCACCCT TTGGAGACTC |
| | CTTGGTGGGA AACCTCTGAG |
| | E |
| | ---------------------------------------------------------------------------------------- |
| | • Y I V V G R G E Q Q I N H H W H K S G S S I G K A F T |
| 3701 | ATACATAGTG GTGGGCAGAG GAGAACAACA GATCAATCAC CACTGGCACA AGTCTGGAAG CAGCATTGGC AAAGCCTTTA |
| | TATGTATCAC CACCCGTCTC CTCTTGTTGT CTAGTTAGTG GTGACCGTGT TCAGACCTTC GTCGTAACCG TTTCGGAAAT |
| | E |
| | --------------------- |
| | T T L K G A |
| | CAACCACCCT CAAAGGAGCG |
| | GTTGGTGGGA GTTTCCTCGC |
| | E |
| | ---------------------------------------------------------------------------------------- |
| | Q R L A A L G D T A W D F G S V G G V F T S V G K A V |
| 3801 | CAGAGACTAG CCGCTCTAGG AGACACAGCT TGGGACTTTG GATCAGTTGG AGGGGTGTTC ACCTCAGTTG GGAAGGCTGT |
| | GTCTCTGATC GGCGAGATCC TCTGTGTCGA ACCCTGAAAC CTAGTCAACC TCCCCACAAG TGGAGTCAAC CCTTCCGACA |
| | E |
| | --------------------- |
| | H Q V F G G A • |
| | CCATCAAGTG TTCGGAGGAG |
| | GGTAGTTCAC AAGCCTCCTC |
| | E |
| | ---------------------------------------------------------------------------------------- |
| | • F R S L F G G M S W I T Q G L L G A L L L W M G I N |
| 3901 | CATTCCGCTC ACTGTTCGGA GGCATGTCCT GGATAACGCA AGGATTGCTG GGGGCTCTCC TGTTGTGGAT GGGCATCAAT |
| | GTAAGGCGAG TGACAAGCCT CCGTACAGGA CCTATTGCGT TCCTAACGAC CCCCGAGAGG ACAACACCTA CCCGTAGTTA |
| | E |
| | --------------------- |
| | A R D R S I A • |
| | GCTCGTGACA GGTCCATAGC |
| | CGAGCACTGT CCAGGTATCG |
| | NS1 |
| | ------------------------- |
| | E |
| | --------------------------------------------------------------- |
| | • L T F L A V G G V L L F L S V N V H A D T G C A I D I |
| 4001 | TCTCACGTTT CTCGCAGTTG GAGGAGTTCT GCTCTTCCTC TCCGTGAACG TGCACGCTGA CACTGGGTGT GCCATAGACA |
| | AGAGTGCAAA GAGCGTCAAC CTCCTCAAGA CGAGAAGGAG AGGCACTTGC ACGTGCGACT GTGACCCACA CGGTATCTGT |
| | NS1 |
| | --------------------- |
| | S R Q E L R |
| | TCAGCCGGCA AGAGCTGAGA |
| | AGTCGGCCGT TCTCGACTCT |

SEQUENCE APPENDIX 6

RepliVax WN - Anchorless F inserted in place of ΔprM-E.
F insert starts at nucleotide position 439 bp and ends at 2016 bp.

| | |
|---|---|
| | 5′ UTR |
| | ---------------------------------------------------------------------------------------- |
| 1 | AGTAGTTCGC CTGTGTGAGC TGACAAACTT AGTAGTGTTT GTGAGGATTA ACAACAATTA ACACAGTGCG AGCTGTTTCT |
| | TCATCAAGCG GACACACTCG ACTGTTTGAA TCATCACAAA CACTCCTAAT TGTTGTTAAT TGTGTCACGC TCGACAAAGA |
| | C |
| | ---- |
| | 5′ UTR |
| | ----------------- |
| | M S |
| | TAGCACGAAG ATCTCGATGT |
| | ATCGTGCTTC TAGAGCTACA |
| | C |
| | ---------------------------------------------------------------------------------------- |
| | • K K P G G P G K S R A V Y L L K R G M P R V L S L I |
| 101 | CTAAGAAACC AGGAGGGCCC GGCAAGAGCC GGGCTGTCTA TTTGCTAAAA CGCGGAATGC CCCGCGTGTT GTCCTTGATT |
| | GATTCTTTGG TCCTCCCGGG CCGTTCTCGG CCCGACAGAT AAACGATTTT GCGCCTTACG GGGCGCACAA CAGGAACTAA |
| | C |
| | --------------------- |
| | G L K R A M L • |
| | GGACTTAAGA GGGCTATGTT |
| | CCTGAATTCT CCCGATACAA |
| | C |
| | ---------------------------------------------------------------------------------------- |
| | • S L I D G K G P I R F V L A L L A F F R F T A I A P T |
| 201 | GAGCCTGATC GACGGCAAGG GGCCAATACG ATTTGTGTTG GCTCTCTTGG CGTTCTTCAG GTTCACAGCA ATTGCTCCGA |
| | CTCGGACTAG CTGCCGTTCC CCGGTTATGC TAAACACAAC CGAGAGAACC GCAAGAAGTC CAAGTGTCGT TAACGAGGCT |
| | C |
| | --------------------- |
| | R A V L D R |
| | CCCGAGCAGT GCTGGATCGA |
| | GGGCTCGTCA CGACCTAGCT |
| | cleavage |
| | C |
| | ---------------------------------------------------------------------------------------- |
| | W R G V N K Q T A M K H L L S F K K E L G T L T S A I |
| 301 | TGGAGAGGTG TGAACAAACA AACAGCGATG AAACACCTTC TGAGTTTCAA GAAGGAACTA GGGACCTTGA CCAGTGCTAT |
| | ACCTCTCCAC ACTTGTTTGT TTGTCGCTAC TTTGTGGAAG ACTCAAAGTT CTTCCTTGAT CCCTGGAACT GGTCACGATA |
| | NS3 |
| | ---- |
| | C |
| | ------------------ |
| | N R R S S K Q |
| | CAATCGGCGG AGCTCAAAGC |
| | GTTAGCCGCC TCGAGTTTCG |
| | Anchorless RSV F |
| | ----------------------------------------------- |
| | NS3 cleavage partial C signal |
| | ------------ ---------------------------- |
| | • K K R G G K T G I A V I M E L P I I K A N A I T T I |
| 401 | AAAAGAAGCG AGGGGGCAAG ACTGGTATAG CTGTGATCAT GGAACTGCCC ATCATCAAGG CCAACGCCAT CACCACCATC |
| | TTTTCTTCGC TCCCCCGTTC TGACCATATC GACACTAGTA CCTTGACGCG TAGTAGTTCC GGTTGCGGTA GTGGTGGTAG |
| | Anchorless RSV F |
| | --------------------- |
| | L I A V T F C • |
| | CTGATCGCCG TGACCTTCTG |
| | GACTAGCGGC ACTGGAAGAC |
| | Ancorless RSV F |
| | ---------------------------------------------------------------------------------------- |
| | • F A S Q N I T E E F Y Q S T C S A V S K G Y L S A L |
| 501 | CTTCGCCAGC AGCCAGAACA TCACCGAGGA ATTCTACCAG AGCACCTGCA GCGCCGTGAG CAAGGGCTAC CTGAGCGCCC |
| | GAAGCGGTCG TCGGTCTTGT AGTGGCTCCT TAAGATGGTC TCGTGGACGT CGCGGCACTC GTTCCCGATG GACTCGCGGG |
| | Anchorless RSV F |
| | --------------------- |
| | R T G W Y T |
| | TGCGGACCGG CTGGTACACC |
| | ACGCCTGGCC GACCATGTGG |
| | Anchorless RSV F |
| | ---------------------------------------------------------------------------------------- |
| | S V I T I E L S N I K E N K C N G T D A K V K L I K Q |
| 601 | AGCGTGATCA CCATCGAGCT GTCCAACATC AAAGAAAACA AGTGCAACGG CACCGACGCC AAGGTGAAAC TGATCAAGCA |
| | TCGCACTAGT GGTAGCTCGA CAGGTTGTAG TTTCTTTTGT TCACGTTGCC GTGGCTGCGG TTCCACTTTG ACTAGTTCGT |
| | Anchorless RSV F |
| | --------------------- |
| | E L D K Y K N |
| | GGAACTGGAC AAGTACAAGA |
| | CCTTGACCTG TTCATGTTCT |
| | Anchorless RSV F |
| | ---------------------------------------------------------------------------------------- |
| | • A V T E L Q L L M Q S T P A A N N R A R R E L P R F |
| 701 | ACGCCGTGAC CGAGCTGCAG CTGCTGATGC AGAGCACCCC TGCCGCCAAC AACCGGGCCA GACGCGAGCT GCCCCGGTTC |
| | TGCGGCACTG GCTCGACGTC GACGACTACG TCTCGTGGGG ACGGCGGTTG TTGGCCCGGT CTGCGCTCGA CGGGGCCAAG |
| | Anchorless RSV F |
| | --------------------- |
| | M N Y T L N N • |
| | ATGAACTACA CCCTGAACAA |
| | TACTTGATGT GGGACTTGTT |
| | Anchorless RSV F |
| | ---------------------------------------------------------------------------------------- |
| | • A K K T N V T L S K K R K R R F L G F L L G V G S A I |
| 801 | CGCCAAGAAA ACCAACGTGA CCCTGAGCAA GAAGCGGAAG CGGCGGTTCC TGGGCTTCCT GCTGGGCGTG GGCAGCGCCA |
| | GCGGTTCTTT TGGTTGCACT GGGACTCGTT CTTCGCCTTC GCCGCCAAGG ACCCGAAGGA CGACCCGCAC CCGTCGCGGT |
| | Anchorless RSV F |
| | --------------------- |
| | A S G I A V |
| | TCGCCAGCGG CATCGCCGTG |
| | AGCGGTCGCC GTAGCGGCAC |
| | Anchorless RSV F |
| | ---------------------------------------------------------------------------------------- |
| | S K V L H L E G E V N K I K S A L L S T N K A V V S L |
| 901 | TCCAAGGTGC TGCACCTGGA AGGCGAGGTG AACAAGATCA AGTCCGCCCT GCTGTCCACC AACAAGGCCG TGGTGTCCCT |
| | AGGTTCCACG ACGTGGACCT TCCGCTCCAC TTGTTCTAGT TCAGGCGGGA CGACAGGTGG TTGTTCCGGC ACCACAGGGA |
| | Anchorless RSV F |
| | --------------------- |
| | S N G V S V L • |
| | GAGCAACGGC GTGAGCGTGC |
| | CTCGTTGCCG CACTCGCACG |
| | Anchorless RSV F |
| | ---------------------------------------------------------------------------------------- |
| | • T S K V L D L K N Y I D K Q L L P I V N K Q S C S I |
| 1001 | TGACCAGCAA GGTGCTGGAT CTGAAGAACT ACATCGACAA GCAGCTGCTG CCCATCGTGA ACAAGCAGAG CTGCAGCATC |
| | ACTGGTCGTT CCACGACCTA GACTTCTTGA TGTAGCTGTT CGTCGACGAC GGGTAGCACT TGTTCGTCTC GACGTCGTAG |
| | Anchorless RSV F |
| | --------------------- |
| | S N I E T V I • |
| | AGCAACATCG AGACCGTGAT |
| | TCGTTGTAGC TCTGGCACTA |
| | Anchorless RSV F |
| | ---------------------------------------------------------------------------------------- |
| | • E F Q Q K N N R L L E I T R E F S V N A G V T T P V S |
| 1101 | CGAGTTCCAG CAGAAGAACA ACCGGCTGCT GGAAATCACC CGGGAGTTCA GCGTGAACGC CGGCGTGACC ACCCCCGTGA |
| | GCTCAAGGTC GTCTTCTTGT TGGCCGACGA CCTTTAGTGG GCCCTCAAGT CGCACTTGCG GCCGCACTGG TGGGGGCACT |
| | Anchorless RSV F |
| | --------------------- |
| | T Y M L T N |
| | GCACCTACAT GCTGACCAAC |
| | CGTGGATGTA CGACTGGTTG |
| | Anchorless RSV F |
| | ---------------------------------------------------------------------------------------- |
| | S E L L S L I N D M P I T N D Q K K L M S N N V Q I V |
| 1201 | AGCGAGCTGC TGTCCCTGAT CAATGACATG CCCATCACCA ACGACCAGAA GAAACTGATG AGCAACAACG TGCAGATCGT |
| | TCGCTCGACG ACAGGGACTA GTTACTGTAC GGGTAGTGGT TGCTGGTCTT CTTTGACTAC TCGTTGTTGC ACGTCTAGCA |
| | Anchorless RSV F |
| | --------------------- |
| | R Q Q S Y S I • |
| | GCGGCAGCAG AGCTACTCCA |
| | CGCCGTCGTC TCGATGAGGT |
| | Anchorless RSV F |
| | ---------------------------------------------------------------------------------------- |
| | • M S I I K E E V L A Y V V Q L P L Y G V I D T P C W |
| 1301 | TCATGAGCAT CATCAAAGAA GAGGTGCTGG CCTACGTGGT GCAGCTGCCC CTGTACGGCG TGATCGACAC CCCCTGCTGG |
| | AGTACTCGTA GTAGTTTCTT CTCCACGACC GGATGCACCA CGTCGACGGG GACATGCCGC ACTAGCTGTG GGGGACGACC |
| | Anchorless RSV F |
| | --------------------- |
| | K L H T S P L • |
| | AAGCTGCACA CCAGCCCCCT |
| | TTCGACGTGT GGTCGGGGGA |
| | Anchorless RSV F |
| | ---------------------------------------------------------------------------------------- |
| | • C T T N T K E G S N I C L T R T D R G W Y C N N A G S |
| 1401 | GTGCACCACC AACACCAAAG AGGGCAGCAA CATCTGCCTG ACCCGGACCG ACCGGGGCTG GTACTGCAAC AACGCCGGCA |
| | CACGTGGTGG TTGTGGTTTC TCCCGTCGTT GTAGACGGAC TGGGCCTGGC TGGCCCCGAC CATGACGTTG TTGCGGCCGT |
| | Anchorless RSV F |
| | --------------------- |
| | V S F F P L |
| | GCGTGAGCTT CTTCCCCCTG |
| | CGCACTCGAA GAAGGGGGAC |
| | Anchorless RSV F |
| | ---------------------------------------------------------------------------------------- |
| | A D T C K V Q S N R V F C D T M N S L T L P S E V N L |
| 1501 | GCCGACACCT GCAAGGTGCA GAGCAACCGG GTGTTCTGCG ACACCATGAA CAGCCTGACC CTGCCCTCCG AGGTGAACCT |
| | CGGCTGTGGA CGTTCCACGT CTCGTTGGCC CACAAGACGC TGTGGTACTT GTCGGACTGG GACGGGAGGC TCCACTTGGA |
| | Anchorless RSV F |
| | --------------------- |
| | C N I D I F N |
| | GTGCAACATC GACATCTTCA |
| | CACGTTGTAG CTGTAGAAGT |
| | Anchorless RSV F |
| | ---------------------------------------------------------------------------------------- |
| | • P K Y D C K I M T S K T D V S S S V I T S L G A I V |
| 1601 | ACCCCAAGTA CGACTGCAAG ATCATGACCT CCAAGACCGA CGTGAGCAGC TCCGTGATCA CCTCCCTGGG CGCCATCGTG |
| | TGGGGTTCAT GCTGACGTTC TAGTACTGGA GGTTCTGGCT GCACTCGTCG AGGCACTAGT GGAGGGACCC GCGGTAGCAC |
| | Anchorless RSV F |
| | --------------------- |
| | S C Y G K T K • |
| | AGCTGCTACG GCAAGACCAA |
| | TCGACGATGC CGTTCTGGTT |
| | Anchorless RSV F |
| | ---------------------------------------------------------------------------------------- |
| | • C T A S N K N R G I I K T F S N G C D Y V S N K G V D |
| 1701 | GTGCACCGCC AGCAACAAGA ACCGGGGCAT CATCAAGACC TTCAGCAACG GCTGCGACTA CGTGAGCAAC AAGGGCGTGG |
| | CACGTGGCGG TCGTTGTTCT TGGCCCCGTA GTAGTTCTGG AAGTCGTTGC CGACGCTGAT GCACTCGTTG TTCCCGCACC |
| | Anchorless RSV F |
| | --------------------- |
| | T V S V G N |
| | ACACCGTGAG CGTGGGCAAC |
| | TGTGGCACTC GCACCCGTTG |
| | Anchorless RSV F |
| | ---------------------------------------------------------------------------------------- |
| | T L Y Y V N K Q E G K S L Y V K G E P I I N F Y D P L |
| 1801 | ACACTGTACT ACGTGAATAA GCAGGAAGGC AAGAGCCTGT ACGTGAAGGG CGAGCCTATC ATCAACTTCT ACGACCCCCT |
| | TGTGACATGA TGCACTTATT CGTCCTTCCG TTCTCGGACA TGCACTTCCC GCTCGGATAG TAGTTGAAGA TGCTGGGGGA |
| | Anchorless RSV F |
| | --------------------- |
| | V F P S D E F • |
| | GGTGTTCCCC AGCGACGAGT |
| | CCACAAGGGG TCGCTGCTCA |
| | Anchorless RSV F |
| | ---------------------------------------------------------------------------------------- |
| | • D A S I S Q V N E K I N Q S L A F I R K S D E L L H |
| 1901 | TCGACGCCAG CATCAGCCAG GTGAACGAGA AGATCAACCA GAGCCTGGCC TTCATCCGGA AGAGCGACGA GCTGCTGCAC |
| | AGCTGCGGTC GTAGTCGGTC CACTTGCTCT TCTAGTTGGT CTCGGACCGG AAGTAGGCCT TCTCGCTGCT CGACGACGTG |
| | Anchorless RSV F |
| | --------------------- |
| | N V N A G K S • |
| | AATGTGAATG CCGGCAAGAG |
| | TTACACTTAC GGCCGTTCTC |
| | pre E/NS1 signal |
| | ---------- |
| | FMDV 2A |
| | -------------------------------------------------------- |
| | Anchorless RSV F |
| | ----------------- |
| | • T T N I M N F D L L K L A G D V E S N P G P A r D |
| 2001 | CACCACCAAT ATCATGAATT TTGATCTGCT CAAACTTGCA GGCGATGTAG AATCAAATCC TGGACCCGCC CGGGAC |
| | GTGGTGGTTA TAGTACTTAA AACTAGACGA GTTTGAACGT CCGCTACATC TTAGTTTAGG ACCTGGGCGG GCCCTG |
| | Transmembrane domain of WNV E (split) |
| | -------------------------- |
| | R S I A L T F L |
| | AGGT CCATAGCTCT CACGTTTCTC |
| | TCCA GGTATCGAGA GTGCAAAGAG |
| | NS1 |
| | --------------------------------------- |
| | Transmembrane domain of WNV E (split) |
| | ------------------------------------------------- |
| | A V G G V L L F L S V N V H A D T G C A I D I S • R Q E |
| 2101 | GCAGTTGGAG GAGTTCTGCT CTTCCTCTCC GTGAACGTGC ACGCTGACAC TGGGTGTGCC ATAGACATCA GCCGGCAAGA |
| | CGTCAACCTC CTCAAGACGA GAAGGAGAGG CACTTGCACG TGCCACTGTG ACCCACACGG TATCTGTAGT CGGCCGTTCT |
| | NS1 |
| | -------------------- |
| | L R C G S G V • |
| | GCTGAGATGT GGAAGTGGAG |
| | CGACTCTACA CCTTCACCTC |
| | NS1 |
| | ---------------------------------------------------------------------------------------- |
| | • F I H N D V E A W M D R Y K Y Y P E T P Q G L A K I |
| 2201 | TGTTCATACA CAATGATGTG GAGGCTTGGA TGGACCGGTA CAAGTATTAC CCTGAAACGC CACAAGGCCT AGCCAAGATC |
| | ACAAGTATGT GTTACTACAC CTCCGAACCT ACCTGGCCAT GTTCATAATG GGACTTTGCG GTGTTCCGGA TCGGTTCTAG |
| | NS1 |
| | --------------------- |
| | I Q K A H K E • |
| | ATTCAGAAAG CTCATAAGGA |
| | TAAGTCTTTC GAGTATTCCT |
| | NS1 |
| | ---------------------------------------------------------------------------------------- |
| | • G V C G L R S V S R L E H Q M W E A V K D E L N T L L |
| 2301 | AGGAGTGTGC GGTCTACGAT CAGTTTCCAG ACTGGAGCAT CAAATGTGGG AAGCAGTGAA GGACGAGCTG AACACTCTTT |
| | TCCTCACACG CCAGATGCTA GTCAAAGGTC TGACCTCGTA GTTTACACCC TTCGTCACTT CCTGCTCGAC TTGTGAGAAA |
| | NS1 |
| | --------------------- |
| | K E N G V D |
| | TGAAGGAGAA TGGTGTGGAC |
| | ACTTCCTCTT ACCACACCTG |
| | NS1 |
| | ---------------------------------------------------------------------------------------- |
| | L S V V V E K Q G G M Y K S A P K R L T A T T E K L E |
| 2401 | CTTAGTGTCG TGGTTGAGAA ACAAGGGGGA ATGTACAAGT CAGCACCTAA ACGCCTCACC GCCACCACGG AAAAATTGGA |
| | GAATCACAGC ACCAACTCTT TGTTCCCCCT TACATGTTCA GTCGTGGATT TGCGGAGTGG CGGTGGTGCC TTTTTAACCT |
| | NS1 |
| | --------------------- |
| | I G W K A W G • |
| | AATTGGCTGG AAGGCCTGGG |
| | TTAACCGACC TTCCGGACCC |
| | NS1 |
| | ---------------------------------------------------------------------------------------- |
| | • K S I L F A P E L A N N T F V V D G P E T K E C P T |
| 2501 | GAAAGAGTAT TTTGTTTGCA CCAGAACTCG CCAACAACAC CTTTGTGGTT GATGGTCCGG AGACCAAGGA ATGTCCGACT |
| | CTTTCTCATA AAACAAACGT GGTCTTGAGC GGTTGTTGTG GAAACACCAA CTACCAGGCC TCTGGTTCCT TACAGGCTGA |
| | NS1 |
| | --------------------- |
| | Q N R A W N S • |
| | CAGAATCGCG CTTGGAATAG |
| | GTCTTAGCGC GAACCTTATC |
| | NS1 |
| | ---------------------------------------------------------------------------------------- |
| | • L E V E D F G F G L T S T R M F L K V R E S N T T E C |
| 2601 | CTTAGAAGTG GAGGATTTTG GATTTGGTCT CACCAGCACT CGGATGTTCC TGAAGGTCAG AGAGAGCAAC ACAACTGAAT |
| | GAATCTTCAC CTCCTAAAAC CTAAACCAGA GTGGTCGTGA GCCTACAAGG ACTTCCAGTC TCTCTCGTTG TGTTGACTTA |
| | NS1 |
| | --------------------- |
| | D S K I I G |
| | GTGACTCGAA GATCATTGGA |
| | CACTGAGCTT CTAGTAACCT |
| | NS1 |
| | ---------------------------------------------------------------------------------------- |
| | T A V K N N L A I H S D L S Y W I E S R L N D T W K L |
| 2701 | ACGGCTGTCA AGAACAACTT GGCGATCCAC AGTGACCTGT CCTATTGGAT TGAAAGCAGG CTCAATGATA CGTGGAAGCT |
| | TGCCGACAGT TCTTGTTGAA CCGCTAGGTG TCACTGGACA GGATAACCTA ACTTTCGTCC GAGTTACTAT GCACCTTCGA |
| | NS1 |
| | --------------------- |
| | E R A V L G E • |
| | TGAAAGGGCA GTTCTGGGTG |
| | ACTTTCCCGT CAAGACCCAC |
| | NS1 |
| | ---------------------------------------------------------------------------------------- |
| | • V K S C T W P E T H T L W G D G I L E S D L I I P V |
| 2801 | AAGTCAAATC ATGTACGTGG CCTGAGACGC ATACCTTGTG GGGCGATGGA ATCCTTGAGA GTGACTTGAT AATACCAGTC |
| | TTCAGTTTAG TACATGCACC GGACTCTGCG TATGGAACAC CCCGCTACCT TAGGAACTCT CACTGAACTA TTATGGTCAG |
| | NS1 |
| | --------------------- |
| | T L A G P R S • |
| | ACACTGGCGG GACCACGAAG |
| | TGTGACCGCC CTGGTGCTTC |
| | NS1 |
| | ---------------------------------------------------------------------------------------- |
| | • N H N R R P G Y K T Q N Q G P W D E G R V E I D F D Y |
| 2901 | CAATCACAAT CGGAGACCTG GGTATAAGAC ACAAAACCAG GGCCCATGGG ACGAAGGCCG GGTAGAGATT GACTTCGATT |
| | GTTAGTGTTA GCCTCTGGAC CCATATTCTG TGTTTTGGTC CCGGGTACCC TGCTTCCGGC CCATCTCTAA CTGAAGCTAA |
| | NS1 |
| | --------------------- |
| | C P G T T V |
| | ACTGCCCAGG AACTACGGTC |
| | TGACGGGTCC TTGATGCCAG |
| | NS1 |
| | ---------------------------------------------------------------------------------------- |
| | T L S E S C G H R G P A T R T T T E S G K L I T D W C |
| 3001 | ACCCTGAGTG AGAGCTGCGG ACACCGTGGA CCTGCCACTC GCACCACCAC AGAGAGCGGA AAGTTGATAA CAGATTGGTG |
| | TGGGACTCAC TCTCGACGCC TGTGGCACCT GGACGGTGAG CGTGGTGGTG TCTCTCGCCT TTCAACTATT GTCTAACCAC |
| | NS1 |
| | --------------------- |
| | C R S C T L P • |
| | CTGCAGGAGC TGCACCTTAC |
| | GACGTCCTCG ACGTGGAATG |
| | NS1 |
| | ---------------------------------------------------------------------------------------- |
| | • P L R Y Q T D S G C W Y G M E I R P Q R H D E K T L |
| 3101 | CACCACTGCG CTACCAAACT GACAGCGGCT GTTGGTATGG TATGGAGATC AGACCACAGA GACATGATGA AAAGACCCTC |
| | GTGGTGACGC GATGGTTTGA CTGTCGCCGA CAACCATACC ATACCTCTAG TCTGGTGTCT CTGTACTACT TTTCTGGGAG |
| | NS1 |
| | --------------------- |
| | V Q S Q V N A • |
| | GTGCAGTCAC AAGTGAATGC |
| | CACGTCAGTG TTCACTTACG |
| | NS1 |
| | - |
| | NS2A |
| | --------------------------------------------------------------------------------------- |
| | • Y N A D M I D P F Q L G L L V V F L A T Q E V L R K R |
| 3201 | TTATAATGCT GATATGATTG ACCCTTTTCA GTTGGGCCTT CTGGTCGTGT TCTTGGCCAC CCAGGAGGTC CTTCGCAAGA |
| | AATATTACGA CTATACTAAC TGGGAAAAGT CAACCCGGAA GACCAGCACA AGAACCGGTG GGTCCTCCAG GAAGCGTTCT |
| | NS2A |
| | --------------------- |
| | W T A K I S |
| | GGTGGACAGC CAAGATCAGC |
| | CCACCTGTCG GTTCTAGTCG |
| | NS2A |
| | ---------------------------------------------------------------------------------------- |
| | M P A I L I A L L V L V F G G I T Y T D V L R Y V I L |
| 3301 | ATGCCAGCTA TACTGATTGC TCTGCTAGTC CTGGTGTTTG GGGGCATTAC TTACACTGAT GTGTTACGCT ATGTCATCTT |
| | TACGGTCGAT ATGACTAACG AGACGATCAG GACCACAAAC CCCCGTAATG AATGTGACTA CACAATGCGA TACAGTAGAA |
| | NS2A |
| | --------------------- |
| | V G A A F A E • |
| | GGTGGGGGCA GCTTTCGCAG |
| | CCACCCCCGT CGAAAGCGTC |
| | NS2A |
| | ---------------------------------------------------------------------------------------- |
| | • S N S G G D V V H L A L M A T F K I Q P V F M V A S |
| 3401 | AATCTAATTC GGGAGGAGAC GTGGTACACT TGGCGCTCAT GGCGACCTTC AAGATACAAC CAGTGTTTAT GGTGGCATCG |
| | TTAGATTAAG CCCTCCTCTG CACCATGTGA ACCGCGAGTA CCGCTGGAAG TTCTATGTTG GTCACAAATA CCACCGTAGC |
| | NS2A |
| | --------------------- |
| | F L K A R W T • |
| | TTTCTTAAAG CGAGATGGAC |
| | AAAGAATTTC GCTCTACCTG |
| | NS2A |
| | ---------------------------------------------------------------------------------------- |
| | • N Q E N I L L M L A A V F F Q M A Y H D A R Q I L L W |
| 3501 | CAACCAGGAG AACATTTTGT TGATGTTGGC GGCTGTTTTC TTTCAAATGG CTTATCACGA TGCCCGCCAA ATTCTGCTCT |
| | GTTGGTCCTC TTGTAAAACA ACTACAACCG CCGACAAAAG AAAGTTTACC GAATAGTGCT ACGGGCGGTT TAAGACGAGA |
| | NS2A |
| | --------------------- |
| | E I P D V L |
| | GGGAGATCCC TGATGTGTTG |
| | CCCTCTAGGG ACTACACAAC |
| | NS2A |
| | ---------------------------------------------------------------------------------------- |
| | N S L A I A W M I L R A I T F T T T S N V V V P L L A |
| 3601 | AATTCACTGG CAATAGCTTG GATGATACTG AGAGCCATAA CATTCACAAC GACATCAAAC GTGGTTGTTC CGCTGCTAGC |
| | TTAAGTGACC GTTATCGAAC CTACTATGAC TCTCGGTATT GTAAGTGTTG CTGTAGTTTG CACCAACAAG GCGACGATCG |
| | NS2A |
| | --------------------- |
| | L L T P G L R • |
| | CCTGCTAACA CCCGGGCTGA |
| | GGACGATTGT GGGCCCGACT |
| | NS2A |
| | ---------------------------------------------------------------------------------------- |
| | • C L N L D V Y R I L L L M V G I G S L I R E K R S A |
| 3701 | GATGCTTGAA TCTGGATGTG TACAGGATAC TGCTGTTGAT GGTCGGAATA GGCAGCTTGA TCAGGGAGAA GAGGAGCGCA |
| | CTACGAACTT AGACCTACAC ATGTCCTATG ACGACAACTA CCAGCCTTAT CCGTCGAACT AGTCCCTCTT CTCCTCGCGT |
| | NS2A |
| | --------------------- |
| | A A K K K G A • |
| | GCTGCAAAAA AGAAAGGAGC |
| | CGACGTTTTT TCTTTCCTCG |
| | NS2A |
| | ---------------------------------------------------------------------------------------- |
| | • S L L C L A L A S T G L F N P M I L A A G L I A C D P |
| 3801 | AAGTCTGCTA TGCTTGGCTC TAGCCTCAAC AGGACTCTTC AACCCCATGA TCCTTGCTGC TGGACTGATT GCATGTGATC |
| | TTCAGACGAT ACGAACCGAG ATCGGAGTTG TCCTGAGAAG TTGGGGTACT AGGAACGACG ACCTGACTAA CGTACACTAG |
| | NS2B |
| | ------ |
| | NS2A |
| | --------------- |
| | N R K R G W |
| | CCAACCGTAA ACGCGGGTGG |
| | GGTTGGCATT TGCGCCCACC |
| | NS2B |
| | ---------------------------------------------------------------------------------------- |
| | P A T E V M T A V G L M F A I V G G L A E L D I D S M |
| 3901 | CCCGCAACTG AAGTGATGAC AGCTGTCGGC CTAATGTTTG CCATCGTCGG AGGGCTGGCA GAGCTTGACA TTGACTCCAT |
| | GGGCGTTGAC TTCACTACTG TCGACAGCCG GATTACAAAC GGTAGCAGCC TCCCGACCGT CTCGAACTGT AACTGAGGTA |
| | NS2B |
| | --------------------- |
| | A I P M T I A • |
| | GGCCATTCCA ATGACTATCG |
| | CCGGTAAGGT TACTGATAGC |
| | NS2B |
| | ---------------------------------------------------------------------------------------- |
| | • G L M F A A F V I S G K S T D M W I E R T A D I S W |
| 4001 | CGGGGCTCAT GTTTGCTGCT TTCGTGATTT CTGGGAAATC AACAGATATG TGGATTGAGA GAACGGCGGA CATTTCCTGG |
| | GCCCCGAGTA CAAACGACGA AAGCACTAAA GACCCTTTAG TTGTCTATAC ACCTAACTCT CTTGCCGCCT GTAAAGGACC |
| | NS2B |
| | --------------------- |
| | E S D A E I T • |
| | GAAAGTGATG CAGAGATTAC |
| | CTTTCACTAC GTCTCTAATG |
| | NS2B |
| | ---------------------------------------------------------------------------------------- |
| | • G S S E R V D V R L D D D G N F Q L M N D P G A P W K |
| 4101 | AGGCTCGAGC GAAAGAGTTG ATGTGCGGCT TGATGATGAT GGAAACTTCC AGCTCATGAA TGATCCAGGA GCACCTTGGA |
| | TCCGAGCTCG CTTTCTCAAC TACACGCCGA ACTACTACTA CCTTTGAAGG TCGAGTACTT ACTAGGTCCT CGTGGAACCT |
| | NS2B |
| | --------------------- |
| | I W M L R M |
| | AGATATGGAT GCTCAGAATG |
| | TCTATACCTA CGAGTCTTAC |
| | NS2B |
| | ---------------------------------------------------------------------------------------- |
| | V C L A I S A Y T P W A I L P S V V G F W I T L Q Y T |
| 4201 | GTCTGTCTCG CGATTAGTGC GTACACCCCC TGGGCAATCT TGCCCTCAGT AGTTGGATTT TGGATAACTC TCCAATACAC |
| | CAGACAGAGC GCTAATCACG CATGTGGGGG ACCCGTTAGA ACGGGAGTCA TCAACCTAAA ACCTATTGAG AGGTTATGTG |
| | NS3 |
| | -------------- |
| | NS2B |
| | ------- |
| | K R G G V L W • |
| | AAAGAGAGGA GGCGTGTTGT |
| | TTTCTCTCCT CCGCACAACA |
| | NS3 |
| | ---------------------------------------------------------------------------------------- |
| | • D T P S P K E Y K K G D T T T G V Y R I M T R G L L |
| 4301 | GGGACACTCC CTCACCAAAG GAGTACAAAA AGGGGGACAC GACCACCGGC GTCTACAGGA TCATGACTCG TGGGCTGCTC |
| | CCCTGTGAGG GAGTGGTTTC CTCATGTTTT TCCCCCTGTG CTGGTGGCCG CAGATGTCCT AGTACTGAGC ACCCGACGAG |
| | NS3 |
| | --------------------- |
| | G S Y Q A G A • |
| | GGCAGTTATC AAGCAGGAGC |
| | CCGTCAATAG TTCGTCCTCG |
| | NS3 |
| | ---------------------------------------------------------------------------------------- |
| | • G V M V E G V F H T L W H T T K G A A L M S G E G R L |
| 4401 | AGGCGTGATG GTTGAAGGTG TTTTCCACAC CCTTTGGCAT ACAACAAAAG GAGCCGCTTT GATGAGCGGA GAGGGCCGCC |
| | TCCGCACTAC CAACTTCCAC AAAAGGTGTG GGAAACCGTA TGTTGTTTTC CTCGGCGAAA CTACTCGCCT CTCCCGGCGG |
| | NS3 |
| | --------------------- |
| | D P Y W G S |
| | TGGACCCATA CTGGGGCAGT |
| | ACCTGGGTAT GACCCCGTCA |
| | NS3 |
| | ---------------------------------------------------------------------------------------- |
| | V K E D R L C Y G G P W K L Q H K W N G Q D E V Q M I |
| 4501 | GTCAAGGAGG ATCGACTTTG TTACGGAGGA CCCTGGAAAT TGCAGCACAA GTGGAACGGG CAGGATGAGG TGCAGATGAT |
| | CAGTTCCTCC TAGCTGAAAC AATGCCTCCT GGGACCTTTA ACGTCGTGTT CACCTTGCCC GTCCTACTCC ACGTCTACTA |
| | NS3 |
| | --------------------- |
| | V V E P G K N • |
| | TGTGGTGGAA CCTGGCAAGA |
| | ACACCACCTT GGACCGTTCT |
| | NS3 |
| | ---------------------------------------------------------------------------------------- |
| | • V K N V Q T K P G V F K T P E G E I G A V T L D F P |
| 4601 | ACGTTAAGAA CGTCCAGACG AAACCAGGGG TGTTCAAAAC ACCTGAAGGA GAAATCGGGG CCGTGACTTT GGACTTCCCC |
| | TGCAATTCTT GCAGGTCTGC TTTGGTCCCC ACAAGTTTTG TGGACTTCCT CTTTAGCCCC GGCACTGAAA CCTGAAGGGG |
| | NS3 |
| | --------------------- |
| | T G T S G S P • |
| | ACTGGAACAT CAGGCTCACC |
| | TGACCTTGTA GTCCGAGTGG |
| | NS3 |
| | ---------------------------------------------------------------------------------------- |
| | • I V D K N G D V I G L Y G N G V I M P N G S Y I S A I |
| 4701 | AATAGTGGAC AAAAACGGTG ATGTGATTGG GCTTTATGGC AATGGAGTCA TAATGCCCAA CGGCTCATAC ATAAGCGCGA |
| | TTATCACCTG TTTTTGCCAC TACACTAACC CGAAATACCG TTACCTCAGT ATTACGGGTT GCCGAGTATG TATTCGCGCT |
| | NS3 |
| | --------------------- |
| | V Q G E R M |
| | TAGTGCAGGG TGAAAGGATG |
| | ATCACGTCCC ACTTTCCTAC |
| | NS3 |
| | ---------------------------------------------------------------------------------------- |
| | D E P I P A G F E P E M L R K K Q I T V L D L H P G A |
| 4801 | GATGAGCCAA TCCCAGCCGG ATTCGAACCT GAGATGCTGA GGAAAAAACA GATCACTGTA CTGGATCTCC ATCCCGGCGC |
| | CTACTCGGTT AGGGTCGGCC TAAGCTTGGA CTCTACGACT CCTTTTTTGT CTAGTGACAT GACCTAGAGG TAGGGCCGCG |
| | NS3 |
| | --------------------- |
| | G K T R R I L • |
| | CGGTAAAACA AGGAGGATTC |
| | GCCATTTTGT TCCTCCTAAG |
| | NS3 |
| | ---------------------------------------------------------------------------------------- |
| | • P Q I I K E A I N R R L R T A V L A P T R V V A A E |
| 4901 | TGCCACAGAT CATCAAAGAG GCCATAAACA GAAGACTGAG AACAGCCGTG CTAGCACCAA CCAGGGTTGT GGCTGCTGAG |
| | ACGGTGTCTA GTAGTTTCTC CGGTATTTGT CTTCTGACTC TTGTCGGCAC GATCGTGGTT GGTCCCAACA CCGACGACTC |
| | NS3 |
| | --------------------- |
| | M A E A L R G • |
| | ATGGCTGAAG CACTGAGAGG |
| | TACCGACTTC GTGACTCTCC |
| | NS3 |
| | ---------------------------------------------------------------------------------------- |
| | • L P I R Y Q T S A V P R E H N G N E I V D V M C H A T |
| 5001 | ACTGCCCATC CGGTACCAGA CATCCGCAGT GCCCAGAGAA CATAATGGAA ATGAGATTGT TGATGTCATG TGTCATGCTA |
| | TGACGGGTAG GCCATGGTCT GTAGGCGTCA CGGGTCTCTT GTATTACCTT TACTCTAACA ACTACAGTAC ACAGTACGAT |
| | NS3 |
| | --------------------- |
| | L T H R L M |
| | CCCTCACCCA CAGGCTGATG |
| | GGGAGTGGGT GTCCGACTAC |
| | NS3 |
| | ---------------------------------------------------------------------------------------- |
| | S P H R V P N Y N L F V M D E A H F T D P A S I A A R |
| 5101 | TCTCCTCACA GGGTGCCGAA CTACAACCTG TTCGTGATGG ATGAGGCTCA TTTCACCGAC CCAGCTAGCA TTGCAGCAAG |
| | AGAGGAGTGT CCCACGGCTT GATGTTGGAC AAGCACTACC TACTCCGAGT AAAGTGGCTG GGTCGATCGT AACGTCGTTC |
| | NS3 |
| | --------------------- |
| | G Y I S T K V • |
| | AGGTTACATT TCCACAAAGG |
| | TCCAATGTAA AGGTGTTTCC |
| | NS3 |
| | ---------------------------------------------------------------------------------------- |
| | • E L G E A A A I F M T A T P P G T S D P F P E S N S |
| 5201 | TCGAGCTAGG GGAGGCGGCG GCAATATTCA TGACAGCCAC CCCACCAGGC ACTTCAGATC CATTCCCAGA GTCCAATTCA |
| | AGCTCGATCC CCTCCGCCGC CGTTATAAGT ACTGTCGGTG GGGTGGTCCG TGAAGTCTAG GTAAGGGTCT CAGGTTAAGT |
| | NS3 |
| | --------------------- |
| | P I S D L Q T • |
| | CCAATTTCCG ACTTACAGAC |
| | GGTTAAAGGC TGAATGTCTG |
| | NS3 |
| | ---------------------------------------------------------------------------------------- |
| | • E I P D R A W N S G Y E W I T E Y T G K T V W F V P S |
| 5301 | TGAGATCCCG GATCGAGCTT GGAACTCTGG ATACGAATGG ATCACAGAAT ACACCGGGAA GACGGTTTGG TTTGTGCCTA |
| | ACTCTAGGGC CTAGCTCGAA CCTTGAGACC TATGCTTACC TAGTGTCTTA TGTGGCCCTT CTGCCAAACC AAACACGGAT |
| | NS3 |
| | --------------------- |
| | V K M G N E |
| | GTGTTAAGAT GGGGAATGAG |
| | CACAATTCTA CCCCTTACTC |
| | NS3 |
| | ---------------------------------------------------------------------------------------- |
| | I A L C L Q R A G K K V V Q L N R K S Y E T E Y P K C |
| 5401 | ATTGCCCTTT GCCTACAACG TGCTGGAAAG AAAGTAGTCC AATTGAACAG AAAGTCGTAC GAGACGGAGT ACCCAAAATG |
| | TAACGGGAAA CGGATGTTGC ACGACCTTTC TTTCATCAGG TTAACTTGTC TTTCAGCATG CTCTGCCTCA TGGGTTTTAC |
| | NS3 |
| | --------------------- |
| | K N D D W D F • |
| | TAAGAACGAT GATTGGGACT |
| | ATTCTTGCTA CTAACCCTGA |
| | NS3 |
| | ---------------------------------------------------------------------------------------- |
| | • V I T T D I S E M G A N F K A S R V I D S R K S V K |
| 5501 | TTGTTATCAC AACAGACATA TCTGAAATGG GGGCTAACTT CAAGGCGAGC AGGGTGATTG ACAGCCGGAA GAGTGTGAAA |
| | AACAATAGTG TTGTCTGTAT AGACTTTACC CCCGATTGAA GTTCCGCTCG TCCCACTAAC TGTCGGCCTT CTCACACTTT |
| | NS3 |
| | --------------------- |
| | P T I I T E G • |
| | CCAACCATCA TAACAGAAGG |
| | GGTTGGTAGT ATTGTCTTCC |
| | NS3 |
| | ---------------------------------------------------------------------------------------- |
| | • E G R V I L G E P S A V T A A S A A Q R R G R I G R N |
| 5601 | AGAAGGGAGA GTGATCCTGG GAGAACCATC TGCAGTGACA GCAGCTAGTG CCGCCCAGAG ACGTGGACGT ATCGGTAGAA |
| | TCTTCCCTCT CACTAGGACC CTCTTGGTAG ACGTCACTGT CGTCGATCAC GGCGGGTCTC TGCACCTGCA TAGCCATCTT |
| | NS3 |
| | --------------------- |
| | P S Q V G D |
| | ATCCGTCGCA AGTTGGTGAT |
| | TAGGCAGCGT TCAACCACTA |
| | NS3 |
| | ---------------------------------------------------------------------------------------- |
| | E Y C Y G G H T N E D D S N F A H W T E A R I M L D N |
| 5701 | GAGTACTGTT ATGGGGGGCA CACGAATGAA GACGACTCGA ACTTCGCCCA TTGGACTGAG GCACGAATCA TGCTGGACAA |
| | CTCATGACAA TACCCCCCGT GTGCTTACTT CTGCTGAGCT TGAAGCGGGT AACCTGACTC CGTGCTTAGT ACGACCTGTT |
| | NS3 |
| | --------------------- |
| | I N M P N G L • |
| | CATCAACATG CCAAACGGAC |
| | GTAGTTGTAC GGTTTGCCTG |
| | NS3 |
| | ---------------------------------------------------------------------------------------- |
| | • I A Q F Y Q P E R E K V Y T M D G E Y R L R G E E R |
| 5801 | TGATCGCTCA ATTCTACCAA CCAGAGCGTG AGAAGGTATA TACCATGGAT GGGGAATACC GGCTCAGAGG AGAAGAGAGA |
| | ACTAGCGAGT TAAGATGGTT GGTCTCGCAC TCTTCCATAT ATGGTACCTA CCCCTTATGG CCGAGTCTCC TCTTCTCTCT |
| | NS3 |
| | --------------------- |
| | K N F L E L L • |
| | AAAAACTTTC TGGAACTGTT |
| | TTTTTGAAAG ACCTTGACAA |
| | NS3 |
| | ---------------------------------------------------------------------------------------- |
| | • R T A D L P V W L A Y K V A A A G V S Y H D R R W C F |
| 5901 | GAGGACTGCA GATCTGCCAG TTTGGCTGGC TTACAAGGTT GCAGCGGCTG GAGTGTCATA CCACGACCGG AGGTGGTGCT |
| | CTCCTGACGT CTAGACGGTC AAACCGACCG AATGTTCCAA CGTCGCCGAC CTCACAGTAT GGTGCTGGCC TCCACCACGA |
| | NS3 |
| | --------------------- |
| | D G P R T N |
| | TTGATGGTCC TAGGACAAAC |
| | AACTACCAGG ATCCTGTTTG |
| | NS3 |
| | ---------------------------------------------------------------------------------------- |
| | T I L E D N N E V E V I T K L G E R K I L R P R W I D |
| 6001 | ACAATTTTAG AAGACAACAA CGAAGTGGAA GTCATCACGA AGCTTGGTGA AAGGAAGATT CTGAGGCCGC GCTGGATTGA |
| | TGTTAAAATC TTCTGTTGTT GCTTCACCTT CAGTAGTGCT TCGAACCACT TTCCTTCTAA GACTCCGGCG CGACCTAACT |
| | NS3 |
| | --------------------- |
| | A R V Y S D H • |
| | CGCCAGGGTG TACTCGGATC |
| | GCGGTCCCAC ATGAGCCTAG |
| | NS4A |
| | ---------------------------------------- |
| | NS3 |
| | ------------------------------------------------ |
| | • Q A L K A F K D F A S G K R S Q I G L I E V L G K M |
| 6101 | ACCAGGCACT AAAGGCGTTC AAGGACTTCG CCTCGGGAAA ACGTTCTCAG ATAGGGCTCA TTGAGGTTCT GGGAAAGATG |
| | TGGTCCGTGA TTTCCGCAAG TTCCTGAAGC GGAGCCCTTT TGCAAGAGTC TATCCCGAGT AACTCCAAGA CCCTTTCTAC |
| | NS4A |
| | --------------------- |
| | P E H F M G K • |
| | CCTGAGCACT TCATGGGGAA |
| | GGACTCGTGA AGTACCCCTT |
| | NS4A |
| | ---------------------------------------------------------------------------------------- |
| | • T W E A L D T M Y V V A T A E K G G R A H R M A L E E |
| 6201 | GACATGGGAA GCACTTGACA CCATGTACGT TGTGGCCACT GCAGAGAAAG GAGGAAGAGC TCACAGAATG GCCCTGGAGG |
| | CTGTACCCTT CGTGAACTGT GGTACATGCA ACACCGGTGA CGTCTCTTTC CTCCTTCTCG AGTGTCTTAC CGGGACCTCC |
| | NS4A |
| | --------------------- |
| | L P D A L Q |
| | AACTGCCAGA TGCTCTTCAG |
| | TTGACGGTCT ACGAGAAGTC |
| | NS4A |
| | ---------------------------------------------------------------------------------------- |
| | T I A L I A L L S V M T M G V F F L L M Q R K G I G K |
| 6301 | ACAATTGCCT TGATTGCCTT ATTGAGTGTG ATGACCATGG GAGTATTCTT CCTCCTCATG CAGCGGAAGG GCATTGGAAA |
| | TGTTAACGGA ACTAACGGAA TAACTCACAC TACTGGTACC CTCATAAGAA GGAGGAGTAC GTCGCCTTCC CGTAACCTTT |
| | NS4A |
| | --------------------- |
| | I G L G G A V • |
| | GATAGGTTTG GGAGGCGCTG |
| | CTATCCAAAC CCTCCGCGAC |
| | NS4A |
| | ---------------------------------------------------------------------------------------- |
| | • L G V A T F F C W M A E V P G T K I A G M L L L S L |
| 6401 | TCTTGGGAGT CGCGACCTTT TTCTGTTGGA TGGCTGAAGT TCCAGGAACG AAGATCGCCG GAATGTTGCT GCTCTCCCTT |
| | AGAACCCTCA GCGCTGGAAA AAGACAACCT ACCGACTTCA AGGTCCTTGC TTCTAGCGGC CTTACAACGA CGAGAGGGAA |
| | NS4A |
| | --------------------- |
| | L L M I V L I • |
| | CTCTTGATGA TTGTGCTAAT |
| | GAGAACTACT AACACGATTA |
| | NS4A |
| | ---------------------------------------------------------------------------------------- |
| | • P E P E K Q R S Q T D N Q L A V F L I C V M T L V S A |
| 6501 | TCCTGAGCCA GAGAAGCAAC GTTCGCAGAC AGACAACCAG CTAGCCGTGT TCCTGATATG TGTCATGACC CTTGTGAGCG |
| | AGGACTCGGT CTCTTCGTTG CAAGCGTCTG TCTGTTGGTC GATCGGCACA AGGACTATAC ACAGTACTGG GAACACTCGC |
| | NS4B |
| | --------- |
| | NS4A |
| | ------------ |
| | V A A N E M |
| | CAGTGGCAGC CAACGAGATG |
| | GTCACCGTCG GTTGCTCTAC |
| | NS4B |
| | ---------------------------------------------------------------------------------------- |
| | G W L D K T K S D I S S L F G Q R I E V K E N F S M G |
| 6601 | GGTTGGCTAG ATAAGACCAA GAGTGACATA AGCAGTTTGT TTGGGCAAAG AATTGAGGTC AAGGAGAATT TCAGCATGGG |
| | CCAACCGATC TATTCTGGTT CTCACTGTAT TCGTCAAACA AACCCGTTTC TTAACTCCAG TTCCTCTTAA AGTCGTACCC |
| | NS4B |
| | --------------------- |
| | E F L L D L R • |
| | AGAGTTTCTT CTGGACTTGA |
| | TCTCAAAGAA GACCTGAACT |
| | NS4B |
| | ---------------------------------------------------------------------------------------- |
| | • P A T A W S L Y A V T T A V L T P L L K H L I T S D |
| 6701 | GGCCGGCAAC AGCCTGGTCA CTGTACGCTG TGACAACAGC GGTCCTCACT CCACTGCTAA AGCATTTGAT CACGTCAGAT |
| | CCGGCCGTTG TCGGACCAGT GACATGCGAC ACTGTTGTCG CCAGGAGTGA GGTGACGATT TCGTAAACTA GTGCAGTCTA |
| | NS4B |
| | --------------------- |
| | Y I N T S L T • |
| | TACATCAACA CCTCATTGAC |
| | ATGTAGTTGT GGAGTAACTG |
| | NS4B |
| | ---------------------------------------------------------------------------------------- |
| | • S I N V Q A S A L F T L A R G F P F V D V G V S A L L |
| 6801 | CTCAATAAAC GTTCAGGCAA GTGCACTATT CACACTCGCG CGAGGCTTCC CCTTCGTCGA TGTTGGAGTG TCGGCTCTCC |
| | GAGTTATTTG CAAGTCCGTT CACGTGATAA GTGTGAGCGC GCTCCGAAGG GGAAGCAGCT ACAACCTCAC AGCCGAGAGG |
| | NS4B |
| | --------------------- |
| | L A A G C W |
| | TGCTAGCAGC CGGATGCTGG |
| | ACGATCGTCG GCCTACGACC |
| | NS4B |
| | ---------------------------------------------------------------------------------------- |
| | G Q V T L T V T V T A A T L L F C H Y A Y M V P G W Q |
| 6901 | GGACAAGTCA CCCTCACCGT TACGGTAACA GCGGCAACAC TCCTTTTTTG CCACTATGCC TACATGGTTC CCGGTTGGCA |
| | CCTGTTCAGT GGGAGTGGCA ATGCCATTGT CGCCGTTGTG AGGAAAAAAC GGTGATACGG ATGTACCAAG GGCCAACCGT |
| | NS4B |
| | --------------------- |
| | A E A M R S A • |
| | AGCTGAGGCA ATGCGCTCAG |
| | TCGACTCCGT TACGCGAGTC |
| | NS4B |
| | ---------------------------------------------------------------------------------------- |
| | • Q R R T A A G I M K N A V V D G I V A T D V P E L E |
| 7001 | CCCAGCGGCG GACAGCGGCC GGAATCATGA AGAACGCTGT AGTGGATGGC ATCGTGGCCA CGGACGTCCC AGAATTAGAG |
| | GGGTCGCCGC CTGTCGCCGG CCTTAGTACT TCTTGCGACA TCACCTACCG TAGCACCGGT GCCTGCAGGG TCTTAATCTC |
| | NS4B |
| | --------------------- |
| | R T T P I M Q • |
| | CGCACCACAC CCATCATGCA |
| | GCGTGGTGTG GGTAGTACGT |
| | NS4B |
| | ---------------------------------------------------------------------------------------- |
| | • K K I G Q I M L I L V S L A A V V V N P S V K T V R E |
| 7101 | GAAGAAAATT GGACAGATCA TGCTGATCTT GGTGTCTCTA GCTGCAGTAG TAGTGAACCC GTCTGTGAAG ACAGTACGAG |
| | CTTCTTTTAA CCTGTCTAGT ACGACTAGAA CCACAGAGAT CGACGTCATC ATCACTTGGG CAGACACTTC TGTCATGCTC |
| | NS4B |
| | --------------------- |
| | A G I L I T |
| | AAGCCGGAAT TTTGATCACG |
| | TTCGGCCTTA AAACTAGTGC |
| | NS4B |
| | ---------------------------------------------------------------------------------------- |
| | A A A V T L W E N G A S S V W N A T T A I G L C H I M |
| 7201 | GCCGCAGCGG TGACGCTTTG GGAGAATGGA GCAAGCTCTG TTTGGAACGC AACAACTGCC ATCGGACTCT GCCACATCAT |
| | CGGCGTCGCC ACTGCGAAAC CCTCTTACCT CGTTCGAGAC AAACCTTGCG TTGTTGACGG TAGCCTGAGA CGGTGTAGTA |
| | NS4B |
| | --------------------- |
| | R G G W L S C • |
| | GCGTGGGGGT TGGTTGTCAT |
| | CGCACCCCCA ACCAACAGTA |
| | NS5 |
| | -------------------------- |
| | NS4B |
| | ------------------------------------------------------------- |
| | • L S I T W T L I K N M E K P G L K R G G A K G R T L |
| 7301 | GTCTATCCAT AACATGGACA CTCATAAAGA ACATGGAAAA ACCAGGACTA AAAAGAGGTG GGGCAAAAGG ACGCACCTTG |
| | CAGATAGGTA TTGTACCTGT GAGTATTTCT TGTACCTTTT TGGTCCTGAT TTTTCTCCAC CCCGTTTTCC TGCGTGGAAC |
| | NS5 |
| | --------------------- |
| | G E V W K E R • |
| | GGAGAGGTTT GGAAAGAAAG |
| | CCTCTCCAAA CCTTTCTTTC |
| | NS5 |
| | ---------------------------------------------------------------------------------------- |
| | • L N Q M T K E E F T R Y R K E A I E V D R S A A K H |
| 7401 | ACTCAACCAG ATGACAAAAG AAGAGTTCAC TAGGTACCGC AAAGAGGCCA TCATCGAAGT CGATCGCTCA GCGGCAAAAC |
| | TGAGTTGGTC TACTGTTTTC TTCTCAAGTG ATCCATGGCG TTTCTCCGGT AGTAGCTTCA GCTAGCGAGT CGCCGTTTTG |
| | NS5 |
| | --------------------- |
| | A R K E G N |
| | ACGCCAGGAA AGAAGGCAAT |
| | TGCGGTCCTT TCTTCCGTTA |
| | NS5 |
| | ---------------------------------------------------------------------------------------- |
| | V T G G H P V S R G T A K L R W L V E R R F L E P V G |
| 7501 | GTCACTGGAG GGCATCCAGT CTCTAGGGGC ACAGCAAAAC TGAGATGGCT GGTCGAACGG AGGTTTCTCG AACCGGTCGG |
| | CAGTGACCTC CCGTAGGTCA GAGATCCCCG TGTCGTTTTG ACTCTACCGA CCAGCTTGCC TCCAAAGAGC TTGGCCAGCC |
| | NS5 |
| | --------------------- |
| | K V I D L G C • |
| | AAAAGTGATT GACCTTGGAT |
| | TTTTCACTAA CTGGAACCTA |
| | NS5 |
| | ---------------------------------------------------------------------------------------- |
| | • G R G G W C Y Y M A T Q K R V Q E V R G Y T K G G P |
| 7601 | GTGGAAGAGG CGGTTGGTGT TACTATATGG CAACCCAAAA AAGAGTCCAA GAAGTCAGAG GGTACACAAA GGGCGGTCCC |
| | CACCTTCTCC GCCAACCACA ATGATATACC GTTGGGTTTT TTCTCAGGTT CTTCAGTCTC CCATGTGTTT CCCGCCAGGG |
| | NS5 |
| | --------------------- |
| | G H E E P Q L • |
| | GGACATGAAG AGCCCCAACT |
| | CCTGTACTTC TCGGGGTTGA |
| | NS5 |
| | ---------------------------------------------------------------------------------------- |
| | • V Q S Y G W N I V T M K S G V D V F Y R P S E C C D T |
| 7701 | AGTGCAAAGT TATGGATGGA ACATTGTCAC CATGAAGAGT GGAGTGGATG TGTTCTACAG ACCTTCTGAG TGTTGTGACA |
| | TCACGTTTCA ATACCTACCT TGTAACAGTG GTACTTCTCA CCTCACCTAC ACAAGATGTC TGGAAGACTC ACAACACTGT |
| | NS5 |
| | --------------------- |
| | L L C D I G |
| | CCCTCCTTTG TGACATCGGA |
| | GGGAGGAAAC ACTGTAGCCT |
| | NS5 |
| | ---------------------------------------------------------------------------------------- |
| | E S S S S A E V E E H R T I R V L E M V E D W L H R G |
| 7801 | GAGTCCTCGT CAAGTGCTGA GGTTGAAGAG CATAGGACGA TTCGGGTCCT TGAAATGGTT GAGGACTGGC TGCACCGAGG |
| | CTCAGGAGCA GTTCACGACT CCAACTTCTC GTATCCTGCT AAGCCCAGGA ACTTTACCAA CTCCTGACCG ACGTGGCTCC |
| | NS5 |
| | --------------------- |
| | P R E F C V K • |
| | GCCAAGGGAA TTTTGCGTGA |
| | CGGTTCCCTT AAAACGCACT |
| | NS5 |
| | ---------------------------------------------------------------------------------------- |
| | • V L C P Y M P K V I E K M E L L Q R R Y G G G L V R |
| 7901 | AGGTGCTCTG CCCCTACATG CCGAAAGTCA TAGAGAAGAT GGAGCTGCTC CAACGCCGGT ATGGGGGGGG ACTGGTCAGA |
| | TCCACGAGAC GGGGATGTAC GGCTTTCAGT ATCTCTTCTA CCTCGACGAG GTTGCGGCCA TACCCCCCCC TGACCAGTCT |
| | NS5 |
| | --------------------- |
| | N P L S R N S • |
| | AACCCACTCT CACGGAATTC |
| | TTGGGTGAGA GTGCCTTAAG |
| | NS5 |
| | ---------------------------------------------------------------------------------------- |
| | • T H E M Y W V S R A S G N V V H S V N M T S Q V L L G |
| 8001 | CACGCACGAG ATGTATTGGG TGAGTCGAGC TTCAGGCAAT GTGGTACATT CAGTGAATAT GACCAGCCAG GTGCTCCTAG |
| | GTGCGTGCTC TACATAACCC ACTCAGCTCG AAGTCCGTTA CACCATGTAA GTCACTTATA CTGGTCGGTC CACGAGGATC |
| | NS5 |
| | --------------------- |
| | R M E K R T |
| | GAAGAATGGA AAAAAGGACC |
| | CTTCTTACCT TTTTTCCTGG |
| | NS5 |
| | ---------------------------------------------------------------------------------------- |
| | W K G P Q Y E E D V N L G S G T R A V G K P L L N S D |
| 8101 | TGGAAGGGAC CCCAATACGA GGAAGACGTA AACTTGGGAA GTGGAACCAG GGCGGTGGGA AAACCCCTGC TCAACTCAGA |
| | ACCTTCCCTG GGGTTATGCT CCTTCTGCAT TTGAACCCTT CACCTTGGTC CCGCCACCCT TTTGGGGACG AGTTGAGTCT |
| | NS5 |
| | --------------------- |
| | T S K I K N R • |
| | CACCAGTAAA ATCAAGAACA |
| | GTGGTCATTT TAGTTCTTGT |
| | NS5 |
| | ---------------------------------------------------------------------------------------- |
| | • I E R L R R E Y S S T W H H D E N H P Y R T W N Y H |
| 8201 | GGATTGAACG ACTCAGGCGT GAGTACAGTT CGACGTGGCA CCACGATGAG AACCACCCAT ATAGAACCTG GAACTATCAT |
| | CCTAACTTGC TGAGTCCGCA CTCATGTCAA GCTGCACCGT GGTGCTACTC TTGGTGGGTA TATCTTGGAC CTTGATAGTA |
| | NS5 |
| | --------------------- |
| | G S Y D V K P • |
| | GGCAGTTATG ATGTGAAGCC |
| | CCGTCAATAC TACACTTCGG |
| | NS5 |
| | ---------------------------------------------------------------------------------------- |
| | • T G S A S S L V N G V V R L L S K P W D T I T N V T T |
| 8301 | CACAGGCTCC GCCAGTTCGC TGGTCAATGG AGTGGTCAGG CTCCTCTCAA AACCATGGGA CACCATCACG AATGTTACCA |
| | GTGTCCGAGG CGGTCAAGCG ACCAGTTACC TCACCAGTCC GAGGAGAGTT TTGGTACCCT GTGGTAGTGC TTACAATGGT |
| | NS5 |
| | --------------------- |
| | M A M T D T |
| | CCATGGCCAT GACTGACACT |
| | GGTACCGGTA CTGACTGTGA |
| | NS5 |
| | ---------------------------------------------------------------------------------------- |
| | T P F G Q Q R V F K E K V D T K A P E P P E G V K Y V |
| 8401 | ACTCCCTTCG GGCAGCAGCG AGTGTTCAAA GAGAAGGTGG ACACGAAAGC TCCTGAACCG CCAGAAGGAG TGAAGTACGT |
| | TGAGGGAAGC CCGTCGTCGC TCACAAGTTT CTCTTCCACC TGTGCTTTCG AGGACTTGGC GGTCTTCCTC ACTTCATGCA |
| | NS5 |
| | --------------------- |
| | L N E T T N W • |
| | GCTCAACGAG ACCACCAACT |
| | CGAGTTGCTC TGGTGGTTGA |
| | NS5 |
| | ---------------------------------------------------------------------------------------- |
| | • L W A F L A R E K R P R M C S R E E F I R K V N S N |
| 8501 | GGTTGTGGGC GTTTTTGGCC AGAGAAAAAC GTCCCAGAAT GTGCTCTCGA GAGGAATTCA TAAGAAAGGT CAACAGCAAT |
| | CCAACACCCG CAAAAACCGG TCTCTTTTTG CAGGGTCTTA CACGAGAGCT CTCCTTAAGT ATTCTTTCCA GTTGTCGTTA |
| | NS5 |
| | --------------------- |
| | A A L G A M F • |
| | GCAGCTTTGG GTGCCATGTT |
| | CGTCGAAACC CACGGTACAA |
| | NS5 |
| | ---------------------------------------------------------------------------------------- |
| | • E E Q N Q W R S A R E A V E D P K F W E M V D E E R E |
| 8601 | TGAAGAGCAG AATCAATGGA GGAGCGCCAG AGAAGCAGTT GAAGATCCAA AATTTTGGGA AATGGTGGAT GAGGAGCGCG |
| | ACTTCTCGTC TTAGTTACCT CCTCGCGGTC TCTTCGTCAA CTTCTAGGTT TTAAAACCCT TTACCACCTA CTCCTCGCGC |
| | NS5 |
| | --------------------- |
| | A H L R G E |
| | AGGCACATCT GCGGGGGGAA |
| | TCCGTGTAGA CGCCCCCCTT |
| | NS5 |
| | ---------------------------------------------------------------------------------------- |
| | C H T C I Y N M M G K R E K K P G E F G K A K G S R A |
| 8701 | TGTCACACTT GCATTTACAA CATGATGGGA AACAGAGAGA AAAAACCCGG AGAGTTCGGA AAGGCCAAGG GAAGCAGAGC |
| | ACAGTGTGAA CGTAAATGTT GTACTACCCT TTCTCTCTCT TTTTTGGGCC TCTCAAGCCT TTCCGGTTCC CTTCGTCTCG |
| | NS5 |
| | --------------------- |
| | I W F M W L G • |
| | CATTTGGTTC ATGTGGCTCG |
| | GTAAACCAAG TACACCGAGC |
| | NS5 |
| | ---------------------------------------------------------------------------------------- |
| | • A R F L E F E A L G F L N E D H W L G R K N S G G G |
| 8801 | GAGCTCGCTT TCTGGAGTTC GAGGCTCTGG GTTTTCTCAA TGAAGACCAC TGGCTTGGAA GAAAGAACTC AGGAGGAGGT |
| | CTCGAGCGAA AGACCTCAAG CTCCGAGACC CAAAAGAGTT ACTTCTGGTG ACCGAACCTT CTTTCTTGAG TCCTCCTCCA |
| | NS5 |
| | --------------------- |
| | V E G L G L Q • |
| | GTCGAGGGCT TGGGCCTCCA |
| | CAGCTCCCGA ACCCGGAGGT |
| | NS5 |
| | ---------------------------------------------------------------------------------------- |
| | • K L G Y I L R E V G I R P G G K I Y A D D T A G W D T |
| 8901 | AAAACTGGGT TACATCCTGC GTGAAGTTGG CATCCGGCCT GGGGGCAAGA TCTATGCTGA TGACACAGCT GGCTGGGACA |
| | TTTTGACCCA ATGTAGGACG CACTTCAACC GTAGGCCGGA CCCCCGTTCT AGATACGACT ACTGTGTCGA CCGACCCTGT |
| | NS5 |
| | --------------------- |
| | R I T R A D |
| | CCCGCATCAC GAGAGCTGAC |
| | GGGCGTAGTG CTCTCGACTG |
| | NS5 |
| | ---------------------------------------------------------------------------------------- |
| | L E N E A K V L E L L D G E H R R L A R A I I E L T Y |
| 9001 | TTGGAAAATG AAGCTAAGGT GCTTGAGCTG CTTGATGGGG AACATCGGCG TCTTGCCAGG GCCATCATTG AGCTCACCTA |
| | AACCTTTTAC TTCGATTCCA CGAACTCGAC GAACTACCCC TTGTAGCCGC AGAACGGTCC CGGTAGTAAC TCGAGTGGAT |
| | NS5 |
| | --------------------- |
| | R H K V V K V • |
| | TCGTCACAAA GTTGTGAAAG |
| | AGGAGTGTTT CAACACTTTC |
| | NS5 |
| | ---------------------------------------------------------------------------------------- |
| | • M R P A A D G R T V M D V I S R E D Q R G S G Q V V |
| 9101 | TGATGCGCCC GGCTGCTGAT GGAAGAACCG TTATGGATGT TATCTCCAGA GAAGATCAGA GGGGGAGTGG ACAAGTTGTC |
| | ACTACGCGGG CCGACGACTA CCTTCTTGGC AATACCTACA ATAGAGGTCT CTTCTAGTCT CCCCCTCACC TGTTCAACAG |
| | NS5 |
| | --------------------- |
| | T Y A L N T F • |
| | ACCTACGCCC TAAACACTTT |
| | TGGATGCGGG ATTTGTGAAA |
| | NS5 |
| | ---------------------------------------------------------------------------------------- |
| | • T N L A V Q L V R M M E G E G V I G P D D V E K L T K |
| 9201 | CACCAACCTG GCTGTCCAGC TGGTGAGGAT GATGGAAGGG GAAGGAGTGA TTGGCCCAGA TGATGTGGAG AAACTCACAA |
| | GTGGTTGGAC CGACAGGTCG ACCACTCCTA CTACCTTCCC CTTCCTCACT AACCGGGTCT ACTACACCTC TTTGAGTGTT |
| | NS5 |
| | --------------------- |
| | G K G P K V |
| | AAGGGAAAGG ACCCAAAGTC |
| | TTCCCTTTCC TGGGTTTCAG |
| | NS5 |
| | ---------------------------------------------------------------------------------------- |
| | R T W L P E N G E E R L S R M A V S G D D C V V K P L |
| 9301 | AGGACCTGGC TGTTTGAGAA TGGGGAAGAA AGACTCAGCC GCATGGCTGT CAGTGGAGAT GACTGTGTGG TAAAGCCCCT |
| | TCCTGGACCG ACAAACTCTT ACCCCTTCTT TCTGAGTCGG CGTACCGACA GTCACCTCTA CTGACACACC ATTTCGGGGA |
| | NS5 |
| | --------------------- |
| | D D R F A T S • |
| | GGACGATCGC TTTGCCACCT |
| | CCTGCTAGCG AAACGGTGGA |
| | NS5 |
| | ---------------------------------------------------------------------------------------- |
| | • L H F L N A M S K V R K D I Q E W K P S T G W Y D W |
| 9401 | CGCTCCACTT CCTCAATGCT ATGTCAAAGG TTCGCAAAGA CATCCAAGAG TGGAAACCGT CAACTGGATG GTATGATTGG |
| | GCGAGGTGAA GGAGTTACGA TACAGTTTCC AAGCGTTTCT GTAGGTTCTC ACCTTTGGCA GTTGACCTAC CATACTAACC |
| | NS5 |
| | --------------------- |
| | Q Q V P F C S • |
| | CAGCAGGTTC CATTTTGCTC |
| | GTCGTCCAAG GTAAAACGAG |
| | NS5 |
| | ---------------------------------------------------------------------------------------- |
| | • N H F T E L I M K D G R T L V V P C R G Q D E L V G R |
| 9501 | AAACCATTTC ACTGAATTGA TCATGAAAGA TGGAAGAACA CTGGTGGTTC CATGCCGAGG ACAGGATGAA TTGGTAGGCA |
| | TTTGGTAAAG TGACTTAACT AGTACTTTCT ACCTTCTTGT GACCACCAAG GTACGGCTCC TGTCCTACTT AACCATCCGT |
| | NS5 |
| | --------------------- |
| | A R I S P G |
| | GAGCTCGCAT ATCTCCAGGG |
| | CTCGAGCGTA TAGAGGTCCC |
| | NS5 |
| | ---------------------------------------------------------------------------------------- |
| | A G W N V R D T A C L A K S Y A Q M W L L L Y F H R R |
| 9601 | GCCGGATGGA ACGTCCGCGA CACTGCTTGT CTGGCTAAGT CTTATGCCCA GATGTGGCTG CTTCTGTACT TCCACAGAAG |
| | CGGCCTACCT TGCAGGCGCT GTGACGAACA GACCGATTCA GAATACGGGT CTACACCGAC GAAGACATGA AGGTGTCTTC |
| | NS5 |
| | --------------------- |
| | D L R L M A N • |
| | AGACCTGCGG CTCATGGCCA |
| | TCTGGACGCC GAGTACCGGT |
| | NS5 |
| | ---------------------------------------------------------------------------------------- |
| | • A I C S A V P V N W V P T G R T T W S I H A G G E W |
| 9701 | ACGCCATTTG CTCCGCTGTC CCTGTGAATT GGGTCCCTAC CGGAAGAACC ACGTGGTCCA TCCATGCAGG AGGAGAGTGG |
| | TGCGGTAAAC GAGGCGACAG GGACACTTAA CCCAGGGATG GCCTTCTTGG TGCACCAGGT AGGTACGTCC TCCTCTCACC |
| | NS5 |
| | --------------------- |
| | M T T E D M L • |
| | ATGACAACAG AGGACATGTT |
| | TACTGTTGTC TCCTGTACAA |
| | NS5 |
| | ---------------------------------------------------------------------------------------- |
| | • E V W N R V W I E E N E W M E D K T P V E K W S D V P |
| 9801 | GGAGGTCTGG AACCGTGTTT GGATAGAGGA GAATGAATGG ATGGAAGACA AAACCCCAGT GGAGAAATGG AGTGACGTCC |
| | CCTCCAGACC TTGGCACAAA CCTATCTCCT CTTACTTACC TACCTTCTGT TTTGGGGTCA CCTCTTTACC TCACTGCAGG |
| | NS5 |
| | --------------------- |
| | Y S G K R E |
| | CATATTCAGG AAAACGAGAG |
| | GTATAAGTCC TTTTGCTCTC |
| | NS5 |
| | ---------------------------------------------------------------------------------------- |
| | D I W C G S L I G T R A R A T W A E N I Q V A I N Q V |
| 9901 | GACATCTGGT GTGGCAGCCT GATTGGCACA AGAGCCCGAG CCACGTGGGC AGAAAACATC CAGGTGGCTA TCAACCAAGT |
| | CTGTAGACCA CACCGTCGGA CTAACCGTGT TCTCGGGCTC GGTGCACCCG TCTTTTGTAG GTCCACCGAT AGTTGGTTCA |
| | NS5 |
| | --------------------- |
| | R A I I G D E • |
| | CAGAGCAATC ATCGGAGATG |
| | GTCTCGTTAG TAGCCTCTAC |
| | 3′ UTR |
| | ---------- |
| | NS5 |
| | ------------------------------------------------------------------------------ |
| | • K Y V D Y M S S L K R Y E D T T L V E D T V L |
| 10001 | AGAAGTATGT GGATTACATG AGTTCACTAA AGAGATATGA AGACACAACT TTGGTTGAGG ACACAGTACT GTAGATATTT |
| | TCTTCATACA CCTAATGTAC TCAAGTGATT TCTCTATACT TCTGTGTTGA AACCAACTCC TGTGTCATGA CATCTATAAA |
| | 3′ UTR |
| | --------------------- |
| | AATCAATTGT AAATAGACAA |
| | TTAGTTAACA TTTATCTGTT |
| | 3′ UTR |
| | ---------------------------------------------------------------------------------------- |
| 10101 | TATAAGTATG CATAAAAGTG TAGTTTTATA GTAGTATTTA GTGGTGTTAG TGTAAATAGT TAAGAAAATC TTGAGGAGAA |
| | ATATTCATAC GTATTTTCAC ATCAAAATAT CATCATAAAT CACCACAATC ACATTTATCA ATTCTTTTAG AACTCCTCTT |
| | 3′ UTR |
| | --------------------- |
| | AGTCAGGCCG GGAAGTTCCC |
| | TCAGTCCGGC CCTTCAAGGG |
| | 3′ UTR |
| | ---------------------------------------------------------------------------------------- |
| 10201 | GCCACCGGAA GTTGAGTAGA CGGTGCTGCC TGCGACTCAA CCCCAGGAGG ACTGGGTGAA CAAAGCCGCG AAGTGATCCA |
| | CGGTGGCCTT CAACTCATCT GCCACGACGG ACGCTGAGTT GGGGTCCTCC TGACCCACTT GTTTCGGCGC TTCACTAGGT |
| | 3′ UTR |
| | --------------------- |
| | TGTAAGCCCT CAGAACCGTC |
| | ACATTCGGGA GTCTTGGCAG |
| | 3′ UTR |
| | ---------------------------------------------------------------------------------------- |
| 10301 | TCGGAAGGAG GACCCCACAT GTTGTAACTT CAAAGCCCAA TGTCAGACCA CGCTACGGCG TGCTACTCTG CGGAGAGTGC |
| | AGCCTTCCTC CTGGGGTGTA CAACATTGAA GTTTCGGGTT ACAGTCTGGT GCGATGCCGC ACGATGAGAC GCCTCTCACG |
| | 3′ UTR |
| | --------------------- |
| | AGTCTGCGAT AGTGCCCCAG |
| | TCAGACGCTA TCACGGGGTC |
| | 3′ UTR |
| | ---------------------------------------------------------------------------------------- |
| 10401 | GAGGACTGGG TTAACAAAGG CAAACCAACG CCCCACGCGG CCCAAGCCCC GGTAATGGTG TTAACCAGGG CGAAAGGACT |
| | CTCCTGACCC AATTGTTTCC GTTTGGTTGC GGGGTGCGCC GGGTTCGGGG CCATTACCAC AATTGGTCCC GCTTTCCTGA |
| | 3′ UTR |
| | --------------------- |
| | AGAGGTTAGA GGAGACCCCG |
| | TCTCCAATCT CCTCTGGGGC |
| | 3′ UTR |
| | ---------------------------------------------------------------------------------------- |
| 10501 | CGGTTTAAAG TGCACGGCCC AGCCTGGCTG AAGCTGTAGG TCAGGGGAAG GACTAGAGGT TAGTGGAGAC CCCGTGCCAC |
| | GCCAAATTTC ACGTGCCGGG TCGGACCGAC TTCGACATCC AGTCCCCTTC CTGATCTCCA ATCACCTCTG GGGCACGGTG |
| | 3′ UTR |
| | --------------------- |
| | AAAACACCAC AACAAAACAG |
| | TTTTGTGGTG TTGTTTTGTC |
| | 3′ UTR |
| | ---------------------------------------------------------------------------------------- |
| 10601 | CAAATAGACA CCTGGGATAG ACTAGGAGAT CTTCTGCTCT GCACAACCAG CCACACGGCA CAGTGCGCCG ACAATGGTGG |
| | GTTTATCTGT GGACCCTATC TGATCCTCTA GAAGACGAGA CGTGTTGGTC GGTGTGCCGT GTCACGCGGC TGTTACCACC |
| | 3′ UTR |
| | --------------------- |
| | CTGGTGGTGC GAGAACACAG |
| | GACCACCACG CTCTTGTGTC |
| | 3′ UTR |
| | ----- |
| 10701 | GATCT |
| | CTAGA |

RepliVax WN - Anchorless F inserted in place of ΔC.
F insert starts at nucleotide position 229 bp and ends at 1806 bp.

| | |
|---|---|
| | 5′ UTR |
| | ---------------------------------------------------------------------------------------- |
| 1 | AGTAGTTCGC CTGTGTGAGC TGACAAACTT AGTAGTGTTT GTGAGGATTA ACAACAATTA ACACAGTGCG AGCTGTTTCT |
| | TCATCAAGCG GACACACTCG ACTGTTTGAA TCATCACAAA CACTCCTAAT TGTTGTTAAT TGTGTCACGC TCGACAAAGA |
| | 5′ UTR |
| | ----------------- |
| | N- |
| | terminus of C |
| | ---- |
| | M S • |
| | TAGCACGAAG ATCTCGATGT |
| | ATCGTGCTTC TAGAGCTACA |
| | N-terminus of C |
| | ---------------------------------------------------------------------------------------- |
| | • K K P G G P G K S R A V N M L K R G M P R V L S L I |
| 101 | CTAAGAAACC AGGAGGGCCC GGCAAGAGCC GGGCTCTCAA TATGCTAAAA CGCGGAATGC CCCGCGTGTT GTCCTTGATT |
| | GATTCTTTGG TCCTCCCGGG CCGTTCTCGG CCCGACAGTT ATACGATTTT GCGCCTTACG GGGCGCACAA CAGGAACTAA |
| | NS3 cleavage |
| | --------------- |
| | N-terminus of C |
| | ------ |
| | G L K Q K K R • |
| | GGACTTAAGC AAAAGAAGCG |
| | CCTGAATTCG TTTTCTTCGC |
| | NS3 cleavage Anchorless RSV F |
| | - ---------------------------------------------------------- |
| | partial C signal |
| | ----------------------------- |
| | • G G K T G I A V I M E L P I I K A N A I T T I L I A V |
| 201 | AGGGGGCAAG ACTGGTATAG CTGTGATCAT GGAACTGCCC ATCATCAAGG CCAACGCCAT CACCACCATC CTGATCGCCG |
| | TCCCCCGTTC TGACCATATC GACACTAGTA CCTTGACGGG TAGTAGTTCC GGTTGCGGTA GTGGTGGTAG GACTAGCGGC |
| | Anchorless RSV F |
| | --------------------- |
| | T F C F A S |
| | TGACCTTCTG CTTCGCCAGC |
| | ACTGGAAGAC GAAGCGGTCG |
| | Anchorless RSV F |
| | ---------------------------------------------------------------------------------------- |
| | S Q N I T E E F Y Q S T C S A V S K G Y L S A L R T G |
| 301 | AGCCAGAACA TCACCGACCA ATTCTACCAG AGCACCTGCA GCGCCGTGAG CAAGGGCTAC CTGAGCGCCC TGCGGACCGG |
| | TCGGTCTTGT AGTGGCTCCT TAAGATGGTC TCGTGGACGT CGCGGCACTC GTTCCCGATG GACTCGCGGG ACGCCTGGCC |
| | Anchorless RSV F |
| | --------------------- |
| | W Y T S V I T • |
| | CTGGTACACC AGCGTGATCA |
| | GACCATGTGG TCGCACTAGT |
| | Anchorless RSV F |
| | ---------------------------------------------------------------------------------------- |
| | • I E L S N I K E N K C N G T D A K V K L I K Q E L D |
| 401 | CCATCGAGCT GTCCAACATC AAAGAAAACA AGTGCAACGG CACCGACGCC AAGGTGAAAC TGATCAAGCA GGAACTGGAC |
| | GGTAGCTCGA CAGGTTGTAG TTTCTTTTGT TCACGTTGCC GTGGCTGCGG TTCCACTTTG ACTAGTTCGT CCTTGACCTG |
| | Anchorless RSV F |
| | --------------------- |
| | K Y K N A V T • |
| | AAGTACAAGA ACGCCGTGAC |
| | TTCATGTTCT TGCGGCACTG |
| | Anchorless RSV F |
| | ---------------------------------------------------------------------------------------- |
| | • E L Q L L M Q S T P A A N N R A R R E L P R F M N Y T |
| 501 | CGAGCTGCAG CTGCTGATGC AGAGCACCCC TGCCGCCAAC AACCGGGCCA GACGCGAGCT GCCCCGGTTC ATGAACTACA |
| | GCTCGACGTC GACGACTACG TCTCGTGGGG ACGGCGGTTG TTGGCCCGGT CTGCGCTCGA CGGGGCCAAG TACTTGATGT |
| | Anchorless RSV F |
| | --------------------- |
| | L N N A K K |
| | CCCTGAACAA CGCCAAGAAA |
| | GGGACTTGTT GCGGTTCTTT |
| | Anchorless RSV F |
| | ---------------------------------------------------------------------------------------- |
| | T N V T L S K K R K R R F L G F L L G V G S A I A S G |
| 601 | ACCAACGTGA CCCTGAGCAA GAAGCGGAAG CGGCGGTTCC TGGGCTTCCT GCTGGGCGTG GGCAGCGCCA TCGCCAGCGG |
| | TGGTTGCACT GGGACTCGTT CTTCGCCTTC GCCGCCAAGG ACCCGAAGGA CGACCCGCAC CCGTCGCGGT AGCGGTCGCC |
| | Anchorless RSV F |
| | --------------------- |
| | I A V S K V L • |
| | CATCGCCGTG TCCAAGGTGC |
| | GTAGCGGCAC AGGTTCCACG |
| | Anchorless RSV F |
| | ---------------------------------------------------------------------------------------- |
| | • H L E G E V N K I K S A L L S T N K A V V S L S N G |
| 701 | TGCACCTGGA AGGCGAGGTG AACAAGATCA AGTCCGCCCT GCTGTCCACC AACAAGGCCG TGGTGTCCCT GAGCAACGGC |
| | ACGTGGACCT TCCGCTCCAC TTGTTCTAGT TCAGGCGGGA CGACAGGTGG TTGTTCCGGC ACCACAGGGA CTCGTTGCCG |
| | Anchorless RSV F |
| | --------------------- |
| | V S V L T S K • |
| | GTGAGCGTGC TGACCAGCAA |
| | CACTCGCACG ACTGGTCGTT |
| | Anchorless RSV F |
| | ---------------------------------------------------------------------------------------- |
| | • V L D L K N Y I D K Q L L P I V N K Q S C S I S N I E |
| 801 | GGTGCTGGAT CTGAAGAACT ACATCGACAA GCAGCTGCTG CCCATCGTGA ACAAGCAGAG CTGCAGCATC AGCAACATCG |
| | CCACGACCTA GACTTCTTGA TGTAGCTGTT CGTCGACGAC GGGTAGCACT TGTTCGTCTC GACGTCGTAG TCGTTGTAGC |
| | Anchorless RSV F |
| | --------------------- |
| | T V I E F Q |
| | AGACCGTGAT CGAGTTCCAG |
| | TCTGGCACTA GCTCAAGGTC |
| | Anchorless RSV F |
| | ---------------------------------------------------------------------------------------- |
| | Q K N N R L L E I T R E F S V N A G V T T P V S T Y M |
| 901 | CAGAAGAACA ACCGGCTGCT GGAAATCACC CGGGAGTTCA GCGTGAACGC CGGCGTGACC ACCCCCGTGA GCACCTACAT |
| | GTCTTCTTGT TGGCCGACGA CCTTTAGTGG GCCCTCAAGT CGCACTTGCG GCCGCACTGG TGGGGGCACT CGTGGATGTA |
| | Anchorless RSV F |
| | --------------------- |
| | L T N S E L L • |
| | GCTGACCAAC AGCGAGCTGC |
| | CGACTGGTTG TCGCTCGACG |
| | Anchorless RSV F |
| | ---------------------------------------------------------------------------------------- |
| | • S L I N D M P I T N D Q K K L M S N N V Q I V R Q Q |
| 1001 | TGTCCCTGAT CAATGACATG CCCATCACCA ACGACCAGAA GAAACTGATG AGCAACAACG TGCAGATCGT GCGGCAGCAG |
| | ACAGGGACTA GTTACTGTAC GGGTAGTGGT TGCTGGTCTT CTTTGACTAC TCGTTGTTGC ACGTCTAGCA CGCCGTCGTC |
| | Anchorless RSV F |
| | --------------------- |
| | S Y S I M S I • |
| | AGCTACTCCA TCATGAGCAT |
| | TCGATGAGGT AGTACTCGTA |
| | Anchorless RSV F |
| | ---------------------------------------------------------------------------------------- |
| | • I K E E V L A Y V V Q L P L Y G V I D T P C W K L H T |
| 1101 | CATCAAAGAA GAGGTGCTGG CCTACGTGGT GCAGCTGCCC CTGTACGGCG TGATCGACAC CCCCTGCTGG AAGCTGCACA |
| | GTAGTTTCTT CTCCACGACC GGATGCACCA CGTCGACGGG GACATGCCGC ACTAGCTGTG GGGGACGACC TTCGACGTGT |
| | Anchorless RSV F |
| | --------------------- |
| | S P L C T T |
| | CCAGCCCCCT GTGCACCACC |
| | GGTCGGGGGA CACGTGGTGG |
| | Anchorless RSV F |
| | ---------------------------------------------------------------------------------------- |
| | N T K E G S N I C L T R T D R G W Y C N N A G S V S F |
| 1201 | AACACCAAAG AGGGCAGCAA CATCTGCCTG ACCCGGACCG ACCGGGGCTG GTACTGCAAC AACGCCGGCA GCGTGAGCTT |
| | TTGTGGTTTC TCCCGTCGTT GTAGACGGAC TGGGCCTGGC TGGCCCCGAC CATGACGTTG TTGCGGCCGT CGCACTCGAA |
| | Anchorless RSV F |
| | --------------------- |
| | F P L A D T C • |
| | CTTCCCCCTG GCCGACACCT |
| | GAAGGGGGAC CGGCTGTGGA |
| | Anchorless RSV F |
| | ---------------------------------------------------------------------------------------- |
| | • K V Q S N R V F C D T M N S L T L P S E V N L C N I |
| 1301 | GCAAGGTGCA GAGCAACCGG GTGTTCTGCG ACACCATGAA CAGCCTGACC CTGCCCTCCG AGGTGAACCT GTGCAACATC |
| | CGTTCCACGT CTCGTTGGCC CACAAGACGC TGTGGTACTT GTCGGACTGG GACGGGAGGC TCCACTTGGA CACGTTGTAG |
| | Anchorless RSV F |
| | --------------------- |
| | D I F N P K Y • |
| | GACATCTTCA ACCCCAAGTA |
| | CTGTAGAAGT TGGGGTTCAT |
| | Anchorless RSV F |
| | ---------------------------------------------------------------------------------------- |
| | • D C K I M T S K T D V S S S V I T S L G A I V S C Y G |
| 1401 | CGACTGCAAG ATCATGACCT CCAAGACCGA CGTGAGCAGC TCCGTGATCA CCTCCCTGGG CGCCATCGTG AGCTGCTACG |
| | GCTGACGTTC TAGTACTGGA GGTTCTGGCT GCACTCGTCG AGGCACTAGT GGAGGGACCC GCGGTAGCAC TCGACGATGC |
| | Anchorless RSV F |
| | --------------------- |
| | K T K C T A |
| | GCAAGACCAA GTGCACCGCC |
| | CGTTCTGGTT CACGTGGCGG |
| | Anchorless RSV F |
| | ---------------------------------------------------------------------------------------- |
| | S N K N R G I I K T F S N G C D Y V S N K G V D T V S |
| 1501 | AGCAACAAGA ACCGGGGCAT CATCAAGACC TTCAGCAACG GCTGCGACTA CGTGAGCAAC AAGGGCGTGG ACACCGTGAG |
| | TCGTTGTTCT TGGCCCCGTA GTAGTTCTGG AAGTCGTTGC CGACGCTGAT GCACTCGTTG TTCCCGCACC TGTGGCACTC |
| | Anchorless RSV F |
| | --------------------- |
| | V G N T L Y Y • |
| | CGTGGGCAAC ACACTGTACT |
| | GCACCCGTTG TGTGACATGA |
| | Anchorless RSV F |
| | ---------------------------------------------------------------------------------------- |
| | • V N K Q E G K S L Y V K G E P I I N F Y D P L V F P |
| 1601 | ACGTGAATAA GCAGGAAGGC AAGAGCCTGT ACGTGAAGGG CGAGCCTATC ATCAACTTCT ACGACCCCCT GGTGTTCCCC |
| | TGCACTTATT CGTCCTTCCG TTCTCGGACA TGCACTTCCC GCTCGGATAG TAGTTGAAGA TGCTGGGGGA CCACAAGGGG |
| | Anchorless RSV F |
| | --------------------- |
| | S D E F D A S • |
| | AGCGACGAGT TCGACGCCAG |
| | TCGCTGCTCA AGCTGCGGTC |
| | Anchorless RSV F |
| | ---------------------------------------------------------------------------------------- |
| | • I S Q V N E K I N Q S L A F I R K S D E L L H N V N A |
| 1701 | CATCAGCCAG GTGAACGAGA AGATCAACCA GAGCCTGGCC TTCATCCGGA AGAGCGACGA GCTGCTGCAC AATGTGAATG |
| | GTAGTCGGTC CACTTGCTCT TCTAGTTGGT CTCGGACCGG AAGTAGGCCT TCTCGCTGCT CGACGACGTG TTACACTTAC |
| | Anchorless RSV F |
| | --------------------- |
| | G K S T T N |
| | CCGGCAAGAG CACCACCAAT |
| | GGCCGTTCTC GTGGTGGTTA |
| | FMDV 2A |
| | -------------------------------------------------------- |
| | Anchorless RSV F C/prM signal |
| | ------ ------------------------- |
| | I M N F D L L K L A G D V E S N P G P G G K T G I A V |
| 1801 | ATCATGAATT TTGATCTGCT CAAACTTGCA GGCGATGTAG AATCAAATCC TGGACCCGGA GGAAAGACCG GTATTGCAGT |
| | TAGTACTTAA AACTAGACGA GTTTGAACGT CCGCTACATC TTAGTTTAGG ACCTGGGCCT CCTTTCTGGC CATAACGTCA |
| | C/prM signal |
| | --------------------- |
| | M I G L I A C • |
| | CATGATTGGC CTGATCGCCT |
| | GTACTAACCG GACTAGCGGA |
| | prM |
| | ---------------------------------------------------------------------------- |
| | C/prM signal |
| | ------------ |
| | • V G A V T L S N F Q G K V M M T V N A T D V T D V I |
| 1901 | GCGTAGGAGC AGTTACCCTC TCTAACTTCC AAGGGAAGGT GATGATGACG GTAAATGCTA CTGACGTCAC AGATGTCATC |
| | CGCATCCTCG TCAATGGGAG AGATTGAAGG TTCCCTTCCA CTACTACTGC CATTTACGAT GACTGCAGTG TCTACAGTAG |
| | prM |
| | --------------------- |
| | T I P T A A G • |
| | ACGATTCCAA CAGCTGCTGG |
| | TGCTAAGGTT GTCGACGACC |
| | prM |
| | ---------------------------------------------------------------------------------------- |
| | • K N L C I V R A M D V G Y M C D D T I T Y E C P V L S |
| 2001 | AAAGAACCTA TGCATTGTCA GAGCAATGGA TGTGGGATAC ATGTGCGATG ATACTATCAC TTATGAATGC CCAGTGCTGT |
| | TTTCTTGGAT ACGTAACAGT CTCGTTACCT ACACCCTATG TACACGCTAC TATGATAGTG AATACTTACG GGTCACGACA |
| | prM |
| | --------------------- |
| | A G N D P E |
| | CGGCTGGTAA TGATCCAGAA |
| | GCCGACCATT ACTAGGTCTT |
| | prM |
| | ---------------------------------------------------------------------------------------- |
| | D I D C W C T K S A V Y V R Y G R C T K T R H S R R S |
| 2101 | GACATCGACT GTTGGTGCAC AAAGTCAGCA GTCTACGTCA GGTATGGAAG ATGCACCAAG ACACGCCACT CAAGACGCAG |
| | CTGTAGCTGA CAACCACGTG TTTCAGTCGT CAGATGCAGT CCATACCTTC TACGTGGTTC TGTGCGGTGA GTTCTGCGTC |
| | prM |
| | --------------------- |
| | R R S L T V Q • |
| | TCGGAGGTCA CTGACAGTGC |
| | AGCCTCCAGT GACTGTCACG |
| | prM |
| | ---------------------------------------------------------------------------------------- |
| | • T H G E S T L A N K K G A W M D S T K A T R Y L V K |
| 2201 | AGACACACGG AGAAAGCACT CTAGCGAACA AGAAGGGGGC TTGGATGGAC AGCACCAAGG CCACAAGGTA TTTGGTAAAA |
| | TCTGTGTGCC TCTTTCGTGA GATCGCTTGT TCTTCCCCCG AACCTACCTG TCGTGGTTCC GGTGTTCCAT AAACCATTTT |
| | prM |
| | --------------------- |
| | T E S W I L R • |
| | ACAGAATCAT GGATCTTGAG |
| | TGTCTTAGTA CCTAGAACTC |
| | prM |
| | ---------------------------------------------------------------------------------------- |
| | • N P G Y A L V A A V I G W M L G S N T M Q R V V F V V |
| 2301 | GAACCCTGGA TATGCCCTGG TGGCAGCCGT CATTGGTTGG ATGCTTGGGA GCAACACCAT GCAGAGAGTT GTGTTTGTCG |
| | CTTGGGACCT ATACGGGACC ACCGTCGGCA GTAACCAACC TACGAACCCT CGTTGTGGTA CGTCTCTCAA CACAAACAGC |
| | prM |
| | --------------------- |
| | L L L L V A |
| | TGCTATTGCT TTTGGTGGCC |
| | ACGATAACGA AAACCACCGG |
| | prM |
| | ------------- |
| | E |
| | --------------------------------------------------------------------------- |
| | P A Y S F N C L G M S N R D F L E G V S G A T W V D L |
| 2401 | CCAGCTTACA GCTTTAACTG CCTTGGAATG AGCAACAGAG ACTTCTTGGA AGGAGTGTCT GGAGCAACAT GGGTGGATTT |
| | GGTCGAATGT CGAAATTGAC GGAACCTTAC TCGTTGTCTC TGAAGAACCT TCCTCACAGA CCTCGTTGTA CCCACCTAAA |
| | E |
| | --------------------- |
| | V L E G D S C • |
| | GGTTCTCGAA GGCGACAGCT |
| | CCAAGAGCTT CCGCTGTCGA |
| | E |
| | ---------------------------------------------------------------------------------------- |
| | • V T I M S K D K P T I D V K M M N M E A A N L A E V |
| 2501 | GCGTGACTAT CATGTCTAAG GACAAGCCTA CCATCGATGT GAAGATGATG AATATGGAGG CGGCCAACCT GGCAGAGGTC |
| | CGCACTGATA GTACAGATTC CTGTTCGGAT GGTAGCTACA CTTCTACTAC TTATACCTCC GCCGGTTGGA CCGTCTCCAG |
| | E |
| | --------------------- |
| | R S Y C Y L A • |
| | CGCAGTTATT GCTATTTGGC |
| | GCGTCAATAA CGATAAACCG |
| | E |
| | ---------------------------------------------------------------------------------------- |
| | • T V S D L S T K A A C P A M G E A H N D K R A D P A F |
| 2601 | TACCGTCAGC GATCTCTCCA CCAAAGCTGC GTGCCCGGCC ATGGGAGAAG CTCACAATGA CAAACGTGCT GACCCAGCTT |
| | ATGGCAGTCG CTAGAGAGGT GGTTTCGACG CACGGGCCGG TACCCTCTTC GAGTGTTACT GTTTGCACGA CTGGGTCGAA |
| | E |
| | --------------------- |
| | V C R Q G V |
| | TTGTGTGCAG ACAAGGAGTG |
| | AACACACGTC TGTTCCTCAC |
| | E |
| | ---------------------------------------------------------------------------------------- |
| | V D R G W G N G C G L F G K G S I D T C A K F A C S T |
| 2701 | GTGGACAGGG GCTGGGGCAA CGGCTGCGGA CTATTTGGCA AAGGAAGCAT TGACACATGC GCCAAATTTG CCTGCTCTAC |
| | CACCTGTCCC CGACCCCGTT GCCGACGCCT GATAAACCGT TTCCTTCGTA ACTGTGTACG CGGTTTAAAC GGACGAGATG |
| | E |
| | --------------------- |
| | K A I G R T I • |
| | CAAGGCAATA GGAAGAACCA |
| | GTTCCGTTAT CCTTCTTGGT |
| | E |
| | ---------------------------------------------------------------------------------------- |
| | • L K E N I K Y E V A I F V H G P T T V E S H G N Y S |
| 2801 | TTTTGAAAGA GAATATCAAG TACGAAGTGG CCATTTTTGT CCATGGACCA ACTACTGTGG AGTCGCACGG AAACTACTCC |
| | AAAACTTTCT CTTATAGTTC ATGCTTCACC GGTAAAAACA GGTACCTGGT TGATGACACC TCAGCGTGCC TTTGATGAGG |
| | E |
| | --------------------- |
| | T Q V G A T Q • |
| | ACACAGGTTG GAGCCACTCA |
| | TGTGTCCAAC CTCGGTGAGT |
| | E |
| | ---------------------------------------------------------------------------------------- |
| | • A G R F S I T P A A P S Y T L K L G E Y G E V T V D C |
| 2901 | GGCAGGGAGA TTCAGCATCA CTCCTGCGGC GCCTTCATAC ACACTAAAGC TTGGAGAATA TGGAGAGGTG ACAGTGGACT |
| | CCGTCCCTCT AAGTCGTAGT GAGGACGCCG CGGAAGTATG TGTGATTTCG AACCTCTTAT ACCTCTCCAC TGTCACCTGA |
| | E |
| | --------------------- |
| | E P R S G I |
| | GTGAACCACG GTCAGGGATT |
| | CACTTGGTGC CAGTCCCTAA |
| | E |
| | ---------------------------------------------------------------------------------------- |
| | D T N A Y Y V M T V G T K T F L V H R E W F M D L N L |
| 3001 | GACACCAATG CATACTACGT GATGACTGTT GGAACAAAGA CGTTCTTGGT CCATCGTGAG TGGTTCATGG ACCTCAACCT |
| | CTGTGGTTAC GTATGATGCA CTACTGACAA CCTTGTTTCT GCAAGAACCA GGTAGCACTC ACCAAGTACC TGGAGTTGGA |
| | E |
| | --------------------- |
| | P W S S A G S • |
| | CCCTTGGAGC AGTGCTGGAA |
| | GGGAACCTCG TCACGACCTT |
| | E |
| | ---------------------------------------------------------------------------------------- |
| | • T V W R N R E T L M E F E E P H A T K Q S V I A L G |
| 3101 | GTACTGTGTG GAGGAACAGA GAGACGTTAA TGGAGTTTGA GGAACCACAC GCCACGAAGC AGTCTGTGAT AGCATTGGGC |
| | CATGACACAC CTCCTTGTCT CTCTGCAATT ACCTCAAACT CCTTGGTGTG CGGTGCTTCG TCAGACACTA TCGTAACCCG |
| | E |
| | --------------------- |
| | S Q E G A L H • |
| | TCACAAGAGG GAGCTCTGCA |
| | AGTGTTCTCC CTCGAGACGT |
| | E |
| | ---------------------------------------------------------------------------------------- |
| | • Q A L A G A I P V E F S S N T V K L T S G H L K C R V |
| 3201 | TCAAGCTTTG GCTGGAGCCA TTCCTGTGGA ATTTTCAAGC AACACTGTCA AGTTGACGTC GGGTCATTTG AAGTGTAGAG |
| | AGTTCGAAAC CGACCTCGGT AAGGACACCT TAAAAGTTCG TTGTGACAGT TCAACTGCAG CCCAGTAAAC TTCACATCTC |
| | E |
| | --------------------- |
| | K M E K L Q |
| | TGAAGATGGA AAAATTGCAG |
| | ACTTCTACCT TTTTAACGTC |
| | E |
| | ---------------------------------------------------------------------------------------- |
| | L K G T T Y G V C S K A F K F L G T P A D T G H G T V |
| 3301 | TTGAAGGGAA CAACCTATGG CGTCTGTTCA AAGGCTTTCA AGTTTCTTGG GACTCCCGCA GACACAGGTC ACGGCACTGT |
| | AACTTCCCTT GTTGGATACC GCAGACAAGT TTCCGAAAGT TCAAAGAACC CTGAGGGCGT CTGTGTCCAG TGCCGTGACA |
| | E |
| | --------------------- |
| | V L E L Q Y T • |
| | GGTGTTGGAA TTGCAGTACA |
| | CCACAACCTT AACGTCATGT |
| | E |
| | ---------------------------------------------------------------------------------------- |
| | • G T D G P C K V P I S S V A S L N D L T P V G R L V |
| 3401 | CTGGCACGGA TGGACCTTGC AAAGTTCCTA TCTCGTCAGT GGCTTCATTG AACGACCTAA CGCCAGTGGG CAGATTGGTC |
| | GACCGTGCCT ACCTGGAACG TTTCAAGGAT AGAGCAGTCA CCGAAGTAAC TTGCTGGATT GCGGTCACCC GTCTAACCAG |
| | E |
| | --------------------- |
| | T V N P F V S • |
| | ACTGTCAACC CTTTTGTTTC |
| | TGACAGTTGG GAAAACAAAG |
| | E |
| | ---------------------------------------------------------------------------------------- |
| | • V A T A N A K V L I E L E P P F G D S Y I V V G R G E |
| 3501 | AGTGGCCACG GCCAACGCTA AGGTCCTGAT TGAATTGGAA CCACCCTTTG GAGACTCATA CATAGTGGTG GGCAGAGGAG |
| | TCACCGGTGC CGGTTGCGAT TCCAGGACTA ACTTAACCTT GGTGGGAAAC CTCTGAGTAT GTATCACCAC CCGTCTCCTC |
| | E |
| | --------------------- |
| | Q Q I N H H |
| | AACAACAGAT CAATCACCAC |
| | TTGTTGTCTA GTTAGTGGTG |
| | E |
| | ---------------------------------------------------------------------------------------- |
| | W H K S G S S I G K A F T T T L K G A Q R L A A L G D |
| 3601 | TGGCACAAGT CTGGAAGCAG CATTGGCAAA GCCTTTACAA CCACCCTCAA AGGAGCGCAG AGACTAGCCG CTCTAGGAGA |
| | ACCGTGTTCA GACCTTCGTC GTAACCGTTT CGGAAATGTT GGTGGGAGTT TCCTCGCGTC TCTGATCGGC GAGATCCTCT |
| | E |
| | --------------------- |
| | T A W D F G S • |
| | CACAGCTTGG GACTTTGGAT |
| | GTGTCGAACC CTGAAACCTA |
| | E |
| | ---------------------------------------------------------------------------------------- |
| | • V G G V F T S V G K A V H Q V F G G A F R S L F G G |
| 3701 | CAGTTGGAGG GGTGTTCACC TCAGTTGGGA AGGCTGTCCA TCAAGTGTTC GGAGGAGCAT TCCGCTCACT GTTCGGAGGC |
| | GTCAACCTCC CCACAAGTGG AGTCAACCCT TCCGACAGGT AGTTCACAAG CCTCCTCGTA AGGCGAGTGA CAAGCCTCCG |
| | E |
| | --------------------- |
| | M S W I T Q G • |
| | ATGTCCTGGA TAACGCAAGG |
| | TACAGGACCT ATTGCGTTCC |
| | E |
| | ---------------------------------------------------------------------------------------- |
| | • L L G A L L L W M G I N A R D R S I A L T F L A V G G |
| 3801 | ATTGCTGGGG GCTCTCCTGT TGTGGATGGG CATCAATGCT CGTGACAGGT CCATAGCTCT CACGTTTCTC GCAGTTGGAG |
| | TAACGACCCC CGAGAGGACA ACACCTACCC GTAGTTACGA GCACTGTCCA GGTATCGAGA GTGCAAAGAG CGTCAACCTC |
| | E |
| | --------------------- |
| | V L L F L S |
| | GAGTTCTGCT CTTCCTCTCC |
| | CTCAAGACGA GAAGGAGAGG |
| | NS1 |
| | ------------------------------------------------------------------------ |
| | E |
| | ---------------- |
| | V N V H A D T G C A I D I S R Q E L R C G S G V F I H |
| 3901 | GTGAACGTGC ACGCTGACAC TGGGTGTGCC ATAGACATCA GCCGGCAAGA GCTGAGATGT GGAAGTGGAG TGTTCATACA |
| | CACTTGCACG TGCGACTGTG ACCCACACGG TATCTGTAGT CGGCCGTTCT CGACTCTACA CCTTCACCTC ACAAGTATGT |
| | NS1 |
| | --------------------- |
| | N D V E A W M • |
| | CAATGATGTG GAGGCTTGGA |
| | GTTACTACAC CTCCGAACCT |
| | NS1 |
| | ---------------------------------------------------------------------------------------- |
| | • D R Y K Y Y P E T P Q G L A K I I Q K A H K E G V C |
| 4001 | TGGACCGGTA CAAGTATTAC CCTGAAACGC CACAAGGCCT AGCCAAGATC ATTCAGAAAG CTCATAAGGA AGGAGTGTGC |
| | ACCTGGCCAT GTTCATAATG GGACTTTGCG GTGTTCCGGA TCGGTTCTAG TAAGTCTTTC GAGTATTCCT TCCTCACACG |
| | NS1 |
| | --------------------- |
| | G L R S V S R • |
| | GGTCTACGAT CAGTTTCCAG |
| | CCAGATGCTA GTCAAAGGTC |
| | NS1 |
| | ---------------------------------------------------------------------------------------- |
| | • L E H Q M W E A V K D E L N T L L K E N G V D L S V V |
| 4101 | ACTGGAGCAT CAAATGTGGG AAGCAGTGAA GGACGAGCTG AACACTCTTT TGAAGGAGAA TGGTGTGGAC CTTAGTGTCG |
| | TGACCTCGTA GTTTACACCC TTCGTCACTT CCTGCTCGAC TTGTGAGAAA ACTTCCTCTT ACCACACCTG GAATCACAGC |
| | NS1 |
| | --------------------- |
| | V E K Q G G |
| | TGGTTGAGAA ACAAGGGGGA |
| | ACCAACTCTT TGTTCCCCCT |
| | NS1 |
| | ---------------------------------------------------------------------------------------- |
| | M Y K S A P K R L T A T T E K L E I G W K A W G K S I |
| 4201 | ATGTACAAGT CAGCACCTAA ACGCCTCACC GCCACCACGG AAAAATTGGA AATTGGCTGG AAGGCCTGGG GAAAGAGTAT |
| | TACATGTTCA GTCGTGGATT TGCGGAGTGG CGGTGGTGCC TTTTTAACCT TTAACCGACC TTCCGGACCC CTTTCTCATA |
| | NS1 |
| | --------------------- |
| | L F A P E L A • |
| | TTTGTTTGCA CCAGAACTCG |
| | AAACAAACGT GGTCTTGAGC |
| | NS1 |
| | ---------------------------------------------------------------------------------------- |
| | • N N T F V V D G P E T K E C P T Q N R A W N S L E V |
| 4301 | CCAACAACAC CTTTGTGGTT GATGGTCCGG AGACCAAGGA ATGTCCGACT CAGAATCGCG CTTGGAATAG CTTAGAAGTG |
| | GGTTGTTGTG GAAACACCAA CTACCAGGCC TCTGGTTCCT TACAGGCTGA GTCTTAGCGC GAACCTTATC GAATCTTCAC |
| | NS1 |
| | --------------------- |
| | E D F G F G L • |
| | GAGGATTTTG GATTTGGTCT |
| | CTCCTAAAAC CTAAACCAGA |
| | NS1 |
| | ---------------------------------------------------------------------------------------- |
| | • T S T R M F L K V R E S N T T E C D S K I I G T A V K |
| 4401 | CACCAGCACT CGGATGTTCC TGAAGGTCAG AGAGAGCAAC ACAACTGAAT GTGACTCGAA GATCATTGGA ACGGCTGTCA |
| | GTGGTCGTGA GCCTACAAGG ACTTCCAGTC TCTCTCGTTG TGTTGACTTA CACTGAGCTT CTAGTAACCT TGCCGACAGT |
| | NS1 |
| | --------------------- |
| | N N L A I H |
| | AGAACAACTT GGCGATCCAC |
| | TCTTGTTGAA CCGCTAGGTG |
| | NS1 |
| | ---------------------------------------------------------------------------------------- |
| | S D L S Y W I E S R L N D T W K L E R A V L G E V K S |
| 4501 | AGTGACCTGT CCTATTGGAT TGAAAGCAGG CTCAATGATA CGTGGAAGCT TGAAAGGGCA GTTCTGGGTG AAGTCAAATC |
| | TCACTGGACA GGATAACCTA ACTTTCGTCC GAGTTACTAT GCACCTTCGA ACTTTCCCGT CAAGACCCAC TTCAGTTTAG |
| | NS1 |
| | --------------------- |
| | C T W P E T H • |
| | ATGTACGTGG CCTGAGACGC |
| | TACATGCACC GGACTCTGCG |
| | NS1 |
| | ---------------------------------------------------------------------------------------- |
| | • T L W G D G I L E S D L I I P V T L A G P R S N H N |
| 4601 | ATACCTTGTG GGGCGATGGA ATCCTTGAGA GTGACTTGAT AATACCAGTC ACACTGGCGG GACCACGAAG CAATCACAAT |
| | TATGGAACAC CCCGCTACCT TAGGAACTCT CACTGAACTA TTATGGTCAG TGTGACCGCC CTGGTGCTTC GTTAGTGTTA |
| | NS1 |
| | --------------------- |
| | R R P G Y K T • |
| | CGGAGACCTG GGTATAAGAC |
| | GCCTCTGGAC CCATATTCTG |
| | NS1 |
| | ---------------------------------------------------------------------------------------- |
| | • Q N Q G P W D E G R V E I D F D Y C P G T T V T L S E |
| 4701 | ACAAAACCAG GGCCCATGGG ACGAAGGCCG GGTAGAGATT GACTTCGATT ACTGCCCAGG AACTACGGTC ACCCTGAGTG |
| | TGTTTTGGTC CCGGGTACCC TGCTTCCGGC CCATCTCTAA CTGAAGCTAA TGACGGGTCC TTGATGCCAG TGGGACTCAC |
| | NS1 |
| | --------------------- |
| | S C G H R G |
| | AGAGCTGCGG ACACCGTGGA |
| | TCTCGACGCC TGTGGCACCT |
| | NS1 |
| | ---------------------------------------------------------------------------------------- |
| | P A T R T T T E S G K L I T D W C C R S C T L P P L R |
| 4801 | CCTGCCACTC GCACCACCAC AGAGAGCGGA AAGTTGATAA CAGATTGGTG CTGCAGGAGC TGCACCTTAC CACCACTGCG |
| | GGACGGTGAG CGTGGTGGTG TCTCTCGCCT TTCAACTATT GTCTAACCAC GACGTCCTCG ACGTGGAATG GTGGTGACGC |
| | NS1 |
| | --------------------- |
| | Y Q T D S G C • |
| | CTACCAAACT GACAGCGGCT |
| | GATGGTTTGA CTGTCGCCGA |
| | NS2A |
| | --------- |
| | NS1 |
| | ------------------------------------------------------------------------------ |
| | • W Y G M E I R P Q R H D E K T L V Q S Q V N A Y N A |
| 4901 | GTTGGTATGG TATGGAGATC AGACCACAGA GACATGATGA AAAGACCCTC GTGCAGTCAC AAGTGAATGC TTATAATGCT |
| | CAACCATACC ATACCTCTAG TCTGGTGTCT CTGTACTACT TTTCTGGGAG CACGTCAGTG TTCACTTACG AATATTACGA |
| | NS2A |
| | --------------------- |
| | D M I D P F Q • |
| | GATATGATTG ACCCTTTTCA |
| | CTATACTAAC TGGGAAAAGT |
| | NS2A |
| | ---------------------------------------------------------------------------------------- |
| | • L G L L V V F L A T Q E V L R K R W T A K I S M P A I |
| 5001 | GTTGGGCCTT CTGGTCGTGT TCTTGGCCAC CCAGGAGGTC CTTCGCAAGA GGTGGACAGC CAAGATCAGC ATGCCAGCTA |
| | CAACCCGGAA GACCAGCACA AGAACCGGTG GGTCCTCCAG GAAGCGTTCT CCACCTGTCG GTTCTAGTCG TACGGTCGAT |
| | NS2A |
| | --------------------- |
| | L I A L L V |
| | TACTGATTGC TCTGCTAGTC |
| | ATGACTAACG AGACGATCAG |
| | NS2A |
| | ---------------------------------------------------------------------------------------- |
| | L V F G G I T Y T D V L R Y V I L V G A A F A E S N S |
| 5101 | CTGGTGTTTG GGGGCATTAC TTACACTGAT GTGTTACGCT ATGTCATCTT GGTGGGGGCA GCTTTCGCAG AATCTAATTC |
| | GACCACAAAC CCCCGTAATG AATGTGACTA CACAATGCGA TACAGTAGAA CCACCCCCGT CGAAAGCGTC TTAGATTAAG |
| | NS2A |
| | --------------------- |
| | G G D V V H L • |
| | GGGAGGAGAC GTGGTACACT |
| | CCCTCCTCTG CACCATGTGA |
| | NS2A |
| | ---------------------------------------------------------------------------------------- |
| | • A L M A T F K I Q P V F M V A S F L K A R W T N Q E |
| 5201 | TGGCGCTCAT GGCGACCTTC AAGATACAAC CAGTGTTTAT GGTGGCATCG TTTCTTAAAG CGAGATGGAC CAACCAGGAG |
| | ACCGCGAGTA CCGCTGGAAG TTCTATGTTG GTCACAAATA CCACCGTAGC AAAGAATTTC GCTCTACCTG GTTGGTCCTC |
| | NS2A |
| | --------------------- |
| | N I L L M L A • |
| | AACATTTTGT TGATGTTGGC |
| | TTGTAAAACA ACTACAACCG |
| | NS2A |
| | ---------------------------------------------------------------------------------------- |
| | • A V F F Q M A Y H D A R Q I L L W E I P D V L N S L A |
| 5301 | GGCTGTTTTC TTTCAAATGG CTTATCACGA TGCCCGCCAA ATTCTGCTCT GGGAGATCCC TGATGTGTTG AATTCACTGG |
| | CCGACAAAAG AAAGTTTACC GAATAGTGCT ACGGGCGGTT TAAGACGAGA CCCTCTAGGG ACTACACAAC TTAAGTGACC |
| | NS2A |
| | --------------------- |
| | I A W M I L |
| | CAATAGCTTG GATGATACTG |
| | GTTATCGAAC CTACTATGAC |
| | NS2A |
| | ---------------------------------------------------------------------------------------- |
| | R A I T F T T T S N V V V P L L A L L T P G L R C L N |
| 5401 | AGAGCCATAA CATTCACAAC GACATCAAAC GTGGTTGTTC CGCTGCTAGC CCTGCTAACA CCCGGGCTGA GATGCTTGAA |
| | TCTCGGTATT GTAAGTGTTG CTGTAGTTTG CACCAACAAG GCGACGATCG GGACGATTGT GGGCCCGACT CTACGAACTT |
| | NS2A |
| | --------------------- |
| | L D V Y R I L • |
| | TCTGGATGTG TACAGGATAC |
| | AGACCTACAC ATGTCCTATG |
| | NS2A |
| | ---------------------------------------------------------------------------------------- |
| | • L L M V G I G S L I R E K R S A A A K K K G A S L L |
| 5501 | TGCTGTTGAT GGTCGGAATA GGCAGCTTGA TCAGGGAGAA GAGGAGCGCA GCTGCAAAAA AGAAAGGAGC AAGTCTGCTA |
| | ACGACAACTA CCAGCCTTAT CCGTCGAACT AGTCCCTCTT CTCCTCGCGT CGACGTTTTT TCTTTCCTCG TTCAGACGAT |
| | NS2A |
| | --------------------- |
| | C L A L A S T • |
| | TGCTTGGCTC TAGCCTCAAC |
| | ACGAACCGAG ATCGGAGTTG |
| | NS2B |
| | ------------------ |
| | NS2A |
| | ---------------------------------------------------------------------- |
| | • G L F N P M I L A A G L I A C D P N R K R G W P A T E |
| 5601 | AGGACTCTTC AACCCCATGA TCCTTGCTGC TGGACTGATT GCATGTGATC CCAACCGTAA ACGCGGGTGG CCCGCAACTG |
| | TCCTGAGAAG TTGGGGTACT AGGAACGACG ACCTGACTAA CGTACACTAG GGTTGGCATT TGCGCCCACC GGGCGTTGAC |
| | NS2B |
| | --------------------- |
| | V M T A V G |
| | AAGTGATGAC AGCTGTCGGC |
| | TTCACTACTG TCGACAGCCG |
| | NS2B |
| | ---------------------------------------------------------------------------------------- |
| | L M F A I V G G L A E L D I D S M A I P M T I A G L M |
| 5701 | CTAATGTTTG CCATCGTCGG AGGGCTGGCA GAGCTTGACA TTGACTCCAT GGCCATTCCA ATGACTATCG CGGGGCTCAT |
| | GATTACAAAC GGTAGCAGCC TCCCGACCGT CTCGAACTGT AACTGAGGTA CCGGTAAGGT TACTGATAGC GCCCCGAGTA |
| | NS2B |
| | --------------------- |
| | F A A F V I S • |
| | GTTTGCTGCT TTCGTGATTT |
| | CAAACGACGA AAGCACTAAA |
| | NS2B |
| | ---------------------------------------------------------------------------------------- |
| | • G K S T D M W I E R T A D I S W E S D A E I T G S S |
| 5801 | CTGGGAAATC AACAGATATG TGGATTGAGA GAACGGCGGA CATTTCCTGG GAAAGTGATG CAGAGATTAC AGGCTCGAGC |
| | GACCCTTTAG TTGTCTATAC ACCTAACTCT CTTGCCGCCT GTAAAGGACC CTTTCACTAC GTCTCTAATG TCCGAGCTCG |
| | NS2B |
| | --------------------- |
| | E R V D V R L • |
| | GAAAGAGTTG ATGTGCGGCT |
| | CTTTCTCAAC TACACGCCGA |
| | NS2B |
| | ---------------------------------------------------------------------------------------- |
| | • D D D G N F Q L M N D P G A P W K I W M L R M V C L A |
| 5901 | TGATGATGAT GGAAACTTCC AGCTCATGAA TGATCCAGGA GCACCTTGGA AGATATGGAT GCCGAGTATG GTCTGTCTCG |
| | ACTACTACTA CCTTTGAAGG TCGAGTACTT ACTAGGTCCT CGTGGAACCT TCTATACCTA CGAGTCTTAC CAGACAGAGC |
| | NS2B |
| | --------------------- |
| | I S A Y T P |
| | CGATTAGTGC GTACACCCCC |
| | GCTAATCACG CATGTGGGGG |
| | NS3 |
| | -------------------------- |
| | NS2B |
| | -------------------------------------------------------------- |
| | W A I L P S V V G F W I T L Q Y T K R G G V L W D T P |
| 6001 | TGGGCAATCT TGCCCTCAGT AGTTGGATTT TGGATAACTC TCCAATACAC AAAGAGAGGA GGCGTGTTGT GGGACACTCC |
| | ACCCGTTAGA ACGGGAGTCA TCAACCTAAA ACCTATTGAG AGGTTATGTG TTTCTCTCCT CCGCACAACA CCCTGTGAGG |
| | NS3 |
| | --------------------- |
| | S P K E Y K K • |
| | CTCACCAAAG GAGTACAAAA |
| | GAGTGGTTTC CTCATGTTTT |
| | NS3 |
| | ---------------------------------------------------------------------------------------- |
| | • G D T T T G V Y R I M T R G L L G S Y Q A G A G V M |
| 6101 | AGGGGGACAC GACCACCGGC GTCTACAGGA TCATGACTCG TGGGCTGCTC GGCAGTTATC AAGCAGGAGC AGGCGTGATG |
| | TCCCCCTGTG CTGGTGGCCG CAGATGTCCT AGTACTGAGC ACCCGACGAG CCGTCAATAG TTCGTCCTCG TCCGCACTAC |
| | NS3 |
| | --------------------- |
| | V E G V F H T • |
| | GTTGAAGGTG TTTTCCACAC |
| | CAACTTCCAC AAAAGGTGTG |
| | NS3 |
| | ---------------------------------------------------------------------------------------- |
| | • L W H T T K G A A L M S G E G R L D P Y W G S V K E D |
| 6201 | CCTTTGGCAT ACAACAAAAG GAGCCGCTTT GATGAGCGGA GAGGGCCGCC TGGACCCATA CTGGGGCAGT GTCAAGGAGG |
| | GGAAACCGTA TGTTGTTTTC CTCGGCGAAA CTACTCGCCT CTCCCGGCGG ACCTGGGTAT GACCCCGTCA CAGTTCCTCC |
| | NS3 |
| | --------------------- |
| | R L C Y G G |
| | ATCGACTTTG TTACGGAGGA |
| | TAGCTGAAAC AATGCCTCCT |
| | NS3 |
| | ---------------------------------------------------------------------------------------- |
| | P W K L Q H K W N G Q D E V Q M I V V E P G K N V K N |
| 6301 | CCCTGGAAAT TGCAGCACAA GTGGAACGGG CAGGATGAGG TGCAGATGAT TGTGGTGGAA CCTGGCAAGA ACGTTAAGAA |
| | GGGACCTTTA ACGTCGTGTT CACCTTGCCC GTCCTACTCC ACGTCTACTA ACACCACCTT GGACCGTTCT TGCAATTCTT |
| | NS3 |
| | --------------------- |
| | V Q T K P G V • |
| | CGTCCAGACG AAACCAGGGG |
| | GCAGGTCTGC TTTGGTCCCC |
| | NS3 |
| | ---------------------------------------------------------------------------------------- |
| | • F K T P E G E I G A V T L D F P T G T S G S P I V D |
| 6401 | TGTTCAAAAC ACCTGAAGGA GAAATCGGGG CCGTGACTTT GGACTTCCCC ACTGGAACAT CAGGCTCACC AATAGTGGAC |
| | ACAAGTTTTG TGGACTTCCT CTTTAGCCCC GGCACTGAAA CCTGAAGGGG TGACCTTGTA GTCCGAGTGG TTATCACCTG |
| | NS3 |
| | --------------------- |
| | K N G D V I G • |
| | AAAAACGGTG ATGTGATTGG |
| | TTTTTGCCAC TACACTAACC |
| | NS3 |
| | ---------------------------------------------------------------------------------------- |
| | • L Y G N G V I M P N G S Y I S A I V Q G E R M D E P I |
| 6501 | GCTTTATGGC AATGGAGTCA TAATGCCCAA CGGCTCATAC ATAAGCGCGA TAGTGCAGGG TGAAAGGATG GATGAGCCAA |
| | CGAAATACCG TTACCTCAGT ATTACGGGTT GCCGAGTATG TATTCGCGCT ATCACGTCCC ACTTTCCTAC CTACTCGGTT |
| | NS3 |
| | --------------------- |
| | P A G F E P |
| | TCCCAGCCGG ATTCGAACCT |
| | AGGGTCGGCC TAAGCTTGGA |
| | NS3 |
| | ---------------------------------------------------------------------------------------- |
| | E M L R K K Q I T V L D L H P G A G K T R R I L P Q I |
| 6601 | GAGATGCTGA GGAAAAAACA GATCACTGTA CTGGATCTCC ATCCCGGCGC CGGTAAAACA AGGAGGATTC TGCCACAGAT |
| | CTCTACGACT CCTTTTTTGT CTAGTGACAT GACCTAGAGG TAGGGCCGCG GCCATTTTGT TCCTCCTAAG ACGGTGTCTA |
| | NS3 |
| | --------------------- |
| | I K E A I N R • |
| | CATCAAAGAG GCCATAAACA |
| | GTAGTTTCTC CGGTATTTGT |
| | NS3 |
| | ---------------------------------------------------------------------------------------- |
| | • R L R T A V L A P T R V V A A E M A E A L R G L P I |
| 6701 | GAAGACTGAG AACAGCCGTG CTAGCACCAA CCAGGGTTGT GGCTGCTGAG ATGGCTGAAG CACTGAGAGG ACTGCCCATC |
| | CTTCTGACTC TTGTCGGCAC GATCGTGGTT GGTCCCAACA CCGACGACTC TACCGACTTC GTGACTCTCC TGACGGGTAG |
| | NS3 |
| | --------------------- |
| | R Y Q T S A V • |
| | CGGTACCAGA CATCCGCAGT |
| | GCCATGGTCT GTAGGCGTCA |
| | NS3 |
| | ---------------------------------------------------------------------------------------- |
| | • P R E H N G N E I V D V M C H A T L T H R L M S P H R |
| 6801 | GCCCAGAGAA CATAATGGAA ATGAGATTGT TGATGTCATG TGTCATGCTA CCCTCACCCA CAGGCTGATG TCTCCTCACA |
| | CGGGTCTCTT GTATTACCTT TACTCTAACA ACTACAGTAC ACAGTACGAT GGGAGTGGGT GTCCGACTAC AGAGGAGTGT |
| | NS3 |
| | --------------------- |
| | V P N Y N L |
| | GGGTGCCGAA CTACAACCTG |
| | CCCACGGCTT GATGTTGGAC |
| | NS3 |
| | ---------------------------------------------------------------------------------------- |
| | F V M D E A H F T D P A S I A A R G Y I S T K V E L G |
| 6901 | TTCGTGATGG ATGAGGCTCA TTTCACCGAC CCAGCTAGCA TTGCAGCAAG AGGTTACATT TCCACAAAGG TCGAGCTAGG |
| | AAGCACTACC TACTCCGAGT AAAGTGGCTG GGTCGATCGT AACGTCGTTC TCCAATGTAA AGGTGTTTCC AGCTCGATCC |
| | NS3 |
| | --------------------- |
| | E A A A I F M • |
| | GGAGGCGGCG GCAATATTCA |
| | CCTCCGCCGC CGTTATAAGT |
| | NS3 |
| | ---------------------------------------------------------------------------------------- |
| | • T A T P P G T S D P F P E S N S P I S D L Q T E I P |
| 7001 | TGACAGCCAC CCCACCAGGC ACTTCAGATC CATTCCCAGA GTCCAATTCA CCAATTTCCG ACTTACAGAC TGAGATCCCG |
| | ACTGTCGGTG GGGTGGTCCG TGAAGTCTAG GTAAGGGTCT CAGGTTAAGT GGTTAAAGGC TGAATGTCTG ACTCTAGGGC |
| | NS3 |
| | --------------------- |
| | D R A W N S G • |
| | GATCGAGCTT GGAACTCTGG |
| | CTAGCTCGAA CCTTGAGACC |
| | NS3 |
| | ---------------------------------------------------------------------------------------- |
| | • Y E W I T E Y T G K T V W F V P S V K M G N E I A L C |
| 7101 | ATACGAATGG ATCACAGAAT ACACCGGGAA GACGGTTTGG TTTGTGCCTA GTGTTAAGAT GGGGAATGAG ATTGCCCTTT |
| | TATGCTTACC TAGTGTCTTA TGTGGCCCTT CTGCCAAACC AAACACGGAT CACAATTCTA CCCCTTACTC TAACGGGAAA |
| | NS3 |
| | --------------------- |
| | L Q R A G K |
| | GCCTACAACG TGCTGGAAAG |
| | CGGATGTTGC ACGACCTTTC |
| | NS3 |
| | ---------------------------------------------------------------------------------------- |
| | K V V Q L N R K S Y E T E Y P K C K N D D W D F V I T |
| 7201 | AAAGTAGTCC AATTGAACAG AAAGTCGTAC GAGACGGAGT ACCCAAAATG TAAGAACGAT GATTGGGACT TTGTTATCAC |
| | TTTCATCAGG TTAACTTGTC TTTCAGCATG CTCTGCCTCA TGGGTTTTAC ATTCTTGCTA CTAACCCTGA AACAATAGTG |
| | NS3 |
| | --------------------- |
| | T D I S E M G • |
| | AACAGACATA TCTGAAATGG |
| | TTGTCTGTAT AGACTTTACC |
| | NS3 |
| | ---------------------------------------------------------------------------------------- |
| | • A N F K A S R V I D S R K S V K P T I I T E G E G R |
| 7301 | GGGCTAACTT CAAGGCGAGC AGGGTGATTG ACAGCCGGAA GAGTGTGAAA CCAACCATCA TAACAGAAGG AGAAGGGAGA |
| | CCCGATTGAA GTTCCGCTCG TCCCACTAAC TGTCGGCCTT CTCACACTTT GGTTGGTAGT ATTGTCTTCC TCTTCCCTCT |
| | NS3 |
| | --------------------- |
| | V I L G E P S • |
| | GTGATCCTGG GAGAACCATC |
| | CACTAGGACC CTCTTGGTAG |
| | NS3 |
| | ---------------------------------------------------------------------------------------- |
| | • A V T A A S A A Q R R G R I G R N P S Q V G D E Y C Y |
| 7401 | TGCAGTGACA GCAGCTAGTG CCGCCCAGAG ACGTGGACGT ATCGGTAGAA ATCCGTCGCA AGTTGGTGAT GAGTACTGTT |
| | ACGTCACTGT CGTCGATCAC GGCGGGTCTC TGCACCTGCA TAGCCATCTT TAGGCAGCGT TCAACCACTA CTCATGACAA |
| | NS3 |
| | --------------------- |
| | G G H T N E |
| | ATGGGGGGCA CACGAATGAA |
| | TACCCCCCGT GTGCTTACTT |
| | NS3 |
| | ---------------------------------------------------------------------------------------- |
| | D D S N F A H W T E A R I M L D N I N M P N G L I A Q |
| 7501 | GACGACTCGA ACTTCGCCCA TTGGACTGAG GCACGAATCA TGCTGGACAA CATCAACATG CCAAACGGAC TGATCGCTCA |
| | CTGCTGAGCT TGAAGCGGGT AACCTGACTC CGTGCTTAGT ACGACCTGTT GTAGTTGTAC GGTTTGCCTG ACTAGCGAGT |
| | NS3 |
| | --------------------- |
| | F Y Q P E R E • |
| | ATTCTACCAA CCAGAGCGTG |
| | TAAGATGGTT GGTCTCGCAC |
| | NS3 |
| | ---------------------------------------------------------------------------------------- |
| | • K V Y T M D G E Y R L R G E E R K N F L E L L R T A |
| 7601 | AGAAGGTATA TACCATGGAT GGGGAATACC GGCTCAGAGG AGAAGAGAGA AAAAACTTTC TGGAACTGTT GAGGACTGCA |
| | TCTTCCATAT ATGGTACCTA CCCCTTATGG CCGAGTCTCC TCTTCTCTCT TTTTTGAAAG ACCTTGACAA CTCCTGACGT |
| | NS3 |
| | --------------------- |
| | D L P V W L A • |
| | GATCTGCCAG TTTGGCTGGC |
| | CTAGACGGTC AAACCGACCG |
| | NS3 |
| | ---------------------------------------------------------------------------------------- |
| | • Y K V A A A G V S Y H D R R W C F D G P R T N T I L E |
| 7701 | TTACAAGGTT GCAGCGGCTG GAGTGTCATA CCACGACCGG AGGTGGTGCT TTGATGGTCC TAGGACAAAC ACAATTTTAG |
| | AATGTTCCAA CGTCGCCGAC CTCACAGTAT GGTGCTGGCC TCCACCACGA AACTACCAGG ATCCTGTTTG TGTTAAAATC |
| | NS3 |
| | --------------------- |
| | D N N E V E |
| | AAGACAACAA CGAAGTGGAA |
| | TTCTGTTGTT GCTTCACCTT |
| | NS3 |
| | ---------------------------------------------------------------------------------------- |
| | V I T K L G E R K I L R P R W I D A R V Y S D H Q A L |
| 7801 | GTCATCACGA AGCTTGGTGA AAGGAAGATT CTGAGGCCGC GCTGGATTGA CGCCAGGGTG TACTCGGATC ACCAGGCACT |
| | CAGTAGTGCT TCGAACCACT TTCCTTCTAA GACTCCGGCG CGACCTAACT GCGGTCCCAC ATGAGCCTAG TGGTCCGTGA |
| | NS3 |
| | --------------------- |
| | K A F K D F A • |
| | AAAGGCGTTC AAGGACTTCG |
| | TTTCCGCAAG TTCCTGAAGC |
| | NS4A |
| | ------------------------------------------------------------------------- |
| | NS3 |
| | --------------- |
| | • S G K R S Q I G L I E V L G K M P E H F M G K T W E |
| 7901 | CCTCGGGAAA ACGTTCTCAG ATAGGGCTCA TTGAGGTTCT GGGAAAGATG CCTGAGCACT TCATGGGGAA GACATGGGAA |
| | GGAGCCCTTT TGCAAGAGTC TATCCCGAGT AACTCCAAGA CCCTTTCTAC GGACTCGTGA AGTACCCCTT CTGTACCCTT |
| | NS4A |
| | --------------------- |
| | A L D T M Y V • |
| | GCACTTGACA CCATGTACGT |
| | CGTGAACTGT GGTACATGCA |
| | NS4A |
| | ---------------------------------------------------------------------------------------- |
| | • V A T A E K G G R A H R M A L E E L P D A L Q T I A L |
| 8001 | TGTGGCCACT GCAGAGAAAG GAGGAAGAGC TCACAGAATG GCCCTGGAGG AACTGCCAGA TGCTCTTCAG ACAATTGCCT |
| | ACACCGGTGA CGTCTCTTTC CTCCTTCTCG AGTGTCTTAC CGGGACCTCC TTGACGGTCT ACGAGAAGTC TGTTAACGGA |
| | NS4A |
| | --------------------- |
| | I A L L S V |
| | TGATTGCCTT ATTGAGTGTG |
| | ACTAACGGAA TAACTCACAC |
| | NS4A |
| | ---------------------------------------------------------------------------------------- |
| | M T M G V F F L L M Q R K G I G K I G L G G A V L G V |
| 8101 | ATGACCATGG GAGTATTCTT CCTCCTCATG CAGCGGAAGG GCATTGGAAA GATAGGTTTG GGAGGCGCTG TCTTGGGAGT |
| | TACTGGTACC CTCATAAGAA GGAGGAGTAC GTCGCCTTCC CGTAACCTTT CTATCCAAAC CCTCCGCGAC AGAACCCTCA |
| | NS4A |
| | --------------------- |
| | A T F F C W M • |
| | CGCGACCTTT TTCTGTTGGA |
| | GCGCTGGAAA AAGACAACCT |
| | NS4A |
| | ---------------------------------------------------------------------------------------- |
| | • A E V P G T K I A G M L L L S L L L M I V L I P E P |
| 8201 | TGGCTGAAGT TCCAGGAACG AAGATCGCCG GAATGTTGCT GCTCTCCCTT CTCTTGATGA TTGTGCTAAT TCCTGAGCCA |
| | ACCGACTTCA AGGTCCTTGC TTCTAGCGGC CTTACAACGA CGAGAGGGAA GAGAACTACT AACACGATTA AGGACTCGGT |
| | NS4A |
| | --------------------- |
| | E K Q R S Q T • |
| | GAGAAGCAAC GTTCGCAGAC |
| | CTCTTCGTTG CAAGCGTCTG |
| | NS4B |
| | --------------------- |
| | NS4A |
| | ------------------------------------------------------------------- |
| | • D N Q L A V F L I C V M T L V S A V A A N E M G W L D |
| 8301 | AGACAACCAG CTAGCCGTGT TCCTGATATG TGTCATGACC CTTGTGAGCG CAGTGGCAGC CAACGAGATG GGTTGGCTAG |
| | TCTGTTGGTC GATCGGCACA AGGACTATAC ACAGTACTGG GAACACTCGC GTCACCGTCG GTTGCTCTAC CCAACCGATC |
| | NS4B |
| | --------------------- |
| | K T K S D I |
| | ATAAGACCAA GAGTGACATA |
| | TATTCTGGTT CTCACTGTAT |
| | NS4B |
| | ---------------------------------------------------------------------------------------- |
| | S S L F G Q R I E V K E N F S M G E F L L D L R P A T |
| 8401 | AGCAGTTTGT TTGGGCAAAG AATTGAGGTC AAGGAGAATT TCAGCATGGG AGAGTTTCTT CTGGACTTGA GGCCGGCAAC |
| | TCGTCAAACA AACCCGTTTC TTAACTCCAG TTCCTCTTAA AGTCGTACCC TCTCAAAGAA GACCTGAACT CCGGCCGTTG |
| | NS4B |
| | --------------------- |
| | A W S L Y A V • |
| | AGCCTGGTCA CTGTACGCTG |
| | TCGGACCAGT GACATGCGAC |
| | NS4B |
| | ---------------------------------------------------------------------------------------- |
| | • T T A V L T P L L K H L I T S D Y I N T S L T S I N |
| 8501 | TGACAACAGC GGTCCTCACT CCACTGCTAA AGCATTTGAT CACGTCAGAT TACATCAACA CCTCATTGAC CTCAATAAAC |
| | ACTGTTGTCG CCAGGAGTGA GGTGACGATT TCGTAAACTA GTGCAGTCTA ATGTAGTTGT GGAGTAACTG GAGTTATTTG |
| | NS4B |
| | --------------------- |
| | V Q A S A L F • |
| | GTTCAGGCAA GTGCACTATT |
| | CAAGTCCGTT CACGTGATAA |
| | NS4B |
| | ---------------------------------------------------------------------------------------- |
| | • T L A R G F P F V D V G V S A L L L A A G C W G Q V T |
| 8601 | CACACTCGCG CGAGGCTTCC CCTTCGTCGA TGTTGGAGTG TCGGCTCTCC TGCTAGCAGC CGGATGCTGG GGACAAGTCA |
| | GTGTGAGCGC GCTCCGAAGG GGAAGCAGCT ACAACCTCAC AGCCGAGAGG ACGATCGTCG GCCTACGACC CCTGTTCAGT |
| | NS4B |
| | --------------------- |
| | L T V T V T |
| | CCCTCACCGT TACGGTAACA |
| | GGGAGTGGCA ATGCCATTGT |
| | NS4B |
| | ---------------------------------------------------------------------------------------- |
| | A A T L L F C H Y A Y M V P G W Q A E A M R S A Q R R |
| 8701 | GCGGCAACAC TCCTTTTTTG CCACTATGCC TACATGGTTC CCGGTTGGCA AGCTGAGGCA ATGCGCTCAG CCCAGCGGCG |
| | CGCCGTTGTG AGGAAAAAAC GGTGATACGG ATGTACCAAG GGCCAACCGT TCGACTCCGT TACGCGAGTC GGGTCGCCGC |
| | NS4B |
| | --------------------- |
| | T A A G I M K • |
| | GACAGCGGCC GGAATCATGA |
| | CTGTCGCCGG CCTTAGTACT |
| | NS4B |
| | ---------------------------------------------------------------------------------------- |
| | • N A V V D G I V A T D V P E L E R T T P I M Q K K I |
| 8801 | AGAACGCTGT AGTGGATGGC ATCGTGGCCA CGGACGTCCC AGAATTAGAG CGCACCACAC CCATCATGCA GAAGAAAATT |
| | TCTTGCGACA TCACCTACCG TAGCACCGGT GCCTGCAGGG TCTTAATCTC GCGTGGTGTG GGTAGTACGT CTTCTTTTAA |
| | NS4B |
| | --------------------- |
| | G Q I M L I L • |
| | GGACAGATCA TGCTGATCTT |
| | CCTGTCTAGT ACGACTAGAA |
| | NS4B |
| | ---------------------------------------------------------------------------------------- |
| | • V S L A A V V V N P S V K T V R E A G I L I T A A A V |
| 8901 | GGTGTCTCTA GCTGCAGTAG TAGTGAACCC GTCTGTGAAG ACAGTACGAG AAGCCGGAAT TTTGATCACG GCCGCAGCGG |
| | CCACAGAGAT CGACGTCATC ATCACTTGGG CAGACACTTC TGTCATGCTC TTCGGCCTTA AAACTAGTGC CGGCGTCGCC |
| | NS4B |
| | --------------------- |
| | T L W E N G |
| | TGACGCTTTG GGAGAATGGA |
| | ACTGCGAAAC CCTCTTACCT |
| | NS4B |
| | ---------------------------------------------------------------------------------------- |
| | A S S V W N A T T A I G L C H I M R G G W L S C L S I |
| 9001 | GCAAGCTCTG TTTGGAACGC AACAACTGCC ATCGGACTCT GCCACATCAT GCGTGGGGGT TGGTTGTCAT GTCTATCCAT |
| | CGTTCGAGAC AAACCTTGCG TTGTTGACGG TAGCCTGAGA CGGTGTAGTA CGCACCCCCA ACCAACAGTA CAGATAGGTA |
| | NS4B |
| | --------------------- |
| | T W T L I K N • |
| | AACATGGACA CTCATAAAGA |
| | TTGTACCTGT GAGTATTTCT |
| | NS5 |
| | ------------------------------------------------------------ |
| | NS4B |
| | ---------------------------- |
| | • M E K P G L K R G G A K G R T L G E V W K E R L N Q |
| 9101 | ACATGGAAAA ACCAGGACTA AAAAGAGGTG GGGCAAAAGG ACGCACCTTG GGAGAGGTTT GGAAAGAAAG ACTCAACCAG |
| | TGTACCTTTT TGGTCCTGAT TTTTCTCCAC CCCGTTTTCC TGCGTGGAAC CCTCTCCAAA CCTTTCTTTC TGAGTTGGTC |
| | NS5 |
| | --------------------- |
| | M T K E E F T • |
| | ATGACAAAAG AAGAGTTCAC |
| | TACTGTTTTC TTCTCAAGTG |
| | NS5 |
| | ---------------------------------------------------------------------------------------- |
| | • R Y R K E A I I E V D R S A A K H A R K E G N V T G G |
| 9201 | TAGGTACCGC AAAGAGGCCA TCATCGAAGT CGATCGCTCA GCGGCAAAAC ACGCCAGGAA AGAAGGCAAT GTCACTGGAG |
| | ATCCATGGCG TTTCTCCGGT AGTAGCTTCA GCTAGCGAGT CGCCGTTTTG TGCGGTCCTT TCTTCCGTTA CAGTGACCTC |
| | NS5 |
| | --------------------- |
| | H P V S R G |
| | GGCATCCAGT CTCTAGGGGC |
| | CCGTAGGTCA GAGATCCCCG |
| | NS5 |
| | ---------------------------------------------------------------------------------------- |
| | T A K L R W L V E R R F L E P V G K V I D L G C G R G |
| 9301 | ACAGCAAAAC TGAGATGGCT GGTCGAACGG AGGTTTCTCG AACCGGTCGG AAAAGTGATT GACCTTGGAT GTGGAAGAGG |
| | TGTCGTTTTG ACTCTACCGA CCAGCTTGCC TCCAAAGAGC TTGGCCAGCC TTTTCACTAA CTGGAACCTA CACCTTCTCC |
| | NS5 |
| | --------------------- |
| | G W C Y Y M A • |
| | CGGTTGGTGT TACTATATGG |
| | GCCAACCACA ATGATATACC |
| | NS5 |
| | ---------------------------------------------------------------------------------------- |
| | • T Q K R V Q E V R G Y T K G G P G H E E P Q L V Q S |
| 9401 | CAACCCAAAA AAGAGTCCAA GAAGTCAGAG GGTACACAAA GGGCGGTCCC GGACATGAAG AGCCCCAACT AGTGCAAAGT |
| | GTTGGGTTTT TTCTCAGGTT CTTCAGTCTC CCATGTGTTT CCCGCCAGGG CCTGTACTTC TCGGGGTTGA TCACGTTTCA |
| | NS5 |
| | --------------------- |
| | Y G W N I V T • |
| | TATGGATGGA ACATTGTCAC |
| | ATACCTACCT TGTAACAGTG |
| | NS5 |
| | ---------------------------------------------------------------------------------------- |
| | • M K S G V D V F Y R P S E C C D T L L C D I G E S S S |
| 9501 | CATGAAGAGT GGAGTGGATG TGTTCTACAG ACCTTCTGAG TGTTGTGACA CCCTCCTTTG TGACATCGGA GAGTCCTCGT |
| | GTACTTCTCA CCTCACCTAC ACAAGATGTC TGGAAGACTC ACAACACTGT GGGAGGAAAC ACTGTAGCCT CTCAGGAGCA |
| | NS5 |
| | --------------------- |
| | S A E V E E |
| | CAAGTGCTGA GGTTGAAGAG |
| | GTTCACGACT CCAACTTCTC |
| | NS5 |
| | ---------------------------------------------------------------------------------------- |
| | H R T I R V L E M V E D W L H R G P R E F C V K V L C |
| 9601 | CATAGGACGA TTCGGGTCCT TGAAATGGTT GAGGACTGGC TGCACCGAGG GCCAAGGGAA TTTTGCGTGA AGGTGCTCTG |
| | GTATCCTGCT AAGCCCAGGA ACTTTACCAA CTCCTGACCG ACGTGGCTCC CGGTTCCCTT AAAACGCACT TCCACGAGAC |
| | NS5 |
| | --------------------- |
| | P Y M P K V I • |
| | CCCCTACATG CCGAAAGTCA |
| | GGGGATGTAC GGCTTTCAGT |
| | NS5 |
| | ---------------------------------------------------------------------------------------- |
| | • E K M E L L Q R R Y G G G L V R N P L S R N S T H E |
| 9701 | TAGAGAAGAT GGAGCTGCTC CAACGCCGGT ATGGGGGGGG ACTGGTCAGA AACCCACTCT CACGGAATTC CACGCACGAG |
| | ATCTCTTCTA CCTCGACGAG GTTGCGGCCA TACCCCCCCC TGACCAGTCT TTGGGTGAGA GTGCCTTAAG GTGCGTGCTC |
| | NS5 |
| | --------------------- |
| | M Y W V S R A • |
| | ATGTATTGGG TGAGTCGAGC |
| | TACATAACCC ACTCAGCTCG |
| | NS5 |
| | ---------------------------------------------------------------------------------------- |
| | • S G N V V H S V N M T S Q V L L G R M E K R T W K G P |
| 9801 | TTCAGGCAAT GTGGTACATT CAGTGAATAT GACCAGCCAG GTGCTCCTAG GAAGAATGGA AAAAAGGACC TGGAAGGGAC |
| | AAGTCCGTTA CACCATGTAA GTCACTTATA CTGGTCGGTC CACGAGGATC CTTCTTACCT TTTTTCCTGG ACCTTCCCTG |
| | NS5 |
| | --------------------- |
| | Q Y E E D V |
| | CCCAATACGA GGAAGACGTA |
| | GGGTTATGCT CCTTCTGCAT |
| | NS5 |
| | ---------------------------------------------------------------------------------------- |
| | N L G S G T R A V G K P L L N S D T S K I K N R I E R |
| 9901 | AACTTGGGAA GTGGAACCAG GGCGGTGGGA AAACCCCTGC TCAACTCAGA CACCAGTAAA ATCAAGAACA GGATTGAACG |
| | TTGAACCCTT CACCTTGGTC CCGCCACCCT TTTGGGGACG AGTTGAGTCT GTGGTCATTT TAGTTCTTGT CCTAACTTGC |
| | NS5 |
| | --------------------- |
| | L R R E Y S S • |
| | ACTCAGGCGT GAGTACAGTT |
| | TGAGTCCGCA CTCATGTCAA |
| | NS5 |
| | ---------------------------------------------------------------------------------------- |
| | • T W H H D E N H P Y R T M N Y H G S Y D V K P T G S |
| 10001 | CGACGTGGCA CCACGATGAG AACCACCCAT ATAGAACCTG GAACTATCAT GGCAGTTATG ATGTGAAGCC CACAGGCTCC |
| | GCTGCACCGT GGTGCTACTC TTGGTGGGTA TATCTTGGAC CTTGATAGTA CCGTCAATAC TACACTTCGG GTGTCCGAGG |
| | NS5 |
| | --------------------- |
| | A S S L V N G • |
| | GCCAGTTCGC TGGTCAATGG |
| | CGGTCAAGCG ACCAGTTACC |
| | NS5 |
| | ---------------------------------------------------------------------------------------- |
| | • V V R L L S K P W D T I T N V T T M A M T D T T P F G |
| 10101 | AGTGGTCAGG CTCCTCTCAA AACCATGGGA CACCATCACG AATGTTACCA CCATGGCCAT GACTGACACT ACTCCCTTCG |
| | TCACCAGTCC GAGGAGAGTT TTGGTACCCT GTGGTAGTGC TTACAATGGT GGTACCGGTA CTGACTGTGA TGAGGGAAGC |
| | NS5 |
| | --------------------- |
| | Q Q R V F K |
| | GGCAGCAGCG AGTGTTCAAA |
| | CCGTCGTCGC TCACAAGTTT |
| | NS5 |
| | ---------------------------------------------------------------------------------------- |
| | E K V D T K A P E P P E G V K Y V L N E T T N W L W A |
| 10201 | GAGAAGGTGG ACACGAAAGC TCCTGAACCG CCAGAAGGAG TGAAGTACGT GCTCAACGAG ACCACCAACT GGTTGTGGGC |
| | CTCTTCCACC TGTGCTTTCG AGGACTTGGC GGTCTTCCTC ACTTCATGCA CGAGTTGCTC TGGTGGTTGA CCAACACCCG |
| | NS5 |
| | --------------------- |
| | F L A R E K R • |
| | GTTTTTGGCC AGAGAAAAAC |
| | CAAAAACCGG TCTCTTTTTG |
| | NS5 |
| | ---------------------------------------------------------------------------------------- |
| | • P R M C S R E E F I R K V N S N A A L G A M F E E Q |
| 10301 | GTCCCAGAAT GTGCTCTCGA GAGGAATTCA TAAGAAAGGT CAACAGCAAT GCAGCTTTGG GTGCCATGTT TGAAGAGCAG |
| | CAGGGTCTTA CACGAGAGCT CTCCTTAAGT ATTCTTTCCA GTTGTCGTTA CGTCGAAACC CACGGTACAA ACTTCTCGTC |
| | NS5 |
| | --------------------- |
| | N Q W R S A R • |
| | AATCAATGGA GGAGCGCCAG |
| | TTAGTTACCT CCTCGCGGTC |
| | NS5 |
| | ---------------------------------------------------------------------------------------- |
| | • E A V E D P K F W E M V D E E R E A H L R G E C H T C |
| 10401 | AGAAGGAGTT GAAGATCCAA AATTTTGGGA AATGGTGGAT GAGGAGCGCG AGGCACATCT GCGGGGGGAA TGTCACACTT |
| | TCTTCGTCAA CTTCTAGGTT TTAAAACCCT TTACCACCTA CTCCTCGCGC TCCGTGTAGA CGCCCCCCTT ACAGTGTGAA |
| | NS5 |
| | --------------------- |
| | I Y N M M G |
| | GCATTTACAA CATGATGGGA |
| | CGTAAATGTT GTACTACCCT |
| | NS5 |
| | ---------------------------------------------------------------------------------------- |
| | K R E K K P G E F G K A K G S R A I W F M W L G A R F |
| 10501 | AAGAGAGAGA AAAAACCCGG AGAGTTCGGA AAGGCCAAGG GAAGCAGAGC CATTTGGTTC ATGTGGCTCG GAGCTCGCTT |
| | TTCTCTCTCT TTTTTGGGCC TCTCAAGCCT TTCCGGTTCC CTTCGTCTCG GTAAACCAAG TACACCGAGC CTCGAGCGAA |
| | NS5 |
| | --------------------- |
| | L E F E A L G • |
| | TCTGGAGTTC GAGGCTCTGG |
| | AGACCTCAAG CTCCGAGACC |
| | NS5 |
| | ---------------------------------------------------------------------------------------- |
| | • F L N E D H W L G R K N S G G G V E G L G L Q K L G |
| 10601 | GTTTTCTCAA TGAAGACCAC TGGCTTGGAA GAAAGAACTC AGGAGGAGGT GTCGAGGGCT TGGGCCTCCA AAAACTGGGT |
| | CAAAAGAGTT ACTTCTGGTG ACCGAACCTT CTTTCTTGAG TCCTCCTCCA CAGCTCCCGA ACCCGGAGGT TTTTGACCCA |
| | NS5 |
| | --------------------- |
| | Y I L R E V G • |
| | TACATCCTGC GTGAAGTTGG |
| | ATGTAGGACG CACTTCAACC |
| | NS5 |
| | ---------------------------------------------------------------------------------------- |
| | • I R P G G K I Y A D D T A G W D T R I T R A D L E N E |
| 10701 | CATCCGGCCT GGGGGCAAGA TCTATGCTGA TGACACAGCT GGCTGGGACA CCCGCATCAC GAGAGCTGAC TTGGAAAATG |
| | GTAGGCCGGA CCCCCGTTCT AGATACGACT ACTGTGTCGA CCGACCCTGT GGGCGTAGTG CTCTCGACTG AACCTTTTAC |
| | NS5 |
| | --------------------- |
| | A K V L E L |
| | AAGCTAAGGT GCTTGAGCTG |
| | TTCGATTCCA CGAACTCGAC |
| | NS5 |
| | ---------------------------------------------------------------------------------------- |
| | L D G E H R R L A R A I I E L T Y R H K V V K V M R P |
| 10801 | CTTGATGGGG AACATCGGCG TCTTGCCAGG GCCATCATTG AGCTCACCTA TCGTCACAAA GTTGTGAAAG TGATGCGCCC |
| | GAACTACCCC TTGTAGCCGC AGAACGGTCC CGGTAGTAAC TCGAGTGGAT AGCAGTGTTT CAACACTTTC ACTACGCGGG |
| | NS5 |
| | --------------------- |
| | A A D G R T V • |
| | GGCTGCTGAT GGAAGAACCG |
| | CCGACGACTA CCTTCTTGGC |
| | NS5 |
| | ---------------------------------------------------------------------------------------- |
| | • M D V I S R E D Q R G S G Q V V T Y A L N T F T N L |
| 10901 | TTATGGATGT TATCTCCAGA GAAGATCAGA GGGGGAGTGG ACAAGTTGTC ACCTACGCCC TAAACACTTT CACCAACCTG |
| | AATACCTACA ATAGAGGTCT CTTCTAGTCT CCCCCTCACC TGTTCAACAG TGGATGCGGG ATTTGTGAAA GTGGTTGGAC |
| | NS5 |
| | --------------------- |
| | A V Q L V R M • |
| | GCTGTCCAGC TGGTGAGGAT |
| | CGACAGGTCG ACCACTCCTA |
| | NS5 |
| | ---------------------------------------------------------------------------------------- |
| | • M E G E G V I G P D D V E K L T K G K G P K V R T W L |
| 11001 | GATGGAAGGG GAAGGAGTGA TTGGCCCAGA TGATGTGGAG AAACTCACAA AAGGGAAAGG ACCCAAAGTC AGGACCTGGC |
| | CTACCTTCCC CTTCCTCACT AACCGGGTCT ACTACACCTC TTTGAGTGTT TTCCCTTTCC TGGGTTTCAG TCCTGGACCG |
| | NS5 |
| | --------------------- |
| | F E N G E E |
| | TGTTTGAGAA TGGGGAAGAA |
| | ACAAACTCTT ACCCCTTCTT |
| | NS5 |
| | ---------------------------------------------------------------------------------------- |
| | R L S R M A V S G D D C V V K P L D D R F A T S L H F |
| 11101 | AGACTCAGCC GCATGGCTGT CAGTGGAGAT GACTGTGTGG TAAAGCCCCT GGACGATCGC TTTGCCACCT CGCTCCACTT |
| | TCTGAGTCGG CGTACCGACA GTCACCTCTA CTGACACACC ATTTCGGGGA CCTGCTAGCG AAACGGTGGA GCGAGGTGAA |
| | NS5 |
| | --------------------- |
| | L N A M S K V • |
| | CCTCAATGCT ATGTCAAAGG |
| | GGAGTTACGA TACAGTTTCC |
| | NS5 |
| | ---------------------------------------------------------------------------------------- |
| | • R K D I Q E W K P S T G W Y D W Q Q V P F C S N H F |
| 11201 | TTCGCAAAGA CATCCAAGAG TGGAAACCGT CAACTGGATG GTATGATTGG CAGCAGGTTC CATTTTGCTC AAACCATTTC |
| | AAGCGTTTCT GTAGGTTCTC ACCTTTGGCA GTTGACCTAC CATACTAACC GTCGTCCAAG GTAAAACGAG TTTGGTAAAG |
| | NS5 |
| | --------------------- |
| | T E L I M K D • |
| | ACTGAATTGA TCATGAAAGA |
| | TGACTTAACT AGTACTTTCT |
| | NS5 |
| | ---------------------------------------------------------------------------------------- |
| | • G R T L V V P C R G Q D E L V G R A R I S P G A G W N |
| 11301 | TGGAAGAACA CTGGTGGTTC CATGCCGAGG ACAGGATGAA TTGGTAGGCA GAGCTCGCAT ATCTCCAGGG GCCGGATGGA |
| | ACCTTCTTGT GACCACCAAG GTACGGCTCC TGTCCTACTT AACCATCCGT CTCGAGCGTA TAGAGGTCCC CGGCCTACCT |
| | NS5 |
| | --------------------- |
| | V R D T A C |
| | ACGTCCGCGA CACTGCTTGT |
| | TGCAGGCGCT GTGACGAACA |
| | NS5 |
| | ---------------------------------------------------------------------------------------- |
| | L A K S Y A Q M W L L L Y F H R R D L R L M A N A I C |
| 11401 | CTGGCTAAGT CTTATGCCCA GATGTGGCTG CTTCTGTACT TCCACAGAAG AGACCTGCGG CTCATGGCCA ACGCCATTTG |
| | GACCGATTCA GAGTACCGGT CTACACCGAC GAAGACATGA AGGTGTCTTC TCTGGACGCC GAGTACCGGT TGCGGTAAAC |
| | NS5 |
| | --------------------- |
| | S A V P V N W • |
| | CTCCGCTGTC CCTGTGAATT |
| | GAGGCGACAG GGACACTTAA |
| | NS5 |
| | ---------------------------------------------------------------------------------------- |
| | • V P T G R T T W S I H A G G E W M T T E D M L E V W |
| 11501 | GGGTCCCTAC CGGAAGAACC ACGTGGTCCA TCCATGCAGG AGGAGAGTGG ATGACAACAG AGGACATGTT GGAGGTCTGG |
| | CCCAGGGATG GCCTTCTTGG TGCACCAGGT AGGTACGTCC TCCTCTCACC TACTGTTGTC TCCTGTACAA CCTCCAGACC |
| | NS5 |
| | --------------------- |
| | N R V W I E E • |
| | AACCGTGTTT GGATAGAGGA |
| | TTGGCACAAA CCTATCTCCT |
| | NS5 |
| | ---------------------------------------------------------------------------------------- |
| | • N E W M E D K T P V E K W S D V P Y S G K R E D I W C |
| 11601 | GAATGAATGG ATGGAAGACA AAACCCCAGT GGAGAAATGG AGTGACGTCC CATATTCAGG AAAACGAGAG GACATCTGGT |
| | CTTACTTACC TACCTTCTGT TTTGGGGTCA CCTCTTTACC TCACTGCAGG GTATAAGTCC TTTTGCTCTC CTGTAGACCA |
| | NS5 |
| | --------------------- |
| | G S L I G T |
| | GTGGCAGCCT GATTGGCACA |
| | CACCGTCGGA CTAACCGTGT |
| | NS5 |
| | ---------------------------------------------------------------------------------------- |
| | R A R A T W A E N I Q V A I N Q V R A I I G D E K Y V |
| 11701 | AGAGCCCGAG CCACGTGGGC AGAAAACATC CAGGTGGCTA TCAACCAAGT CAGAGCAATC ATCGGAGATG AGAAGTATGT |
| | TCTCGGGCTC GGTGCACCCG TCTTTTGTAG GTCCACCGAT AGTTGGTTCA GTCTCGTTAG TAGCCTCTAC TCTTCATACA |
| | NS5 |
| | --------------------- |
| | D Y M S S L K • |
| | GGATTACATG AGTTCACTAA |
| | CCTAATGTAC TCAAGTGATT |
| | 3′ UTR |
| | ------------------------------------------- |
| | NS5 |
| | --------------------------------------------- |
| | • R Y E D T T L V E D T V L |
| 11801 | AGAGATATGA AGACACAACT TTGGTTGAGG ACACAGTACT GTAGATATTT AATCAATTGT AAATAGACAA TATAAGTATG |
| | TCTCTATACT TCTGTGTTGA AACCAACTCC TGTGTCATGA CATCTATAAA TTAGTTAACA TTTATCTGTT ATATTCATAC |
| | 3′ UTR |
| | --------------------- |
| | CATAAAAGTG TAGTTTTATA |
| | GTATTTTCAC ATCAAAATAT |
| | 3′ UTR |
| | ---------------------------------------------------------------------------------------- |
| 11901 | GTAGTATTTA GTGGTGTTAG TGTAAATAGT TAAGAAAATC TTGAGGAGAA AGTCAGGCCG GGAAGTTCCC GCCACCGGAA |
| | CATCATAAAT CACCACAATC ACATTTATCA ATTCTTTTAG AACTCCTCTT TCAGTCCGGC CCTTCAAGGG CGGTGGCCTT |
| | 3′ UTR |
| | --------------------- |
| | GTTGAGTAGA CGGTGCTGCC |
| | CAACTCATCT GCCACGACGG |
| | 3′ UTR |
| | ---------------------------------------------------------------------------------------- |
| 12001 | TGCGACTCAA CCCCAGGAGG ACTGGGTGAA CAAAGCCGCG AAGTGATCCA TGTAAGCCCT CAGAACCGTC TCGGAAGGAG |
| | ACGCTGAGTT GGGGTCCTCC TGACCCACTT GTTTCGGCGC TTCACTAGGT ACATTCGGGA GTCTTGGCAG AGCCTTCCTC |
| | 3′ UTR |
| | --------------------- |
| | GACCCCACAT GTTGTAACTT |
| | CTGGGGTGTA CAACATTGAA |
| | 3′ UTR |
| | ---------------------------------------------------------------------------------------- |
| 12101 | CAAAGCCCAA TGTCAGACCA CGCTACGGCG TGCTACTCTG CGGAGAGTGC AGTCTGCGAT AGTGCCCCAG GAGGACTGGG |
| | GTTTCGGGTT ACAGTCTGGT GCGATGCCGC ACGATGAGAC GCCTCTCACG TCAGACGCTA TCACGGGGTC CTCCTGACCC |
| | 3′ UTR |
| | --------------------- |
| | TTAACAAAGG CAAACCAACG |
| | AATTGTTTCC GTTTGGTTGC |
| | 3′ UTR |
| | ---------------------------------------------------------------------------------------- |
| 12201 | CCCCACGCGG CCCAAGCCCC GGTAATGGTG TTAACCAGGG CGAAAGGACT AGAGGTTAGA GGAGACCCCG CGGTTTAAAG |
| | GGGGTGCGCC GGGTTCGGGG CCATTACCAC AATTGGTCCC GCTTTCCTGA TCTCCAATCT CCTCTGGGGC GCCAAATTTC |
| | 3′ UTR |
| | --------------------- |
| | TGCACGGCCC AGCCTGGCTG |
| | ACGTGCCGGG TCGGACCGAC |
| | 3′ UTR |
| | ---------------------------------------------------------------------------------------- |
| 12301 | AAGCTGTAGG TCAGGGGAAG GACTAGAGGT TAGTGGAGAC CCCGTGCCAC AAAACACCAC AACAAAACAG CATATTGACA |
| | TTCGACATCC AGTCCCCTTC CTGATCTCCA ATCACCTCTG GGGCACGGTG TTTTGTGGTG TTGTTTTGTC GTATAACTGT |
| | 3′ UTR |
| | --------------------- |
| | CCTGGGATAG ACTAGGAGAT |
| | GGACCCTATC TGATCCTCTA |
| | 3′ UTR |
| | ---------------------------------------------------------------------------------- |
| 12401 | CTTCTGCTCT GCACAACCAG CCACACGGCA CAGTGCGCCG ACAATGGTGG CTGGTGGTGC GAGAACACAG GATCT |
| | GAAGACGAGA CGTGTTGGTC GGTGTGCCGT GTCACGCGGC TGTTACCACC GACCACCACG CTCTTGTGTC CTAGA |

RepliVax WN - Anchorless F inserted in place of ΔC-prM-E.
F insert starts at nucleotide position 229 bp and ends at 1806 bp.

| | |
|---|---|
| | 5′ UTR |
| | ---------------------------------------------------------------------------------------- |
| 1 | AGTAGTTCGC CTGTGTGAGC TGACAAACTT AGTAGTGTTT GTGAGGATTA ACAACAATTA ACACAGTGCG AGCTGTTTCT |
| | TCATCAAGCG GACACACTCG ACTGTTTGAA TCATCACAAA CACTCCTAAT TGTTGTTAAT TGTGTCACGC TCGACAAAGA |
| | C |
| | ---- |
| | 5′ UTR |
| | ----------------- |
| | M S • |
| | TAGCACGAAG ATCTCGATGT |
| | ATCGTGCTTC TAGAGCTACA |
| | C |
| | ---------------------------------------------------------------------------------------- |
| | • K K P G G P G K S R A V Y L L K R G M P R V L S L I |
| 101 | CTAAGAAACC AGGAGGGCCC GGCAAGAGCC GGGCTGTCTA TTTGCTAAAA CGCGGAATGC CCCGCGTGTT GTCCTTGATT |
| | GATTCTTTGG TCCTCCCGGG CCGTTCTCGG CCCGACAGAT AAACGATTTT GCGCCTTACG GGGCGCACAA CAGGAACTAA |
| | NS3 cleavage |
| | --------------- |
| | C |
| | ------ |
| | G L K Q K K R • |
| | GGACTTAAGC AAAAGAAGCG |
| | CCTGAATTCG TTTTCTTCGC |
| | NS3 cleavage Anchorless RSV F |
| | - ---------------------------------------------------------- |
| | partial C signal |
| | ----------------------------- |
| | • G G K T G I A V I M E L P I I K A N A I T T I L I A V |
| 201 | AGGGGGCAAG ACTGGTATAG CTGTGATCAT GGAACTGCCC ATCATCAAGG CCAACGCCAT CACCACCATC CTGATCGCCG |
| | TCCCCCGTTC TGACCATATC GACACTAGTA CCTTGACGGG TAGTAGTTCC GGTTGCGGTA GTGGTGGTAG GACTAGCGGC |
| | Anchorless RSV F |
| | --------------------- |
| | T F C F A S |
| | TGACCTTCTG CTTCGCCAGC |
| | ACTGGAAGAC GAAGCGGTCG |
| | Anchorless RSV F |
| | ---------------------------------------------------------------------------------------- |
| | S Q N I T E E F Y Q S T C S A V S K G Y L S A L R T G |
| 301 | AGCCAGAACA TCACCGAGGA ATTCTACCAG AGCACCTGCA GCGCCGTGAG CAAGGGCTAC CTGAGCGCCC TGCGGACCGG |
| | TCGGTCTTGT AGTGGCTCCT TAAGATGGTC TCGTGGACGT CGCGGCACTC GTTCCCGATG GACTCGCGGG ACGCCTGGCC |
| | Anchorless RSV F |
| | --------------------- |
| | W Y T S V I T • |
| | CTGGTACACC AGCGTGATCA |
| | GACCATGTGG TCGCACTAGT |
| | Anchorless RSV F |
| | ---------------------------------------------------------------------------------------- |
| | • I E L S N I K E N K C N G T D A K V K L I K Q E L D |
| 401 | CCATCGAGCT GTCCAACATC AAAGAAAACA AGTGCAACGG CACCGACGCC AAGGTGAAAC TGATCAAGCA GGAACTGGAC |
| | GGTAGCTCGA CAGGTTGTAG TTTCTTTTGT TCACGTTGCC GTGGCTGCGG TTCCACTTTG ACTAGTTCGT CCTTGACCTG |
| | Anchorless RSV F |
| | --------------------- |
| | K Y K N A V T • |
| | AAGTACAAGA ACGCCGTGAC |
| | TTCATGTTCT TGCGGCACTG |
| | Anchorless RSV F |
| | ---------------------------------------------------------------------------------------- |
| | • E L Q L L M Q S T P A A N N A A R R E L P R F M N Y T |
| 501 | CGAGCTGCAG CTGCTGATGC AGAGCACCCC TGCCGCCAAC AACCGGGCCA GACGCGAGCT GCCCCGGTTC ATGAACTACA |
| | GCTCGACGTC GACGACTACG TCTCGTGGGG ACGGCGGTTG TTGGCCCGGT CTGCGCTCGA CGGGGCCAAG TACTTGATGT |
| | Anchorless RSV F |
| | --------------------- |
| | L N N A K K |
| | CCCTGAACAA CGCCAAGAAA |
| | GGGACTTGTT GCGGTTCTTT |
| | Anchorless RSV F |
| | ---------------------------------------------------------------------------------------- |
| | T N V T L S K K R K R R F L G F L L G V G S A I A S G |
| 601 | ACCAACGTGA CCCTGAGCAA GAAGCGGAAG CGGCGGTTCC TGGGCTTCCT GCTGGGCGTG GGCAGCGCCA TCGCCAGCGG |
| | TGGTTGCACT GGGACTCGTT CTTCGCCTTC GCCGCCAAGG ACCCGAAGGA CGACCCGCAC CCGTCGCGGT AGCGGTCGCC |
| | Anchorless RSV F |
| | --------------------- |
| | I A V S K V L • |
| | CATCGCCGTG TCCAAGGTGC |
| | GTAGCGGCAC AGGTTCCACG |
| | Anchorless RSV F |
| | ---------------------------------------------------------------------------------------- |
| | • H L E G E V N K I K S A L L S T N K A V V S L S N G |
| 701 | TGCACCTGGA AGGCGAGGTG AACAAGATCA AGTCCGCCCT GCTGTCCACC AACAAGGCCG TGGTGTCCCT GAGCAACGGC |
| | ACGTGGACCT TCCGCTCCAC TTGTTCTAGT TCAGGCGGGA CGACAGGTGG TTGTTCCGGC ACCACAGGGA CTCGTTGCCG |
| | Anchorless RSV F |
| | --------------------- |
| | V S V L T S K • |
| | GTGAGCGTGC TGACCAGCAA |
| | CACTCGCACG ACTGGTCGTT |
| | Anchorless RSV F |
| | ---------------------------------------------------------------------------------------- |
| | • V L D L K N Y I D K Q L L P I V N K Q S C S I S N I E |
| 801 | GGTGCTGGAT CTGAAGAACT ACATCGACAA GCAGCTGCTG CCCATCGTGA ACAAGCAGAG CTGCAGCATC AGCAACATCG |
| | CCACGACCTA GACTTCTTGA TGTAGCTGTT CGTCGACGAC GGGTAGCACT TGTTCGTCTC GACGTCGTAG TCGTTGTAGC |
| | Anchorless RSV F |
| | --------------------- |
| | T V I E F Q |
| | AGACCGTGAT CGAGTTCCAG |
| | TCTGGCACTA GCTCAAGGTC |
| | Anchorless RSV F |
| | ---------------------------------------------------------------------------------------- |
| | Q K N N R L L E I T R E F S V N A G V T T P V S T Y M |
| 901 | CAGAAGAACA ACCGGCTGCT GGAAATCACC CGGGAGTTCA GCGTGAACGC CGGCGTGACC ACCCCCGTGA GCACCTACAT |
| | GTCTTCTTGT TGGCCGACGA CCTTTAGTGG GCCCTCAAGT CGCACTTGCG GCCGCACTGG TGGGGGCACT CGTGGATGTA |
| | Anchorless RSV F |
| | --------------------- |
| | L T N S E L L • |
| | GCTGACCAAC AGCGAGCTGC |
| | CGACTGGTTG TCGCTCGACG |
| | Anchorless RSV F |
| | ---------------------------------------------------------------------------------------- |
| | • S L I N D M P I T N D Q K K L M S N N V Q I V R Q Q |
| 1001 | TGTCCCTGAT CAATGACATG CCCATCACCA ACGACCAGAA GAAACTGATG AGCAACAACG TGCAGATCGT GCGGCAGCAG |
| | ACAGGGACTA GTTACTGTAC GGGTAGTGGT TGCTGGTCTT CTTTGACTAC TCGTTGTTGC ACGTCTAGCA CGCCGTCGTC |
| | Anchorless RSV F |
| | --------------------- |
| | S Y S I M S I • |
| | AGCTACTCCA TCATGAGCAT |
| | TCGATGAGGT AGTACTCGTA |
| | Anchorless RSV F |
| | ---------------------------------------------------------------------------------------- |
| | • I K E E V L A Y V V Q L P L Y G V I D T P C W K L H T |
| 1101 | CATCAAAGAA GAGGTGCTGG CCTACGTGGT GCAGCTGCCC CTGTACGGCG TGATCGACAC CCCCTGCTGG AAGCTGCACA |
| | GTAGTTTCTT CTCCACGACC GGATGCACCA CGTCGACGGG GACATGCCGC ACTAGCTGTG GGGGACGACC TTCGACGTGT |
| | Anchorless RSV F |
| | --------------------- |
| | S P L C T T |
| | CCAGCCCCCT GTGCACCACC |
| | GGTCGGGGGA CACGTGGTGG |
| | Anchorless RSV F |
| | ---------------------------------------------------------------------------------------- |
| | N T K E G S N I C L T R T D R G W Y C N N A G S V S F |
| 1201 | AACACCAAAG AGGGCAGCAA CATCTGCCTG ACCCGGACCG ACCGGGGCTG GTACTGCAAC AACGCCGGCA GCGTGAGCTT |
| | TTGTGGTTTC TCCCGTCGTT GTAGACGGAC TGGGCCTGGC TGGCCCCGAC CATGACGTTG TTGCGGCCGT CGCACTCGAA |
| | Anchorless RSV F |
| | --------------------- |
| | F P L A D T C • |
| | CTTCCCCCTG GCCGACACCT |
| | GAAGGGGGAC CGGCTGTGGA |
| | Anchorless RSV F |
| | ---------------------------------------------------------------------------------------- |
| | • K V Q S N R V F C D T M N S L T L P S E V N L C N I |
| 1301 | GCAAGGTGCA GAGCAACCGG GTGTTCTGCG ACACCATGAA CAGCCTGACC CTGCCCTCCG AGGTGAACCT GTGCAACATC |
| | CGTTCCACGT CTCGTTGGCC CACAAGACGC TGTGGTACTT GTCGGACTGG GACGGGAGGC TCCACTTGGA CACGTTGTAG |
| | Anchorless RSV F |
| | --------------------- |
| | D I F N P K Y • |
| | GACATCTTCA ACCCCAAGTA |
| | CTGTAGAAGT TGGGGTTCAT |
| | Anchorless RSV F |
| | ---------------------------------------------------------------------------------------- |
| | • D C K I M T S K T D V S S S V I T S L G A I V S C Y G |
| 1401 | CGACTGCAAG ATCATGACCT CCAAGACCGA CGTGAGCAGC TCCGTGATCA CCTCCCTGGG CGCCATCGTG AGCTGCTACG |
| | GCTGACGTTC TAGTACTGGA GGTTCTGGCT GCACTCGTCG AGGCACTAGT GGAGGGACCC GCGGTAGCAC TCGACGATGC |
| | Anchorless RSV F |
| | --------------------- |
| | K T K C T A |
| | GCAAGACCAA GTGCACCGCC |
| | CGTTCTGGTT CACGTGGCGG |
| | Anchorless RSV F |
| | ---------------------------------------------------------------------------------------- |
| | S N K N R G I I K T F S N G C D Y V S N K G V D T V S |
| 1501 | AGCAACAAGA ACCGGGGCAT CATCAAGACC TTCAGCAACG GCTGCGACTA CGTGAGCAAC AAGGGCGTGG ACACCGTGAG |
| | TCGTTGTTCT TGGCCCCGTA GTAGTTCTGG AAGTCGTTGC CGACGCTGAT GCACTCGTTG TTCCCGCACC TGTGGCACTC |
| | Anchorless RSV F |
| | --------------------- |
| | V G N T L Y Y • |
| | CGTGGGCAAC ACACTGTACT |
| | GCACCCGTTG TGTGACATGA |
| | Anchorless RSV F |
| | ---------------------------------------------------------------------------------------- |
| | • V N K Q E G K S L Y V K G E P I I N F Y D P L V F P |
| 1601 | ACGTGAATAA GCAGGAAGGC AAGAGCCTGT ACGTGAAGGG CGAGCCTATC ATCAACTTCT ACGACCCCCT GGTGTTCCCC |
| | TGCACTTATT CGTCCTTCCG TTCTCGGACA TGCACTTCCC GCTCGGATAG TAGTTGAAGA TGCTGGGGGA CCACAAGGGG |
| | Anchorless RSV F |
| | --------------------- |
| | S D E F D A S • |
| | AGCGACGAGT TCGACGCCAG |
| | TCGCTGCTCA AGCTGCGGTC |
| | Anchorless RSV F |
| | ---------------------------------------------------------------------------------------- |
| | • I S Q V N E K I N Q S L A F I R K S D E L L H N V N A |
| 1701 | CATCAGCCAG GTGAACGAGA AGATCAACCA GAGCCTGGCC TTCATCCGGA AGAGCGACGA GCTGCTGCAC AATGTGAATG |
| | GTAGTCGGTC CACTTGCTCT TCTAGTTGGT CTCGGACCGG AAGTAGGCCT TCTCGCTGCT CGACGACGTG TTACACTTAC |
| | Anchorless RSV F |
| | --------------------- |
| | G K S T T N |
| | CCGGCAAGAG CACCACCAAT |
| | GGCCGTTCTC GTGGTGGTTA |
| | Anchorless RSV F pre E/NS1 signal |
| | ------ ---------- |
| | Transmembrane |
| | domain of |
| | FMDV 2A WNV E (split) |
| | -------------------------------------------------------- ---------------- |
| | I M N F D L L K L A G D V E S N P G P A R D R S I A L |
| 1801 | ATCATGAATT TTGATCTGCT CAAACTTGCA GGCGATGTAG AATCAAATCC TGGACCCGCC CGGGACAGGT CCATAGCTCT |
| | TAGTACTTAA AACTAGACGA GTTTGAACGT CCGCTACATC TTAGTTTAGG ACCTGGGCGG GCCCTGTCCA GGTATCGAGA |
| | Transmembrane domain |
| | of WNV E (split) |
| | --------------------- |
| | T F L A V G G • |
| | CACGTTTCTC GCAGTTGGAG |
| | GTGCAAAGAG CGTCAACCTC |
| | Transmembrane domain of WNV E (split) |
| | -------------------------------------- |
| | NS1 |
| | -------------------------------------------------- |
| | • V L L F L S V N V H A D T G C A I D I S R Q E L R C |
| 1901 | GAGTTCTGCT CTTCCTCTCC GTGAACGTGC ACGCTGACAC TGGGTGTGCC ATAGACATCA GCCGGCAAGA GCTGAGATGT |
| | CTCAAGACGA GAAGGAGAGG CACTTGCACG TGCGACTGTG ACCCACACGG TATCTGTAGT CGGCCGTTCT CGACTCTACA |
| | NS1 |
| | --------------------- |
| | G S G V F I H • |
| | GGAAGTGGAG TGTTCATACA |
| | CCTTCACCTC ACAAGTATGT |
| | NS1 |
| | ---------------------------------------------------------------------------------------- |
| | • N D V E A W M D R Y K Y Y P E T P Q G L A K I I Q |
| 2001 | CAATGATGTG GAGGCTTGGA TGGACCGGTA CAAGTATTAC CCTGAAACGC CACAAGGCCT AGCCAAGATC ATTCAGAAAG |
| | GTTACTACAC CTCCGAACCT ACCTGGCCAT GTTCATAATG GGACTTTGCG GTGTTCCGGA TCGGTTCTAG TAAGTCTTTC |
| | NS1 |
| | --------------------- |
| | H K E G V C |
| | CTCATAAGGA AGGAGTGTGC |
| | GAGTATTCCT TCCTCACACG |
| | NS1 |
| | ---------------------------------------------------------------------------------------- |
| | G L R S V S R L E H Q M W E A V K D E L N T L L K E N |
| 2101 | GGTCTACGAT CAGTTTCCAG ACTGGAGCAT CAAATGTGGG AAGCAGTGAA GGACGAGCTG AACACTCTTT TGAAGGAGAA |
| | CCAGATGCTA GTCAAAGGTC TGACCTCGTA GTTTACACCC TTCGTCACTT CCTGCTCGAC TTGTGAGAAA ACTTCCTCTT |
| | NS1 |
| | --------------------- |
| | G V D L S V V • |
| | TGGTGTGGAC CTTAGTGTCG |
| | ACCACACCTG GAATCACAGC |
| | NS1 |
| | ---------------------------------------------------------------------------------------- |
| | • V E K Q G G M Y K S A P K R L T A T T E K L E I G W |
| 2201 | TGGTTGAGAA ACAAGGGGGA ATGTACAAGT CAGCACCTAA ACGCCTCACC GCCACCACGG AAAAATTGGA AATTGGCTGG |
| | ACCAACTCTT TGTTCCCCCT TACATGTTCA GTCGTGGATT TGCGGAGTGG CGGTGGTGCC TTTTTAACCT TTAACCGACC |
| | NS1 |
| | --------------------- |
| | K A W G K S I • |
| | AAGGCCTGGG GAAAGAGTAT |
| | TTCCGGACCC CTTTCTCATA |
| | NS1 |
| | ---------------------------------------------------------------------------------------- |
| | • L F A P E L A N N T F V V D G P E T K E C P T Q N R A |
| 2301 | TTTGTTTGCA CCAGAACTCG CCAACAACAC CTTTGTGGTT GATGGTCCGG AGACCAAGGA ATGTCCGACT CAGAATCGCG |
| | AAACAAACGT GGTCTTGAGC GGTTGTTGTG GAAACACCAA CTACCAGGCC TCTGGTTCCT TACAGGCTGA GTCTTAGCGC |
| | NS1 |
| | --------------------- |
| | W N S L E V |
| | CTTGGAATAG CTTAGAAGTG |
| | GAACCTTATC GAATCTTCAC |
| | NS1 |
| | ---------------------------------------------------------------------------------------- |
| | E D F G F G L T S T R M F L K V R E S N T T E C D S K |
| 2401 | GAGGATTTTG GATTTGGTCT CACCAGCACT CGGATGTTCC TGAAGGTCAG AGAGAGCAAC ACAACTGAAT GTGACTCGAA |
| | CTCCTAAAAC CTAAACCAGA GTGGTCGTGA GCCTACAAGG ACTTCCAGTC TCTCTCGTTG TGTTGACTTA CACTGAGCTT |
| | NS1 |
| | --------------------- |
| | I I G T A V K • |
| | GATCATTGGA ACGGCTGTCA |
| | CTAGTAACCT TGCCGACAGT |
| | NS1 |
| | ---------------------------------------------------------------------------------------- |
| | • N N L A I H S D L S Y W I E S R L N D T W K L E R A |
| 2501 | AGAACAACTT GGCGATCCAC AGTGACCTGT CCTATTGGAT TGAAAGCAGG CTCAATGATA CGTGGAAGCT TGAAAGGGCA |
| | TCTTGTTGAA CCGCTAGGTG TCACTGGACA GGATAACCTA ACTTTCCTCC GAGTTACTAT GCACCTTCGA ACTTTCCCGT |
| | NS1 |
| | --------------------- |
| | V L G E V K S • |
| | GTTCTGGGTG AAGTCAAATC |
| | CAAGACCCAC TTCAGTTTAG |
| | NS1 |
| | ---------------------------------------------------------------------------------------- |
| | • C T W P E T H T L W G D G I L E S D L I I P V T L A G |
| 2601 | ATGTACGTGG CCTGAGACGC ATACCTTGTG GGGCGATGGA ATCCTTGAGA GTGACTTGAT AATACCAGTC ACACTGGCGG |
| | TACATGCACC GGACTCTGCG TATGGAACAC CCCGCTACCT TAGGAACTCT CACTGAACTA TTATGGTCAG TGTGACCGCC |
| | NS1 |
| | --------------------- |
| | P R S N H N |
| | GACCACGAAG CAATCACAAT |
| | CTGGTGCTTC GTTAGTGTTA |
| | NS1 |
| | ---------------------------------------------------------------------------------------- |
| | R R P G Y K T Q N Q G P W D E G R V E I D F D Y C P G |
| 2701 | CGGAGACCTG GGTATAAGAC ACAAAACCAG GGCCCATGGG ACGAAGGCCG GGTAGAGATT GACTTCGATT ACTGCCCAGG |
| | GCCTCTGGAC CCATATTCTG TGTTTTGGTC CCGGGTACCC TGCTTCCGGC CCATCTCTAA CTGAAGCTAA TGACGGGTCC |
| | NS1 |
| | --------------------- |
| | T T V T L S E • |
| | AACTACGGTC ACCCTGAGTG |
| | TTGATGCCAG TGGGACTCAC |
| | NS1 |
| | ---------------------------------------------------------------------------------------- |
| | • S C G H R G P A T R T T T E S G K L I T D W C C R S |
| 2801 | AGAGCTGCGG ACACCGTGGA CCTGCCACTC GCACCACCAC AGAGAGCGGA AAGTTGATAA CAGATTGGTG CTGCAGGAGC |
| | TCTCGACGCC TGTGGCACCT GGACGGTGAG CGTGGTGGTG TCTCTCGCCT TTCAACTATT GTCTAACCAC GACGTCCTCG |
| | NS1 |
| | --------------------- |
| | C T L P P L R • |
| | TGCACCTTAC CACCACTGCG |
| | ACGTGGAATG GTGGTGACGC |
| | NS1 |
| | ---------------------------------------------------------------------------------------- |
| | • Y Q T D S G C W Y G M E I R P Q R H D E K T L V Q S Q |
| 2901 | CTACCAAACT GACAGCGGCT GTTGGTATGG TATGGAGATC AGACCACAGA GACATGATGA AAAGACCCTC GTGCAGTCAC |
| | GATGGTTTGA CTGTCGCCGA CAACCATACC ATACCTCTAG TCTGGTGTCT CTGTACTACT TTTCTGGGAG CACGTCAGTG |
| | NS2A |
| | --------- |
| | NS1 |
| | ------------ |
| | V N A Y N A |
| | AAGTGAATGC TTATAATGCT |
| | TTCACTTACG AATATTACGA |
| | NS2A |
| | ---------------------------------------------------------------------------------------- |
| | D M I D P F Q L G L L V V F L A T Q E V L R K R W T A |
| 3001 | GATATGATTG ACCCTTTTCA GTTGGGCCTT CTGGTCGTGT TCTTGGCCAC CCAGGAGGTC CTTCGCAAGA GGTGGACAGC |
| | CTATACTAAC TGGGAAAAGT CAACCCGGAA GACCAGCACA AGAACCGGTG GGTCCTCCAG GAAGCGTTCT CCACCTGTCG |
| | NS2A |
| | --------------------- |
| | K I S M P A I • |
| | CAAGATCAGC ATGCCAGCTA |
| | GTTCTAGTCG TACGGTCGAT |
| | NS2A |
| | ---------------------------------------------------------------------------------------- |
| | • L I A L L V L V F G G I T Y T D V L R Y V I L V G A |
| 3101 | TACTGATTGC TCTGCTAGTC CTGGTGTTTG GGGGCATTAC TTACACTGAT GTGTTACGCT ATGTCATCTT GGTGGGGGCA |
| | ATGACTAACG AGACGATCAG GACCACAAAC CCCCGTAATG AATGTGACTA CACAATGCGA TACAGTAGAA CCACCCCCGT |
| | NS2A |
| | --------------------- |
| | A F A E S N S • |
| | GCTTTCGCAG AATCTAATTC |
| | CGAAAGCGTC TTAGATTAAG |
| | NS2A |
| | ---------------------------------------------------------------------------------------- |
| | • G G D V V H L A L M A T F K I Q P V F M V A S F L K A |
| 3201 | GGGAGGAGAC GTGGTACACT TGGCGCTCAT GGCGACCTTC AAGATACAAC CAGTGTTTAT GGTGGCATCG TTTCTTAAAG |
| | CCCTCCTCTG CACCATGTGA ACCGCGAGTA CCGCTGGAAG TTCTATGTTG GTCACAAATA CCACCGTAGC AAAGAATTTC |
| | NS2A |
| | --------------------- |
| | R W T N Q E |
| | CGAGATGGAC CAACCAGGAG |
| | GCTCTACCTG GTTGGTCCTC |
| | NS2A |
| | ---------------------------------------------------------------------------------------- |
| | N I L L M L A A V F F Q M A Y H D A R Q I L L W E I P |
| 3301 | AACATTTTGT TGATGTTGGC GGCTGTTTTC TTTCAAATGG CTTATCACGA TGCCCGCCAA ATTCTGCTCT GGGAGATCCC |
| | TTGTAAAACA ACTACAACCG CCGACAAAAG AAAGTTTACC GAATAGTGCT ACGGGCGGTT TAAGACGAGA CCCTCTAGGG |
| | NS2A |
| | --------------------- |
| | D V L N S L A • |
| | TGATGTGTTG AATTCACTGG |
| | ACTACACAAC TTAAGTGACC |
| | NS2A |
| | ---------------------------------------------------------------------------------------- |
| | • I A W M I L R A I T F T T T S N V V V P L L A L L T |
| 3401 | CAATAGCTTG GATGATACTG AGAGCCATAA CATTCACAAC GACATCAAAC GTGGTTGTTC CGCTGCTAGC CCTGCTAACA |
| | GTTATCGAAC CTACTATGAC TCTCGGTATT GTAAGTGTTG CTGTAGTTTG CACCAACAAG GCGACGATCG GGACGATTGT |
| | NS2A |
| | --------------------- |
| | P G L R C L N • |
| | CCCGGGCTGA GATGCTTGAA |
| | GGGCCCGACT CTACGAACTT |
| | NS2A |
| | ---------------------------------------------------------------------------------------- |
| | • L D V Y R I L L L M V G I G S L I R E K R S A A A K K |
| 3501 | TCTGGATGTG TACAGGATAC TGCTGTTGAT GGTCGGAATA GGCAGCTTGA TCAGGGAGAA GAGGAGCGCA GCTGCAAAAA |
| | AGACCTACAC ATGTCCTATG ACGACAACTA CCAGCCTTAT CCGTCGAACT AGTCCCTCTT CTCCTCGCGT CGACGTTTTT |
| | NS2A |
| | --------------------- |
| | K G A S L L |
| | AGAAAGGAGC AAGTCTGCTA |
| | TCTTTCCTCG TTCAGACGAT |
| | NS2A |
| | ---------------------------------------------------------------------------------------- |
| | C L A L A S T G L F N P M I L A A G L I A C D P N R K |
| 3601 | TGCTTGGCTC TAGCCTCAAC AGGACTCTTC AACCCCATGA TCCTTGCTGC TGGACTGATT GCATGTGATC CCAACCGTAA |
| | ACGAACCGAG ATCGGAGTTG TCCTGAGAAG TTGGGGTACT AGGAACGACG ACCTGACTAA CGTACACTAG GGTTGGCATT |
| | NS2B |
| | ------------------ |
| | NS2A |
| | ---- |
| | R G W P A T E • |
| | ACGCGGGTGG CCCGCAACTG |
| | TGCGCCCACC GGGCGTTGAC |
| | NS2B |
| | ---------------------------------------------------------------------------------------- |
| | • V M T A V G L M F A I V G G L A E L D I D S M A I P |
| 3701 | AAGTGATGAC AGCTGTCGGC CTAATGTTTG CCATCGTCGG AGGGCTGGCA GAGCTTGACA TTGACTCCAT GGCCATTCCA |
| | TTCACTACTG TCGACAGCCG GATTACAAAC GGTAGCAGCC TCCCGACCGT CTCGAACTGT AACTGAGGTA CCGGTAAGGT |
| | NS2B |
| | --------------------- |
| | M T I A G L M • |
| | ATGACTATCG CGGGGCTCAT |
| | TACTGATAGC GCCCCGAGTA |
| | NS2B |
| | ---------------------------------------------------------------------------------------- |
| | • F A A F V I S G K S T D M W I E R T A D I S W E S D A |
| 3801 | GTTTGCTGCT TTCGTGATTT CTGGGAAATC AACAGATATG TGGATTGAGA GAACGGCGGA CATTTCCTGG GAAAGTGATG |
| | CAAACGACGA AAGCACTAAA GACCCTTTAG TTGTCTATAC ACCTAACTCT CTTGCCGCCT GTAAAGGACC CTTTCACTAC |
| | NS2B |
| | --------------------- |
| | E I T G S S |
| | CAGAGATTAC AGGCTCGAGC |
| | GTCTCTAATG TCCGAGCTCG |
| | NS2B |
| | ---------------------------------------------------------------------------------------- |
| | E R V D V R L D D D G N F Q L M N D P G A P W K I W M |
| 3901 | GAAAGAGTTG ATGTGCGGCT TGATGATGAT GGAAACTTCC AGCTCATGAA TGATCCAGGA GCACCTTGGA AGATATGGAT |
| | CTTTCTCAAC TACACGCCGA ACTACTACTA CCTTTGAAGG TCGAGTACTT ACTAGGTCCT CGTGGAACCT TCTATACCTA |
| | NS2B |
| | --------------------- |
| | L R M V C L A • |
| | GCTCAGAATG GTCTGTCTCG |
| | CGAGTCTTAC CAGACAGAGC |
| | NS3 |
| | ---- |
| | NS2B |
| | ------------------------------------------------------------------------------------ |
| | • I S A Y T P W A I L P S V V G F W I T L Q Y T K R G |
| 4001 | CGATTAGTGC GTACACCCCC TGGGCAATCT TGCCCTCAGT AGTTGGATTT TGGATAACTC TCCAATACAC AAAGAGAGGA |
| | GCTAATCACG CATGTGGGGG ACCCGTTAGA ACGGGAGTCA TCAACCTAAA ACCTATTGAG AGGTTATGTG TTTCTCTCCT |
| | NS3 |
| | --------------------- |
| | G V L W D T P • |
| | GGCGTGTTGT GGGACACTCC |
| | CCGCACAACA CCCTGTGAGG |
| | NS3 |
| | ---------------------------------------------------------------------------------------- |
| | • S P K E Y K K G D T T T G V Y R I M T R G L L G S Y Q |
| 4101 | CTCACCAAAG GAGTACAAAA AGGGGGACAC GACCACCGGC GTCTACAGGA TCATGACTCG TGGGCTGCTC GGCAGTTATC |
| | GAGTGGTTTC CTCATGTTTT TCCCCCTGTG CTGGTGGCCG CAGATGTCCT AGTACTGAGC ACCCGACGAG CCGTCAATAG |
| | NS3 |
| | --------------------- |
| | A G A G V M |
| | AAGCAGGAGC AGGCGTGATG |
| | TTCGTCCTCG TCCGCACTAC |
| | NS3 |
| | ---------------------------------------------------------------------------------------- |
| | V E G V F H T L W H T T K G A A L M S G E G R L D P Y |
| 4201 | GTTGAAGGTG TTTTCCACAC CCTTTGGCAT ACAACAAAAG GAGCCGCTTT GATGAGCGGA GAGGGCCGCC TGGACCCATA |
| | CAACTTCCAC AAAAGGTGTG GGAAACCGTA TGTTGTTTTC CTCGGCGAAA CTACTCGCCT CTCCCGGCGG ACCTGGGTAT |
| | NS3 |
| | --------------------- |
| | W G S V K E D • |
| | CTGGGGCAGT GTCAAGGAGG |
| | GACCCCGTCA CAGTTCCTCC |
| | NS3 |
| | ---------------------------------------------------------------------------------------- |
| | • R L C Y G G P W K L Q H K W N G Q D E V Q M I V V E |
| 4301 | ATCGACTTTG TTACGGAGGA CCCTGGAAAT TGCAGCACAA GTGGAACGGG CAGGATGAGG TGCAGATGAT TGTGGTGGAA |
| | TAGCTGAAAC AATGCCTCCT GGGACCTTTA ACGTCGTGTT CACCTTGCCC GTCCTACTCC ACGTCTACTA ACACCACCTT |
| | NS3 |
| | --------------------- |
| | P G K N V K N • |
| | CCTGGCAAGA ACGTTAAGAA |
| | GGACCGTTCT TGCAATTCTT |
| | NS3 |
| | ---------------------------------------------------------------------------------------- |
| | • V Q T K P G V F K T P E G E I G A V T L D F P T G T S |
| 4401 | CGTCCAGACG AAACCAGGGG TGTTCAAAAC ACCTGAAGGA GAAATCGGGG CCGTGACTTT GGACTTCCCC ACTGGAACAT |
| | GCAGGTCTGC TTTGGTCCCC ACAAGTTTTG TGGACTTCCT CTTTAGCCCC GGCACTGAAA CCTGAAGGGG TGACCTTGTA |
| | NS3 |
| | --------------------- |
| | G S P I V D |
| | CAGGCTCACC AATAGTGGAC |
| | GTCCGAGTGG TTATCACCTG |
| | NS3 |
| | ---------------------------------------------------------------------------------------- |
| | K N G D V I G L Y G N G V I M P N G S Y I S A I V Q G |
| 4501 | AAAAACGGTG ATGTGATTGG GCTTTATGGC AATGGAGTCA TAATGCCCAA CGGCTCATAC ATAAGCGCGA TAGTGCAGGG |
| | TTTTTGCCAC TACACTAACC CGAAATACCG TTACCTCAGT ATTACGGGTT GCCGAGTATG TATTCGCGCT ATCACGTCCC |
| | NS3 |
| | --------------------- |
| | E R M D E P I • |
| | TGAAAGGATG GATGAGCCAA |
| | ACTTTCCTAC CTACTCGGTT |
| | NS3 |
| | ---------------------------------------------------------------------------------------- |
| | • P A G F E P E M L R K K Q I T V L D L H P G A G K T |
| 4601 | TCCCAGCCGG ATTCGAACCT GAGATGCTGA GGAAAAAACA GATCACTGTA CTGGATCTCC ATCCCGGCGC CGGTAAAACA |
| | AGGGTCGGCC TAAGCTTGGA CTCTACGACT CCTTTTTTGT CTAGTGACAT GACCTAGAGG TAGGGCCGCG GCCATTTTGT |
| | NS3 |
| | --------------------- |
| | R R I L P Q I • |
| | AGGAGGATTC TGCCACAGAT |
| | TCCTCCTAAG ACGGTGTCTA |
| | NS3 |
| | ---------------------------------------------------------------------------------------- |
| | • I K E A I N R R L R T A V L A P T R V V A A E M A E A |
| 4701 | CATCAAAGAG GCCATAAACA GAAGACTGAG AACAGCCGTG CTAGCACCAA CCAGGGTTGT GGCTGCTGAG ATGGCTGAAG |
| | GTAGTTTCTC CGGTATTTGT CTTCTGACTC TTGTCGGCAC GATCGTGGTT GGTCCCAACA CCGACGACTC TACCGACTTC |
| | NS3 |
| | --------------------- |
| | L R G L P I |
| | CACTGAGAGG ACTGCCCATC |
| | GTGACTCTCC TGACGGGTAG |
| | NS3 |
| | ---------------------------------------------------------------------------------------- |
| | R Y Q T S A V P R E H N G N E I V D V M C H A T L T H |
| 4801 | CGGTACCAGA CATCCGCAGT GCCCAGAGAA CATAATGGAA ATGAGATTGT TGATGTCATG TGTCATGCTA CCCTCACCCA |
| | GCCATGGTCT GTAGGCGTCA CGGGTCTCTT GTATTACCTT TACTCTAACA ACTACAGTAC ACAGTACGAT GGGAGTGGGT |
| | NS3 |
| | --------------------- |
| | R L M S P H R • |
| | CAGGCTGATG TCTCCTCACA |
| | GTCCGACTAC AGAGGAGTGT |
| | NS3 |
| | ---------------------------------------------------------------------------------------- |
| | • V P N Y N L F V M D E A H F T D P A S I A A R G Y I |
| 4901 | GGGTGCCGAA CTACAACCTG TTCGTGATGG ATGAGGCTCA TTTCACCGAC CCAGCTAGCA TTGCAGCAAG AGGTTACATT |
| | CCCACGGCTT GATGTTGGAC AAGCACTACC TACTCCGAGT AAAGTGGCTG GGTCGATCGT AACGTCGTTC TCCAATGTAA |
| | NS3 |
| | --------------------- |
| | S T K V E L G • |
| | TCCACAAAGG TCGAGCTAGG |
| | AGGTGTTTCC AGCTCGATCC |
| | NS3 |
| | ---------------------------------------------------------------------------------------- |
| | • E A A A I F M T A T P P G T S D P F P E S N S P I S D |
| 5001 | GGAGGCGGCG GCAATATTCA TGACAGCCAC CCCACCAGGC ACTTCAGATC CATTCCCAGA GTCCAATTCA CCAATTTCCG |
| | CCTCCGCCGC CGTTATAAGT ACTGTCGGTG GGGTGGTCCG TGAAGTCTAG GTAAGGGTCT CAGGTTAAGT GGTTAAAGGC |
| | NS3 |
| | --------------------- |
| | L Q T E I P |
| | ACTTACAGAC TGAGATCCCG |
| | TGAATGTCTG ACTCTAGGGC |
| | NS3 |
| | ---------------------------------------------------------------------------------------- |
| | D R A W N S G Y E W I T E Y T G K T V W F V P S V K M |
| 5101 | GATCGAGCTT GGAACTCTGG ATACGAATGG ATCACAGAAT ACACCGGGAA GACGGTTTGG TTTGTGCCTA GTGTTAAGAT |
| | CTAGCTCGAA CCTTGAGACC TATGCTTACC TAGTGTCTTA TGTGGCCCTT CTGCCAAACC AAACACGGAT CACAATTCTA |
| | NS3 |
| | --------------------- |
| | G N E I A L C • |
| | GGGGAATGAG ATTGCCCTTT |
| | CCCCTTACTC TAACGGGAAA |
| | NS3 |
| | ---------------------------------------------------------------------------------------- |
| | • L Q R A G K K V V Q L N R K S Y E T E Y P K C K N D |
| 5201 | GCCTACAACG TGCTGGAAAG AAAGTAGTCC AATTGAACAG AAAGTCGTAC GAGACGGAGT ACCCAAAATG TAAGAACGAT |
| | CGGATGTTGC ACGACCTTTC TTTCATCAGG TTAACTTGTC TTTCAGCATG CTCTGCCTCA TGGGTTTTAC ATTCTTGCTA |
| | NS3 |
| | --------------------- |
| | D W D F V I T • |
| | GATTGGGACT TTGTTATCAC |
| | CTAACCCTGA AACAATAGTG |
| | NS3 |
| | ---------------------------------------------------------------------------------------- |
| | • T D I S E M G A N F K A S R V I D S R K S V K P T I I |
| 5301 | AACAGACATA TCTGAAATGG GGGCTAACTT CAAGGCGAGC AGGGTGATTG ACAGCCGGAA GAGTGTGAAA CCAACCATCA |
| | TTGTCTGTAT AGACTTTACC CCCGATTGAA GTTCCGCTCG TCCCACTAAC TGTCGGCCTT CTCACACTTT GGTTGGTAGT |
| | NS3 |
| | --------------------- |
| | T E G E G R |
| | TAACAGAAGG AGAAGGGAGA |
| | ATTGTCTTCC TCTTCCCTCT |
| | NS3 |
| | ---------------------------------------------------------------------------------------- |
| | V I L G E P S A V T A A S A A Q R R G R I G R N P S Q |
| 5401 | GTGATCCTGG GAGAACCATC TGCAGTGACA GCAGCTAGTG CCGCCCAGAG ACGTGGACGT ATCGGTAGAA ATCCGTCGCA |
| | CACTAGGACC CTCTTGGTAG ACGTCACTGT CGTCGATCAC GGCGGGTCTC TGCACCTGCA TAGCCATCTT TAGGCAGCGT |
| | NS3 |
| | --------------------- |
| | V G D E Y C Y • |
| | AGTTGGTGAT GAGTACTGTT |
| | TCAACCACTA CTCATGACAA |
| | NS3 |
| | ---------------------------------------------------------------------------------------- |
| | • G G H T N E D D S N F A H W T E A R I M L D N I N M |
| 5501 | ATGGGGGGCA CACGAATGAA GACGACTCGA ACTTCGCCCA TTGGACTGAG GCACGAATCA TGCTGGACAA CATCAACATG |
| | TACCCCCCGT GTGCTTACTT CTGCTGAGCT TGAAGCGGGT AACCTGACTC CGTGCTTAGT ACGACCTGTT GTAGTTGTAC |
| | NS3 |
| | --------------------- |
| | P N G L I A Q • |
| | CCAAACGGAC TGATCGCTCA |
| | GGTTTGCCTG ACTAGCGAGT |
| | NS3 |
| | ---------------------------------------------------------------------------------------- |
| | • F Y Q P E R E K V Y T M D G E Y R L R G E E R K N F L |
| 5601 | ATTCTACCAA CCAGAGCGTG AGAAGGTATA TACCATGGAT GGGGAATACC GGCTCAGAGG AGAAGAGAGA AAAAACTTTC |
| | TAAGATGGTT GGTCTCGCAC TCTTCCATAT ATGGTACCTA CCCCTTATGG CCGAGTCTCC TCTTCTCTCT TTTTTGAAAG |
| | NS3 |
| | --------------------- |
| | E L L R T A |
| | TGGAACTGTT GAGGACTGCA |
| | ACCTTGACAA CTCCTGACGT |
| | NS3 |
| | ---------------------------------------------------------------------------------------- |
| | D L P V W L A Y K V A A A G V S Y H D R R W C F D G P |
| 5701 | GATCTGCCAG TTTGGCTGGC TTACAAGGTT GCAGCGGCTG GAGTGTCATA CCACGACCGG AGGTGGTGCT TTGATGGTCC |
| | CTAGACGGTC AAACCGACCG AATGTTCCAA CGTCGCCGAC CTCACAGTAT GGTGCTGGCC TCCACCACGA AACTACCAGG |
| | NS3 |
| | --------------------- |
| | R T N T I L E • |
| | TAGGACAAAC ACAATTTTAG |
| | ATCCTGTTTG TGTTAAAATC |
| | NS3 |
| | ---------------------------------------------------------------------------------------- |
| | • D N N E V E V I T K L G E R K I L R P R W I D A R V |
| 5801 | AAGACAACAA CGAAGTGGAA GTCATCACGA AGCTTGGTGA AAGGAAGATT CTGAGGCCGC GCTGGATTGA CGCCAGGGTG |
| | TTCTGTTGTT GCTTCACCTT CAGTAGTGCT TCGAACCACT TTCCTTCTAA GACTCCGGCG CGACCTAACT GCGGTCCCAC |
| | NS3 |
| | --------------------- |
| | Y S D H Q A L • |
| | TACTCGGATC ACCAGGCACT |
| | ATGAGCCTAG TGGTCCGTGA |
| | NS4A |
| | --------------------------------------------------- |
| | NS3 |
| | ------------------------------------- |
| | • K A F K D F A S G K R S Q I G L I E V L G K M P E H F |
| 5901 | AAAGGCGTTC AAGGACTTCG CCTCGGGAAA ACGTTCTCAG ATAGGGCTCA TTGAGGTTCT GGGAAAGATG CCTGAGCACT |
| | TTTCCGCAAG TTCCTGAAGC GGAGCCCTTT TGCAAGAGTC TATCCCGAGT AACTCCAAGA CCCTTTCTAC GGACTCGTGA |
| | NS4A |
| | --------------------- |
| | M G K T W E |
| | TCATGGGGAA GACATGGGAA |
| | AGTACCCCTT CTGTACCCTT |
| | NS4A |
| | ---------------------------------------------------------------------------------------- |
| | A L D T M Y V V A T A E K G G R A H R M A L E E L P D |
| 6001 | GCACTTGACA CCATGTACGT TGTGGCCACT GCAGAGAAAG GAGGAAGAGC TCACAGAATG GCCCTGGAGG AACTGCCAGA |
| | CGTGAACTGT GGTACATGCA ACACCGGTGA CGTCTCTTTC CTCCTTCTCG AGTGTCTTAC CGGGACCTCC TTGACGGTCT |
| | NS4A |
| | --------------------- |
| | A L Q T I A L • |
| | TGCTCTTCAG ACAATTGCCT |
| | ACGAGAAGTC TGTTAACGGA |
| | NS4A |
| | ---------------------------------------------------------------------------------------- |
| | • I A L L S V M T M G V F F L L M Q R K G I G K I G L |
| 6101 | TGATTGCCTT ATTGAGTGTG ATGACCATGG GAGTATTCTT CCTCCTCATG CAGCGGAAGG GCATTGGAAA GATAGGTTTG |
| | ACTAACGGAA TAACTCACAC TACTGGTACC CTCATAAGAA GGAGGAGTAC GTCGCCTTCC CGTAACCTTT CTATCCAAAC |
| | NS4A |
| | --------------------- |
| | G G A V L G V • |
| | GGAGGCGCTG TCTTGGGAGT |
| | CCTCCGCGAC AGAACCCTCA |
| | NS4A |
| | ---------------------------------------------------------------------------------------- |
| | • A T F F C W M A E V P G T K I A G M L L L S L L L M I |
| 6201 | CGCGACCTTT TTCTGTTGGA TGGCTGAAGT TCCAGGAACG AAGATCGCCG GAATGTTGCT GCTCTCCCTT CTCTTGATGA |
| | GCGCTGGAAA AAGACAACCT ACCGACTTCA AGGTCCTTGC TTCTAGCGGC CTTACAACGA CGAGAGGGAA GAGAACTACT |
| | NS4A |
| | --------------------- |
| | V L I P E P |
| | TTGTGCTAAT TCCTGAGCCA |
| | AACACGATTA AGGACTCGGT |
| | NS4A |
| | ------------------------------------------------------------------------------------------ |
| | E K Q R S Q T D N Q L A V F L I C V M T L V S A V A A |
| 6301 | GAGAAGCAAC GTTCGCAGAC AGACAACCAG CTAGCCGTGT TCCTGATATG TGTCATGACC CTTGTGAGCG CAGTGGCAGC C |
| | CTCTTCGTTG CAAGCGTCTG TCTGTTGGTC GATCGGCACA AGGACTATAC ACAGTACTGG GAACACTCGC GTCACCGTCG G |
| | NS4B |
| | --------------------- |
| | N E M G W L D • |
| | AACGAGATG GGTTGGCTAG |
| | TTGCTCTAC CCAACCGATC |
| | NS4B |
| | ---------------------------------------------------------------------------------------- |
| | • K T K S D I S S L F G Q R I E V K E N F S M G E F L |
| 6401 | ATAAGACCAA GAGTGACATA AGCAGTTTGT TTGGGCAAAG AATTGAGGTC AAGGAGAATT TCAGCATGGG AGAGTTTCTT |
| | TATTCTGGTT CTCACTGTAT TCGTCAAACA AACCCGTTTC TTAACTCCAG TTCCTCTTAA AGTCGTACCC TCTCAAAGAA |
| | NS4B |
| | --------------------- |
| | L D L R P A T • |
| | CTGGACTTGA GGCCGGCAAC |
| | GACCTGAACT CCGGCCGTTG |
| | NS4B |
| | ---------------------------------------------------------------------------------------- |
| | • A W S L Y A V T T A V L T P L L K H L I T S D Y I N T |
| 6501 | AGCCTGGTCA CTGTACGCTG TGACAACAGC GGTCCTCACT CCACTGCTAA AGCATTTGAT CACGTCAGAT TACATCAACA |
| | TCGGACCAGT GACATGCGAC ACTGTTGTCG CCAGGAGTGA GGTGACGATT TCGTAAACTA GTGCAGTCTA ATGTAGTTGT |
| | NS4B |
| | --------------------- |
| | S L T S I N |
| | CCTCATTGAC CTCAATAAAC |
| | GGAGTAACTG GAGTTATTTG |
| | NS4B |
| | ---------------------------------------------------------------------------------------- |
| | V Q A S A L F T L A R G F P F V D V G V S A L L L A A |
| 6601 | GTTCAGGCAA GTGCACTATT CACACTCGCG CGAGGCTTCC CCTTCGTCGA TGTTGGAGTG TCGGCTCTCC TGCTAGCAGC |
| | CAAGTCCGTT CACGTGATAA GTGTGAGCGC GCTCCGAAGG GGAAGCAGCT ACAACCTCAC AGCCGAGAGG ACGATCGTCG |
| | NS4B |
| | --------------------- |
| | G C W G Q V T • |
| | CGGATGCTGG GGACAAGTCA |
| | GCCTACGACC CCTGTTCAGT |
| | NS4B |
| | ---------------------------------------------------------------------------------------- |
| | • L T V T V T A A T L L F C H Y A Y M V P G W Q A E A |
| 6701 | CCCTCACCGT TACGGTAACA GCGGCAACAC TCCTTTTTTG CCACTATGCC TACATGGTTC CCGGTTGGCA AGCTGAGGCA |
| | GGGAGTGGCA ATGCCATTGT CGCCGTTGTG AGGAAAAAAC GGTGATACGG ATGTACCAAG GGCCAACCGT TCGACTCCGT |
| | NS4B |
| | --------------------- |
| | M R S A Q R R • |
| | ATGCGCTCAG CCCAGCGGCG |
| | TACGCGAGTC GGGTCGCCGC |
| | NS4B |
| | ---------------------------------------------------------------------------------------- |
| | • T A A G I M K N A V V D G I V A T D V P E L E R T T P |
| 6801 | GACAGCGGCC GGAATCATGA AGAACGCTGT AGTGGATGGC ATCGTGGCCA CGGACGTCCC AGAATTAGAG CGCACCACAC |
| | CTGTCGCCGG CCTTAGTACT TCTTGCGACA TCACCTACCG TAGCACCGGT GCCTGCAGGG TCTTAATCTC GCGTGGTGTG |
| | NS4B |
| | --------------------- |
| | I M Q K K I |
| | CCATCATGCA GAAGAAAATT |
| | GGTAGTACGT CTTCTTTTAA |
| | NS4B |
| | ---------------------------------------------------------------------------------------- |
| | G Q I M L I L V S L A A V V V N P S V K T V R E A G I |
| 6901 | GGACAGATCA TGCTGATCTT GGTGTCTCTA GCTGCAGTAG TAGTGAACCC GTCTGTGAAG ACAGTACGAG AAGCCGGAAT |
| | CCTGTCTAGT ACGACTAGAA CCACAGAGAT CGACGTCATC ATCACTTGGG CAGACACTTC TGTCATGCTC TTCGGCCTTA |
| | NS4B |
| | --------------------- |
| | L I T A A A V • |
| | TTTGATCACG GCCGCAGCGG |
| | AAACTAGTGC CGGCGTCGCC |
| | NS4B |
| | ---------------------------------------------------------------------------------------- |
| | • T L W E N G A S S V W N A T T A I G L C H I M R G G |
| 7001 | TGACGCTTTG GGAGAATGGA GCAAGCTCTG TTTGGAACGC AACAACTGCC ATCGGACTCT GCCACATCAT GCGTGGGGGT |
| | ACTGCGAAAC CCTCTTACCT CGTTCGAGAC AAACCTTGCG TTGTTGACGG TAGCCTGAGA CGGTGTAGTA CGCACCCCCA |
| | NS4B |
| | --------------------- |
| | W L S C L S I • |
| | TGGTTGTCAT GTCTATCCAT |
| | ACCAACAGTA CAGATAGGTA |
| | NS5 |
| | -------------------------------------- |
| | NS4B |
| | -------------------------------------------------- |
| | • T W T L I K N M E K P G L K R G G A K G R T L G E V W |
| 7101 | AACATGGACA CTCATAAAGA ACATGGAAAA ACCAGGACTA AAAAGAGGTG GGGCAAAAGG ACGCACCTTG GGAGAGGTTT |
| | TTGTACCTGT GAGTATTTCT TGTACCTTTT TGGTCCTGAT TTTTCTCCAC CCCGTTTTCC TGCGTGGAAC CCTCTCCAAA |
| | NS5 |
| | --------------------- |
| | K E R L N Q |
| | GGAAAGAAAG ACTCAACCAG |
| | CCTTTCTTTC TGAGTTGGTC |
| | NS5 |
| | ---------------------------------------------------------------------------------------- |
| | M T K E E F T R Y R K E A I I E V D R S A A K H A R K |
| 7201 | ATGACAAAAG AAGAGTTCAC TAGGTACCGC AAAGAGGCCA TCATCGAAGT CGATCGCTCA GCGGCAAAAC ACGCCAGGAA |
| | TACTGTTTTC TTCTCAAGTG ATCCATGGCG TTTCTCCGGT AGTAGCTTCA GCTAGCGAGT CGCCGTTTTG TGCGGTCCTT |
| | NS5 |
| | --------------------- |
| | E G N V T G G • |
| | AGAAGGCAAT GTCACTGGAG |
| | TCTTCCGTTA CAGTGACCTC |
| | NS5 |
| | ---------------------------------------------------------------------------------------- |
| | • H P V S R G T A K L R W L V E R R F L E P V G K V I |
| 7301 | GGCATCCAGT CTCTAGGGGC ACAGCAAAAC TGAGATGGCT GGTCGAACGG AGGTTTCTCG AACCGGTCGG AAAAGTGATT |
| | CCGTAGGTCA GAGATCCCCG TGTCGTTTTG ACTCTACCGA CCAGCTTGCC TCCAAAGAGC TTGGCCAGCC TTTTCACTAA |
| | NS5 |
| | --------------------- |
| | D L G C G R G • |
| | GACCTTGGAT GTGGAAGAGG |
| | CTGGAACCTA CACCTTCTCC |
| | NS5 |
| | ---------------------------------------------------------------------------------------- |
| | • G W C Y Y M A T Q K R V Q E V R G Y T K G G P G H E E |
| 7401 | CGGTTGGTGT TACTATATGG CAACCCAAAA AAGAGTCCAA GAAGTCAGAG GGTACACAAA GGGCGGTCCC GGACATGAAG |
| | GCCAACCACA ATGATATACC GTTGGGTTTT TTCTCAGGTT CTTCAGTCTC CCATGTGTTT CCCGCCAGGG CCTGTACTTC |
| | NS5 |
| | --------------------- |
| | P Q L V Q S |
| | AGCCCCAACT AGTGCAAAGT |
| | TCGGGGTTGA TCACGTTTCA |
| | NS5 |
| | ---------------------------------------------------------------------------------------- |
| | Y G W N I V T M K S G V D V F Y R P S E C C D T L L C |
| 7501 | TATGGATGGA ACATTGTCAC CATGAAGAGT GGAGTGGATG TGTTCTACAG ACCTTCTGAG TGTTGTGACA CCCTCCTTTG |
| | ATACCTACCT TGTAACAGTG GTACTTCTCA CCTCACCTAC ACAAGATGTC TGGAAGACTC ACAACACTGT GGGAGGAAAC |
| | NS5 |
| | --------------------- |
| | D I G E S S S • |
| | TGACATCGGA GAGTCCTCGT |
| | ACTGTAGCCT CTCAGGAGCA |
| | NS5 |
| | ---------------------------------------------------------------------------------------- |
| | • S A E V E E H R T I R V L E M V E D W L H R G P R E |
| 7601 | CAAGTGCTGA GGTTGAAGAG CATAGGACGA TTCGGGTCCT TGAAATGGTT GAGGACTGGC TGCACCGAGG GCCAAGGGAA |
| | GTTCACGACT CCAACTTCTC GTATCCTGCT AAGCCCAGGA ACTTTACCAA CTCCTGACCG ACGTGGCTCC CGGTTCCCTT |
| | NS5 |
| | --------------------- |
| | F C V K V L C • |
| | TTTTGCGTGA AGGTGCTCTG |
| | AAAACGCACT TCCACGAGAC |
| | NS5 |
| | ---------------------------------------------------------------------------------------- |
| | • P Y M P K V I E K M E L L Q R R Y G G G L V R N P L S |
| 7701 | CCCCTACATG CCGAAAGTCA TAGAGAAGAT GGAGCTGCTC CAACGCCGGT ATGGGGGGGG ACTGGTCAGA AACCCACTCT |
| | GGGGATGTAC GGCTTTCAGT ATCTCTTCTA CCTCGACGAG GTTGCGGCCA TACCCCCCCC TGACCAGTCT TTGGGTGAGA |
| | NS5 |
| | --------------------- |
| | R N S T H E |
| | CACGGAATTC CACGCACGAG |
| | GTGCCTTAAG GTGCGTGCTC |
| | NS5 |
| | ---------------------------------------------------------------------------------------- |
| | M Y W V S R A S G N V V H S V N M T S Q V L L G R M E |
| 7801 | ATGTATTGGG TGAGTCGAGC TTCAGGCAAT GTGGTACATT CAGTGAATAT GACCAGCCAG GTGCTCCTAG GAAGAATGGA |
| | TACATAACCC ACTCAGCTCG AAGTCCGTTA CACCATGTAA GTCACTTATA CTGGTCGGTC CACGAGGATC CTTCTTACCT |
| | NS5 |
| | --------------------- |
| | K R T W K G P • |
| | AAAAAGGACC TGGAAGGGAC |
| | TTTTTCCTGG ACCTTCCCTG |
| | NS5 |
| | ---------------------------------------------------------------------------------------- |
| | • Q Y E E D V N L G S G T R A V G K P L L N S D T S K |
| 7901 | CCCAATACGA GGAAGACGTA AACTTGGGAA GTGGAACCAG GGCGGTGGGA AAACCCCTGC TCAACTCAGA CACCAGTAAA |
| | GGGTTATGCT CCTTCTGCAT TTGAACCCTT CACCTTGGTC CCGCCACCCT TTTGGGGACG AGTTGAGTCT GTGGTCATTT |
| | NS5 |
| | --------------------- |
| | I K N R I E R • |
| | ATCAAGAACA GGATTGAACG |
| | TAGTTCTTGT CCTAACTTGC |
| | NS5 |
| | ---------------------------------------------------------------------------------------- |
| | • L R R E Y S S T W H H D E N H P Y R T W N Y H G S Y D |
| 8001 | ACTCAGGCGT GAGTACAGTT CGACGTGGCA CCACGATGAG AACCACCCAT ATAGAACCTG GAACTATCAT GGCAGTTATG |
| | TGAGTCCGCA CTCATGTCAA GCTGCACCGT GGTGCTACTC TTGGTGGGTA TATCTTGGAC CTTGATAGTA CCGTCAATAC |
| | NS5 |
| | --------------------- |
| | V K P T G S |
| | ATGTGAAGCC CACAGGCTCC |
| | TACACTTCGG GTGTCCGAGG |
| | NS5 |
| | ---------------------------------------------------------------------------------------- |
| | A S S L V N G V V R L L S K P W D T I T N V T T M A M |
| 8101 | GCCAGTTCGC TGGTCAATGG AGTGGTCAGG CTCCTCTCAA AACCATGGGA CACCATCACG AATGTTACCA CCATGGCCAT |
| | CGGTCAAGCG ACCAGTTACC TCACCAGTCC GAGGAGAGTT TTGGTACCCT GTGGTAGTGC TTACAATGGT GGTACCGGTA |
| | NS5 |
| | --------------------- |
| | T D T T P F G • |
| | GACTGACACT ACTCCCTTCG |
| | CTGACTGTGA TGAGGGAAGC |
| | NS5 |
| | ---------------------------------------------------------------------------------------- |
| | • Q Q R V F K E K V D T K A P E P P E G V K Y V L N E |
| 8201 | GGCAGCAGCG AGTGTTCAAA GAGAAGGTGG ACACGAAAGC TCCTGAACCG CCAGAAGGAG TGAAGTACGT GCTCAACGAG |
| | CCGTCGTCGC TCACAAGTTT CTCTTCCACC TGTGCTTTCG AGGACTTGGC GGTCTTCCTC ACTTCATGCA CGAGTTGCTC |
| | NS5 |
| | --------------------- |
| | T T N W L W A • |
| | ACCACCAACT GGTTGTGGGC |
| | TGGTGGTTGA CCAACACCCG |
| | NS5 |
| | ---------------------------------------------------------------------------------------- |
| | • F L A R E K R P R M C S R E E F I R K V N S N A A L G |
| 8301 | GTTTTTGGCC AGAGAAAAAC GTCCCAGAAT GTGCTCTCGA GAGGAATTCA TAAGAAAGGT CAACAGCAAT GCAGCTTTGG |
| | CAAAAACCGG TCTCTTTTTG CAGGGTCTTA CACGAGAGCT CTCCTTAAGT ATTCTTTCCA GTTGTCGTTA CGTCGAAACC |
| | NS5 |
| | --------------------- |
| | A M F E E Q |
| | GTGCCATGTT TGAAGAGCAG |
| | CACGGTACAA ACTTCTCGTC |
| | NS5 |
| | ---------------------------------------------------------------------------------------- |
| | N Q W R S A R E A V E D P K F W E M V D E E R E A H L |
| 8401 | AATCAATGGA GGAGCGCCAG AGAAGCAGTT GAAGATCCAA AATTTTGGGA AATGGTGGAT GAGGAGCGCG AGGCACATCT |
| | TTAGTTACCT CCTCGCGGTC TCTTCGTCAA CTTCTAGGTT TTAAAACCCT TTACCACCTA CTCCTCGCGC TCCGTGTAGA |
| | NS5 |
| | --------------------- |
| | R G E C H T C • |
| | GCGGGGGGAA TGTCACACTT |
| | CGCCCCCCTT ACAGTGTGAA |
| | NS5 |
| | ---------------------------------------------------------------------------------------- |
| | • I Y N M M G K R E K K P G E F G K A K G S R A I W F |
| 8501 | GCATTTACAA CATGATGGGA AAGAGAGAGA AAAAACCCGG AGAGTTCGGA AAGGCCAAGG GAAGCAGAGC CATTTGGTTC |
| | CGTAAATGTT GTACTACCCT TTCTCTCTCT TTTTTGGGCC TCTCAAGCCT TTCCGGTTCC CTTCGTCTCG GTAAACCAAG |
| | NS5 |
| | --------------------- |
| | M W L G A R F • |
| | ATGTGGCTCG GAGCTCGCTT |
| | TACACCGAGC CTCGAGCGAA |
| | NS5 |
| | ---------------------------------------------------------------------------------------- |
| | • L E F E A L G F L N E D H W L G R K N S G G G V E G L |
| 8601 | TCTGGAGTTC GAGGCTCTGG GTTTTCTCAA TGAAGACCAC TGGCTTGGAA GAAAGAACTC AGGAGGAGGT GTCGAGGGCT |
| | AGACCTCAAG CTCCGAGACC CAAAAGAGTT ACTTCTGGTG ACCGAACCTT CTTTCTTGAG TCCTCCTCCA CAGCTCCCGA |
| | NS5 |
| | --------------------- |
| | G L Q K L G |
| | TGGGCCTCCA AAAACTGGGT |
| | ACCCGGAGGT TTTTGACCCA |
| | NS5 |
| | ---------------------------------------------------------------------------------------- |
| | Y I L R E V G I R P G G K I Y A D D T A G W D T R I T |
| 8701 | TACATCCTGC GTGAAGTTGG CATCCGGCCT GGGGGCAAGA TCTATGCTGA TGACACAGCT GGCTGGGACA CCCGCATCAC |
| | ATGTAGGACG CACTTCAACC GTAGGCCGGA CCCCCGTTCT AGATACGACT ACTGTGTCGA CCGACCCTGT GGGCGTAGTG |
| | NS5 |
| | --------------------- |
| | R A D L E N E • |
| | GAGAGCTGAC TTGGAAAATG |
| | CTCTCGACTG AACCTTTTAC |
| | NS5 |
| | ---------------------------------------------------------------------------------------- |
| | • A K V L E L L D G E H R R L A R A I I E L T Y R H K |
| 8801 | AAGCTAAGGT GCTTGAGCTG CTTGATGGGG AACATCGGCG TCTTGCCAGG GCCATCATTG AGCTCACCTA TCGTCACAAA |
| | TTCGATTCCA CGAACTCGAC GAACTACCCC TTGTAGCCGC AGAACGGTCC CGGTAGTAAC TCGAGTGGAT AGCAGTGTTT |
| | NS5 |
| | --------------------- |
| | V V K V M R P • |
| | GTTGTGAAAG TGATGCGCCC |
| | CAACACTTTC ACTACGCGGG |
| | NS5 |
| | ---------------------------------------------------------------------------------------- |
| | • A A D G R T V M D V I S R E D Q R G S G Q V V T Y A L |
| 8901 | GGCTGCTGAT GGAAGAACCG TTATGGATGT TATCTCCAGA GAAGATCAGA GGGGGAGTGG ACAAGTTGTC ACCTACGCCC |
| | CCGACGACTA CCTTCTTGGC AATACCTACA ATAGAGGTCT CTTCTAGTCT CCCCCTCACC TGTTCAACAG TGGATGCGGG |
| | NS5 |
| | --------------------- |
| | N T F T N L |
| | TAAACACTTT CACCAACCTG |
| | ATTTGTGAAA GTGGTTGGAC |
| | NS5 |
| | ---------------------------------------------------------------------------------------- |
| | A V Q L V R M M E G E G V I G P D D V E K L T K G K G |
| 9001 | GCTGTCCAGC TGGTGAGGAT GAATGAATGG GAAGGAGTGA TTGGCCCAGA TGATGTGGAG AAACTCACAA AAGGGAAAGG |
| | CGACAGGTCG ACCACTCCTA CTACCTTCCC CTTCCTCACT AACCGGGTCT ACTACACCTC TTTGAGTGTT TTCCCTTTCC |
| | NS5 |
| | --------------------- |
| | P K V R T W L • |
| | ACCCAAAGTC AGGACCTGGC |
| | TGGGTTTCAG TCCTGGACCG |
| | NS5 |
| | ---------------------------------------------------------------------------------------- |
| | • F E N G E E R L S R M A V S G D D C V V K P L D D R |
| 9101 | TGTTTGAGAA TGGGGAAGAA AGACTCAGCC GCATGGCTGT CAGTGGAGAT GACTGTGTGG TAAAGCCCCT GGACGATCGC |
| | ACAAACTCTT ACCCCTTCTT TCTGAGTCGG CGTACCGACA GTCACCTCTA CTGACACACC ATTTCGGGGA CCTGCTAGCG |
| | NS5 |
| | --------------------- |
| | F A T S L H F • |
| | TTTGCCACCT CGCTCCACTT |
| | AAACGGTGGA GCGAGGTGAA |
| | NS5 |
| | ---------------------------------------------------------------------------------------- |
| | • L N A M S K V R K D I Q E W K P S T G W Y D W Q Q V P |
| 9201 | CCTCAATGCT ATGTCAAAGG TTCGCAAAGA CATCCAAGAG TGGAAACCGT CAACTGGATG GTATGATTGG CAGCAGGTTC |
| | GGAGTTACGA TACAGTTTCC AAGCGTTTCT GTAGGTTCTC ACCTTTGGCA GTTGACCTAC CATACTAACC GTCGTCCAAG |
| | NS5 |
| | --------------------- |
| | F C S N H F |
| | CATTTTGCTC AAACCATTTC |
| | GTAAAACGAG TTTGGTAAAG |
| | NS5 |
| | ---------------------------------------------------------------------------------------- |
| | T E L I M K D G R T L V V P C R G Q D E L V G R A R I |
| 9301 | ACTGAATTGA TCATGAAAGA TGGAAGAACA CTGGTGGTTC CATGCCGAGG ACAGGATGAA TTGGTAGGCA GAGCTCGCAT |
| | TGACTTAACT AGTACTTTCT ACCTTCTTGT GACCACCAAG GTACGGCTCC TGTCCTACTT AACCATCCGT CTCGAGCGTA |
| | NS5 |
| | --------------------- |
| | S P G A G W N • |
| | ATCTCCAGGG GCCGGATGGA |
| | TAGAGGTCCC CGGCCTACCT |
| | NS5 |
| | ---------------------------------------------------------------------------------------- |
| | • V R D T A C L A K S Y A Q M W L L L Y F H R R D L R |
| 9401 | ACGTCCGCGA CACTGCTTGT CTGGCTAAGT CTTATGCCCA GATGTGGCTG CTTCTGTACT TCCACAGAAG AGACCTGCGG |
| | TGCAGGCGCT GTGACGAACA GACCGATTCA GAATACGGGT CTACACCGAC GAAGACATGA AGGTGTCTTC TCTGGACGCC |
| | NS5 |
| | --------------------- |
| | L M A N A I C • |
| | CTCATGGCCA ACGCCATTTG |
| | GAGTACCGGT TGCGGTAAAC |
| | NS5 |
| | ---------------------------------------------------------------------------------------- |
| | • S A V P V N W V P T G R T T W S I H A G G E W M T T E |
| 9501 | CTCCGCTGTC CCTGTGAATT GGGTCCCTAC CGGAAGAACC ACGTGGTCCA TCCATGCAGG AGGAGAGTGG ATGACAACAG |
| | GAGGCGACAG GGACACTTAA CCCAGGGATG GCCTTCTTGG TGCACCAGGT AGGTACGTCC TCCTCTCACC TACTGTTGTC |
| | NS5 |
| | --------------------- |
| | D M L E V W |
| | AGGACATGTT GGAGGTCTGG |
| | TCCTGTACAA CCTCCAGACC |
| | NS5 |
| | ---------------------------------------------------------------------------------------- |
| | N R V W I E E N E W M E D K T P V E K W S D V P Y S G |
| 9601 | AACCGTGTTT GGATAGAGGA GAATGAATGG ATGGAAGACA AAACCCCAGT GGAGAAATGG AGTGACGTCC CATATTCAGG |
| | TTGGCACAAA CCTATCTCCT CTTACTTACC TACCTTCTGT TTTGGGGTCA CCTCTTTACC TCACTGCAGG GTATAAGTCC |
| | NS5 |
| | --------------------- |
| | K R E D I W C • |
| | AAAACGAGAG GACATCTGGT |
| | TTTTGCTCTC CTGTAGACCA |
| | NS5 |
| | ---------------------------------------------------------------------------------------- |
| | • G S L I G T R A R A T W A E N I Q V A I N Q V R A I |
| 9701 | GTGGCAGCCT GATTGGCACA AGAGCCCGAG CCACGTGGGC AGAAAACATC CAGGTGGCTA TCAACCAAGT CAGAGCAATC |
| | CACCGTCGGA CTAACCGTGT TCTCGGGCTC GGTGCACCCG TCTTTTGTAG GTCCACCGAT AGTTGGTTCA GTCTCGTTAG |
| | NS5 |
| | --------------------- |
| | I G D E K Y V • |
| | ATCGGAGATG AGAAGTATGT |
| | TAGCCTCTAC TCTTCATACA |
| | 3′ UTR |
| | --------------------- |
| | NS5 |
| | ------------------------------------------------------------------- |
| | • D Y M S S L K R Y E D T T L V E D T V L |
| 9801 | GGATTACATG AGTTCACTAA AGAGATATGA AGACACAACT TTGGTTGAGG ACACAGTACT GTAGATATTT AATCAATTGT |
| | CCTAATGTAC TCAAGTGATT TCTCTATACT TCTGTGTTGA AACCAACTCC TGTGTCATGA CATCTATAAA TTAGTTAACA |
| | 3′ UTR |
| | --------------------- |
| | AAATAGACAA TATAAGTATG |
| | TTTATCTGTT ATATTCATAC |
| | 3′ UTR |
| | ---------------------------------------------------------------------------------------- |
| 9901 | CATAAAAGTG TAGTTTTATA GTAGTATTTA GTGGTGTTAG TGTAAATAGT TAAGAAAATC TTGAGGAGAA AGTCAGGCCG |
| | GTATTTTCAC ATCAAAATAT CATCATAAAT CACCACAATC ACATTTATCA ATTCTTTTAG AACTCCTCTT TCAGTCCGGC |
| | 3′ UTR |
| | --------------------- |
| | GGAAGTTCCC GCCACCGGAA |
| | CCTTCAAGGG CGGTGGCCTT |
| | 3′ UTR |
| | ---------------------------------------------------------------------------------------- |
| 10001 | GTTGAGTAGA CGGTGCTGCC TGCGACTCAA CCCCAGGAGG ACTGGGTGAA CAAAGCCGCG AAGTGATCCA TGTAAGCCCT |
| | CAACTCATCT GCCACGACGG ACGCTGAGTT GGGGTCCTCC TGACCCACTT GTTTCGGCGC TTCACTAGGT ACATTCGGGA |
| | 3′ UTR |
| | --------------------- |
| | CAGAACCGTC TCGGAAGGAG |
| | GTCTTGGCAG AGCCTTCCTC |
| | 3′ UTR |
| | ---------------------------------------------------------------------------------------- |
| 10101 | GACCCCACAT GTTGTAACTT CAAAGCCCAA TGTCAGACCA CGCTACGGCG TGCTACTCTG CGGAGAGTGC AGTCTGCGAT |
| | CTGGGGTGTA CAACATTGAA GTTTCGGGTT ACAGTCTGGT GCGATGCCGC ACGATGAGAC GCCTCTCACG TCAGACGCTA |
| | 3′ UTR |
| | --------------------- |
| | AGTGCCCCAG GAGGACTGGG |
| | TCACGGGGTC CTCCTGACCC |
| | 3′ UTR |
| | ---------------------------------------------------------------------------------------- |
| 10201 | TTAACAAAGG CAAACCAACG CCCCACGCGG CCCAAGCCCC GGTAATGGTG TTAACCAGGG CGAAAGGACT AGAGGTTAGA |
| | AATTGTTTCC GTTTGGTTGC GGGGTGCGCC GGGTTCGGGG CCATTACCAC AATTGGTCCC GCTTTCCTGA TCTCCAATCT |
| | 3′ UTR |
| | --------------------- |
| | GGAGACCCCG CGGTTTAAAG |
| | CCTCTGGGGC GCCAAATTTC |
| | 3′ UTR |
| | ---------------------------------------------------------------------------------------- |
| 10301 | TGCACGGCCC AGCCTGGCTG AAGCTGTAGG TCAGGGGAAG GACTAGAGGT TAGTGGAGAC CCCGTGCCAC AAAACACCAC |
| | ACGTGCCGGG TCGGACCGAC TTCGACATCC AGTCCCCTTC CTGATCTCCA ATCACCTCTG GGGCACGGTG TTTTGTGGTG |
| | 3′ UTR |
| | --------------------- |
| | AACAAAACAG CAAATAGACA |
| | TTGTTTTGTC GTTTATCTGT |
| | 3′ UTR |
| | ---------------------------------------------------------------------------------------- |
| 10401 | CCTGGGATAG ACTAGGAGAT CTTCTGCTCT GCACAACCAG CCACACGGCA CAGTGCGCCG ACAATGGTGG CTGGTGGTGC |
| | GGACCCTATC TGATCCTCTA GAAGACGAGA CGTGTTGGTC GGTGTGCCGT GTCACGCGGC TGTTACCACC GACCACCACG |
| | 3′ UTR |
| | ---------------- |
| | GAGAACACAG GATCT |
| | CTCTTGTGTC CTAGA |

SEQUENCE APPENDIX 7
Sequence of RSV G

```
                  10 19 28 37 46 55
TGCAAAC ATG TCC AAA AAC AAG GAC CAA CGC ACC GCT AAG ACA CTA GAA AAG ACC
                MET Ser Lys Asn Lys Asp Gln Arg Thr Ala Lys Thr Leu Glu Lys Thr
                  64 73 82 91 100 109
TGG GAC ACT CTC AAT CAT TTA TTA TTC ATA TCA TCG GGC TTA TAT AAG TTA AAT
Trp Asp Thr Leu Asn His Leu Leu Phe Ile Ser Ser Gly Leu Tyr Lys Leu Asn
                118 127 136 145 154 163
CTT AAA TCT GTA GCA CAA ATC ACA TTA TCC ATT CTG GCA ATG ATA ATC TCA ACT
Leu Lys Ser Val Ala Gln Ile Thr Leu Ser Ile Leu Ala MET Ile Ile Ser Thr
                172 181 190 199 208 217
TCA CTT ATA ATT ACA GCC ATC ATA TTC ATA GCC TCG GCA AAC CAC AAA GTC ACA
Ser Leu Ile Ile Thr Ala Ile Ile Phe Ile Ala Ser Ala Asn His Lys Val Thr
                226 235 244 253 262 271
CTA ACA ACT GCA ATC ATA CAA GAT GCA ACA AGC CAG ATC AAG AAC ACA ACC CCA
Leu Thr Thr Ala Ile Ile Gln Asp Ala Thr Ser Gln Ile Lys Asn Thr Thr Pro
                280 289 298 307 316 325
ACA TAC CTC ACT CAG GAT CCT CAG CTT GGA ATC AGC TTC TCC AAT CTG TCT GAA
Thr Tyr Leu Thr Gln Asp Pro Gln Leu Gly Ile Ser Phe Ser Asn Leu Ser Glu
                334 343 352 361 370 379
ATT ACA TCA CAA ACC ACC ACC ATA CTA GCT TCA ACA ACA CCA GGA GTC AAG TCA
Ile Thr Ser Gln Thr Thr Thr Ile Leu Ala Ser Thr Thr Pro Gly Val Lys Ser
                388 397 406 415 424 433
AAC CTG CAA CCC ACA ACA GTC AAG ACT AAA AAC ACA ACA ACA ACC CAA ACA CAA
Asn Leu Gln Pro Thr Thr Val Lys Thr Lys Asn Thr Thr Thr Thr Gln Thr Gln
                442 451 460 469 478 487
CCC AGC AAG CCC ACT ACA AAA CAA CGC CAA AAC AAA CCA CCA AAC AAA CCC AAT
Pro Ser Lys Pro Thr Thr Lys Gln Arg Gln Asn Lys Pro Pro Asn Lys Pro Asn
                496 505 514 523 532 541
AAT GAT TTT CAC TTC GAA GTG TTT AAC TTT GTA CCC TGC AGC ATA TGC AGC AAC
Asn Asp Phe His Phe Glu Val Phe Asn Phe Val Pro Cys Ser Ile Cys Ser Asn
                550 559 568 577 586 595
AAT CCA ACC TGC TGG GCT ATC TGC AAA AGA ATA CCA AAC AAA AAA CCA GGA AAG
Asn Pro Thr Cys Trp Ala Ile Cys Lys Arg Ile Pro Asn Lys Lys Pro Gly Lys
                604 613 622 631 640 649
AAA ACC ACC ACC AAG CCT ACA AAA AAA CCA ACC TTC AAG ACA ACC AAA AAA GAT
Lys Thr Thr Thr Lys Pro Thr Lys Lys Pro Thr Phe Lys Thr Thr Lys Lys Asp
                658 667 676 685 694 703
CTC AAA CCT CAA ACC ACT AAA CCA AAG GAA GTA CCC ACC ACC AAG CCC ACA GAA
Leu Lys Pro Gln Thr Thr Lys Pro Lys Glu Val Pro Thr Thr Lys Pro Thr Glu
                712 721 730 739 748 757
GAG CCA ACC ATC AAC ACC ACC AAA ACA AAC ATC ACA ACT ACA CTG CTC ACC AAC
Glu Pro Thr Ile Asn Thr Thr Lys Thr Asn Ile Thr Thr Thr Leu Leu Thr Asn
                766 775 784 793 802 811
AAC ACC ACA GGA AAT CCA AAA CTC ACA AGT CAA ATG GAA ACC TTC CAC TCA ACC
Asn Thr Thr Gly Asn Pro Lys Leu Thr Ser Gln MET Glu Thr Phe His Ser Thr
                820 829 838 847 856 865
TCC TCC GAA GGC AAT CTA AGC CCT TCT CAA GTC TCC ACA ACA TCC GAG CAC CCA
Ser Ser Glu Gly Asn Leu Ser Pro Ser Gln Val Ser Thr Thr Ser Glu His Pro
                874 883 892 901 914
TCA CAA CCC TCA TCT CCA CCC AAC ACA ACA CGC CAG TAG TTATTAAAAA AAAAAA
Ser Gln Pro Ser Ser Pro Pro Asn Thr Thr Arg Gln
```

1. A replication-deficient pseudoinfectious flavivirus comprising a flavivirus genome comprising (i) one or more deletions or mutations in nucleotide sequences encoding one or more proteins selected from the group consisting of capsid (C), pre-membrane (prM), envelope (E), non-structural protein 1 (NS1), non-structural protein 3 (NS3), and non-structural protein 5 (NS5), and (ii) a sequence encoding a respiratory syncytial virus (RSV) peptide or protein, or a fragment or analog thereof.

2. The replication-deficient pseudoinfectious flavivirus of claim 1, wherein said respiratory syncytial virus (RSV) protein is the RSV F protein, or a fragment or analog thereof.

3. The replication-deficient pseudoinfectious flavivirus of claim 2, wherein said RSV F protein lacks a trans-membrane domain.

4. The replication-deficient pseudoinfectious flavivirus of claim 3, wherein said RSV F protein is truncated so that it is produced in secreted form.

5. The replication-deficient pseudoinfectious flavivirus of claim 1, wherein said respiratory syncytial virus (RSV) protein is the RSV G protein, or a fragment or analog thereof.

6. The replication-deficient pseudoinfectious flavivirus of claim 1, wherein said one or more deletions or mutations is within capsid (C) sequences of the flavivirus genome.

7. The replication-deficient pseudoinfectious flavivirus of claim 1, wherein said one or more deletions or mutations is within pre-membrane (prM) and/or envelope (E) sequences of the flavivirus genome.

8. The replication-deficient pseudoinfectious flavivirus of claim 1, wherein said one or more deletions or mutations is within capsid (C), pre-membrane (prM), and envelope (E) sequences of the flavivirus genome.

9. The replication-deficient pseudoinfectious flavivirus of claim 1, wherein said one or more deletions or mutations is within non-structural protein 1 (NS1) sequences of the flavivirus genome.

10. The replication-deficient pseudoinfectious flavivirus of claim 1, wherein said flavivirus genome comprises sequences encoding a pre-membrane (prM) and/or envelope (E) protein.

11. The replication-deficient pseudoinfectious flavivirus of claim 1, wherein the flavivirus genome is selected from that of yellow fever virus, West Nile virus, tick-borne encephalitis virus, Langat virus, Japanese encephalitis virus, dengue virus, and St. Louis encephalitis virus sequences, and chimeras thereof.

12. The replication-deficient pseudoinfectious flavivirus of claim 11, wherein said chimera comprises pre-membrane (prM) and envelope (E) sequences of a first flavivirus, and capsid (C) and non-structural sequences of a second, different flavivirus.

13. The replication-deficient pseudoinfectious flavivirus of claim 1, wherein said genome is packaged in a particle comprising pre-membrane (prM) and envelope (E) sequences from a flavivirus that is the same or different from that of the genome.

14. The replication-deficient pseudoinfectious flavivirus of claim 1, wherein sequences encoding said respiratory syncytial virus peptide or protein, or a fragment or analog thereof are inserted in the place of or in combination with the one or more deletions or mutations of the one or more proteins.

15. The replication-deficient pseudoinfectious flavivirus of claim 1, wherein sequences encoding said respiratory syncytial virus peptide or protein, or a fragment or analog thereof are inserted in the flavivirus genome within sequences encoding the envelope (E) protein, within sequences encoding the non-structural 1 (NS1) protein, within sequences encoding the pre-membrane (prM) protein, intergenically between sequences encoding the envelope (E) protein and non-structural protein 1 (NS1), intergenically between non-structural protein 2B (NS2B) and non-structural protein 3 (NS3), or as a bicistronic insertion in the 3′ untranslated region of the flavivirus genome.

16. A composition comprising a first replication-deficient pseudoinfectious flavivirus of claim 1 and a second, different replication-deficient pseudoinfectious flavivirus comprising a genome comprising one or more deletions or mutations in nucleotide sequences encoding one or more proteins selected from the group consisting of capsid (C), pre-membrane (prM), envelope (E), non-structural protein 1 (NS1), non-structural protein 3 (NS3), and non-structural protein 5 (NS5), wherein the one or more proteins encoded by the sequences in which the one or more deletion(s) or mutation(s) occur in the second, different replication-deficient pseudoinfectious flavivirus are different from the one or more proteins encoded by the sequences in which the one or more deletion(s) or mutation(s) occur in the first replication-deficient pseudoinfectious flavivirus.

17. A method of inducing an immune response to respiratory syncytial virus (RSV) in a subject, the method comprising administering to the subject one or more replication-deficient pseudoinfectious flaviviruses of claim 1 to the subject.

18. The method of claim 17, wherein the subject is at risk of but does not have an infection by respiratory syncytial virus (RSV).

19. The method of claim 17, wherein the subject has an infection by respiratory syncytial virus (RSV).

20. The method of claim 17, wherein the subject is an infant, young child, or elderly person.

21. The method of claim 17, wherein the method is for inducing an immune response against a protein encoded by the flavivirus genome, in addition to respiratory syncytial virus.

22. The method of claim 21, wherein the subject is at risk of but does not have an infection by the flavivirus corresponding to the genome of the pseudoinfectious flavivirus, which comprises sequences encoding a flavivirus pre-membrane and/or envelope protein.

23. The method of claim 21, wherein the subject has an infection by the flavivirus corresponding to the genome of the pseudoinfectious flavivirus which comprises sequences encoding a flavivirus pre-membrane and/or envelope protein.

24. A pharmaceutical composition comprising a pseudoinfectious flavivirus of claim 1, and a pharmaceutically acceptable carrier or diluent.

25. The pharmaceutical composition of claim 24, further comprising an adjuvant.

26. A nucleic acid molecule corresponding to the genome of a pseudoinfectious flavivirus of claim 1 or the complement thereof.

27. A method of making a replication-deficient pseudoinfectious flavivirus of claim 1, the method comprising introducing a nucleic acid molecule of claim 26 into a cell that expresses the protein corresponding to any sequences deleted from the flavivirus genome of the replication-deficient pseudoinfectious flavivirus.

28. The method of claim 27, wherein the protein is expressed in the cell from the genome of a second, different, replication-deficient pseudoinfectious flavivirus.

29. The method of claim 27, wherein the protein is expressed from a replicon.

30. The method of claim 29, wherein the replicon is an alphavirus replicon.

31. The method of claim 30, wherein the alphavirus is a Venezuelan Equine Encephalitis virus.


SEQUENCE LISTING (92 sequences)

SEQ ID NO: 1 - protein, 12 aa, Yellow Fever Virus
SHDVLTVQFLIL

SEQ ID NO: 2 - protein, 8 aa, Tick-borne Encephalitis Virus
GMLGMTIA

SEQ ID NO: 3 - protein, 20 aa, Artificial Sequence
  Synthetic Construct
SHDVLTVQFLILGMLGMTIA

SEQ ID NO: 4 - protein, 20 aa, Tick-borne Encephalitis Virus
GGTDWMSWLLVIGMLGMTIA

SEQ ID NO: 5 - protein, 9 aa, Borrelia burgdorferi
YVLEGTLTA

SEQ ID NO: 6 - protein, 9 aa, Borrelia afzelli
FTLEGKVAN

SEQ ID NO: 7 - protein, 9 aa, Artificial Sequence
  Synthetic Construct
FTLEGKLTA

SEQ ID NO: 8 - protein, 273 aa, Borrelia burgdorferi
MKKYLLGIGLILALIACKQNVSSLDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYVLEGTLTAEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALK

SEQ ID NO: 9 - protein, 8 aa, Artificial Sequence
  Synthetic Construct
LPGXXXVL

SEQ ID NO: 10 - protein, 11 aa, Artificial Sequence
  Synthetic Construct
GTSDKXNGSGX

SEQ ID NO: 11 - protein, 31 aa, Artificial Sequence
  Synthetic Construct
XIXXSGEXXXXLXDXXXXXATKKTXXWXXXT

SEQ ID NO: 12 - protein, 21 aa, Artificial Sequence
  Synthetic Construct
SXGTXLEGXAVEIXXLXEXKN

SEQ ID NO: 13 - protein, 154 aa, Rhipicephalus appendiculatus
MKAFFVLSLLSTAALTNAARAGRLGSDLDTFGRVHGNLYAGIERAGPRGYPGLTASIGGEVGARLGGRAGVGVSSYGYGYPSWGYPYGGYGGYGGYGGYGGYDQGFGSAYGGYPGYYGYYYPSGYGGGYGGSYGGSYGGSYTYPNVRASAGAAA

SEQ ID NO: 14 - protein, 184 aa, Ixodes scapularis
MRTAFTCALLAISFLGSPCSSSEDGLEQDTIVETTTQNLYERHYRNHSGLCGAQYRNSSHAEAVYNCTLNHLPPVVNATWEGIRHRINKTIPQFVKLICNFTVAMPQEFYLVYMGSDGNSDFEEDKESTGTDEDSNTGSSAAAKVTEALIIEAEENCTAHITGWTTETPTTLEPTTESQFEAIP

SEQ ID NO: 15 - protein, 177 aa, Ixodes scapularis
MRTALTCALLAISFLGSPCSSSEGGLEKDSRVETTTQNLYERYYRKHPGLCGAQYRNSSHAEAVYNCTLSLLPLSVNTTWEGIRHRINKTIPEFVNLICNFTVAMPDQFYLVYMGSNGNSYSEEDEDGKTGSSAAVQVTEQLIIQAEENCTAHITGWTTEAPTTLEPTTETQFEAIS

SEQ ID NO: 16 - protein, 19 aa, Influenza A virus
PAKLLKERGFFGAIAGFLE

SEQ ID NO: 17 - protein, 23 aa, Influenza A virus
PAKLLKERGFFGAIAGFLEGSGC

SEQ ID NO: 18 - protein, 19 aa, Influenza B virus
NNATFNYTNVNPISHIRGS

SEQ ID NO: 19 - protein, 24 aa, Influenza A virus
MSLLTEVETPIRNEWGCRCNDSSD

SEQ ID NO: 20 - protein, 24 aa, Influenza A virus
MSLLTEVETPTRNEWECRCSDSSD

SEQ ID NO: 21 - protein, 24 aa, Influenza A virus
MSLLTEVETLTRNGWGCRCSDSSD

SEQ ID NO: 22 - protein, 8 aa, Influenza A virus
EVETPTRN

SEQ ID NO: 23 - protein, 23 aa, Influenza A virus
SLLTEVETPIRNEWGCRCNDSSD

SEQ ID NO: 24 - protein, 17 aa, Influenza A virus
SLLTEVETPIRNEWGCR

SEQ ID NO: 25 - protein, 6 aa, Influenza B virus
MLEPFQ

SEQ ID NO: 26 - protein, 12 aa, Influenza B virus
LEPFQILSISGC

SEQ ID NO: 27 - protein, 24 aa, Avian Influenza A virus Subtype H5N1
MSLLTEVETLTRNGWGCRCSDSSD

SEQ ID NO: 28 - protein, 28 aa, Artificial Sequence
  Synthetic Construct
RKRRSHDVLTVQFLILGMLGMTIAATVR

SEQ ID NO: 29 - DNA, 84 bp, Artificial Sequence
  Synthetic Construct
aggaaacgccgttcccatgatgttctgactgtgcaattcctaattttgggcatgctgggcatgacaatcgcagctacggttcgc

SEQ ID NO: 30 - DNA, 84 bp, Artificial Sequence
  Synthetic Construct
tcctttgcggcaagggtactacaagactgacacgttaaggattaaaacccgtacgacccgtactgttagcgtcgatgccaagcg

SEQ ID NO: 31 - protein, 28 aa, Artificial Sequence
  Synthetic Construct
RKRRSHDVLTVQFLILGMLACVGAATVR

SEQ ID NO: 32 - DNA, 84 bp, Artificial Sequence
  Synthetic Construct
aggaaacgccgttcccatgatgttctgactgtgcaattcctaattttgggcatgctggcttgtgtcggagcagctaccgtgcga

SEQ ID NO: 33 - DNA, 84 bp, Artificial Sequence
  Synthetic Construct
tcctttgcggcaagggtactacaagactgacacgttaaggattaaaacccgtacgaccgaacacagcctcgtcgatggcacgct

SEQ ID NO: 34 - protein, 28 aa, Artificial Sequence
  Synthetic Construct
QKKRGGTDWMSWLLVIGMLGMTIAATVR

SEQ ID NO: 35 - DNA, 84 bp, Artificial Sequence
  Synthetic Construct
caaaagaaacgggggggaacagactggatgagctggctgctcgtaatcggcatgctgggcatgacaatcgcagctacggttcgc

SEQ ID NO: 36 - DNA, 84 bp, Artificial Sequence
  Synthetic Construct
gttttctttgcccccccttgtctgacctactcgaccgacgagcattagccgtacgacccgtactgttagcgtcgatgccaagcg

SEQ ID NO: 37 - protein, 26 aa, Artificial Sequence
  Synthetic Construct
QKKRGGKTGIAVMIGMLACVGAATVR

SEQ ID NO: 38 - DNA, 78 bp, Artificial Sequence
  Synthetic Construct
caaaagaaacgcgggggaaagacaggcatagctgtgatgataggcatgctggcttgtgtcggagcagctaccgtgcga

SEQ ID NO: 39 - DNA, 78 bp, Artificial Sequence
  Synthetic Construct
gttttctttgcgccccctttctgtccgtatcgacactactatccgtacgaccgaacacagcctcgtcgatggcacgct

SEQ ID NO: 40 - protein, 793 aa, Artificial Sequence
  Synthetic Construct
MSGRKAQGKTLGVNMVRRGVRSLSNKIKQKTKQIGNRPGPSRGVQGFIFFFLFNILTGKKITAHLKRLWKMLDPRQGLAVLRKVKRVVSLMRGLSSRKRRSHDVLTVQFLILGMLGMTIAATVRKERDGSTVIRAEGKDAATQVRVENGTCVILATDMGSWCDDSLSYECVTIDQGEEPVDVDCFCRNVDGVYLEYGRCGKQEGSRTRRSVLIPSHAQGELTGRGHKWLEGDSLRTHLTRVEGWVWKNRLLALAMVTVVWLTLESVVTRVAVLVVLLCLAPVYASRCTHLENRDFVTGTQGTTRVTLVLELGGCVTITAEGKPSMDVWLDAIYQENPAQTREYCLHAKLSDTKVAARCPTMGPATLAEEHQGGTVCKRDQSDRGWGNHCGLFGKGSIVACVKAACEAKKKATGHVYDANKIVYTVKVEPHTGDYVAANETHSGRKTASFTVSSEKTILTMGEYGDVSLLCRVASGVDLAQTVILELDKTVEHLPTAWQVHRDWFNDLALPWKHEGARNWNNAERLVEFGAPHAVKMDVYNLGDQTGVLLKALAGVPVAHIEGTKYHLKSGHVTCEVGLEKLKMKGLTYTMCDKTKFTWKRAPTDSGHDTVVMEVTFSGTKPCRIPVRAVAHGSPDVNVAMLITPNPTIENNGGGFIEMQLPPGDNIIYVGELSYQWFQKGSSIGRVFQKTKKGIERLTVIGEHAWDFGSAGGFLSSIGKALHTVLGGAFNSIFGGVGFLPKLLLGVALAWLGLNMRNPTMSMSFLLAGVLVLAMTLGVGADQGCAINFGKREL

SEQ ID NO: 41 - DNA, 2500 bp, Artificial Sequence
  Synthetic Construct
agtaaatcctgtgtgctaattgaggtgcattggtctgcaaatcgagttgctaggcaataaacacatttggattaattttaatcgttcgttgagcgattagcagagaactgaccagaacatgtctggtcgtaaagctcagggaaaaaccctgggcgtcaatatggtacgacgaggagttcgctccttgtcaaacaaaataaaacaaaaaacaaaacaaattggaaacagacctggaccttcaagaggtgttcaaggatttatctttttctttttgttcaacattttgactggaaaaaagatcacagcccacctaaagaggttgtggaaaatgctggacccaagacaaggcttggctgttctaaggaaagtcaagagagtggtggccagtttgatgagaggattgtcctcaaggaaacgccgttcccatgatgttctgactgtgcaattcctaattttgggcatgctgggcatgacaatcgcagctacggttcgcaaggaaagagacggcagtacggtcatacgcgcggaaggtaaggatgccgctacccaagtgagagtggaaaatggtacctgcgtcattctggccaccgacatgggctcttggtgtgatgatagcctttcttatgagtgcgtaaccatagatcaaggtgaggaacctgttgacgttgattgcttctgccgaaacgtggatggggtgtatctcgaatatggacggtgtggtaaacaagaaggaagcagaaccagacgctcagtgcttataccctcccacgctcaaggagagctgaccggacggggacataaatggttggagggcgactcactccgaacacatttgacccgcgtcgagggctgggtctggaaaaatcggctgttggccctcgctatggtgacagtcgtttggctcacgctggagtctgtggttactcgcgtggcagtgctggtggtgctcctctgtcttgcccctgtctacgcgtccaggtgtactcatttggaaaacagagattttgtcaccggcacccaggggacgactcgggtaaccctggtgcttgaactgggtggttgcgttactattaccgctgagggcaaaccctctatggatgtgtggctggatgcaatctatcaggagaatcccgcacaaaccagggaatattgccttcacgcaaagctgtccgatacaaaggtcgcggctaggtgcccaacaatgggaccggccaccctggcggaggaacatcagggaggtacagtgtgcaaacgggaccagagtgatagaggctggggtaatcactgcggcctgttcggcaaaggaagtattgtcgcttgcgtcaaggcagcctgtgaggccaaaaagaaggctactgggcacgtctatgacgccaacaagatcgtttatacagtgaaagtggaaccacacacaggggattacgtggcggccaacgagactcattccggtcgcaaaacggccagcttcaccgtgtcatccgaaaagaccatcctcactatgggggagtatggcgacgtttctctgctctgccgggtggctagcggagtcgacctggcccagacagtcatcctggaactggataaaacagttgagcatctgcctaccgcttggcaggtgcacagggattggtttaacgaccttgccctgccatggaaacatgaaggagcgagaaactggaataatgcagagcgactcgtagaattcggtgcccctcatgccgtgaagatggacgtctacaatctgggtgatcagaccggcgttctccttaaagctctcgctggcgtaccagttgcccacatcgaaggaacgaagtaccacctgaagtcaggccatgtaacttgcgaggtgggcctggagaagttgaaaatgaaaggtcttacgtacacaatgtgtgacaagaccaagttcacatggaagagggcccccacagatagcggccacgatactgtggtgatggaggtgaccttttctggaacaaaaccctgcagaatacccgtgcgggctgtagctcacggatctcccgatgtcaatgttgctatgctgattacacctaaccctaccatcgagaataacggtggtggttttattgagatgcagcttccgccaggcgataacatcatctacgtgggcgaactctcttaccagtggtttcagaaagggagttcaattgggcgggtcttccaaaaaacgaagaagggaatcgaacgattgacggttatcggcgagcacgcatgggattttggttccgcagggggattcctgtcttctattggtaaggcactgcataccgtgctggggggcgcattcaattctattttcgggggcgtggggttcctgcctaaactcctgctgggagtagccctggcctggttgggactgaatatgcggaatccgacgatgtccatgtcattcctcttggccggcgtgcttgtactggccatgacactgggcgttggcgccgatcaaggatgcgccatcaactttggcaagagagagctc

SEQ ID NO: 42 - DNA, 2496 bp, Artificial Sequence
  Synthetic Construct
tcatttaggacacacgattaactccacgtaaccagacgtttagctcaacgatccgttatttgtgtaaacctaattaaaattagcaagcaactcgctaatcgtctcttgactggtcttgtacagaccagcatttcgagtccctttttgggacccgcagttataccatgctgctcctcaagcgaggaacagtttgttttattttgttttttgttttgtttaacctttgtctggacctggaagttctccacaagttcctaaatagaaaaagaaaaacaagttgtaaaactgaccttttttctagtgtcgggtggatttctccaacaccttttacgacctgggttctgttccgaaccgacaagattcctttcagttctctcaccaccggtcaaactactctcctaacaggagttcctttgcggcaagggtactacaagactgacacgttaaggattaaaacccgtacgacccgtactgttagcgtcgatgccaagcgttcctttctctgccgtcatgccagtatgcgcgccttccattcctacggcgatgggttcactctcaccttttaccatggacgcagtaagaccggtggctgtacccgagaaccacactactatcggaaagaatactcacgcattggtatctagttccactccttggacaactgcaactaacgaagacggctttgcacctaccccacatagagcttatacctgccacaccatttgttcttccttcgtcttggtctgcgagtcacgaatatgggagggtgcgagttcctctcgactggcctgcccctgtatttaccaacctcccgctgagtgaggcttgtgtaaactgggcgcagctcccgacccagacctttttagccgacaaccgggagcgataccactgtcagcaaaccgagtgcgacctcagacaccaatgagcgcaccgtcacgaccaccacgaggagacagaacggggacagatgcgcaggtccacatgagtaaaccttttgtctctaaaacagtggccgtgggtcccctgctgagcccattgggaccacgaacttgacccaccaacgcaatgataatggcgactcccgtttgggagatacctacacaccgacctacgttagatagtcctcttagggcgtgtttggtcccttataacggaagtgcgtttcgacaggctatgtttccagcgccgatccacgggttgttaccctggccggtgggaccgcctccttgtagtccctccatgtcacacgtttgccctggtctcactatctccgaccccattagtgacgccggacaagccgtttccttcataacagcgaacgcagttccgtcggacactccggtttttcttccgatgacccgtgcagatactgcggttgttctagcaaatatgtcactttcaccttggtgtgtgtcccctaatgcaccgccggttgctctgagtaaggccagcgttttgccggtcgaagtggcacagtaggcttttctggtaggagtgataccccctcataccgctgcaaagagacgagacggcccaccgatcgcctcagctggaccgggtctgtcagtaggaccttgacctattttgtcaactcgtagacggatggcgaaccgtccacgtgtccctaaccaaattgctggaacgggacggtacctttgtacttcctcgctctttgaccttattacgtctcgctgagcatcttaagccacggggagtacggcacttctacctgcagatgttagacccactagtctggccgcaagaggaatttcgagagcgaccgcatggtcaacgggtgtagcttccttgcttcatggtggacttcagtccggtacattgaacgctccacccggacctcttcaacttttactttccagaatgcatgtgttacacactgttctggttcaagtgtaccttctcccgggggtgtctatcgccggtgctatgacaccactacctccactggaaaagaccttgttttgggacgtcttatgggcacgcccgacatcgagtgcctagagggctacagttacaacgatacgactaatgtggattgggatggtagctcttattgccaccaccaaaataactctacgtcgaaggcggtccgctattgtagtagatgcacccgcttgagagaatggtcaccaaagtctttccctcaagttaacccgcccagaaggttttttgcttcttcccttagcttgctaactgccaatagccgctcgtgcgtaccctaaaaccaaggcgtccccctaaggacagaagataaccattccgtgacgtatggcacgaccccccgcgtaagttaagataaaagcccccgcaccccaaggacggatttgaggacgaccctcatcgggaccggaccaaccctgacttatacgccttaggctgctatacagtaaggagaaccggccgcacgaacatgaccggtactgtgacccgcaaccgcggctagttcctacgcggtagttgaaaccgttctctctcgag

SEQ ID NO: 43 - protein, 793 aa, Artificial Sequence
  Synthetic Construct
MSGRKAQGKTLGVNMVRRGVRSLSNKIKQKTKQIGNRPGPSRGVQGFIFFFLFNILTGKKITAHLKRLWKMLDPRQGLAVLRKVKRVVASLMRGLSSRKRRSHDVLTVQFLILGMLACVGAATVRKERDGSTVIRAEGKDAATQVRVENGTCVILATDMGSWCDDSLSYECVTIDQGEEPVDVDCFCRNVDGVYLEYGRCGKQEGSRTRRSVLIPSHAQGELTGRGHKWLEGDSLRTHLTRVEGWVWKNRLLALAMVTVVWLTLESVVTRVAVLVVLLCLAPVYASRCTHLENRDFVTGTQGTTRVTLVLELGGCVTITAEGKPSMDVWLDAIYQENPAQTREYCLHAKLSDTKVAARCPTMGPATLAEEHQGGTVCKRDQSDRGWGNHCGLFGKGSIVACVKAACEAKKKATGHVYDANKIVYTVKEPHTGDYVAANETHSGRKTASFTVSSEKTILTMGEYGDVSLLCRVASGVDLAQTVILELDKTVEHLPTAWQVHRDWFNDLALPWKHEGARNWNNAERLVEFGAPHAVKMDVYNLGDQTGVLLKALAGVPVAHIEGTKYHLKSGHVTCEVGLEKLKMKGLTYTMCDKTKFTWKRAPTDSGHDTVVMEVTFSGTKPCRIPVRAVAHGSPDVNVAMLITPNPTIENNGGGFIEMQLPPGDNIIYVGELSYQWFQKGSSIGRVFQKTKKGIERLTVIGEHAWDFGSAGGFLSSIGKALHTVLGGAFNSIFGGVGFLPKLLLGVALAWLGLNMRNPTMSMSFLLAGVLVLAMTLGVGADQGCAINFGKREL

SEQ ID NO: 44 - DNA, 2500 bp, Artificial Sequence
  Synthetic Construct
agtaaatcctgtgtgctaattgaggtgcattggtctgcaaatcgagttgctaggcaataaacacatttggattaattttaatcgttcgttgagcgattagcagagaactgaccagaacatgtctggtcgtaaagctcagggaaaaaccctgggcgtcaatatggtacgacgaggagttcgctccttgtcaaacaaaataaaacaaaaaacaaaacaaattggaaacagacctggaccttcaagaggtgttcaaggatttatctttttctttttgttcaacattttgactggaaaaaagatcacagcccacctaaagaggttgtggaaaatgctggacccaagacaaggcttggctgttctaaggaaagtcaagagagtggtggccagtttgatgagaggattgtcctcaaggaaacgccgttcccatgatgttctgactgtgcaattcctaattttgggcatgctggcttgtgtcggagcagctaccgtgcgaaaagaacgcgacggaagcaccgtgataagggctgagggtaaggatgcggctacgcaggtgagagtagagaatggcacttgcgtaatactcgcgactgatatgggatcctggtgtgacgatagcctcagttatgaatgcgtaacaatagaccagggcgaagaacctgtggacgttgactgtttctgtagaaatgtggatggcgtttatctggagtacggccgctgtggaaaacaggagggctcacgaactcgaagatctgtgctgattccaagtcacgcgcaaggagagttgaccggtagaggccacaagtggcttgaaggggactcattgaggacccacctgactagggtggagggttgggtttggaagaatcggttgctcgcgctcgctatggtcaccgtcgtgtggctgacactggagagtgtcgtgactcgggttgctgtgttggttgtcctcctctgtttggccccagtgtacgcgtccaggtgtactcatttggaaaacagagattttgtcaccggcacccaggggacgactcgggtaaccctggtgcttgaactgggtggttgcgttactattaccgctgagggcaaaccctctatggatgtgtggctggatgcaatctatcaggagaatcccgcacaaaccagggaatattgccttcacgcaaagctgtccgatacaaaggtcgcggctaggtgcccaacaatgggaccggccaccctggcggaggaacatcagggaggtacagtgtgcaaacgggaccagagtgatagaggctggggtaatcactgcggcctgttcggcaaaggaagtattgtcgcttgcgtcaaggcagcctgtgaggccaaaaagaaggctactgggcacgtctatgacgccaacaagatcgtttatacagtgaaagtggaaccacacacaggggattacgtggcggccaacgagactcattccggtcgcaaaacggccagcttcaccgtgtcatccgaaaagaccatcctcactatgggggagtatggcgacgtttctctgctctgccgggtggctagcggagtcgacctggcccagacagtcatcctggaactggataaaacagttgagcatctgcctaccgcttggcaggtgcacagggattggtttaacgaccttgccctgccatggaaacatgaaggagcgagaaactggaataatgcagagcgactcgtagaattcggtgcccctcatgccgtgaagatggacgtctacaatctgggtgatcagaccggcgttctccttaaagctctcgctggcgtaccagttgcccacatcgaaggaacgaagtaccacctgaagtcaggccatgtaacttgcgaggtgggcctggagaagttgaaaatgaaaggtcttacgtacacaatgtgtgacaagaccaagttcacatggaagagggcccccacagatagcggccacgatactgtggtgatggaggtgaccttttctggaacaaaaccctgcagaatacccgtgcgggctgtagctcacggatctcccgatgtcaatgttgctatgctgattacacctaaccctaccatcgagaataacggtggtggttttattgagatgcagcttccgccaggcgataacatcatctacgtgggcgaactctcttaccagtggtttcagaaagggagttcaattgggcgggtcttccaaaaaacgaagaagggaatcgaacgattgacggttatcggcgagcacgcatgggattttggttccgcagggggattcctgtcttctattggtaaggcactgcataccgtgctggggggcgcattcaattctattttcgggggcgtggggttcctgcctaaactcctgctgggagtagccctggcctggttgggactgaatatgcggaatccgacgatgtccatgtcattcctcttggccggcgtgcttgtactggccatgacactgggcgttggcgccgatcaaggatgcgccatcaactttggcaagagagagctc

SEQ ID NO: 45 - DNA, 2500 bp, Artificial Sequence
  Synthetic Construct
tcatttaggacacacgattaactccacgtaaccagacgtttagctcaacgatccgttatttgtgtaaacctaattaaaattagcaagcaactcgctaatcgtctcttgactggtcttgtacagaccagcatttcgagtccctttttgggacccgcagttataccatgctgctcctcaagcgaggaacagtttgttttattttgttttttgttttgtttaacctttgtctggacctggaagttctccacaagttcctaaatagaaaaagaaaaacaagttgtaaaactgaccttttttctagtgtcgggtggatttctccaacaccttttacgacctgggttctgttccgaaccgacaagattcctttcagttctctcaccaccggtcaaactactctcctaacaggagttcctttgcggcaagggtactacaagactgacacgttaaggattaaaacccgtacgaccgaacacagcctcgtcgatggcacgcttttcttgcgctgccttcgtggcactattcccgactcccattcctacgccgatgcgtccactctcatctcttaccgtgaacgcattatgagcgctgactataccctaggaccacactgctatcggagtcaatacttacgcattgttatctggtcccgcttcttggacacctgcaactgacaaagacatctttacacctaccgcaaatagacctcatgccggcgacaccttttgtcctcccgagtgcttgagcttctagacacgactaaggttcagtgcgcgttcctctcaactggccatctccggtgttcaccgaacttcccctgagtaactcctgggtggactgatcccacctcccaacccaaaccttcttagccaacgagcgcgagcgataccagtggcagcacaccgactgtgacctctcacagcactgagcccaacgacacaaccaacaggaggagacaaaccggggtcacatgcgcaggtccacatgagtaaaccttttgtctctaaaacagtggccgtgggtcccctgctgagcccattgggaccacgaacttgacccaccaacgcaatgataatggcgactcccgtttgggagatacctacacaccgacctacgttagatagtcctcttagggcgtgtttggtcccttataacggaagtgcgtttcgacaggctatgtttccagcgccgatccacgggttgttaccctggccggtgggaccgcctccttgtagtccctccatgtcacacgtttgccctggtctcactatctccgaccccattagtgacgccggacaagccgtttccttcataacagcgaacgcagttccgtcggacactccggtttttcttccgatgacccgtgcagatactgcggttgttctagcaaatatgtcactttcaccttggtgtgtgtcccctaatgcaccgccggttgctctgagtaaggccagcgttttgccggtcgaagtggcacagtaggcttttctggtaggagtgataccccctcataccgctgcaaagagacgagacggcccaccgatcgcctcagctggaccgggtctgtcagtaggaccttgacctattttgtcaactcgtagacggatggcgaaccgtccacgtgtccctaaccaaattgctggaacgggacggtacctttgtacttcctcgctctttgaccttattacgtctcgctgagcatcttaagccacggggagtacggcacttctacctgcagatgttagacccactagtctggccgcaagaggaatttcgagagcgaccgcatggtcaacgggtgtagcttccttgcttcatggtggacttcagtccggtacattgaacgctccacccggacctcttcaacttttactttccagaatgcatgtgttacacactgttctggttcaagtgtaccttctcccgggggtgtctatcgccggtgctatgacaccactacctccactggaaaagaccttgttttgggacgtcttatgggcacgcccgacatcgagtgcctagagggctacagttacaacgatacgactaatgtggattgggatggtagctcttattgccaccaccaaaataactctacgtcgaaggcggtccgctattgtagtagatgcacccgcttgagagaatggtcaccaaagtctttccctcaagttaacccgcccagaaggttttttgcttcttcccttagcttgctaactgccaatagccgctcgtgcgtaccctaaaaccaaggcgtccccctaaggacagaagataaccattccgtgacgtatggcacgaccccccgcgtaagttaagataaaagcccccgcaccccaaggacggatttgaggacgaccctcatcgggaccggaccaaccctgacttatacgccttaggctgctacaggtacagtaaggagaaccggccgcacgaacatgaccggtactgtgacccgcaaccgcggctagttcctacgcggtagttgaaaccgttctctctcgag

SEQ ID NO: 46 - protein, 794 aa, Artificial Sequence
  Synthetic Construct
MSGRKAQGKTLGVNMVRRGVRSLSNKIKQKTKQIGNRPGPSRGVQGFIFFFLFNILTGKKITAHLKRLWKMLDPRQGLAVLRKVKRVVASLMRGLSSRKRRSHDVLTVQFLILGMLGMTIAATVRRERDGSMVIRAEGRDAATQVRVENGTCVILATDMGSWCDDSLAYECVTIDQGEEPVDVDCFCRGVEKVTLEYGRCGRREGSRSRRSVLIPSHAQRDLTGRGHQWLEGEAVKAHLTRVEGWVWKNKLFTLSLVMVAWLMVDGLLPRILIVVVALALAPAYASRCTHLENRDFVTGVQGTTRLTLVLELGGCVTVTADGKPSLDVWLDSIYQESPAQTREYCLHAKLTGTKVAARCPTMGPATLPEEHQSGTVCKRDQSDRGWGNHCGLFGKGSIVTCVKVTCEDKKKATGHVYDVNKITYTIKVEPHTGEFVAANETHSGRKSASFTVSSEKTILTLGDYGDVSLLCRVASGVDLAQTVVLALDKTHEHLPTAWQVHRDWFNDLALPWKHDGAEAWNEAGRLVEFGTPHAVKMDVFNLGDQTGVLLKSLAGVPVASIEGTKYHLKSGHVTCEVGLEKLKMKGLTYTVCDKTKFTWKRAPTDSGHDTVVMEVGFSGTRPCRIPVRAVAHGVPEVNVAMLITPNPTMENNGGGFIEMQLPPGDNIIYVGDLDHQWFQKGSSIGRVLQKTRKGIERLTVLGEHAWDFGSVGGVMTSIGRAMHTVLGGAFNTLLGGVGFLPKILLGVAMAWLGLNMRNPTLSMGFLLSGGLVLAMTLGVGADQGCAINFGKREL

SEQ ID NO: 47 - DNA, 2500 bp, Artificial Sequence
  Synthetic Construct
agtaaatcctgtgtgctaattgaggtgcattggtctgcaaatcgagttgctaggcaataaacacatttggattaattttaatcgttcgttgagcgattagcagagaactgaccagaacatgtctggtcgtaaagctcagggaaaaaccctgggcgtcaatatggtacgacgaggagttcgctccttgtcaaacaaaataaaacaaaaaacaaaacaaattggaaacagacctggaccttcaagaggtgttcaaggatttatctttttctttttgttcaacattttgactggaaaaaagatcacagcccacctaaagaggttgtggaaaatgctggacccaagacaaggcttggctgttctaaggaaagtcaagagagtggtggccagtttgatgagaggattgtcctcaaggaaacgccgttcccatgatgttctgactgtgcaattcctaattttgggcatgctggggatgacgatcgcagctactgtgcgaagggagagagacggctctatggtgatcagagccgaaggtagggacgctgcgacccaggtgagggtcgaaaatggcacctgtgttattctggcgaccgacatgggctcctggtgtgatgattctctggcttatgaatgtgttactattgatcagggtgaagagcctgtggacgtggactgtttctgtagaggcgtcgagaaagtgaccctggaatatggacgatgtggccggcgagaaggctccaggagtcggagatccgtgttgatcccttcacatgcgcagcgcgatctgacagggaggggtcaccagtggctcgaaggcgaagcagtcaaggcccatctgactcgcgttgaaggctgggtgtggaaaaacaaactctttacccttagcctggtgatggtcgcgtggctgatggtagacggactccttccccgcattctcattgttgtggtggctctcgcgctcgcccctgcatacgcgtccaggtgtacgcacctcgaaaatcgagatttcgtcacaggcgtccaaggtactacccggctcaccctcgtgctggagctgggaggctgtgtcactgttacagccgacggaaaacctagtctggatgtgtggctggactccatctatcaggagagcccggcacagaccagggagtactgcctccacgctaagctgactgggacaaaggtagccgcaagatgtcccacaatggggcctgccaccttgcccgaggaacaccaatccggtacggtatgcaagcgagatcagtctgatcgcggatgggggaatcattgcggcctcttcggtaaaggcagcattgtcacttgcgtgaaggtgacatgcgaggacaagaagaaggccacaggtcatgtatatgatgtgaacaaaatcacatataccattaaggtagaaccacatacaggggaattcgtggcagcaaacgagactcatagcggacgaaagtccgcctccttcaccgtctcctccgagaaaacaatcctgaccctcggagactacggcgacgtatctttgctgtgcagggtggccagcggcgtggaccttgctcagacagtcgtgttggccctggacaagacacatgagcacttgccaacagcctggcaggtgcacagggactggtttaacgacctggcgctcccgtggaaacatgacggcgctgaagcatggaatgaggcagggagactggtggaatttggaaccccacacgccgtaaagatggacgttttcaatcttggtgaccagacaggggtgctcctgaaatcactggcgggcgtgcctgtagccagcatcgagggcacaaagtatcacctgaagtctgggcatgtaacctgcgaagtgggcctggaaaagctgaagatgaaaggacttacgtacactgtttgtgataagaccaagtttacatggaagcgagccccaacggattccggccatgataccgtcgtgatggaggttggtttctccggcaccagaccatgtagaataccagtgagagctgtcgcccacggtgtacccgaggtaaacgtggccatgctgattacaccgaatcccactatggagaacaatggcggagggttcatcgaaatgcagctgccgcctggagacaacatcatttatgtcggcgacctcgatcatcaatggttccagaaagggtcttccatcggccgcgtccttcagaagacacgaaaaggcattgaaagacttacagtcctgggcgaacatgcctgggacttcgggtcagttggcggggtaatgacaagcataggcagagctatgcacaccgttctcggtggggcatttaatactctgttgggtggcgtgggttttcttccgaaaatcctgctcggtgtcgcaatggcctggcttggactgaatatgcgcaatcctacactgagtatggggtttcttctgtcaggaggcctggtcctggcaatgactctgggagtgggcgccgatcaaggatgcgccatcaactttggcaagagagagctc

SEQ ID NO: 48 - DNA, 2500 bp, Artificial Sequence
  Synthetic Construct
tcatttaggacacacgattaactccacgtaaccagacgtttagctcaacgatccgttatttgtgtaaacctaattaaaattagcaagcaactcgctaatcgtctcttgactggtcttgtacagaccagcatttcgagtccctttttgggacccgcagttataccatgctgctcctcaagcgaggaacagtttgttttattttgttttttgttttgtttaacctttgtctggacctggaagttctccacaagttcctaaatagaaaaagaaaaacaagttgtaaaactgaccttttttctagtgtcgggtggatttctccaacaccttttacgacctgggttctgttccgaaccgacaagattcctttcagttctctcaccaccggtcaaactactctcctaacaggagttcctttgcggcaagggtactacaagactgacacgttaaggattaaaacccgtacgacccctactgctagcgtcgatgacacgcttccctctctctgccgagataccactagtctcggcttccatccctgcgacgctgggtccactcccagcttttaccgtggacacaataagaccgctggctgtacccgaggaccacactactaagagaccgaatacttacacaatgataactagtcccacttctcggacacctgcacctgacaaagacatctccgcagctctttcactgggaccttatacctgctacaccggccgctcttccgaggtcctcagcctctaggcacaactagggaagtgtacgcgtcgcgctagactgtccctccccagtggtcaccgagcttccgcttcgtcagttccgggtagactgagcgcaacttccgacccacacctttttgtttgagaaatgggaatcggaccactaccagcgcaccgactaccatctgcctgaggaaggggcgtaagagtaacaacaccaccgagagcgcgagcggggacgtatgcgcaggtccacatgcgtggagcttttagctctaaagcagtgtccgcaggttccatgatgggccgagtgggagcacgacctcgaccctccgacacagtgacaatgtcggctgccttttggatcagacctacacaccgacctgaggtagatagtcctctcgggccgtgtctggtccctcatgacggaggtgcgattcgactgaccctgtttccatcggcgttctacagggtgttaccccggacggtggaacgggctccttgtggttaggccatgccatacgttcgctctagtcagactagcgcctacccccttagtaacgccggagaagccatttccgtcgtaacagtgaacgcacttccactgtacgctcctgttcttcttccggtgtccagtacatatactacacttgttttagtgtatatggtaattccatcttggtgtatgtccccttaagcaccgtcgtttgctctgagtatcgcctgctttcaggcggaggaagtggcagaggaggctcttttgttaggactgggagcctctgatgccgctgcatagaaacgacacgtcccaccggtcgccgcacctggaacgagtctgtcagcacaaccgggacctgttctgtgtactcgtgaacggttgtcggaccgtccacgtgtccctgaccaaattgctggaccgcgagggcacctttgtactgccgcgacttcgtaccttactccgtccctctgaccaccttaaaccttggggtgtgcggcatttctacctgcaaaagttagaaccactggtctgtccccacgaggactttagtgaccgcccgcacggacatcggtcgtagctcccgtgtttcatagtggacttcagacccgtacattggacgcttcacccggaccttttcgacttctactttcctgaatgcatgtgacaaacactattctggttcaaatgtaccttcgctcggggttgcctaaggccggtactatggcagcactacctccaaccaaagaggccgtggtctggtacatcttatggtcactctcgacagcgggtgccacatgggctccatttgcaccggtacgactaatgtggcttagggtgatacctcttgttaccgcctcccaagtagctttacgtcgacggcggacctctgttgtagtaaatacagccgctggagctagtagttaccaaggtctttcccagaaggtagccggcgcaggaagtcttctgtgcttttccgtaactttctgaatgtcaggacccgcttgtacggaccctgaagcccagtcaaccgccccattactgttcgtatccgtctcgatacgtgtggcaagagccaccccgtaaattatgagacaacccaccgcacccaaaagaaggcttttaggacgagccacagcgttaccggaccgaacctgacttatacgcgttaggatgtgactcataccccaaagaagacagtcctccggaccaggaccgttactgagaccctcacccgcggctagttcctacgcggtagttgaaaccgttctctctcgag

SEQ ID NO: 49 - protein, 791 aa, Artificial Sequence
  Synthetic Construct
MSGRKAQGKTLGVNMVRRGVRSLSNKIKQKTKQIGNRPGGVQGFIFFFLFNILTGKKITAHLKRLWKMLDPRQGLAVLRKVKRVVASLMRGLSSRKRRSHDVLTVQFLILGMLGMTIAATVRKERDGSTVIRAEGKDAATQVRVENGTCVILATDMGSWCDDSLSYECVTIDQGEEPVDVDCFCRNVDGVYLEYGRCGKQEGSRTRRSVLIPSHAQGELTGRGHKWLEGDSLRTHLTRVEGWVWKNRLLALAMVTVVWLTLESVVTRVAVLVVLLCLAPVYASRCTHLENRDFVTGTQGTTRVTLVLELGGCVTITAEGKPSMDVWLDAIYQENPAQTREYCLHAKLSDTKVAARCPTMGPATLAEEHQGGTVCKRDQSDRGWGNHCGLFGKGSIVACVKAACEAKKKATGHVYDANKIVYTVKVEPHTGDYVAANETHSGRKTASFTVSSEKTILTMGEYGDVSLLCRVASGVDLAQTVILELDKTVEHLPTAWQVHRDWFNDLALPWKHEGARNWNNAERLVEFGAPHAVKMDVYNLGDQTGVLLKALAGVPVAHIEGTKYHLKSGHVTCEVGLEKLKMKGLTYTMCDKTKFTWKRAPTDSGHDTVVMEVTFSGTKPCRIPVRAVAHGSPDVNVAMLITPNPTIENNGGGFIEMQLPPGDNIIYVGELSYQWFQKGSSIGRVFQKTKKGIERLTVIGEHAWDFGSAGGFLSSIGKALHTVLGGAFNSIFGGVGFLPKLLLGVALAWLGLNMRNPTMSMSFLLAGVLVLAMTLGVGADQGCAINFGKREL

SEQ ID NO: 50 - DNA, 2491 bp, Artificial Sequence
  Synthetic Construct
agtaaatcctgtgtgctaattgaggtgcattggtctgcaaatcgagttgctaggcaataaacacatttggattaattttaatcgttcgttgagcgattagcagagaactgaccagaacatgtctggtcgtaaagctcagggaaaaaccctgggcgtcaatatggtacgacgaggagttcgctccttgtcaaacaaaataaaacaaaaaacaaaacaaattggaaacagacctggaggtgttcaaggatttatctttttctttttgttcaacattttgactggaaaaaagatcacagcccacctaaagaggttgtggaaaatgctggacccaagacaaggcttggctgttctaaggaaagtcaagagagtggtggccagtttgatgagaggattgtcctcaaggaaacgccgttcccatgatgttctgactgtgcaattcctaattttgggcatgctgggcatgacaatcgcagctacggttcgcaaggaaagagacggcagtacggtcatacgcgcggaaggtaaggatgccgctacccaagtgagagtggaaaatggtacctgcgtcattctggccaccgacatgggctcttggtgtgatgatagcctttcttatgagtgcgtaaccatagatcaaggtgaggaacctgttgacgttgattgcttctgccgaaacgtggatggggtgtatctcgaatatggacggtgtggtaaacaagaaggaagcagaaccagacgctcagtgcttataccctcccacgctcaaggagagctgaccggacggggacataaatggttggagggcgactcactccgaacacatttgacccgcgtcgagggctgggtctggaaaaatcggctgttggccctcgctatggtgacagtcgtttggctcacgctggagtctgtggttactcgcgtggcagtgctggtggtgctcctctgtcttgcccctgtctacgcgtccaggtgtactcatttggaaaacagagattttgtcaccggcacccaggggacgactcgggtaaccctggtgcttgaactgggtggttgcgttactattaccgctgagggcaaaccctctatggatgtgtggctggatgcaatctatcaggagaatcccgcacaaaccagggaatattgccttcacgcaaagctgtccgatacaaaggtcgcggctaggtgcccaacaatgggaccggccaccctggcggaggaacatcagggaggtacagtgtgcaaacgggaccagagtgatagaggctggggtaatcactgcggcctgttcggcaaaggaagtattgtcgcttgcgtcaaggcagcctgtgaggccaaaaagaaggctactgggcacgtctatgacgccaacaagatcgtttatacagtgaaagtggaaccacacacaggggattacgtggcggccaacgagactcattccggtcgcaaaacggccagcttcaccgtgtcatccgaaaagaccatcctcactatgggggagtatggcgacgtttctctgctctgccgggtggctagcggagtcgacctggcccagacagtcatcctggaactggataaaacagttgagcatctgcctaccgcttggcaggtgcacagggattggtttaacgaccttgccctgccatggaaacatgaaggagcgagaaactggaataatgcagagcgactcgtagaattcggtgcccctcatgccgtgaagatggacgtctacaatctgggtgatcagaccggcgttctccttaaagctctcgctggcgtaccagttgcccacatcgaaggaacgaagtaccacctgaagtcaggccatgtaacttgcgaggtgggcctggagaagttgaaaatgaaaggtcttacgtacacaatgtgtgacaagaccaagttcacatggaagagggcccccacagatagcggccacgatactgtggtgatggaggtgaccttttctggaacaaaaccctgcagaatacccgtgcgggctgtagctcacggatctcccgatgtcaatgttgctatgctgattacacctaaccctaccatcgagaataacggtggtggttttattgagatgcagcttccgccaggcgataacatcatctacgtgggcgaactctcttaccagtggtttcagaaagggagttcaattgggcgggtcttccaaaaaacgaagaagggaatcgaacgattgacggttatcggcgagcacgcatgggattttggttccgcagggggattcctgtcttctattggtaaggcactgcataccgtgctggggggcgcattcaattctattttcgggggcgtggggttcctgcctaaactcctgctgggagtagccctggcctggttgggactgaatatgcggaatccgacgatgtccatgtcattcctcttggccggcgtgcttgtactggccatgacactgggcgttggcgccgatcaaggatgcgccatcaactttggcaagagagagctc

SEQ ID NO: 51 - DNA, 2491 bp, Artificial Sequence
  Synthetic Construct
tcatttaggacacacgattaactccacgtaaccagacgtttagctcaacgatccgttatttgtgtaaacctaattaaaattagcaagcaactcgctaatcgtctcttgactggtcttgtacagaccagcatttcgagtccctttttgggacccgcagttataccatgctgctcctcaagcgaggaacagtttgttttattttgttttttgttttgtttaacctttgtctggacctccacaagttcctaaatagaaaaagaaaaacaagttgtaaaactgaccttttttctagtgtcgggtggatttctccaacaccttttacgacctgggttctgttccgaaccgacaagattcctttcagttctctcaccaccggtcaaactactctcctaacaggagttcctttgcggcaagggtactacaagactgacacgttaaggattaaaacccgtacgacccgtactgttagcgtcgatgccaagcgttcctttctctgccgtcatgccagtatgcgcgccttccattcctacggcgatgggttcactctcaccttttaccatggacgcagtaagaccggtggctgtacccgagaaccacactactatcggaaagaatactcacgcattggtatctagttccactccttggacaactgcaactaacgaagacggctttgcacctaccccacatagagcttatacctgccacaccatttgttcttccttcgtcttggtctgcgagtcacgaatatgggagggtgcgagttcctctcgactggcctgcccctgtatttaccaacctcccgctgagtgaggcttgtgtaaactgggcgcagctcccgacccagacctttttagccgacaaccgggagcgataccactgtcagcaaaccgagtgcgacctcagacaccaatgagcgcaccgtcacgaccaccacgaggagacagaacggggacagatgcgcaggtccacatgagtaaaccttttgtctctaaaacagtggccgtgggtcccctgctgagcccattgggaccacgaacttgacccaccaacgcaatgataatggcgactcccgtttgggagatacctacacaccgacctacgttagatagtcctcttagggcgtgtttggtcccttataacggaagtgcgtttcgacaggctatgtttccagcgccgatccacgggttgttaccctggccggtgggaccgcctccttgtagtccctccatgtcacacgtttgccctggtctcactatctccgaccccattagtgacgccggacaagccgtttccttcataacagcgaacgcagttccgtcggacactccggtttttcttccgatgacccgtgcagatactgcggttgttctagcaaatatgtcactttcaccttggtgtgtgtcccctaatgcaccgccggttgctctgagtaaggccagcgttttgccggtcgaagtggcacagtaggcttttctggtaggagtgataccccctcataccgctgcaaagagacgagacggcccaccgatcgcctcagctggaccgggtctgtcagtaggaccttgacctattttgtcaactcgtagacggatggcgaaccgtccacgtgtccctaaccaaattgctggaacgggacggtacctttgtacttcctcgctctttgaccttattacgtctcgctgagcatcttaagccacggggagtacggcacttctacctgcagatgttagacccactagtctggccgcaagaggaatttcgagagcgaccgcatggtcaacgggtgtagcttccttgcttcatggtggacttcagtccggtacattgaacgctccacccggacctcttcaacttttactttccagaatgcatgtgttacacactgttctggttcaagtgtaccttctcccgggggtgtctatcgccggtgctatgacaccactacctccactggaaaagaccttgttttgggacgtcttatgggcacgcccgacatcgagtgcctagagggctacagttacaacgatacgactaatgtggattgggatggtagctcttattgccaccaccaaaataactctacgtcgaaggcggtccgctattgtagtagatgcacccgcttgagagaatggtcaccaaagtctttccctcaagttaacccgcccagaaggttttttgcttcttcccttagcttgctaactgccaatagccgctcgtgcgtaccctaaaaccaaggcgtccccctaaggacagaagataaccattccgtgacgtatggcacgaccccccgcgtaagttaagataaaagcccccgcaccccaaggacggatttgaggacgaccctcatcgggaccggaccaaccctgacttatacgccttaggctgctacaggtacagtaaggagaaccggccgcacgaacatgaccggtactgtgacccgcaaccgcggctagttcctacgcggtagttgaaaccgttctctctcgag

SEQ ID NO: 52 - protein, 730 aa, Artificial Sequence
  Synthetic Construct
MSKKPGGPGKSRAVYLLKRGMPRVLSLIGLKRSSKQKKRGGTDWMSWLLVIGMLGMTIAATVRKERDGSTVIRAEGKDAATQVRVENGTCVILATDMGSWCDDSLSYECVTIDQGEEPVDVDCFCRNVDGVYLEYGRCGKQEGSRTRRSVLIPSHAQGELTGRGHKWLEGDSLRTHLTRVEGWVWKNRLLALAMVTVVWLTLESVVTRVAVLVVLLCLAPVYASRCTHLENRDFVTGTQGTTRVTLVLELGGCVTITAEGKPSMDVWLDAIYQENPAQTREYCLHAKLSDTKVAARCPTMGPATLAEEHQGGTVCKRDQSDRGWGNHCGLFGKGSIVACVKAACEAKKKATGHVYDANKIVYTVKVEPHTGDYVAANETHSGRKTASFTVSSEKTILTMGEYGDVSLLCRVASGVDLAQTVILELDKTVEHLPTAWQVHRDWFNDLALPWKHEGARNWNNAERLVEFGAPHAVKMDVYNLGDQTGVLLKALAGVPVAHIEGTKYHLKSGHVTCEVGLEKLKMKGLTYTMCDKTKFTWKRAPTDSGHDTVVMEVTFSGTKPCRIPVRAVAHGSPDVNVAMLITPNPTIENNGGGFIEMQLPPGDNIIYVGELSYQWFQKGSSIGRVFQKTKKGIERLTVIGEHAWDFGSAGGFLSSIGKALHTVLGGAFNSIFGGVGFLPKLLLGVALAWLGLNMRNPTMSMSFLLAGVLVLAMTLGVGADTGCAIDISRQ

SEQ ID NO: 53 - DNA, 2286 bp, Artificial Sequence
  Synthetic Construct
agtagttcgcctgtgtgagctgacaaacttagtagtgtttgtgaggattaacaacaattaacacagtgcgagctgtttcttagcacgaagatctcgatgtctaagaaaccaggagggcccggcaagagccgggctgtctatttgctaaaacgcggaatgccccgcgtgttgtccttgattggacttaagcggagctccaaacaaaagaaacgggggggaacagactggatgagctggctgctcgtaatcggcatgctgggcatgacaatcgcagctacggttcgcaaggaaagagacggcagtacggtcatacgcgcggaaggtaaggatgccgctacccaagtgagagtggaaaatggtacctgcgtcattctggccaccgacatgggctcttggtgtgatgatagcctttcttatgagtgcgtaaccatagatcaaggtgaggaacctgttgacgttgattgcttctgccgaaacgtggatggggtgtatctcgaatatggacggtgtggtaaacaagaaggaagcagaaccagacgctcagtgcttataccctcccacgctcaaggagagctgaccggacggggacataaatggttggagggcgactcactccgaacacatttgacccgcgtcgagggctgggtctggaaaaatcggctgttggccctcgctatggtgacagtcgtttggctcacgctggagtctgtggttactcgcgtggcagtgctggtggtgctcctctgtcttgcccctgtctacgcgtccaggtgtactcatttggaaaacagagattttgtcaccggcacccaggggacgactcgggtaaccctggtgcttgaactgggtggttgcgttactattaccgctgagggcaaaccctctatggatgtgtggctggatgcaatctatcaggagaatcccgcacaaaccagggaatattgccttcacgcaaagctgtccgatacaaaggtcgcggctaggtgcccaacaatgggaccggccaccctggcggaggaacatcagggaggtacagtgtgcaaacgggaccagagtgatagaggctggggtaatcactgcggcctgttcggcaaaggaagtattgtcgcttgcgtcaaggcagcctgtgaggccaaaaagaaggctactgggcacgtctatgacgccaacaagatcgtttatacagtgaaagtggaaccacacacaggggattacgtggcggccaacgagactcattccggtcgcaaaacggccagcttcaccgtgtcatccgaaaagaccatcctcactatgggggagtatggcgacgtttctctgctctgccgggtggctagcggagtcgacctggcccagacagtcatcctggaactggataaaacagttgagcatctgcctaccgcttggcaggtgcacagggattggtttaacgaccttgccctgccatggaaacatgaaggagcgagaaactggaataatgcagagcgactcgtagaattcggtgcccctcatgccgtgaagatggacgtctacaatctgggtgatcagaccggcgttctccttaaagctctcgctggcgtaccagttgcccacatcgaaggaacgaagtaccacctgaagtcaggccatgtaacttgcgaggtgggcctggagaagttgaaaatgaaaggtcttacgtacacaatgtgtgacaagaccaagttcacatggaagagggcccccacagatagcggccacgatactgtggtgatggaggtgaccttttctggaacaaaaccctgcagaatacccgtgcgggctgtagctcacggatctcccgatgtcaatgttgctatgctgattacacctaaccctaccatcgagaataacggtggtggttttattgagatgcagcttccgccaggcgataacatcatctacgtgggcgaactctcttaccagtggtttcagaaagggagttcaattgggcgggtcttccaaaaaacgaagaagggaatcgaacgattgacggttatcggcgagcacgcatgggattttggttccgcagggggattcctgtcttctattggtaaggcactgcataccgtgctggggggcgcattcaattctattttcgggggcgtggggttcctgcctaaactcctgctgggagtagccctggcctggttgggactgaatatgcggaatccgacgatgtccatgtcattcctcttggccggcgtgcttgtactggccatgacactgggcgttggcgccgacactgggtgtgccatagacatcagccggcaa

SEQ ID NO: 54 - DNA, 2286 bp, Artificial Sequence
  Synthetic Construct
tcatcaagcggacacactcgactgtttgaatcatcacaaacactcctaattgttgttaattgtgtcacgctcgacaaagaatcgtgcttctagagctacagattctttggtcctcccgggccgttctcggcccgacagataaacgattttgcgccttacggggcgcacaacaggaactaacctgaattcgcctcgaggtttgttttctttgcccccccttgtctgacctactcgaccgacgagcattagccgtacgacccgtactgttagcgtcgatgccaagcgttcctttctctgccgtcatgccagtatgcgcgccttccattcctacggcgatgggttcactctcaccttttaccatggacgcagtaagaccggtggctgtacccgagaaccacactactatcggaaagaatactcacgcattggtatctagttccactccttggacaactgcaactaacgaagacggctttgcacctaccccacatagagcttatacctgccacaccatttgttcttccttcgtcttggtctgcgagtcacgaatatgggagggtgcgagttcctctcgactggcctgcccctgtatttaccaacctcccgctgagtgaggcttgtgtaaactgggcgcagctcccgacccagacctttttagccgacaaccgggagcgataccactgtcagcaaaccgagtgcgacctcagacaccaatgagcgcaccgtcacgaccaccacgaggagacagaacggggacagatgcgcaggtccacatgagtaaaccttttgtctctaaaacagtggccgtgggtcccctgctgagcccattgggaccacgaacttgacccaccaacgcaatgataatggcgactcccgtttgggagatacctacacaccgacctacgttagatagtcctcttagggcgtgtttggtcccttataacggaagtgcgtttcgacaggctatgtttccagcgccgatccacgggttgttaccctggccggtgggaccgcctccttgtagtccctccatgtcacacgtttgccctggtctcactatctccgaccccattagtgacgccggacaagccgtttccttcataacagcgaacgcagttccgtcggacactccggtttttcttccgatgacccgtgcagatactgcggttgttctagcaaatatgtcactttcaccttggtgtgtgtcccctaatgcaccgccggttgctctgagtaaggccagcgttttgccggtcgaagtggcacagtaggcttttctggtaggagtgataccccctcataccgctgcaaagagacgagacggcccaccgatcgcctcagctggaccgggtctgtcagtaggaccttgacctattttgtcaactcgtagacggatggcgaaccgtccacgtgtccctaaccaaattgctggaacgggacggtacctttgtacttcctcgctctttgaccttattacgtctcgctgagcatcttaagccacggggagtacggcacttctacctgcagatgttagacccactagtctggccgcaagaggaatttcgagagcgaccgcatggtcaacgggtgtagcttccttgcttcatggtggacttcagtccggtacattgaacgctccacccggacctcttcaacttttactttccagaatgcatgtgttacacactgttctggttcaagtgtaccttctcccgggggtgtctatcgccggtgctatgacaccactacctccactggaaaagaccttgttttgggacgtcttatgggcacgcccgacatcgagtgcctagagggctacagttacaacgatacgactaatgtggattgggatggtagctcttattgccaccaccaaaataactctacgtcgaaggcggtccgctattgtagtagatgcacccgcttgagagaatggtcaccaaagtctttccctcaagttaacccgcccagaaggttttttgcttcttcccttagcttgctaactgccaatagccgctcgtgcgtaccctaaaaccaaggcgtccccctaaggacagaagataaccattccgtgacgtatggcacgaccccccgcgtaagttaagataaaagcccccgcaccccaaggacggatttgaggacgaccctcatcgggaccggaccaaccctgacttatacgccttaggctgctacaggtacagtaaggagaaccggccgcacgaacatgaccggtactgtgacccgcaaccgcggctgtgacccacacggtatctgtagtcggccgtt

SEQ ID NO: 55 - protein, 727 aa, Artificial Sequence
  Synthetic Construct
MSKKPGGPGKSRAVYLLKRGMPRVLSLIGLKRSSKQKKRGGKTGIAVMIGMLACVGAATVRKERDGSTVIRAEGKDAATQVRVENGTCVILATDMGSWCDDSLSYECVTIDQGEEPVDVDCFCRNVDGVYLEYGRCGKQEGSRTRRSVLIPSHAQGELTGRGHKWLEGDSLRTHLTRVEGWVWKNRLLALAMVTVVWLTLESVVTRVAVLVVLLCLAPVYASRCTHLENRDFVTGTQGTTRVTLVLELGGCVTITAEGKPSMDVWLDAIYQENPAQTREYCLHAKLSDTKVAARCPTMGPATLAEEHQGGTVCKRDQSDRGWGNHCGLFGKGSIVACVKAACEAKKKATGHVYDANKIVYTVKVEPHTGDYVAANETHSGRKTASFTVSSEKTILTMGEYGDVSLLCRVASGVDLAQTVILELDKTVEHLPTAWQVHRDWFNDLALPWKHEGARNWNNAERLVEFGAPAVKMDVYNLGDQTGVLLKALAGVPVAHIEGTKYHLKSGHVTCEVGLEKLKMKGLTYTMCDKTKFTWKRAPTDSGHDTVVMEVTFSGTKPCRIPVRAVAHGSPDVNVAMLITPNPTIENNGGGFIEMQLPPGDNIIYVGELSYQWFQKGSSIGRVFQKTKKGIERLTVIGEHAWDFGSAGGFLSSIGKALHTVLGGAFNSIFGGVGFLPKLLLGVALAWLGLNMRNPTMSMSFLLAGVLVLAMTLGVGADTGCAIDISRQ

SEQ ID NO: 56 - DNA, 2280 bp, Artificial Sequence
  Synthetic Construct
agtagttcgcctgtgtgagctgacaaacttagtagtgtttgtgaggattaacaacaattaacacagtgcgagctgtttcttagcacgaagatctcgatgtctaagaaaccaggagggcccggcaagagccgggctgtctatttgctaaaacgcggaatgccccgcgtgttgtccttgattggacttaagcggagctccaagcaaaagaaacgcgggggaaagacaggcatagctgtgatgataggcatgctggcttgtgtcggagcagctaccgtgcgaaaagaacgcgacggaagcaccgtgataagggctgagggtaaggatgcggctacgcaggtgagagtagagaatggcacttgcgtaatactcgcgactgatatgggatcctggtgtgacgatagcctcagttatgaatgcgtaacaatagaccagggcgaagaacctgtggacgttgactgtttctgtagaaatgtggatggcgtttatctggagtacggccgctgtggaaaacaggagggctcacgaactcgaagatctgtgctgattccaagtcacgcgcaaggagagttgaccggtagaggccacaagtggcttgaaggggactcattgaggacccacctgactagggtggagggttgggtttggaagaatcggttgctcgcgctcgctatggtcaccgtcgtgtggctgacactggagagtgtcgtgactcgggttgctgtgttggttgtcctcctctgtttggccccagtgtacgcgtccaggtgtactcatttggaaaacagagattttgtcaccggcacccaggggacgactcgggtaaccctggtgcttgaactgggtggttgcgttactattaccgctgagggcaaaccctctatggatgtgtggctggatgcaatctatcaggagaatcccgcacaaaccagggaatattgccttcacgcaaagctgtccgatacaaaggtcgcggctaggtgcccaacaatgggaccggccaccctggcggaggaacatcagggaggtacagtgtgcaaacgggaccagagtgatagaggctggggtaatcactgcggcctgttcggcaaaggaagtattgtcgcttgcgtcaaggcagcctgtgaggccaaaaagaaggctactgggcacgtctatgacgccaacaagatcgtttatacagtgaaagtggaaccacacacaggggattacgtggcggccaacgagactcattccggtcgcaaaacggccagcttcaccgtgtcatccgaaaagaccatcctcactatgggggagtatggcgacgtttctctgctctgccgggtggctagcggagtcgacctggcccagacagtcatcctggaactggataaaacagttgagcatctgcctaccgcttggcaggtgcacagggattggtttaacgaccttgccctgccatggaaacatgaaggagcgagaaactggaataatgcagagcgactcgtagaattcggtgcccctcatgccgtgaagatggacgtctacaatctgggtgatcagaccggcgttctccttaaagctctcgctggcgtaccagttgcccacatcgaaggaacgaagtaccacctgaagtcaggccatgtaacttgcgaggtgggcctggagaagttgaaaatgaaaggtcttacgtacacaatgtgtgacaagaccaagttcacatggaagagggcccccacagatagcggccacgatactgtggtgatggaggtgaccttttctggaacaaaaccctgcagaatacccgtgcgggctgtagctcacggatctcccgatgtcaatgttgctatgctgattacacctaaccctaccatcgagaataacggtggtggttttattgagatgcagcttccgccaggcgataacatcatctacgtgggcgaactctcttaccagtggtttcagaaagggagttcaattgggcgggtcttccaaaaaacgaagaagggaatcgaacgattgacggttatcggcgagcacgcatgggattttggttccgcagggggattcctgtcttctattggtaaggcactgcataccgtgctggggggcgcattcaattctattttcgggggcgtggggttcctgcctaaactcctgctgggagtagccctggcctggttgggactgaatatgcggaatccgacgatgtccatgtcattcctcttggccggcgtgcttgtactggccatgacactgggcgttggcgccgacactgggtgtgccatagacatcagccggcaa

SEQ ID NO: 57 - DNA, 2280 bp, Artificial Sequence
  Synthetic Construct
tcatcaagcggacacactcgactgtttgaatcatcacaaacactcctaattgttgttaattgtgtcacgctcgacaaagaatcgtgcttctagagctacagattctttggtcctcccgggccgttctcggcccgacagataaacgattttgcgccttacggggcgcacaacaggaactaacctgaattcgcctcgaggttcgttttctttgcgccccctttctgtccgtatcgacactactatccgtacgaccgaacacagcctcgtcgatggcacgcttttcttgcgctgccttcgtggcactattcccgactcccattcctacgccgatgcgtccactctcatctcttaccgtgaacgcattatgagcgctgactataccctaggaccacactgctatcggagtcaatacttacgcattgttatctggtcccgcttcttggacacctgcaactgacaaagacatctttacacctaccgcaaatagacctcatgccggcgacaccttttgtcctcccgagtgcttgagcttctagacacgactaaggttcagtgcgcgttcctctcaactggccatctccggtgttcaccgaacttcccctgagtaactcctgggtggactgatcccacctcccaacccaaaccttcttagccaacgagcgcgagcgataccagtggcagcacaccgactgtgacctctcacagcactgagcccaacgacacaaccaacaggaggagacaaaccggggtcacatgcgcaggtccacatgagtaaaccttttgtctctaaaacagtggccgtgggtcccctgctgagcccattgggaccacgaacttgacccaccaacgcaatgataatggcgactcccgtttgggagatacctacacaccgacctacgttagatagtcctcttagggcgtgtttggtcccttataacggaagtgcgtttcgacaggctatgtttccagcgccgatccacgggttgttaccctggccggtgggaccgcctccttgtagtccctccatgtcacacgtttgccctggtctcactatctccgaccccattagtgacgccggacaagccgtttccttcataacagcgaacgcagttccgtcggacactccggtttttcttccgatgacccgtgcagatactgcggttgttctagcaaatatgtcactttcaccttggtgtgtgtcccctaatgcaccgccggttgctctgagtaaggccagcgttttgccggtcgaagtggcacagtaggcttttctggtaggagtgataccccctcataccgctgcaaagagacgagacggcccaccgatcgcctcagctggaccgggtctgtcagtaggaccttgacctattttgtcaactcgtagacggatggcgaaccgtccacgtgtccctaaccaaattgctggaacgggacggtacctttgtacttcctcgctctttgaccttattacgtctcgctgagcatcttaagccacggggagtacggcacttctacctgcagatgttagacccactagtctggccgcaagaggaatttcgagagcgaccgcatggtcaacgggtgtagcttccttgcttcatggtggacttcagtccggtacattgaacgctccacccggacctcttcaacttttactttccagaatgcatgtgttacacactgttctggttcaagtgtaccttctcccgggggtgtctatcgccggtgctatgacaccactacctccactggaaaagaccttgttttgggacgtcttatgggcacgcccgacatcgagtgcctagagggctacagttacaacgatacgactaatgtggattgggatggtagctcttattgccaccaccaaaataactctacgtcgaaggcggtccgctattgtagtagatgcacccgcttgagagaatggtcaccaaagtctttccctcaagttaacccgcccagaaggttttttgcttcttcccttagcttgctaactgccaatagccgctcgtgcgtaccctaaaaccaaggcgtccccctaaggacagaagataaccattccgtgacgtatggcacgaccccccgcgtaagttaagataaaagcccccgcaccccaaggacggatttgaggacgaccctcatcgggaccggaccaaccctgacttatacgccttaggctgctacaggtacagtaaggagaaccggccgcacgaacatgaccggtactgtgacccgcaaccgcggctgtgacccacacggtatctgtagtcggccgtt

SEQ ID NO: 58 - protein, 635 aa, Artificial Seqeunce
  Synthetic Construct
MSKKPGGPGKSRAVYLLKRGMPRVLSLIGLKQKKRGGKTGIAVIVPQALLFVPLLVFPLCFGKFPIYTIPDKLGPWSPIDIHHLSCPNNLVVEDEGCTNLSGFSYMELKVGYISAIKMNGFTCTGVVTEAETYTNFVGYVTTTFKRKHFRPTPDACRAAYNWKMAGDPRYEESLHNPYPDYHWLRTVKTTKESLVIISPSVADLDPYDRSLHSRVFPGGNCSGVAVSSTYCSTNHDYTIWMPENPRLGMSCDIFTNSRGKRASKGSETCGFVDERGLYKSLKGACKLKLCGVLGLRLMDGTWVAMQTSNETKWCPPGQLVNLHDFRSDEIEHLVVEELVKKREECLDALESIMTTKSVSFRRLSHLRKLVPGFGKAYTIFNKTLMEADAHYKSVRTWNEIIPSKGCLRVGGRCHPHVNGVFFNGIILGPDGNVLIPEMQSSLLQQHMELLVSSVIPLMHPLADPSTVFKNGDEAEDFVEVHLPDVHERISGVDLGLPNWGKYVLLSAGALTALMLIIFLMTCWRRVNRSEPTQHNLRGTGREVSVTPQSGKIISSWESYKSGGETGLNFDLLKLAGDVESNPGPARDRSIALTFLAVGGVLLFLSVNVHADTGCAIDISRQELRCGSGVFIHNDV

SEQ ID NO: 59 - DNA, 2000 bp, Artificial Sequence
  Synthetic Construct
agtagttcgcctgtgtgagctgacaaacttagtagtgtttgtgaggattaacaacaattaacacagtgcgagctgtttcttagcacgaagatctcgatgtctaagaaaccaggagggcccggcaagagccgggctgtctatttgctaaaacgcggaatgccccgcgtgttgtccttgattggacttaagcaaaagaagcgagggggcaagactggtatagctgtgatcgttcctcaggctcttttgtttgtacccttgctggtatttcccctttgctttggtaaatttcctatctataccatccctgataagctcgggccttggagtcccattgatattcaccatttgagctgcccaaacaacctcgtcgttgaggatgaagggtgcactaatctttctggattttcctacatggagttgaaagtgggctatatttcagccattaagatgaacggctttacttgtacaggagtcgtgaccgaagccgagacatatacaaatttcgtgggatacgtcaccaccaccttcaagagaaaacacttccgcccaacgcctgacgcttgtcgggccgcttacaactggaagatggcaggagatcctcgatatgaagaatctctgcacaacccgtatcctgattaccattggctgcggacagtcaagactaccaaggagagtctggtcattatatcaccaagcgtggccgatcttgatccttatgatagatccctgcacagtagggtttttcctggcgggaattgtagcggtgttgcagtatcaagtacctactgctccactaaccacgactacactatatggatgcctgagaaccctcgactcggtatgagttgcgacatttttacgaactcacggggcaagcgggcatctaaggggtctgaaacatgcgggtttgttgatgagcgggggttgtataaatctcttaaaggcgcctgtaagctgaaactctgtggcgtactggggctgcgcctgatggacggcacatgggtggctatgcagacaagcaatgaaacaaagtggtgtccccctggtcagctggttaatctgcacgactttaggtctgacgaaatcgagcaccttgtggtggaggaactggtgaagaaacgcgaagagtgcctggacgcacttgagagtattatgaccaccaaatccgtttccttcagaagactgagccacctgcgaaagctggtgccagggttcgggaaggcttatactattttcaacaagactcttatggaggcggatgcccattataagtcagttaggacttggaatgagataattccctccaaaggatgtctgagagtcggtgggagatgccacccccatgtcaatggggtgttctttaacggaatcatcctgggacctgacgggaacgtgctgattcccgagatgcaatcttcccttctgcagcaacacatggaactcctggtgtcttcagtgatacccctgatgcacccactggccgaccccagcactgtgttcaaaaatggcgatgaggccgaagactttgtggaagttcacctgcccgatgtacacgaaaggatatctggagtagacctgggccttcctaattggggtaagtacgtgctcctgagtgcgggtgccttgaccgctttgatgctgatcatttttctgatgacctgctggcggagggtgaatcgctccgagccgacacagcacaatctcagagggacaggccgggaagtaagtgtgactccgcaatctggcaagattattagtagttgggagagttacaagtctggaggagagactgggttgaattttgatctgctcaaacttgcaggcgatgtagaatcaaatcctggacccgcccgggacaggtccatagctctcacgtttctcgcagttggaggagttctgctcttcctctccgtgaacgtgcacgctgacactgggtgtgccatagacatcagccggcaagagctgagatgtggaagtggagtgttcatacacaatgatgt

SEQ ID NO: 60 - DNA, 2000 bp, Artificial Sequence
  Synthetic Construct
tcatcaagcggacacactcgactgtttgaatcatcacaaacactcctaattgttgttaattgtgtcacgctcgacaaagaatcgtgcttctagagctacagattctttggtcctcccgggccgttctcggcccgacagataaacgattttgcgccttacggggcgcacaacaggaactaacctgaattcgttttcttcgctcccccgttctgaccatatcgacactagcaaggagtccgagaaaacaaacatgggaacgaccataaaggggaaacgaaaccatttaaaggatagatatggtagggactattcgagcccggaacctcagggtaactataagtggtaaactcgacgggtttgttggagcagcaactcctacttcccacgtgattagaaagacctaaaaggatgtacctcaactttcacccgatataaagtcggtaattctacttgccgaaatgaacatgtcctcagcactggcttcggctctgtatatgtttaaagcaccctatgcagtggtggtggaagttctcttttgtgaaggcgggttgcggactgcgaacagcccggcgaatgttgaccttctaccgtcctctaggagctatacttcttagagacgtgttgggcataggactaatggtaaccgacgcctgtcagttctgatggttcctctcagaccagtaatatagtggttcgcaccggctagaactaggaatactatctagggacgtgtcatcccaaaaaggaccgcccttaacatcgccacaacgtcatagttcatggatgacgaggtgattggtgctgatgtgatatacctacggactcttgggagctgagccatactcaacgctgtaaaaatgcttgagtgccccgttcgcccgtagattccccagactttgtacgcccaaacaactactcgcccccaacatatttagagaatttccgcggacattcgactttgagacaccgcatgaccccgacgcggactacctgccgtgtacccaccgatacgtctgttcgttactttgtttcaccacagggggaccagtcgaccaattagacgtgctgaaatccagactgctttagctcgtggaacaccacctccttgaccacttctttgcgcttctcacggacctgcgtgaactctcataatactggtggtttaggcaaaggaagtcttctgactcggtggacgctttcgaccacggtcccaagcccttccgaatatgataaaagttgttctgagaatacctccgcctacgggtaatattcagtcaatcctgaaccttactctattaagggaggtttcctacagactctcagccaccctctacggtgggggtacagttaccccacaagaaattgccttagtaggaccctggactgcccttgcacgactaagggctctacgttagaagggaagacgtcgttgtgtaccttgaggaccacagaagtcactatggggactacgtgggtgaccggctggggtcgtgacacaagtttttaccgctactccggcttctgaaacaccttcaagtggacgggctacatgtgctttcctatagacctcatctggacccggaaggattaaccccattcatgcacgaggactcacgcccacggaactggcgaaactacgactagtaaaaagactactggacgaccgcctcccacttagcgaggctcggctgtgtcgtgttagagtctccctgtccggcccttcattcacactgaggcgttagaccgttctaataatcatcaaccctctcaatgttcagacctcctctctgacccaacttaaaactagacgagtttgaacgtccgctacatcttagtttaggacctgggcgggccctgtccaggtatcgagagtgcaaagagcgtcaacctcctcaagacgagaaggagaggcacttgcacgtgcgactgtgacccacacggtatctgtagtcggccgttctcgactctacaccttcacctcacaagtatgtgttactaca

SEQ ID NO: 61 - protein, 1303 aa, Artificial Sequence
  Synthetic Construct
MSKKPGGPGKSRAVNMLKRGMPRVLSLIGLKQKKRGGKTGIAVIVPQALLFVPLLVFPLCFGKFPIYTIPDKLGPWSPIDIHHLSCPNNLVVEDEGCTNLSGFSYMELKVGYISAIKMNGFTCTGVVTEAETYTNFVGYVTTTFKRKHFRPTPDACRAAYNWKMAGDPRYEESLHNPYPDYHWLRTVKTTKESLVIISPSVADLDPYDRSLHSRVFPGGNCSGVAVSSTYCSTNHDYTIWMPENPRLGMSCDIFTNSRGKRASKGSETCGFVDERGLYKSLKGACKLKLCGVLGLRLMDGTWVAMQTSNETKWCPPGQLVNLHDFRSDEIEHLVVEELVKKREECLDALESIMTTKSVSFRRLSHLRKLVPGFGKAYTIFNKTLMEADAHYKSVRTWNEIIPSKGCLRVGGRCHPHVNGVFFNGIILGPDGNVLIPEMQSSLLQQHMELLVSSVIPLMHPLADPSTVFKNGDEAEDFVEVHLPDVHERISGVDLGLPNWGKYVLLSAGALTALMLIIFLMTCWRRVNRSEPTQHNLRGTGREVSVTPQSGKIISSWESYKSGGETGLNFDLLKLAGDVESNPGPGGKTGIAVMIGLIACVGAVTLSNFQGKVMMTVNATDVTDVITIPTAAGKNLCIVRAMDVGYMCDDTITYECPVLSAGNDPEDIDCWCTKSAVYVRYGRCTKTRHSRRSRRSLTVQTHGESTLANKKGAWMDSTKATRYLVKTESWILRNPGYALVAAVIGWMLGSNTMQRVVFVVLLLLVAPAYSFNCLGMSNRDFLEGVSGATWVDLVLEGDSCVTIMSKDKPTIDVKMMNMEAANLAEVRSYCYLATVSDLSTKAACPAMGEAHNDKRADPAFVCRQGVVDRGWGNGCGLFGKGSIDTCAKFACSTKAIGRTILKENIKYEVAIFVHGPTTVESHGNYSTQVGATQAGRFSITPAAPSYTLKLGEYGEVTVDCEPRSGIDTNAYYVMTVGTKTFLVHREWFMDLNLPWSSAGSTVWRNRETLMEFEEPHATKQSVIALGSQEGALHQALAGAIPVEFSSNTVKLTSGHLKCRVKMEKLQLKGTTYGVCSKAFKFLGTPADTGHGTVVLELQYTGTDGPCKVPISSVASLNDLTPVGRLVTVNPFVSVATANAKVLIELEPPFGDSYIVVGRGEQQINHHWHKSGSSIGKAFTTTLKGAQRLAALGDTAWDFGSVGGVFTSVGKAVHQVFGGAFRSLFGGMSWITQGLLGALLLWMGINARDRSIALTFLAVGGVLLFLSVNVEHADTGCAIDISRQELRCGSGVFIHNDVEAWMDRY

SEQ ID NO: 62 - DNA, 4000 bp, Artificial Sequence
  Synthetic Construct
agtagttcgcctgtgtgagctgacaaacttagtagtgtttgtgaggattaacaacaattaacacagtgcgagctgtttcttagcacgaagatctcgatgtctaagaaaccaggagggcccggcaagagccgggctgtcaatatgctaaaacgcggaatgccccgcgtgttgtccttgattggacttaagcaaaagaagcgagggggcaagactggtatagctgtgatcgttcctcaggctcttttgtttgtacccttgctggtatttcccctttgctttggtaaatttcctatctataccatccctgataagctcgggccttggagtcccattgatattcaccatttgagctgcccaaacaacctcgtcgttgaggatgaagggtgcactaatctttctggattttcctacatggagttgaaagtgggctatatttcagccattaagatgaacggctttacttgtacaggagtcgtgaccgaagccgagacatatacaaatttcgtgggatacgtcaccaccaccttcaagagaaaacacttccgcccaacgcctgacgcttgtcgggccgcttacaactggaagatggcaggagatcctcgatatgaagaatctctgcacaacccgtatcctgattaccattggctgcggacagtcaagactaccaaggagagtctggtcattatatcaccaagcgtggccgatcttgatccttatgatagatccctgcacagtagggtttttcctggcgggaattgtagcggtgttgcagtatcaagtacctactgctccactaaccacgactacactatatggatgcctgagaaccctcgactcggtatgagttgcgacatttttacgaactcacggggcaagcgggcatctaaggggtctgaaacatgcgggtttgttgatgagcgggggttgtataaatctcttaaaggcgcctgtaagctgaaactctgtggcgtactggggctgcgcctgatggacggcacatgggtggctatgcagacaagcaatgaaacaaagtggtgtccccctggtcagctggttaatctgcacgactttaggtctgacgaaatcgagcaccttgtggtggaggaactggtgaagaaacgcgaagagtgcctggacgcacttgagagtattatgaccaccaaatccgtttccttcagaagactgagccacctgcgaaagctggtgccagggttcgggaaggcttatactattttcaacaagactcttatggaggcggatgcccattataagtcagttaggacttggaatgagataattccctccaaaggatgtctgagagtcggtgggagatgccacccccatgtcaatggggtgttctttaacggaatcatcctgggacctgacgggaacgtgctgattcccgagatgcaatcttcccttctgcagcaacacatggaactcctggtgtcttcagtgatacccctgatgcacccactggccgaccccagcactgtgttcaaaaatggcgatgaggccgaagactttgtggaagttcacctgcccgatgtacacgaaaggatatctggagtagacctgggccttcctaattggggtaagtacgtgctcctgagtgcgggtgccttgaccgctttgatgctgatcatttttctgatgacctgctggcggagggtgaatcgctccgagccgacacagcacaatctcagagggacaggccgggaagtaagtgtgactccgcaatctggcaagattattagtagttgggagagttacaagtctggaggagagactgggttgaattttgatctgctcaaacttgcaggcgatgtagaatcaaatcctggacccggaggaaagaccggtattgcagtcatgattggcctgatcgcctgcgtaggagcagttaccctctctaacttccaagggaaggtgatgatgacggtaaatgctactgacgtcacagatgtcatcacgattccaacagctgctggaaagaacctatgcattgtcagagcaatggatgtgggatacatgtgcgatgatactatcacttatgaatgcccagtgctgtcggctggtaatgatccagaagacatcgactgttggtgcacaaagtcagcagtctacgtcaggtatggaagatgcaccaagacacgccactcaagacgcagtcggaggtcactgacagtgcagacacacggagaaagcactctagcgaacaagaagggggcttggatggacagcaccaaggccacaaggtatttggtaaaaacagaatcatggatcttgaggaaccctggatatgccctggtggcagccgtcattggttggatgcttgggagcaacaccatgcagagagttgtgtttgtcgtgctattgcttttggtggccccagcttacagctttaactgccttggaatgagcaacagagacttcttggaaggagtgtctggagcaacatgggtggatttggttctcgaaggcgacagctgcgtgactatcatgtctaaggacaagcctaccatcgatgtgaagatgatgaatatggaggcggccaacctggcagaggtccgcagttattgctatttggctaccgtcagcgatctctccaccaaagctgcgtgcccggccatgggagaagctcacaatgacaaacgtgctgacccagcttttgtgtgcagacaaggagtggtggacaggggctggggcaacggctgcggactatttggcaaaggaagcattgacacatgcgccaaatttgcctgctctaccaaggcaataggaagaaccattttgaaagagaatatcaagtacgaagtggccatttttgtccatggaccaactactgtggagtcgcacggaaactactccacacaggttggagccactcaggcagggagattcagcatcactcctgcggcgccttcatacacactaaagcttggagaatatggagaggtgacagtggactgtgaaccacggtcagggattgacaccaatgcatactacgtgatgactgttggaacaaagacgttcttggtccatcgtgagtggttcatggacctcaacctcccttggagcagtgctggaagtactgtgtggaggaacagagagacgttaatggagtttgaggaaccacacgccacgaagcagtctgtgatagcattgggctcacaagagggagctctgcatcaagctttggctggagccattcctgtggaattttcaagcaacactgtcaagttgacgtcgggtcatttgaagtgtagagtgaagatggaaaaattgcagttgaagggaacaacctatggcgtctgttcaaaggctttcaagtttcttgggactcccgcagacacaggtcacggcactgtggtgttggaattgcagtacactggcacggatggaccttgcaaagttcctatctcgtcagtggcttcattgaacgacctaacgccagtgggcagattggtcactgtcaacccttttgtttcagtggccacggccaacgctaaggtcctgattgaattggaaccaccctttggagactcatacatagtggtgggcagaggagaacaacagatcaatcaccactggcacaagtctggaagcagcattggcaaagcctttacaaccaccctcaaaggagcgcagagactagccgctctaggagacacagcttgggactttggatcagttggaggggtgttcacctcagttgggaaggctgtccatcaagtgttcggaggagcattccgctcactgttcggaggcatgtcctggataacgcaaggattgctgggggctctcctgttgtggatgggcatcaatgctcgtgacaggtccatagctctcacgtttctcgcagttggaggagttctgctcttcctctccgtgaacgtgcacgctgacactgggtgtgccatagacatcagccggcaagagctgagatgtggaagtggagtgttcatacacaatgatgtggaggcttggatggaccggt

SEQ ID NO: 63 - DNA, 4000 bp, Artificial Sequence
  Synthetic Construct
tcatcaagcggacacactcgactgtttgaatcatcacaaacactcctaattgttgttaattgtgtcacgctcgacaaagaatcgtgcttctagagctacagattctttggtcctcccgggccgttctcggcccgacagttatacgattttgcgccttacggggcgcacaacaggaactaacctgaattcgttttcttcgctcccccgttctgaccatatcgacactagcaaggagtccgagaaaacaaacatgggaacgaccataaaggggaaacgaaaccatttaaaggatagatatggtagggactattcgagcccggaacctcagggtaactataagtggtaaactcgacgggtttgttggagcagcaactcctacttcccacgtgattagaaagacctaaaaggatgtacctcaactttcacccgatataaagtcggtaattctacttgccgaaatgaacatgtcctcagcactggcttcggctctgtatatgtttaaagcaccctatgcagtggtggtggaagttctcttttgtgaaggcgggttgcggactgcgaacagcccggcgaatgttgaccttctaccgtcctctaggagctatacttcttagagacgtgttgggcataggactaatggtaaccgacgcctgtcagttctgatggttcctctcagaccagtaatatagtggttcgcaccggctagaactaggaatactatctagggacgtgtcatcccaaaaaggaccgcccttaacatcgccacaacgtcatagttcatggatgacgaggtgattggtgctgatgtgatatacctacggactcttgggagctgagccatactcaacgctgtaaaaatgcttgagtgccccgttcgcccgtagattccccagactttgtacgcccaaacaactactcgcccccaacatatttagagaatttccgcggacattcgactttgagacaccgcatgaccccgacgcggactacctgccgtgtacccaccgatacgtctgttcgttactttgtttcaccacagggggaccagtcgaccaattagacgtgctgaaatccagactgctttagctcgtggaacaccacctccttgaccacttctttgcgcttctcacggacctgcgtgaactctcataatactggtggtttaggcaaaggaagtcttctgactcggtggacgctttcgaccacggtcccaagcccttccgaatatgataaaagttgttctgagaatacctccgcctacgggtaatattcagtcaatcctgaaccttactctattaagggaggtttcctacagactctcagccaccctctacggtgggggtacagttaccccacaagaaattgccttagtaggaccctggactgcccttgcacgactaagggctctacgttagaagggaagacgtcgttgtgtaccttgaggaccacagaagtcactatggggactacgtgggtgaccggctggggtcgtgacacaagtttttaccgctactccggcttctgaaacaccttcaagtggacgggctacatgtgctttcctatagacctcatctggacccggaaggattaaccccattcatgcacgaggactcacgcccacggaactggcgaaactacgactagtaaaaagactactggacgaccgcctcccacttagcgaggctcggctgtgtcgtgttagagtctccctgtccggcccttcattcacactgaggcgttagaccgttctaataatcatcaaccctctcaatgttcagacctcctctctgacccaacttaaaactagacgagtttgaacgtccgctacatcttagtttaggacctgggcctcctttctggccataacgtcagtactaaccggactagcggacgcatcctcgtcaatgggagagattgaaggttcccttccactactactgccatttacgatgactgcagtgtctacagtagtgctaaggttgtcgacgacctttcttggatacgtaacagtctcgttacctacaccctatgtacacgctactatgatagtgaatacttacgggtcacgacagccgaccattactaggtcttctgtagctgacaaccacgtgtttcagtcgtcagatgcagtccataccttctacgtggttctgtgcggtgagttctgcgtcagcctccagtgactgtcacgtctgtgtgcctctttcgtgagatcgcttgttcttcccccgaacctacctgtcgtggttccggtgttccataaaccatttttgtcttagtacctagaactccttgggacctatacgggaccaccgtcggcagtaaccaacctacgaaccctcgttgtggtacgtctctcaacacaaacagcacgataacgaaaaccaccggggtcgaatgtcgaaattgacggaaccttactcgttgtctctgaagaaccttcctcacagacctcgttgtacccacctaaaccaagagcttccgctgtcgacgcactgatagtacagattcctgttcggatggtagctacacttctactacttatacctccgccggttggaccgtctccaggcgtcaataacgataaaccgatggcagtcgctagagaggtggtttcgacgcacgggccggtaccctcttcgagtgttactgtttgcacgactgggtcgaaaacacacgtctgttcctcaccacctgtccccgaccccgttgccgacgcctgataaaccgtttccttcgtaactgtgtacgcggtttaaacggacgagatggttccgttatccttcttggtaaaactttctcttatagttcatgcttcaccggtaaaaacaggtacctggttgatgacacctcagcgtgcctttgatgaggtgtgtccaacctcggtgagtccgtccctctaagtcgtagtgaggacgccgcggaagtatgtgtgatttcgaacctcttatacctctccactgtcacctgacacttggtgccagtccctaactgtggttacgtatgatgcactactgacaaccttgtttctgcaagaaccaggtagcactcaccaagtacctggagttggagggaacctcgtcacgaccttcatgacacacctccttgtctctctgcaattacctcaaactccttggtgtgcggtgcttcgtcagacactatcgtaacccgagtgttctccctcgagacgtagttcgaaaccgacctcggtaaggacaccttaaaagttcgttgtgacagttcaactgcagcccagtaaacttcacatctcacttctacctttttaacgtcaacttcccttgttggataccgcagacaagtttccgaaagttcaaagaaccctgagggcgtctgtgtccagtgccgtgacaccacaaccttaacgtcatgtgaccgtgcctacctggaacgtttcaaggatagagcagtcaccgaagtaacttgctggattgcggtcacccgtctaaccagtgacagttgggaaaacaaagtcaccggtgccggttgcgattccaggactaacttaaccttggtgggaaacctctgagtatgtatcaccacccgtctcctcttgttgtctagttagtggtgaccgtgttcagaccttcgtcgtaaccgtttcggaaatgttggtgggagtttcctcgcgtctctgatcggcgagatcctctgtgtcgaaccctgaaacctagtcaacctccccacaagtggagtcaacccttccgacaggtagttcacaagcctcctcgtaaggcgagtgacaagcctccgtacaggacctattgcgttcctaacgacccccgagaggacaacacctacccgtagttacgagcactgtccaggtatcgagagtgcaaagagcgtcaacctcctcaagacgagaaggagaggcacttgcacgtgcgactgtgacccacacggtatctgtagtcggccgttctcgactctacaccttcacctcacaagtatgtgttactacacctccgaacctacctggcca

SEQ ID NO: 64 - protein, 702 aa, Artificial Sequence
  Synthetic Construct
MSKKPGGPGKSRAVYLLKRGMPRVLSLIGLKRAMLSLIDGKGPIRFVLALLAFFRFTAIAPTRAVLDRWRGVNKQTAMKHLLSFKKELGTLTSAINRRSSKQKKRGGKTGIAVIVPQALLFVPLLVFPLCFGKFPIYTIPDKLGPWSPIDIHHLSCPNNLVVEDEGCTNLSGFSYMELKVGYISAIKMNGFTCTGVVTEAETYTNFVGYVTTTFKRKHFRPTPDACRAAYNWKMAGDPRYEESLHNPYPDYHWLRTVKTTKESLVIISPSVADLDPYDRSLHSRVFPGGNCSGVAVSSTYCSTNHDYTIWMPENPRLGMSCDIFTNSRGKRASKGSETCGFVDERGLYKSLKGACKLKLCGVLGLRLMDGTWVAMQTSNETKWCPPGQLVNLHDFRSDEIEHLVVEELVKKREECLDALESIMTTKSVSFRRLSHLRKLVPGFGKAYTIFNKTLMEADAHYKSVRTWNEIIPSKGCLRVGGRCHPHVNGVFFNGIILGPDGNVLIPEMQSSLLQQHMELLVSSVIPLMHPLADPSTVFKNGDEAEDFVEVHLPDVHERISGVDLGLPNWGKYVLLSAGALTALMLIIFLMTCWRRVNRSEPTQHNLRGTGREVSVTPQSGKIISSWESYKSGGETGLNFDLLKLAGDVESNPGPARDRSIALTFLAVGGVLLFLSVNVHADTGCAIDISRQELRCGSGVFIH

SEQ ID NO: 65 - DNA, 2200 bp, Artificial Sequence
  Synthetic Construct
agtagttcgcctgtgtgagctgacaaacttagtagtgtttgtgaggattaacaacaattaacacagtgcgagctgtttcttagcacgaagatctcgatgtctaagaaaccaggagggcccggcaagagccgggctgtctatttgctaaaacgcggaatgccccgcgtgttgtccttgattggacttaagagggctatgttgagcctgatcgacggcaaggggccaatacgatttgtgttggctctcttggcgttcttcaggttcacagcaattgctccgacccgagcagtgctggatcgatggagaggtgtgaacaaacaaacagcgatgaaacaccttctgagtttcaagaaggaactagggaccttgaccagtgctatcaatcggcggagctcaaagcaaaagaagcgagggggcaagactggtatagctgtgatcgttcctcaggctcttttgtttgtacccttgctggtatttcccctttgctttggtaaatttcctatctataccatccctgataagctcgggccttggagtcccattgatattcaccatttgagctgcccaaacaacctcgtcgttgaggatgaagggtgcactaatctttctggattttcctacatggagttgaaagtgggctatatttcagccattaagatgaacggctttacttgtacaggagtcgtgaccgaagccgagacatatacaaatttcgtgggatacgtcaccaccaccttcaagagaaaacacttccgcccaacgcctgacgcttgtcgggccgcttacaactggaagatggcaggagatcctcgatatgaagaatctctgcacaacccgtatcctgattaccattggctgcggacagtcaagactaccaaggagagtctggtcattatatcaccaagcgtggccgatcttgatccttatgatagatccctgcacagtagggtttttcctggcgggaattgtagcggtgttgcagtatcaagtacctactgctccactaaccacgactacactatatggatgcctgagaaccctcgactcggtatgagttgcgacatttttacgaactcacggggcaagcgggcatctaaggggtctgaaacatgcgggtttgttgatgagcgggggttgtataaatctcttaaaggcgcctgtaagctgaaactctgtggcgtactggggctgcgcctgatggacggcacatgggtggctatgcagacaagcaatgaaacaaagtggtgtccccctggtcagctggttaatctgcacgactttaggtctgacgaaatcgagcaccttgtggtggaggaactggtgaagaaacgcgaagagtgcctggacgcacttgagagtattatgaccaccaaatccgtttccttcagaagactgagccacctgcgaaagctggtgccagggttcgggaaggcttatactattttcaacaagactcttatggaggcggatgcccattataagtcagttaggacttggaatgagataattccctccaaaggatgtctgagagtcggtgggagatgccacccccatgtcaatggggtgttctttaacggaatcatcctgggacctgacgggaacgtgctgattcccgagatgcaatcttcccttctgcagcaacacatggaactcctggtgtcttcagtgatacccctgatgcacccactggccgaccccagcactgtgttcaaaaatggcgatgaggccgaagactttgtggaagttcacctgcccgatgtacacgaaaggatatctggagtagacctgggccttcctaattggggtaagtacgtgctcctgagtgcgggtgccttgaccgctttgatgctgatcatttttctgatgacctgctggcggagggtgaatcgctccgagccgacacagcacaatctcagagggacaggccgggaagtaagtgtgactccgcaatctggcaagattattagtagttgggagagttacaagtctggaggagagactgggttgaattttgatctgctcaaacttgcaggcgatgtagaatcaaatcctggacccgcccgggacaggtccatagctctcacgtttctcgcagttggaggagttctgctcttcctctccgtgaacgtgcacgctgacactgggtgtgccatagacatcagccggcaagagctgagatgtggaagtggagtgttcatac

SEQ ID NO: 66 - DNA, 2200 bp, Artificial Sequence
  Synthetic Construct
tcatcaagcggacacactcgactgtttgaatcatcacaaacactcctaattgttgttaattgtgtcacgctcgacaaagaatcgtgcttctagagctacagattctttggtcctcccgggccgttctcggcccgacagataaacgattttgcgccttacggggcgcacaacaggaactaacctgaattctcccgatacaactcggactagctgccgttccccggttatgctaaacacaaccgagagaaccgcaagaagtccaagtgtcgttaacgaggctgggctcgtcacgacctagctacctctccacacttgtttgtttgtcgctactttgtggaagactcaaagttcttccttgatccctggaactggtcacgatagttagccgcctcgagtttcgttttcttcgctcccccgttctgaccatatcgacactagcaaggagtccgagaaaacaaacatgggaacgaccataaaggggaaacgaaaccatttaaaggatagatatggtagggactattcgagcccggaacctcagggtaactataagtggtaaactcgacgggtttgttggagcagcaactcctacttcccacgtgattagaaagacctaaaaggatgtacctcaactttcacccgatataaagtcggtaattctacttgccgaaatgaacatgtcctcagcactggcttcggctctgtatatgtttaaagcaccctatgcagtggtggtggaagttctcttttgtgaaggcgggttgcggactgcgaacagcccggcgaatgttgaccttctaccgtcctctaggagctatacttcttagagacgtgttgggcataggactaatggtaaccgacgcctgtcagttctgatggttcctctcagaccagtaatatagtggttcgcaccggctagaactaggaatactatctagggacgtgtcatcccaaaaaggaccgcccttaacatcgccacaacgtcatagttcatggatgacgaggtgattggtgctgatgtgatatacctacggactcttgggagctgagccatactcaacgctgtaaaaatgcttgagtgccccgttcgcccgtagattccccagactttgtacgcccaaacaactactcgcccccaacatatttagagaatttccgcggacattcgactttgagacaccgcatgaccccgacgcggactacctgccgtgtacccaccgatacgtctgttcgttactttgtttcaccacagggggaccagtcgaccaattagacgtgctgaaatccagactgctttagctcgtggaacaccacctccttgaccacttctttgcgcttctcacggacctgcgtgaactctcataatactggtggtttaggcaaaggaagtcttctgactcggtggacgctttcgaccacggtcccaagcccttccgaatatgataaaagttgttctgagaatacctccgcctacgggtaatattcagtcaatcctgaaccttactctattaagggaggtttcctacagactctcagccaccctctacggtgggggtacagttaccccacaagaaattgccttagtaggaccctggactgcccttgcacgactaagggctctacgttagaagggaagacgtcgttgtgtaccttgaggaccacagaagtcactatggggactacgtgggtgaccggctggggtcgtgacacaagtttttaccgctactccggcttctgaaacaccttcaagtggacgggctacatgtgctttcctatagacctcatctggacccggaaggattaaccccattcatgcacgaggactcacgcccacggaactggcgaaactacgactagtaaaaagactactggacgaccgcctcccacttagcgaggctcggctgtgtcgtgttagagtctccctgtccggcccttcattcacactgaggcgttagaccgttctaataatcatcaaccctctcaatgttcagacctcctctctgacccaacttaaaactagacgagtttgaacgtccgctacatcttagtttaggacctgggcgggccctgtccaggtatcgagagtgcaaagagcgtcaacctcctcaagacgagaaggagaggcacttgcacgtgcgactgtgacccacacggtatctgtagtcggccgttctcgactctacaccttcacctcacaagtatg

SEQ ID NO: 67 - protein, 868 aa, Artificial Sequence
  Synthetic Construct
MSKKPGGPGKSRAVNMLKRGMPRVLSLIGLKRAMLSLIDGKGPIRFVLALLAFFRFTAIAPTRAVLDRWRGVNKQTAMKHLLSFKKELGTLTSAINRRSSKQKKRGGKTGIAVMIGLIASVGAVTLSNFQGKVMMTVNATDVTDVITIPTAAGKNLCIVRAMDVGYMCDDTITYECPVLSAGNDPEDIDCWCTKSAVYVRYGRCTKTRHSRRSRRSLTVQTHGESTLANKKGAWMDSTKATRYLVKTESWILRNPGYALVAAVIGWMLGSNTMQRVVFVVLLLLVAPAYSFNCLGMSNRDFLEGVSGATWVDLVLEGDSCVTIMSKDKPTIDVKMMNMEAANLAEVRSYCYLATVSDLSTKAACPAMGEAHNDKRADPAFVCRQGVVDRGWGNGCGLFGKGSIDTCAKFACSTKAIGRTILKENIKYEVAIFVHGPTTVESHGNYSTQVGATQAGRFSITPAAPSYTLKLGEYGEVTVDCEPRSGIDTNAYYVMTVGTKTFLVHREWFMDLNLPWSSAGSTVWRNRETLMEFEEPHATKQSVIALGSQEGALHQALAGAIPVEFSSNTVKLTSGHLKCRVKMEKLQLKGTTYGVCSKAFKFLGTPADTGHGTVVLELQYTGTDGPCKVPISSVASLNDLTPVGRLVTVNPFVSVATANAKVLIELEPPFGDSYIVVGRGEQQINHHWHKSGSSIGKAFTTTLKGAQRLAALGDTAWDFGSVGGVFTSVGKAVHQVFGGAFRSLFGGMSWITQGLLGALLLWMGINARDRSIALTFLAVGGVLLFLSVNVHADTGIHRGPATRTTTESGKLITDWCCRSCTLPPLRYQTDSGCWYGMEIRPQRHDEKTLVQSQVNAYNADMIDPFQLGL

SEQ ID NO: 68 - DNA, 2700 bp, Artificial Sequence
  Synthetic Construct
agtagttcgcctgtgtgagctgacaaacttagtagtgtttgtgaggattaacaacaattaacacagtgcgagctgtttcttagcacgaagatctcgatgtctaagaaaccaggagggcccggcaagagccgggctgtcaatatgctaaaacgcggaatgccccgcgtgttgtccttgattggacttaagagggctatgttgagcctgatcgacggcaaggggccaatacgatttgtgttggctctcttggcgttcttcaggttcacagcaattgctccgacccgagcagtgctggatcgatggagaggtgtgaacaaacaaacagcgatgaaacaccttctgagtttcaagaaggaactagggaccttgaccagtgctatcaatcggcggagctcaaaacaaaagaaaagaggaggaaagaccggaattgcagtcatgattggcctgatcgccagcgtaggagcagttaccctctctaacttccaagggaaggtgatgatgacggtaaatgctactgacgtcacagatgtcatcacgattccaacagctgctggaaagaacctatgcattgtcagagcaatggatgtgggatacatgtgcgatgatactatcacttatgaatgcccagtgctgtcggctggtaatgatccagaagacatcgactgttggtgcacaaagtcagcagtctacgtcaggtatggaagatgcaccaagacacgccactcaagacgcagtcggaggtcactgacagtgcagacacacggagaaagcactctagcgaacaagaagggggcttggatggacagcaccaaggccacaaggtatttggtaaaaacagaatcatggatcttgaggaaccctggatatgccctggtggcagccgtcattggttggatgcttgggagcaacaccatgcagagagttgtgtttgtcgtgctattgcttttggtggccccagcttacagctttaactgccttggaatgagcaacagagacttcttggaaggagtgtctggagcaacatgggtggatttggttctcgaaggcgacagctgcgtgactatcatgtctaaggacaagcctaccatcgatgtgaagatgatgaatatggaggcggccaacctggcagaggtccgcagttattgctatttggctaccgtcagcgatctctccaccaaagctgcgtgcccggccatgggagaagctcacaatgacaaacgtgctgacccagcttttgtgtgcagacaaggagtggtggacaggggctggggcaacggctgcggactatttggcaaaggaagcattgacacatgcgccaaatttgcctgctctaccaaggcaataggaagaaccattttgaaagagaatatcaagtacgaagtggccatttttgtccatggaccaactactgtggagtcgcacggaaactactccacacaggttggagccactcaggcagggagattcagcatcactcctgcggcgccttcatacacactaaagcttggagaatatggagaggtgacagtggactgtgaaccacggtcagggattgacaccaatgcatactacgtgatgactgttggaacaaagacgttcttggtccatcgtgagtggttcatggacctcaacctcccttggagcagtgctggaagtactgtgtggaggaacagagagacgttaatggagtttgaggaaccacacgccacgaagcagtctgtgatagcattgggctcacaagagggagctctgcatcaagctttggctggagccattcctgtggaattttcaagcaacactgtcaagttgacgtcgggtcatttgaagtgtagagtgaagatggaaaaattgcagttgaagggaacaacctatggcgtctgttcaaaggctttcaagtttcttgggactcccgcagacacaggtcacggcactgtggtgttggaattgcagtacactggcacggatggaccttgcaaagttcctatctcgtcagtggcttcattgaacgacctaacgccagtgggcagattggtcactgtcaacccttttgtttcagtggccacggccaacgctaaggtcctgattgaattggaaccaccctttggagactcatacatagtggtgggcagaggagaacaacagatcaatcaccactggcacaagtctggaagcagcattggcaaagcctttacaaccaccctcaaaggagcgcagagactagccgctctaggagacacagcttgggactttggatcagttggaggggtgttcacctcagttgggaaggctgtccatcaagtgttcggaggagcattccgctcactgttcggaggcatgtcctggataacgcaaggattgctgggggctctcctgttgtggatgggcatcaatgctcgtgacaggtccatagctctcacgtttctcgcagttggaggagttctgctcttcctctccgtgaacgtgcacgctgacactgggatccaccgtggacctgccactcgcaccaccacagagagcggaaagttgataacagattggtgctgcaggagctgcaccttaccaccactgcgctaccaaactgacagcggctgttggtatggtatggagatcagaccacagagacatgatgaaaagaccctcgtgcagtcacaagtgaatgcttataatgctgatatgattgacccttttcagttgggcctt

SEQ ID NO: 69 - DNA, 2700 bp, Artificial Sequence
  Synthetic Construct
tcatcaagcggacacactcgactgtttgaatcatcacaaacactcctaattgttgttaattgtgtcacgctcgacaaagaatcgtgcttctagagctacagattctttggtcctcccgggccgttctcggcccgacagttatacgattttgcgccttacggggcgcacaacaggaactaacctgaattctcccgatacaactcggactagctgccgttccccggttatgctaaacacaaccgagagaaccgcaagaagtccaagtgtcgttaacgaggctgggctcgtcacgacctagctacctctccacacttgtttgtttgtcgctactttgtggaagactcaaagttcttccttgatccctggaactggtcacgatagttagccgcctcgagttttgttttcttttctcctcctttctggccttaacgtcagtactaaccggactagcggtcgcatcctcgtcaatgggagagattgaaggttcccttccactactactgccatttacgatgactgcagtgtctacagtagtgctaaggttgtcgacgacctttcttggatacgtaacagtctcgttacctacaccctatgtacacgctactatgatagtgaatacttacgggtcacgacagccgaccattactaggtcttctgtagctgacaaccacgtgtttcagtcgtcagatgcagtccataccttctacgtggttctgtgcggtgagttctgcgtcagcctccagtgactgtcacgtctgtgtgcctctttcgtgagatcgcttgttcttcccccgaacctacctgtcgtggttccggtgttccataaaccatttttgtcttagtacctagaactccttgggacctatacgggaccaccgtcggcagtaaccaacctacgaaccctcgttgtggtacgtctctcaacacaaacagcacgataacgaaaaccaccggggtcgaatgtcgaaattgacggaaccttactcgttgtctctgaagaaccttcctcacagacctcgttgtacccacctaaaccaagagcttccgctgtcgacgcactgatagtacagattcctgttcggatggtagctacacttctactacttatacctccgccggttggaccgtctccaggcgtcaataacgataaaccgatggcagtcgctagagaggtggtttcgacgcacgggccggtaccctcttcgagtgttactgtttgcacgactgggtcgaaaacacacgtctgttcctcaccacctgtccccgaccccgttgccgacgcctgataaaccgtttccttcgtaactgtgtacgcggtttaaacggacgagatggttccgttatccttcttggtaaaactttctcttatagttcatgcttcaccggtaaaaacaggtacctggttgatgacacctcagcgtgcctttgatgaggtgtgtccaacctcggtgagtccgtccctctaagtcgtagtgaggacgccgcggaagtatgtgtgatttcgaacctcttatacctctccactgtcacctgacacttggtgccagtccctaactgtggttacgtatgatgcactactgacaaccttgtttctgcaagaaccaggtagcactcaccaagtacctggagttggagggaacctcgtcacgaccttcatgacacacctccttgtctctctgcaattacctcaaactccttggtgtgcggtgcttcgtcagacactatcgtaacccgagtgttctccctcgagacgtagttcgaaaccgacctcggtaaggacaccttaaaagttcgttgtgacagttcaactgcagcccagtaaacttcacatctcacttctacctttttaacgtcaacttcccttgttggataccgcagacaagtttccgaaagttcaaagaaccctgagggcgtctgtgtccagtgccgtgacaccacaaccttaacgtcatgtgaccgtgcctacctggaacgtttcaaggatagagcagtcaccgaagtaacttgctggattgcggtcacccgtctaaccagtgacagttgggaaaacaaagtcaccggtgccggttgcgattccaggactaacttaaccttggtgggaaacctctgagtatgtatcaccacccgtctcctcttgttgtctagttagtggtgaccgtgttcagaccttcgtcgtaaccgtttcggaaatgttggtgggagtttcctcgcgtctctgatcggcgagatcctctgtgtcgaaccctgaaacctagtcaacctccccacaagtggagtcaacccttccgacaggtagttcacaagcctcctcgtaaggcgagtgacaagcctccgtacaggacctattgcgttcctaacgacccccgagaggacaacacctacccgtagttacgagcactgtccaggtatcgagagtgcaaagagcgtcaacctcctcaagacgagaaggagaggcacttgcacgtgcgactgtgaccctaggtggcacctggacggtgagcgtggtggtgtctctcgcctttcaactattgtctaaccacgacgtcctcgacgtggaatggtggtgacgcgatggtttgactgtcgccgacaaccataccatacctctagtctggtgtctctgtactacttttctgggagcacgtcagtgttcacttacgaatattacgactatactaactgggaaaagtcaacccggaa

SEQ ID NO: 70 - protein, 734 aa, Artificial Sequence
  Synthetic Construct
MSKKPGGPGKSRAVYLLKRGMPRVLSLIGLKRAMLSLIDGKGPIRFVLALLAFFRFTAIAPTRAVLDRWRGVNKQTAMKHLLSFKKELGTLTSAINRRSSKQKKRGGELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSNNFDLLKLAGDVESNPGPARDRSIALTFLAVGGVLLFLSVNVHADTGCAIDISRQ

SEQ ID NO: 71 - DNA, 2298 bp, Artificial Sequence
  Synthetic Construct
agtagttcgcctgtgtgagctgacaaacttagtagtgtttgtgaggattaacaacaattaacacagtgcgagctgtttcttagcacgaagatctcgatgtctaagaaaccaggagggcccggcaagagccgggctgtctatttgctaaaacgcggaatgccccgcgtgttgtccttgattggacttaagagggctatgttgagcctgatcgacggcaaggggccaatacgatttgtgttggctctcttggcgttcttcaggttcacagcaattgctccgacccgagcagtgctggatcgatggagaggtgtgaacaaacaaacagcgatgaaacaccttctgagtttcaagaaggaactagggaccttgaccagtgctatcaatcggcggagctcaaagcaaaagaagcgagggggcgagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaaggaaaataagtgtaatggaacagatgctaaggtaaaattgataaaacaagaattagataaatataaaaatgctgtaacagaattgcagttgctcatgcaaagcacaccaccaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagttagtgtcttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaaataaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaagggatggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgctggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaacaattttgatctgctcaaacttgcaggcgatgtagaatcaaatcctggacccgcccgggacaggtccatagctctcacgtttctcgcagttggaggagttctgctcttcctctccgtgaacgtgcacgctgacactgggtgtgccatagacatcagccggcaa

SEQ ID NO: 72 - DNA, 2298 bp, Artificial Sequence
  Synthetic Construct
tcatcaagcggacacactcgactgtttgaatcatcacaaacactcctaattgttgttaattgtgtcacgctcgacaaagaatcgtgcttctagagctacagattctttggtcctcccgggccgttctcggcccgacagataaacgattttgcgccttacggggcgcacaacaggaactaacctgaattctcccgatacaactcggactagctgccgttccccggttatgctaaacacaaccgagagaaccgcaagaagtccaagtgtcgttaacgaggctgggctcgtcacgacctagctacctctccacacttgtttgtttgtcgctactttgtggaagactcaaagttcttccttgatccctggaactggtcacgatagttagccgcctcgagtttcgttttcttcgctcccccgctcaacgattaggagtttcgtttacgttaatggtgttaggagtgacgtcagtgtaaaacaaaacgaagaccagttttgtagtgacttcttaaaatagttagttgtacgtcacgtcaatcgtttccgatagaatcacgagactcttgaccaaccatatggtcacaatattgatatcttaattcattatagttccttttattcacattaccttgtctacgattccattttaactattttgttcttaatctatttatatttttacgacattgtcttaacgtcaacgagtacgtttcgtgtggtggttgtttgttagctcggtcttctcttgatggttccaaatacttaatatgtgagttgttacggtttttttggttacattgtaattcgttcttttccttttcttctaaagaaccaaaaaacaatccacaacctagacgttagcggtcaccgcaacgacatagattccaggacgtggatcttccccttcacttgttctagttttcacgagatgataggtgtttgttccgacatcagtcgaatagtttacctcaatcacagaattggtcgtttcacaatctggagtttttgatatatctatttgttaacaatggataacacttgttcgtttcgacgtcgtatagtttatatctttgacactatctcaaggttgttttcttgttgtctgatgatctctaatggtcccttaaatcacaattacgtccacattgatgtggacattcgtgaatgtacaattgattatcacttaataacagtaattagttactatacggatattgtttactagtctttttcaattacaggttgttacaagtttatcaatctgtcgtttcaatgagatagtacaggtattattttctccttcagaatcgtatacatcatgttaatggtgatataccacaatatctatgtgggacaacctttgatgtgtgtaggggagatacatgttggttgtgttttcttcccaggttgtagacaaattgttcttgactgtctcctaccatgacactgttacgtcctagtcatagaaagaagggtgttcgactttgtacatttcaagttagtttagctcataaaacactgtgttacttgtcaaattgtaatggttcactttatttagagacgttacaactgtataagttggggtttatactaacattttaatactgaagtttttgtctacattcgtcgaggcaatagtgtagagatcctcggtaacacagtacgataccgttttgatttacatgtcgtaggttatttttagcaccttagtatttctgtaaaagattgcccacgctaatacatagtttatttccctacctgtgacacagacatccattgtgtaatataatacatttattcgttcttccattttcagagatacattttccacttggttattatttaaagatactgggtaatcataaggggagactacttaaactacgtagttatagagttcagttgctcttctaattggtctcggatcgtaaataagcatttaggctacttaataatgtattacatttacgaccatttaggtggtgtttatagtactattgatgatattaatatcactaatatcattataacaatagtaattaacgacaacctgacgagaatatgacattccggtcttcgtgtggtcagtgtgattcgtttctagttgactcaccatatttattataacgtaaatcattgttaaaactagacgagtttgaacgtccgctacatcttagtttaggacctgggcgggccctgtccaggtatcgagagtgcaaagagcgtcaacctcctcaagacgagaaggagaggcacttgcacgtgcgactgtgacccacacggtatctgtagtcggccgtt

SEQ ID NO: 73 - protein, 667 aa, Artificial Sequence
  Synthetic Construct
MSKKPGGPGKSRAVNMLKRGMPRVLSLIGLKQKKRGGELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSNNFDLLKLAGDVESNPGPARDRSIALTFLAVGGVLLFLSVNVHADTGCAIDISRQELR

SEQ ID NO: 74 - DNA, 2097 bp, Artificial Sequence
  Synthetic Construct
agtagttcgcctgtgtgagctgacaaacttagtagtgtttgtgaggattaacaacaattaacacagtgcgagctgtttcttagcacgaagatctcgatgtctaagaaaccaggagggcccggcaagagccgggctgtcaatatgctaaaacgcggaatgccccgcgtgttgtccttgattggacttaagcaaaagaagcgagggggcgagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaaggaaaataagtgtaatggaacagatgctaaggtaaaattgataaaacaagaattagataaatataaaaatgctgtaacagaattgcagttgctcatgcaaagcacaccaccaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagttagtgtcttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaaataaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaagggatggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgctggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaacaattttgatctgctcaaacttgcaggcgatgtagaatcaaatcctggacccgcccgggacaggtccatagctctcacgtttctcgcagttggaggagttctgctcttcctctccgtgaacgtgcacgctgacactgggtgtgccatagacatcagccggcaagagctgaga

SEQ ID NO: 75 - DNA, 2097 bp, Artificial Sequence
  Synthetic Construct
tcatcaagcggacacactcgactgtttgaatcatcacaaacactcctaattgttgttaattgtgtcacgctcgacaaagaatcgtgcttctagagctacagattctttggtcctcccgggccgttctcggcccgacagttatacgattttgcgccttacggggcgcacaacaggaactaacctgaattcgttttcttcgctcccccgctcaacgattaggagtttcgtttacgttaatggtgttaggagtgacgtcagtgtaaaacaaaacgaagaccagttttgtagtgacttcttaaaatagttagttgtacgtcacgtcaatcgtttccgatagaatcacgagactcttgaccaaccatatggtcacaatattgatatcttaattcattatagttccttttattcacattaccttgtctacgattccattttaactattttgttcttaatctatttatatttttacgacattgtcttaacgtcaacgagtacgtttcgtgtggtggttgtttgttagctcggtcttctcttgatggttccaaatacttaatatgtgagttgttacggtttttttggttacattgtaattcgttcttttccttttcttctaaagaaccaaaaaacaatccacaacctagacgttagcggtcaccgcaacgacatagattccaggacgtggatcttccccttcacttgttctagttttcacgagatgataggtgtttgttccgacatcagtcgaatagtttacctcaatcacagaattggtcgtttcacaatctggagtttttgatatatctatttgttaacaatggataacacttgttcgtttcgacgtcgtatagtttatatctttgacactatctcaaggttgttttcttgttgtctgatgatctctaatggtcccttaaatcacaattacgtccacattgatgtggacattcgtgaatgtacaattgattatcacttaataacagtaattagttactatacggatattgtttactagtctttttcaattacaggttgttacaagtttatcaatctgtcgtttcaatgagatagtacaggtattattttctccttcagaatcgtatacatcatgttaatggtgatataccacaatatctatgtgggacaacctttgatgtgtgtaggggagatacatgttggttgtgttttcttcccaggttgtagacaaattgttcttgactgtctcctaccatgacactgttacgtcctagtcatagaaagaagggtgttcgactttgtacatttcaagttagtttagctcataaaacactgtgttacttgtcaaattgtaatggttcactttatttagagacgttacaactgtataagttggggtttatactaacattttaatactgaagtttttgtctacattcgtcgaggcaatagtgtagagatcctcggtaacacagtacgataccgttttgatttacatgtcgtaggttatttttagcaccttagtatttctgtaaaagattgcccacgctaatacatagtttatttccctacctgtgacacagacatccattgtgtaatataatacatttattcgttcttccattttcagagatacattttccacttggttattatttaaagatactgggtaatcataaggggagactacttaaactacgtagttatagagttcagttgctcttctaattggtctcggatcgtaaataagcatttaggctacttaataatgtattacatttacgaccatttaggtggtgtttatagtactattgatgatattaatatcactaatatcattataacaatagtaattaacgacaacctgacgagaatatgacattccggtcttcgtgtggtcagtgtgattcgtttctagttgactcaccatatttattataacgtaaatcattgttaaaactagacgagtttgaacgtccgctacatcttagtttaggacctgggcgggccctgtccaggtatcgagagtgcaaagagcgtcaacctcctcaagacgagaaggagaggcacttgcacgtgcgactgtgacccacacggtatctgtagtcggccgttctcgactct

SEQ ID NO: 76 - protein, 1327 aa, Artificial Sequence
  Synthetic Construct
MSKKPGGPGKSRAVNMLKRGMPRVLSLIGLKQKKRGGELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSNNFDLLKLAGDVESNPGPGGKTGIAVMIGLIACVGAVTLSNFQGKVMMTVNATDVTDVITIPTAAGKNLCIVRAMDVGYMCDDTITYECPVLSAGNDPEDIDCWCTKSAVYVRYGRCTKTRHSRRSRRSLTVQTHGESTLANKKGAWMDSTKATRYLVKTESWILRNPGYALVAAVIGWMLGSNTMQRVVFVVLLLLVAPAYSFNCLGMSNRDFLEGVSGATWVDLVLEGDSCVTIMSKDKPTIDVKMMNMEAANLAEVRSYCYLATVSDLSTKAACPAMGEAHNDKRADPAFVCRQGVVDRGWGNGCGLFGKGSIDTCAKFACSTKAIGRTILKENIKYEVAIFVHGPTTVESHGNYSTQVGATQAGRFSITPAAPSYTLKLGEYGEVTVDCEPRSGIDTNAYYVMTVGTKTFLVHREWFMDLNLPWSSAGSTVWRNRETLMEFEEPHATKQSVIALGSQEGALHQALAGAIPVEFSSNTVKLTSGHLKCRVKMEKLQLKGTTYGVCSKAFKFLGTPADTGHGTVVLELQYTGTDGPCKVPISSVASLNDLTPVGRLVTVNPFVSVATANAKVLIELEPPFGDSYIVVGRGEQQINHHWHKSGSSIGKAFTTTLKGAQRLAALGDTAWDFGSVGGVFTSVGKAVHQVFGGAFRSLFGGMSWITQGLLGALLLWMGINARDRSIALTFLAVGGVLLFLSVNVHADTGCAIDISRQELR

SEQ ID NO: 77 - DNA, 4100 bp, Artificial Sequence
  Synthetic Construct
gatcctaatacgactcactatagagtagttcgcctgtgtgagctgacaaacttagtagtgtttgtgaggattaacaacaattaacacagtgcgagctgtttcttagcacgaagatctcgatgtctaagaaaccaggagggcccggcaagagccgggctgtcaatatgctaaaacgcggaatgccccgcgtgttgtccttgattggacttaagcaaaagaagcgagggggcgagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaaggaaaataagtgtaatggaacagatgctaaggtaaaattgataaaacaagaattagataaatataaaaatgctgtaacagaattgcagttgctcatgcaaagcacaccaccaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagttagtgtcttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaaataaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaagggatggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgctggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaacaattttgatctgctcaaacttgcaggcgatgtagaatcaaatcctggacccggaggaaagaccggtattgcagtcatgattggcctgatcgcctgcgtaggagcagttaccctctctaacttccaagggaaggtgatgatgacggtaaatgctactgacgtcacagatgtcatcacgattccaacagctgctggaaagaacctatgcattgtcagagcaatggatgtgggatacatgtgcgatgatactatcacttatgaatgcccagtgctgtcggctggtaatgatccagaagacatcgactgttggtgcacaaagtcagcagtctacgtcaggtatggaagatgcaccaagacacgccactcaagacgcagtcggaggtcactgacagtgcagacacacggagaaagcactctagcgaacaagaagggggcttggatggacagcaccaaggccacaaggtatttggtaaaaacagaatcatggatcttgaggaaccctggatatgccctggtggcagccgtcattggttggatgcttgggagcaacaccatgcagagagttgtgtttgtcgtgctattgcttttggtggccccagcttacagctttaactgccttggaatgagcaacagagacttcttggaaggagtgtctggagcaacatgggtggatttggttctcgaaggcgacagctgcgtgactatcatgtctaaggacaagcctaccatcgatgtgaagatgatgaatatggaggcggccaacctggcagaggtccgcagttattgctatttggctaccgtcagcgatctctccaccaaagctgcgtgcccggccatgggagaagctcacaatgacaaacgtgctgacccagcttttgtgtgcagacaaggagtggtggacaggggctggggcaacggctgcggactatttggcaaaggaagcattgacacatgcgccaaatttgcctgctctaccaaggcaataggaagaaccattttgaaagagaatatcaagtacgaagtggccatttttgtccatggaccaactactgtggagtcgcacggaaactactccacacaggttggagccactcaggcagggagattcagcatcactcctgcggcgccttcatacacactaaagcttggagaatatggagaggtgacagtggactgtgaaccacggtcagggattgacaccaatgcatactacgtgatgactgttggaacaaagacgttcttggtccatcgtgagtggttcatggacctcaacctcccttggagcagtgctggaagtactgtgtggaggaacagagagacgttaatggagtttgaggaaccacacgccacgaagcagtctgtgatagcattgggctcacaagagggagctctgcatcaagctttggctggagccattcctgtggaattttcaagcaacactgtcaagttgacgtcgggtcatttgaagtgtagagtgaagatggaaaaattgcagttgaagggaacaacctatggcgtctgttcaaaggctttcaagtttcttgggactcccgcagacacaggtcacggcactgtggtgttggaattgcagtacactggcacggatggaccttgcaaagttcctatctcgtcagtggcttcattgaacgacctaacgccagtgggcagattggtcactgtcaacccttttgtttcagtggccacggccaacgctaaggtcctgattgaattggaaccaccctttggagactcatacatagtggtgggcagaggagaacaacagatcaatcaccactggcacaagtctggaagcagcattggcaaagcctttacaaccaccctcaaaggagcgcagagactagccgctctaggagacacagcttgggactttggatcagttggaggggtgttcacctcagttgggaaggctgtccatcaagtgttcggaggagcattccgctcactgttcggaggcatgtcctggataacgcaaggattgctgggggctctcctgttgtggatgggcatcaatgctcgtgacaggtccatagctctcacgtttctcgcagttggaggagttctgctcttcctctccgtgaacgtgcacgctgacactgggtgtgccatagacatcagccggcaagagctgaga

SEQ ID NO: 78 - DNA, 4100 bp, Artificial Sequence
  Synthetic Construct
ctaggattatgctgagtgatatctcatcaagcggacacactcgactgtttgaatcatcacaaacactcctaattgttgttaattgtgtcacgctcgacaaagaatcgtgcttctagagctacagattctttggtcctcccgggccgttctcggcccgacagttatacgattttgcgccttacggggcgcacaacaggaactaacctgaattcgttttcttcgctcccccgctcaacgattaggagtttcgtttacgttaatggtgttaggagtgacgtcagtgtaaaacaaaacgaagaccagttttgtagtgacttcttaaaatagttagttgtacgtcacgtcaatcgtttccgatagaatcacgagactcttgaccaaccatatggtcacaatattgatatcttaattcattatagttccttttattcacattaccttgtctacgattccattttaactattttgttcttaatctatttatatttttacgacattgtcttaacgtcaacgagtacgtttcgtgtggtggttgtttgttagctcggtcttctcttgatggttccaaatacttaatatgtgagttgttacggtttttttggttacattgtaattcgttcttttccttttcttctaaagaaccaaaaaacaatccacaacctagacgttagcggtcaccgcaacgacatagattccaggacgtggatcttccccttcacttgttctagttttcacgagatgataggtgtttgttccgacatcagtcgaatagtttacctcaatcacagaattggtcgtttcacaatctggagtttttgatatatctatttgttaacaatggataacacttgttcgtttcgacgtcgtatagtttatatctttgacactatctcaaggttgttttcttgttgtctgatgatctctaatggtcccttaaatcacaattacgtccacattgatgtggacattcgtgaatgtacaattgattatcacttaataacagtaattagttactatacggatattgtttactagtctttttcaattacaggttgttacaagtttatcaatctgtcgtttcaatgagatagtacaggtattattttctccttcagaatcgtatacatcatgttaatggtgatataccacaatatctatgtgggacaacctttgatgtgtgtaggggagatacatgttggttgtgttttcttcccaggttgtagacaaattgttcttgactgtctcctaccatgacactgttacgtcctagtcatagaaagaagggtgttcgactttgtacatttcaagttagtttagctcataaaacactgtgttacttgtcaaattgtaatggttcactttatttagagacgttacaactgtataagttggggtttatactaacattttaatactgaagtttttgtctacattcgtcgaggcaatagtgtagagatcctcggtaacacagtacgataccgttttgatttacatgtcgtaggttatttttagcaccttagtatttctgtaaaagattgcccacgctaatacatagtttatttccctacctgtgacacagacatccattgtgtaatataatacatttattcgttcttccattttcagagatacattttccacttggttattatttaaagatactgggtaatcataaggggagactacttaaactacgtagttatagagttcagttgctcttctaattggtctcggatcgtaaataagcatttaggctacttaataatgtattacatttacgaccatttaggtggtgtttatagtactattgatgatattaatatcactaatatcattataacaatagtaattaacgacaacctgacgagaatatgacattccggtcttcgtgtggtcagtgtgattcgtttctagttgactcaccatatttattataacgtaaatcattgttaaaactagacgagtttgaacgtccgctacatcttagtttaggacctgggcctcctttctggccataacgtcagtactaaccggactagcggacgcatcctcgtcaatgggagagattgaaggttcccttccactactactgccatttacgatgactgcagtgtctacagtagtgctaaggttgtcgacgacctttcttggatacgtaacagtctcgttacctacaccctatgtacacgctactatgatagtgaatacttacgggtcacgacagccgaccattactaggtcttctgtagctgacaaccacgtgtttcagtcgtcagatgcagtccataccttctacgtggttctgtgcggtgagttctgcgtcagcctccagtgactgtcacgtctgtgtgcctctttcgtgagatcgcttgttcttcccccgaacctacctgtcgtggttccggtgttccataaaccatttttgtcttagtacctagaactccttgggacctatacgggaccaccgtcggcagtaaccaacctacgaaccctcgttgtggtacgtctctcaacacaaacagcacgataacgaaaaccaccggggtcgaatgtcgaaattgacggaaccttactcgttgtctctgaagaaccttcctcacagacctcgttgtacccacctaaaccaagagcttccgctgtcgacgcactgatagtacagattcctgttcggatggtagctacacttctactacttatacctccgccggttggaccgtctccaggcgtcaataacgataaaccgatggcagtcgctagagaggtggtttcgacgcacgggccggtaccctcttcgagtgttactgtttgcacgactgggtcgaaaacacacgtctgttcctcaccacctgtccccgaccccgttgccgacgcctgataaaccgtttccttcgtaactgtgtacgcggtttaaacggacgagatggttccgttatccttcttggtaaaactttctcttatagttcatgcttcaccggtaaaaacaggtacctggttgatgacacctcagcgtgcctttgatgaggtgtgtccaacctcggtgagtccgtccctctaagtcgtagtgaggacgccgcggaagtatgtgtgatttcgaacctcttatacctctccactgtcacctgacacttggtgccagtccctaactgtggttacgtatgatgcactactgacaaccttgtttctgcaagaaccaggtagcactcaccaagtacctggagttggagggaacctcgtcacgaccttcatgacacacctccttgtctctctgcaattacctcaaactccttggtgtgcggtgcttcgtcagacactatcgtaacccgagtgttctccctcgagacgtagttcgaaaccgacctcggtaaggacaccttaaaagttcgttgtgacagttcaactgcagcccagtaaacttcacatctcacttctacctttttaacgtcaacttcccttgttggataccgcagacaagtttccgaaagttcaaagaaccctgagggcgtctgtgtccagtgccgtgacaccacaaccttaacgtcatgtgaccgtgcctacctggaacgtttcaaggatagagcagtcaccgaagtaacttgctggattgcggtcacccgtctaaccagtgacagttgggaaaacaaagtcaccggtgccggttgcgattccaggactaacttaaccttggtgggaaacctctgagtatgtatcaccacccgtctcctcttgttgtctagttagtggtgaccgtgttcagaccttcgtcgtaaccgtttcggaaatgttggtgggagtttcctcgcgtctctgatcggcgagatcctctgtgtcgaaccctgaaacctagtcaacctccccacaagtggagtcaacccttccgacaggtagttcacaagcctcctcgtaaggcgagtgacaagcctccgtacaggacctattgcgttcctaacgacccccgagaggacaacacctacccgtagttacgagcactgtccaggtatcgagagtgcaaagagcgtcaacctcctcaagacgagaaggagaggcacttgcacgtgcgactgtgacccacacggtatctgtagtcggccgttctcgactct

SEQ ID NO: 79 - protein, 34 aa, Artificial Sequence
  Synthetic Construct
GGKTGIAVIMELPIIKANAITTILIAVTFCFASS

SEQ ID NO: 80 - protein, 16 aa, Autoprotease FMDV 2A
NFDLLKLAGDVESNPG

SEQ ID NO: 81 - protein, 551 aa, Artificial Sequence
  Synthetic Construct
GGKTGIAVIMELPIIKANAITTILIAVTFCFASSQNITEEFYQSTCSAVSKGYLSALRTGWYTSVITIELSNIKENKCNGTDAKVKLIKQELDKYKNAVTELQLLMQSTPAANNRARRELPRFMNYTLNNAKKTNVTLSKKRKRRFLGFLLGVGSAIASGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCNNAGSVSFFPLADTCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMNFDLLKLAGDVESNPG

SEQ ID NO: 82 - protein, 3325 aa, Artificial Sequence
  Synthetic Construct
MSKKPGGPGKSRAVYLLKRGMPRVLSLIGLKRAMLSLIDGKGPIRFVLALLAFFRFTAIAPTRAVLDRWRGVNKQTAMKHLLSFKKELGTLTSAINRRSSKQKKRGGKTGIAVIMELPIIKANAITTILIAVTFCFASSQNITEEFYQSTCSAVSKGYLSALRTGWYTSVITIELSNIKENKCNGTDAKVKLIKQELDKYKNAVTELQLLMQSTPAANNRARRELPRFMNYTLNNAKKTNVTLSKKRKRRFLGFLLGVGSAIASGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCNNAGSVSFFPLADTCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMNFDLLKLAGDVESNPGPARDRSIALTFLAVGGVLLFLSVNVHADTGCAIDISRQELRCGSGVFIHNDVEAWMDRYKYYPETPQGLAKIIQKAHKEGVCGLRSVSRLEHQMWEAVKDELNTLLKENGVDLSVVVEKQGGMYKSAPKRLTATTEKLEIGWKAWGKSILFAPELANNTFVVDGPETKECPTQNRAWNSLEVEDFGFGLTSTRMFLKVRESNTTECDSKIIGTAVKNNLAIHSDLSYWIESRLNDTWKLERAVLGEVKSCTWPETHTLWGDGILESDLIIPVTLAGPRSNHNRRPGYKTQNQGPWDEGRVEIDFDYCPGTTVTLSESCGHRGPATRTTTESGKLITDWCCRSCTLPPLRYQTDSGCWYGMEIRPQRHDEKTLVQSQVNAYNADMIDPFQLGLLVVFLATQEVLRKRWTAKISMPAILIALLVLVFGGITYTDVLRYVILVGAAFAESNSGGDVVHLALMATFKIQPVFMVASFLKARWTNQENILLMLAAVFFQMAYHDARQILLWEIPDVLNSLAIAWMILRAITFTTTSNVVVPLLALLTPGLRCLNLDVYRILLLMVGIGSLIREKRSAAAKKKGASLLCLALASTGLFNPMILAAGLIACDPNRKRGWPATEVMTAVGLMFAIVGGLAELDIDSMAIPMTIAGLMFAAFVISGKSTDMWIERTADISWESDAEITGSSERVDVRLDDDGNFQLMNDPGAPWKIWMLRMVCLAISAYTPWAILPSVVGFWITLQYTKRGGVLWDTPSPKEYKKGDTTTGVYRIMTRGLLGSYQAGAGVMVEGVFHTLWHTTKGAALMSGEGRLDPYWGSVKEDRLCYGGPWKLQHKWNGQDEVQMIVVEPGKNVKNVQTKPGVFKTPEGEIGAVTLDFPTGTSGSPIVDKNGDVIGLYGNGVIMPNGSYISAIVQGERMDEPIPAGFEPEMLRKKQITVLDLHPGAGKTRRILPQIIKEAINRRLRTAVLAPTRVVAAEMAEALRGLPIRYQTSAVPREHNGNEIVDVMCHATLTHRLMSPHRVPNYNLFVMDEAHFTDPASIAARGYISTKVELGEAAAIFMTATPPGTSDPFPESNSPISDLQTEIPDRAWNSGYEWITEYTGKTVWFVPSVKMGNEIALCLQRAGKKVVQLNRKSYETEYPKCKNDDWDFVITTDISEMGANFKASRVIDSRKSVKPTIITEGEGRVILGEPSAVTAASAAQRRGRIGRNPSQVGDEYCYGGHTNEDDSNFAHWTEARIMLDNINMPNGLIAQFYQPEREKVYTMDGEYRLRGEERKNFLELLRTADLPVWLAYKVAAAGVSYHDRRWCFDGPRTNTILEDNNEVEVITKLGERKILRPRWIDARVYSDHQALKAFKDFASGKRSQIGLIEVLGKMPEHFMGKTWEALDTMYVVATAEKGGRAHRMALEELPDALQTIALIALLSVMTMGVFFLLMQRKGIGKIGLGGAVLGVATFFCWMAEVPGTKIAGMLLLSLLLMIVLIPEPEKQRSQTDNQLAVFLICVMTLVSAVAANEMGWLDKTKSDISSLFGQRIEVKENFSMGEFLLDLRPATAWSLYAVTTAVLTPLLKHLITSDYINTSLTSINVQASALFTLARGFPFVDVGVSALLLAAGCWGQVTLTVTVTAATLLFCHYAYMVPGWQAEAMRSAQRRTAAGIMKNAVVDGIVATDVPELERTTPIMQKKIGQIMLILVSLAAVVVNPSVKTVREAGILITAAAVTLWENGASSVWNATTAIGLCHIMRGGWLSCLSITWTLIKNMEKPGLKRGGAKGRTLGEVWKERLNQMTKEEFTRYRKEAIIEVDRSAAKHARKEGNVTGGHPVSRGTAKLRWLVERRFLEPVGKVIDLGCGRGGWCYYMATQKRVQEVRGYTKGGPGHEEPQLVQSYGWNIVTMKSGVDVFYRPSECCDTLLCDIGESSSSAEVEEHRTIRVLEMVEDWLHRGPREFCVKVLCPYMPKVIEKMELLQRRYGGGLVRNPLSRNSTHEMYWVSRASGNVVHSVNMTSQVLLGRMEKRTWKGPQYEEDVNLGSGTRAVGKPLLNSDTSKIKNRIERLRREYSSTWHHDENHPYRTWNYHGSYDVKPTGSASSLVNGVVRLLSKPWDTITNVTTMAMTDTTPFGQQRVFKEKVDTKAPEPPEGVKYVLNETTNWLWAFLAREKRPRMCSREEFIRKVNSNAALGAMFEEQNQWRSAREAVEDPKFWEMVDEEREAHLRGECHTCIYNMMGKREKKPGEFGKAKGSRAIWFMWLGARFLEFEALGFLNEDHWLGRKNSGGGVEGLGLQKLGYILREVGIRPGGKIYADDTAGWDTRITRADLENEAKVLELLDGEHRRLARAIIELTYRHKVVKVMRPAADGRTVMDVISREDQRGSGQVVTYALNTFTNLAVQLVRMMEGEGVIGPDDVEKLTKGKGPKVRTWLFENGEERLSRMAVSGDDCVVKPLDDRFATSLHFLNAMSKVRKDIQEWKPSTGWYDWQQVPFCSNHFTELIMKDGRTLVVPCRGQDELVGRARISPGAGWNVRDTACLAKSYAQMWLLLYFHRRDLRLMANAICSAVPVNWVPTGRTTWSIHAGGEWMTTEDMLEVWNRVWIEENEWMEDKTPVEKWSDVPYSGKREDIWCGSLIGTRARATWAENIQVAINQVRAIIGDEKYVDYMSSLKRYEDTTLVEDTVL

SEQ ID NO: 83 - DNA, 10705 bp, Artificial Sequence
  Synthetic Construct
agtagttcgcctgtgtgagctgacaaacttagtagtgtttgtgaggattaacaacaattaacacagtgcgagctgtttcttagcacgaagatctcgatgtctaagaaaccaggagggcccggcaagagccgggctgtctatttgctaaaacgcggaatgccccgcgtgttgtccttgattggacttaagagggctatgttgagcctgatcgacggcaaggggccaatacgatttgtgttggctctcttggcgttcttcaggttcacagcaattgctccgacccgagcagtgctggatcgatggagaggtgtgaacaaacaaacagcgatgaaacaccttctgagtttcaagaaggaactagggaccttgaccagtgctatcaatcggcggagctcaaagcaaaagaagcgagggggcaagactggtatagctgtgatcatggaactgcccatcatcaaggccaacgccatcaccaccatcctgatcgccgtgaccttctgcttcgccagcagccagaacatcaccgaggaattctaccagagcacctgcagcgccgtgagcaagggctacctgagcgccctgcggaccggctggtacaccagcgtgatcaccatcgagctgtccaacatcaaagaaaacaagtgcaacggcaccgacgccaaggtgaaactgatcaagcaggaactggacaagtacaagaacgccgtgaccgagctgcagctgctgatgcagagcacccctgccgccaacaaccgggccagacgcgagctgccccggttcatgaactacaccctgaacaacgccaagaaaaccaacgtgaccctgagcaagaagcggaagcggcggttcctgggcttcctgctgggcgtgggcagcgccatcgccagcggcatcgccgtgtccaaggtgctgcacctggaaggcgaggtgaacaagatcaagtccgccctgctgtccaccaacaaggccgtggtgtccctgagcaacggcgtgagcgtgctgaccagcaaggtgctggatctgaagaactacatcgacaagcagctgctgcccatcgtgaacaagcagagctgcagcatcagcaacatcgagaccgtgatcgagttccagcagaagaacaaccggctgctggaaatcacccgggagttcagcgtgaacgccggcgtgaccacccccgtgagcacctacatgctgaccaacagcgagctgctgtccctgatcaatgacatgcccatcaccaacgaccagaagaaactgatgagcaacaacgtgcagatcgtgcggcagcagagctactccatcatgagcatcatcaaagaagaggtgctggcctacgtggtgcagctgcccctgtacggcgtgatcgacaccccctgctggaagctgcacaccagccccctgtgcaccaccaacaccaaagagggcagcaacatctgcctgacccggaccgaccggggctggtactgcaacaacgccggcagcgtgagcttcttccccctggccgacacctgcaaggtgcagagcaaccgggtgttctgcgacaccatgaacagcctgaccctgccctccgaggtgaacctgtgcaacatcgacatcttcaaccccaagtacgactgcaagatcatgacctccaagaccgacgtgagcagctccgtgatcacctccctgggcgccatcgtgagctgctacggcaagaccaagtgcaccgccagcaacaagaaccggggcatcatcaagaccttcagcaacggctgcgactacgtgagcaacaagggcgtggacaccgtgagcgtgggcaacacactgtactacgtgaataagcaggaaggcaagagcctgtacgtgaagggcgagcctatcatcaacttctacgaccccctggtgttccccagcgacgagttcgacgccagcatcagccaggtgaacgagaagatcaaccagagcctggccttcatccggaagagcgacgagctgctgcacaatgtgaatgccggcaagagcaccaccaatatcatgaattttgatctgctcaaacttgcaggcgatgtagaatcaaatcctggacccgcccgggacaggtccatagctctcacgtttctcgcagttggaggagttctgctcttcctctccgtgaacgtgcacgctgacactgggtgtgccatagacatcagccggcaagagctgagatgtggaagtggagtgttcatacacaatgatgtggaggcttggatggaccggtacaagtattaccctgaaacgccacaaggcctagccaagatcattcagaaagctcataaggaaggagtgtgcggtctacgatcagtttccagactggagcatcaaatgtgggaagcagtgaaggacgagctgaacactcttttgaaggagaatggtgtggaccttagtgtcgtggttgagaaacaagggggaatgtacaagtcagcacctaaacgcctcaccgccaccacggaaaaattggaaattggctggaaggcctggggaaagagtattttgtttgcaccagaactcgccaacaacacctttgtggttgatggtccggagaccaaggaatgtccgactcagaatcgcgcttggaatagcttagaagtggaggattttggatttggtctcaccagcactcggatgttcctgaaggtcagagagagcaacacaactgaatgtgactcgaagatcattggaacggctgtcaagaacaacttggcgatccacagtgacctgtcctattggattgaaagcaggctcaatgatacgtggaagcttgaaagggcagttctgggtgaagtcaaatcatgtacgtggcctgagacgcataccttgtggggcgatggaatccttgagagtgacttgataataccagtcacactggcgggaccacgaagcaatcacaatcggagacctgggtataagacacaaaaccagggcccatgggacgaaggccgggtagagattgacttcgattactgcccaggaactacggtcaccctgagtgagagctgcggacaccgtggacctgccactcgcaccaccacagagagcggaaagttgataacagattggtgctgcaggagctgcaccttaccaccactgcgctaccaaactgacagcggctgttggtatggtatggagatcagaccacagagacatgatgaaaagaccctcgtgcagtcacaagtgaatgcttataatgctgatatgattgacccttttcagttgggccttctggtcgtgttcttggccacccaggaggtccttcgcaagaggtggacagccaagatcagcatgccagctatactgattgctctgctagtcctggtgtttgggggcattacttacactgatgtgttacgctatgtcatcttggtgggggcagctttcgcagaatctaattcgggaggagacgtggtacacttggcgctcatggcgaccttcaagatacaaccagtgtttatggtggcatcgtttcttaaagcgagatggaccaaccaggagaacattttgttgatgttggcggctgttttctttcaaatggcttatcacgatgcccgccaaattctgctctgggagatccctgatgtgttgaattcactggcaatagcttggatgatactgagagccataacattcacaacgacatcaaacgtggttgttccgctgctagccctgctaacacccgggctgagatgcttgaatctggatgtgtacaggatactgctgttgatggtcggaataggcagcttgatcagggagaagaggagcgcagctgcaaaaaagaaaggagcaagtctgctatgcttggctctagcctcaacaggactcttcaaccccatgatccttgctgctggactgattgcatgtgatcccaaccgtaaacgcgggtggcccgcaactgaagtgatgacagctgtcggcctaatgtttgccatcgtcggagggctggcagagcttgacattgactccatggccattccaatgactatcgcggggctcatgtttgctgctttcgtgatttctgggaaatcaacagatatgtggattgagagaacggcggacatttcctgggaaagtgatgcagagattacaggctcgagcgaaagagttgatgtgcggcttgatgatgatggaaacttccagctcatgaatgatccaggagcaccttggaagatatggatgctcagaatggtctgtctcgcgattagtgcgtacaccccctgggcaatcttgccctcagtagttggattttggataactctccaatacacaaagagaggaggcgtgttgtgggacactccctcaccaaaggagtacaaaaagggggacacgaccaccggcgtctacaggatcatgactcgtgggctgctcggcagttatcaagcaggagcaggcgtgatggttgaaggtgttttccacaccctttggcatacaacaaaaggagccgctttgatgagcggagagggccgcctggacccatactggggcagtgtcaaggaggatcgactttgttacggaggaccctggaaattgcagcacaagtggaacgggcaggatgaggtgcagatgattgtggtggaacctggcaagaacgttaagaacgtccagacgaaaccaggggtgttcaaaacacctgaaggagaaatcggggccgtgactttggacttccccactggaacatcaggctcaccaatagtggacaaaaacggtgatgtgattgggctttatggcaatggagtcataatgcccaacggctcatacataagcgcgatagtgcagggtgaaaggatggatgagccaatcccagccggattcgaacctgagatgctgaggaaaaaacagatcactgtactggatctccatcccggcgccggtaaaacaaggaggattctgccacagatcatcaaagaggccataaacagaagactgagaacagccgtgctagcaccaaccagggttgtggctgctgagatggctgaagcactgagaggactgcccatccggtaccagacatccgcagtgcccagagaacataatggaaatgagattgttgatgtcatgtgtcatgctaccctcacccacaggctgatgtctcctcacagggtgccgaactacaacctgttcgtgatggatgaggctcatttcaccgacccagctagcattgcagcaagaggttacatttccacaaaggtcgagctaggggaggcggcggcaatattcatgacagccaccccaccaggcacttcagatccattcccagagtccaattcaccaatttccgacttacagactgagatcccggatcgagcttggaactctggatacgaatggatcacagaatacaccgggaagacggtttggtttgtgcctagtgttaagatggggaatgagattgccctttgcctacaacgtgctggaaagaaagtagtccaattgaacagaaagtcgtacgagacggagtacccaaaatgtaagaacgatgattgggactttgttatcacaacagacatatctgaaatgggggctaacttcaaggcgagcagggtgattgacagccggaagagtgtgaaaccaaccatcataacagaaggagaagggagagtgatcctgggagaaccatctgcagtgacagcagctagtgccgcccagagacgtggacgtatcggtagaaatccgtcgcaagttggtgatgagtactgttatggggggcacacgaatgaagacgactcgaacttcgcccattggactgaggcacgaatcatgctggacaacatcaacatgccaaacggactgatcgctcaattctaccaaccagagcgtgagaaggtatataccatggatggggaataccggctcagaggagaagagagaaaaaactttctggaactgttgaggactgcagatctgccagtttggctggcttacaaggttgcagcggctggagtgtcataccacgaccggaggtggtgctttgatggtcctaggacaaacacaattttagaagacaacaacgaagtggaagtcatcacgaagcttggtgaaaggaagattctgaggccgcgctggattgacgccagggtgtactcggatcaccaggcactaaaggcgttcaaggacttcgcctcgggaaaacgttctcagatagggctcattgaggttctgggaaagatgcctgagcacttcatggggaagacatgggaagcacttgacaccatgtacgttgtggccactgcagagaaaggaggaagagctcacagaatggccctggaggaactgccagatgctcttcagacaattgccttgattgccttattgagtgtgatgaccatgggagtattcttcctcctcatgcagcggaagggcattggaaagataggtttgggaggcgctgtcttgggagtcgcgacctttttctgttggatggctgaagttccaggaacgaagatcgccggaatgttgctgctctcccttctcttgatgattgtgctaattcctgagccagagaagcaacgttcgcagacagacaaccagctagccgtgttcctgatatgtgtcatgacccttgtgagcgcagtggcagccaacgagatgggttggctagataagaccaagagtgacataagcagtttgtttgggcaaagaattgaggtcaaggagaatttcagcatgggagagtttcttctggacttgaggccggcaacagcctggtcactgtacgctgtgacaacagcggtcctcactccactgctaaagcatttgatcacgtcagattacatcaacacctcattgacctcaataaacgttcaggcaagtgcactattcacactcgcgcgaggcttccccttcgtcgatgttggagtgtcggctctcctgctagcagccggatgctggggacaagtcaccctcaccgttacggtaacagcggcaacactccttttttgccactatgcctacatggttcccggttggcaagctgaggcaatgcgctcagcccagcggcggacagcggccggaatcatgaagaacgctgtagtggatggcatcgtggccacggacgtcccagaattagagcgcaccacacccatcatgcagaagaaaattggacagatcatgctgatcttggtgtctctagctgcagtagtagtgaacccgtctgtgaagacagtacgagaagccggaattttgatcacggccgcagcggtgacgctttgggagaatggagcaagctctgtttggaacgcaacaactgccatcggactctgccacatcatgcgtgggggttggttgtcatgtctatccataacatggacactcataaagaacatggaaaaaccaggactaaaaagaggtggggcaaaaggacgcaccttgggagaggtttggaaagaaagactcaaccagatgacaaaagaagagttcactaggtaccgcaaagaggccatcatcgaagtcgatcgctcagcggcaaaacacgccaggaaagaaggcaatgtcactggagggcatccagtctctaggggcacagcaaaactgagatggctggtcgaacggaggtttctcgaaccggtcggaaaagtgattgaccttggatgtggaagaggcggttggtgttactatatggcaacccaaaaaagagtccaagaagtcagagggtacacaaagggcggtcccggacatgaagagccccaactagtgcaaagttatggatggaacattgtcaccatgaagagtggagtggatgtgttctacagaccttctgagtgttgtgacaccctcctttgtgacatcggagagtcctcgtcaagtgctgaggttgaagagcataggacgattcgggtccttgaaatggttgaggactggctgcaccgagggccaagggaattttgcgtgaaggtgctctgcccctacatgccgaaagtcatagagaagatggagctgctccaacgccggtatggggggggactggtcagaaacccactctcacggaattccacgcacgagatgtattgggtgagtcgagcttcaggcaatgtggtacattcagtgaatatgaccagccaggtgctcctaggaagaatggaaaaaaggacctggaagggaccccaatacgaggaagacgtaaacttgggaagtggaaccagggcggtgggaaaacccctgctcaactcagacaccagtaaaatcaagaacaggattgaacgactcaggcgtgagtacagttcgacgtggcaccacgatgagaaccacccatatagaacctggaactatcatggcagttatgatgtgaagcccacaggctccgccagttcgctggtcaatggagtggtcaggctcctctcaaaaccatgggacaccatcacgaatgttaccaccatggccatgactgacactactcccttcgggcagcagcgagtgttcaaagagaaggtggacacgaaagctcctgaaccgccagaaggagtgaagtacgtgctcaacgagaccaccaactggttgtgggcgtttttggccagagaaaaacgtcccagaatgtgctctcgagaggaattcataagaaaggtcaacagcaatgcagctttgggtgccatgtttgaagagcagaatcaatggaggagcgccagagaagcagttgaagatccaaaattttgggaaatggtggatgaggagcgcgaggcacatctgcggggggaatgtcacacttgcatttacaacatgatgggaaagagagagaaaaaacccggagagttcggaaaggccaagggaagcagagccatttggttcatgtggctcggagctcgctttctggagttcgaggctctgggttttctcaatgaagaccactggcttggaagaaagaactcaggaggaggtgtcgagggcttgggcctccaaaaactgggttacatcctgcgtgaagttggcatccggcctgggggcaagatctatgctgatgacacagctggctgggacacccgcatcacgagagctgacttggaaaatgaagctaaggtgcttgagctgcttgatggggaacatcggcgtcttgccagggccatcattgagctcacctatcgtcacaaagttgtgaaagtgatgcgcccggctgctgatggaagaaccgttatggatgttatctccagagaagatcagagggggagtggacaagttgtcacctacgccctaaacactttcaccaacctggctgtccagctggtgaggatgatggaaggggaaggagtgattggcccagatgatgtggagaaactcacaaaagggaaaggacccaaagtcaggacctggctgtttgagaatggggaagaaagactcagccgcatggctgtcagtggagatgactgtgtggtaaagcccctggacgatcgctttgccacctcgctccacttcctcaatgctatgtcaaaggttcgcaaagacatccaagagtggaaaccgtcaactggatggtatgattggcagcaggttccattttgctcaaaccatttcactgaattgatcatgaaagatggaagaacactggtggttccatgccgaggacaggatgaattggtaggcagagctcgcatatctccaggggccggatggaacgtccgcgacactgcttgtctggctaagtcttatgcccagatgtggctgcttctgtacttccacagaagagacctgcggctcatggccaacgccatttgctccgctgtccctgtgaattgggtccctaccggaagaaccacgtggtccatccatgcaggaggagagtggatgacaacagaggacatgttggaggtctggaaccgtgtttggatagaggagaatgaatggatggaagacaaaaccccagtggagaaatggagtgacgtcccatattcaggaaaacgagaggacatctggtgtggcagcctgattggcacaagagcccgagccacgtgggcagaaaacatccaggtggctatcaaccaagtcagagcaatcatcggagatgagaagtatgtggattacatgagttcactaaagagatatgaagacacaactttggttgaggacacagtactgtagatatttaatcaattgtaaatagacaatataagtatgcataaaagtgtagttttatagtagtatttagtggtgttagtgtaaatagttaagaaaatcttgaggagaaagtcaggccgggaagttcccgccaccggaagttgagtagacggtgctgcctgcgactcaaccccaggaggactgggtgaacaaagccgcgaagtgatccatgtaagccctcagaaccgtctcggaaggaggaccccacatgttgtaacttcaaagcccaatgtcagaccacgctacggcgtgctactctgcggagagtgcagtctgcgatagtgccccaggaggactgggttaacaaaggcaaaccaacgccccacgcggcccaagccccggtaatggtgttaaccagggcgaaaggactagaggttagaggagaccccgcggtttaaagtgcacggcccagcctggctgaagctgtaggtcaggggaaggactagaggttagtggagaccccgtgccacaaaacaccacaacaaaacagcaaatagacacctgggatagactaggagatcttctgctctgcacaaccagccacacggcacagtgcgccgacaatggtggctggtggtgcgagaacacaggatct

SEQ ID NO: 84 - DNA, 10705 bp, Artificial Sequence
  Synthetic Construct
tcatcaagcggacacactcgactgtttgaatcatcacaaacactcctaattgttgttaattgtgtcacgctcgacaaagaatcgtgcttctagagctacagattctttggtcctcccgggccgttctcggcccgacagataaacgattttgcgccttacggggcgcacaacaggaactaacctgaattctcccgatacaactcggactagctgccgttccccggttatgctaaacacaaccgagagaaccgcaagaagtccaagtgtcgttaacgaggctgggctcgtcacgacctagctacctctccacacttgtttgtttgtcgctactttgtggaagactcaaagttcttccttgatccctggaactggtcacgatagttagccgcctcgagtttcgttttcttcgctcccccgttctgaccatatcgacactagtaccttgacgggtagtagttccggttgcggtagtggtggtaggactagcggcactggaagacgaagcggtcgtcggtcttgtagtggctccttaagatggtctcgtggacgtcgcggcactcgttcccgatggactcgcgggacgcctggccgaccatgtggtcgcactagtggtagctcgacaggttgtagtttcttttgttcacgttgccgtggctgcggttccactttgactagttcgtccttgacctgttcatgttcttgcggcactggctcgacgtcgacgactacgtctcgtggggacggcggttgttggcccggtctgcgctcgacggggccaagtacttgatgtgggacttgttgcggttcttttggttgcactgggactcgttcttcgccttcgccgccaaggacccgaaggacgacccgcacccgtcgcggtagcggtcgccgtagcggcacaggttccacgacgtggaccttccgctccacttgttctagttcaggcgggacgacaggtggttgttccggcaccacagggactcgttgccgcactcgcacgactggtcgttccacgacctagacttcttgatgtagctgttcgtcgacgacgggtagcacttgttcgtctcgacgtcgtagtcgttgtagctctggcactagctcaaggtcgtcttcttgttggccgacgacctttagtgggccctcaagtcgcacttgcggccgcactggtgggggcactcgtggatgtacgactggttgtcgctcgacgacagggactagttactgtacgggtagtggttgctggtcttctttgactactcgttgttgcacgtctagcacgccgtcgtctcgatgaggtagtactcgtagtagtttcttctccacgaccggatgcaccacgtcgacggggacatgccgcactagctgtgggggacgaccttcgacgtgtggtcgggggacacgtggtggttgtggtttctcccgtcgttgtagacggactgggcctggctggccccgaccatgacgttgttgcggccgtcgcactcgaagaagggggaccggctgtggacgttccacgtctcgttggcccacaagacgctgtggtacttgtcggactgggacgggaggctccacttggacacgttgtagctgtagaagttggggttcatgctgacgttctagtactggaggttctggctgcactcgtcgaggcactagtggagggacccgcggtagcactcgacgatgccgttctggttcacgtggcggtcgttgttcttggccccgtagtagttctggaagtcgttgccgacgctgatgcactcgttgttcccgcacctgtggcactcgcacccgttgtgtgacatgatgcacttattcgtccttccgttctcggacatgcacttcccgctcggatagtagttgaagatgctgggggaccacaaggggtcgctgctcaagctgcggtcgtagtcggtccacttgctcttctagttggtctcggaccggaagtaggccttctcgctgctcgacgacgtgttacacttacggccgttctcgtggtggttatagtacttaaaactagacgagtttgaacgtccgctacatcttagtttaggacctgggcgggccctgtccaggtatcgagagtgcaaagagcgtcaacctcctcaagacgagaaggagaggcacttgcacgtgcgactgtgacccacacggtatctgtagtcggccgttctcgactctacaccttcacctcacaagtatgtgttactacacctccgaacctacctggccatgttcataatgggactttgcggtgttccggatcggttctagtaagtctttcgagtattccttcctcacacgccagatgctagtcaaaggtctgacctcgtagtttacacccttcgtcacttcctgctcgacttgtgagaaaacttcctcttaccacacctggaatcacagcaccaactctttgttcccccttacatgttcagtcgtggatttgcggagtggcggtggtgcctttttaacctttaaccgaccttccggacccctttctcataaaacaaacgtggtcttgagcggttgttgtggaaacaccaactaccaggcctctggttccttacaggctgagtcttagcgcgaaccttatcgaatcttcacctcctaaaacctaaaccagagtggtcgtgagcctacaaggacttccagtctctctcgttgtgttgacttacactgagcttctagtaaccttgccgacagttcttgttgaaccgctaggtgtcactggacaggataacctaactttcgtccgagttactatgcaccttcgaactttcccgtcaagacccacttcagtttagtacatgcaccggactctgcgtatggaacaccccgctaccttaggaactctcactgaactattatggtcagtgtgaccgccctggtgcttcgttagtgttagcctctggacccatattctgtgttttggtcccgggtaccctgcttccggcccatctctaactgaagctaatgacgggtccttgatgccagtgggactcactctcgacgcctgtggcacctggacggtgagcgtggtggtgtctctcgcctttcaactattgtctaaccacgacgtcctcgacgtggaatggtggtgacgcgatggtttgactgtcgccgacaaccataccatacctctagtctggtgtctctgtactacttttctgggagcacgtcagtgttcacttacgaatattacgactatactaactgggaaaagtcaacccggaagaccagcacaagaaccggtgggtcctccaggaagcgttctccacctgtcggttctagtcgtacggtcgatatgactaacgagacgatcaggaccacaaacccccgtaatgaatgtgactacacaatgcgatacagtagaaccacccccgtcgaaagcgtcttagattaagccctcctctgcaccatgtgaaccgcgagtaccgctggaagttctatgttggtcacaaataccaccgtagcaaagaatttcgctctacctggttggtcctcttgtaaaacaactacaaccgccgacaaaagaaagtttaccgaatagtgctacgggcggtttaagacgagaccctctagggactacacaacttaagtgaccgttatcgaacctactatgactctcggtattgtaagtgttgctgtagtttgcaccaacaaggcgacgatcgggacgattgtgggcccgactctacgaacttagacctacacatgtcctatgacgacaactaccagccttatccgtcgaactagtccctcttctcctcgcgtcgacgttttttctttcctcgttcagacgatacgaaccgagatcggagttgtcctgagaagttggggtactaggaacgacgacctgactaacgtacactagggttggcatttgcgcccaccgggcgttgacttcactactgtcgacagccggattacaaacggtagcagcctcccgaccgtctcgaactgtaactgaggtaccggtaaggttactgatagcgccccgagtacaaacgacgaaagcactaaagaccctttagttgtctatacacctaactctcttgccgcctgtaaaggaccctttcactacgtctctaatgtccgagctcgctttctcaactacacgccgaactactactacctttgaaggtcgagtacttactaggtcctcgtggaaccttctatacctacgagtcttaccagacagagcgctaatcacgcatgtgggggacccgttagaacgggagtcatcaacctaaaacctattgagaggttatgtgtttctctcctccgcacaacaccctgtgagggagtggtttcctcatgtttttccccctgtgctggtggccgcagatgtcctagtactgagcacccgacgagccgtcaatagttcgtcctcgtccgcactaccaacttccacaaaaggtgtgggaaaccgtatgttgttttcctcggcgaaactactcgcctctcccggcggacctgggtatgaccccgtcacagttcctcctagctgaaacaatgcctcctgggacctttaacgtcgtgttcaccttgcccgtcctactccacgtctactaacaccaccttggaccgttcttgcaattcttgcaggtctgctttggtccccacaagttttgtggacttcctctttagccccggcactgaaacctgaaggggtgaccttgtagtccgagtggttatcacctgtttttgccactacactaacccgaaataccgttacctcagtattacgggttgccgagtatgtattcgcgctatcacgtcccactttcctacctactcggttagggtcggcctaagcttggactctacgactccttttttgtctagtgacatgacctagaggtagggccgcggccattttgttcctcctaagacggtgtctagtagtttctccggtatttgtcttctgactcttgtcggcacgatcgtggttggtcccaacaccgacgactctaccgacttcgtgactctcctgacgggtaggccatggtctgtaggcgtcacgggtctcttgtattacctttactctaacaactacagtacacagtacgatgggagtgggtgtccgactacagaggagtgtcccacggcttgatgttggacaagcactacctactccgagtaaagtggctgggtcgatcgtaacgtcgttctccaatgtaaaggtgtttccagctcgatcccctccgccgccgttataagtactgtcggtggggtggtccgtgaagtctaggtaagggtctcaggttaagtggttaaaggctgaatgtctgactctagggcctagctcgaaccttgagacctatgcttacctagtgtcttatgtggcccttctgccaaaccaaacacggatcacaattctaccccttactctaacgggaaacggatgttgcacgacctttctttcatcaggttaacttgtctttcagcatgctctgcctcatgggttttacattcttgctactaaccctgaaacaatagtgttgtctgtatagactttacccccgattgaagttccgctcgtcccactaactgtcggccttctcacactttggttggtagtattgtcttcctcttccctctcactaggaccctcttggtagacgtcactgtcgtcgatcacggcgggtctctgcacctgcatagccatctttaggcagcgttcaaccactactcatgacaataccccccgtgtgcttacttctgctgagcttgaagcgggtaacctgactccgtgcttagtacgacctgttgtagttgtacggtttgcctgactagcgagttaagatggttggtctcgcactcttccatatatggtacctaccccttatggccgagtctcctcttctctcttttttgaaagaccttgacaactcctgacgtctagacggtcaaaccgaccgaatgttccaacgtcgccgacctcacagtatggtgctggcctccaccacgaaactaccaggatcctgtttgtgttaaaatcttctgttgttgcttcaccttcagtagtgcttcgaaccactttccttctaagactccggcgcgacctaactgcggtcccacatgagcctagtggtccgtgatttccgcaagttcctgaagcggagcccttttgcaagagtctatcccgagtaactccaagaccctttctacggactcgtgaagtaccccttctgtacccttcgtgaactgtggtacatgcaacaccggtgacgtctctttcctccttctcgagtgtcttaccgggacctccttgacggtctacgagaagtctgttaacggaactaacggaataactcacactactggtaccctcataagaaggaggagtacgtcgccttcccgtaacctttctatccaaaccctccgcgacagaaccctcagcgctggaaaaagacaacctaccgacttcaaggtccttgcttctagcggccttacaacgacgagagggaagagaactactaacacgattaaggactcggtctcttcgttgcaagcgtctgtctgttggtcgatcggcacaaggactatacacagtactgggaacactcgcgtcaccgtcggttgctctacccaaccgatctattctggttctcactgtattcgtcaaacaaacccgtttcttaactccagttcctcttaaagtcgtaccctctcaaagaagacctgaactccggccgttgtcggaccagtgacatgcgacactgttgtcgccaggagtgaggtgacgatttcgtaaactagtgcagtctaatgtagttgtggagtaactggagttatttgcaagtccgttcacgtgataagtgtgagcgcgctccgaaggggaagcagctacaacctcacagccgagaggacgatcgtcggcctacgacccctgttcagtgggagtggcaatgccattgtcgccgttgtgaggaaaaaacggtgatacggatgtaccaagggccaaccgttcgactccgttacgcgagtcgggtcgccgcctgtcgccggccttagtacttcttgcgacatcacctaccgtagcaccggtgcctgcagggtcttaatctcgcgtggtgtgggtagtacgtcttcttttaacctgtctagtacgactagaaccacagagatcgacgtcatcatcacttgggcagacacttctgtcatgctcttcggccttaaaactagtgccggcgtcgccactgcgaaaccctcttacctcgttcgagacaaaccttgcgttgttgacggtagcctgagacggtgtagtacgcacccccaaccaacagtacagataggtattgtacctgtgagtatttcttgtacctttttggtcctgatttttctccaccccgttttcctgcgtggaaccctctccaaacctttctttctgagttggtctactgttttcttctcaagtgatccatggcgtttctccggtagtagcttcagctagcgagtcgccgttttgtgcggtcctttcttccgttacagtgacctcccgtaggtcagagatccccgtgtcgttttgactctaccgaccagcttgcctccaaagagcttggccagccttttcactaactggaacctacaccttctccgccaaccacaatgatataccgttgggttttttctcaggttcttcagtctcccatgtgtttcccgccagggcctgtacttctcggggttgatcacgtttcaatacctaccttgtaacagtggtacttctcacctcacctacacaagatgtctggaagactcacaacactgtgggaggaaacactgtagcctctcaggagcagttcacgactccaacttctcgtatcctgctaagcccaggaactttaccaactcctgaccgacgtggctcccggttcccttaaaacgcacttccacgagacggggatgtacggctttcagtatctcttctacctcgacgaggttgcggccatacccccccctgaccagtctttgggtgagagtgccttaaggtgcgtgctctacataacccactcagctcgaagtccgttacaccatgtaagtcacttatactggtcggtccacgaggatccttcttaccttttttcctggaccttccctggggttatgctccttctgcatttgaacccttcaccttggtcccgccacccttttggggacgagttgagtctgtggtcattttagttcttgtcctaacttgctgagtccgcactcatgtcaagctgcaccgtggtgctactcttggtgggtatatcttggaccttgatagtaccgtcaatactacacttcgggtgtccgaggcggtcaagcgaccagttacctcaccagtccgaggagagttttggtaccctgtggtagtgcttacaatggtggtaccggtactgactgtgatgagggaagcccgtcgtcgctcacaagtttctcttccacctgtgctttcgaggacttggcggtcttcctcacttcatgcacgagttgctctggtggttgaccaacacccgcaaaaaccggtctctttttgcagggtcttacacgagagctctccttaagtattctttccagttgtcgttacgtcgaaacccacggtacaaacttctcgtcttagttacctcctcgcggtctcttcgtcaacttctaggttttaaaaccctttaccacctactcctcgcgctccgtgtagacgccccccttacagtgtgaacgtaaatgttgtactaccctttctctctcttttttgggcctctcaagcctttccggttcccttcgtctcggtaaaccaagtacaccgagcctcgagcgaaagacctcaagctccgagacccaaaagagttacttctggtgaccgaaccttctttcttgagtcctcctccacagctcccgaacccggaggtttttgacccaatgtaggacgcacttcaaccgtaggccggacccccgttctagatacgactactgtgtcgaccgaccctgtgggcgtagtgctctcgactgaaccttttacttcgattccacgaactcgacgaactaccccttgtagccgcagaacggtcccggtagtaactcgagtggatagcagtgtttcaacactttcactacgcgggccgacgactaccttcttggcaatacctacaatagaggtctcttctagtctccccctcacctgttcaacagtggatgcgggatttgtgaaagtggttggaccgacaggtcgaccactcctactaccttccccttcctcactaaccgggtctactacacctctttgagtgttttccctttcctgggtttcagtcctggaccgacaaactcttaccccttctttctgagtcggcgtaccgacagtcacctctactgacacaccatttcggggacctgctagcgaaacggtggagcgaggtgaaggagttacgatacagtttccaagcgtttctgtaggttctcacctttggcagttgacctaccatactaaccgtcgtccaaggtaaaacgagtttggtaaagtgacttaactagtactttctaccttcttgtgaccaccaaggtacggctcctgtcctacttaaccatccgtctcgagcgtatagaggtccccggcctaccttgcaggcgctgtgacgaacagaccgattcagaatacgggtctacaccgacgaagacatgaaggtgtcttctctggacgccgagtaccggttgcggtaaacgaggcgacagggacacttaacccagggatggccttcttggtgcaccaggtaggtacgtcctcctctcacctactgttgtctcctgtacaacctccagaccttggcacaaacctatctcctcttacttacctaccttctgttttggggtcacctctttacctcactgcagggtataagtccttttgctctcctgtagaccacaccgtcggactaaccgtgttctcgggctcggtgcacccgtcttttgtaggtccaccgatagttggttcagtctcgttagtagcctctactcttcatacacctaatgtactcaagtgatttctctatacttctgtgttgaaaccaactcctgtgtcatgacatctataaattagttaacatttatctgttatattcatacgtattttcacatcaaaatatcatcataaatcaccacaatcacatttatcaattcttttagaactcctctttcagtccggcccttcaagggcggtggccttcaactcatctgccacgacggacgctgagttggggtcctcctgacccacttgtttcggcgcttcactaggtacattcgggagtcttggcagagccttcctcctggggtgtacaacattgaagtttcgggttacagtctggtgcgatgccgcacgatgagacgcctctcacgtcagacgctatcacggggtcctcctgacccaattgtttccgtttggttgcggggtgcgccgggttcggggccattaccacaattggtcccgctttcctgatctccaatctcctctggggcgccaaatttcacgtgccgggtcggaccgacttcgacatccagtccccttcctgatctccaatcacctctggggcacggtgttttgtggtgttgttttgtcgtttatctgtggaccctatctgatcctctagaagacgagacgtgttggtcggtgtgccgtgtcacgcggctgttaccaccgaccaccacgctcttgtgtcctaga

SEQ ID NO: 85 - protein, 3915 aa, Artificial Sequence
  Synthetic Construct
MSKKPGGPGKSRAVNMLKRGMPRVLSLIGLKQKKRGGKTGIAVIMELPIIKANAITTILIAVTFCFASSQNITEEFYQSTCSAVSKGYLSALRTGWYTSVITIELSNIKENKCNGTDAKVKLIKQELDKYKNAVTELQLLMQSTPAANNRARRELPRFMNYTLNNAKKTNVTLSKKRKRRFLGFLLGVGSAIASGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCNNAGSVSFFPLADTCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMNFDLLKLAGDVESNPGPGGKTGIAVMIGLIACVGAVTLSNFQGKVMMTVNATDVTDVITIPTAAGKNLCIVRAMDVGYMCDDTITYECPVLSAGNDPEDIDCWCTKSAVYVRYGRCTKTRHSRRSRRSLTVQTHGESTLANKKGAWMDSTKATRYLVKTESWILRNPGYALVAAVIGWMLGSNTMQRVVFVVLLLLVAPAYSFNCLGMSNRDFLEGVSGATWVDLVLEGDSCVTIMSKDKPTIDVKMMNMEAANLAEVRSYCYLATVSDLSTKAACPAMGEAHNDKRADPAFVCRQGVVDRGWGNGCGLFGKGSIDTCAKFACSTKAIGRTILKENIKYEVAIFVHGPTTVESHGNYSTQVGATQAGRFSITPAAPSYTLKLGEYGEVTVDCEPRSGIDTNAYYVMTVGTKTFLVHREWFMDLNLPWSSAGSTVWRNRETLMEFEEPHATKQSVIALGSQEGALHQALAGAIPVEFSSNTVKLTSGHLKCRVKMEKLQLKGTTYGVCSKAFKFLGTPADTGHGTVVLELQYTGTDGPCKVPISSVASLNDLTPVGRLVTVNPFVSVATANAKVLIELEPPFGDSYIVVGRGEQQINHHWHKSGSSIGKAFTTTLKGAQRLAALGDTAWDFGSVGGVFTSVGKAVHQVFGGAFRSLFGGMSWITQGLLGALLLWMGINARDRSIALTFLAVGGVLLFLSVNVHADTGCAIDISRQELRCGSGVFIHNDVEAWMDRYKYYPETPQGLAKIIQKAHKEGVCGLRSVSRLEHQMWEAVKDELNTLLKENGVDLSVVVEKQGGMYKSAPKRLTATTEKLEIGWKAWGKSILFAPELANNTFVVDGPETKECPTQNRAWNSLEVEDFGFGLTSTRMFLKVRESNTTECDSKIIGTAVKNNLAIHSDLSYWIESRLNDTWKLERAVLGEVKSCTWPETHTLWGDGILESDLIIPVTLAGPRSNHNRRPGYKTQNQGPWDEGRVEIDFDYCPGTTVTLSESCGHRGPATRTTTESGKLITDWCCRSCTLPPLRYQTDSGCWYGMEIRPQRHDEKTLVQSQVNAYNADMIDPFQLGLLVVFLATQEVLRKRWTAKISMPAILIALLVLVFGGITYTDVLRYVILVGAAFAESNSGGDVVHLALMATFKIQPVFMVASFLKARWTNQENILLMLAAVFFQMAYHDARQILLWEIPDVLNSLAIAWMILRAITFTTTSNVVVPLLALLTPGLRCLNLDVYRILLLMVGIGSLIREKRSAAAKKKGASLLCLALASTGLFNPMILAAGLIACDPNRKRGWPATEVMTAVGLMFAIVGGLAELDIDSMAIPMTIAGLMFAAFVISGKSTDMWIERTADISWESDAEITGSSERVDVRLDDDGNFQLMNDPGAPWKIWMLRMVCLAISAYTPWAILPSVVGFWITLQYTKRGGVLWDTPSPKEYKKGDTTTGVYRIMTRGLLGSYQAGAGVMVEGVFHTLWHTTKGAALMSGEGRLDPYWGSVKEDRLCYGGPWKLQHKWNGQDEVQMIVVEPGKNVKNVQTKPGVFKTPEGEIGAVTLDFPTGTSGSPIVDKNGDVIGLYGNGVIMPNGSYISAIVQGERMDEPIPAGFEPEMLRKKQITVLDLHPGAGKTRRILPQIIKEAINRRLRTAVLAPTRVVAAEMAEALRGLPIRYQTSAVPREHNGNEIVDVMCHATLTHRLMSPHRVPNYNLFVMDEAHFTDPASIAARGYISTKVELGEAAAIFMTATPPGTSDPFPESNSPISDLQTEIPDRAWNSGYEWITEYTGKTVWFVPSVKMGNEIALCLQRAGKKVVQLNRKSYETEYPKCKNDDWDFVITTDISEMGANFKASRVIDSRKSVKPTIITEGEGRVILGEPSAVTAASAAQRRGRIGRNPSQVGDEYCYGGHTNEDDSNFAHWTEARIMLDNINMPNGLIAQFYQPEREKVYTMDGEYRLRGEERKNFLELLRTADLPVWLAYKVAAAGVSYHDRRWCFDGPRTNTILEDNNEVEVITKLGERKILRPRWIDARVYSDHQALKAFKDFASGKRSQIGLIEVLGKMPEHFMGKTWEALDTMYVVATAEKGGRAHRMALEELPDALQTIALIALLSVMTMGVFFLLMQRKGIGKIGLGGAVLGVATFFCWMAEVPGTKIAGMLLLSLLLMIVLIPEPEKQRSQTDNQLAVFLICVMTLVSAVAANEMGWLDKTKSDISSLFGQRIEVKENFSMGEFLLDLRPATAWSLYAVTTAVLTPLLKHLITSDYINTSLTSINVQASALFTLARGFPFVDVGVSALLLAAGCWGQVTLTVTVTAATLLFCHYAYMVPGWQAEAMRSAQRRTAAGIMKNAVVDGIVATDVPELERTTPIMQKKIGQIMLILVSLAAVVVNPSVKTVREAGILITAAAVTLWENGASSVWNATTAIGLCHIMRGGWLSCLSITWTLIKNMEKPGLKRGGAKGRTLGEVWKERLNQMTKEEFTRYRKEAIIEVDRSAAKHARKEGNVTGGHPVSRGTAKLRWLVERRFLEPVGKVIDLGCGRGGWCYYMATQKRVQEVRGYTKGGPGHEEPQLVQSYGWNIVTMKSGVDVFYRPSECCDTLLCDIGESSSSAEVEEHRTIRVLEMVEDWLHRGPREFCVKVLCPYMPKVIEKMELLQRRYGGGLVRNPLSRNSTHEMYWVSRASGNVVHSVNMTSQVLLGRMEKRTWKGPQYEEDVNLGSGTRAVGKPLLNSDTSKIKNRIERLRREYSSTWHHDENHPYRTWNYHGSYDVKPTGSASSLVNGVVRLLSKPWDTITNVTTMAMTDTTPFGQQRVFKEKVDTKAPEPPEGVKYVLNETTNWLWAFLAREKRPRMCSREEFIRKVNSNAALGAMFEEQNQWRSAREAVEDPKFWEMVDEEREAHLRGECHTCIYNMMGKREKKPGEFGKAKGSRAIWFMWLGARFLEFEALGFLNEDHWLGRKNSGGGVEGLGLQKLGYILREVGIRPGGKIYADDTAGWDTRITRADLENEAKVLELLDGEHRRLARAIIELTYRHKVVKVMRPAADGRTVMDVISREDQRGSGQVVTYALNTFTNLAVQLVRMMEGEGVIGPDDVEKLTKGKGPKVRTWLFENGEERLSRMAVSGDDCVVKPLDDRFATSLHFLNAMSKVRKDIQEWKPSTGWYDWQQVPFCSNHFTELIMKDGRTLVVPCRGQDELVGRARISPGAGWNVRDTACLAKSYAQMWLLLYFHRRDLRLMANAICSAVPVNWVPTGRTTWSIHAGGEWMTTEDMLEVWNRVWIEENEWMEDKTPVEKWSDVPYSGKREDIWCGSLIGTRARATWAENIQVAINQVRAIIGDEKYVDYMSSLKRYEDTTLVEDTVL

SEQ ID NO: 86 - DNA, 12475 bp, Artificial Sequence
  Synthetic Construct
agtagttcgcctgtgtgagctgacaaacttagtagtgtttgtgaggattaacaacaattaacacagtgcgagctgtttcttagcacgaagatctcgatgtctaagaaaccaggagggcccggcaagagccgggctgtcaatatgctaaaacgcggaatgccccgcgtgttgtccttgattggacttaagcaaaagaagcgagggggcaagactggtatagctgtgatcatggaactgcccatcatcaaggccaacgccatcaccaccatcctgatcgccgtgaccttctgcttcgccagcagccagaacatcaccgaggaattctaccagagcacctgcagcgccgtgagcaagggctacctgagcgccctgcggaccggctggtacaccagcgtgatcaccatcgagctgtccaacatcaaagaaaacaagtgcaacggcaccgacgccaaggtgaaactgatcaagcaggaactggacaagtacaagaacgccgtgaccgagctgcagctgctgatgcagagcacccctgccgccaacaaccgggccagacgcgagctgccccggttcatgaactacaccctgaacaacgccaagaaaaccaacgtgaccctgagcaagaagcggaagcggcggttcctgggcttcctgctgggcgtgggcagcgccatcgccagcggcatcgccgtgtccaaggtgctgcacctggaaggcgaggtgaacaagatcaagtccgccctgctgtccaccaacaaggccgtggtgtccctgagcaacggcgtgagcgtgctgaccagcaaggtgctggatctgaagaactacatcgacaagcagctgctgcccatcgtgaacaagcagagctgcagcatcagcaacatcgagaccgtgatcgagttccagcagaagaacaaccggctgctggaaatcacccgggagttcagcgtgaacgccggcgtgaccacccccgtgagcacctacatgctgaccaacagcgagctgctgtccctgatcaatgacatgcccatcaccaacgaccagaagaaactgatgagcaacaacgtgcagatcgtgcggcagcagagctactccatcatgagcatcatcaaagaagaggtgctggcctacgtggtgcagctgcccctgtacggcgtgatcgacaccccctgctggaagctgcacaccagccccctgtgcaccaccaacaccaaagagggcagcaacatctgcctgacccggaccgaccggggctggtactgcaacaacgccggcagcgtgagcttcttccccctggccgacacctgcaaggtgcagagcaaccgggtgttctgcgacaccatgaacagcctgaccctgccctccgaggtgaacctgtgcaacatcgacatcttcaaccccaagtacgactgcaagatcatgacctccaagaccgacgtgagcagctccgtgatcacctccctgggcgccatcgtgagctgctacggcaagaccaagtgcaccgccagcaacaagaaccggggcatcatcaagaccttcagcaacggctgcgactacgtgagcaacaagggcgtggacaccgtgagcgtgggcaacacactgtactacgtgaataagcaggaaggcaagagcctgtacgtgaagggcgagcctatcatcaacttctacgaccccctggtgttccccagcgacgagttcgacgccagcatcagccaggtgaacgagaagatcaaccagagcctggccttcatccggaagagcgacgagctgctgcacaatgtgaatgccggcaagagcaccaccaatatcatgaattttgatctgctcaaacttgcaggcgatgtagaatcaaatcctggacccggaggaaagaccggtattgcagtcatgattggcctgatcgcctgcgtaggagcagttaccctctctaacttccaagggaaggtgatgatgacggtaaatgctactgacgtcacagatgtcatcacgattccaacagctgctggaaagaacctatgcattgtcagagcaatggatgtgggatacatgtgcgatgatactatcacttatgaatgcccagtgctgtcggctggtaatgatccagaagacatcgactgttggtgcacaaagtcagcagtctacgtcaggtatggaagatgcaccaagacacgccactcaagacgcagtcggaggtcactgacagtgcagacacacggagaaagcactctagcgaacaagaagggggcttggatggacagcaccaaggccacaaggtatttggtaaaaacagaatcatggatcttgaggaaccctggatatgccctggtggcagccgtcattggttggatgcttgggagcaacaccatgcagagagttgtgtttgtcgtgctattgcttttggtggccccagcttacagctttaactgccttggaatgagcaacagagacttcttggaaggagtgtctggagcaacatgggtggatttggttctcgaaggcgacagctgcgtgactatcatgtctaaggacaagcctaccatcgatgtgaagatgatgaatatggaggcggccaacctggcagaggtccgcagttattgctatttggctaccgtcagcgatctctccaccaaagctgcgtgcccggccatgggagaagctcacaatgacaaacgtgctgacccagcttttgtgtgcagacaaggagtggtggacaggggctggggcaacggctgcggactatttggcaaaggaagcattgacacatgcgccaaatttgcctgctctaccaaggcaataggaagaaccattttgaaagagaatatcaagtacgaagtggccatttttgtccatggaccaactactgtggagtcgcacggaaactactccacacaggttggagccactcaggcagggagattcagcatcactcctgcggcgccttcatacacactaaagcttggagaatatggagaggtgacagtggactgtgaaccacggtcagggattgacaccaatgcatactacgtgatgactgttggaacaaagacgttcttggtccatcgtgagtggttcatggacctcaacctcccttggagcagtgctggaagtactgtgtggaggaacagagagacgttaatggagtttgaggaaccacacgccacgaagcagtctgtgatagcattgggctcacaagagggagctctgcatcaagctttggctggagccattcctgtggaattttcaagcaacactgtcaagttgacgtcgggtcatttgaagtgtagagtgaagatggaaaaattgcagttgaagggaacaacctatggcgtctgttcaaaggctttcaagtttcttgggactcccgcagacacaggtcacggcactgtggtgttggaattgcagtacactggcacggatggaccttgcaaagttcctatctcgtcagtggcttcattgaacgacctaacgccagtgggcagattggtcactgtcaacccttttgtttcagtggccacggccaacgctaaggtcctgattgaattggaaccaccctttggagactcatacatagtggtgggcagaggagaacaacagatcaatcaccactggcacaagtctggaagcagcattggcaaagcctttacaaccaccctcaaaggagcgcagagactagccgctctaggagacacagcttgggactttggatcagttggaggggtgttcacctcagttgggaaggctgtccatcaagtgttcggaggagcattccgctcactgttcggaggcatgtcctggataacgcaaggattgctgggggctctcctgttgtggatgggcatcaatgctcgtgacaggtccatagctctcacgtttctcgcagttggaggagttctgctcttcctctccgtgaacgtgcacgctgacactgggtgtgccatagacatcagccggcaagagctgagatgtggaagtggagtgttcatacacaatgatgtggaggcttggatggaccggtacaagtattaccctgaaacgccacaaggcctagccaagatcattcagaaagctcataaggaaggagtgtgcggtctacgatcagtttccagactggagcatcaaatgtgggaagcagtgaaggacgagctgaacactcttttgaaggagaatggtgtggaccttagtgtcgtggttgagaaacaagggggaatgtacaagtcagcacctaaacgcctcaccgccaccacggaaaaattggaaattggctggaaggcctggggaaagagtattttgtttgcaccagaactcgccaacaacacctttgtggttgatggtccggagaccaaggaatgtccgactcagaatcgcgcttggaatagcttagaagtggaggattttggatttggtctcaccagcactcggatgttcctgaaggtcagagagagcaacacaactgaatgtgactcgaagatcattggaacggctgtcaagaacaacttggcgatccacagtgacctgtcctattggattgaaagcaggctcaatgatacgtggaagcttgaaagggcagttctgggtgaagtcaaatcatgtacgtggcctgagacgcataccttgtggggcgatggaatccttgagagtgacttgataataccagtcacactggcgggaccacgaagcaatcacaatcggagacctgggtataagacacaaaaccagggcccatgggacgaaggccgggtagagattgacttcgattactgcccaggaactacggtcaccctgagtgagagctgcggacaccgtggacctgccactcgcaccaccacagagagcggaaagttgataacagattggtgctgcaggagctgcaccttaccaccactgcgctaccaaactgacagcggctgttggtatggtatggagatcagaccacagagacatgatgaaaagaccctcgtgcagtcacaagtgaatgcttataatgctgatatgattgacccttttcagttgggccttctggtcgtgttcttggccacccaggaggtccttcgcaagaggtggacagccaagatcagcatgccagctatactgattgctctgctagtcctggtgtttgggggcattacttacactgatgtgttacgctatgtcatcttggtgggggcagctttcgcagaatctaattcgggaggagacgtggtacacttggcgctcatggcgaccttcaagatacaaccagtgtttatggtggcatcgtttcttaaagcgagatggaccaaccaggagaacattttgttgatgttggcggctgttttctttcaaatggcttatcacgatgcccgccaaattctgctctgggagatccctgatgtgttgaattcactggcaatagcttggatgatactgagagccataacattcacaacgacatcaaacgtggttgttccgctgctagccctgctaacacccgggctgagatgcttgaatctggatgtgtacaggatactgctgttgatggtcggaataggcagcttgatcagggagaagaggagcgcagctgcaaaaaagaaaggagcaagtctgctatgcttggctctagcctcaacaggactcttcaaccccatgatccttgctgctggactgattgcatgtgatcccaaccgtaaacgcgggtggcccgcaactgaagtgatgacagctgtcggcctaatgtttgccatcgtcggagggctggcagagcttgacattgactccatggccattccaatgactatcgcggggctcatgtttgctgctttcgtgatttctgggaaatcaacagatatgtggattgagagaacggcggacatttcctgggaaagtgatgcagagattacaggctcgagcgaaagagttgatgtgcggcttgatgatgatggaaacttccagctcatgaatgatccaggagcaccttggaagatatggatgctcagaatggtctgtctcgcgattagtgcgtacaccccctgggcaatcttgccctcagtagttggattttggataactctccaatacacaaagagaggaggcgtgttgtgggacactccctcaccaaaggagtacaaaaagggggacacgaccaccggcgtctacaggatcatgactcgtgggctgctcggcagttatcaagcaggagcaggcgtgatggttgaaggtgttttccacaccctttggcatacaacaaaaggagccgctttgatgagcggagagggccgcctggacccatactggggcagtgtcaaggaggatcgactttgttacggaggaccctggaaattgcagcacaagtggaacgggcaggatgaggtgcagatgattgtggtggaacctggcaagaacgttaagaacgtccagacgaaaccaggggtgttcaaaacacctgaaggagaaatcggggccgtgactttggacttccccactggaacatcaggctcaccaatagtggacaaaaacggtgatgtgattgggctttatggcaatggagtcataatgcccaacggctcatacataagcgcgatagtgcagggtgaaaggatggatgagccaatcccagccggattcgaacctgagatgctgaggaaaaaacagatcactgtactggatctccatcccggcgccggtaaaacaaggaggattctgccacagatcatcaaagaggccataaacagaagactgagaacagccgtgctagcaccaaccagggttgtggctgctgagatggctgaagcactgagaggactgcccatccggtaccagacatccgcagtgcccagagaacataatggaaatgagattgttgatgtcatgtgtcatgctaccctcacccacaggctgatgtctcctcacagggtgccgaactacaacctgttcgtgatggatgaggctcatttcaccgacccagctagcattgcagcaagaggttacatttccacaaaggtcgagctaggggaggcggcggcaatattcatgacagccaccccaccaggcacttcagatccattcccagagtccaattcaccaatttccgacttacagactgagatcccggatcgagcttggaactctggatacgaatggatcacagaatacaccgggaagacggtttggtttgtgcctagtgttaagatggggaatgagattgccctttgcctacaacgtgctggaaagaaagtagtccaattgaacagaaagtcgtacgagacggagtacccaaaatgtaagaacgatgattgggactttgttatcacaacagacatatctgaaatgggggctaacttcaaggcgagcagggtgattgacagccggaagagtgtgaaaccaaccatcataacagaaggagaagggagagtgatcctgggagaaccatctgcagtgacagcagctagtgccgcccagagacgtggacgtatcggtagaaatccgtcgcaagttggtgatgagtactgttatggggggcacacgaatgaagacgactcgaacttcgcccattggactgaggcacgaatcatgctggacaacatcaacatgccaaacggactgatcgctcaattctaccaaccagagcgtgagaaggtatataccatggatggggaataccggctcagaggagaagagagaaaaaactttctggaactgttgaggactgcagatctgccagtttggctggcttacaaggttgcagcggctggagtgtcataccacgaccggaggtggtgctttgatggtcctaggacaaacacaattttagaagacaacaacgaagtggaagtcatcacgaagcttggtgaaaggaagattctgaggccgcgctggattgacgccagggtgtactcggatcaccaggcactaaaggcgttcaaggacttcgcctcgggaaaacgttctcagatagggctcattgaggttctgggaaagatgcctgagcacttcatggggaagacatgggaagcacttgacaccatgtacgttgtggccactgcagagaaaggaggaagagctcacagaatggccctggaggaactgccagatgctcttcagacaattgccttgattgccttattgagtgtgatgaccatgggagtattcttcctcctcatgcagcggaagggcattggaaagataggtttgggaggcgctgtcttgggagtcgcgacctttttctgttggatggctgaagttccaggaacgaagatcgccggaatgttgctgctctcccttctcttgatgattgtgctaattcctgagccagagaagcaacgttcgcagacagacaaccagctagccgtgttcctgatatgtgtcatgacccttgtgagcgcagtggcagccaacgagatgggttggctagataagaccaagagtgacataagcagtttgtttgggcaaagaattgaggtcaaggagaatttcagcatgggagagtttcttctggacttgaggccggcaacagcctggtcactgtacgctgtgacaacagcggtcctcactccactgctaaagcatttgatcacgtcagattacatcaacacctcattgacctcaataaacgttcaggcaagtgcactattcacactcgcgcgaggcttccccttcgtcgatgttggagtgtcggctctcctgctagcagccggatgctggggacaagtcaccctcaccgttacggtaacagcggcaacactccttttttgccactatgcctacatggttcccggttggcaagctgaggcaatgcgctcagcccagcggcggacagcggccggaatcatgaagaacgctgtagtggatggcatcgtggccacggacgtcccagaattagagcgcaccacacccatcatgcagaagaaaattggacagatcatgctgatcttggtgtctctagctgcagtagtagtgaacccgtctgtgaagacagtacgagaagccggaattttgatcacggccgcagcggtgacgctttgggagaatggagcaagctctgtttggaacgcaacaactgccatcggactctgccacatcatgcgtgggggttggttgtcatgtctatccataacatggacactcataaagaacatggaaaaaccaggactaaaaagaggtggggcaaaaggacgcaccttgggagaggtttggaaagaaagactcaaccagatgacaaaagaagagttcactaggtaccgcaaagaggccatcatcgaagtcgatcgctcagcggcaaaacacgccaggaaagaaggcaatgtcactggagggcatccagtctctaggggcacagcaaaactgagatggctggtcgaacggaggtttctcgaaccggtcggaaaagtgattgaccttggatgtggaagaggcggttggtgttactatatggcaacccaaaaaagagtccaagaagtcagagggtacacaaagggcggtcccggacatgaagagccccaactagtgcaaagttatggatggaacattgtcaccatgaagagtggagtggatgtgttctacagaccttctgagtgttgtgacaccctcctttgtgacatcggagagtcctcgtcaagtgctgaggttgaagagcataggacgattcgggtccttgaaatggttgaggactggctgcaccgagggccaagggaattttgcgtgaaggtgctctgcccctacatgccgaaagtcatagagaagatggagctgctccaacgccggtatggggggggactggtcagaaacccactctcacggaattccacgcacgagatgtattgggtgagtcgagcttcaggcaatgtggtacattcagtgaatatgaccagccaggtgctcctaggaagaatggaaaaaaggacctggaagggaccccaatacgaggaagacgtaaacttgggaagtggaaccagggcggtgggaaaacccctgctcaactcagacaccagtaaaatcaagaacaggattgaacgactcaggcgtgagtacagttcgacgtggcaccacgatgagaaccacccatatagaacctggaactatcatggcagttatgatgtgaagcccacaggctccgccagttcgctggtcaatggagtggtcaggctcctctcaaaaccatgggacaccatcacgaatgttaccaccatggccatgactgacactactcccttcgggcagcagcgagtgttcaaagagaaggtggacacgaaagctcctgaaccgccagaaggagtgaagtacgtgctcaacgagaccaccaactggttgtgggcgtttttggccagagaaaaacgtcccagaatgtgctctcgagaggaattcataagaaaggtcaacagcaatgcagctttgggtgccatgtttgaagagcagaatcaatggaggagcgccagagaagcagttgaagatccaaaattttgggaaatggtggatgaggagcgcgaggcacatctgcggggggaatgtcacacttgcatttacaacatgatgggaaagagagagaaaaaacccggagagttcggaaaggccaagggaagcagagccatttggttcatgtggctcggagctcgctttctggagttcgaggctctgggttttctcaatgaagaccactggcttggaagaaagaactcaggaggaggtgtcgagggcttgggcctccaaaaactgggttacatcctgcgtgaagttggcatccggcctgggggcaagatctatgctgatgacacagctggctgggacacccgcatcacgagagctgacttggaaaatgaagctaaggtgcttgagctgcttgatggggaacatcggcgtcttgccagggccatcattgagctcacctatcgtcacaaagttgtgaaagtgatgcgcccggctgctgatggaagaaccgttatggatgttatctccagagaagatcagagggggagtggacaagttgtcacctacgccctaaacactttcaccaacctggctgtccagctggtgaggatgatggaaggggaaggagtgattggcccagatgatgtggagaaactcacaaaagggaaaggacccaaagtcaggacctggctgtttgagaatggggaagaaagactcagccgcatggctgtcagtggagatgactgtgtggtaaagcccctggacgatcgctttgccacctcgctccacttcctcaatgctatgtcaaaggttcgcaaagacatccaagagtggaaaccgtcaactggatggtatgattggcagcaggttccattttgctcaaaccatttcactgaattgatcatgaaagatggaagaacactggtggttccatgccgaggacaggatgaattggtaggcagagctcgcatatctccaggggccggatggaacgtccgcgacactgcttgtctggctaagtcttatgcccagatgtggctgcttctgtacttccacagaagagacctgcggctcatggccaacgccatttgctccgctgtccctgtgaattgggtccctaccggaagaaccacgtggtccatccatgcaggaggagagtggatgacaacagaggacatgttggaggtctggaaccgtgtttggatagaggagaatgaatggatggaagacaaaaccccagtggagaaatggagtgacgtcccatattcaggaaaacgagaggacatctggtgtggcagcctgattggcacaagagcccgagccacgtgggcagaaaacatccaggtggctatcaaccaagtcagagcaatcatcggagatgagaagtatgtggattacatgagttcactaaagagatatgaagacacaactttggttgaggacacagtactgtagatatttaatcaattgtaaatagacaatataagtatgcataaaagtgtagttttatagtagtatttagtggtgttagtgtaaatagttaagaaaatcttgaggagaaagtcaggccgggaagttcccgccaccggaagttgagtagacggtgctgcctgcgactcaaccccaggaggactgggtgaacaaagccgcgaagtgatccatgtaagccctcagaaccgtctcggaaggaggaccccacatgttgtaacttcaaagcccaatgtcagaccacgctacggcgtgctactctgcggagagtgcagtctgcgatagtgccccaggaggactgggttaacaaaggcaaaccaacgccccacgcggcccaagccccggtaatggtgttaaccagggcgaaaggactagaggttagaggagaccccgcggtttaaagtgcacggcccagcctggctgaagctgtaggtcaggggaaggactagaggttagtggagaccccgtgccacaaaacaccacaacaaaacagcatattgacacctgggatagactaggagatcttctgctctgcacaaccagccacacggcacagtgcgccgacaatggtggctggtggtgcgagaacacaggatct

SEQ ID NO: 87 - DNA, 12475 bp, Artificial Sequence
  Synthetic Construct
tcatcaagcggacacactcgactgtttgaatcatcacaaacactcctaattgttgttaattgtgtcacgctcgacaaagaatcgtgcttctagagctacagattctttggtcctcccgggccgttctcggcccgacagttatacgattttgcgccttacggggcgcacaacaggaactaacctgaattcgttttcttcgctcccccgttctgaccatatcgacactagtaccttgacgggtagtagttccggttgcggtagtggtggtaggactagcggcactggaagacgaagcggtcgtcggtcttgtagtggctccttaagatggtctcgtggacgtcgcggcactcgttcccgatggactcgcgggacgcctggccgaccatgtggtcgcactagtggtagctcgacaggttgtagtttcttttgttcacgttgccgtggctgcggttccactttgactagttcgtccttgacctgttcatgttcttgcggcactggctcgacgtcgacgactacgtctcgtggggacggcggttgttggcccggtctgcgctcgacggggccaagtacttgatgtgggacttgttgcggttcttttggttgcactgggactcgttcttcgccttcgccgccaaggacccgaaggacgacccgcacccgtcgcggtagcggtcgccgtagcggcacaggttccacgacgtggaccttccgctccacttgttctagttcaggcgggacgacaggtggttgttccggcaccacagggactcgttgccgcactcgcacgactggtcgttccacgacctagacttcttgatgtagctgttcgtcgacgacgggtagcacttgttcgtctcgacgtcgtagtcgttgtagctctggcactagctcaaggtcgtcttcttgttggccgacgacctttagtgggccctcaagtcgcacttgcggccgcactggtgggggcactcgtggatgtacgactggttgtcgctcgacgacagggactagttactgtacgggtagtggttgctggtcttctttgactactcgttgttgcacgtctagcacgccgtcgtctcgatgaggtagtactcgtagtagtttcttctccacgaccggatgcaccacgtcgacggggacatgccgcactagctgtgggggacgaccttcgacgtgtggtcgggggacacgtggtggttgtggtttctcccgtcgttgtagacggactgggcctggctggccccgaccatgacgttgttgcggccgtcgcactcgaagaagggggaccggctgtggacgttccacgtctcgttggcccacaagacgctgtggtacttgtcggactgggacgggaggctccacttggacacgttgtagctgtagaagttggggttcatgctgacgttctagtactggaggttctggctgcactcgtcgaggcactagtggagggacccgcggtagcactcgacgatgccgttctggttcacgtggcggtcgttgttcttggccccgtagtagttctggaagtcgttgccgacgctgatgcactcgttgttcccgcacctgtggcactcgcacccgttgtgtgacatgatgcacttattcgtccttccgttctcggacatgcacttcccgctcggatagtagttgaagatgctgggggaccacaaggggtcgctgctcaagctgcggtcgtagtcggtccacttgctcttctagttggtctcggaccggaagtaggccttctcgctgctcgacgacgtgttacacttacggccgttctcgtggtggttatagtacttaaaactagacgagtttgaacgtccgctacatcttagtttaggacctgggcctcctttctggccataacgtcagtactaaccggactagcggacgcatcctcgtcaatgggagagattgaaggttcccttccactactactgccatttacgatgactgcagtgtctacagtagtgctaaggttgtcgacgacctttcttggatacgtaacagtctcgttacctacaccctatgtacacgctactatgatagtgaatacttacgggtcacgacagccgaccattactaggtcttctgtagctgacaaccacgtgtttcagtcgtcagatgcagtccataccttctacgtggttctgtgcggtgagttctgcgtcagcctccagtgactgtcacgtctgtgtgcctctttcgtgagatcgcttgttcttcccccgaacctacctgtcgtggttccggtgttccataaaccatttttgtcttagtacctagaactccttgggacctatacgggaccaccgtcggcagtaaccaacctacgaaccctcgttgtggtacgtctctcaacacaaacagcacgataacgaaaaccaccggggtcgaatgtcgaaattgacggaaccttactcgttgtctctgaagaaccttcctcacagacctcgttgtacccacctaaaccaagagcttccgctgtcgacgcactgatagtacagattcctgttcggatggtagctacacttctactacttatacctccgccggttggaccgtctccaggcgtcaataacgataaaccgatggcagtcgctagagaggtggtttcgacgcacgggccggtaccctcttcgagtgttactgtttgcacgactgggtcgaaaacacacgtctgttcctcaccacctgtccccgaccccgttgccgacgcctgataaaccgtttccttcgtaactgtgtacgcggtttaaacggacgagatggttccgttatccttcttggtaaaactttctcttatagttcatgcttcaccggtaaaaacaggtacctggttgatgacacctcagcgtgcctttgatgaggtgtgtccaacctcggtgagtccgtccctctaagtcgtagtgaggacgccgcggaagtatgtgtgatttcgaacctcttatacctctccactgtcacctgacacttggtgccagtccctaactgtggttacgtatgatgcactactgacaaccttgtttctgcaagaaccaggtagcactcaccaagtacctggagttggagggaacctcgtcacgaccttcatgacacacctccttgtctctctgcaattacctcaaactccttggtgtgcggtgcttcgtcagacactatcgtaacccgagtgttctccctcgagacgtagttcgaaaccgacctcggtaaggacaccttaaaagttcgttgtgacagttcaactgcagcccagtaaacttcacatctcacttctacctttttaacgtcaacttcccttgttggataccgcagacaagtttccgaaagttcaaagaaccctgagggcgtctgtgtccagtgccgtgacaccacaaccttaacgtcatgtgaccgtgcctacctggaacgtttcaaggatagagcagtcaccgaagtaacttgctggattgcggtcacccgtctaaccagtgacagttgggaaaacaaagtcaccggtgccggttgcgattccaggactaacttaaccttggtgggaaacctctgagtatgtatcaccacccgtctcctcttgttgtctagttagtggtgaccgtgttcagaccttcgtcgtaaccgtttcggaaatgttggtgggagtttcctcgcgtctctgatcggcgagatcctctgtgtcgaaccctgaaacctagtcaacctccccacaagtggagtcaacccttccgacaggtagttcacaagcctcctcgtaaggcgagtgacaagcctccgtacaggacctattgcgttcctaacgacccccgagaggacaacacctacccgtagttacgagcactgtccaggtatcgagagtgcaaagagcgtcaacctcctcaagacgagaaggagaggcacttgcacgtgcgactgtgacccacacggtatctgtagtcggccgttctcgactctacaccttcacctcacaagtatgtgttactacacctccgaacctacctggccatgttcataatgggactttgcggtgttccggatcggttctagtaagtctttcgagtattccttcctcacacgccagatgctagtcaaaggtctgacctcgtagtttacacccttcgtcacttcctgctcgacttgtgagaaaacttcctcttaccacacctggaatcacagcaccaactctttgttcccccttacatgttcagtcgtggatttgcggagtggcggtggtgcctttttaacctttaaccgaccttccggacccctttctcataaaacaaacgtggtcttgagcggttgttgtggaaacaccaactaccaggcctctggttccttacaggctgagtcttagcgcgaaccttatcgaatcttcacctcctaaaacctaaaccagagtggtcgtgagcctacaaggacttccagtctctctcgttgtgttgacttacactgagcttctagtaaccttgccgacagttcttgttgaaccgctaggtgtcactggacaggataacctaactttcgtccgagttactatgcaccttcgaactttcccgtcaagacccacttcagtttagtacatgcaccggactctgcgtatggaacaccccgctaccttaggaactctcactgaactattatggtcagtgtgaccgccctggtgcttcgttagtgttagcctctggacccatattctgtgttttggtcccgggtaccctgcttccggcccatctctaactgaagctaatgacgggtccttgatgccagtgggactcactctcgacgcctgtggcacctggacggtgagcgtggtggtgtctctcgcctttcaactattgtctaaccacgacgtcctcgacgtggaatggtggtgacgcgatggtttgactgtcgccgacaaccataccatacctctagtctggtgtctctgtactacttttctgggagcacgtcagtgttcacttacgaatattacgactatactaactgggaaaagtcaacccggaagaccagcacaagaaccggtgggtcctccaggaagcgttctccacctgtcggttctagtcgtacggtcgatatgactaacgagacgatcaggaccacaaacccccgtaatgaatgtgactacacaatgcgatacagtagaaccacccccgtcgaaagcgtcttagattaagccctcctctgcaccatgtgaaccgcgagtaccgctggaagttctatgttggtcacaaataccaccgtagcaaagaatttcgctctacctggttggtcctcttgtaaaacaactacaaccgccgacaaaagaaagtttaccgaatagtgctacgggcggtttaagacgagaccctctagggactacacaacttaagtgaccgttatcgaacctactatgactctcggtattgtaagtgttgctgtagtttgcaccaacaaggcgacgatcgggacgattgtgggcccgactctacgaacttagacctacacatgtcctatgacgacaactaccagccttatccgtcgaactagtccctcttctcctcgcgtcgacgttttttctttcctcgttcagacgatacgaaccgagatcggagttgtcctgagaagttggggtactaggaacgacgacctgactaacgtacactagggttggcatttgcgcccaccgggcgttgacttcactactgtcgacagccggattacaaacggtagcagcctcccgaccgtctcgaactgtaactgaggtaccggtaaggttactgatagcgccccgagtacaaacgacgaaagcactaaagaccctttagttgtctatacacctaactctcttgccgcctgtaaaggaccctttcactacgtctctaatgtccgagctcgctttctcaactacacgccgaactactactacctttgaaggtcgagtacttactaggtcctcgtggaaccttctatacctacgagtcttaccagacagagcgctaatcacgcatgtgggggacccgttagaacgggagtcatcaacctaaaacctattgagaggttatgtgtttctctcctccgcacaacaccctgtgagggagtggtttcctcatgtttttccccctgtgctggtggccgcagatgtcctagtactgagcacccgacgagccgtcaatagttcgtcctcgtccgcactaccaacttccacaaaaggtgtgggaaaccgtatgttgttttcctcggcgaaactactcgcctctcccggcggacctgggtatgaccccgtcacagttcctcctagctgaaacaatgcctcctgggacctttaacgtcgtgttcaccttgcccgtcctactccacgtctactaacaccaccttggaccgttcttgcaattcttgcaggtctgctttggtccccacaagttttgtggacttcctctttagccccggcactgaaacctgaaggggtgaccttgtagtccgagtggttatcacctgtttttgccactacactaacccgaaataccgttacctcagtattacgggttgccgagtatgtattcgcgctatcacgtcccactttcctacctactcggttagggtcggcctaagcttggactctacgactccttttttgtctagtgacatgacctagaggtagggccgcggccattttgttcctcctaagacggtgtctagtagtttctccggtatttgtcttctgactcttgtcggcacgatcgtggttggtcccaacaccgacgactctaccgacttcgtgactctcctgacgggtaggccatggtctgtaggcgtcacgggtctcttgtattacctttactctaacaactacagtacacagtacgatgggagtgggtgtccgactacagaggagtgtcccacggcttgatgttggacaagcactacctactccgagtaaagtggctgggtcgatcgtaacgtcgttctccaatgtaaaggtgtttccagctcgatcccctccgccgccgttataagtactgtcggtggggtggtccgtgaagtctaggtaagggtctcaggttaagtggttaaaggctgaatgtctgactctagggcctagctcgaaccttgagacctatgcttacctagtgtcttatgtggcccttctgccaaaccaaacacggatcacaattctaccccttactctaacgggaaacggatgttgcacgacctttctttcatcaggttaacttgtctttcagcatgctctgcctcatgggttttacattcttgctactaaccctgaaacaatagtgttgtctgtatagactttacccccgattgaagttccgctcgtcccactaactgtcggccttctcacactttggttggtagtattgtcttcctcttccctctcactaggaccctcttggtagacgtcactgtcgtcgatcacggcgggtctctgcacctgcatagccatctttaggcagcgttcaaccactactcatgacaataccccccgtgtgcttacttctgctgagcttgaagcgggtaacctgactccgtgcttagtacgacctgttgtagttgtacggtttgcctgactagcgagttaagatggttggtctcgcactcttccatatatggtacctaccccttatggccgagtctcctcttctctcttttttgaaagaccttgacaactcctgacgtctagacggtcaaaccgaccgaatgttccaacgtcgccgacctcacagtatggtgctggcctccaccacgaaactaccaggatcctgtttgtgttaaaatcttctgttgttgcttcaccttcagtagtgcttcgaaccactttccttctaagactccggcgcgacctaactgcggtcccacatgagcctagtggtccgtgatttccgcaagttcctgaagcggagcccttttgcaagagtctatcccgagtaactccaagaccctttctacggactcgtgaagtaccccttctgtacccttcgtgaactgtggtacatgcaacaccggtgacgtctctttcctccttctcgagtgtcttaccgggacctccttgacggtctacgagaagtctgttaacggaactaacggaataactcacactactggtaccctcataagaaggaggagtacgtcgccttcccgtaacctttctatccaaaccctccgcgacagaaccctcagcgctggaaaaagacaacctaccgacttcaaggtccttgcttctagcggccttacaacgacgagagggaagagaactactaacacgattaaggactcggtctcttcgttgcaagcgtctgtctgttggtcgatcggcacaaggactatacacagtactgggaacactcgcgtcaccgtcggttgctctacccaaccgatctattctggttctcactgtattcgtcaaacaaacccgtttcttaactccagttcctcttaaagtcgtaccctctcaaagaagacctgaactccggccgttgtcggaccagtgacatgcgacactgttgtcgccaggagtgaggtgacgatttcgtaaactagtgcagtctaatgtagttgtggagtaactggagttatttgcaagtccgttcacgtgataagtgtgagcgcgctccgaaggggaagcagctacaacctcacagccgagaggacgatcgtcggcctacgacccctgttcagtgggagtggcaatgccattgtcgccgttgtgaggaaaaaacggtgatacggatgtaccaagggccaaccgttcgactccgttacgcgagtcgggtcgccgcctgtcgccggccttagtacttcttgcgacatcacctaccgtagcaccggtgcctgcagggtcttaatctcgcgtggtgtgggtagtacgtcttcttttaacctgtctagtacgactagaaccacagagatcgacgtcatcatcacttgggcagacacttctgtcatgctcttcggccttaaaactagtgccggcgtcgccactgcgaaaccctcttacctcgttcgagacaaaccttgcgttgttgacggtagcctgagacggtgtagtacgcacccccaaccaacagtacagataggtattgtacctgtgagtatttcttgtacctttttggtcctgatttttctccaccccgttttcctgcgtggaaccctctccaaacctttctttctgagttggtctactgttttcttctcaagtgatccatggcgtttctccggtagtagcttcagctagcgagtcgccgttttgtgcggtcctttcttccgttacagtgacctcccgtaggtcagagatccccgtgtcgttttgactctaccgaccagcttgcctccaaagagcttggccagccttttcactaactggaacctacaccttctccgccaaccacaatgatataccgttgggttttttctcaggttcttcagtctcccatgtgtttcccgccagggcctgtacttctcggggttgatcacgtttcaatacctaccttgtaacagtggtacttctcacctcacctacacaagatgtctggaagactcacaacactgtgggaggaaacactgtagcctctcaggagcagttcacgactccaacttctcgtatcctgctaagcccaggaactttaccaactcctgaccgacgtggctcccggttcccttaaaacgcacttccacgagacggggatgtacggctttcagtatctcttctacctcgacgaggttgcggccatacccccccctgaccagtctttgggtgagagtgccttaaggtgcgtgctctacataacccactcagctcgaagtccgttacaccatgtaagtcacttatactggtcggtccacgaggatccttcttaccttttttcctggaccttccctggggttatgctccttctgcatttgaacccttcaccttggtcccgccacccttttggggacgagttgagtctgtggtcattttagttcttgtcctaacttgctgagtccgcactcatgtcaagctgcaccgtggtgctactcttggtgggtatatcttggaccttgatagtaccgtcaatactacacttcgggtgtccgaggcggtcaagcgaccagttacctcaccagtccgaggagagttttggtaccctgtggtagtgcttacaatggtggtaccggtactgactgtgatgagggaagcccgtcgtcgctcacaagtttctcttccacctgtgctttcgaggacttggcggtcttcctcacttcatgcacgagttgctctggtggttgaccaacacccgcaaaaaccggtctctttttgcagggtcttacacgagagctctccttaagtattctttccagttgtcgttacgtcgaaacccacggtacaaacttctcgtcttagttacctcctcgcggtctcttcgtcaacttctaggttttaaaaccctttaccacctactcctcgcgctccgtgtagacgccccccttacagtgtgaacgtaaatgttgtactaccctttctctctcttttttgggcctctcaagcctttccggttcccttcgtctcggtaaaccaagtacaccgagcctcgagcgaaagacctcaagctccgagacccaaaagagttacttctggtgaccgaaccttctttcttgagtcctcctccacagctcccgaacccggaggtttttgacccaatgtaggacgcacttcaaccgtaggccggacccccgttctagatacgactactgtgtcgaccgaccctgtgggcgtagtgctctcgactgaaccttttacttcgattccacgaactcgacgaactaccccttgtagccgcagaacggtcccggtagtaactcgagtggatagcagtgtttcaacactttcactacgcgggccgacgactaccttcttggcaatacctacaatagaggtctcttctagtctccccctcacctgttcaacagtggatgcgggatttgtgaaagtggttggaccgacaggtcgaccactcctactaccttccccttcctcactaaccgggtctactacacctctttgagtgttttccctttcctgggtttcagtcctggaccgacaaactcttaccccttctttctgagtcggcgtaccgacagtcacctctactgacacaccatttcggggacctgctagcgaaacggtggagcgaggtgaaggagttacgatacagtttccaagcgtttctgtaggttctcacctttggcagttgacctaccatactaaccgtcgtccaaggtaaaacgagtttggtaaagtgacttaactagtactttctaccttcttgtgaccaccaaggtacggctcctgtcctacttaaccatccgtctcgagcgtatagaggtccccggcctaccttgcaggcgctgtgacgaacagaccgattcagaatacgggtctacaccgacgaagacatgaaggtgtcttctctggacgccgagtaccggttgcggtaaacgaggcgacagggacacttaacccagggatggccttcttggtgcaccaggtaggtacgtcctcctctcacctactgttgtctcctgtacaacctccagaccttggcacaaacctatctcctcttacttacctaccttctgttttggggtcacctctttacctcactgcagggtataagtccttttgctctcctgtagaccacaccgtcggactaaccgtgttctcgggctcggtgcacccgtcttttgtaggtccaccgatagttggttcagtctcgttagtagcctctactcttcatacacctaatgtactcaagtgatttctctatacttctgtgttgaaaccaactcctgtgtcatgacatctataaattagttaacatttatctgttatattcatacgtattttcacatcaaaatatcatcataaatcaccacaatcacatttatcaattcttttagaactcctctttcagtccggcccttcaagggcggtggccttcaactcatctgccacgacggacgctgagttggggtcctcctgacccacttgtttcggcgcttcactaggtacattcgggagtcttggcagagccttcctcctggggtgtacaacattgaagtttcgggttacagtctggtgcgatgccgcacgatgagacgcctctcacgtcagacgctatcacggggtcctcctgacccaattgtttccgtttggttgcggggtgcgccgggttcggggccattaccacaattggtcccgctttcctgatctccaatctcctctggggcgccaaatttcacgtgccgggtcggaccgacttcgacatccagtccccttcctgatctccaatcacctctggggcacggtgttttgtggtgttgttttgtcgtataactgtggaccctatctgatcctctagaagacgagacgtgttggtcggtgtgccgtgtcacgcggctgttaccaccgaccaccacgctcttgtgtcctaga

SEQ ID NO: 88 - protein, 3255 aa, Artificial Sequence
  Synthetic Construct
MSKKPGGPGKSRAVYLLKRGMPRVLSLIGLKQKKRGGKTGIAVIMELPIIKANAITTILIAVTFCFASSQNITEEFYQSTCSAVSKGYLSALRTGWYTSVITIELSNIKENKCNGTDAKVKLIKQELDKYKNAVTELQLLMQSTPAANNRARRELPRFMNYTLNNAKKTNVTLSKKRKRRFLGFLLGVGSAIASGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCNNAGSVSFFPLADTCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMNFDLLKLAGDVESNPGPARDRSIALTFLAVGGVLLFLSVNVHADTGCAIDISRQELRCGSGVFIHNDVEAWMDRYKYYPETPQGLAKIIQKAHKEGVCGLRSVSRLEHQMWEAVKDELNTLLKENGVDLSVVVEKQGGMYKSAPKRLTATTEKLEIGWKAWGKSILFAPELANNTFVVDGPETKECPTQNRAWNSLEVEDFGFGLTSTRMFLKVRESNTTECDSKIIGTAVKNNLAIHSDLSYWIESRLNDTWKLERAVLGEVKSCTWPETHTLWGDGILESDLIIPVTLAGPRSNHNRRPGYKTQNQGPWDEGRVEIDFDYCPGTTVTLSESCGHRGPATRTTTESGKLITDWCCRSCTLPPLRYQTDSGCWYGMEIRPQRHDEKTLVQSQVNAYNADMIDPFQLGLLVVFLATQEVLRKRWTAKISMPAILIALLVLVFGGITYTDVLRYVILVGAAFAESNSGGDVVHLALMATFKIQPVFMVASFLKARWTNQENILLMLAAVFFQMAYHDARQILLWEIPDVLNSLAIAWMILRAITFTTTSNVVVPLLALLTPGLRCLNLDVYRILLLMVGIGSLIREKRSAAAKKKGASLLCLALASTGLFNPMILAAGLIACDPNRKRGWPATEVMTAVGLMFAIVGGLAELDIDSMAIPMTIAGLMFAAFVISGKSTDMWIERTADISWESDAEITGSSERVDVRLDDDGNFQLMNDPGAPWKIWMLRMVCLAISAYTPWAILPSVVGFWITLQYTKRGGVLWDTPSPKEYKKGDTTTGVYRIMTRGLLGSYQAGAGVMVEGVFHTLWHTTKGAALMSGEGRLDPYWGSVKEDRLCYGGPWKLQHKWNGQDEVQMIVVEPGKNVKNVQTKPGVFKTPEGEIGAVTLDFPTGTSGSPIVDKNGDVIGLYGNGVIMPNGSYISAIVQGERMDEPIPAGFEPEMLRKKQITVLDLHPGAGKTRRILPQIIKEAINRRLRTAVLAPTRVVAAEMAEALRGLPIRYQTSAVPREHNGNEIVDVMCHATLTHRLMSPHRVPNYNLFVMDEAHFTDPASIAARGYISTKVELGEAAAIFMTATPPGTSDPFPESNSPISDLQTEIPDRAWNSGYEWITEYTGKTVWFVPSVKMGNEIALCLQRAGKKVVQLNRKSYETEYPKCKNDDWDFVITTDISEMGANFKASRVIDSRKSVKPTIITEGEGRVILGEPSAVTAASAAQRRGRIGRNPSQVGDEYCYGGHTNEDDSNFAHWTEARIMLDNINMPNGLIAQFYQPEREKVYTMDGEYRLRGEERKNFLELLRTADLPVWLAYKVAAAGVSYHDRRWCFDGPRTNTILEDNNEVEVITKLGERKILRPRWIDARVYSDHQALKAFKDFASGKRSQIGLIEVLGKMPEHFMGKTWEALDTMYVVATAEKGGRAHRMALEELPDALQTIALIALLSVMTMGVFFLLMQRKGIGKIGLGGAVLGVATFFCWMAEVPGTKIAGMLLLSLLLMIVLIPEPEKQRSQTDNQLAVFLICVMTLVSAVAANEMGWLDKTKSDISSLFGQRIEVKENFSMGEFLLDLRPATAWSLYAVTTAVLTPLLKHLITSDYINTSLTSINVQASALFTLARGFPFVDVGVSALLLAAGCWGQVTLTVTVTAATLLFCHYAYMVPGWQAEAMRSAQRRTAAGIMKNAVVDGIVATDVPELERTTPIMQKKIGQIMLILVSLAAVVVNPSVKTVREAGILITAAAVTLWENGASSVWNATTAIGLCHIMRGGWLSCLSITWTLIKNMEKPGLKRGGAKGRTLGEVWKERLNQMTKEEFTRYRKEAIIEVDRSAAKHARKEGNVTGGHPVSRGTAKLRWLVERRFLEPVGKVIDLGCGRGGWCYYMATQKRVQEVRGYTKGGPGHEEPQLVQSYGWNIVTMKSGVDVFYRPSECCDTLLCDIGESSSSAEVEEHRTIRVLEMVEDWLHRGPREFCVKVLCPYMPKVIEKMELLQRRYGGGLVRNPLSRNSTHEMYWVSRASGNVVHSVNMTSQVLLGRMEKRTWKGPQYEEDVNLGSGTRAVGKPLLNSDTSKIKNRIERLRREYSSTWHHDENHPYRTWNYHGSYDVKPTGSASSLVNGVVRLLSKPWDTITNVTTMAMTDTTPFGQQRVFKEKVDTKAPEPPEGVKYVLNETTNWLWAFLAREKRPRMCSREEFIRKVNSNAALGAMFEEQNQWRSAREAVEDPKFWEMVDEEREAHLRGECHTCIYNMMGKREKKPGEFGKAKGSRAIWFMWLGARFLEFEALGFLNEDHWLGRKNSGGGVEGLGLQKLGYILREVGIRPGGKIYADDTAGWDTRITRADLENEAKVLELLDGEHRRLARAIIELTYRHKVVKVMRPAADGRTVMDVISREDQRGSGQVVTYALNTFTNLAVQLVRMMEGEGVIGPDDVEKLTKGKGPKVRTWLFENGEERLSRMAVSGDDCVVKPLDDRFATSLHFLNAMSKVRKDIQEWKPSTGWYDWQQVPFCSNHFTELIMKDGRTLVVPCRGQDELVGRARISPGAGWNVRDTACLAKSYAQMWLLLYFHRRDLRLMANAICSAVPVNWVPTGRTTWSIHAGGEWMTTEDMLEVWNRVWIEENEWMEDKTPVEKWSDVPYSGKREDIWCGSLIGTRARATWAENIQVAINQVRAIIGDEKYVDYMSSLKRYEDTTLVEDTVL

SEQ ID NO: 89 - DNA, 10495 bp, Artificial Sequence
  Synthetic Construct
agtagttcgcctgtgtgagctgacaaacttagtagtgtttgtgaggattaacaacaattaacacagtgcgagctgtttcttagcacgaagatctcgatgtctaagaaaccaggagggcccggcaagagccgggctgtctatttgctaaaacgcggaatgccccgcgtgttgtccttgattggacttaagcaaaagaagcgagggggcaagactggtatagctgtgatcatggaactgcccatcatcaaggccaacgccatcaccaccatcctgatcgccgtgaccttctgcttcgccagcagccagaacatcaccgaggaattctaccagagcacctgcagcgccgtgagcaagggctacctgagcgccctgcggaccggctggtacaccagcgtgatcaccatcgagctgtccaacatcaaagaaaacaagtgcaacggcaccgacgccaaggtgaaactgatcaagcaggaactggacaagtacaagaacgccgtgaccgagctgcagctgctgatgcagagcacccctgccgccaacaaccgggccagacgcgagctgccccggttcatgaactacaccctgaacaacgccaagaaaaccaacgtgaccctgagcaagaagcggaagcggcggttcctgggcttcctgctgggcgtgggcagcgccatcgccagcggcatcgccgtgtccaaggtgctgcacctggaaggcgaggtgaacaagatcaagtccgccctgctgtccaccaacaaggccgtggtgtccctgagcaacggcgtgagcgtgctgaccagcaaggtgctggatctgaagaactacatcgacaagcagctgctgcccatcgtgaacaagcagagctgcagcatcagcaacatcgagaccgtgatcgagttccagcagaagaacaaccggctgctggaaatcacccgggagttcagcgtgaacgccggcgtgaccacccccgtgagcacctacatgctgaccaacagcgagctgctgtccctgatcaatgacatgcccatcaccaacgaccagaagaaactgatgagcaacaacgtgcagatcgtgcggcagcagagctactccatcatgagcatcatcaaagaagaggtgctggcctacgtggtgcagctgcccctgtacggcgtgatcgacaccccctgctggaagctgcacaccagccccctgtgcaccaccaacaccaaagagggcagcaacatctgcctgacccggaccgaccggggctggtactgcaacaacgccggcagcgtgagcttcttccccctggccgacacctgcaaggtgcagagcaaccgggtgttctgcgacaccatgaacagcctgaccctgccctccgaggtgaacctgtgcaacatcgacatcttcaaccccaagtacgactgcaagatcatgacctccaagaccgacgtgagcagctccgtgatcacctccctgggcgccatcgtgagctgctacggcaagaccaagtgcaccgccagcaacaagaaccggggcatcatcaagaccttcagcaacggctgcgactacgtgagcaacaagggcgtggacaccgtgagcgtgggcaacacactgtactacgtgaataagcaggaaggcaagagcctgtacgtgaagggcgagcctatcatcaacttctacgaccccctggtgttccccagcgacgagttcgacgccagcatcagccaggtgaacgagaagatcaaccagagcctggccttcatccggaagagcgacgagctgctgcacaatgtgaatgccggcaagagcaccaccaatatcatgaattttgatctgctcaaacttgcaggcgatgtagaatcaaatcctggacccgcccgggacaggtccatagctctcacgtttctcgcagttggaggagttctgctcttcctctccgtgaacgtgcacgctgacactgggtgtgccatagacatcagccggcaagagctgagatgtggaagtggagtgttcatacacaatgatgtggaggcttggatggaccggtacaagtattaccctgaaacgccacaaggcctagccaagatcattcagaaagctcataaggaaggagtgtgcggtctacgatcagtttccagactggagcatcaaatgtgggaagcagtgaaggacgagctgaacactcttttgaaggagaatggtgtggaccttagtgtcgtggttgagaaacaagggggaatgtacaagtcagcacctaaacgcctcaccgccaccacggaaaaattggaaattggctggaaggcctggggaaagagtattttgtttgcaccagaactcgccaacaacacctttgtggttgatggtccggagaccaaggaatgtccgactcagaatcgcgcttggaatagcttagaagtggaggattttggatttggtctcaccagcactcggatgttcctgaaggtcagagagagcaacacaactgaatgtgactcgaagatcattggaacggctgtcaagaacaacttggcgatccacagtgacctgtcctattggattgaaagcaggctcaatgatacgtggaagcttgaaagggcagttctgggtgaagtcaaatcatgtacgtggcctgagacgcataccttgtggggcgatggaatccttgagagtgacttgataataccagtcacactggcgggaccacgaagcaatcacaatcggagacctgggtataagacacaaaaccagggcccatgggacgaaggccgggtagagattgacttcgattactgcccaggaactacggtcaccctgagtgagagctgcggacaccgtggacctgccactcgcaccaccacagagagcggaaagttgataacagattggtgctgcaggagctgcaccttaccaccactgcgctaccaaactgacagcggctgttggtatggtatggagatcagaccacagagacatgatgaaaagaccctcgtgcagtcacaagtgaatgcttataatgctgatatgattgacccttttcagttgggccttctggtcgtgttcttggccacccaggaggtccttcgcaagaggtggacagccaagatcagcatgccagctatactgattgctctgctagtcctggtgtttgggggcattacttacactgatgtgttacgctatgtcatcttggtgggggcagctttcgcagaatctaattcgggaggagacgtggtacacttggcgctcatggcgaccttcaagatacaaccagtgtttatggtggcatcgtttcttaaagcgagatggaccaaccaggagaacattttgttgatgttggcggctgttttctttcaaatggcttatcacgatgcccgccaaattctgctctgggagatccctgatgtgttgaattcactggcaatagcttggatgatactgagagccataacattcacaacgacatcaaacgtggttgttccgctgctagccctgctaacacccgggctgagatgcttgaatctggatgtgtacaggatactgctgttgatggtcggaataggcagcttgatcagggagaagaggagcgcagctgcaaaaaagaaaggagcaagtctgctatgcttggctctagcctcaacaggactcttcaaccccatgatccttgctgctggactgattgcatgtgatcccaaccgtaaacgcgggtggcccgcaactgaagtgatgacagctgtcggcctaatgtttgccatcgtcggagggctggcagagcttgacattgactccatggccattccaatgactatcgcggggctcatgtttgctgctttcgtgatttctgggaaatcaacagatatgtggattgagagaacggcggacatttcctgggaaagtgatgcagagattacaggctcgagcgaaagagttgatgtgcggcttgatgatgatggaaacttccagctcatgaatgatccaggagcaccttggaagatatggatgctcagaatggtctgtctcgcgattagtgcgtacaccccctgggcaatcttgccctcagtagttggattttggataactctccaatacacaaagagaggaggcgtgttgtgggacactccctcaccaaaggagtacaaaaagggggacacgaccaccggcgtctacaggatcatgactcgtgggctgctcggcagttatcaagcaggagcaggcgtgatggttgaaggtgttttccacaccctttggcatacaacaaaaggagccgctttgatgagcggagagggccgcctggacccatactggggcagtgtcaaggaggatcgactttgttacggaggaccctggaaattgcagcacaagtggaacgggcaggatgaggtgcagatgattgtggtggaacctggcaagaacgttaagaacgtccagacgaaaccaggggtgttcaaaacacctgaaggagaaatcggggccgtgactttggacttccccactggaacatcaggctcaccaatagtggacaaaaacggtgatgtgattgggctttatggcaatggagtcataatgcccaacggctcatacataagcgcgatagtgcagggtgaaaggatggatgagccaatcccagccggattcgaacctgagatgctgaggaaaaaacagatcactgtactggatctccatcccggcgccggtaaaacaaggaggattctgccacagatcatcaaagaggccataaacagaagactgagaacagccgtgctagcaccaaccagggttgtggctgctgagatggctgaagcactgagaggactgcccatccggtaccagacatccgcagtgcccagagaacataatggaaatgagattgttgatgtcatgtgtcatgctaccctcacccacaggctgatgtctcctcacagggtgccgaactacaacctgttcgtgatggatgaggctcatttcaccgacccagctagcattgcagcaagaggttacatttccacaaaggtcgagctaggggaggcggcggcaatattcatgacagccaccccaccaggcacttcagatccattcccagagtccaattcaccaatttccgacttacagactgagatcccggatcgagcttggaactctggatacgaatggatcacagaatacaccgggaagacggtttggtttgtgcctagtgttaagatggggaatgagattgccctttgcctacaacgtgctggaaagaaagtagtccaattgaacagaaagtcgtacgagacggagtacccaaaatgtaagaacgatgattgggactttgttatcacaacagacatatctgaaatgggggctaacttcaaggcgagcagggtgattgacagccggaagagtgtgaaaccaaccatcataacagaaggagaagggagagtgatcctgggagaaccatctgcagtgacagcagctagtgccgcccagagacgtggacgtatcggtagaaatccgtcgcaagttggtgatgagtactgttatggggggcacacgaatgaagacgactcgaacttcgcccattggactgaggcacgaatcatgctggacaacatcaacatgccaaacggactgatcgctcaattctaccaaccagagcgtgagaaggtatataccatggatggggaataccggctcagaggagaagagagaaaaaactttctggaactgttgaggactgcagatctgccagtttggctggcttacaaggttgcagcggctggagtgtcataccacgaccggaggtggtgctttgatggtcctaggacaaacacaattttagaagacaacaacgaagtggaagtcatcacgaagcttggtgaaaggaagattctgaggccgcgctggattgacgccagggtgtactcggatcaccaggcactaaaggcgttcaaggacttcgcctcgggaaaacgttctcagatagggctcattgaggttctgggaaagatgcctgagcacttcatggggaagacatgggaagcacttgacaccatgtacgttgtggccactgcagagaaaggaggaagagctcacagaatggccctggaggaactgccagatgctcttcagacaattgccttgattgccttattgagtgtgatgaccatgggagtattcttcctcctcatgcagcggaagggcattggaaagataggtttgggaggcgctgtcttgggagtcgcgacctttttctgttggatggctgaagttccaggaacgaagatcgccggaatgttgctgctctcccttctcttgatgattgtgctaattcctgagccagagaagcaacgttcgcagacagacaaccagctagccgtgttcctgatatgtgtcatgacccttgtgagcgcagtggcagccaacgagatgggttggctagataagaccaagagtgacataagcagtttgtttgggcaaagaattgaggtcaaggagaatttcagcatgggagagtttcttctggacttgaggccggcaacagcctggtcactgtacgctgtgacaacagcggtcctcactccactgctaaagcatttgatcacgtcagattacatcaacacctcattgacctcaataaacgttcaggcaagtgcactattcacactcgcgcgaggcttccccttcgtcgatgttggagtgtcggctctcctgctagcagccggatgctggggacaagtcaccctcaccgttacggtaacagcggcaacactccttttttgccactatgcctacatggttcccggttggcaagctgaggcaatgcgctcagcccagcggcggacagcggccggaatcatgaagaacgctgtagtggatggcatcgtggccacggacgtcccagaattagagcgcaccacacccatcatgcagaagaaaattggacagatcatgctgatcttggtgtctctagctgcagtagtagtgaacccgtctgtgaagacagtacgagaagccggaattttgatcacggccgcagcggtgacgctttgggagaatggagcaagctctgtttggaacgcaacaactgccatcggactctgccacatcatgcgtgggggttggttgtcatgtctatccataacatggacactcataaagaacatggaaaaaccaggactaaaaagaggtggggcaaaaggacgcaccttgggagaggtttggaaagaaagactcaaccagatgacaaaagaagagttcactaggtaccgcaaagaggccatcatcgaagtcgatcgctcagcggcaaaacacgccaggaaagaaggcaatgtcactggagggcatccagtctctaggggcacagcaaaactgagatggctggtcgaacggaggtttctcgaaccggtcggaaaagtgattgaccttggatgtggaagaggcggttggtgttactatatggcaacccaaaaaagagtccaagaagtcagagggtacacaaagggcggtcccggacatgaagagccccaactagtgcaaagttatggatggaacattgtcaccatgaagagtggagtggatgtgttctacagaccttctgagtgttgtgacaccctcctttgtgacatcggagagtcctcgtcaagtgctgaggttgaagagcataggacgattcgggtccttgaaatggttgaggactggctgcaccgagggccaagggaattttgcgtgaaggtgctctgcccctacatgccgaaagtcatagagaagatggagctgctccaacgccggtatggggggggactggtcagaaacccactctcacggaattccacgcacgagatgtattgggtgagtcgagcttcaggcaatgtggtacattcagtgaatatgaccagccaggtgctcctaggaagaatggaaaaaaggacctggaagggaccccaatacgaggaagacgtaaacttgggaagtggaaccagggcggtgggaaaacccctgctcaactcagacaccagtaaaatcaagaacaggattgaacgactcaggcgtgagtacagttcgacgtggcaccacgatgagaaccacccatatagaacctggaactatcatggcagttatgatgtgaagcccacaggctccgccagttcgctggtcaatggagtggtcaggctcctctcaaaaccatgggacaccatcacgaatgttaccaccatggccatgactgacactactcccttcgggcagcagcgagtgttcaaagagaaggtggacacgaaagctcctgaaccgccagaaggagtgaagtacgtgctcaacgagaccaccaactggttgtgggcgtttttggccagagaaaaacgtcccagaatgtgctctcgagaggaattcataagaaaggtcaacagcaatgcagctttgggtgccatgtttgaagagcagaatcaatggaggagcgccagagaagcagttgaagatccaaaattttgggaaatggtggatgaggagcgcgaggcacatctgcggggggaatgtcacacttgcatttacaacatgatgggaaagagagagaaaaaacccggagagttcggaaaggccaagggaagcagagccatttggttcatgtggctcggagctcgctttctggagttcgaggctctgggttttctcaatgaagaccactggcttggaagaaagaactcaggaggaggtgtcgagggcttgggcctccaaaaactgggttacatcctgcgtgaagttggcatccggcctgggggcaagatctatgctgatgacacagctggctgggacacccgcatcacgagagctgacttggaaaatgaagctaaggtgcttgagctgcttgatggggaacatcggcgtcttgccagggccatcattgagctcacctatcgtcacaaagttgtgaaagtgatgcgcccggctgctgatggaagaaccgttatggatgttatctccagagaagatcagagggggagtggacaagttgtcacctacgccctaaacactttcaccaacctggctgtccagctggtgaggatgatggaaggggaaggagtgattggcccagatgatgtggagaaactcacaaaagggaaaggacccaaagtcaggacctggctgtttgagaatggggaagaaagactcagccgcatggctgtcagtggagatgactgtgtggtaaagcccctggacgatcgctttgccacctcgctccacttcctcaatgctatgtcaaaggttcgcaaagacatccaagagtggaaaccgtcaactggatggtatgattggcagcaggttccattttgctcaaaccatttcactgaattgatcatgaaagatggaagaacactggtggttccatgccgaggacaggatgaattggtaggcagagctcgcatatctccaggggccggatggaacgtccgcgacactgcttgtctggctaagtcttatgcccagatgtggctgcttctgtacttccacagaagagacctgcggctcatggccaacgccatttgctccgctgtccctgtgaattgggtccctaccggaagaaccacgtggtccatccatgcaggaggagagtggatgacaacagaggacatgttggaggtctggaaccgtgtttggatagaggagaatgaatggatggaagacaaaaccccagtggagaaatggagtgacgtcccatattcaggaaaacgagaggacatctggtgtggcagcctgattggcacaagagcccgagccacgtgggcagaaaacatccaggtggctatcaaccaagtcagagcaatcatcggagatgagaagtatgtggattacatgagttcactaaagagatatgaagacacaactttggttgaggacacagtactgtagatatttaatcaattgtaaatagacaatataagtatgcataaaagtgtagttttatagtagtatttagtggtgttagtgtaaatagttaagaaaatcttgaggagaaagtcaggccgggaagttcccgccaccggaagttgagtagacggtgctgcctgcgactcaaccccaggaggactgggtgaacaaagccgcgaagtgatccatgtaagccctcagaaccgtctcggaaggaggaccccacatgttgtaacttcaaagcccaatgtcagaccacgctacggcgtgctactctgcggagagtgcagtctgcgatagtgccccaggaggactgggttaacaaaggcaaaccaacgccccacgcggcccaagccccggtaatggtgttaaccagggcgaaaggactagaggttagaggagaccccgcggtttaaagtgcacggcccagcctggctgaagctgtaggtcaggggaaggactagaggttagtggagaccccgtgccacaaaacaccacaacaaaacagcaaatagacacctgggatagactaggagatcttctgctctgcacaaccagccacacggcacagtgcgccgacaatggtggctggtggtgcgagaacacaggatct

SEQ ID NO: 90 - DNA, 10495 bp, Artificial Sequence
  Synthetic Construct
tcatcaagcggacacactcgactgtttgaatcatcacaaacactcctaattgttgttaattgtgtcacgctcgacaaagaatcgtgcttctagagctacagattctttggtcctcccgggccgttctcggcccgacagataaacgattttgcgccttacggggcgcacaacaggaactaacctgaattcgttttcttcgctcccccgttctgaccatatcgacactagtaccttgacgggtagtagttccggttgcggtagtggtggtaggactagcggcactggaagacgaagcggtcgtcggtcttgtagtggctccttaagatggtctcgtggacgtcgcggcactcgttcccgatggactcgcgggacgcctggccgaccatgtggtcgcactagtggtagctcgacaggttgtagtttcttttgttcacgttgccgtggctgcggttccactttgactagttcgtccttgacctgttcatgttcttgcggcactggctcgacgtcgacgactacgtctcgtggggacggcggttgttggcccggtctgcgctcgacggggccaagtacttgatgtgggacttgttgcggttcttttggttgcactgggactcgttcttcgccttcgccgccaaggacccgaaggacgacccgcacccgtcgcggtagcggtcgccgtagcggcacaggttccacgacgtggaccttccgctccacttgttctagttcaggcgggacgacaggtggttgttccggcaccacagggactcgttgccgcactcgcacgactggtcgttccacgacctagacttcttgatgtagctgttcgtcgacgacgggtagcacttgttcgtctcgacgtcgtagtcgttgtagctctggcactagctcaaggtcgtcttcttgttggccgacgacctttagtgggccctcaagtcgcacttgcggccgcactggtgggggcactcgtggatgtacgactggttgtcgctcgacgacagggactagttactgtacgggtagtggttgctggtcttctttgactactcgttgttgcacgtctagcacgccgtcgtctcgatgaggtagtactcgtagtagtttcttctccacgaccggatgcaccacgtcgacggggacatgccgcactagctgtgggggacgaccttcgacgtgtggtcgggggacacgtggtggttgtggtttctcccgtcgttgtagacggactgggcctggctggccccgaccatgacgttgttgcggccgtcgcactcgaagaagggggaccggctgtggacgttccacgtctcgttggcccacaagacgctgtggtacttgtcggactgggacgggaggctccacttggacacgttgtagctgtagaagttggggttcatgctgacgttctagtactggaggttctggctgcactcgtcgaggcactagtggagggacccgcggtagcactcgacgatgccgttctggttcacgtggcggtcgttgttcttggccccgtagtagttctggaagtcgttgccgacgctgatgcactcgttgttcccgcacctgtggcactcgcacccgttgtgtgacatgatgcacttattcgtccttccgttctcggacatgcacttcccgctcggatagtagttgaagatgctgggggaccacaaggggtcgctgctcaagctgcggtcgtagtcggtccacttgctcttctagttggtctcggaccggaagtaggccttctcgctgctcgacgacgtgttacacttacggccgttctcgtggtggttatagtacttaaaactagacgagtttgaacgtccgctacatcttagtttaggacctgggcgggccctgtccaggtatcgagagtgcaaagagcgtcaacctcctcaagacgagaaggagaggcacttgcacgtgcgactgtgacccacacggtatctgtagtcggccgttctcgactctacaccttcacctcacaagtatgtgttactacacctccgaacctacctggccatgttcataatgggactttgcggtgttccggatcggttctagtaagtctttcgagtattccttcctcacacgccagatgctagtcaaaggtctgacctcgtagtttacacccttcgtcacttcctgctcgacttgtgagaaaacttcctcttaccacacctggaatcacagcaccaactctttgttcccccttacatgttcagtcgtggatttgcggagtggcggtggtgcctttttaacctttaaccgaccttccggacccctttctcataaaacaaacgtggtcttgagcggttgttgtggaaacaccaactaccaggcctctggttccttacaggctgagtcttagcgcgaaccttatcgaatcttcacctcctaaaacctaaaccagagtggtcgtgagcctacaaggacttccagtctctctcgttgtgttgacttacactgagcttctagtaaccttgccgacagttcttgttgaaccgctaggtgtcactggacaggataacctaactttcgtccgagttactatgcaccttcgaactttcccgtcaagacccacttcagtttagtacatgcaccggactctgcgtatggaacaccccgctaccttaggaactctcactgaactattatggtcagtgtgaccgccctggtgcttcgttagtgttagcctctggacccatattctgtgttttggtcccgggtaccctgcttccggcccatctctaactgaagctaatgacgggtccttgatgccagtgggactcactctcgacgcctgtggcacctggacggtgagcgtggtggtgtctctcgcctttcaactattgtctaaccacgacgtcctcgacgtggaatggtggtgacgcgatggtttgactgtcgccgacaaccataccatacctctagtctggtgtctctgtactacttttctgggagcacgtcagtgttcacttacgaatattacgactatactaactgggaaaagtcaacccggaagaccagcacaagaaccggtgggtcctccaggaagcgttctccacctgtcggttctagtcgtacggtcgatatgactaacgagacgatcaggaccacaaacccccgtaatgaatgtgactacacaatgcgatacagtagaaccacccccgtcgaaagcgtcttagattaagccctcctctgcaccatgtgaaccgcgagtaccgctggaagttctatgttggtcacaaataccaccgtagcaaagaatttcgctctacctggttggtcctcttgtaaaacaactacaaccgccgacaaaagaaagtttaccgaatagtgctacgggcggtttaagacgagaccctctagggactacacaacttaagtgaccgttatcgaacctactatgactctcggtattgtaagtgttgctgtagtttgcaccaacaaggcgacgatcgggacgattgtgggcccgactctacgaacttagacctacacatgtcctatgacgacaactaccagccttatccgtcgaactagtccctcttctcctcgcgtcgacgttttttctttcctcgttcagacgatacgaaccgagatcggagttgtcctgagaagttggggtactaggaacgacgacctgactaacgtacactagggttggcatttgcgcccaccgggcgttgacttcactactgtcgacagccggattacaaacggtagcagcctcccgaccgtctcgaactgtaactgaggtaccggtaaggttactgatagcgccccgagtacaaacgacgaaagcactaaagaccctttagttgtctatacacctaactctcttgccgcctgtaaaggaccctttcactacgtctctaatgtccgagctcgctttctcaactacacgccgaactactactacctttgaaggtcgagtacttactaggtcctcgtggaaccttctatacctacgagtcttaccagacagagcgctaatcacgcatgtgggggacccgttagaacgggagtcatcaacctaaaacctattgagaggttatgtgtttctctcctccgcacaacaccctgtgagggagtggtttcctcatgtttttccccctgtgctggtggccgcagatgtcctagtactgagcacccgacgagccgtcaatagttcgtcctcgtccgcactaccaacttccacaaaaggtgtgggaaaccgtatgttgttttcctcggcgaaactactcgcctctcccggcggacctgggtatgaccccgtcacagttcctcctagctgaaacaatgcctcctgggacctttaacgtcgtgttcaccttgcccgtcctactccacgtctactaacaccaccttggaccgttcttgcaattcttgcaggtctgctttggtccccacaagttttgtggacttcctctttagccccggcactgaaacctgaaggggtgaccttgtagtccgagtggttatcacctgtttttgccactacactaacccgaaataccgttacctcagtattacgggttgccgagtatgtattcgcgctatcacgtcccactttcctacctactcggttagggtcggcctaagcttggactctacgactccttttttgtctagtgacatgacctagaggtagggccgcggccattttgttcctcctaagacggtgtctagtagtttctccggtatttgtcttctgactcttgtcggcacgatcgtggttggtcccaacaccgacgactctaccgacttcgtgactctcctgacgggtaggccatggtctgtaggcgtcacgggtctcttgtattacctttactctaacaactacagtacacagtacgatgggagtgggtgtccgactacagaggagtgtcccacggcttgatgttggacaagcactacctactccgagtaaagtggctgggtcgatcgtaacgtcgttctccaatgtaaaggtgtttccagctcgatcccctccgccgccgttataagtactgtcggtggggtggtccgtgaagtctaggtaagggtctcaggttaagtggttaaaggctgaatgtctgactctagggcctagctcgaaccttgagacctatgcttacctagtgtcttatgtggcccttctgccaaaccaaacacggatcacaattctaccccttactctaacgggaaacggatgttgcacgacctttctttcatcaggttaacttgtctttcagcatgctctgcctcatgggttttacattcttgctactaaccctgaaacaatagtgttgtctgtatagactttacccccgattgaagttccgctcgtcccactaactgtcggccttctcacactttggttggtagtattgtcttcctcttccctctcactaggaccctcttggtagacgtcactgtcgtcgatcacggcgggtctctgcacctgcatagccatctttaggcagcgttcaaccactactcatgacaataccccccgtgtgcttacttctgctgagcttgaagcgggtaacctgactccgtgcttagtacgacctgttgtagttgtacggtttgcctgactagcgagttaagatggttggtctcgcactcttccatatatggtacctaccccttatggccgagtctcctcttctctcttttttgaaagaccttgacaactcctgacgtctagacggtcaaaccgaccgaatgttccaacgtcgccgacctcacagtatggtgctggcctccaccacgaaactaccaggatcctgtttgtgttaaaatcttctgttgttgcttcaccttcagtagtgcttcgaaccactttccttctaagactccggcgcgacctaactgcggtcccacatgagcctagtggtccgtgatttccgcaagttcctgaagcggagcccttttgcaagagtctatcccgagtaactccaagaccctttctacggactcgtgaagtaccccttctgtacccttcgtgaactgtggtacatgcaacaccggtgacgtctctttcctccttctcgagtgtcttaccgggacctccttgacggtctacgagaagtctgttaacggaactaacggaataactcacactactggtaccctcataagaaggaggagtacgtcgccttcccgtaacctttctatccaaaccctccgcgacagaaccctcagcgctggaaaaagacaacctaccgacttcaaggtccttgcttctagcggccttacaacgacgagagggaagagaactactaacacgattaaggactcggtctcttcgttgcaagcgtctgtctgttggtcgatcggcacaaggactatacacagtactgggaacactcgcgtcaccgtcggttgctctacccaaccgatctattctggttctcactgtattcgtcaaacaaacccgtttcttaactccagttcctcttaaagtcgtaccctctcaaagaagacctgaactccggccgttgtcggaccagtgacatgcgacactgttgtcgccaggagtgaggtgacgatttcgtaaactagtgcagtctaatgtagttgtggagtaactggagttatttgcaagtccgttcacgtgataagtgtgagcgcgctccgaaggggaagcagctacaacctcacagccgagaggacgatcgtcggcctacgacccctgttcagtgggagtggcaatgccattgtcgccgttgtgaggaaaaaacggtgatacggatgtaccaagggccaaccgttcgactccgttacgcgagtcgggtcgccgcctgtcgccggccttagtacttcttgcgacatcacctaccgtagcaccggtgcctgcagggtcttaatctcgcgtggtgtgggtagtacgtcttcttttaacctgtctagtacgactagaaccacagagatcgacgtcatcatcacttgggcagacacttctgtcatgctcttcggccttaaaactagtgccggcgtcgccactgcgaaaccctcttacctcgttcgagacaaaccttgcgttgttgacggtagcctgagacggtgtagtacgcacccccaaccaacagtacagataggtattgtacctgtgagtatttcttgtacctttttggtcctgatttttctccaccccgttttcctgcgtggaaccctctccaaacctttctttctgagttggtctactgttttcttctcaagtgatccatggcgtttctccggtagtagcttcagctagcgagtcgccgttttgtgcggtcctttcttccgttacagtgacctcccgtaggtcagagatccccgtgtcgttttgactctaccgaccagcttgcctccaaagagcttggccagccttttcactaactggaacctacaccttctccgccaaccacaatgatataccgttgggttttttctcaggttcttcagtctcccatgtgtttcccgccagggcctgtacttctcggggttgatcacgtttcaatacctaccttgtaacagtggtacttctcacctcacctacacaagatgtctggaagactcacaacactgtgggaggaaacactgtagcctctcaggagcagttcacgactccaacttctcgtatcctgctaagcccaggaactttaccaactcctgaccgacgtggctcccggttcccttaaaacgcacttccacgagacggggatgtacggctttcagtatctcttctacctcgacgaggttgcggccatacccccccctgaccagtctttgggtgagagtgccttaaggtgcgtgctctacataacccactcagctcgaagtccgttacaccatgtaagtcacttatactggtcggtccacgaggatccttcttaccttttttcctggaccttccctggggttatgctccttctgcatttgaacccttcaccttggtcccgccacccttttggggacgagttgagtctgtggtcattttagttcttgtcctaacttgctgagtccgcactcatgtcaagctgcaccgtggtgctactcttggtgggtatatcttggaccttgatagtaccgtcaatactacacttcgggtgtccgaggcggtcaagcgaccagttacctcaccagtccgaggagagttttggtaccctgtggtagtgcttacaatggtggtaccggtactgactgtgatgagggaagcccgtcgtcgctcacaagtttctcttccacctgtgctttcgaggacttggcggtcttcctcacttcatgcacgagttgctctggtggttgaccaacacccgcaaaaaccggtctctttttgcagggtcttacacgagagctctccttaagtattctttccagttgtcgttacgtcgaaacccacggtacaaacttctcgtcttagttacctcctcgcggtctcttcgtcaacttctaggttttaaaaccctttaccacctactcctcgcgctccgtgtagacgccccccttacagtgtgaacgtaaatgttgtactaccctttctctctcttttttgggcctctcaagcctttccggttcccttcgtctcggtaaaccaagtacaccgagcctcgagcgaaagacctcaagctccgagacccaaaagagttacttctggtgaccgaaccttctttcttgagtcctcctccacagctcccgaacccggaggtttttgacccaatgtaggacgcacttcaaccgtaggccggacccccgttctagatacgactactgtgtcgaccgaccctgtgggcgtagtgctctcgactgaaccttttacttcgattccacgaactcgacgaactaccccttgtagccgcagaacggtcccggtagtaactcgagtggatagcagtgtttcaacactttcactacgcgggccgacgactaccttcttggcaatacctacaatagaggtctcttctagtctccccctcacctgttcaacagtggatgcgggatttgtgaaagtggttggaccgacaggtcgaccactcctactaccttccccttcctcactaaccgggtctactacacctctttgagtgttttccctttcctgggtttcagtcctggaccgacaaactcttaccccttctttctgagtcggcgtaccgacagtcacctctactgacacaccatttcggggacctgctagcgaaacggtggagcgaggtgaaggagttacgatacagtttccaagcgtttctgtaggttctcacctttggcagttgacctaccatactaaccgtcgtccaaggtaaaacgagtttggtaaagtgacttaactagtactttctaccttcttgtgaccaccaaggtacggctcctgtcctacttaaccatccgtctcgagcgtatagaggtccccggcctaccttgcaggcgctgtgacgaacagaccgattcagaatacgggtctacaccgacgaagacatgaaggtgtcttctctggacgccgagtaccggttgcggtaaacgaggcgacagggacacttaacccagggatggccttcttggtgcaccaggtaggtacgtcctcctctcacctactgttgtctcctgtacaacctccagaccttggcacaaacctatctcctcttacttacctaccttctgttttggggtcacctctttacctcactgcagggtataagtccttttgctctcctgtagaccacaccgtcggactaaccgtgttctcgggctcggtgcacccgtcttttgtaggtccaccgatagttggttcagtctcgttagtagcctctactcttcatacacctaatgtactcaagtgatttctctatacttctgtgttgaaaccaactcctgtgtcatgacatctataaattagttaacatttatctgttatattcatacgtattttcacatcaaaatatcatcataaatcaccacaatcacatttatcaattcttttagaactcctctttcagtccggcccttcaagggcggtggccttcaactcatctgccacgacggacgctgagttggggtcctcctgacccacttgtttcggcgcttcactaggtacattcgggagtcttggcagagccttcctcctggggtgtacaacattgaagtttcgggttacagtctggtgcgatgccgcacgatgagacgcctctcacgtcagacgctatcacggggtcctcctgacccaattgtttccgtttggttgcggggtgcgccgggttcggggccattaccacaattggtcccgctttcctgatctccaatctcctctggggcgccaaatttcacgtgccgggtcggaccgacttcgacatccagtccccttcctgatctccaatcacctctggggcacggtgttttgtggtgttgttttgtcgtttatctgtggaccctatctgatcctctagaagacgagacgtgttggtcggtgtgccgtgtcacgcggctgttaccaccgaccaccacgctcttgtgtcctaga

SEQ ID NO: 91 - protein, 298 aa, Respiratory Syncytial Virus
MSKNKDQRTAKTLEKTWDTLNHLLFISSGLYKLNLKSVAQITLSILAMIISTSLIITAIIFIASANHKVTLTTAIIQDATSQIKNTTPTYLTQDPQLGISFSNLSEITSQTTTILASTTPGVKSNLQPTTVKTKNTTTTQTQPSKPTTKQRQNKPPNKPNNDFHFEVFNFVPCSICSNNPTCWAICKRIPNKKPGKKTTTKPTKKPTFKTTKKDLKPQTTKPKEVPTTKPTEEPTINTTKTNITTTLLTNNTTGNPKLTSQMGTFHSTSSEGNLSPSQVSTTSEHPSQPSSPPNTTRQ

SEQ ID NO: 92 - DNA, 920 bp, Respiratory Syncytial Virus
tgcaaacatgtccaaaaacaaggaccaacgcaccgctaagacactagaaaagacctgggacactctcaatcatttattattcatatcatcgggcttatataagttaaatcttaaatctgtagcacaaatcacattatccattctggcaatgataatctcaacttcacttataattacagccatcatattcatagcctcggcaaaccacaaagtcacactaacaactgcaatcatacaagatgcaacaagccagatcaagaacacaaccccaacatacctcactcaggatcctcagcttggaatcagcttctccaatctgtctgaaattacatcacaaaccaccaccatactagcttcaacaacaccaggagtcaagtcaaacctgcaacccacaacagtcaagactaaaaacacaacaacaacccaaacacaacccagcaagcccactacaaaacaacgccaaaacaaaccaccaaacaaacccaataatgattttcacttcgaagtgtttaactttgtaccctgcagcatatgcagcaacaatccaacctgctgggctatctgcaaaagaataccaaacaaaaaaccaggaaagaaaaccaccaccaagcctacaaaaaaaccaaccttcaagacaaccaaaaaagatctcaaacctcaaaccactaaaccaaaggaagtacccaccaccaagcccacagaagagccaaccatcaacaccaccaaaacaaacatcacaactacactgctcaccaacaacaccacaggaaatccaaaactcacaagtcaaatggaaaccttccactcaacctcctccgaaggcaatctaagcccttctcaagtctccacaacatccgagcacccatcacaaccctcatctccacccaacacaacacgccagtagttattaaaaaaaaaaa